(19) United States
(12) Patent Application Publication
(10) Pub. No.: US 2026/0356851 A1
(43) Pub. Date: Nov. 12, 2026

(54) TRICYCLIC COMPOUNDS AND USES THEREOF

(71) Applicant: HUTCHMED LIMITED, Shanghai (CN)

(72) Inventors: Weihan ZHANG, Shanghai (CN); Kun XIAO, Shanghai (CN); Zheng ZHANG, Shanghai (CN); Yao WU; Wei-Guo SU, Shanghai (CN)

(21) Appl. No.: 19/482,769

(22) PCT Filed: May 16, 2024
(86) PCT No.: PCT/CN2024/093647

§ 371 Date: Nov. 9, 2025

(30) Foreign Application Priority Data
May 16, 2023 (CN) 202310552365.4
Nov. 3, 2023 (CN) 202311459371.1
Feb. 1, 2024 (CN) 202410149082.X
May 9, 2024 (CN) 202410567016.4

(51) Int. Cl. C07D 498/16; A61K 31/519; A61K 31/553; A61P 35/00; C07D 519/00

(52) U.S. Cl. CPC C07D 498/16; A61K 31/519; A61K 31/553; A61P 35/00; C07D 519/00; C07B 2200/05

(57) ABSTRACT

The present invention relates to tricyclic compounds of formula (I), pharmaceutical compositions comprising same, preparation methods therefor and uses thereof, wherein each variable is as defined in the description.

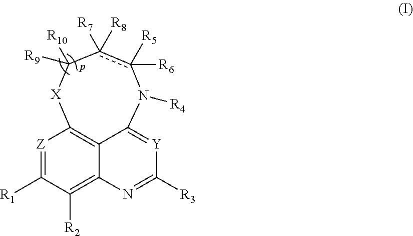

### TECHNICAL FIELD

The present invention relates to tricyclic compounds, pharmaceutical compositions comprising same, preparation methods therefor and uses thereof.

### BACKGROUND ART

RAS (rat sarcoma virus) proteins are a type of membrane-bound guanosine triphosphate hydrolase (a GTP hydrolase), and currently, there are three known genes in the RAS family: KRAS, NRAS and HRAS. The RAS protein functions as a molecular switch, which is activated and in an “ON” state when binding to GTP and is inactivated and in an “OFF” state when GTP is hydrolyzed. This process is regulated by a guanine nucleotide exchange factor (GEF) or a GTPase-activating protein (GAP): the GEF stimulates the exchange of guanosine diphosphate (GDP) with guanosine triphosphate (GTP), whereas the GAP promotes the hydrolysis of the GTP. Research has shown that the RAS genes have a high frequency of missense mutations in tumors, with three mutational hotspots at G12, G13, and Q61, respectively. These mutations can enable the binding of the RAS protein to GTP for a long time, resulting the continuous activation of the downstream effector signals, and driving the formation and development of tumors. KRAS is one of the important members of the RAS family, with the main downstream signaling pathways including PI3K/AKT, RAF/MEK/ERK, etc., which regulate various biological functions such as cell proliferation, apoptosis, and differentiation.

The abnormal activation of RAS proteins is closely related to tumorigenesis, among which KRAS mutations are the most common, accounting for about 85%, NRAS and HRAS accounting for 12% and 3%, respectively. The KRAS mutations are predominant in pancreatic cancer, colorectal cancer and lung cancer, the NRAS mutations are more common in melanoma and acute myeloid leukemia, and the HRAS mutations are more common in bladder cancer and head and neck cancer. The KRAS mutations are most common with G12 mutation at codon 12, G12C is the most common KRAS mutation in NSCLC patients, and KRAS G12D and KRAS G12V are the most common mutations in colorectal cancer and pancreatic cancer. Amplification of the KRAS gene has also been found in a variety of tumors, with an incidence of approximately 15% in esophageal adenocarcinoma and chromosomal instability gastric cancer.

Although the close relationship between KRAS and tumors has attracted much attention to this target, it took scientists more than thirty years of study to turn it into a targetable protein. In May 2021, the KRAS G12C inhibitor of Lumakras from the Amgen received accelerated approval from the US FDA, marking a significant breakthrough in breaking the “undruggability” of KRAS. However, the KRAS G12C patient population only accounts for a small portion of the KRAS mutation patients, and a large number of patients require medicaments targeting other KRAS mutations. Currently, no medicament against mutations other than the KRAS G12C mutation has been approved globally. Therefore, the development of KRAS targeted medicaments, in particular pan-KRAS targeted drugs, has broad application prospects.

### SUMMARY OF THE INVENTION

The present invention addresses the aforementioned needs in the field. The present invention provides KRAS inhibitors, which can significantly inhibit the KRAS mutations or KRAS gene amplification. In some examples, the compound of the present invention is a mutant KRAS G12C inhibitor. In some examples, the compound of the present invention is a mutant KRAS G12D inhibitor. In some examples, the compound of the present invention is a mutant KRAS G12V inhibitor. In some examples, the compound of the present invention is a mutant KRAS G13D inhibitor. In some examples, the compound of the present invention is a mutant KRAS Q61H inhibitor. In some examples, the compound of the present invention is a KRAS gene amplification inhibitor. In some examples, the compound of the present invention is a pan-KRAS inhibitor. The compounds of the present invention can be used for treating or preventing diseases containing KRAS mutations or KRAS gene amplification, especially cancer.

The present invention provides a compound of formula (I):

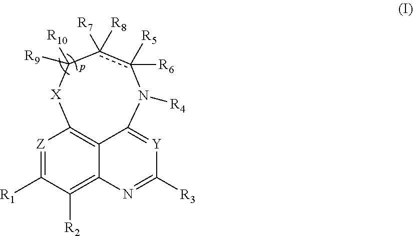

or a pharmaceutically acceptable salt thereof, or a solvate, a racemic mixture, an enantiomer, a diastereomer or a tautomer thereof, wherein
X is O, NR₁₁, CR₁₅R₁₋₆, C(O), S, S(O) or S(O)₂;
R₁₁ is independently selected from hydrogen, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, —CO(C₁₋₆ alkyl), —CONH₂, —CONH(C₁₋₆ alkyl), —CON(C₁₋₆ alkyl)₂, C₃₋₈ cycloalkyl, and 3-8 membered heterocyclyl;
R₁₅ and R₁₋₆ are each independently selected from hydrogen, deuterium, halogen, —CN, —OH, —NH₂, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O(C₁₋₆ alkyl), —O(C₁₋₆ haloalkyl), —NH(C₁₋₆ alkyl), —N(C₁₋₆ alkyl)₂, —CO(C₁₋₆ alkyl), —CONH₂, —CONH(C₁₋₆ alkyl), —CON(C₁₋₆ alkyl)₂, C₃₋₈ cycloalkyl, and 3-8 membered heterocyclyl; or R₁₅ and R₁₋₆ together with the carbon atom to which they are attached form C₃₋₆ cycloalkyl or 3-6 membered heterocyclyl;
Y is N, and Z is N; or Y is C—CN, and Z is N or Z is CR₂;
[symbol] represents a double bond or a single bond, and when [symbol] represents a double bond, R₆ and R₈ are absent;
p is 0, 1 or 2;
R₁ is —L₁—R₁₈;
L₁ is absent, or L₁ is NR₁₁, O, C(O), S, S(O) or S(O)₂;
R₁₈ is selected from 6-14 membered aryl, 5-14 membered heteroaryl, C₃₋₁₀ cycloalkyl, and 4-14 membered heterocyclyl, each of which is optionally substituted with one or more groups independently selected from: halogen, —CN, —OH, —SH, —SF₅, oxo, C₁₋₆ alkyl, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —O(C₃₋₈ cycloalkyl), —O(3-8 membered heterocyclyl), —S(C₁₋₆ alkyl), —S(C₁₋₆ haloalkyl), —S(C₃₋₈ cycloalkyl), —S(3-8 membered heterocyclyl), —OC(O)NRₐR_{b}, —NRₐC(O)OR_{c}, —NRₐC(O)Rₐ, —NRₐR_{b}, —C(O)NRₐR_{b}, —C(O)OR_{c}, —C(O)R_{d}, —S(O)₂R_{f}, and —S(O)₂NRₐR_{b}; wherein the C₁₋₆ alkyl is optionally substituted with one or more deuterium, and the C₂₋₆ alkenyl, C₂₋₆ alkynyl, C₃₋₈ cycloalkyl and 3-8 membered heterocyclyl are each optionally substituted with one or more halogen;
R₂ is selected from hydrogen, halogen, —CN, —OH, —SH, oxo, C₁₋₆ alkyl, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —O(C₃₋₈ cycloalkyl), —O(3-8 membered heterocyclyl), —S(C₁₋₆ alkyl), —S(C₁₋₆ haloalkyl), —S(C₃₋₈ cycloalkyl), —S(3-8 membered heterocyclyl), —OC(O)NRₐR_{b}, —NRₐC(O)OR_{c}, —NRₐC(O)R_{d}, —NRₐR_{b}, —C(O)NRₐR_{b}, —C(O)OR_{c}, —C(O)R_{d}, —S(O)₂R_{f}, and —S(O)₂NRₐR_{b}; wherein the C₁₋₆ alkyl is optionally substituted with one or more deuterium, and the C₂₋₆ alkenyl, C₂₋₆ alkynyl, C₃₋₈ cycloalkyl and 3-8 membered heterocyclyl are each optionally substituted with one or more halogen;
R₃ is selected from hydrogen, —OH, —SH, —NH₂, C₁₋₆ alkyl, C₁₋₆ haloalkyl, —O(C₁₋₆ alkyl), —O(C₁₋₆ haloalkyl), —S(C₁₋₆ alkyl), —S(C₁₋₆ haloalkyl), —NH(C₁₋₆ alkyl), —N(C₁₋₆ alkyl)₂, C₃₋₈ cycloalkyl, 4-10 membered heterocyclyl, and —O—L₂-R₁₂; wherein the C₃₋₈ cycloalkyl and 4-10 membered heterocyclyl are each optionally substituted with one or more groups independently selected from: deuterium, halogen, —CN, —OH, —SH, oxo, —NH₂, C₁₋₆ alkyl, C₁₋₆ alkylidene, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)—CN, —O(C₁₋₆ alkyl), and —S(C₁₋₆ alkyl), wherein the C₁₋₆ alkylidene is optionally substituted with one or more halogen;
L₂ is absent, or L₂ is C₁₋₆ alkylene or C₃₋₈ cycloalkylene, wherein the C₁₋₆ alkylene and C₃₋₈ cycloalkylene are each optionally substituted with one or more deuterium or halogen;
R₁₂ is C₃₋₁₀ cycloalkyl or 4-12 membered heterocyclyl, each of which is optionally substituted with one or more groups independently selected from: deuterium, halogen, —CN, —OH, —SH, oxo, C₁₋₆ alkyl, C₁₋₆ alkylidene, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-O—(C₁₋₆ alkyl), —O(C₁₋₆ alkyl), —O(C₁₋₆ haloalkyl), —O(C₃₋₈ cycloalkyl), —O(3-8 membered heterocyclyl), —S(C₁₋₆ alkyl), —S(C₁₋₆ haloalkyl), —S(C₃₋₈ cycloalkyl), —S(3-8 membered heterocyclyl), —OC(O)NRₐR_{b}, —NRₐC(O)OR_{c}, —NRₐC(O)R_{d}, —NRₐR_{b}, —C(O)NRₐR_{b}, —C(O)OR_{d}, —C(O)R_{d}, —S(O)₂R_{f}, and —S(O)₂NRₐR_{b}; wherein the C₁₋₆ alkyl, C₁₋₆, alkylidene, C₂₋₆ alkenyl, C₂₋₆ alkynyl, C₃₋₈ cycloalkyl and 3-8 membered heterocyclyl are each optionally substituted with one or more R₁₇;
R₁₇ is selected from deuterium, halogen, —CN, —OH, —SH, oxo, C₁₋₆ alkyl, C₁₋₆ alkylidene, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —S(C₁₋₆ alkyl), —S(C₁₋₆ haloalkyl), —NRₐR_{b}, —NHCO(C₁₋₆ alkyl), —CO(C₁₋₆ alkyl), —CONH₂, —CONH(C₁₋₆ alkyl), —CON(C₁₋₆ alkyl)₂, —S(O)₂(C₁₋₆ alkyl), —S(O)₂NH(C₁₋₆ alkyl), and —S(O)₂N(C₁₋₆ alkyl)₂; wherein the 3-8 membered heterocyclyl is optionally substituted with one or more groups independently selected from: deuterium, halogen, —CN, —OH, oxo, —NH₂, C₁₋₆ alkyl, C₁₋₆ alkylidene, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —S(C₁₋₆ alkyl), —S(C₁₋₆ haloalkyl), —CO(C₁₋₆ alkyl), and —S(O)₂(C₁₋₆ alkyl);
R₄ is selected from C₁₋₆ alkyl, C₂₋₆ alkenyl, C₃₋₁₀ cycloalkyl, 4-12 membered heterocyclyl and 5-12 membered heteroaryl, each of which is optionally substituted with one or more R₁₃;
R₁₃ is selected from deuterium, halogen, —CN, —OH, —SH, oxo, —NH₂, C₁₋₆ alkyl, C₂₋₆ alkenyl, C₂₋₆ alkynyl, C₁₋₆ haloalkyl, C₃₋₈ cycloalkyl, 3-8 membered heterocyclyl, 6-10 membered aryl, 5-10 membered heteroaryl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —S(C₁₋₆ alkyl), —S(C₁₋₆ haloalkyl), —NHCO(C₁₋₆ alkyl), —NH(C₁₋₆ alkyl), —N(C₁₋₆ alkyl)₂, —CO(C₁₋₆ alkyl), —CONH₂, —CONH(C₁₋₆ alkyl), —CON(C₁₋₆ alkyl)₂, —S(O)₂(C₁₋₆ alkyl), —S(O)₂NH(C₁₋₆ alkyl), and —S(O)₂N(C₁₋₆ alkyl)₂; wherein the C₃₋₈ cycloalkyl, 3-8 membered heterocyclyl, 6-10 membered aryl and 5-10 membered heteroaryl are each optionally substituted with one or more R₁₄;
R₁₄ is independently selected from deuterium, halogen, —CN, —OH, —SH, oxo, C₁₋₆ alkyl, C₁₋₆ haloalkyl, C₃₋₈ cycloalkyl, 3-8 membered heterocyclyl, phenyl, 5-6 membered heteroaryl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —O(C₃₋₈ cycloalkyl), —O(3-8 membered heterocyclyl), —S(C₁₋₆ alkyl), —S(C₁₋₆ haloalkyl), —S(C₃₋₈ cycloalkyl), —S(3-8 membered heterocyclyl), —NRₐC(O)R_{d}, —NRₐR_{b}, —C(O)NRₐR_{b}, —C(O)OR_{c}, —C(O)R_{d}, —S(O)₂R_{f}, and —S(O)₂NRₐR_{b}; wherein the C₁₋₆ alkyl is optionally substituted with one or more deuterium, and the C₃₋₈ cycloalkyl, 3-8 membered heterocyclyl, phenyl and 5-6 membered heteroaryl are each optionally substituted with one or more halogen;
R₅, R₆, R₇, R₈, R₉ and R₁₀ are each independently selected from hydrogen, deuterium, halogen, —CN, —OH, —SH, oxo, —NH₂, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O(C₁₋₆ alkyl), —O(C₁₋₆ haloalkyl), —NH(C₁₋₆ alkyl), —N(C₁₋₆ alkyl)₂, —CO(C₁₋₆ alkyl), —CONH₂, —CONH(C₁₋₆ alkyl), —CON(C₁₋₆ alkyl)₂, C₃₋₈ cycloalkyl, and 3-8 membered heterocyclyl; or R₅ and R₆, R₇ and R₈, R₉ and R₁₀, R₅ and R₇, R₇ and R₉, or R₅ and R₉ together with the carbon atoms to which they are attached form C₃₋₆ cycloalkyl or 3-6 membered heterocyclyl;
or R₄ and R₅ together with the nitrogen atom and carbon atom to which they are attached form 5-12 membered heteroaryl, which is optionally substituted with one or more R₁4;
or R₄ and R₅ together with the nitrogen atom and carbon atom to which they are attached form 4-10 membered heterocyclyl; wherein the 4-10 membered heterocyclyl is substituted with one or more groups independently selected from: C₃₋₈ cycloalkyl, 3-8 membered heterocyclyl, 6-10 membered aryl, and 5-10 membered heteroaryl; and the 4-10 membered heterocyclyl is further optionally substituted with one or more groups independently selected from: deuterium, halogen, —CN, —OH, —SH, oxo, —NH₂, C₁₋₆ alkyl, C₂₋₆ alkenyl, C₂₋₆ alkynyl, C₁₋₆ haloalkyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —S(C₁₋₆ alkyl), —S(C₁₋₆ haloalkyl), —NHCO(C₁₋₆ alkyl), —NH(C₁₋₆ alkyl), —N(C₁₋₆ alkyl)₂, —CO(C₁₋₆ alkyl), —CONH₂, —CONH(C₁₋₆ alkyl), —CON(C₁₋₆ alkyl)₂, —S(O)₂(C₁₋₆ alkyl), —S(O)₂NH(C₁₋₆ alkyl), and —S(O)₂N(C₁₋₆alkyl)₂; wherein the C₃₋₈ cycloalkyl, 3-8 membered heterocyclyl, 6-10 membered aryl and 5-10 membered heteroaryl are each optionally substituted with one or more R₁₄; and
Rₐ, R_{b}, R_{c}, R_{d} and R_{f} are each independently selected from hydrogen, C₁₋₆ alkyl, C₃₋₈ cycloalkyl, 3-8 membered heterocyclyl, phenyl, and 5-6 membered heteroaryl; wherein the C₁₋₆ alkyl, C₃₋₈ cycloalkyl, 3-8 membered heterocyclyl, phenyl and 5-6 membered heteroaryl are each optionally substituted with one or more groups independently selected from: halogen, —CN, —OH, —O(C₁₋₆ alkyl), C₃₋₈ cycloalkyl, 3-8 membered heterocyclyl, —NR_{g}Rₕ, —C(O)NR_{g}Rₕ, and —NR_{g}C(O)Rᵢ; wherein R_{g}, Rₕ, and Rᵢ are each independently selected from hydrogen, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, —(C₁₋₆ alkyl)-NH₂, —(C₁₋₆ alkyl)-NH(C₁₋₆ alkyl), —(C₁₋₆ alkyl)-N(C₁₋₆ alkyl)₂, C₃₋₈ cycloalkyl, 3-8 membered heterocyclyl, phenyl, and 5-6 membered heteroaryl; or Rₐ and R_{b} together with the nitrogen atom to which they are attached form 3-8 membered heterocyclyl.

The above-mentioned compounds and the active compounds (including compounds of general formulas and specific compounds) disclosed in the context of the present invention, and pharmaceutically acceptable salts thereof, or solvates, racemic mixtures, enantiomers, diastereomers or tautomers thereof are collectively referred to herein as “compounds of the present invention”.

The present invention also provides a pharmaceutical composition, comprising the compounds of the present invention, and optionally comprising a pharmaceutically acceptable excipient.

The present invention also provides a method of in vivo or in vitro inhibiting the activity of mutant KRAS proteins, comprising contacting the mutant KRAS proteins with an effective amount of the compounds of the present invention.

The present invention also provides a method of treating or preventing a disease containing KRAS mutations or KRAS gene amplification, especially cancer, comprising administering to a subject in need thereof an effective amount of the compounds of the present invention.

The present invention also provides a method of treating or preventing cancer, especially cancer containing KRAS mutations or KRAS gene amplification, comprising administering to a subject in need thereof an effective amount of the compounds of the present invention.

The present invention also provides the use of the compounds of the present invention in the treatment or prevention of a disease containing KRAS mutations or KRAS gene amplification, especially cancer.

The present invention also provides the use of the compounds of the present invention in the treatment or prevention of cancer, especially cancer containing KRAS mutations or KRAS gene amplification.

The present invention also provides the use of the compounds of the present invention in the manufacture of a medicament for treating or preventing a disease containing KRAS mutations or KRAS gene amplification, especially cancer.

The present invention also provides the use of the compounds of the present invention in the manufacture of a medicament for treating or preventing cancer, especially cancer containing KRAS mutations or KRAS gene amplification.

The present invention also provides the compounds of the present invention for in vivo or in vitro inhibiting the activity of mutant KRAS proteins.

The present invention also provides the compounds of the present invention for use as a medicament.

The present invention also provides the compounds of the present invention for use as a medicament for treating or preventing a disease containing KRAS mutations or KRAS gene amplification, especially for treating or preventing cancer.

The present invention also provides a pharmaceutical combination, comprising the compounds of the present invention and at least one additional therapeutic agent, wherein the additional therapeutic agent is preferably selected from: an anti-neoplastic active agent, an anti-inflammatory agent or an immunomodulator, wherein the anti-neoplastic active agent includes a chemotherapeutic agent, an immune checkpoint inhibitor or agonist, and a targeted therapeutic agent.

The present invention also provides a kit for treating or preventing a disease containing KRAS mutations or KRAS gene amplification, especially cancer. The kit can comprise the pharmaceutical composition of the present invention and instructions for use, and the pharmaceutical composition comprises the compounds of the present invention.

### DETAILED DESCRIPTION OF THE INVENTION

### Definitions

As used in the present application, the following words, phrases and symbols are generally intended to have the meanings as set forth below, except to the extent that the context in which they are used indicates otherwise.

A dash (“-”) that is not between two letters or symbols is used to indicate a point of attachment for a substituent. For example, —O(C₁₋₆ alkyl) refers to the attachment of C₁₋₆ alkyl to the rest of the molecule through an oxygen atom.

The term “alkyl” as used herein refers to a straight or branched saturated hydrocarbon radical containing 1-18 carbon atoms (C₁₋₁₈), preferably 1-10 carbon atoms (C₁₋₁₀), more preferably 1-6 carbon atoms (C₁₋₆), and further more preferably 1-4 carbon atoms (C₁₋₄) or 1-3 carbon atoms (C₁₋₃). When the term “alkyl” is prefixed with “C_{a-b}”, it means the number of carbon atoms in the alkyl, where a is the minimum number of carbons in the alkyl and b is the maximum number of carbons in the alkyl. For example, “C₁₋₆ alkyl” refers to an alkyl containing 1-6 carbon atoms. “C₁₋₃ alkyl” refers to an alkyl containing 1-3 carbon atoms. Examples of C₁₋₆ alkyl include, but are not limited to, methyl, ethyl, propyl (e.g. n-propyl, i-propyl), butyl (e.g. n-butyl, i-butyl, s-butyl and t-butyl), pentyl (e.g. n-pentyl, i-pentyl, neo-pentyl), hexyl, and the like. When used between two dashes (“-”) (e.g., —(C₁₋₆ alkyl)-OH), the alkyl refers to an alkylene.

The term “alkylidene” as used herein refers to divalent radical of alkyl as defined above that is attached by two single bonds on the same carbon atom together to the rest of the molecule, i.e. straight or branched divalent hydrocarbon radical having formula ═CR′R″, where R′ and R″ can be the same or different. In one embodiment, an alkylidene radical is 1 to 6 carbon atoms (C₁₋₆ alkylidene). In another embodiment, the alkylidene radical is C₁₋₃ alkylidene, C₁₋₂ alkylidene, or C₁ alkylidene. Examples of C₁₋₆ alkylidene include, but are not limited to, methylidene (═CH₂), ethylidene (═CHCH₃), and propylidene (═CH—CH₂—CH₃).

The term “alkylene” as used herein refers to divalent radical of alkyl as defined above that is attached by two single bonds on the same or different carbon atoms respectively to the rest of the molecule. In one embodiment, an alkylene radical is 1 to 6 carbon atoms (C₁₋₆ alkylene). In another embodiment, the alkylene radical is C₁₋₃ alkylene, C₁₋₂ alkylene, or C₁ alkylene. Examples of C₁₋₆ alkylene include, but are not limited to, methylene (—CH₂—), 1,2-ethylene (—CH₂CH₂—), 1,1-ethylene (—CH(CH₃)—), 1,3-propylene (—CH₂—CH₂—CH₂—), 1,1-propylene (—CH(CH₂CH₃)—), 2,2-propylene (—C(CH₃)₂—), 1,2-propylene (—CH(CH₃)CH₂—), 1,4-butylene (—CH₂—CH₂—CH₂—CH₂—), and the like.

The term “alkenyl” as used herein refers to a straight or branched unsaturated hydrocarbon radical containing one or more, for example 1, 2, or 3 carbon-carbon double bonds (C═C) and 2-18 carbon atoms (C₂-18), preferably 2-10 carbon atoms (C₂₋₁₀), more preferably 2-6 carbon atoms (C₂₋₆), and further more preferably 2-4 carbon atoms (C₂₋₄). When the term “alkenyl” is prefixed with “C_{a-b}”, it means the number of carbon atoms in the alkenyl, where a is the minimum number of carbons in the alkenyl and b is the maximum number of carbons in the alkenyl. For example, “C₂₋₆ alkenyl” refers to an alkenyl containing 2-6 carbon atoms. “C₂₋₄ alkenyl” refers to an alkenyl containing 2-4 carbon atoms. Examples of C₂₋₆ alkenyl include, but are not limited to, vinyl, propenyl (e.g. 2-propenyl), and butenyl (e.g. 2-butenyl), and the like. The point of attachment for the alkenyl can be on or not on the double bonds.

The term “alkynyl” as used herein refers to a straight or branched unsaturated hydrocarbon radical containing one or more, for example 1, 2, or 3, carbon-carbon triple bonds (C≡C) and 2-18 carbon atoms (C₂₋₁₈), preferably 2-10 carbon atoms (C₂₋₁₀), more preferably 2-6 carbon atoms (C₂₋₆), and further more preferably 2-4 carbon atoms (C₂₋₄). When the term “alkynyl” is prefixed with “C_{a-b}”, it means the number of carbon atoms in the alkynyl, where a is the minimum number of carbons in the alkynyl and b is the maximum number of carbons in the alkynyl. For example, “C₂₋₆ alkynyl” refers to an alkynyl containing 2-6 carbon atoms. “C₂₋₄ alkynyl” refers to an alkynyl containing 2-4 carbon atoms. Examples of C₂₋₆ alkynyl include, but are not limited to, ethynyl, propynyl (e.g. 2-propynyl), and butynyl (e.g. 2-butynyl), and the like. The point of attachment for the alkynyl can be on or not on the triple bonds.

The term “halogen” or “halo” as used herein means fluoro, chloro, bromo, and iodo, preferably fluoro, chloro and bromo, more preferably fluoro and chloro.

The term “haloalkyl” as used herein refers to an alkyl radical, as defined herein, in which one or more, for example 1, 2, 3, 4, or 5, or all hydrogen atoms are replaced with halogen atoms, and when more than one hydrogen atoms are replaced with halogen atoms, the halogen atoms may be the same or different from each other. In one embodiment, the term “haloalkyl” as used herein refers to an alkyl radical, as defined herein, in which two or more, such as 2, 3, 4, or 5, or all hydrogen atoms are replaced with halogen atoms, wherein the halogen atoms are identical to each other. In another embodiment, the term “haloalkyl” as used herein refers to an alkyl radical, as defined herein, in which two or more hydrogen atoms, such as 2, 3, 4, or 5, or all hydrogen atoms are replaced with halogen atoms, wherein the halogen atoms are different from each other. When the term “haloalkyl” is prefixed with “C_{a-b}”, it means the number of carbon atoms in the haloalkyl, where a is the minimum number of carbons in the haloalkyl and b is the maximum number of carbons in the haloalkyl. For example, “C₁₋₆ haloalkyl” refers to a haloalkyl as defined herein containing 1-6 carbon atoms. “C₁₋₄ haloalkyl” refers to a haloalkyl as defined herein containing 1-4 carbon atoms. Examples of C₁₋₆ haloalkyl include, but are not limited to —CF₃, —CHF₂, —CH₂F, —CH₂CH₂F, —CH₂CHF₂, —CH₂CF₃, —CH(CF₃)₂, and the like.

The term “cycloalkyl” as used herein refers to saturated or partially unsaturated cyclic hydrocarbon radical having 3-12 ring carbon atoms (C₃₋₁₂), such as 3-10 ring carbon atoms (C₃₋₁₀), 3-8 ring carbon atoms (C₃₋₈), 5-7 ring carbon atoms (C₅₋₇), 4-7 ring carbon atoms (C₄₋₇) or 3-6 ring carbon atoms (C₃₋₆), which may have one or more rings, such as 1, 2, or 3 rings, preferably 1 or 2 rings. When the term “cycloalkyl” is prefixed with “C_{a-b}”, it means the number of carbon atoms in the cycloalkyl, where a is the minimum number of carbons in the cycloalkyl and b is the maximum number of carbons in the cycloalkyl. For example, “C₃₋₈ cycloalkyl” or “3-8 membered cycloalkyl” refers to a cycloalkyl containing 3-8 ring carbon atoms; “C₃₋₆ cycloalkyl” or “3-6 membered cycloalkyl” refers to a cycloalkyl containing 3-6 ring carbon atoms. The cycloalkyl may include a fused or bridged ring, or a spirocyclic ring. The rings of the cycloalkyl may be saturated or have one or more, for example, one or two double bonds (i.e. partially unsaturated), but not fully conjugated, and not an aryl as defined herein. Examples of cycloalkyl include, but are not limited to cyclopropyl, cyclobutyl, cyclopentyl, cyclohexyl, spiro[2.2]pentyl, cyclopropenyl, cyclobutenyl, cyclopentenyl, cyclopentadienyl, cyclohexenyl, etc.

The term “cycloalkylene” as used herein refers to divalent radical of cycloalkyl as defined above that is attached by two single bonds on the same or different carbon atoms respectively to the rest of the molecule. Examples of cycloalkylene include, but are not limited to, cyclopropylene (such as 1,1-cyclopropylene, 1,2-cyclopropylene), cyclobutylene (such as 1,1-cyclobutylene, 1,3-cyclobutylene), cyclopentylene, cyclohexylene, cyclopentenylene, cyclopentadienylene, cyclohexenylene, and the like.

The term “heterocyclyl” or “heterocycle” as used herein can be used interchangeably and each refers to saturated or partially unsaturated cyclic radicals having 3-14 ring atoms, such as 4-14 ring atoms (4-14 membered heterocyclyl), 4-12 ring atoms (4-12 membered heterocyclyl), 4-10 ring atoms (4-10 membered heterocyclyl), 3-8 ring atoms (3-8 membered heterocyclyl), 4-8 ring atoms (4-8 membered heterocyclyl), 3-6 ring atoms (3-6 membered heterocyclyl) or 4-5 ring atoms (4-5 membered heterocyclyl), and containing one or more, for example 1, 2 or 3, preferably 1 or 2 heteroatoms independently selected from N, O and S in the rings, with the remaining ring atoms being carbon; it may have one or more rings, for example 1, 2 or 3, preferably 1 or 2 rings. The heterocyclyl also includes those wherein the N or S heteroatom are optionally oxidized to various oxidation states. The point of attachment of heterocyclyl can be on the N heteroatom or carbon. For example, “4-10 membered heterocyclyl” represents a heterocyclyl having 4-10 (4, 5, 6, 7, 8, 9 or 10) ring atoms comprising at least one, such as 1, 2, 3 or 4, preferably 1, 2 or 3 heteroatoms independently selected from N, O and S; “3-8 membered heterocyclyl” represents a heterocyclyl having 3-8 (3, 4, 5, 6, 7 or 8) ring atoms comprising at least one, such as 1, 2 or 3, preferably 1 or 2 heteroatoms independently selected from N, O and S; and “3-6 membered heterocyclyl” represents a heterocyclyl having 3-6 (3, 4, 5 or 6) ring atoms comprising at least one, preferably 1 or 2 heteroatoms independently selected from N, O and S (preferably N and O), which is preferably a monocyclic ring. The heterocyclyl also includes a fused or bridged ring, or a spirocyclic ring. The rings of the heterocyclyl may be saturated or have one or more, for example, one or two double bonds (i.e. partially unsaturated), but not fully conjugated, and not a heteroaryl as defined herein. Examples of heterocyclyl include, but are not limited to: 4-10 membered heterocyclyl, 3-8 membered heterocyclyl, 3-6 membered heterocyclyl and 4-5 membered heterocyclyl, such as oxetanyl, azetidinyl, pyrrolidinyl, tetrahydrofuranyl, dioxolanyl, tetrahydropyranyl, morpholinyl, thiomorpholinyl, piperidinyl, piperazinyl, tetrahydropyridyl, dihydropyrimidinyl, dihydropyrazinyl, pyrazolidinyl, hexahydro-1H-pyrrolizinyl, hexahydrospiro[cyclopropane-pyrrolizinyl](such as tetrahydro-1′H,3′H-spiro[cyclopropane-1,2′-pyrrolizinyl], hexahydrospiro[cyclopropane-1,3′-pyrrolizinyl]and hexahydrospiro[cyclopropane-1,1′-pyrrolizinyl]), octahydrocyclopropa[a]pyrrolizinyl, and oxaspiro[3.3]heptyl, preferably oxetanyl, azetidinyl, pyrrolidinyl, tetrahydropyranyl, morpholinyl, piperidinyl, piperazinyl, hexahydro-1H-pyrrolizinyl, tetrahydro-1′H,3′H-spiro[cyclopropane-1,2′-pyrrolizinyl], octahydrocyclopropa[a]pyrrolizinyl.

The term “aryl” or “aromatic hydrocarbon” as used herein can be used interchangeably and each refers to carbocyclic hydrocarbon radical of 6 to 14 carbon atoms (such as 6-14 carbon atoms (6-14 membered aryl), 6-12 carbon atoms (6-12 membered aryl), 6-10 carbon atoms (6-10 membered aryl)), consisting of one ring or more, such as two fused rings, wherein at least one ring is an aromatic ring. Examples of aryl include, but are not limited to phenyl, naphthyl, 1,2,3,4-tetrahydronaphthyl, phenanthryl, indenyl, indanyl, azulenyl, benzocyclobutenyl, preferably phenyl and naphthyl.

The term “heteroaryl” or “heteroaromatic ring” as used herein can be used interchangeably and each refers to: mono-, bi-, or tri-ring system having 5-15 ring atoms (such as 5-14 ring atoms (5-14 membered heteroaryl), 5-13 ring atoms (5-13 membered heteroaryl), 5-12 ring atoms (5-12 membered heteroaryl), 5-6 ring atoms (5-6 membered heteroaryl), 8-13 ring atoms (8-13 membered heteroaryl), 9-12 ring atoms (9-12 membered heteroaryl)), and containing one or more, for example 1, 2, 3 or 4, preferably 1, 2 or 3 heteroatoms independently selected from N, O and S in the rings, with the remaining ring atoms being carbon, wherein at least one ring is an aromatic ring, wherein S and N may be optionally oxidized to various oxidation states. When the total number of S and O atoms in the heteroaryl group exceeds 1, said S and O heteroatoms are not adjacent to one another. Preferably, the heteroaryl is 5-13 membered heteroaryl. For example, the heteroaryl includes:
a 5-6 membered monocyclic heteroaryl, i.e., a monocyclic ring aromatic heterocyclic radical having 5 or 6 ring atoms, wherein the ring atoms include one or more, such as 1, 2 or 3 heteroatoms independently selected from N, O and S (preferably N), and the remaining ring atoms are carbon atoms; and
a 8-13 membered bicyclic or tricyclic heteroaryl, i.e., a bicycle or tricyclic aromatic heterocyclic radical having 8, 9, 10, 11, 12 or 13 ring atoms, wherein the ring atoms include one or more, such as 1, 2, 3 or 4, preferably 1, 2 or 3 heteroatoms independently selected from N, O and S (preferably N), and the remaining ring atoms are carbon atoms, wherein at least one ring is an aromatic ring.

Examples of heteroaryl include, but are not limited to, 5-6 membered monocyclic heteroaryl, such as pyridyl, N-oxide pyridyl, pyrazinyl, pyrimidinyl, triazinyl (such as 1,2,4-triazinyl, 1,3,5-triazinyl), pyrazolyl, imidazolyl, oxazolyl, isoxazolyl, oxadiazolyl (such as 1,2,4-oxadiazolyl, 1,2,5-oxadiazolyl, and 1,3,4-oxadiazolyl,), thiazolyl, isothiazolyl, thiadiazolyl, tetrazolyl, triazolyl, thienyl, furanyl, pyranyl, pyrrolyl, pyridazinyl, preferably triazolyl, pyridyl, N-oxide pyridyl, pyrazinyl, pyridazinyl, pyrimidinyl, pyrazolyl, imidazolyl, isoxazolyl, triazinyl (such as 1,2,4-triazinyl), oxazolyl, thiadiazolyl, more preferably pyridyl (such as pyridin-4-yl, pyridin-3-yl), pyrazinyl, pyridazinyl, pyrimidinyl; and 8-13 membered bicyclic or tricyclic heteroaryl, such as benzoxazolyl, benzoisoxazolyl, benzothienyl, benzothiazolyl, benzoisothiazolyl, imidazopyrimidinyl (such as imidazo[1,2-c]pyrimidinyl), imidazopyrazinyl (such as imidazo[1,2-a]pyrazinyl and imidazo[1,5-a]pyrazinyl), imidazopyridyl (such as imidazo[1,2-a]pyridyl), imidazopyridazinyl (such as imidazo[1,2-b]pyridazinyl), pyrrolopyrazinyl (such as pyrrolo[1,2-a]pyrazinyl), pyrrolopyridyl (such as 1H-pyrrolo[2,3-b]pyridyl), pyrrolopyrimidinyl (such as pyrrolo[3,4-d]pyrimidinyl), pyrazolopyrazinyl (such as pyrazolo[1,5-a]pyrazinyl), pyrazolopyridyl (such as 1H-pyrazolo[3,4-b]pyridyl), pyrazolopyrimidinyl (such as pyrazolo[1,5-a]pyrimidinyl), triazolopyrimidinyl (such as [1,2,4]triazolo[4,3-c]pyrimidinyl and [1,2,4]triazolo[1,5-c]pyrimidinyl), triazolopyrazinyl (such as [1,2,4]triazolo[1,5-a]pyrazinyl), triazolopyridyl (such as [1,2,4]triazolo[4,3-a]pyridyl and [1,2,4]triazolo[1,5-a]pyridyl), tetrazolopyridyl (such as tetrazolo[1,5-a]pyridyl), benzofuranyl, benzimidazolyl, indolyl, indazolyl, purinyl, quinolyl, quinolinonyl (i.e., oxoquinolyl, such as 2-oxoquinolyl), tetrahydroquinolyl (such as 1,2,3,4-tetrahydroquinolyl), isoquinolinyl, tetrahydroisoquinolyl (such as 5,6,7,8-tetrahydroisoquinolyl), tetrahydronaphthyridinyl (such as 1,2,3,4-tetrahydro-2,7-naphthyridinyl), dihydro-cyclopentapyridyl (such as 6,7-dihydro-5H-cyclopenta[c]pyridyl), dihydro-pyrrolopyridyl (such as 2,3-dihydro-1H-pyrrolo[3,4-c]pyridyl), 6,7-dihydro-4H-pyrazolo[5,1-c][1,4]oxazinyl, 1,2,3,4-tetrahydro-1,5-naphthyridinyl, benzindazolyl (such as 1H-benzo[f]indazolyl), tetrahydrobenzindazolyl (such as 5,6,7,8-tetrahydro-1H-benzo[f]indazolyl), pyrazoloquinolyl (such as 1H-pyrazolo[4,3-g]quinolyl), tetrahydrocyclopentaindazolyl (such as 1,5,6,7-tetrahydrocyclopenta[f]indazolyl), dihydrocyclopentaindazolyl (such as 1,7-dihydrocyclopenta[f]indazolyl), dihydroindenothiazolyl (such as 6,7-dihydro-5H-indeno[5,6-d]thiazolyl), hexahydroindenoxazinyl (such as 2,3,4,6,7,8-hexahydroindeno[5,6-b][1,4]oxazinyl), tetrahydrocyclopentaindolyl (such as 1,5,6,7-tetrahydrocyclopenta[f]indolyl), tetrahydroindenoimidazolyl (such as 1,5,6,7-tetrahydroindeno[5,6-d]imidazolyl), preferably indazolyl, indolyl, benzimidazolyl, benzothiazolyl, quinolyl, 2-oxoquinolyl, 1,2,3,4-tetrahydroquinolyl, isoquinolinyl, 5,6,7,8-tetrahydroisoquinolyl, 1,2,3,4-tetrahydro-2,7-naphthyridinyl, 6,7-dihydro-5H-cyclopenta[c]pyridyl, 2,3-dihydro-1H-pyrrolo[3,4-c]pyridyl, imidazo[1,2-c]pyrimidinyl, imidazo[1,2-a]pyridyl, 1H-pyrrolo[2,3-b]pyridyl, [1,2,4]triazolo[1,5-a]pyridyl, 1,2,3,4-tetrahydro-1,5-naphthyridinyl, 1H-benzo[f]indazolyl, 5,6,7,8-tetrahydro-1H-benzo[f]indazolyl, 1H-pyrazolo[4,3-g]quinolyl, 1,5,6,7-tetrahydrocyclopenta[f]indazolyl, 1,7-dihydrocyclopenta[f]indazolyl, 6,7-dihydro-5H-indeno[5,6-d]thiazolyl, 2,3,4,6,7,8-hexahydroindeno[5,6-b][1,4]oxazinyl), 1,5,6,7-tetrahydrocyclopenta[f]indolyl, 1,5,6,7-tetrahydroindeno[5,6-d]imidazolyl.

The term “—OH” as used herein refers to hydroxyl radical.

The term “—CN” as used herein refers to cyano radical.

The term “oxo” as used herein refers to ═O.

The term “optional” or “optionally” as used herein means that the subsequently described event or circumstance may or may not occur, and the description includes instances wherein the event or circumstance occur and instances in which it does not occur. For example, “optionally substituted with one or more” includes unsubstituted and substituted with one or more substituents as described. It will be understood by those skilled in the art, with respect to any group containing one or more substituents, that such groups are not intended to introduce any substitution or substitution patterns that are sterically impractical, chemically incorrect, synthetically non-feasible and/or inherently unstable.

The term “substituted” or “substituted with . . . ”, as used herein, means that one or more (such as, 1, 2, 3 or 4) hydrogens on the designated atom or group are replaced with one or more (such as 1, 2, 3 or 4) substituents, preferably the substituents selected from the indicated group of substituents or radicals, provided that the designated atom's normal valence is not exceeded. The said substituents may be the same or different from each other. The term “substituted with one or more groups selected from . . . ” or “substituted with one or more . . . ” as used herein means that one or more hydrogens on the designated atom or group are independently replaced with one or more radicals from the indicated group of substituents or radicals, wherein the said radicals may be the same or different from each other. Preferably, “substituted with one or more groups selected from . . . ” or “substituted with one or more . . . ” means that the designated atom or group is substituted with 1, 2, 3, or 4 radicals independently selected from the indicated group of substituents or radicals, wherein the said radicals may be the same or different from each other. In some embodiments, when a substituent is oxo (i.e., ═O), then 2 hydrogens on a single atom are replaced by the oxo. An optional substituent can be any radicals, provided that combinations of substituents and/or variables result in a chemically correct and stable compound. A chemically correct and stable compound is meant to imply a compound that is sufficiently robust to survive sufficient isolation from a reaction mixture to be able to identify the chemical structure of the compound. Preferably, substituents are those exemplified in the compounds of the examples of the present application.

Unless otherwise specified, substituents are named into the core structure. For example, it is to be understood that when (cycloalkyl)alkyl is listed as a possible substituent, the point of attachment of this substituent to the core structure is in the alkyl portion.

When a structural formula herein contains an asterisk “*”, it means that the chiral center at the “*” mark in the compound is a single configuration of (R) configuration or (S) configuration; wherein the content of the single-configuration compound marked with “*” is at least 90% (e.g., 90%, 91%, 92%, 93%, 94%, 95%, 96%, 97%, 98%, 99%, 99.5%, 99.9%, 100%, or any value between these listed values). For example, some compounds of the present invention, such as the following compound of formula (a), and its structural formula contains an asterisk “*”, which means that the compound is a compound of formula (b) or compound of formula (c) in a single configuration.

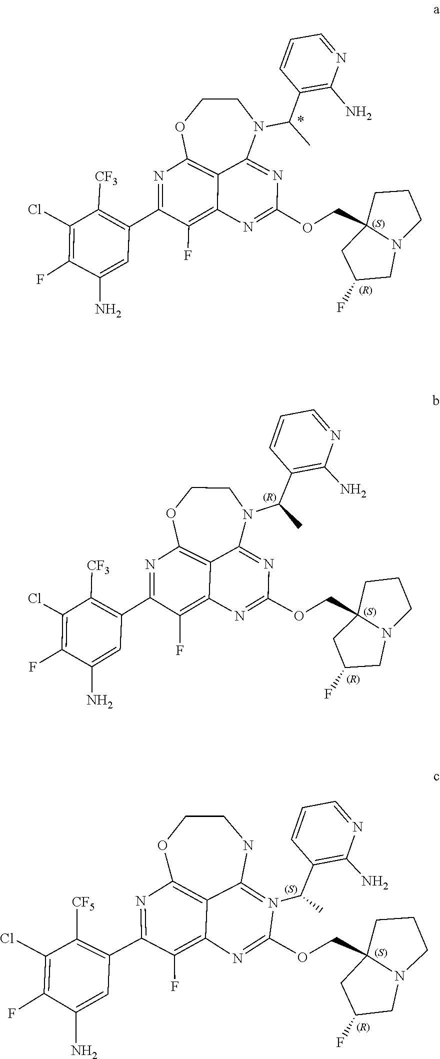

It will be appreciated by the person of ordinary skill in the art (“POSITA”) that some of the compounds of formula (I) may contain one or more chiral centers and therefore exist in two or more stereoisomeric forms. The racemates of these isomers, the individual isomers and mixtures enriched in one enantiomer, as well as diastereomers when there are two chiral centers, and mixtures partially enriched with specific diastereomers are within the scope of the present invention. It will be further appreciated by the POSITA that the present invention includes all the individual stereoisomers (e.g. enantiomers, diastereomers), racemic mixtures or partially resolved mixtures of the compounds of formula (I) and, where appropriate, the individual tautomeric forms thereof.

The term “stereoisomers” as used herein refers to compounds that have the same chemical constitution but differ in the arrangement of atoms or groups in space. Stercoisomers include enantiomers, diastereomers and the like.

The terms “enantiomers” and “enantiomeric forms” as used herein can be used interchangeably and refer to two stereoisomers of a compound that are non-superimposable mirror images of each other.

The terms “diastereomers” and “diastereomeric forms” as used herein can be used interchangeably and refer to stereoisomers that have two or more chiral centers and whose molecules are not mirror images of each other. Diastereomers have different physical properties, such as melting points, boiling points, spectral properties, or biological activities. A mixture of diastereomers can be separated by high-resolution analytical methods such as electrophoresis and chromatography such as HPLC.

In some embodiments, the present invention provides compounds of various stereoisomeric purities, that is, enantiomeric or diastereomeric purity expressed in different “ee” or “de” values. In some embodiments, the compound of formula (I) described herein has an enantiomeric purity of at least 60% ee (e.g., 60%, 65%, 70%, 75%, 80%, 85%, 90%, 91%, 92%, 93%, 94%, 95%, 96%, 97%, 98%, 99%, 99.5%, 990.9%, 100% ee, or any value between these listed values). In some embodiments, the compound of formula (I) described herein has an enantiomeric purity of greater than 99.9% ee. In some embodiments, the compound of formula (I) described herein has a diastereomeric purity of at least 60% de (e.g., 60%, 65%, 70%, 75%, 80%, 85%, 90%, 91%, 92%, 93%, 94%, 95%, 96%, 97%, 98%, 99%, 99.5%, 99.9%, 100% de, or any value between these listed values). In some embodiments, the compound of formula (I) described herein has a diastereomeric purity of greater than 99.9% de.

The term “enantiomeric excess” or “ee” refers to the amount of one enantiomer relative to the other. For a mixture of R and S enantiomers, the percentage of enantiomeric excess is defined as |R−S|*100, where R and S are the mole or weight fractions of the respective enantiomers in the mixture, R+S=1. If the optical rotation of a chiral substance is known, the percentage of enantiomeric excess is defined as ([a]obs/[a]max)*100, wherein [a]obs is the optical rotation of the enantiomeric mixture, and [a]max is the optical rotation of the pure enantiomer.

The term “diastereomeric excess” or “de” refers to the amount of one diastereomer relative to the other, and is defined by analogy based on the enantiomeric excess. Therefore, for a mixture of diastereomers D1 and D2, the percentage of diastereomeric excess is defined as |D1−D2|*100, wherein D1 and D2 are the mole or weight fractions of the respective diastereomers in the mixture, D1+D2=1.

The diastereomeric excess and enantiomeric excess can be measured by a number of analytical techniques (including nuclear magnetic resonance spectroscopy, chiral column chromatography and/or optical polarimetry) according to conventional protocols well known to a person skilled in the art.

The racemates can be used as such or can be resolved into their individual isomers. The resolution can afford stereochemically pure compounds or mixtures enriched in one or more isomers. Methods for separation of isomers are well known (cf. Allinger N. L. and Eliel E. L. in “Topics in Stereochemistry”, Vol. 6, Wiley Interscience, 1971) and include physical methods such as chromatography using a chiral adsorbent. Individual isomers can be prepared in chiral form from chiral precursors. Alternatively, individual isomers can be separated chemically from a mixture by: forming diastereomeric salts with a chiral acid (such as the individual enantiomers of 10-camphorsulfonic acid, camphoric acid, alpha-bromocamphoric acid, tartaric acid, diacetyltartaric acid, malic acid, pyrrolidone-5-carboxylic acid, and the like), fractionally crystallizing the salts, and then freeing one or both of the resolved bases, optionally repeating the process, so as obtain either or both substantially free of the other; i.e., in a form having an optical purity of >95%. Alternatively, the racemates can be covalently linked to a chiral compound (auxiliary) to produce diastereomers which can be separated by chromatography or by fractional crystallization after which time the chiral auxiliary is chemically removed to afford the pure enantiomers.

The term “tautomer” as used herein refers to constitutional isomers of compounds generated by rapid movement of an atom in two positions in a molecule. Tautomers readily interconvert into each other, e.g., enol form and ketone form are tipical tautomers.

A “pharmaceutically acceptable salt” is intended to mean a salt of a free acid or base of a compound of Formula (I) that is non-toxic, biologically tolerable, or otherwise biologically suitable for administration to the subject to be treated or prevented. For example, an acid addition salt includes such as a salt derived from an inorganic acid and an organic acid. For examples, see, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002.

In addition, if a compound described herein is obtained as an acid addition salt, the free base can be obtained by basifying a solution of the acid addition salt. Conversely, if the product is a free base, an acid addition salt, particularly a pharmaceutically acceptable acid addition salt, may be produced by dissolving the free base in a suitable solvent and treating the solution with an acid, in accordance with conventional procedures for preparing acid addition salts from base compounds. The POSITA will recognize various synthetic methodologies that may be used without undue experimentation to prepare non-toxic pharmaceutically acceptable acid addition salts or base addition salts.

The term “solvate” means solvent addition forms that contain either stoichiometric or non-stoichiometric amounts of solvent. Some compounds have a tendency to trap a fixed molar ratio of solvent molecules in the solid state, thus forming a solvate. If the solvent is water, the solvate formed is a hydrate, when the solvent is alcohol, the solvate formed is an alcoholate. Hydrates are formed by the combination of one or more molecules of water, or less than one molecule of water, with one molecule of the substances in which the water retains its molecular state as H₂O, such combination being able to form one or more hydrates, for example, hemihydrate, monohydrate, and dihydrate.

The compounds of the present invention also embrace isotopically-labeled compounds that one or more atoms are replaced by an atom having an atomic mass or mass number different from the atomic mass or mass number usually found in nature. All isotopes of any particular atom or element as specified are contemplated herein, and their uses. Exemplary isotopes that can be incorporated into compounds of the present invention include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵N, ¹⁵O, ¹⁷O, ¹⁸O, ³²P, ³³P ³⁵S, ¹⁸F, ³⁶Cl, ¹²³I, and ¹²⁵I. Certain isotopicaliy-labeled compounds of the present invention (e.g., those labeled with ³H and ¹⁴C) are useful in compound and/or substrate tissue distribution assays. Tritium (i.e., ³H) and carbon-14 (i.e., ¹⁴C) isotopes are particularly useful for this purpose in view of their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. It will be appreciated that the hydrogen (¹H) atom present in the compound of formula (I) described herein can be replaced with a deuterium (²H) atom to give the deuterated compound. In any given compound of formula (I), any number of hydrogen atoms may be replaced by the same number of deuterium atoms. Isotopically labeled compounds of formula (I) can generally be prepared by conventional techniques known to those skilled in the art or by processes analogous to those described in the examples as set out below using an appropriate isotopicaily-labeled reagent in place of the non-labeled reagent.

As used herein, the terms “group(s)” and “radical(s)” are synonymous and are intended to indicate functional groups or fragments of molecules attachable to other fragments of molecules.

The term “active ingredient” is used to indicate a chemical substance which has biological activity. In some embodiments, an “active ingredient” is a chemical substance having pharmaceutical utility.

The term “pharmaceutical combination” as used herein means a product obtained by mixing or combining two or more active ingredients, including fixed and non-fixed combinations of active ingredients, such as a kit, and a pharmaceutical composition. The term “fixed combination” means that two or more active ingredients (such as compounds of the present invention and additional therapeutic agents) are administered simultaneously to a patient in the form of a single entity or dose. The term “non-fixed combination” means that two or more active ingredients (such as compounds of the present invention and additional therapeutic agents) are administered simultaneously, in parallel or successively to a patient in separate entities, wherein the administration provides the patient with a therapeutically effective level of the compound.

The terms “treating” or “treatment” or “prevention” of a disease or disorder, in the context of achieving therapeutic benefit, refer to administering one or more pharmaceutical substances, especially compounds of the present invention to a subject that has the disease or disorder, or has a symptom of a disease or disorder, or has a predisposition toward a disease or disorder, with the purpose to cure, heal, alleviate, relieve, alter, remedy, ameliorate, improve, or affect the disease or disorder, the symptoms of the disease or disorder, or the predisposition toward the disease or disorder.

In some embodiments, the disease or disorder is cancer, such as solid tumors or hematologic malignancies.

The terms “treating”, “contacting” and “reacting,” in the context of a chemical reaction, mean adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately lead to the formation of the indicated and/or the desired product.

The term “effective amount” as used herein refers to an amount or dose of a KRAS inhibitor sufficient to generally bring about a therapeutic benefit in patients in need of treatment or prevention for a disease or disorder containing KRAS mutations or KRAS gene amplification. Effective amounts or doses of the active ingredient of the present disclosure may be ascertained by methods such as modeling, dose escalation studies or clinical trials, and by taking into consideration factors, e.g., the mode or route of administration or drug delivery, the pharmacokinetics of the agent, the severity and course of the disease or disorder, the subject's previous or ongoing therapy, the subject's health status and response to drugs, and the judgment of the attending physician.

An exemplary dose is in the range of from about 0.0001 to about 200 mg of active agent per kg of subject's body weight per day. such as from about 0.001 to 100 mg/kg/day, or about 0.01 to 35 mg/kg/day, or about 0.1 to 10 mg/kg daily in single or divided dosage units (e.g., BID, TID, QID). For a 70-kg human, an illustrative range for a suitable dosage amount is from about 0.05 to about 7 g/day, or about 0.2 to about 5 g/day.

Once improvement of the patient's disease or disorder has occurred, the dose may be adjusted for maintenance treatment. For example, the dosage or the frequency of administration, or both, may be reduced as a function of the symptoms, to a level at which the desired therapeutic effect is maintained. Of course, if symptoms have been alleviated to an appropriate level, treatment may cease. Patients may, however, require intermittent treatment on a long-term basis upon any recurrence of symptoms.

The term “inhibition” or “inhibiting” indicates a decrease in the baseline activity of a biological activity or process. The term “inhibition of the activity of mutant KRAS proteins” is a practical pharmaceutical activity for purposes of this disclosure and refers to a decrease in the activity of mutant KRAS proteins as a direct or indirect response to the presence of the compound of the present invention, relative to the activity of mutant KRAS proteins in the absence of the compound of the present invention. The decrease in activity may be due to the direct interaction of the compound of the present invention with mutant KRAS proteins, or due to the interaction of the compound of the present invention, with one or more other factors that in turn affect the activity of mutant KRAS proteins. For example, the presence of the compound of the present invention may decrease the activity of mutant KRAS proteins by directly binding to the mutant KRAS proteins, by causing (directly or indirectly) another factor to decrease the activity of mutant KRAS proteins, or by (directly or indirectly) decreasing the amount of mutant KRAS proteins present in the cell or organism.

The term “subject” or “patient” as used herein means mammals and non-mammals. Mammals means any member of the mammalia class including, but not limited to, humans; non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” or “patient” does not denote a particular age or sex. In some embodiments, the subject or patient is a human.

In general, the term “about” is used herein to modify a numerical value above or below the stated value by a variance of 20%.

Technical and scientific terms used herein and not specifically defined have the meaning commonly understood by the POSITA to which the present disclosure pertains.

All numerical ranges herein shall be interpreted as disclosing each numerical value and subset of numerical values within the range, regardless of whether they are specifically otherwise disclosed. For example, when referring to any range of values, it should be regarded as referring to every value within the range of values, for example, every integer within the range of values. For example, C₁₋₆ as used herein represents the inclusion of 1, 2, 3, 4, 5 or 6 C. The invention relates to all values falling within the ranges, all smaller ranges and the upper or lower limits of the numerical range.

### Detailed Description of Embodiments

Embodiment 1. A compound of formula (I):

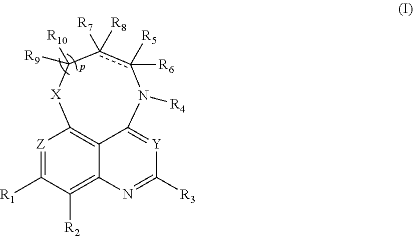

or a pharmaceutically acceptable salt thereof, or a solvate, a racemic mixture, an enantiomer, a diastereomer or a tautomer thereof, wherein
X is O, NR₁, CR₁₅R₁₋₆, C(O), S, S(O) or S(O)₂;
R₁₁ is independently selected from hydrogen, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, —CO(C₁₋₆ alkyl), —CONH₂, —CONH(C₁₋₆ alkyl), —CON(C₁₋₆ alkyl)₂, C₃₋₈ cycloalkyl, and 3-8 membered heterocyclyl;
R₁₅ and R₁₆ are each independently selected from hydrogen, deuterium, halogen, —CN, —OH, —NH₂, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O(C₁₋₆ alkyl), —O(C₁₋₆ haloalkyl), —NH(C₁₋₆ alkyl), —N(C₁₋₆ alkyl)₂, —CO(C₁₋₆ alkyl), —CONH₂, —CONH(C₁₋₆ alkyl), —CON(C₁₋₆ alkyl)₂, C₃₋₈ cycloalkyl, and 3-8 membered heterocyclyl; or R₁₅ and R₁₋₆ together with the carbon atom to which they are attached form C₃₋₆ cycloalkyl or 3-6 membered heterocyclyl;
Y is N, and Z is N; or Y is C—CN, and Z is N or Z is CR₂;
[symbol] represents a double bond or a single bond, and when [symbol] represents a double bond, R₆ and R₈ are absent; [symbol]
p is 0, 1 or 2;
R₁ is —L₁—R₁₈;
L₁ is absent, or L₁ is NR₁₁, O, C(O), S, S(O) or S(O)₂;
R₁₈ is selected from 6-14 membered aryl, 5-14 membered heteroaryl, C₃₋₁₀ cycloalkyl, and 4-14 membered heterocyclyl, each of which is optionally substituted with one or more groups independently selected from: halogen, —CN, —OH, —SH, —SF₅, oxo, C₁₋₆ alkyl, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆haloalkyl), —O(C₃₋₈ cycloalkyl), —O(3-8 membered heterocyclyl), —S(C₁₋₆ alkyl), —S(C₁₋₆ haloalkyl), —S(C₃₋₈ cycloalkyl), —S(3-8 membered heterocyclyl), —OC(O)NRₐR_{b}, —NRₐC(O)OR_{c}, —NRₐC(O)R_{d}, —NRₐR_{b}, —C(O)NRₐR_{b}, —C(O)OR_{c}, —C(O)R_{d}, —S(O)₂R_{f}, and —S(O)₂NRₐR_{b}; wherein the C₁₋₆ alkyl is optionally substituted with one or more deuterium, and the C₂₋₆ alkenyl, C₂₋₆ alkynyl, C₃₋₈ cycloalkyl and 3-8 membered heterocyclyl are each optionally substituted with one or more halogen;
R₂ is selected from hydrogen, halogen, —CN, —OH, —SH, oxo, C₁₋₆ alkyl, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —O(C₃₋₈ cycloalkyl), —O(3-8 membered heterocyclyl), —S(C₁₋₆ alkyl), —S(C₁₋₆ haloalkyl), —S(C₃₋₈ cycloalkyl), —S(3-8 membered heterocyclyl), —OC(O)NRₐR_{b}, —NRₐC(O)OR_{c}, —NRₐC(O)R_{d}, —NRₐR_{b}, —C(O)NRₐR_{b}, —C(O)ORₑ, —C(O)R_{d}, —S(O)₂R_{f}, and —S(O)₂NRₐR_{b}; wherein the C₁₋₆ alkyl is optionally substituted with one or more deuterium, and the C₂₋₆ alkenyl, C₂₋₆ alkynyl, C₃₋₈ cycloalkyl and 3-8 membered heterocyclyl are each optionally substituted with one or more halogen;
R₃ is selected from hydrogen, —OH, —SH, —NH₂, C₁₋₆ alkyl, C₁₋₆ haloalkyl, —O(C₁₋₆ alkyl), —O(C₁₋₆ haloalkyl), —S(C₁₋₆ alkyl), —S(C₁₋₆ haloalkyl), —NH(C₁₋₆ alkyl), —N(C₁₋₆ alkyl)₂, C₃₋₈ cycloalkyl, 4-10 membered heterocyclyl, and —O—L₂-R₁₂; wherein the C₃₋₈ cycloalkyl and 4-10 membered heterocyclyl are each optionally substituted with one or more groups independently selected from: deuterium, halogen, —CN, —OH, —SH, oxo, —NH₂, C₁₋₆ alkyl, C₁₋₆ alkylidene, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), and —S(C₁₋₆ alkyl), wherein the C₁₋₆ alkylidene is optionally substituted with one or more halogen;
L₂ is absent, or L₂ is C₁₋₆ alkylene or C₃₋₈ cycloalkylene, wherein the C₁₋₆ alkylene and C₃₋₈ cycloalkylene are each optionally substituted with one or more deuterium or halogen;
R₁₂ is C₃₋₁₀ cycloalkyl or 4-12 membered heterocyclyl, each of which is optionally substituted with one or more groups independently selected from: deuterium, halogen, —CN, —OH, —SH, oxo, C₁₋₆ alkyl, C₁₋₆ alkylidene, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-O—(C₁₋₆ alkyl), —O(C₁₋₆ alkyl), —O(C₁₋₆ haloalkyl), —O(C₃₋₈ cycloalkyl), —O(3-8 membered heterocyclyl), —S(C₁₋₆ alkyl), —S(C₁₋₆ haloalkyl), —S(C₃₋₈ cycloalkyl), —S(3-8 membered heterocyclyl), —OC(O)NRₐR_{b}, —NRₐC(O)OR_{c}, —NRₐC(O)R_{d}, —NRₐR_{b}, —C(O)NRₐR_{b}, —C(O)OR_{c}, —C(O)R_{d}, —S(O)₂R_{f}, and —S(O)₂NRₐR_{b}; wherein the C₁₋₆ alkyl, C₁₋₆ alkylidene, C₂₋₆ alkenyl, C₂₋₆ alkynyl, C₃₋₈ cycloalkyl and 3-8 membered heterocyclyl are each optionally substituted with one or more R₁₇;
R₁₇ is selected from deuterium, halogen, —CN, —OH, —SH, oxo, C₁₋₆ alkyl, C₁₋₆ alkylidene, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —S(C₁₋₆ alkyl), —S(C₁₋₆ haloalkyl), —NRₐR_{b}, —NHCO(C₁₋₆ alkyl), —CO(C₁₋₆ alkyl), —CONH₂, —CONH(C₁₋₆ alkyl), —CON(C₁₋₆ alkyl)₂, —S(O)₂(C₁₋₆ alkyl), —S(O)₂NH(C₁₋₆ alkyl), and —S(O)₂N(C₁₋₆ alkyl)₂; wherein the 3-8 membered heterocyclyl is optionally substituted with one or more groups independently selected from: deuterium, halogen, —CN, —OH, oxo, —NH₂, C₁₋₆ alkyl, C₁₋₆ alkylidene, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —S(C₁₋₆ alkyl), —S(C₁₋₆ haloalkyl), —CO(C₁₋₆ alkyl), and —S(O)₂(C₁₋₆ alkyl);
R₄ is selected from C₁₋₆ alkyl, C₂₋₆ alkenyl, C₃₋₁₀ cycloalkyl, 4-12 membered heterocyclyl and 5-12 membered heteroaryl, each of which is optionally substituted with one or more R₁₃;
R₁₃ is selected from deuterium, halogen, —CN, —OH, —SH, oxo, —NH₂, C₁₋₆ alkyl, C₂₋₆ alkenyl, C₂₋₆ alkynyl, C₁₋₆ haloalkyl, C₃₋₈ cycloalkyl, 3-8 membered heterocyclyl, 6-10 membered aryl, 5-10 membered heteroaryl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —S(C₁₋₆ alkyl), —S(C₁₋₆ haloalkyl), —NHCO(C₁₋₆ alkyl), —NH(C₁₋₆ alkyl), —N(C₁₋₆alkyl)₂, —CO(C₁₋₆ alkyl), —CONH₂, —CONH(C₁₋₆ alkyl), —CON(C₁₋₆ alkyl)₂, —S(O)₂(C₁₋₆ alkyl), —S(O)₂NH(C₁₋₆ alkyl), and —S(O)₂N(C₁₋₆ alkyl)₂; wherein the C₃₋₈ cycloalkyl, 3-8 membered heterocyclyl, 6-10 membered aryl and 5-10 membered heteroaryl are each optionally substituted with one or more R₁₄;
R₁₄ is independently selected from deuterium, halogen, —CN, —OH, —SH, oxo, C₁₋₆ alkyl, C₁₋₆ haloalkyl, C₃₋₈ cycloalkyl, 3-8 membered heterocyclyl, phenyl, 5-6 membered heteroaryl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —O(C₃₋₈ cycloalkyl), —O(3-8 membered heterocyclyl), —S(C₁₋₆ alkyl), —S(C₁₋₆ haloalkyl), —S(C₃₋₈ cycloalkyl), —S(3-8 membered heterocyclyl), —NRₐC(O)R_{d}, —NRₐR_{b}, —C(O)NRₐR_{b}, —C(O)OR_{c}, —C(O)R_{d}, —S(O)₂R_{f}, and —S(O)₂NRₐR_{b}; wherein the C₁₋₆ alkyl is optionally substituted with one or more deuterium, and the C₃₋₈ cycloalkyl, 3-8 membered heterocyclyl, phenyl and 5-6 membered heteroaryl are each optionally substituted with one or more halogen;
R₅, R₆, R₇, R₈, R₉ and R₁₀ are each independently selected from hydrogen, deuterium, halogen, —CN, —OH, —SH, oxo, —NH₂, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O(C₁₋₆ alkyl), —O(C₁₋₆ haloalkyl), —NH(C₁₋₆ alkyl), —N(C₁₋₆ alkyl)₂, —CO(C₁₋₆ alkyl), —CONH₂, —CONH(C₁₋₆ alkyl), —CON(C₁₋₆ alkyl)₂, C₃₋₈ cycloalkyl, and 3-8 membered heterocyclyl; or R₅ and R₆, R₇ and R₈, R₉ and R₁₀, R₅ and R₇, R₇ and R₉, or R₅ and R₉ together with the carbon atoms to which they are attached form C₃₋₆ cycloalkyl or 3-6 membered heterocyclyl;
or R₄ and R₅ together with the nitrogen atom and carbon atom to which they are attached form 5-12 membered heteroaryl, which is optionally substituted with one or more R₁₄;
or R₄ and R₅ together with the nitrogen atom and carbon atom to which they are attached form 4-10 membered heterocyclyl; wherein the 4-10 membered heterocyclyl is substituted with one or more groups independently selected from: C₃₋₈ cycloalkyl, 3-8 membered heterocyclyl, 6-10 membered aryl, and 5-10 membered heteroaryl; and the 4-10 membered heterocyclyl is further optionally substituted with one or more groups independently selected from: deuterium, halogen, —CN, —OH, —SH, oxo, —NH₂, C₁₋₆ alkyl, C₂₋₆ alkenyl, C₂₋₆ alkynyl, C₁₋₆ haloalkyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —S(C₁₋₆ alkyl), —S(C₁₋₆ haloalkyl), —NHCO(C₁₋₆ alkyl), —NH(C₁₋₆ alkyl), —N(C₁₋₆ alkyl)₂, —CO(C₁₋₆ alkyl), —CONH₂, —CONH(C₁₋₆ alkyl), —CON(C₁₋₆ alkyl)₂, —S(O)₂(C₁₋₆ alkyl), —S(O)₂NH(C₁₋₆ alkyl), and —S(O)₂N(C₁₋₆ alkyl)₂; wherein the C₃₋₈ cycloalkyl, 3-8 membered heterocyclyl, 6-10 membered aryl and 5-10 membered heteroaryl are each optionally substituted with one or more R₁₄; and
Rₐ, R_{b}, R_{c}, R_{d} and R_{f} are each independently selected from hydrogen, C₁₋₆ alkyl, C₃₋₈ cycloalkyl, 3-8 membered heterocyclyl, phenyl, and 5-6 membered heteroaryl; wherein the C₁₋₆ alkyl, C₃₋₈ cycloalkyl, 3-8 membered heterocyclyl, phenyl and 5-6 membered heteroaryl are each optionally substituted with one or more groups independently selected from: halogen, —CN, —OH, —O(C₁₋₆ alkyl), C₃₋₈ cycloalkyl, 3-8 membered heterocyclyl, —NR_{g}R_{b}, —C(O)NR_{g}Rₕ, and —NR_{g}C(O)Rᵢ; wherein R_{g}, Rₕ, and Rᵢ are each independently selected from hydrogen, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, —(C₁₋₆ alkyl)-NH₂, —(C₁₋₆ alkyl)-NH(C₁₋₆ alkyl), —(C₁₋₆ alkyl)-N(C₁₋₆ alkyl)₂, C₃₋₈ cycloalkyl, 3-8 membered heterocyclyl, phenyl, and 5-6 membered heteroaryl; or Rₐ and R_{b} together with the nitrogen atom to which they are attached form 3-8 membered heterocyclyl.

Embodiment 2. The compound or the pharmaceutically acceptable salt thereof, or the solvate, the racemic mixture, the enantiomer, the diastereomer or the tautomer thereof according to embodiment 1, wherein
X is O, NR₁₁, CR₁₅R₁₋₆, C(O), S, S(O) or S(O)₂;
R₁₁ is independently selected from hydrogen, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, —CO(C₁₋₆ alkyl), —CONH₂, —CONH(C₁₋₆ alkyl), —CON(C₁₋₆ alkyl)₂, C₃₋₈ cycloalkyl, and 3-8 membered heterocyclyl;
R₁₅ and R₁₋₆ are each independently selected from hydrogen, deuterium, halogen, —CN, —OH, —NH₂, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O(C₁₋₆ alkyl), —O(C₁₋₆ haloalkyl), —NH(C₁₋₆ alkyl), —N(C₁₋₆ alkyl)₂, —CO(C₁₋₆ alkyl), —CONH₂, —CONH(C₁₋₆ alkyl), —CON(C₁₋₆ alkyl)₂, C₃₋₈ cycloalkyl, and 3-8 membered heterocyclyl; or R₁₅ and R₁₋₆ together with the carbon atom to which they are attached form C₃₋₆ cycloalkyl or 3-6 membered heterocyclyl;
Y is N, and Z is N; or Y is C—CN, and Z is N or Z is CR₂;
[symbol] represents a double bond or a single bond, and when [symbol] represents a double bond, R₆ and R₈ are absent;
p is 0, 1 or 2;
R₁ is —L₁—R₁₈;
L₁ is absent, or L₁ is NR₁₁, O, C(O), S, S(O) or S(O)₂;
R₁₈ is selected from 6-12 membered aryl, 5-12 membered heteroaryl, C₃₋₁₀ cycloalkyl, and 4-12 membered heterocyclyl, each of which is optionally substituted with one or more groups independently selected from: halogen, —CN, —OH, —SH, oxo, C₁₋₆ alkyl, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —O(C₃₋₈ cycloalkyl), —O(3-8 membered heterocyclyl), —S(C₁₋₆ alkyl), —S(C₁₋₆ haloalkyl), —S(C₃₋₈ cycloalkyl), —S(3-8 membered heterocyclyl), —OC(O)NRₐR_{b}, —NRₐC(O)OR_{c}, —NRₐC(O)R_{d}, —NRₐR_{b}, —C(O)NRₐR_{b}, —C(O)OR_{c}, —C(O)R_{d}, —S(O)₂R_{f}, and —S(O)₂NRₐR_{b}; wherein the C₁₋₆ alkyl is optionally substituted with one or more deuterium, and the C₂₋₆ alkenyl, C₂₋₆ alkynyl, C₃₋₈ cycloalkyl and 3-8 membered heterocyclyl are each optionally substituted with one or more halogen;
R₂ is selected from hydrogen, halogen, —CN, —OH, —SH, oxo, C₁₋₆ alkyl, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —O(C₃₋₈ cycloalkyl), —O(3-8 membered heterocyclyl), —S(C₁₋₆ alkyl), —S(C₁₋₆ haloalkyl), —S(C₃₋₈ cycloalkyl), —S(3-8 membered heterocyclyl), —OC(O)NRₐR_{b}, —NRₐC(O)OR_{d}, —NRₐC(O)R_{d}, —NRₐR_{b}, —C(O)NRₐR_{b}, —C(O)OR_{c}, —C(O)R_{d}, —S(O)₂R_{f}, and —S(O)₂NRₐR_{b}; wherein the C₁₋₆ alkyl is optionally substituted with one or more deuterium, and the C₂₋₆ alkenyl, C₂₋₆ alkynyl, C₃₋₈ cycloalkyl and 3-8 membered heterocyclyl are each optionally substituted with one or more halogen;
R₃ is selected from hydrogen, —OH, —SH, —NH₂, C₁₋₆ alkyl, C₁₋₆ haloalkyl, —O(C₁₋₆ alkyl), —O(C₁₋₆ haloalkyl), —S(C₁₋₆ alkyl), —S(C₁₋₆ haloalkyl), —NH(C₁₋₆ alkyl), —N(C₁₋₆ alkyl)₂, C₃₋₈ cycloalkyl, 4-10 membered heterocyclyl, and —O—L₂-R₁₂; wherein the C₃₋₈ cycloalkyl and 4-10 membered heterocyclyl are each optionally substituted with one or more groups independently selected from: deuterium, halogen, —CN, —OH, —SH, oxo, —NH₂, C₁₋₆ alkyl, C₁₋₆ alkylidene, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), and —S(C₁₋₆ alkyl), wherein the C₁₋₆ alkylidene is optionally substituted with one or more halogen;
L₂ is absent, or L₂ is C₁₋₆ alkylene or C₃₋₈ cycloalkylene, wherein the C₁₋₆ alkylene and C₃₋₈ cycloalkylene are each optionally substituted with one or more deuterium or halogen;
R₁₂ is C₃₋₁₀ cycloalkyl or 4-12 membered heterocyclyl, each of which is optionally substituted with one or more groups independently selected from: deuterium, halogen, —CN, —OH, —SH, oxo, C₁₋₆ alkyl, C₁₋₆ alkylidene, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-O—(C₁₋₆ alkyl), —O(C₁₋₆ alkyl), —O(C₁₋₆ haloalkyl), —O(C₃₋₈ cycloalkyl), —O(3-8 membered heterocyclyl), —S(C₁₋₆ alkyl), —S(C₁₋₆ haloalkyl), —S(C₃₋₈ cycloalkyl), —S(3-8 membered heterocyclyl), —OC(O)NRₐR_{b}, —NRₐC(O)OR_{c}, —NRₐC(O)R_{d}, —NRₐR_{b}, —C(O)NRₐR_{b}, —C(O)OR_{c}, —C(O)R_{d}, —S(O)₂R_{f}, and —S(O)₂NRₐR_{b}; wherein the C₁₋₆ alkyl, C₁₋₆ alkylidene, C₂₋₆ alkenyl, C₂₋₆ alkynyl, C₃₋₈ cycloalkyl and 3-8 membered heterocyclyl are each optionally substituted with one or more R₁₇;
R₁₇ is selected from deuterium, halogen, —CN, —OH, —SH, oxo, C₁₋₆ alkyl, C₁₋₆ alkylidene, C₂₋₆ alkenyl, C₂₋₆ alkynyl, C₁₋₆, haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —S(C₁₋₆ alkyl), —S(C₁₋₆ haloalkyl), —NRₐR_{b}, —NHCO(C₁₋₆ alkyl), —CO(C₁₋₆ alkyl), —CONH₂, —CONH(C₁₋₆ alkyl), —CON(C₁₋₆ alkyl)₂, —S(O)₂(C₁₋₆ alkyl), —S(O)₂NH(C₁₋₆ alkyl), and —S(O)₂N(C₁₋₆ alkyl)₂;
R₄ is selected from C₁₋₆ alkyl, C₂₋₆ alkenyl, C₃₋₁₀ cycloalkyl, 4-12 membered heterocyclyl and 5-12 membered heteroaryl, each of which is optionally substituted with one or more R₁₃;
R₁₃ is selected from deuterium, halogen, —CN, —OH, —SH, oxo, —NH₂, C₁₋₆ alkyl, C₂₋₆ alkenyl, C₂₋₆ alkynyl, C₁₋₆ haloalkyl, C₃₋₈ cycloalkyl, 3-8 membered heterocyclyl, 6-10 membered aryl, 5-10 membered heteroaryl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —S(C₁₋₆ alkyl), —S(C₁₋₆ haloalkyl), —NHCO(C₁₋₆ alkyl), —NH(C₁₋₆ alkyl), —N(C₁₋₆ alkyl)₂, —CO(C₁₋₆ alkyl), —CONH₂, —CONH(C₁₋₆ alkyl), —CON(C₁₋₆ alkyl)₂, —S(O)₂(C₁₋₆ alkyl), —S(O)₂NH(C₁₋₆ alkyl), and —S(O)₂N(C₁₋₆ alkyl)₂; wherein the C₃₋₈ cycloalkyl, 3-8 membered heterocyclyl, 6-10 membered aryl and 5-10 membered heteroaryl are each optionally substituted with one or more R₁₄;
R₁₄ is independently selected from deuterium, halogen, —CN, —OH, —SH, oxo, C₁₋₆ alkyl, C₁₋₆ haloalkyl, C₃₋₈ cycloalkyl, 3-8 membered heterocyclyl, phenyl, 5-6 membered heteroaryl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —O(C₃₋₈ cycloalkyl), —O(3-8 membered heterocyclyl), —S(C₁₋₆ alkyl), —S(C₁₋₆ haloalkyl), —S(C₃₋₈ cycloalkyl), —S(3-8 membered heterocyclyl), —NHCO(C₁₋₆ alkyl), —NRₐR_{b}, —C(O)NRₐR_{b}, —C(O)OR_{c}, —C(O)R_{d}, —S(O)₂R_{f}, and —S(O)₂NRₐR_{b}; wherein the C₁₋₆ alkyl is optionally substituted with one or more deuterium, and the C₃₋₈ cycloalkyl, 3-8 membered heterocyclyl, phenyl and 5-6 membered heteroaryl are each optionally substituted with one or more halogen;
R₅, R₆, R₇, R₈, R₉ and R₁₀ are each independently selected from hydrogen, deuterium, halogen, —CN, —OH, —SH, oxo, —NH₂, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O(C₁₋₆ alkyl), —O(C₁₋₆ haloalkyl), —NH(C₁₋₆ alkyl), —N(C₁₋₆ alkyl)₂, —CO(C₁₋₆ alkyl), —CONH₂, —CONH(C₁₋₆ alkyl), —CON(C₁₋₆ alkyl)₂, C₃₋₈ cycloalkyl, and 3-8 membered heterocyclyl; or R₅ and R₆, R₇ and R₈, R₉ and R₁₀, R₅ and R₇, R₇ and R₉, or R₅ and R₉ together with the carbon atoms to which they are attached form C₃₋₆ cycloalkyl or 3-6 membered heterocyclyl;
or R₄ and R₅ together with the nitrogen atom and carbon atom to which they are attached form 5-12 membered heteroaryl, which is optionally substituted with one or more R₁₄;
or R₄ and R₅ together with the nitrogen atom and carbon atom to which they are attached form 4-10 membered heterocyclyl; wherein the 4-10 membered heterocyclyl is substituted with one or more groups independently selected from: C₃₋₈ cycloalkyl, 3-8 membered heterocyclyl, 6-10 membered aryl, and 5-10 membered heteroaryl; and the 4-10 membered heterocyclyl is further optionally substituted with one or more groups independently selected from: deuterium, halogen, —CN, —OH, —SH, oxo, —NH₂, C₁₋₆ alkyl, C₂₋₆ alkenyl, C₂₋₆ alkynyl, C₁₋₆ haloalkyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —S(C₁₋₆ alkyl), —S(C₁₋₆ haloalkyl), —NHCO(C₁₋₆ alkyl), —NH(C₁₋₆ alkyl), —N(C₁₋₆ alkyl)₂, —CO(C₁₋₆ alkyl), —CONH₂, —CONH(C₁₋₆ alkyl), —CON(C₁₋₆ alkyl)₂, —S(O)₂(C₁₋₆ alkyl), —S(O)₂NH(C₁₋₆ alkyl), and —S(O)₂N(C₁₋₆ alkyl)₂; wherein the C₃₋₈ cycloalkyl, 3-8 membered heterocyclyl, 6-10 membered aryl and 5-10 membered heteroaryl are each optionally substituted with one or more R₁₄; and
Rₐ, R_{b}, R_{c}, R_{d} and R_{f} are each independently selected from hydrogen, C₁₋₆ alkyl, C₃₋₈ cycloalkyl, 3-8 membered heterocyclyl, phenyl, and 5-6 membered heteroaryl; wherein the C₁₋₆ alkyl, C₃₋₈ cycloalkyl, 3-8 membered heterocyclyl, phenyl and 5-6 membered heteroaryl are each optionally substituted with one or more groups independently selected from: halogen, —CN, —OH, —O(C₁₋₆ alkyl), C₃₋₈ cycloalkyl, 3-8 membered heterocyclyl, —NR_{g}Rₕ, and —NR_{g}C(O)Rᵢ; wherein R_{g}, Rₕ, and Rᵢ are each independently selected from hydrogen, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, —(C₁₋₆ alkyl)-NH₂, —(C₁₋₆ alkyl)-NH(C₁₋₆ alkyl), —(C₁₋₆ alkyl)-N(C₁₋₆ alkyl)₂, C₃₋₈ cycloalkyl, 3-8 membered heterocyclyl, phenyl, and 5-6 membered heteroaryl.

Embodiment 3. The compound or the pharmaceutically acceptable salt thereof, or the solvate, the racemic mixture, the enantiomer, the diastereomer or the tautomer thereof according to embodiment 1 or 2, wherein X is O.

Embodiment 4. The compound or the pharmaceutically acceptable salt thereof, or the solvate, the racemic mixture, the enantiomer, the diastereomer or the tautomer thereof according to embodiment 1 or 2, wherein Y is N, and Z is N.

Embodiment 5. The compound or the pharmaceutically acceptable salt thereof, or the solvate, the racemic mixture, the enantiomer, the diastereomer or the tautomer thereof according to embodiment 1 or 2, wherein the compound is a compound of formula (I-1):

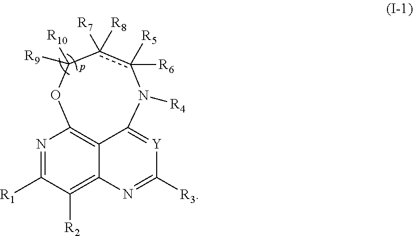

Embodiment 6. The compound or the pharmaceutically acceptable salt thereof, or the solvate, the racemic mixture, the enantiomer, the diastereomer or the tautomer thereof according to any one of embodiments 1-5, wherein p is 0 or 1; and preferably, p is 0.

Embodiment 7. The compound or the pharmaceutically acceptable salt thereof, or the solvate, the racemic mixture, the enantiomer, the diastereomer or the tautomer thereof according to any one of embodiments 1-6, wherein R₁ is —L₁—R₁₈; L₁ is absent, or L₁ is NR₁₁, wherein R₁₁ is independently selected from hydrogen, C₁₋₆ alkyl and C₁₋₆ haloalkyl; preferably, L₁ is absent, or L₁ is NH; and more preferably, L₁ is absent.

Embodiment 8. The compound or the pharmaceutically acceptable salt thereof, or the solvate, the racemic mixture, the enantiomer, the diastereomer or the tautomer thereof according to embodiment 7, wherein R₁₈ is selected from 6-10 membered aryl, 5-14 membered heteroaryl (such as 5-10 membered heteroaryl), C₃₋₈ cycloalkyl and 4-8 membered heterocyclyl, preferably selected from 6-10 membered aryl and 5-13 membered heteroaryl (such as 5-10 membered heteroaryl), more preferably 6-10 membered aryl and 8-13 membered heteroaryl, most preferably phenyl, naphthyl, 9-10 membered heteroaryl and 12-13 membered heteroaryl, each of which is optionally substituted with one or more groups independently selected from: halogen, —CN, —OH, —SH, —SF₅, oxo, C₁₋₆ alkyl, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆alkyl), —(C₁₋₆ alkyl)-CN, —O(C₁₋₆ alkyl), —O(C₁₋₆ haloalkyl), —OC(O)NRₐR_{b}, —NRₐC(O)OR_{c}, —NRₐC(O)R_{d}, —NRₐR_{b}, and —C(O)NRₐR_{b}; wherein the C₁₋₆ alkyl is optionally substituted with one or more deuterium, and the C₂₋₆ alkenyl, C₂₋₆ alkynyl, C₃₋₈ cycloalkyl and 3-8 membered heterocyclyl are each optionally substituted with one or more halogen.

Embodiment 9. The compound or the pharmaceutically acceptable salt thereof, or the solvate, the racemic mixture, the enantiomer, the diastereomer or the tautomer thereof according to embodiment 8, wherein R₁₈ is selected from phenyl, naphthyl, indanyl, benzocyclobutenyl, pyridyl, pyrimidinyl, pyrazinyl, indazolyl, indolyl, benzothiazolyl, benzothienyl, benzimidazolyl, benzindazolyl, tetrahydrobenzindazolyl, quinolyl, quinolinonyl, tetrahydroquinolyl, isoquinolyl, pyrazoloquinolyl, pyrrolopyridyl, triazolopyridyl, tetrahydrocyclopentaindazolyl, dihydrocyclopentaindazolyl, dihydroindenothiazolyl, hexahydroindenoxazinyl, tetrahydrocyclopentaindolyl, tetrahydroindenoimidazolyl, cyclohexyl, and cyclohexenyl, each of which is optionally substituted with one or more groups independently selected from: halogen, —CN, —OH, —SH, —SF₅, oxo, C₁₋₆ alkyl, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —OC(O)NRₐR_{b}, —NRₐC(O)R_{d}, —NRₐR_{b}, and —C(O)NRₐRe; wherein the C₁₋₆ alkyl is optionally substituted with one or more deuterium, and the C₂₋₆ alkenyl, C₂₋₆ alkynyl, C₃₋₈ cycloalkyl and 3-8 membered heterocyclyl are each optionally substituted with one or more halogen; preferably, R₁₈ is selected from phenyl, naphthyl, indanyl, benzocyclobutenyl, pyridyl, pyrimidinyl, pyrazinyl, indazolyl, benzothiazolyl, quinolyl, isoquinolyl, cyclohexyl, and cyclohexenyl, each of which is optionally substituted with one or more groups independently selected from: halogen, —CN, —OH, —SH, oxo, C₁₋₆ alkyl, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —NRₐC(O)R_{d}, and —NRₐR_{b}; wherein the C₁₋₆ alkyl is optionally substituted with one or more deuterium, and the C₂₋₆ alkenyl, C₂₋₆ alkynyl, C₃₋₈ cycloalkyl and 3-8 membered heterocyclyl are each optionally substituted with one or more halogen.

Embodiment 10. The compound or the pharmaceutically acceptable salt thereof, or the solvate, the racemic mixture, the enantiomer, the diastereomer or the tautomer thereof according to embodiment 9, wherein R₁₈ is selected from

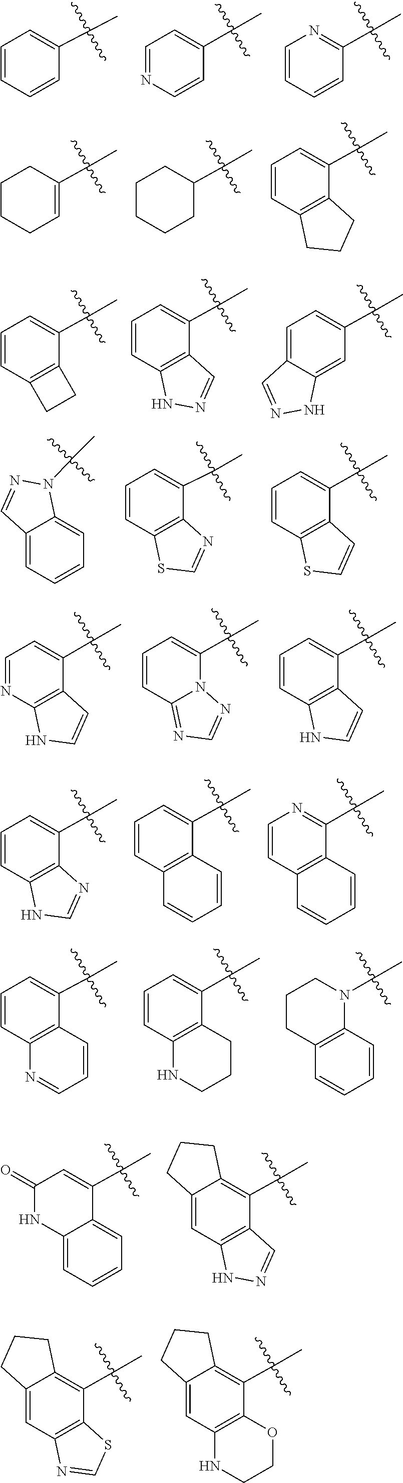

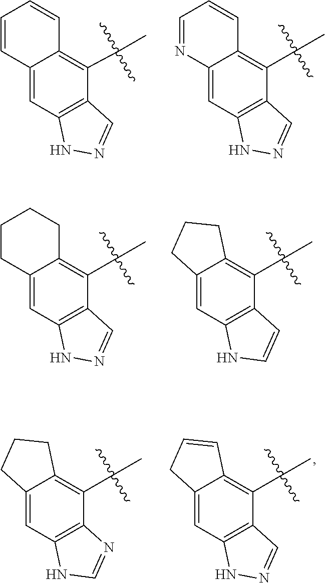 [SMILES: *c1c2ccccc2cc2[nH]ncc12 ; *c1c2cccnc2cc2[nH]ncc12 ; *c1c2c(cc3[nH]ncc13)CCCC2 ; *c1c2c(cc3[nH]ccc13)CCC2 ; *c1c2c(cc3[nH]cnc13)CCC2 ; *c1c2c(cc3[nH]ncc13)CC=C2]

each of which is optionally substituted with one or more groups independently selected from: halogen, —CN, —OH, C₁₋₆ alkyl, C₂₋₆ alkenyl, C₂₋₆ alkynyl, C₁₋₆ haloalkyl, C₃₋₈ cycloalkyl, —O(C₁₋₆ alkyl), —OC(O)NRₐR_{b}, —NRₐC(O)R_{d}, and —NRₐR_{b}; wherein Rₐ, R_{b} and R_{d} are each independently selected from hydrogen and C₁₋₆ alkyl; wherein the C₁₋₆ alkyl in Rₐ, R_{b} and R_{d} is optionally substituted with one or more groups independently selected from: —OH, —NR_{g}Rₕ, —C(O)NR_{g}Rₕ, and —NHC(O)Rᵢ; wherein R_{g}, Rₕ and Rᵢ are each independently selected from hydrogen, C₁₋₆ alkyl, and —(C₁₋₆ alkyl)-NH₂; or Rₐ and R_{b} together with the nitrogen atom to which they are attached form 3-6 membered heterocyclyl; preferably, R₁₈ is selected from

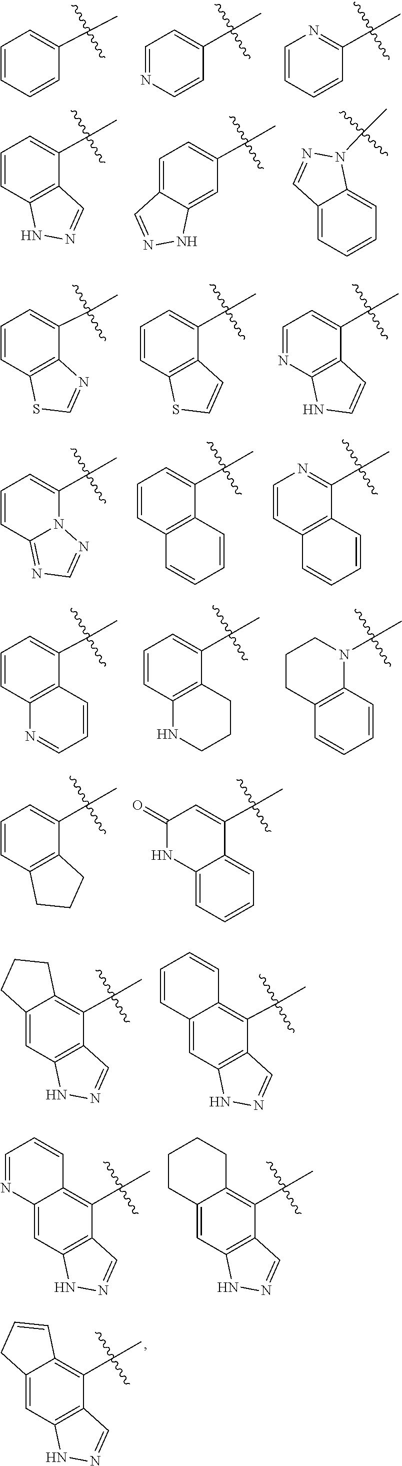

each of which is optionally substituted with one or more groups independently selected from: halogen, —OH, C₁₋₆ alkyl, C₂₋₆ alkynyl, C₁₋₆ haloalkyl, C₃₋₈ cycloalkyl, —O(C₁₋₆ alkyl), —OC(O)NRₐR_{b}, —NRₐC(O)R_{d}, and —NRₐR_{b}; wherein Rₐ, R_{b} and R_{d} are each independently selected from hydrogen and C₁₋₆ alkyl; wherein the C₁₋₆ alkyl in Rₐ, R_{b} and R_{d} is optionally substituted with one or more groups independently selected from: —OH, —NR_{g}R_{b}, —C(O)NR_{g}R₁₁, and —NHC(O)Rᵢ; wherein R_{g}, Rₕ and Rᵢ are each independently selected from hydrogen, C₁₋₆ alkyl, and —(C₁₋₆ alkyl)-NH₂; or Rₐ and R_{b} together with the nitrogen atom to which they are attached form 3-6 membered heterocyclyl;
more preferably, R₁₈ is selected from

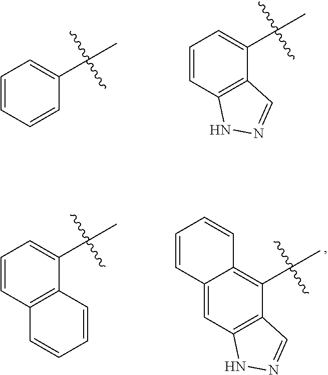 [SMILES: *c1ccccc1 ; *c1cccc2[nH]ncc12 ; *c1cccc2ccccc12 ; *c1c2ccccc2cc2[nH]ncc12]

each of which is optionally substituted with one or more groups independently selected from: halogen, —OH, C₁₋₆ alkyl, C₂₋₆ alkynyl, C₁₋₆ haloalkyl, C₃₋₈ cycloalkyl, —O(C₁₋₆ alkyl), —NRₐC(O)R_{d}, and —NRₐR_{b}; wherein Rₐ, R_{b} and R_{d} are each independently selected from hydrogen and C₁₋₆ alkyl; wherein the C₁₋₆ alkyl in Rₐ, R_{b} and R_{d} is optionally substituted with one or more groups independently selected from: —OH, —NR_{g}R_{b} and —NHC(O)R; wherein R_{g}, Rₕ, and Rᵢ are each independently selected from hydrogen, C₁₋₆ alkyl, and —(C₁₋₆ alkyl)-NH₂;
further preferably, R₁₈ is

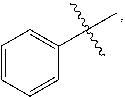 [SMILES: *c1ccccc1]

which is optionally substituted with one or more groups independently selected from: halogen, —OH, C₁₋₆ alkyl, C₂₋₆ alkynyl, C₁₋₆, haloalkyl, C₃₋₈ cycloalkyl, and —NH₂; or R₁₈ is

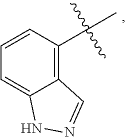 [SMILES: *c1cccc2[nH]ncc12]

which is optionally substituted with one or more groups independently selected from: halogen, C₁₋₆ alkyl, C₂₋₆ alkynyl, C₁₋₆ haloalkyl, C₃₋₈ cycloalkyl, and —NH₂; or R₁₈ is

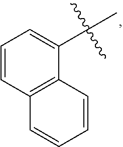 [SMILES: *c1cccc2ccccc12]

which is optionally substituted with one or more groups independently selected from: halogen, —OH, C₁₋₆ alkyl, and C₂₋₆ alkynyl; or R₁₈ is

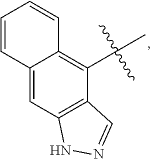 [SMILES: *c1c2ccccc2cc2[nH]ncc12]

which is optionally substituted with one or more groups independently selected from: halogen, C₁₋₆ alkyl, and C₃₋₈ cycloalkyl.

Embodiment 11. The compound or the pharmaceutically acceptable salt thereof, or the solvate, the racemic mixture, the enantiomer, the diastereomer or the tautomer thereof according to any one of embodiments 1-10, wherein R₂ is selected from hydrogen, halogen, —CN, —OH, —SH, C₁₋₆ alkyl, C₂₋₆ alkenyl, C₂₋₆ alkynyl, C₁₋₆ haloalkyl, C₃₋₈ cycloalkyl, —O(C₁₋₆ alkyl), and —NRₐR_{b}; wherein the C₁₋₆ alkyl is optionally substituted with one or more deuterium, and the C₂₋₆ alkenyl, C₂₋₆ alkynyl and C₃₋₈ cycloalkyl are each optionally substituted with one or more halogen; preferably, R₂ is selected from hydrogen, halogen, —CN, —OH, C₁₋₆ alkyl, C₂₋₆ alkynyl, C₁₋₆ haloalkyl, C₃₋₈ cycloalkyl, —O(C₁₋₆ alkyl), and —NH₂; and more preferably, R₂ is halogen, for example, F.

Embodiment 12. The compound or the pharmaceutically acceptable salt thereof, or the solvate, the racemic mixture, the enantiomer, the diastereomer or the tautomer thereof according to any one of embodiments 1-11, wherein R₃ is selected from hydrogen, —OH, —O(C₁₋₆ alkyl), —O(C₁₋₆ haloalkyl), —S(C₁₋₆ alkyl), —S(C₁₋₆ haloalkyl), C₃₋₈ cycloalkyl, 4-10 membered heterocyclyl, and —O—L₂-R₁₂; wherein the C₃₋₈ cycloalkyl and 4-10 membered heterocyclyl are each optionally substituted with one or more groups independently selected from: deuterium, halogen, —CN, —OH, oxo, C₁₋₆ alkyl, C₁₋₆ alkylidene, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, and —O(C₁₋₆ alkyl), wherein the C₁₋₆ alkylidene is optionally substituted with one or more halogen; preferably, R₃ is selected from hydrogen, —O(C₁₋₆ alkyl), —S(C₁₋₆ alkyl), 4-10 membered heterocyclyl, and —O—L₂-R₁₂; wherein the 4-10 membered heterocyclyl is optionally substituted with one or more groups independently selected from: deuterium, halogen, and —(C₁₋₆ alkyl)-OH; and more preferably, R₃ is —O—L₂-R₁₂.

Embodiment 13. The compound or the pharmaceutically acceptable salt thereof, or the solvate, the racemic mixture, the enantiomer, the diastereomer or the tautomer thereof according to embodiment 12, wherein L₂ is C₁₋₆ alkylene or C₃₋₈ cycloalkylene, each of which is optionally substituted with one or more deuterium or halogen; preferably, L₂ is C₁₋₆ alkylene, which is optionally substituted with one or more deuterium; and more preferably, L₂ is CH₂, which is optionally substituted with one or more deuterium.

Embodiment 14. The compound or the pharmaceutically acceptable salt thereof, or the solvate, the racemic mixture, the enantiomer, the diastereomer or the tautomer thereof according to either of embodiments 12-13, wherein R₁₂ is C₃₋₈ cycloalkyl or 4-10 membered heterocyclyl, preferably 4-10 membered heterocyclyl, more preferably 4-8 membered heterocyclyl, each of which is optionally substituted with one or more groups independently selected from: deuterium, halogen, —CN, —OH, oxo, C₁₋₆ alkyl, C₁₋₆ alkylidene, C₁₋₆ haloalkyl, C₃₋₈ cycloalkyl, 3-8 membered heterocyclyl, —O(C₁₋₆ alkyl), —O(C₁₋₆ haloalkyl), —NRₐC(O)R_{d}, —NRₐR_{b}, —C(O)NRₐR_{b}, —C(O)OR_{c}, and —C(O)R_{d}; wherein the C₁₋₆ alkyl, C₁₋₆ alkylidene, C₃₋₈ cycloalkyl and 3-8 membered heterocyclyl are each optionally substituted with one or more R₁₇.

Embodiment 15. The compound or the pharmaceutically acceptable salt thereof, or the solvate, the racemic mixture, the enantiomer, the diastereomer or the tautomer thereof according to embodiment 14, wherein R₁₂ is selected from cyclopropyl, cyclobutyl, azetidinyl, pyrrolidinyl, morpholinyl, piperidinyl, piperazinyl, tetrahydropyranyl, hexahydro-1H-pyrrolizinyl, tetrahydro-1′H,3′H-spiro[cyclopropane-1,2′-pyrrolizinyl]and octahydrocyclopropa[a]pyrrolizinyl, each of which is optionally substituted with one or more groups independently selected from: deuterium, halogen, —CN, —OH, oxo, C₁₋₆ alkyl, C₁₋₆ alkylidene, C₁₋₆ haloalkyl, C₃₋₈ cycloalkyl, 3-8 membered heterocyclyl, —O(C₁₋₆ alkyl), —O(C₁₋₆ haloalkyl), —NRₐC(O)R_{d}, —NRₐR_{b}, —C(O)NRₐR_{b}, —C(O)OR_{c}, and —C(O)R_{d}; wherein the C₁₋₆ alkyl, C₁₋₆ alkylidene, C₃₋₈ cycloalkyl and 3-8 membered heterocyclyl are each optionally substituted with one or more R₁₇.

Embodiment 16. The compound or the pharmaceutically acceptable salt thereof, or the solvate, the racemic mixture, the enantiomer, the diastereomer or the tautomer thereof according to embodiment 15, wherein R₁₂ is selected from

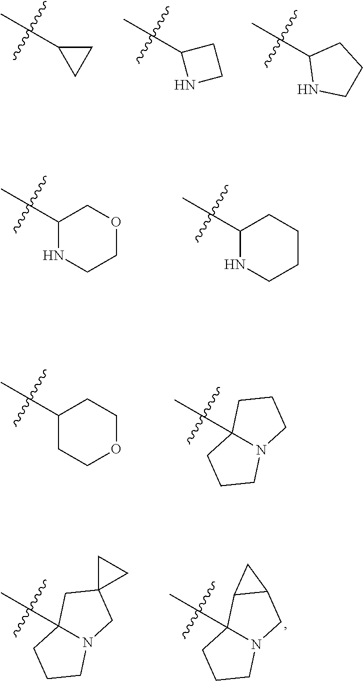

each of which is optionally substituted with one or more groups independently selected from: deuterium, halogen, —CN, —OH, oxo, C₁₋₆ alkyl, C₁₋₆ alkylidene, C₁₋₆ haloalkyl, C₃₋₈ cycloalkyl, 3-8 membered heterocyclyl, and —O(C₁₋₆ alkyl); wherein the C₁₋₆ alkyl, C₁₋₆ alkylidene, C₃₋₈ cycloalkyl and 3-8 membered heterocyclyl are each optionally substituted with one or more R₁₇; preferably, R₁₂ is selected from

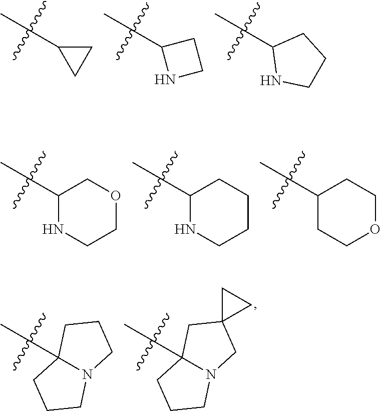

each of which is optionally substituted with one or more groups independently selected from: deuterium, halogen, —CN, oxo, C₁₋₆ alkyl, C₁₋₆ alkylidene, C₁₋₆ haloalkyl, and 3-8 membered heterocyclyl; wherein the C₁₋₆ alkyl, C₁₋₆ alkylidene and 3-8 membered heterocyclyl are each optionally substituted with one or more R₁₇;
more preferably, R₁₂ is selected from

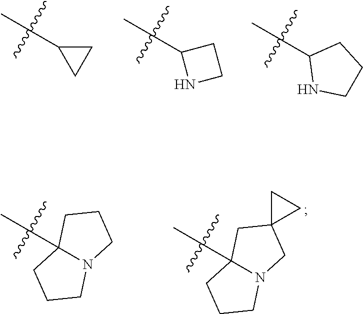

each of which is optionally substituted with one or more groups independently selected from: deuterium, halogen, —CN, oxo, C₁₋₆ alkyl, C₁₋₆ alkylidene, C₁₋₆ haloalkyl, and 3-8 membered heterocyclyl; wherein the C₁₋₆ alkyl, C₁₋₆ alkylidene and 3-8 membered heterocyclyl are each optionally substituted with one or more R₁₇;
further preferably, R₁₂ is

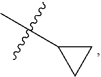 [SMILES: *C1CC1]

which is optionally substituted with one or more groups independently selected from: —CN and C₁₋₆ alkyl; wherein the C₁₋₆ alkyl is optionally substituted with one or more R₁₇; or R₁₂ is

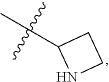 [SMILES: *C1CCN1]

which is optionally substituted with one or more groups independently selected from: C₁₋₆ haloalkyl; or R₁₂ is

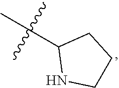 [SMILES: *C1CCCN1 ; ","]

which is optionally substituted with one or more groups independently selected from: deuterium, halogen, C₁₋₆ alkyl, C₁₋₆ haloalkyl, and 3-8 membered heterocyclyl; wherein the C₁₋₆ alkyl and 3-8 membered heterocyclyl are each optionally substituted with one or more R₁₇; or R₁₂ is

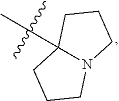 [SMILES: *C12CCCN1CCC2]

which is optionally substituted with one or more groups independently selected from: deuterium, halogen, and C₁₋₆ alkylidene; wherein the C₁₋₆ alkylidene is optionally substituted with one or more R₁₇.

Embodiment 17. The compound or the pharmaceutically acceptable salt thereof, or the solvate, the racemic mixture, the enantiomer, the diastereomer or the tautomer thereof according to any one of embodiments 14-16, wherein R₁₇ is selected from deuterium, halogen, —CN, —OH, oxo, 3-8 membered heterocyclyl, —O(C₁₋₆ alkyl), —O(C₁₋₆ haloalkyl), —NRₐR_{b}, and —NHCO(C₁₋₆ alkyl), wherein the 3-8 membered heterocyclyl is optionally substituted with one or more groups independently selected from: halogen, —CN, —OH, oxo, C₁₋₆ alkyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —CO(C₁₋₆ alkyl), and —S(O)₂(C₁₋₆ alkyl); preferably, R₁₇ is selected from deuterium, halogen, —OH, 3-8 membered heterocyclyl, and —NRₐR_{b}, wherein the 3-8 membered heterocyclyl is optionally substituted with one or more groups independently selected from: halogen, C₁₋₆ alkyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —CO(C₁₋₆ alkyl), and —S(O)₂(C₁₋₆ alkyl); more preferably, R₁₇ is selected from deuterium, halogen, —OH, 3-8 membered heterocyclyl, —N(C₁₋₆ alkyl)₂, and —N(C₁₋₆ alkyl)(C₃₋₈ cycloalkyl), wherein the C₁₋₆ alkyl is optionally substituted with one or more —OH, and the 3-8 membered heterocyclyl is optionally substituted with one or more groups independently selected from: C₃₋₈ cycloalkyl, 3-8 membered heterocyclyl, and —(C₁₋₆ alkyl)-OH; and further preferably, R₁₇ is selected from deuterium, and halogen, for example, F.

Embodiment 18. The compound or the pharmaceutically acceptable salt thereof, or the solvate, the racemic mixture, the enantiomer, the diastereomer or the tautomer thereof according to any one of embodiments 1-17, wherein R₄ is selected from C₁₋₆ alkyl, C₃₋₈ cycloalkyl, 4-8 membered heterocyclyl, and 5-10 membered heteroaryl, each of which is optionally substituted with one or more R₁₃; preferably, R₄ is selected from C₁₋₆ alkyl, C₃₋₈ cycloalkyl, and 4-8 membered heterocyclyl, each of which is optionally substituted with one or more R₁₃; more preferably, R₄ is selected from C₁₋₆ alkyl, and C₃₋₈ cycloalkyl, each of which is optionally substituted with one or more R₁₃; further preferably, R₄ is C₁₋₆ alkyl, which is optionally substituted with one or more R₁₃; and most preferably, R₄ is C₁₋₆ alkyl, which is substituted with one or more R₁₃.

Embodiment 19. The compound or the pharmaceutically acceptable salt thereof, or the solvate, the racemic mixture, the enantiomer, the diastereomer or the tautomer thereof according to embodiment 18, wherein R₁₃ is selected from deuterium, halogen, —OH, oxo, —NH₂, C₃₋₈ cycloalkyl, 3-8 membered heterocyclyl, 6-10 membered aryl, 5-10 membered heteroaryl, —O(C₁₋₆ alkyl), —NHCO(C₁₋₆ alkyl), —NH(C₁₋₆ alkyl), and —N(C₁₋₆ alkyl)₂; wherein the C₃₋₈ cycloalkyl, 3-8 membered heterocyclyl, 6-10 membered aryl and 5-10 membered heteroaryl are each optionally substituted with one or more R₁₄; preferably, R₁₃ is selected from deuterium, oxo, —NH₂, and 5-10 membered heteroaryl; wherein the 5-10 membered heteroaryl is optionally substituted with one or more R₁₄; more preferably, R₁₃ is selected from deuterium, and 5-10 membered heteroaryl; wherein the 5-10 membered heteroaryl is optionally substituted with one or more R₁₄; and further preferably, R₁₃ is 5-6 membered heteroaryl, which is optionally substituted with one or more R₁₄.

Embodiment 20. The compound or the pharmaceutically acceptable salt thereof, or the solvate, the racemic mixture, the enantiomer, the diastereomer or the tautomer thereof according to embodiment 19, wherein R₁₃ is selected from phenyl, pyrazolyl, pyridyl, pyridazinyl, pyrimidinyl, pyrazinyl, and imidazopyridyl, each of which is optionally substituted with one or more R₁₄.

Embodiment 21. The compound or the pharmaceutically acceptable salt thereof, or the solvate, the racemic mixture, the enantiomer, the diastereomer or the tautomer thereof according to embodiment 20, wherein R₁₃ is selected from

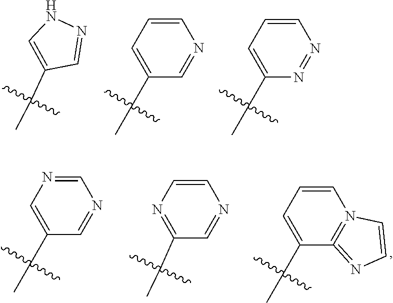

each of which is optionally substituted with one or more R₁₄;
preferably, R₁₃ is selected from

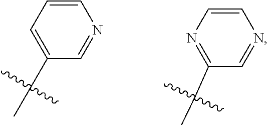 [SMILES: *c1cccnc1 ; *Cc1cnccn1]

each of which is optionally substituted with one or more R₁₄;
more preferably, R₁₃ is

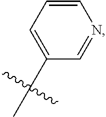 [SMILES: *c1cccnc1]

which is optionally substituted with one or more R₁₄; or R₁₃ is

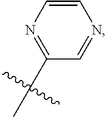 [SMILES: *c1cnccn1]

which is optionally substituted with one or more R₁₄.

Embodiment 22. The compound or the pharmaceutically acceptable salt thereof, or the solvate, the racemic mixture, the enantiomer, the diastereomer or the tautomer thereof according to any one of embodiments 19-21, wherein R₁₄ is independently selected from deuterium, halogen, —CN, —OH, oxo, C₁₋₆ alkyl, C₁₋₆ haloalkyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —NRₐC(O)Rₐ, —NRₐR_{b}, and —C(O)NRₐR_{b}; wherein the C₁₋₆ alkyl is optionally substituted with one or more deuterium; preferably, R₁₄ is independently selected from halogen and —NRₐR_{b}; and more preferably, R₁₄ is —NH₂.

Embodiment 23. The compound or the pharmaceutically acceptable salt thereof, or the solvate, the racemic mixture, the enantiomer, the diastereomer or the tautomer thereof according to any one of embodiments 1-22, wherein R₅, R₆, R₇, R₈, R₉ and R₁₀ are each independently selected from hydrogen, deuterium, C₁₋₆ alkyl, C₁₋₆ haloalkyl, and C₃₋₈ cycloalkyl; or R₅ and R₆, R₇ and R₈, R₉ and R₁₀, R₅ and R₇, R₇ and R₉, or R₅ and R₉ together with the carbon atoms to which they are attached form C₃₋₆ cycloalkyl or 3-6 membered heterocyclyl; preferably, R₅, R₆, R₇, R₈, R₉ and R₁₀ are each independently selected from hydrogen, deuterium and C₁₋₃ alkyl; or R₅ and R₆ together with the carbon atom to which they are attached form cyclopropyl; or R₅ and R₇ together with the carbon atoms to which they are attached form cyclopropyl, cyclopentyl, tetrahydrofuranyl, or tetrahydropyranyl; more preferably, R₅, R₆, R₇, R₈, R₉ and R₁₀ are all hydrogen; or R₅ is C₁₋₃ alkyl, and R₆, R₇, R₈, R₉ and R₁₀ are each independently selected from hydrogen and deuterium; or R₅ and R₇ together with the carbon atoms to which they are attached form cyclopentyl, tetrahydrofuranyl, or tetrahydropyranyl.

Embodiment 24. The compound or the pharmaceutically acceptable salt thereof, or the solvate, the racemic mixture, the enantiomer, the diastereomer or the tautomer thereof according to embodiment 1, wherein the compound is a compound of formula (I-2):

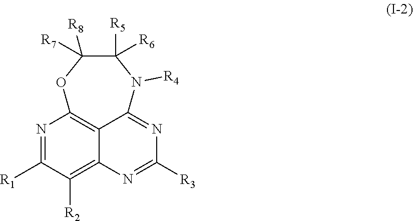

wherein:
R₁ is —L₁—R₁₈;
L₁ is absent;
R₁₈ is selected from 6-10 membered aryl and 5-14 membered heteroaryl, each of which is optionally substituted with one or more groups independently selected from: halogen, —CN, —OH, C₁₋₆ alkyl, C₂₋₆ alkenyl, C₂₋₆ alkynyl, C₁₋₆ haloalkyl, C₃₋₈ cycloalkyl, —O(C₁₋₆ alkyl), —OC(O)NRₐR_{b}, —NRₐC(O)R_{d}, and —NRₐR_{b}; preferably, R₁₈ is selected from

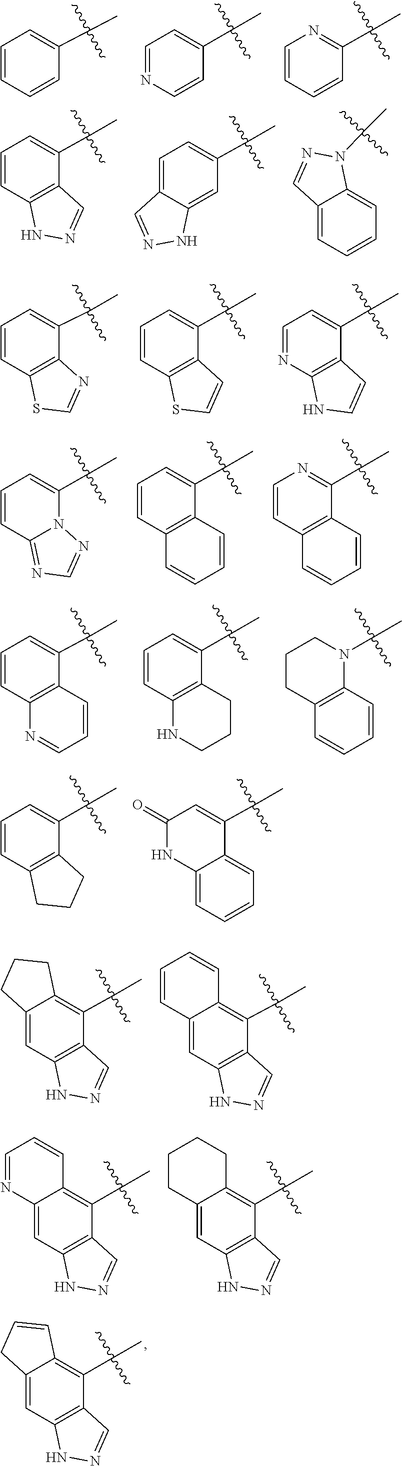

each of which is optionally substituted with one or more groups independently selected from: halogen, —OH, C₁₋₆ alkyl, C₂₋₆ alkynyl, C₁₋₆ haloalkyl, C₃₋₈ cycloalkyl, —O(C₁₋₆ alkyl), —OC(O)NRₐR_{b}, —NRₐC(O)Rₐ, and —NRₐR_{b}; more preferably, R₁₈ is selected from

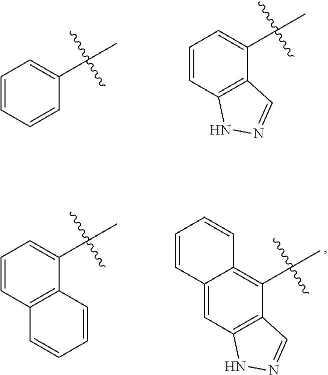 [SMILES: *c1ccccc1 ; *c1cccc2[nH]ncc12 ; *c1cccc2ccccc12 ; *c1c2ccccc2cc2[nH]ncc12]

each of which is optionally substituted with one or more groups independently selected from: halogen, —OH, C₁₋₆ alkyl, C₂₋₆ alkynyl, C₁₋₆ haloalkyl, C₃₋₈ cycloalkyl, —O(C₁₋₆ alkyl), —NRₐC(O)Rₐ, and —NRₐR_{b}; further preferably, R₁₈ is

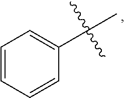 [SMILES: *c1ccccc1]

which is optionally substituted with one or more groups independently selected from: halogen, —OH, C₁₋₆ alkyl, C₂₋₆ alkynyl, C₁₋₆ haloalkyl, C₃₋₈ cycloalkyl, and —NH₂; or R₁₈ is

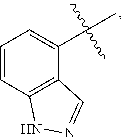 [SMILES: *c1cccc2[nH]ncc12]

which is optionally substituted with one or more groups independently selected from: halogen, C₁₋₆ alkyl, C₂₋₆ alkynyl, C₁₋₆ haloalkyl, C₃-8 cycloalkyl, and —NH₂; or R₁₈ is

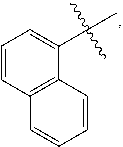 [SMILES: *c1cccc2ccccc12]

which is optionally substituted with one or more groups independently selected from: halogen, —OH, C₁₋₆ alkyl, and C₂₋₆ alkynyl; or R₁₈ is

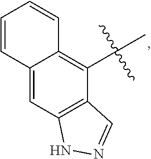 [SMILES: *c1c2ccccc2cc2[nH]ncc12]

which is optionally substituted with one or more groups independently selected from: halogen, C₁₋₆ alkyl, and C₃₋₈ cycloalkyl;
R₂ is selected from hydrogen, halogen, —CN, —OH, C₁₋₆ alkyl, C₂₋₆ alkynyl, C₁₋₆ haloalkyl, C₃₋₈ cycloalkyl, —O(C₁₋₆ alkyl), and —NH₂; and preferably, R₂ is halogen;
R₃ is —O—L₂—R₁₂;
L₂ is C₁₋₆ alkylene or C₃₋₈ cycloalkylene, each of which is optionally substituted with one or more deuterium or halogen; preferably, L₂ is C₁₋₆ alkylene, which is optionally substituted with one or more deuterium; and more preferably, L₂ is CH₂, which is optionally substituted with one or more deuterium;
R₁₂ is C₃₋₈ cycloalkyl or 4-10 membered heterocyclyl, each of which is optionally substituted with one or more groups independently selected from: deuterium, halogen, —CN, oxo, C₁₋₆ alkyl, C₁₋₆ alkylidene, C₁₋₆ haloalkyl, and 3-8 membered heterocyclyl; wherein the C₁₋₆ alkyl, C₁₋₆ alkylidene and 3-8 membered heterocyclyl are each optionally substituted with one or more R₁₇; preferably, R₁₂ is selected from

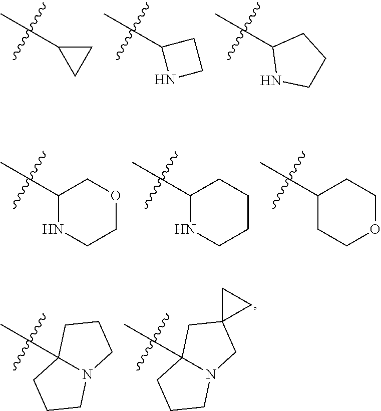

each of which is optionally substituted with one or more groups independently selected from: deuterium, halogen, —CN, oxo, C₁₋₆ alkyl, C₁₋₆ alkylidene, C₁₋₆ haloalkyl, and 3-8 membered heterocyclyl; wherein the C₁₋₆ alkyl, C₁₋₆ alkylidene and 3-8 membered heterocyclyl are each optionally substituted with one or more R₁₇; more preferably, R₁₂ is selected from

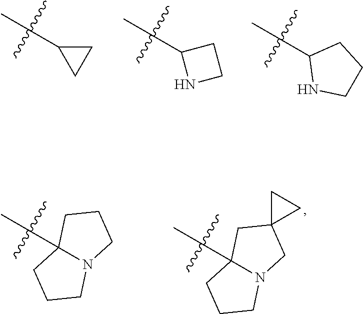

each of which is optionally substituted with one or more groups independently selected from: deuterium, halogen, —CN, oxo, C₁₋₆ alkyl, C₁₋₆ alkylidene, C₁₋₆ haloalkyl, and 3-8 membered heterocyclyl; wherein the C₁₋₆ alkyl, C₁₋₆ alkylidene and 3-8 membered heterocyclyl are each optionally substituted with one or more R₁₇; further preferably, R₁₂ is

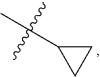 [SMILES: *C1CC1]

which is optionally substituted with one or more groups independently selected from: —CN and C₁₋₆ alkyl; wherein the C₁₋₆ alkyl is optionally substituted with one or more R₁₇; or R₁₂ is

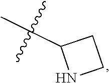 [SMILES: *C1CCN1]

which is optionally substituted with one or more groups independently selected from: C₁₋₆ haloalkyl; or R₁₂ is

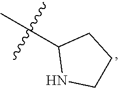 [SMILES: *C1CCCN1 ; ","]

which is optionally substituted with one or more groups independently selected from: deuterium, halogen, C₁₋₆ alkyl, C₁₋₆ haloalkyl, and 3-8 membered heterocyclyl; wherein the C₁₋₆ alkyl and 3-8 membered heterocyclyl are each optionally substituted with one or more R₁₇; or R₁₂ is

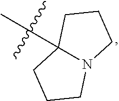 [SMILES: *C12CCCN1CCC2]

which is optionally substituted with one or more groups independently selected from: deuterium, halogen, and C₁₋₆ alkylidene; wherein the C₁₋₆ alkylidene is optionally substituted with one or more R₁₇;
R₁₇ is selected from deuterium, halogen, —OH, 3-8 membered heterocyclyl, and —NRₐR_{b}, wherein the 3-8 membered heterocyclyl is optionally substituted with one or more groups independently selected from: halogen, C₁₋₆ alkyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —CO(C₁₋₆ alkyl), and —S(O)₂(C₁₋₆ alkyl); preferably, R₁₇ is selected from deuterium, halogen, —OH, 3-8 membered heterocyclyl, —N(C₁₋₆ alkyl)₂, and —N(C₁₋₆ alkyl)(C₃₋₈ cycloalkyl), wherein the C₁₋₆ alkyl is optionally substituted with one or more —OH, and the 3-8 membered heterocyclyl is optionally substituted with one or more groups independently selected from: C₃₋₈ cycloalkyl, 3-8 membered heterocyclyl, and —(C₁₋₆ alkyl)-OH; and more preferably, R₁₇ is selected from deuterium, and halogen, for example, F;
R₄ is selected from C₁₋₆ alkyl, C₃₋₈ cycloalkyl, and 4-8 membered heterocyclyl, each of which is optionally substituted with one or more R₁₃; preferably, R₄ is selected from C₁₋₆ alkyl, and C₃₋₈ cycloalkyl, each of which is optionally substituted with one or more R₁₃; more preferably, R₄ is C₁₋₆ alkyl, which is optionally substituted with one or more R₁₃; and further preferably, R₄ is C₁₋₆ alkyl, which is substituted with one or more R₁₃;
R₁₃ is selected from deuterium and 5-10 membered heteroaryl; wherein the 5-10 membered heteroaryl is optionally substituted with one or more R₁₄; preferably, R₁₃ is selected from

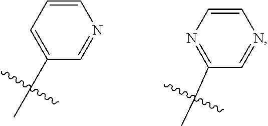 [SMILES: *c1cccnc1 ; *Cc1cnccn1]

each of which is optionally substituted with one or more R₁₄; more preferably, R₁₃ is

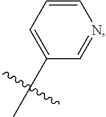 [SMILES: *c1cccnc1]

which is optionally substituted with one or more R₁₄; or R₁₃ is

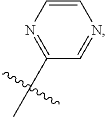 [SMILES: *c1cnccn1]

which is optionally substituted with one or more R₁₄;
R₁₄ is selected from halogen and —NRₐR_{b}; and preferably, R₁₄ is —NH₂;
R₅, R₆, R₇ and R₈ are each independently selected from hydrogen, deuterium, C₁₋₆ alkyl, C₁₋₆ haloalkyl, and C₃₋₈ cycloalkyl; or R₅ and R₆, R₇ and R₈, or R₅ and R₇ together with the carbon atoms to which they are attached form C₃₋₆ cycloalkyl or 3-6 membered heterocyclyl; preferably, R₅, R₆, R₇ and R₈ are each independently selected from hydrogen, deuterium and C₁₋₃ alkyl; or R₅ and R₆ together with the carbon atom to which they are attached form cyclopropyl; or R₅ and R₇ together with the carbon atoms to which they are attached form cyclopropyl, cyclopentyl, tetrahydrofuranyl, or tetrahydropyranyl; more preferably, R₅, R₆, R₇ and R₈ are all hydrogen; or R₅ is C₁₋₃ alkyl, and R₆, R₇ and R₈ are each independently selected from hydrogen and deuterium; or R₅ and R₇ together with the carbon atoms to which they are attached form cyclopentyl, tetrahydrofuranyl, or tetrahydropyranyl; and
Rₐ, R_{b} and R_{d} are each independently selected from hydrogen, C₁₋₆ alkyl, and C₃₋₈ cycloalkyl; wherein the C₁₋₆ alkyl and C₃₋₈ cycloalkyl are each optionally substituted with one or more groups independently selected from: —OH, —NR_{g}Rₕ, —C(O)NR_{g}Rₕ, and —NHC(O)Rᵢ; wherein R_{g}, Rₕ and Rᵢ are each independently selected from hydrogen, C₁₋₆ alkyl, and —(C₁₋₆ alkyl)-NH₂.

Embodiment 25. The compound or the pharmaceutically acceptable salt thereof, or the solvate, the racemic mixture, the enantiomer, the diastereomer or the tautomer thereof according to embodiment 24, wherein R₄ is C₁₋₆ alkyl substituted with R₁₃;
R₁₃ is selected from

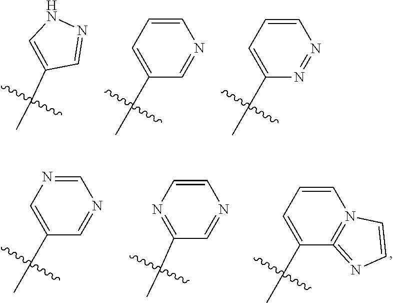

each of which is optionally substituted with one or more R₁₄;
preferably, R₁₃ is selected from

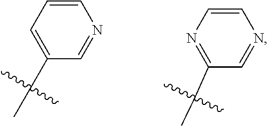 [SMILES: *c1cccnc1 ; *Cc1cnccn1]

each of which is optionally substituted with one or more R₁₄;
more preferably, R₁₃ is

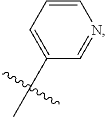 [SMILES: *c1cccnc1]

which is optionally substituted with one or more R₁₄; or R₁₃ is

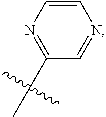 [SMILES: *c1cnccn1]

which is optionally substituted with one or more R₁₄; and
R₁₄ is independently selected from halogen and —NH₂.

Embodiment 26. A compound or the pharmaceutically acceptable salt thereof, or the solvate, the racemic mixture, the enantiomer, the diastereomer or the tautomer thereof, which is selected from:

| No. | Structural formula |
|---|---|
| 1 | 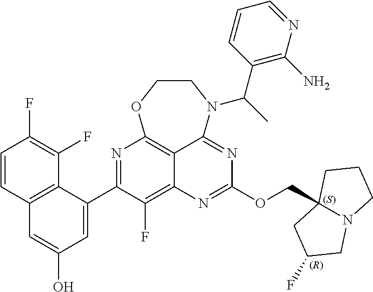 |
| 2 | 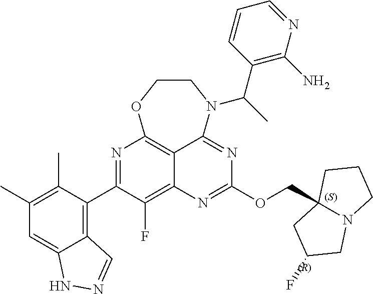 |
| 3 | 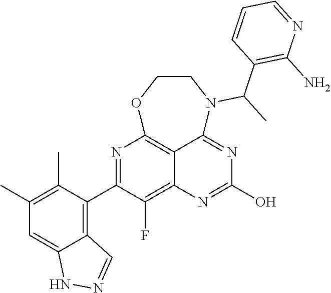 [SMILES: Cc1cc2[nH]ncc2c(-c2nc3c4c(nc(O)nc4c2F)N(C(C)c2cccnc2N)CCO3)c1C] |
| 4 | 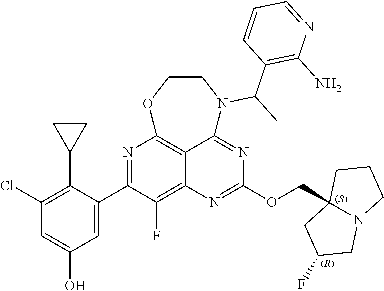 |
| 5 | 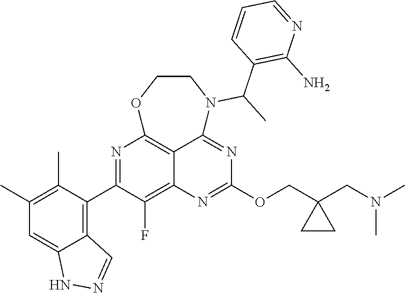 |
| 6 | 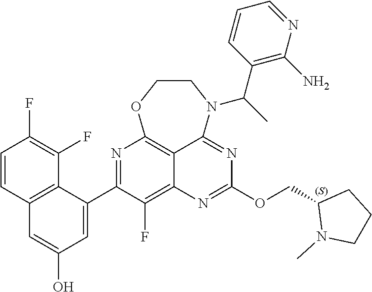 |
| 7 | 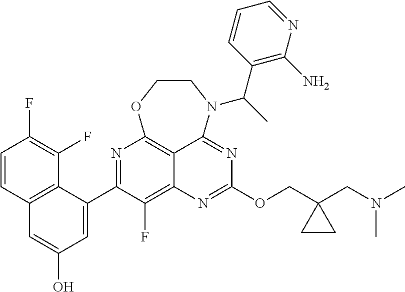 [SMILES: CC(c1cccnc1N)N1CCOc2nc(-c3cc(O)cc4ccc(F)c(F)c34)c(F)c3nc(OCC4(CN(C)C)CC4)nc1c23] |
| 8 | 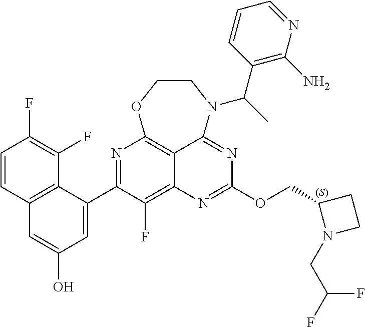 |
| 9 | 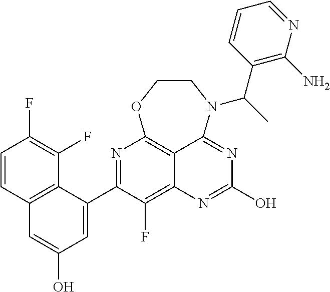 [SMILES: CC(c1cccnc1N)N1CCOc2nc(-c3cc(O)cc4ccc(F)c(F)c34)c(F)c3nc(O)nc1c23] |
| 10 | 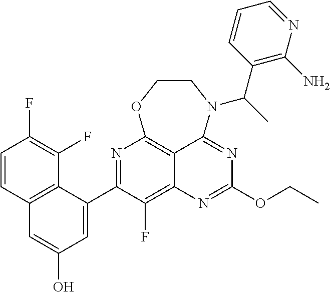 [SMILES: CCOc1nc2c3c(nc(-c4cc(O)cc5ccc(F)c(F)c45)c(F)c3n1)OCCN2C(C)c1cccnc1N] |
| 11 | 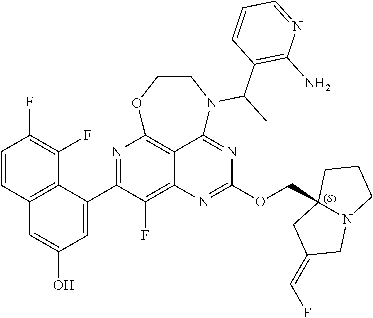 |
| 12 | 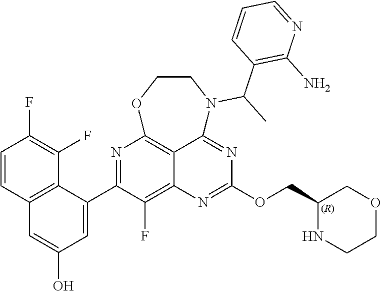 |
| 13 | 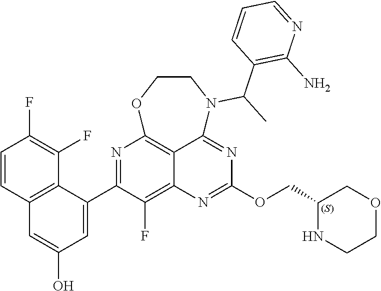 |
| 14 | 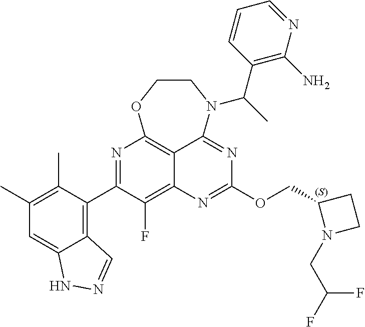 |
| 15 | 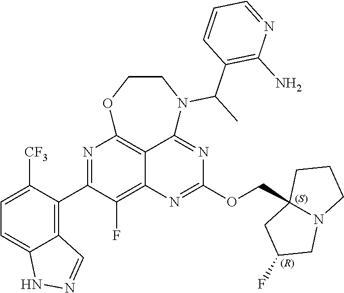 |
| 16 | 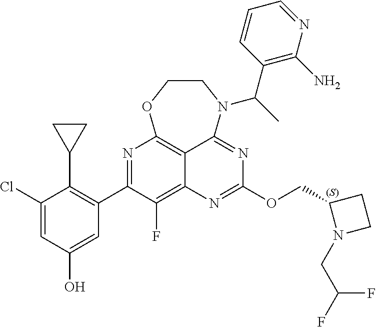 |
| 17 | 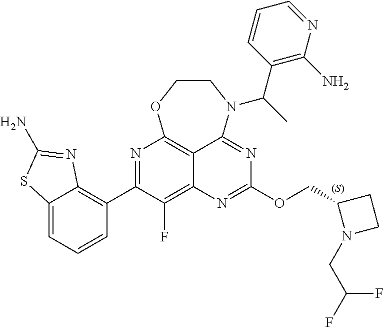 |
| 18 | 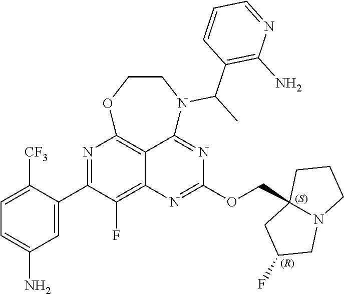 |
| 19 | 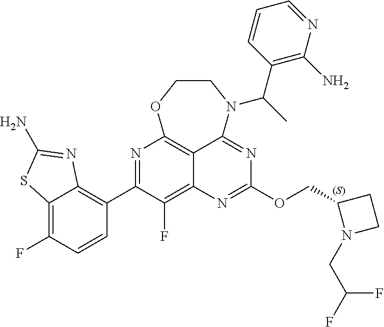 |
| 22 | 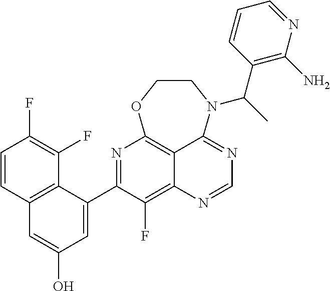 [SMILES: CC(c1cccnc1N)N1CCOc2nc(-c3cc(O)cc4ccc(F)c(F)c34)c(F)c3ncnc1c23] |
| 20 and 21 | 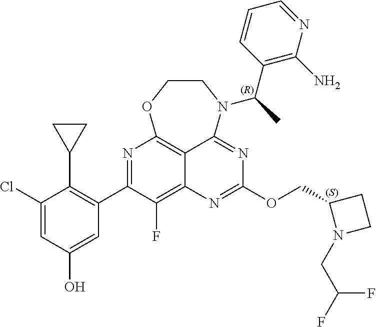 + 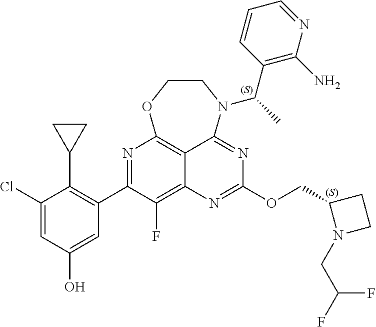 20 & 21 |
| 23 and 24 | 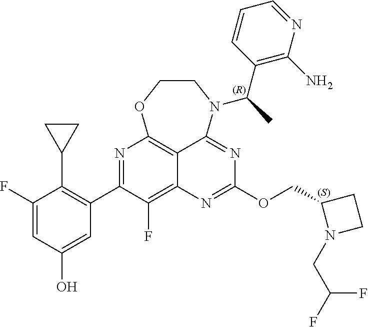 + 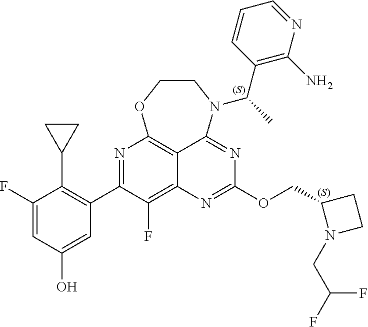 23 & 24 |
| 25 | 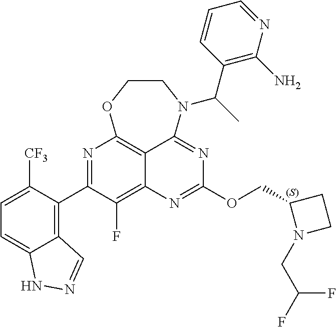 |
| 28 | 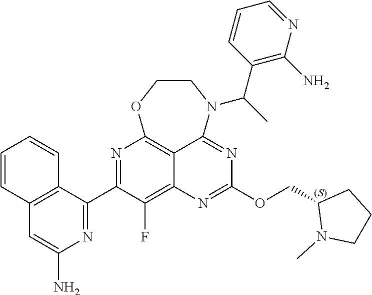 |
| 26 and 27 | 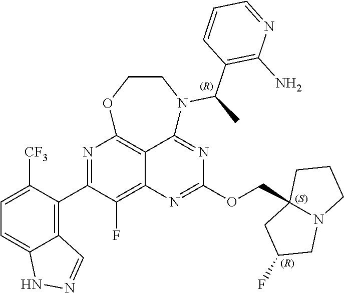 + 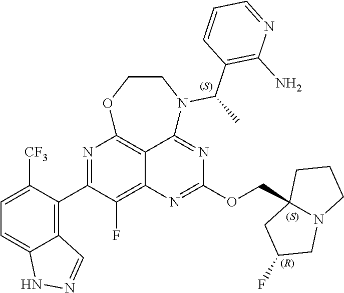 26 & 27 |
| 29 and 30 | 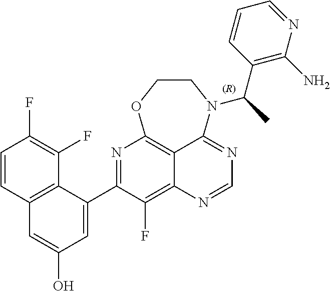 + 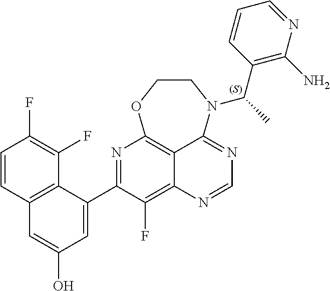 29 & 30 |
| 31 and 32 | 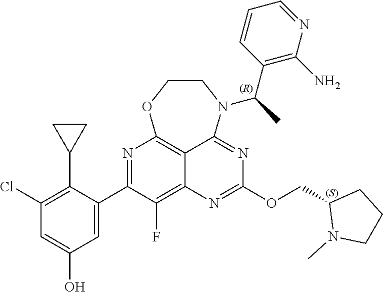 + 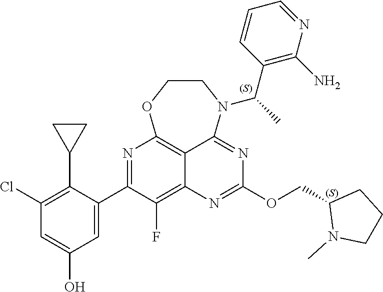 31 & 32 |
| 34 and 35 | 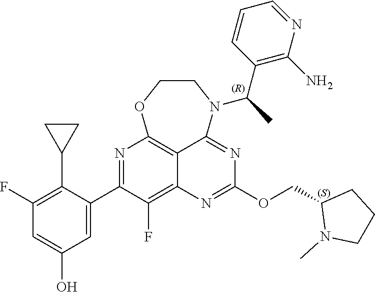 + 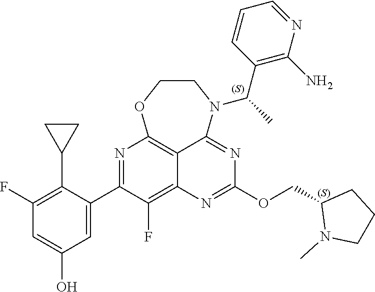 34 & 35 |
| 33 | 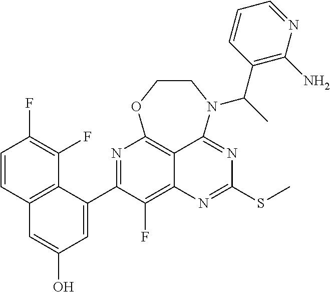 [SMILES: CSc1nc2c3c(nc(-c4cc(O)cc5ccc(F)c(F)c45)c(F)c3n1)OCCN2C(C)c1cccnc1N] |
| 36 | 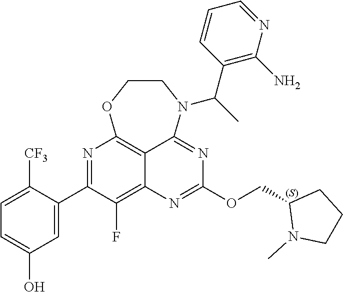 |
| 37 | 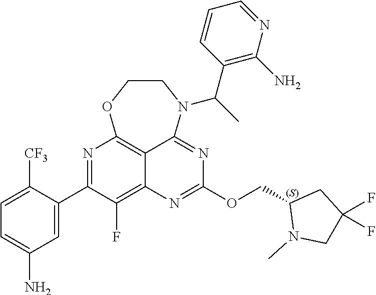 |
| 40 | 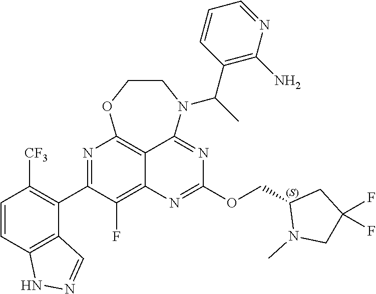 |
| 38 and 39 | 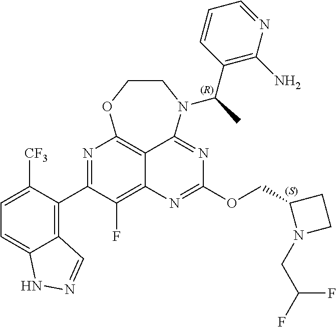 + 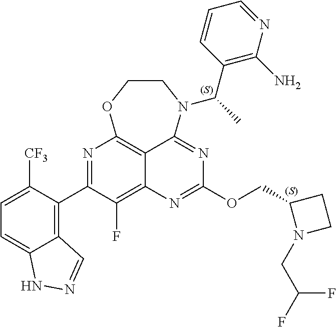 38 & 39 |
| 41 and 42 | 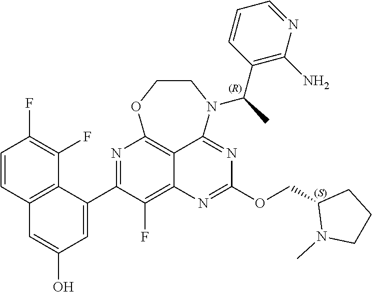 + 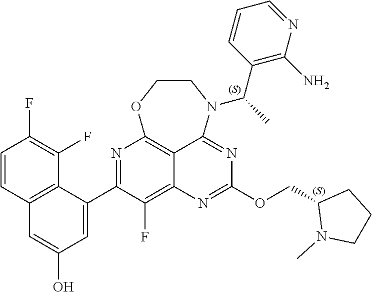 41 & 42 |
| 43 and 44 | 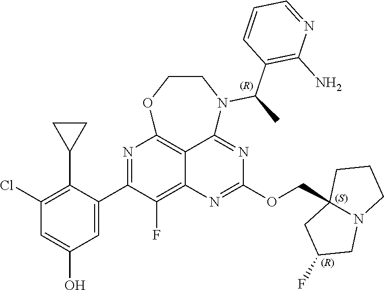 + 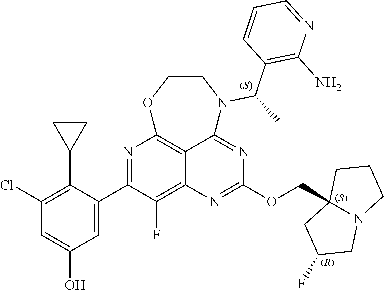 43 & 44 |
| 48 and 49 | 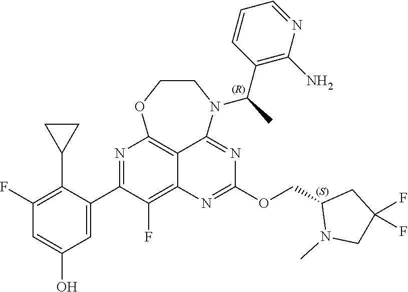 + 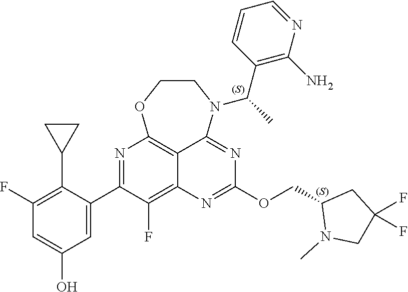 48 & 49 |
| 45 | 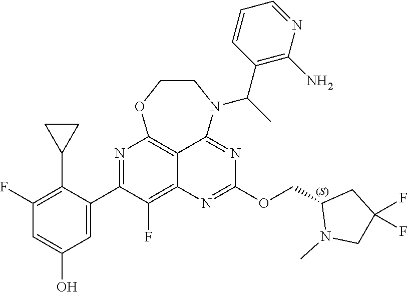 |
| 46 | 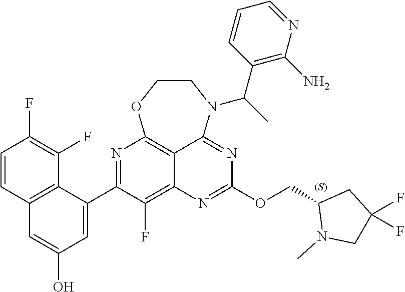 |
| 47 | 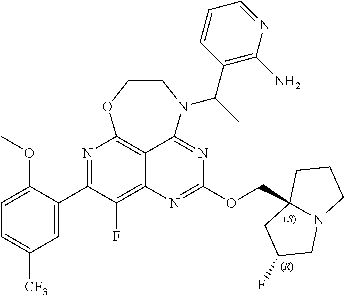 |
| 59 | 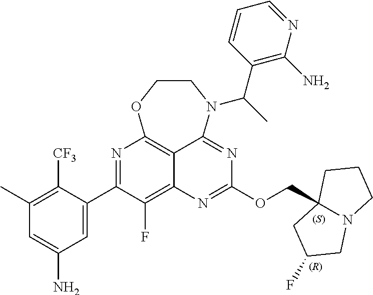 |
| 50 and 51 | 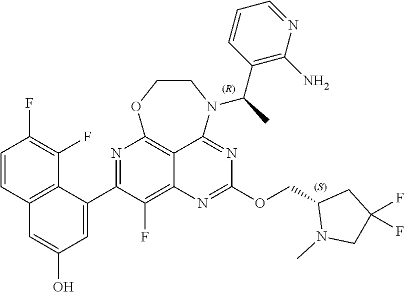 + 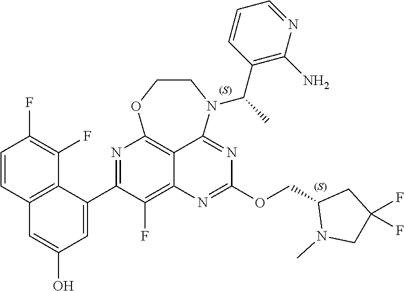 50 & 51 |
| 53 and 54 | 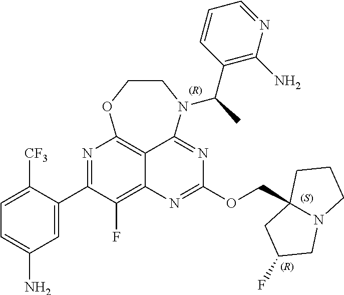 + 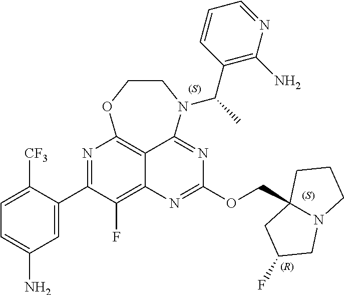 53 & 54 |
| 55 and 56 | 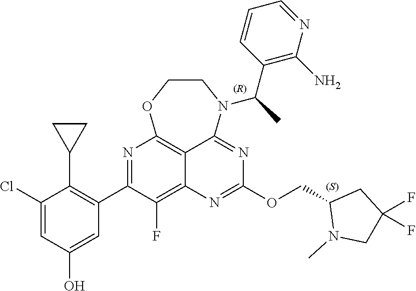 + 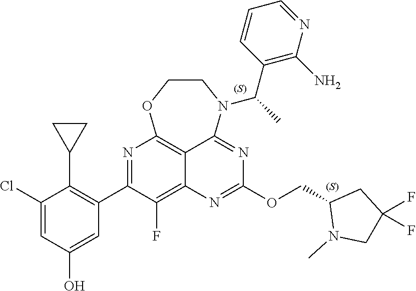 55 & 56 |
| 57 and 58 | 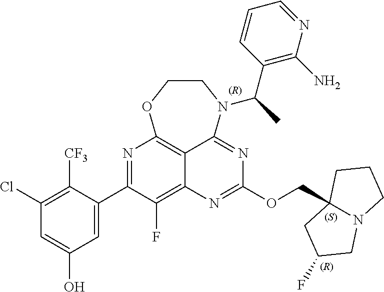 + 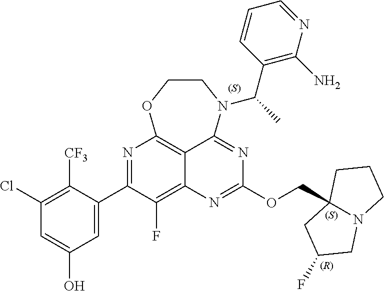 57 & 58 |
| 60 | 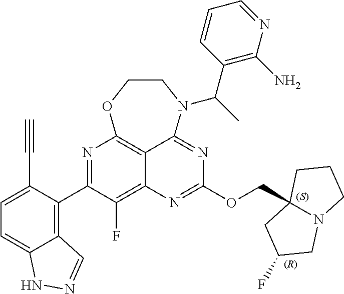 |
| 63 | 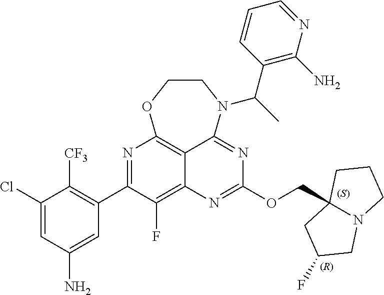 |
| 61 and 62 | 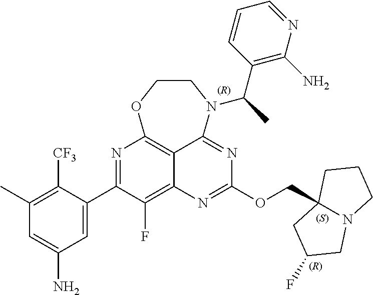 + 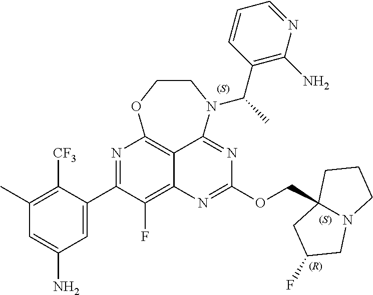 61 & 62 |
| 71 and 72 | 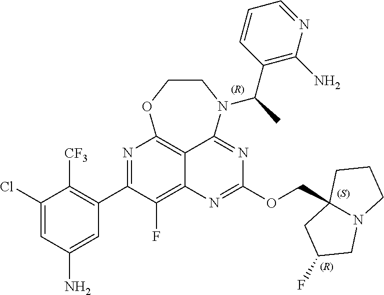 + 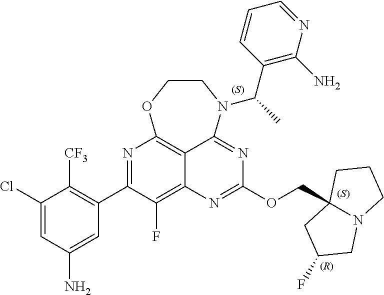 71 & 72 |
| 64 | 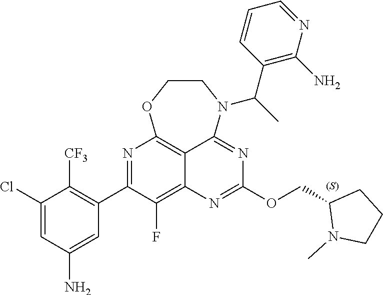 |
| 65 | 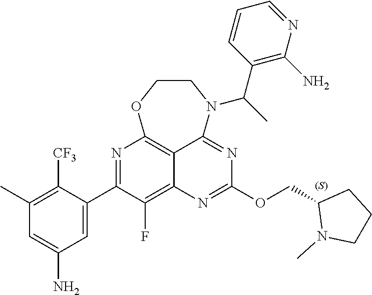 |
| 66 | 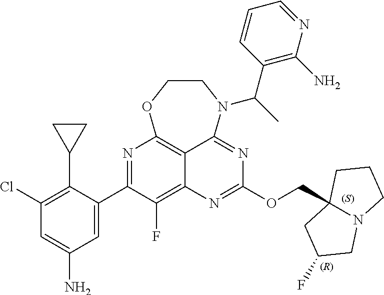 |
| 67 | 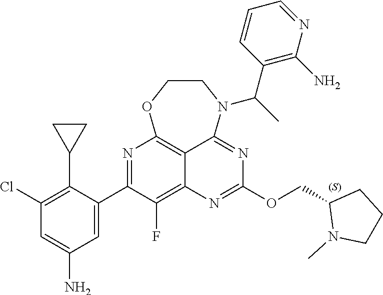 |
| 68 | 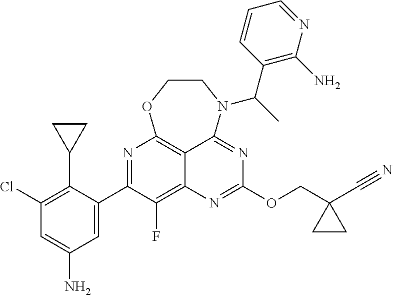 [SMILES: CC(c1cccnc1N)N1CCOc2nc(-c3cc(N)cc(Cl)c3C3CC3)c(F)c3nc(OCC4(C#N)CC4)nc1c23] |
| 69 | 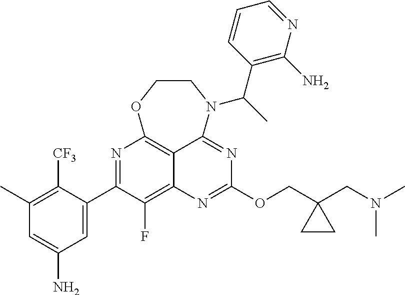 |
| 70 | 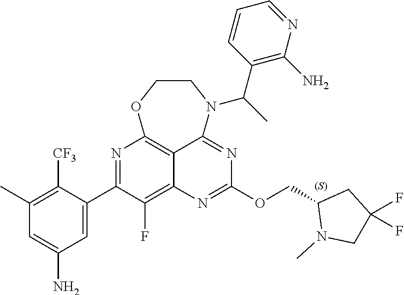 |
| 73 | 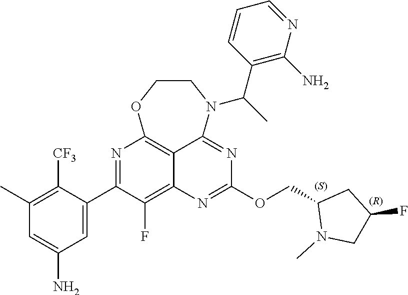 |
| 74 and 75 | 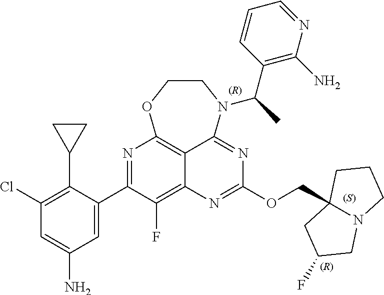 + 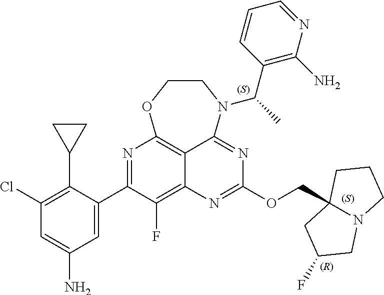 74 & 75 |
| 76 and 77 | 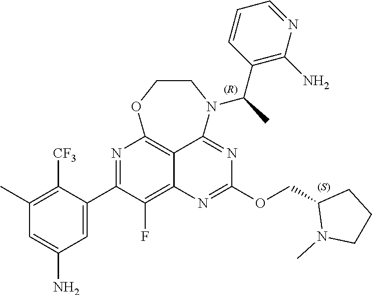 + 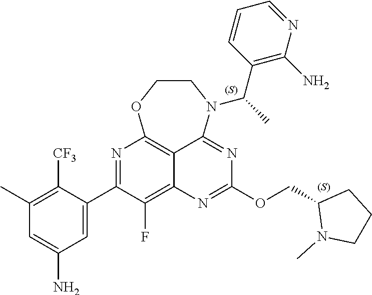 76 & 77 |
| 78 and 79 | 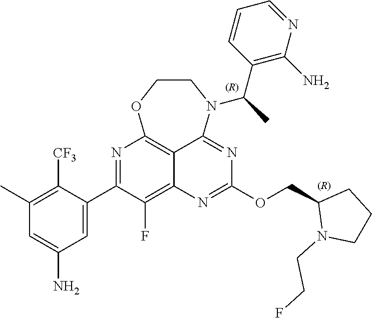 + 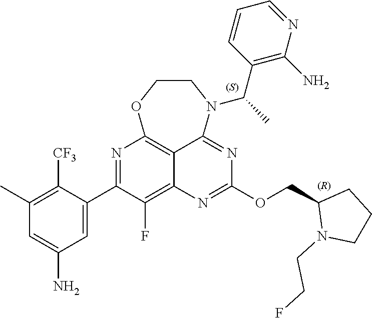 78 & 79 |
| 80 and 81 | 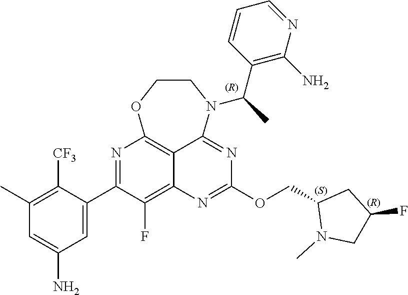 + 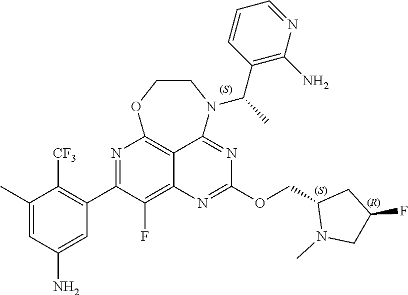 80 & 81 |
| 82 | 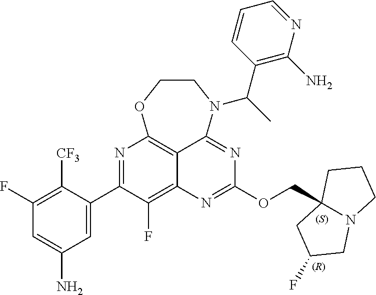 |
| 83 | 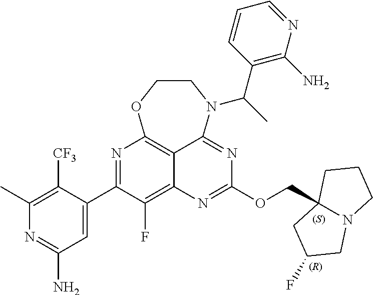 |
| 85 and 86 | 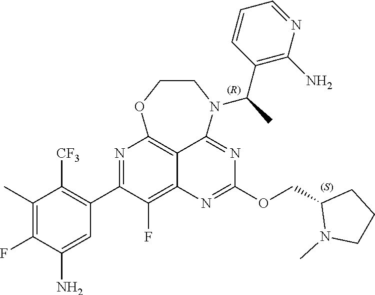 + 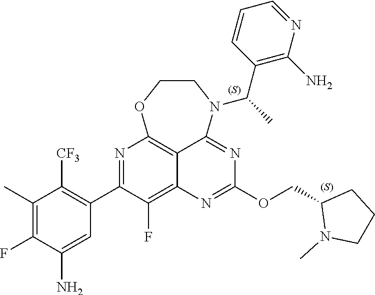 85 & 86 |
| 88 and 89 | 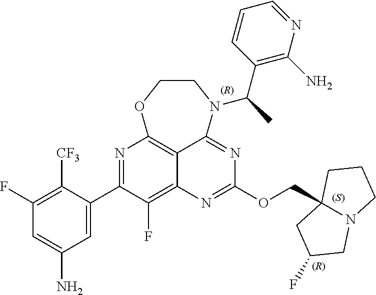 + 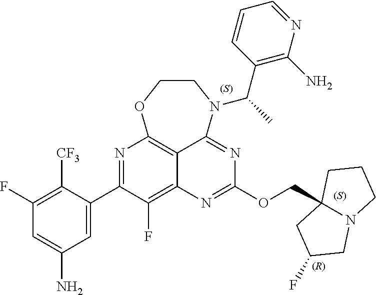 88 & 89 |
| 87 | 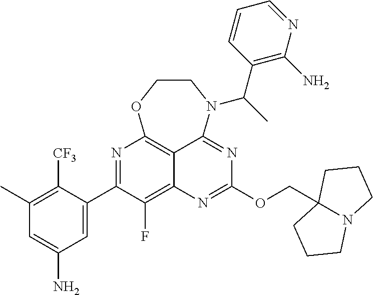 [SMILES: Cc1cc(N)cc(-c2nc3c4c(nc(OCC56CCCN5CCC6)nc4c2F)N(C(C)c2cccnc2N)CCO3)c1C(F)(F)F] |
| 94 | 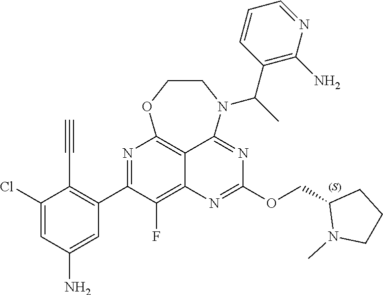 |
| 90 and 91 | 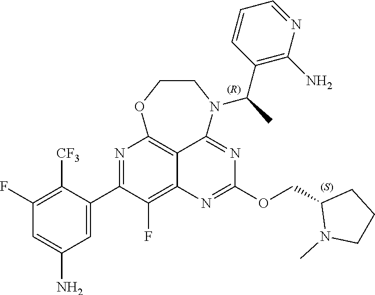 + 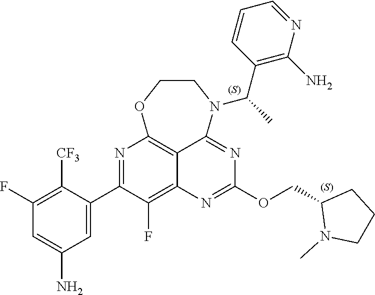 90 & 91 |
| 92 and 93 | 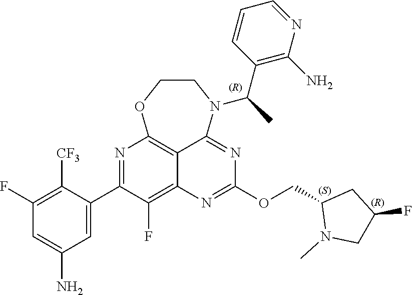 + 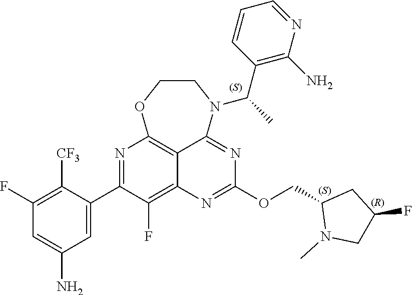 92 & 93 |
| 97 | 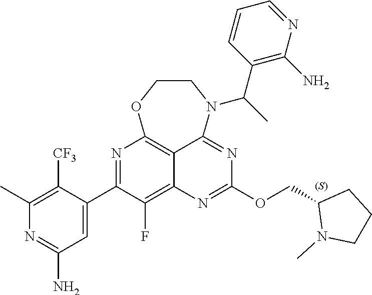 |
| 104 | 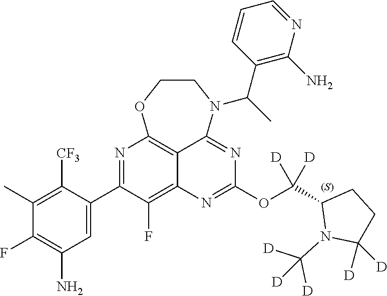 |
| 95 and 96 | 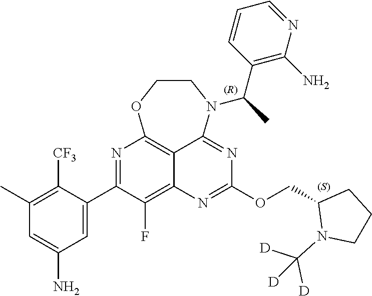 + 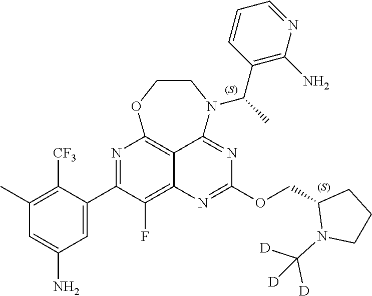 95 & 96 |
| 98 and 99 | 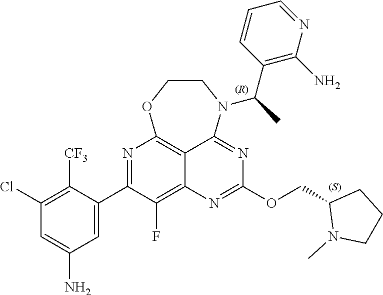 + 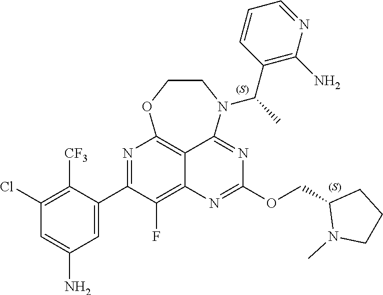 98 & 99 |
| 100 and 101 | 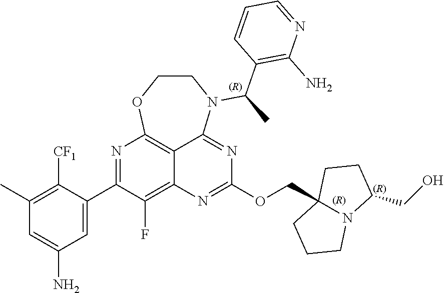 + 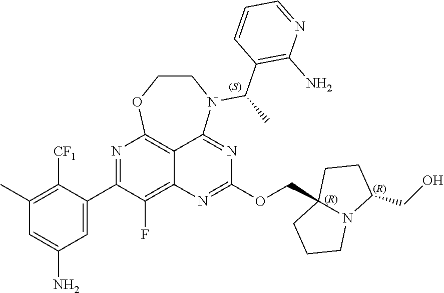 100 & 101 |
| 102 and 103 | 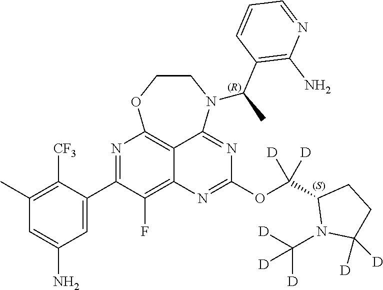 + 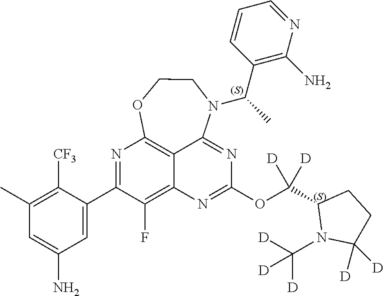 102 & 103 |
| 105 | 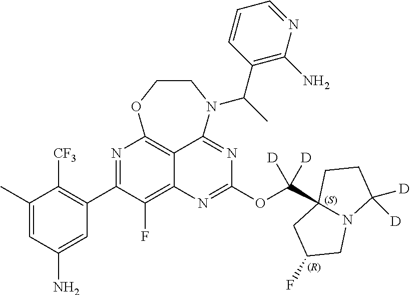 |
| 108 | 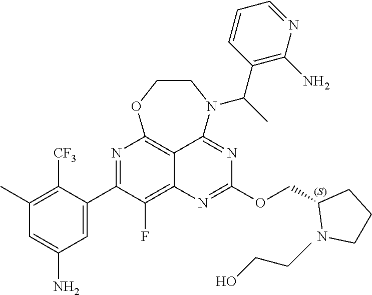 |
| 106 and 107 | 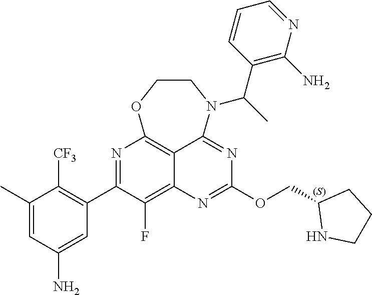 + 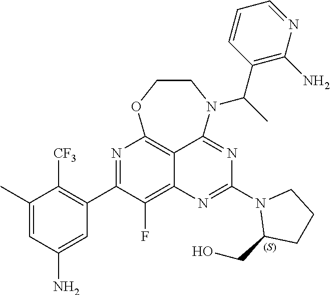 |
| 109 and 110 | 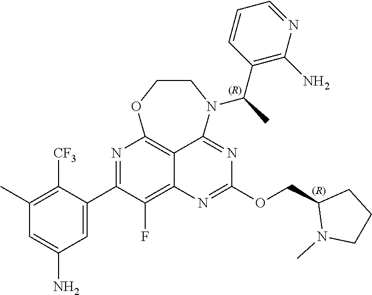 + 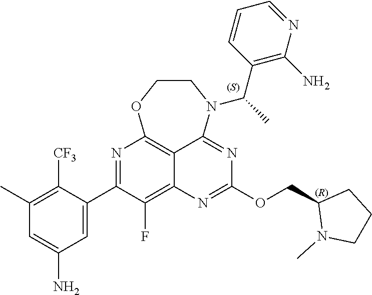 109 & 110 |
| 111 | 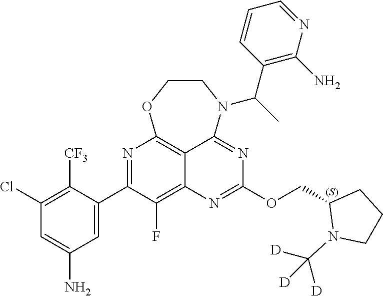 |
| 112 | 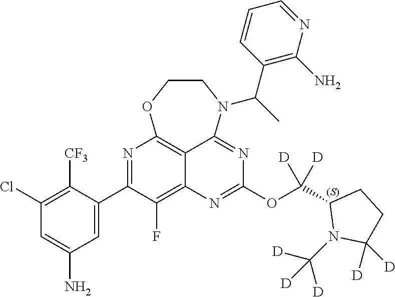 |
| 113 | 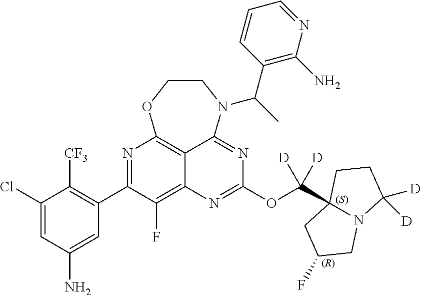 |
| 114 | 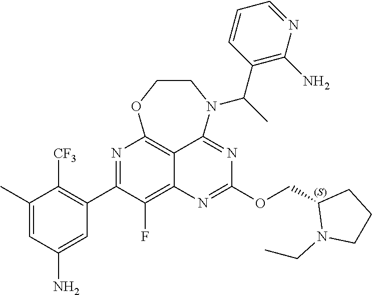 |
| 119 | 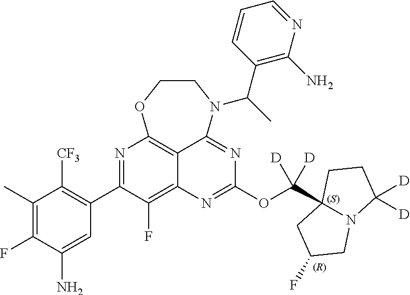 |
| 120 | 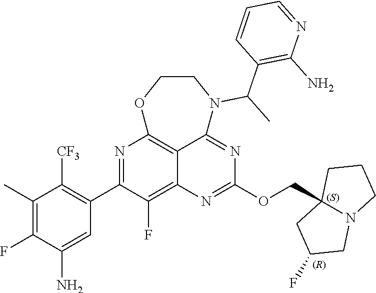 |
| 115 and 116 | 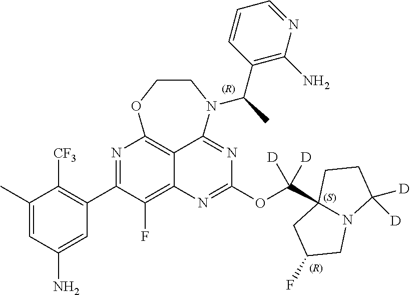 + 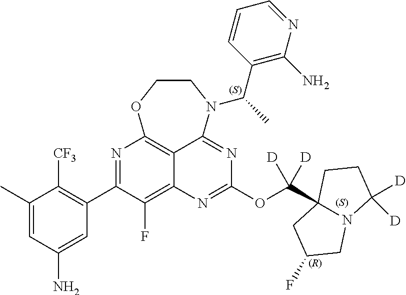 115 & 116 |
| 117 and 118 | 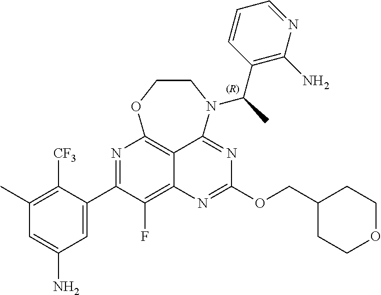 + 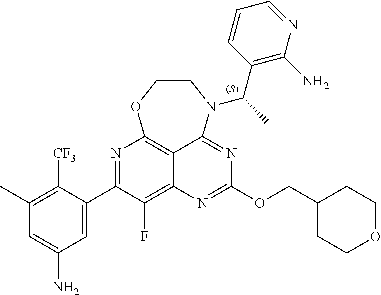 117 & 118 |
| 121 | 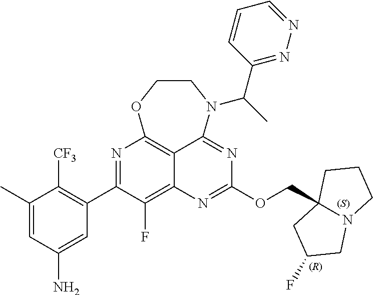 |
| 122 | 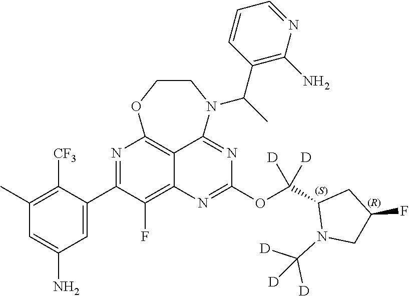 |
| 123 | 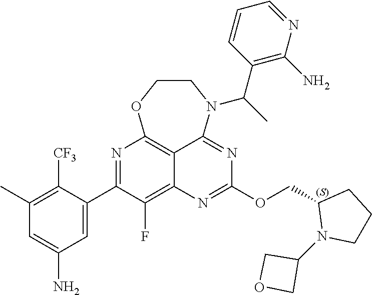 |
| 126 | 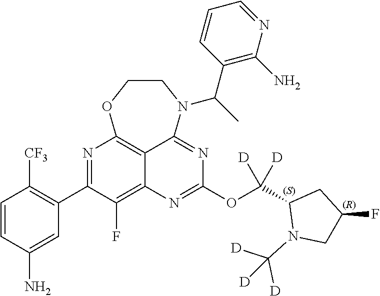 |
| 124 and 125 | 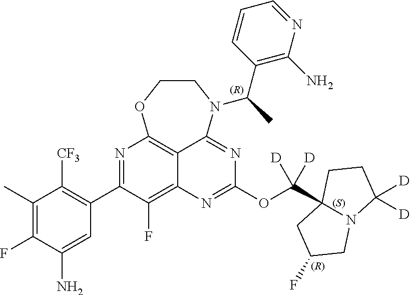 + 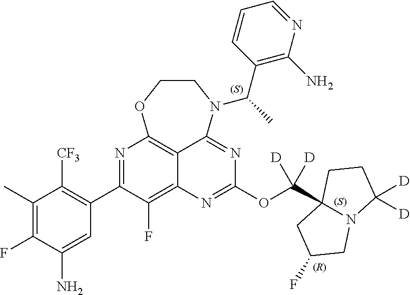 124 & 125 |
| 127 and 128 | 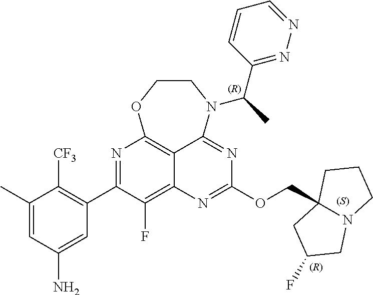 + 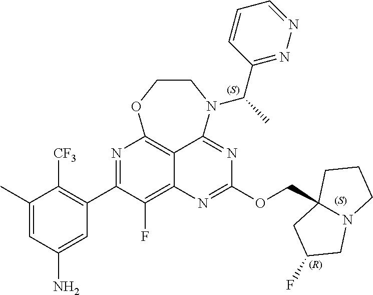 127 & 128 |
| 129 | 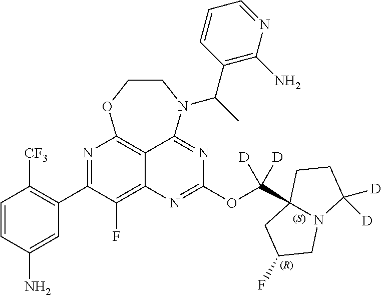 |
| 134 | 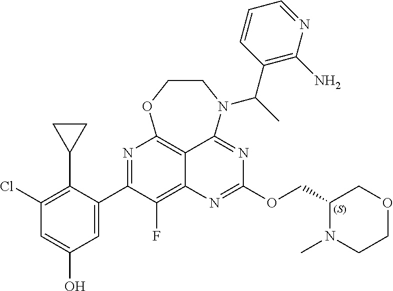 |
| 130 and 131 | 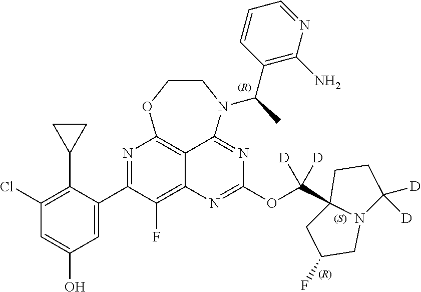 + 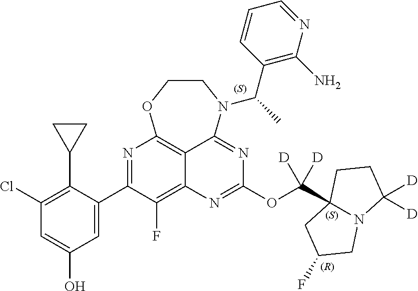 130 & 131 |
| 132 and 133 | 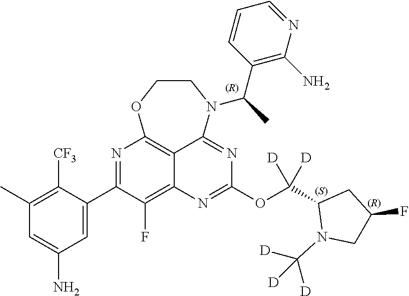 + 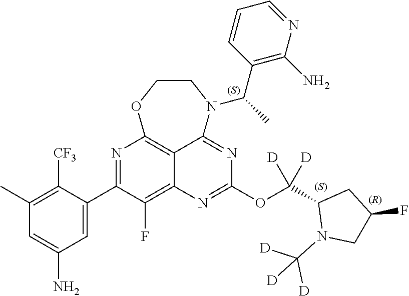 132 & 133 |
| 135 | 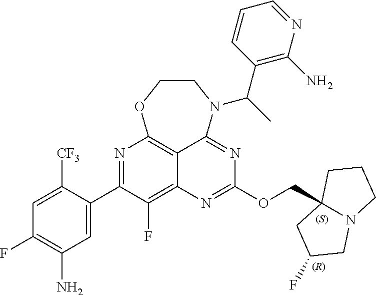 |
| 136 | 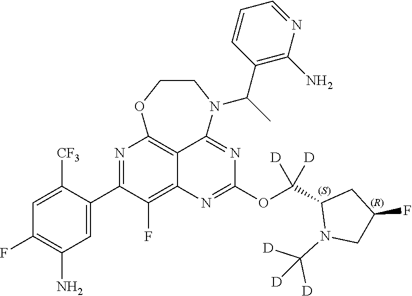 |
| 139 | 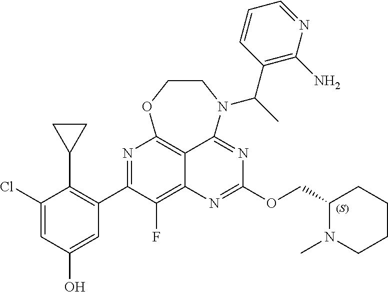 |
| 140 | 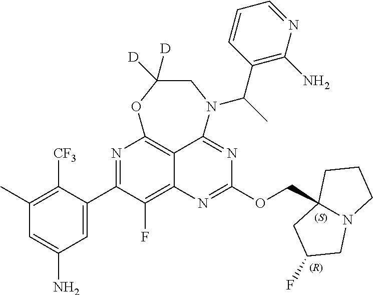 |
| 137 and 138 | 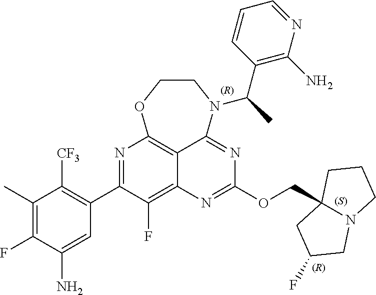 + 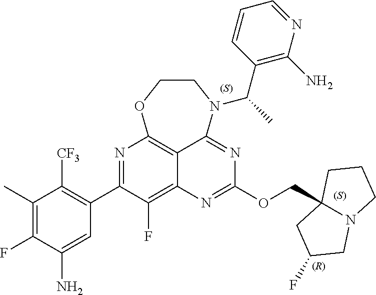 137 & 138 |
| 142 and 143 | 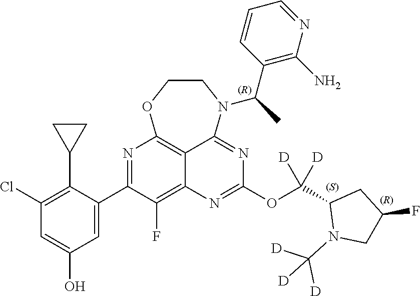 + 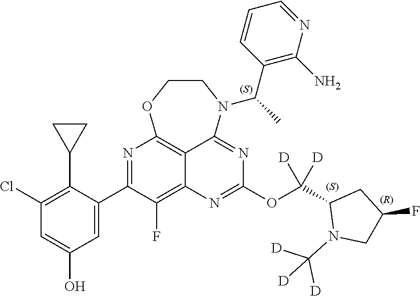 142 & 143 |
| 141 | 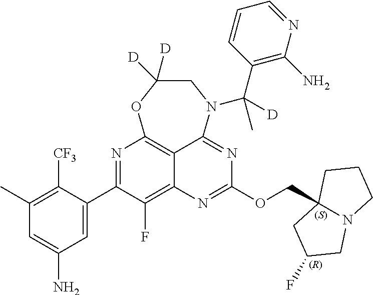 |
| 146 | 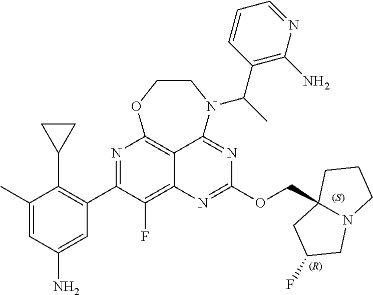 |
| 144 and 145 | 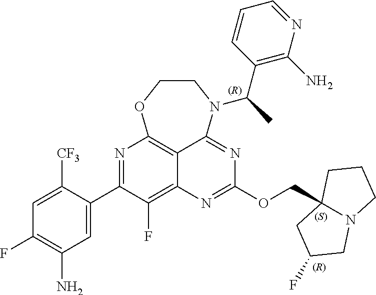 + 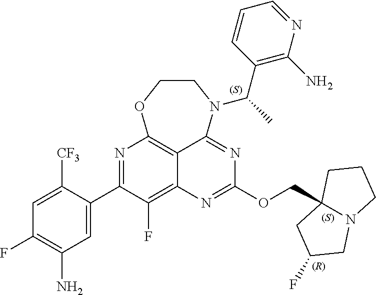 144 & 145 |
| 169 and 170 | 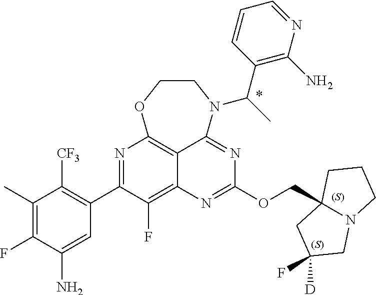 + 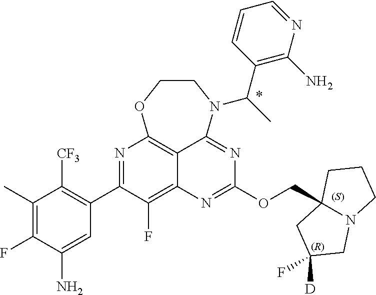 169 & 170 |
| 153 and 154 | 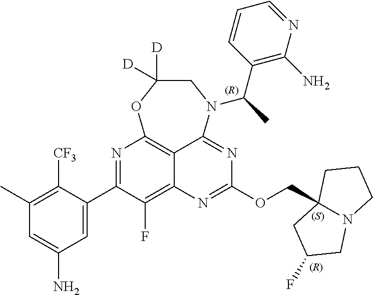 + 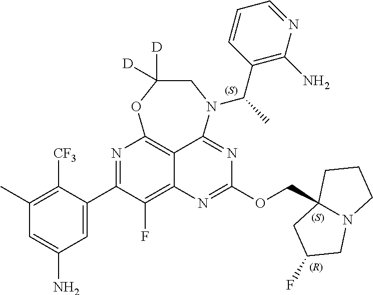 153 & 154 |
| 148 | 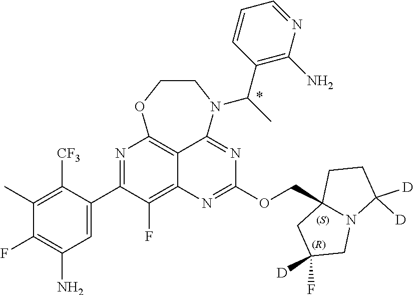 |
| 149 | 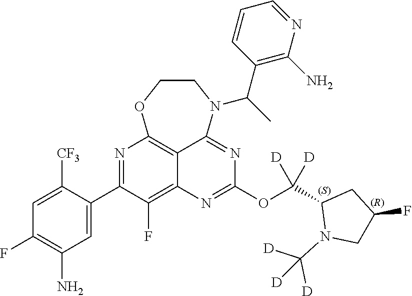 |
| 150 | 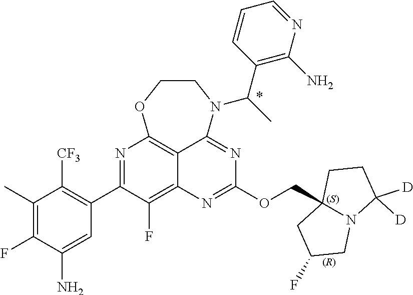 |
| 157 | 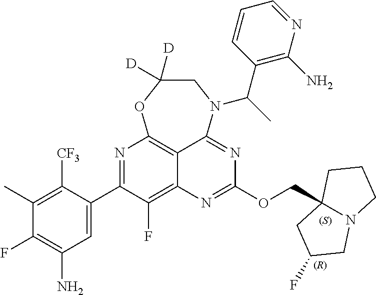 |
| 158 | 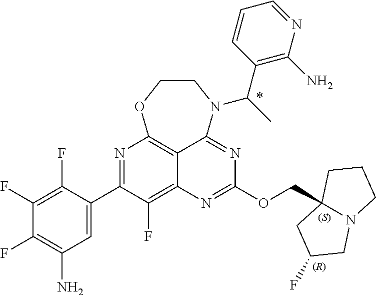 |
| 159 | 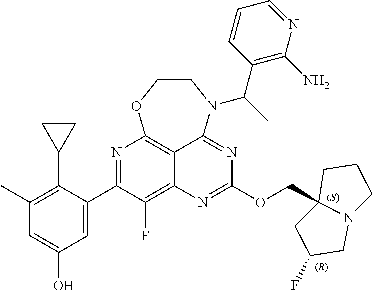 |
| 162 | 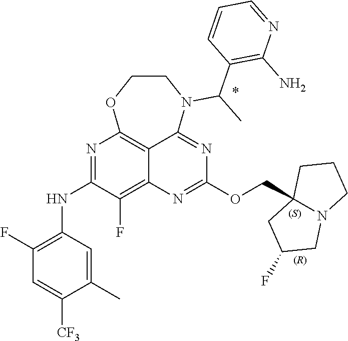 |
| 160 and 161 | 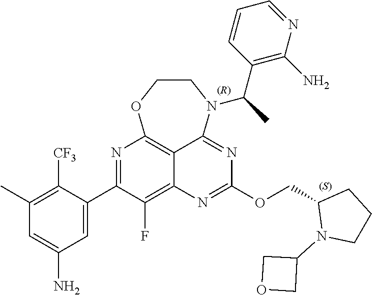 + 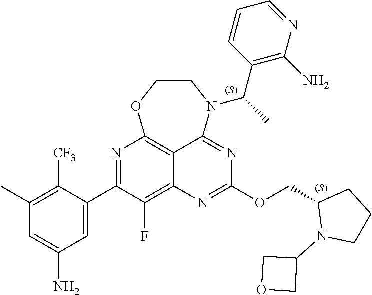 160 & 161 |
| 166 and 167 | 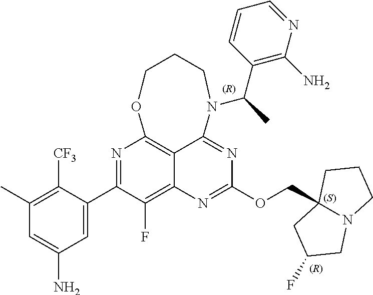 + 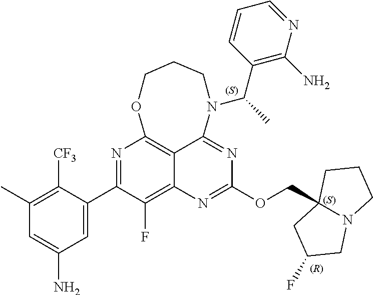 166 & 167 |
| 163 | 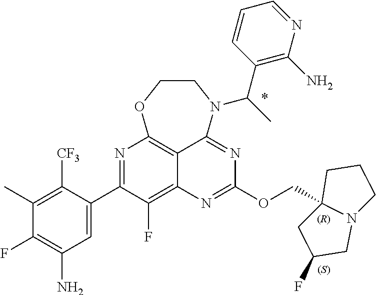 |
| 164 | 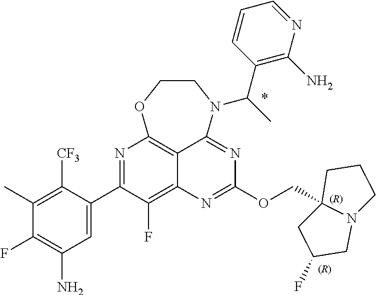 |
| 165 | 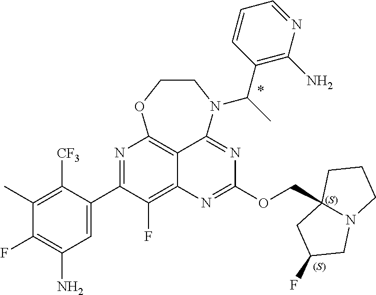 |
| 168 | 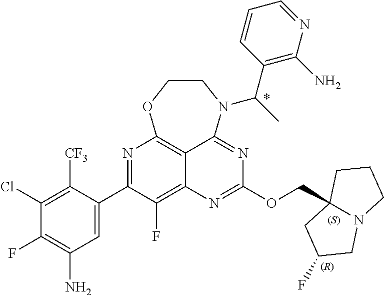 |
| 171 | 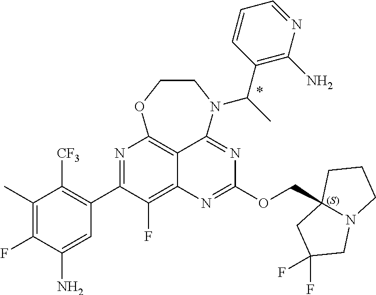 |
| 173 | 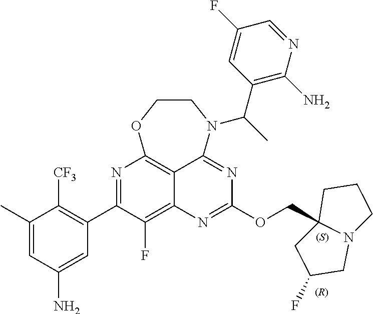 |
| 174 | 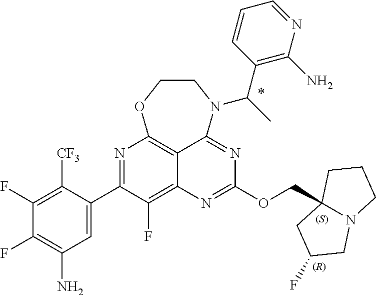 |
| 177 | 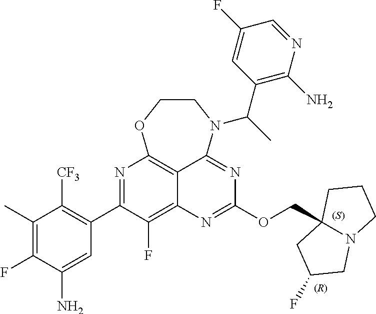 |
| 180 | 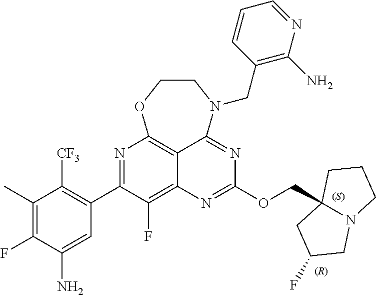 |
| 181 | 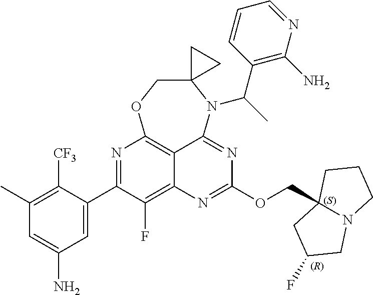 |
| 178 and 179 | 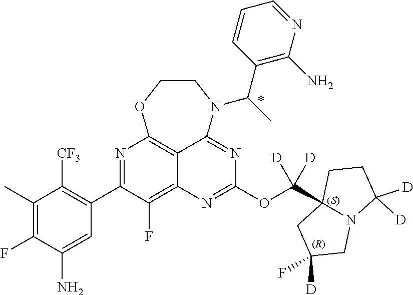 + 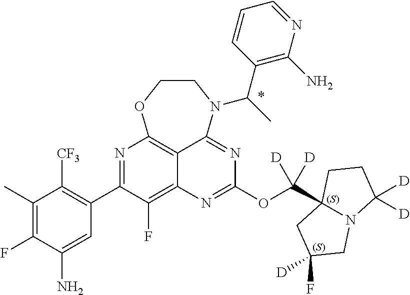 178 & 179 |
| 183 and 184 | 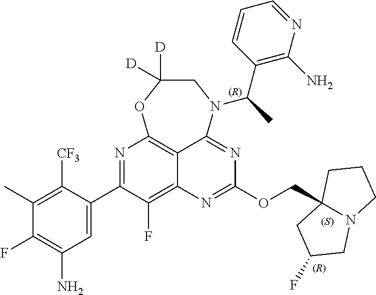 + 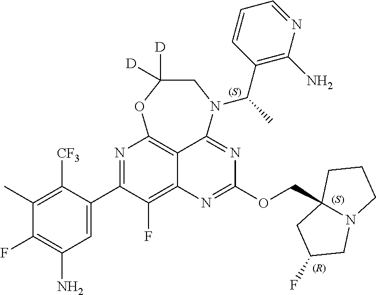 183 & 184 |
| 185 | 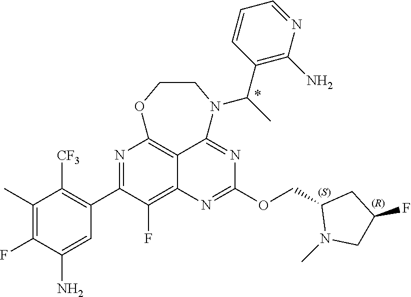 |
| 187 | 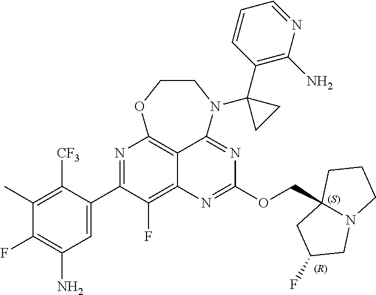 |
| 198 | 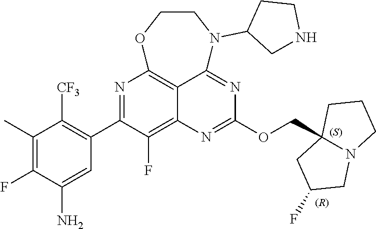 |
| 195 | 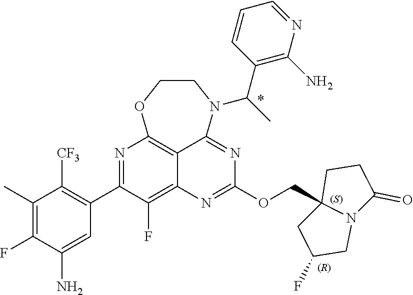 |
| 196 | 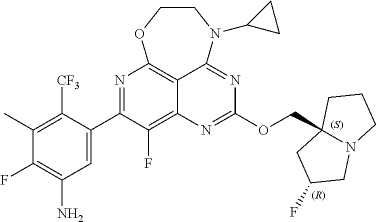 |
| 197 | 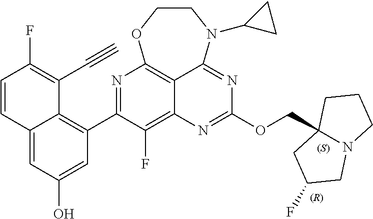 |
| 199 and 200 | 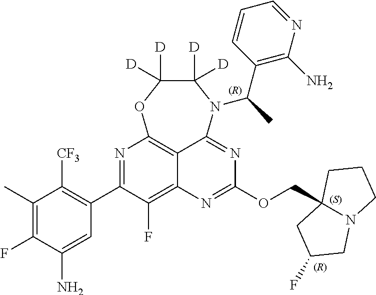 + 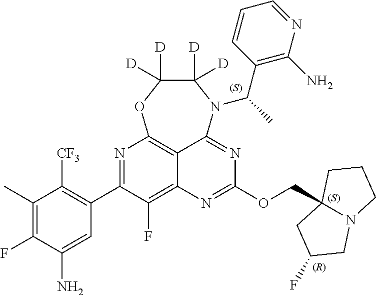 199 & 200 |
| 202 and 203 | 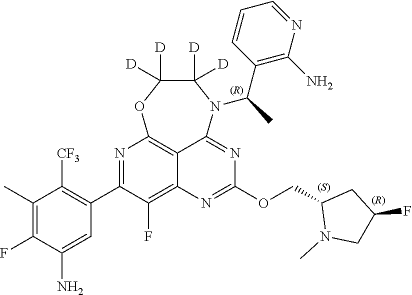 + 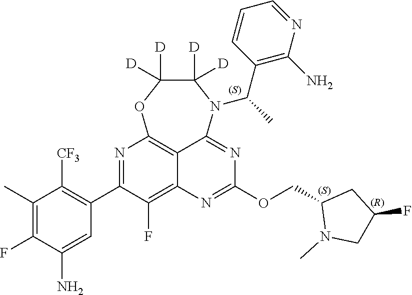 202 & 203 |
| 204 | 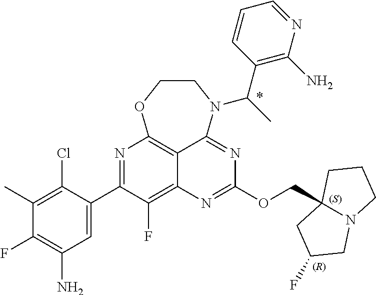 |
| 205 | 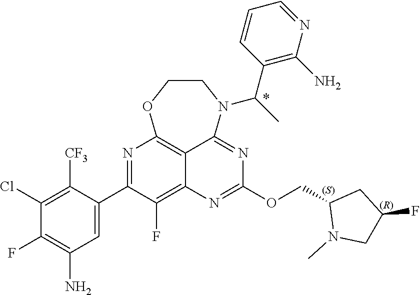 |
| 206 and 207 | 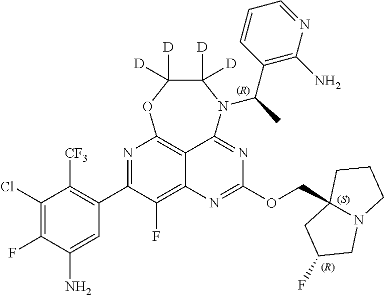 + 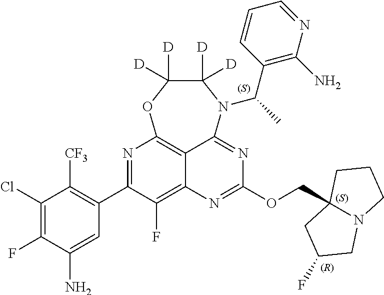 206 & 207 |
| 208 and 209 | 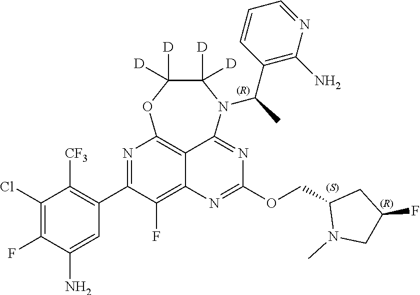 + 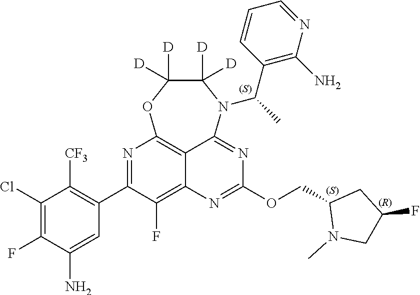 208 & 209 |
| 210 and 211 | 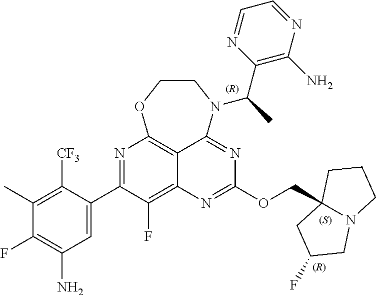 + 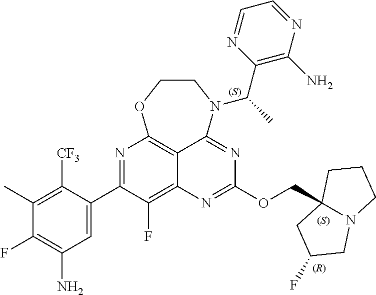 210 & 211 |
| 212 and 213 | 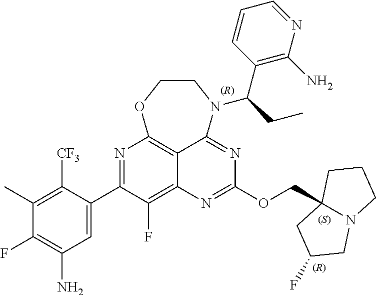 + 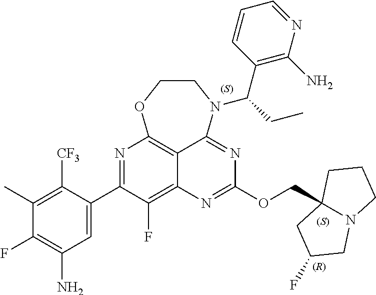 212 & 213 |
| 214 and 215 | 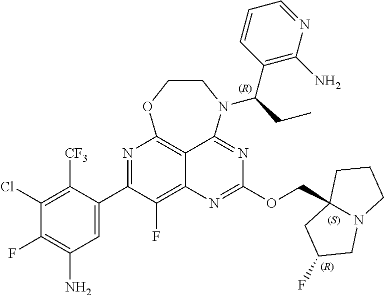 + 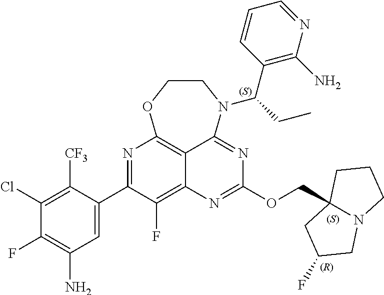 214 & 215 |
| 216 and 217 | 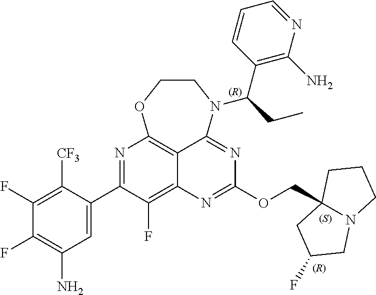 + 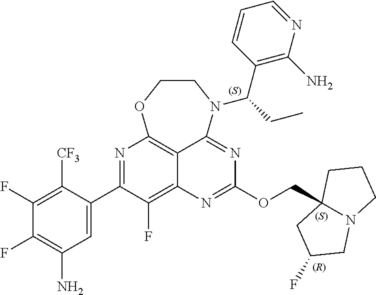 216 & 217 |
| 218 | 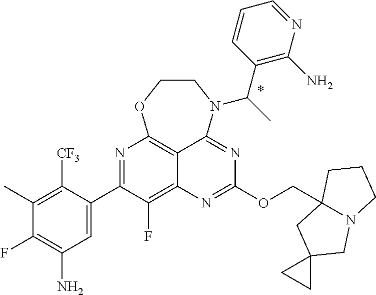 [SMILES: Cc1c(F)c(N)cc(-c2nc3c4c(nc(OCC56CCCN5CC5(CC5)C6)nc4c2F)N(C(C)c2cccnc2N)CCO3)c1C(F)(F)F] |
| 219 and 220 | 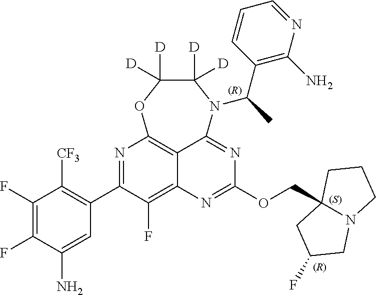 + 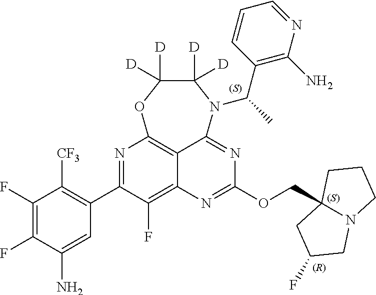 219 & 220 |
| 221 | 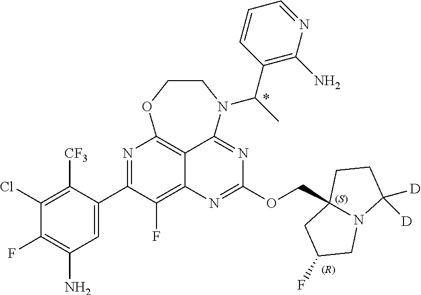 |
| 224 | 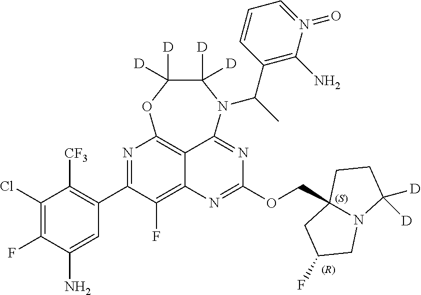 |
| 222 and 223 | 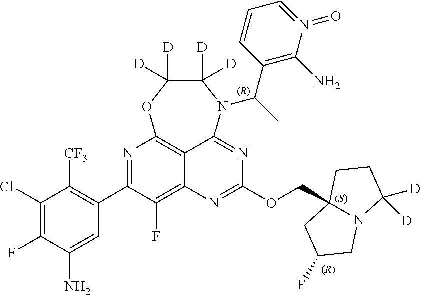 + 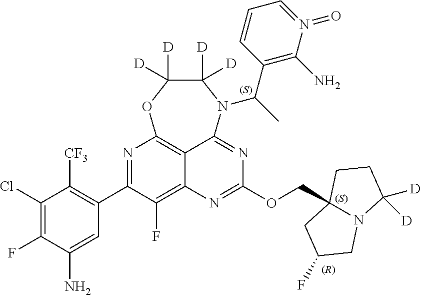 222 & 223 |
| 238 and 239 | 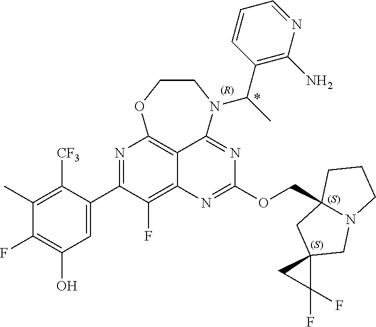 + 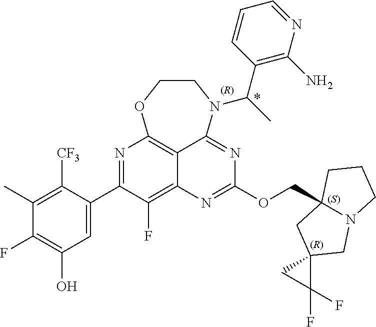 238 & 239 |
| 225 | 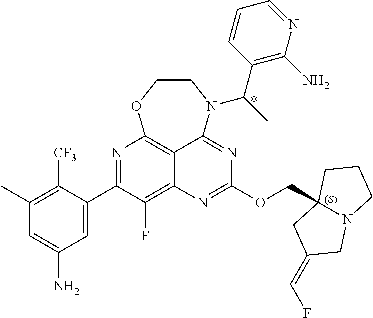 |
| 226 | 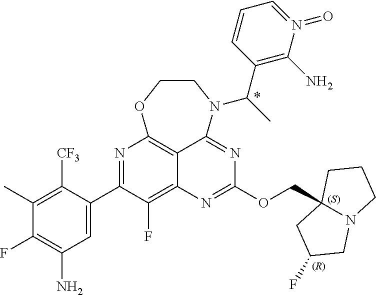 |
| 227 | 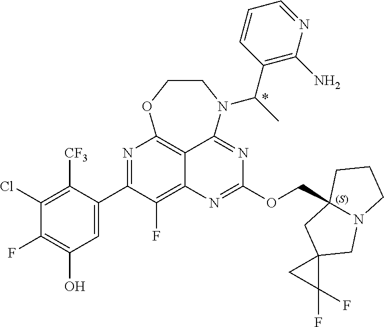 |
| 228 | 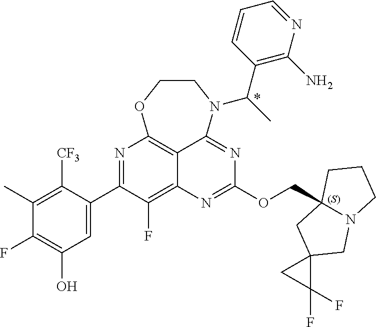 |
| 229 | 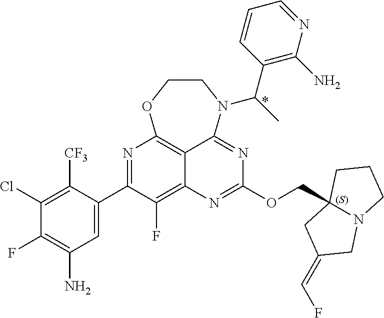 |
| 230 | 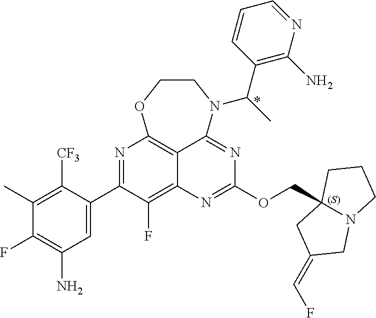 |
| 231 | 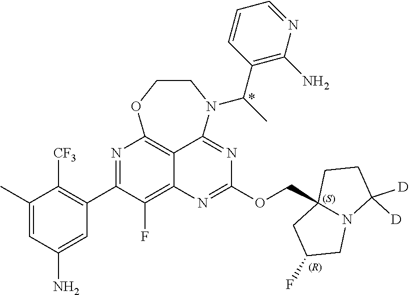 |
| 232 | 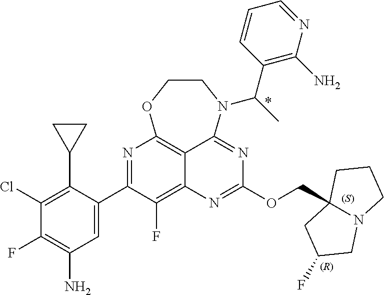 |
| 233 | 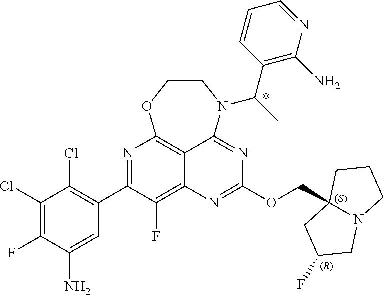 |
| 234 | 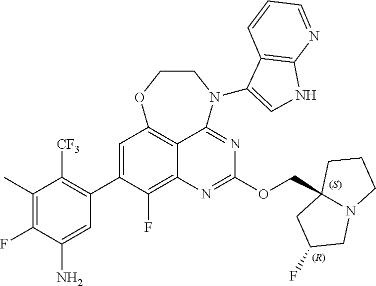 |
| 235 | 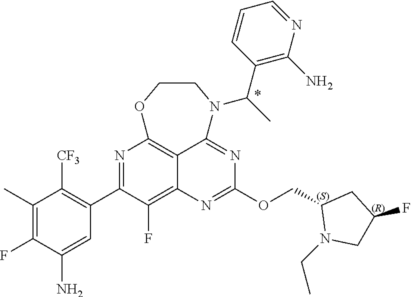 |
| 236 | 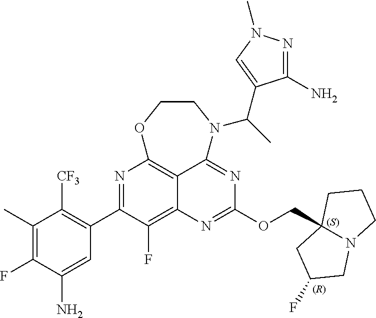 |
| 237 | 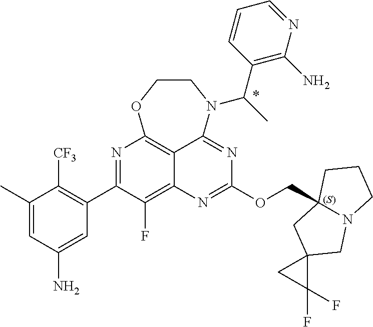 |
| 281 | 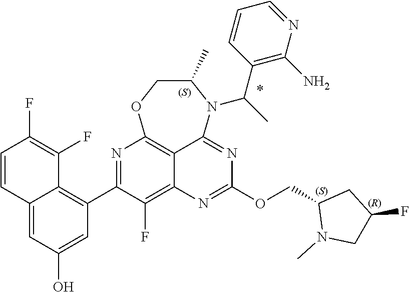 |
| 241 and 242 | 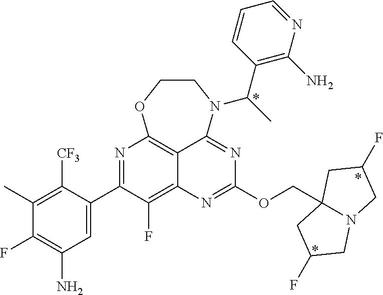 [SMILES: Cc1c(F)c(N)cc(-c2nc3c4c(nc(OCC56CC(F)CN5CC(F)C6)nc4c2F)N(C(C)c2cccnc2N)CCO3)c1C(F)(F)F] + 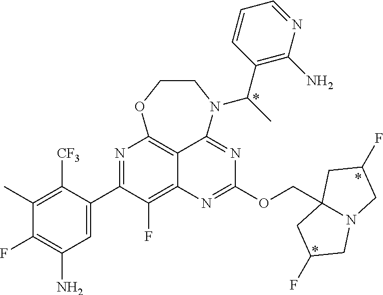 [SMILES: Cc1c(F)c(N)cc(-c2nc3c4c(nc(OCC56CC(F)CN5CC(F)C6)nc4c2F)N(C(C)c2cccnc2N)CCO3)c1C(F)(F)F] 241 & 242 |
| 245 and 246 | 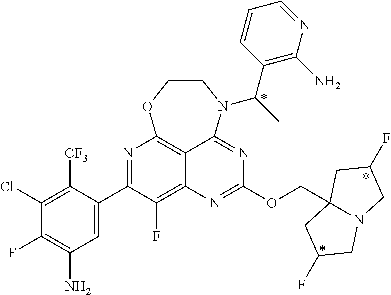 [SMILES: CC(c1cccnc1N)N1CCOc2nc(-c3cc(N)c(F)c(Cl)c3C(F)(F)F)c(F)c3nc(OCC45CC(F)CN4CC(F)C5)nc1c23] + 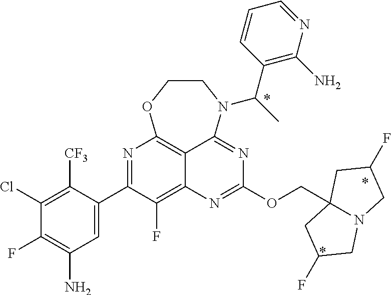 [SMILES: CC(c1cccnc1N)N1CCOc2nc(-c3cc(N)c(F)c(Cl)c3C(F)(F)F)c(F)c3nc(OCC45CC(F)CN4CC(F)C5)nc1c23] 245 & 246 |
| 243 | 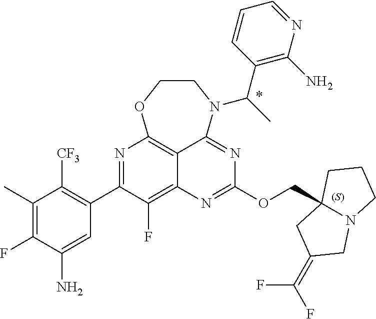 |
| 259 | 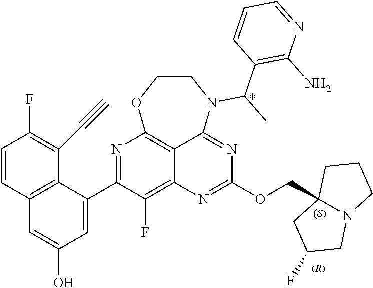 |
| 247 and 248 | 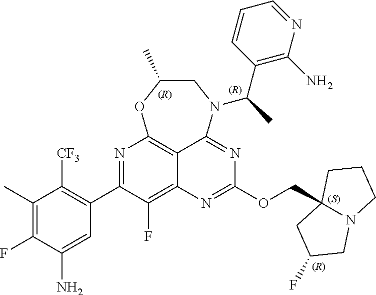 + 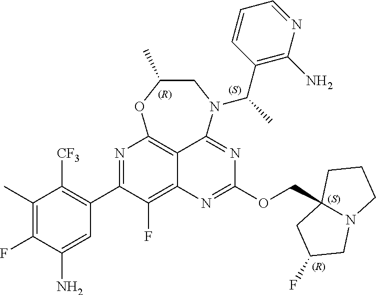 247 & 248 |
| 250 and 263 | 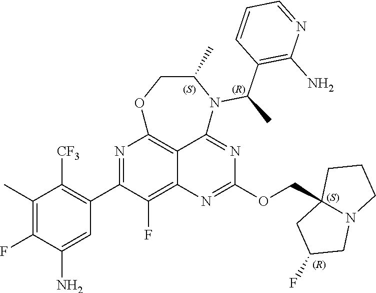 + 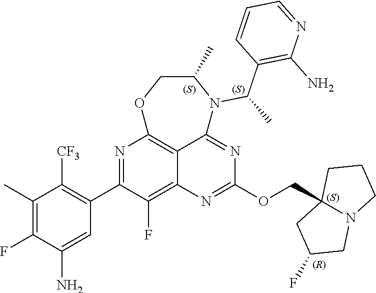 250 & 263 |
| 251 and 252 | 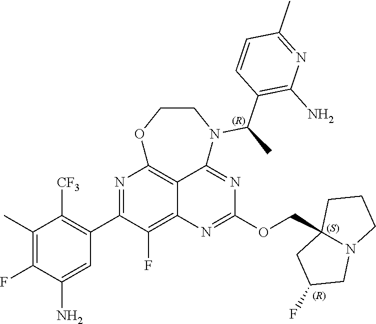 + 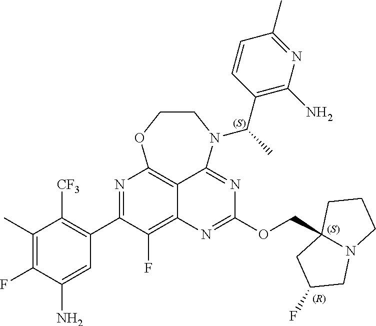 251 & 252 |
| 253 and 254 | 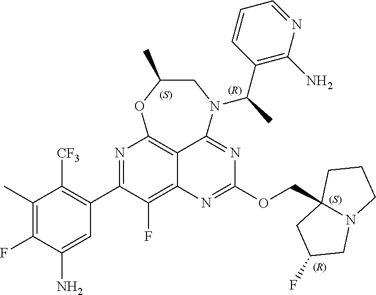 + 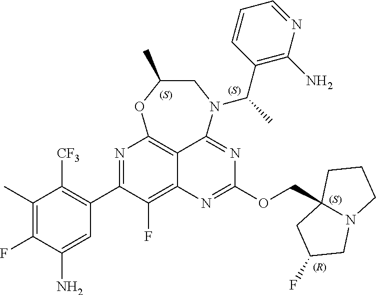 253 & 254 |
| 255 and 256 | 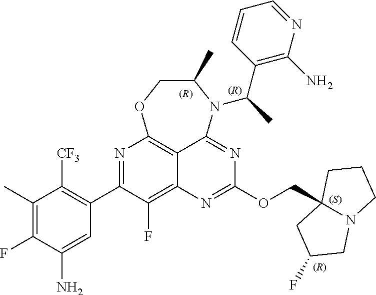 + 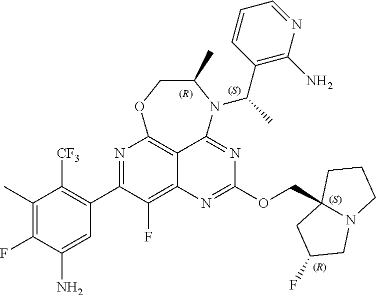 255 & 256 |
| 257 and 258 | 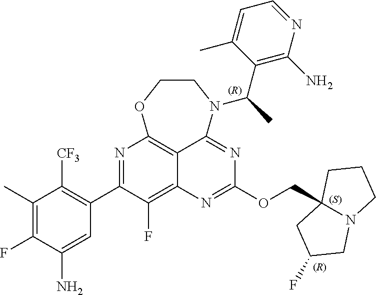 + 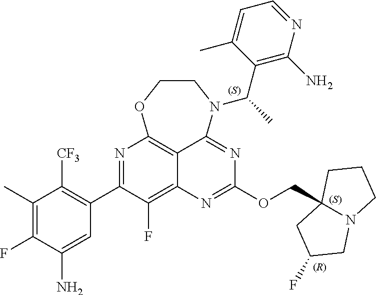 257 & 258 |
| 260 and 261 | 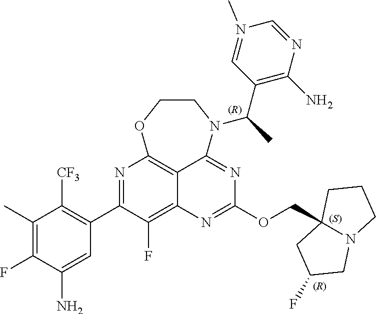 + 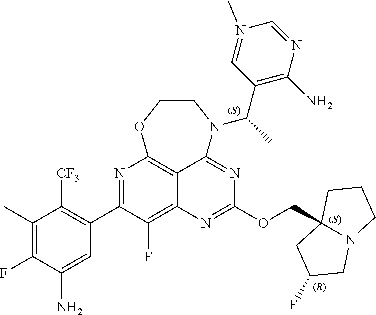 260 & 261 |
| 264 and 265 | 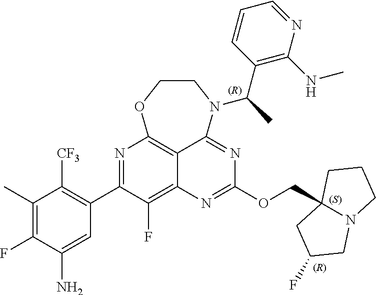 + 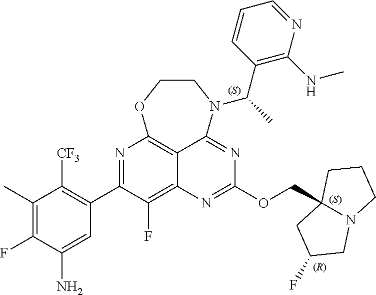 264 & 265 |
| 262 | 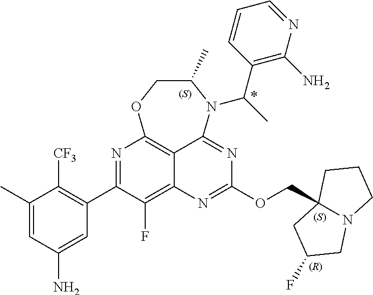 |
| 266 | 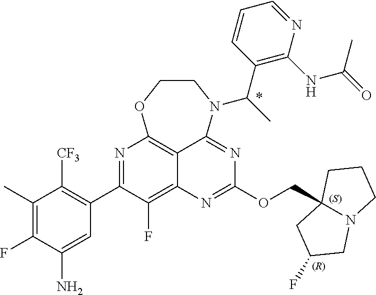 |
| 267 | 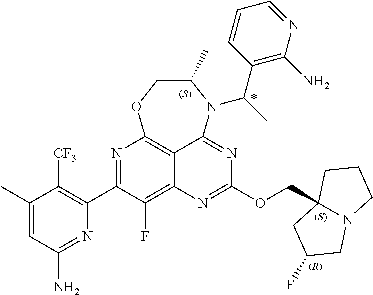 |
| 268 | 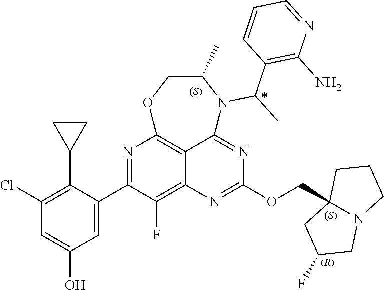 |
| 269 and 272 | 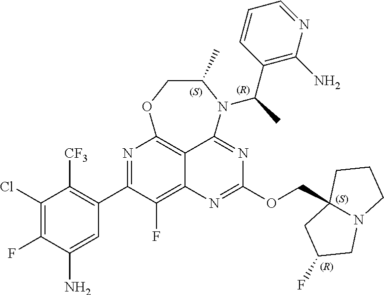 + 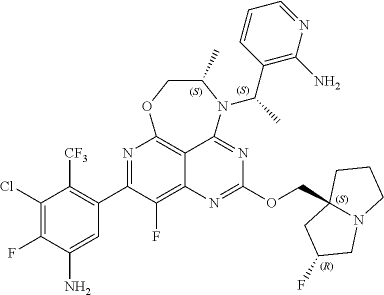 269 & 272 |
| 270 and 271 | 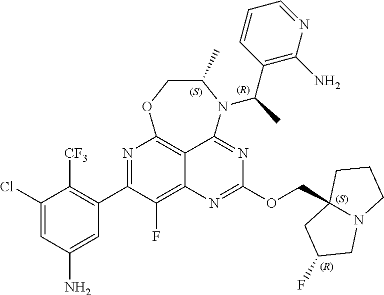 + 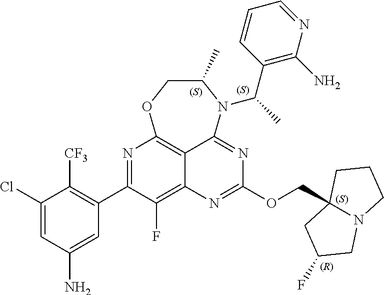 270 & 271 |
| 273 and 274 | 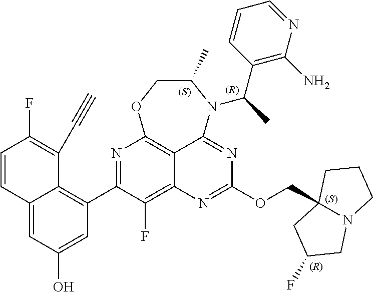 + 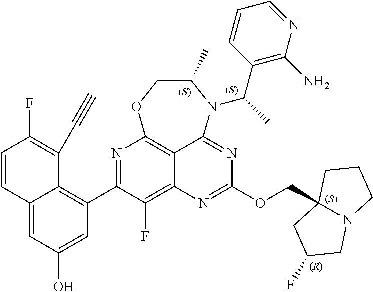 273 & 274 |
| 275 and 276 | 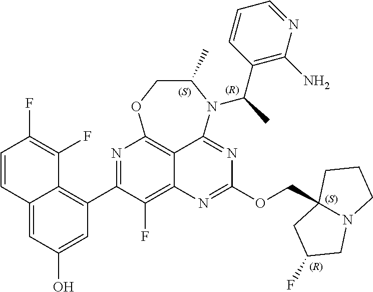 + 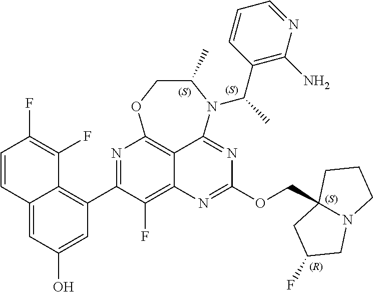 275 & 276 |
| 277 and 278 | 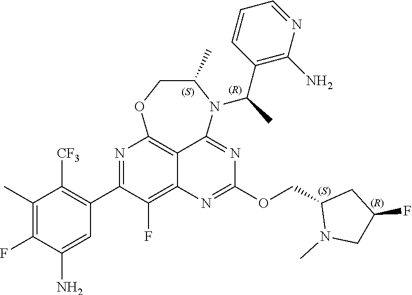 + 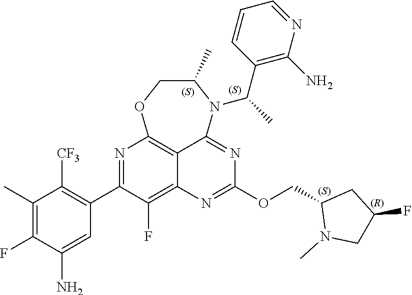 277 & 278 |
| 279 and 280 | 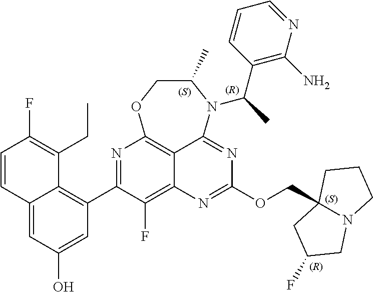 + 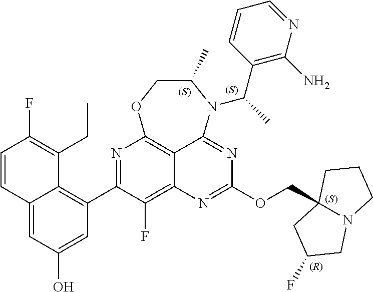 279 & 280 |
| 282 and 283 | 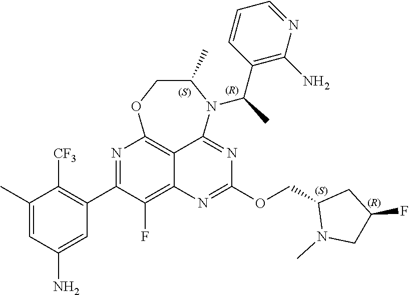 + 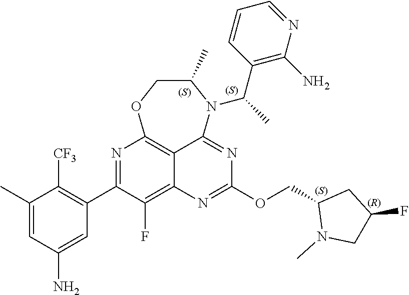 282 & 283 |
| 284 and 285 | 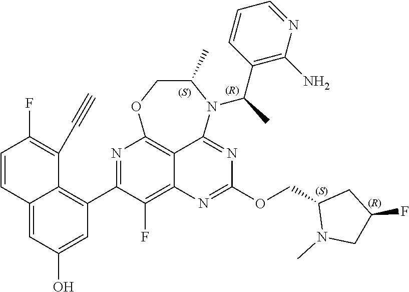 + 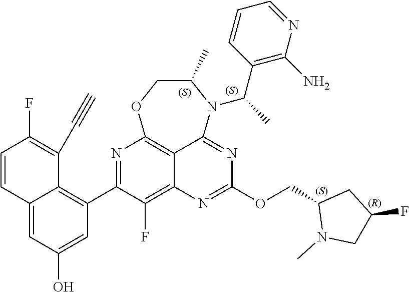 284 & 285 |
| 286 and 287 | 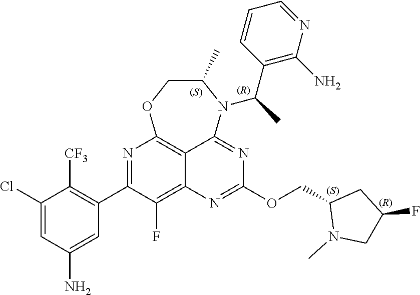 + 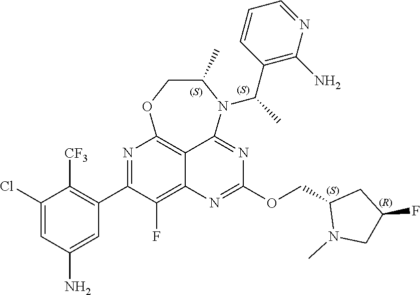 286 & 287 |
| 338 and 339 | 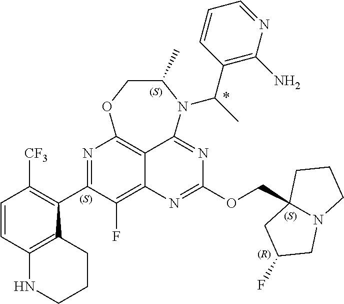 + 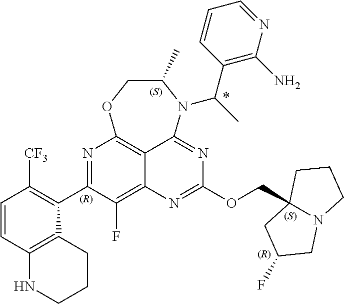 338 & 339 |
| 289 | 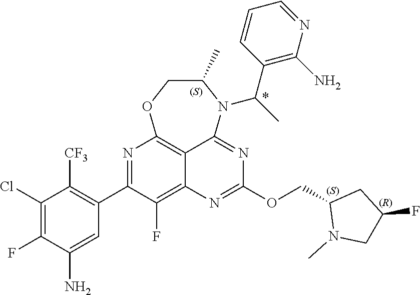 |
| 290 | 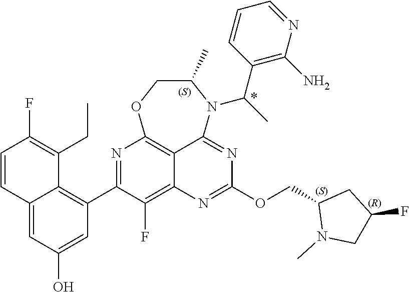 |
| 291 | 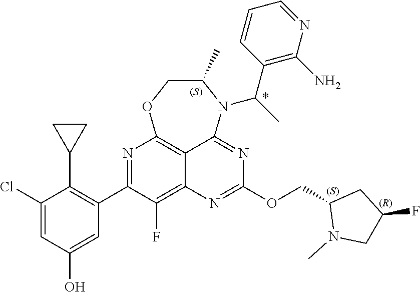 |
| 300 | 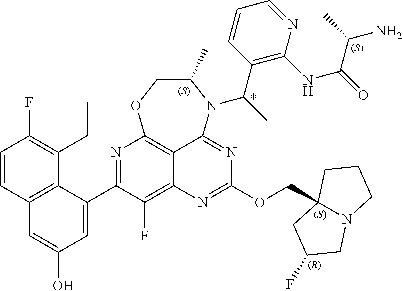 |
| 293 | 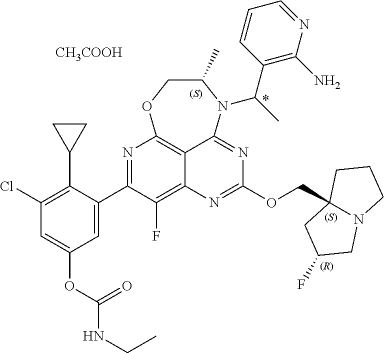 |
| 294 | 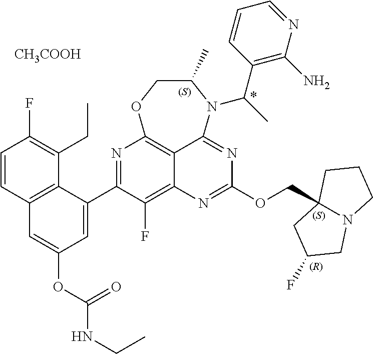 |
| 295 | 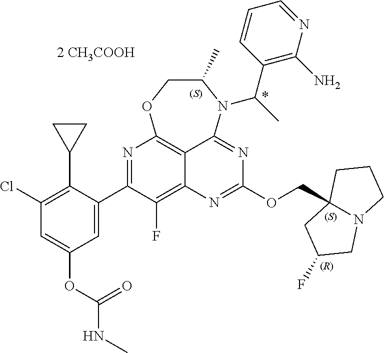 |
| 296 | 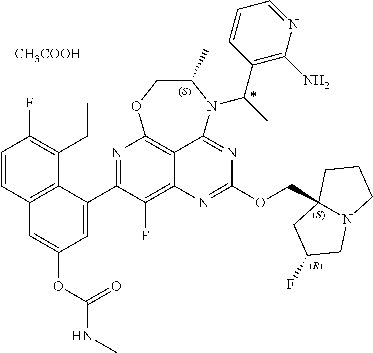 |
| 297 | 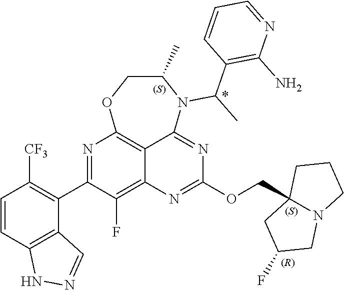 |
| 298 | 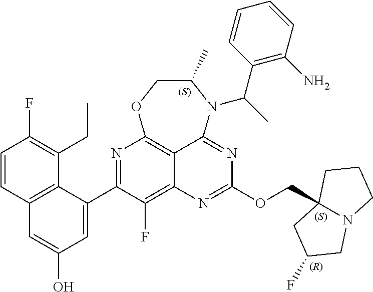 |
| 301 | 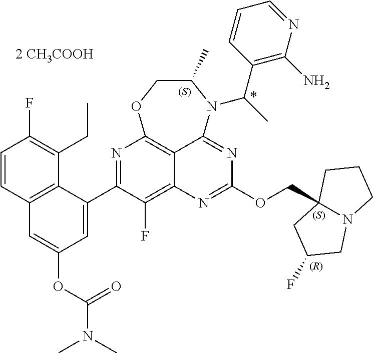 |
| 302 | 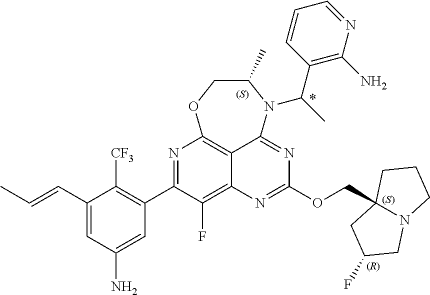 |
| 307 | 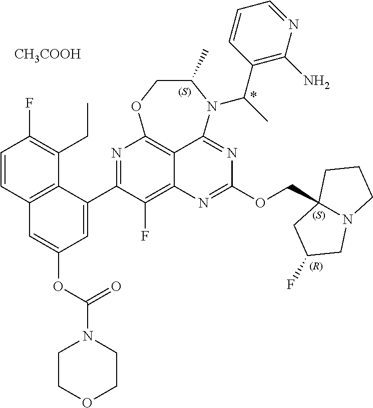 |
| 304 | 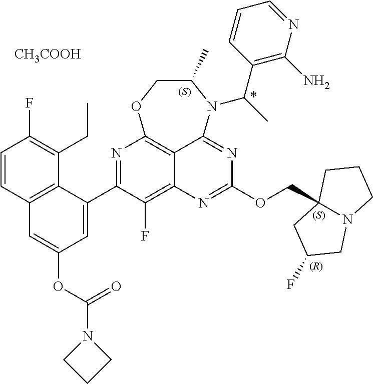 |
| 305 | 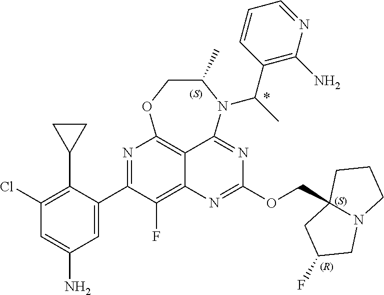 |
| 306 | 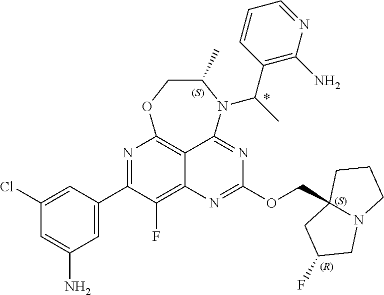 |
| 346 | 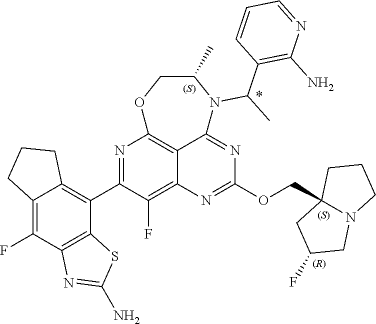 |
| 310 | 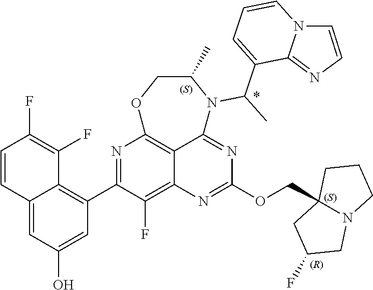 |
| 311 | 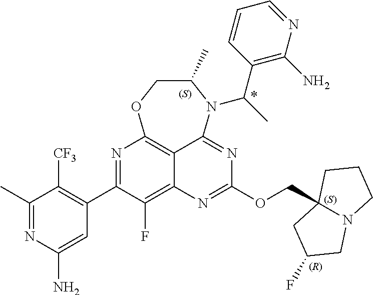 |
| 312 | 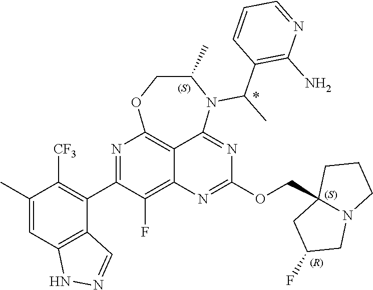 |
| 313 | 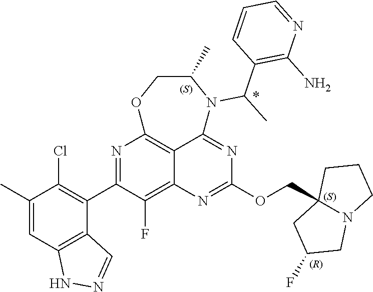 |
| 314 | 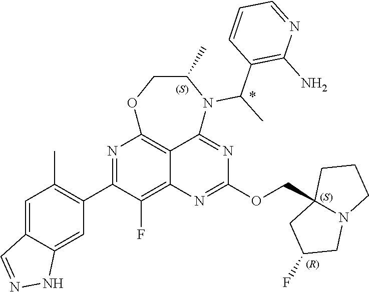 |
| 315 | 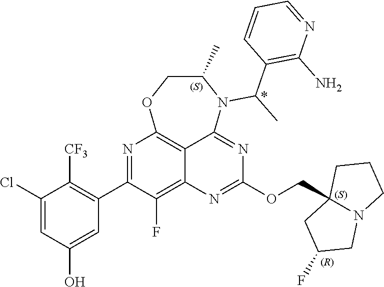 |
| 316 | 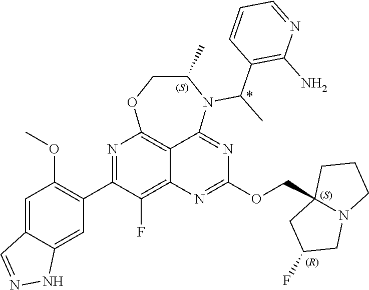 |
| 317 | 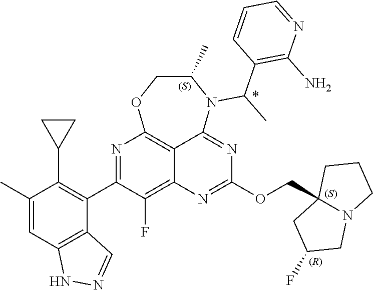 |
| 318 | 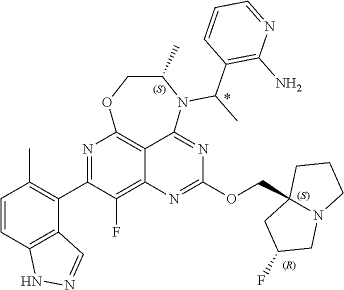 |
| 319 | 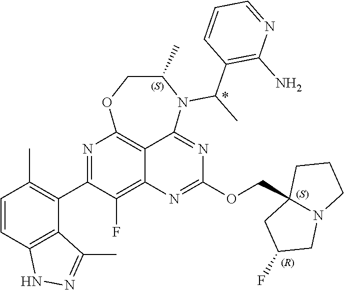 |
| 320 | 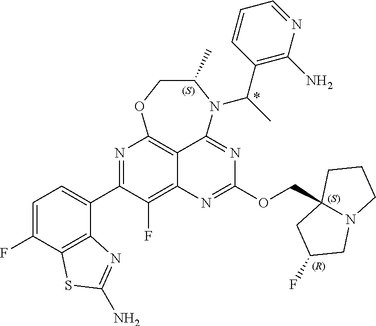 |
| 321 | 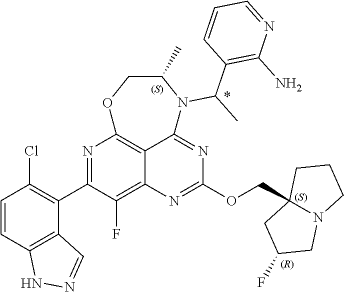 |
| 322 | 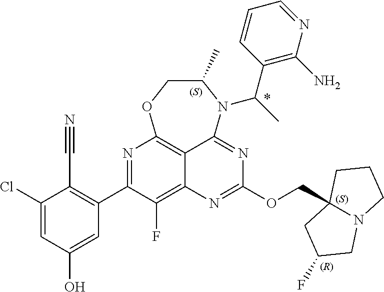 |
| 323 | 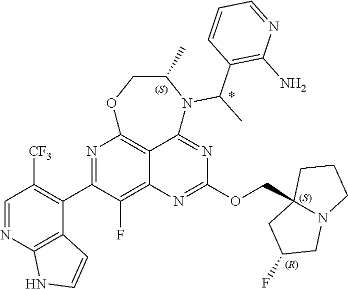 |
| 324 | 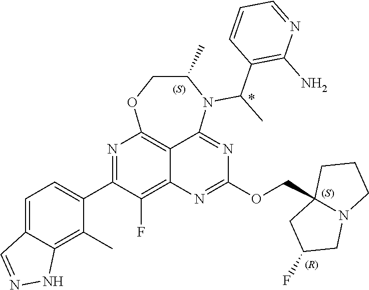 |
| 325 | 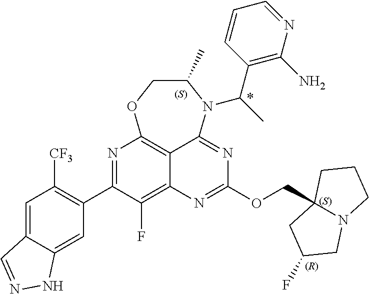 |
| 326 | 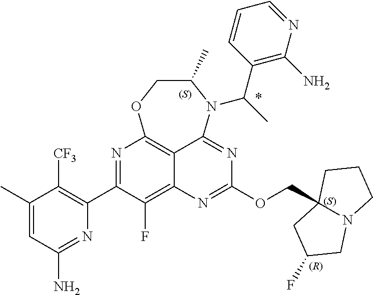 |
| 327 | 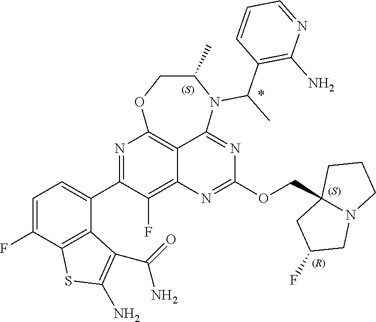 |
| 328 | 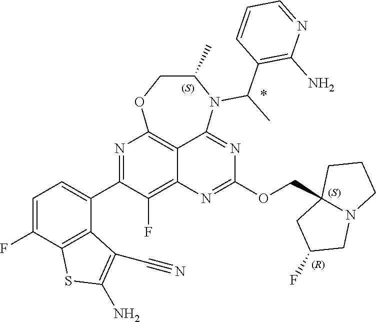 |
| 329 | 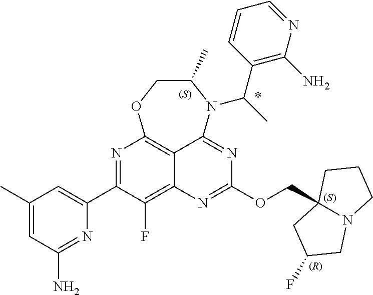 |
| 330 | 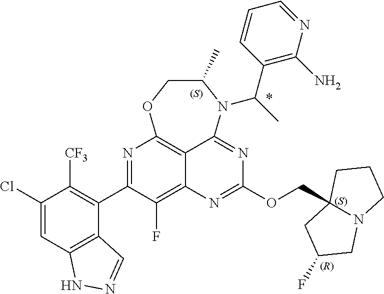 |
| 331 | 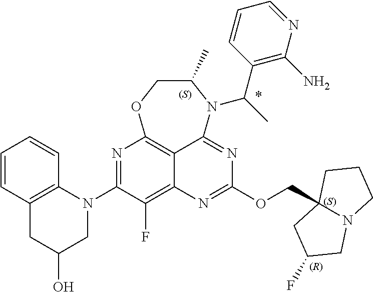 |
| 332 | 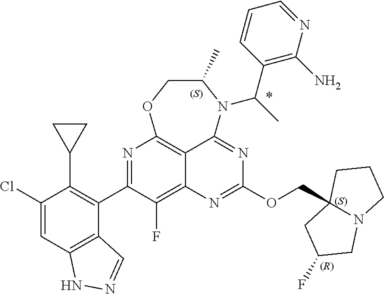 |
| 333 | 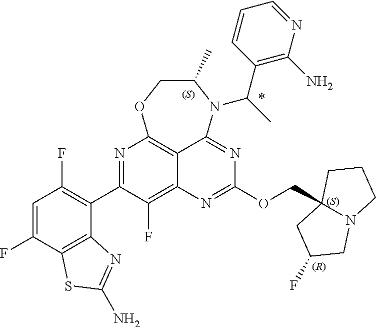 |
| 334 | 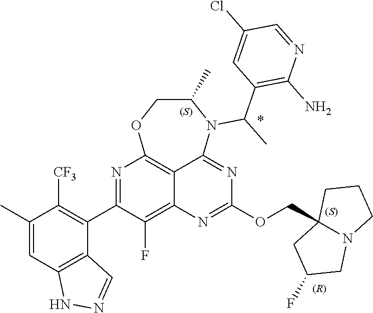 |
| 335 | 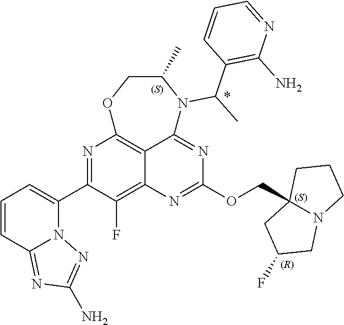 |
| 336 | 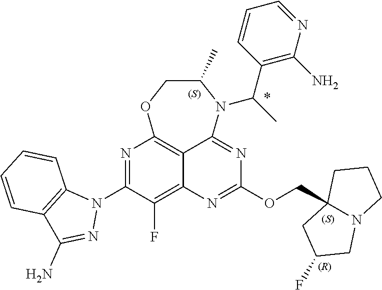 |
| 337 | 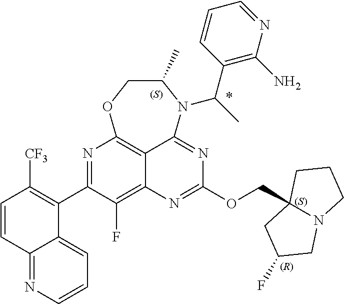 |
| 340 | 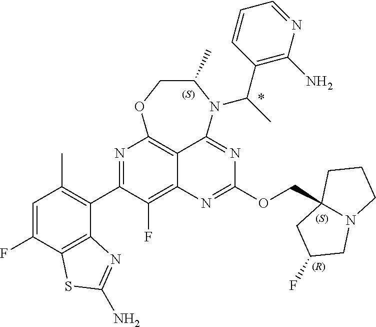 |
| 343 | 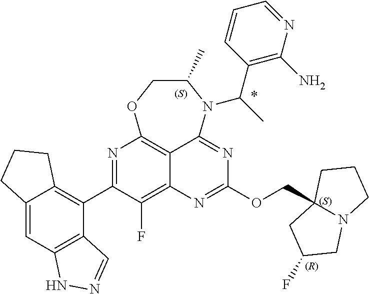 |
| 344 | 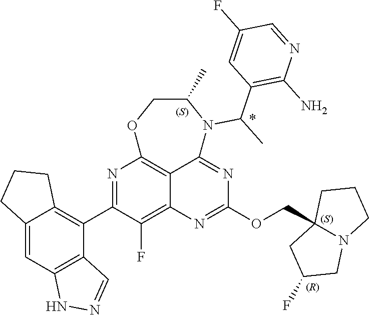 |
| 347 | 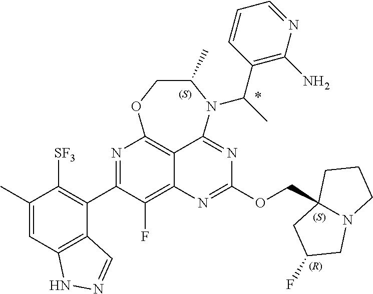 |
| 348 | 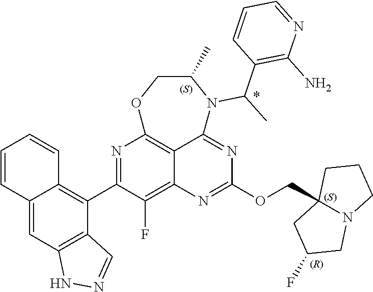 |
| 349 | 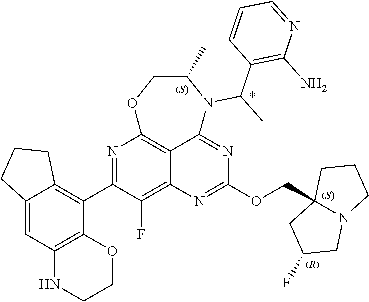 |
| 350 | 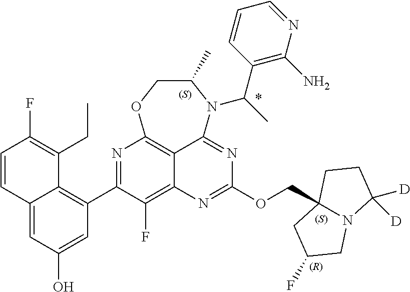 |
| 351 | 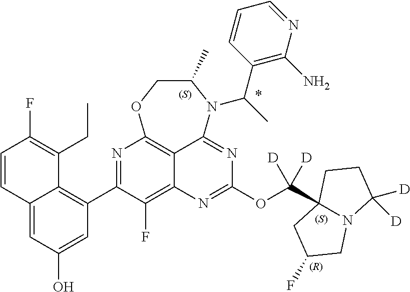 |
| 352 | 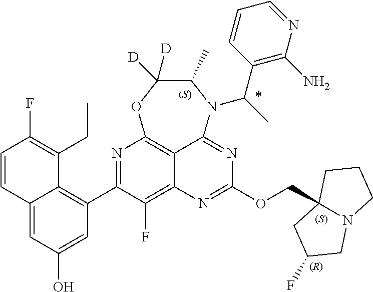 |
| 353 | 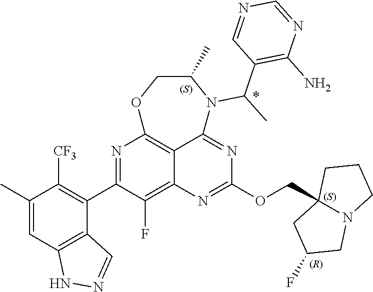 |
| 387 | 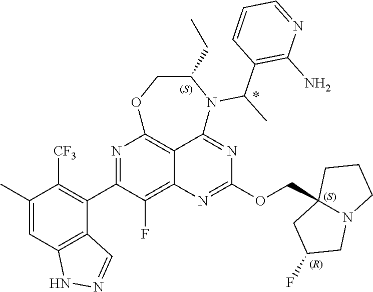 |
| 355 | 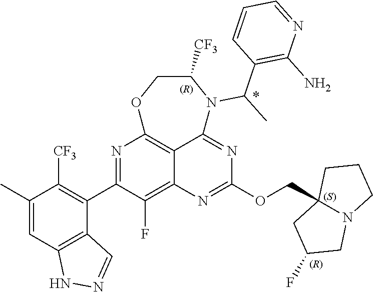 |
| 356 | 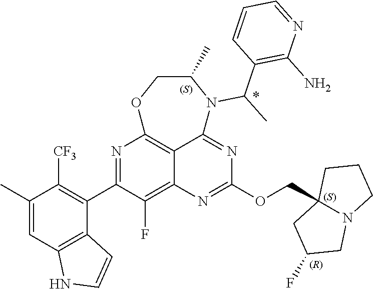 |
| 357 | 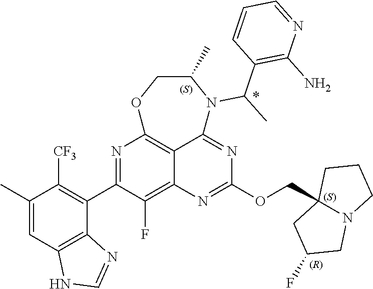 |
| 358 | 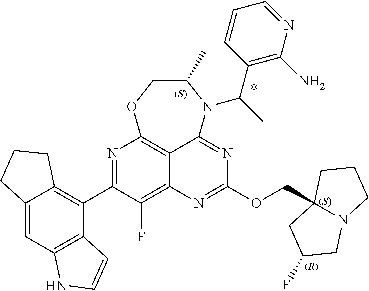 |
| 359 | 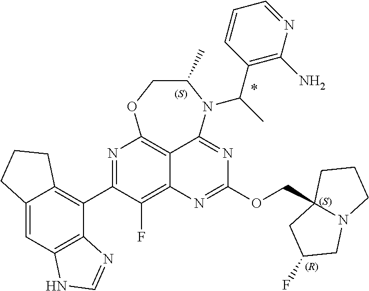 |
| 360 | 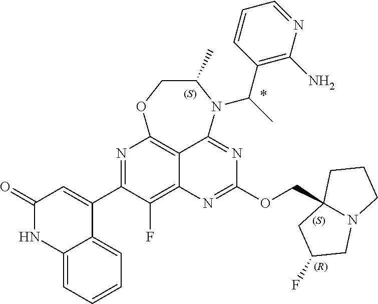 |
| 342 and 364 | 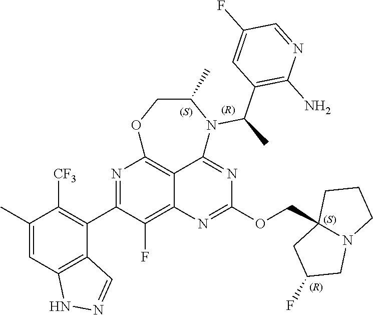 + 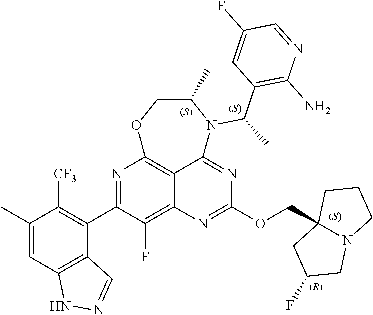 342 & 364 |
| 365 and 365 | 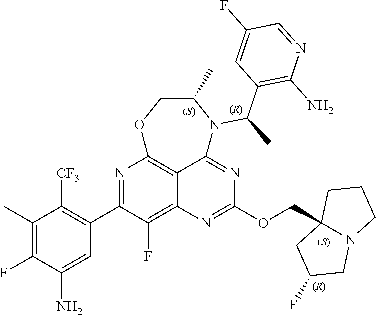 + 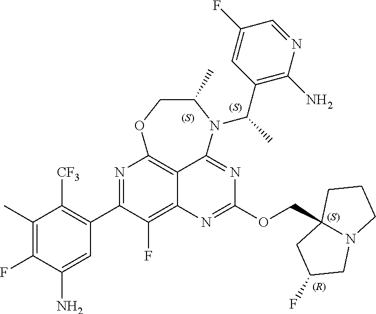 365 & 366 |
| 361 | 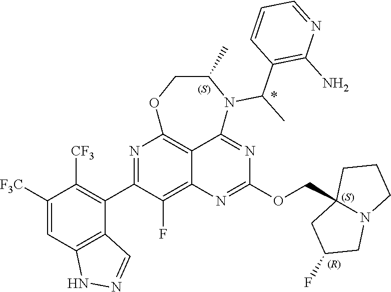 |
| 362 | 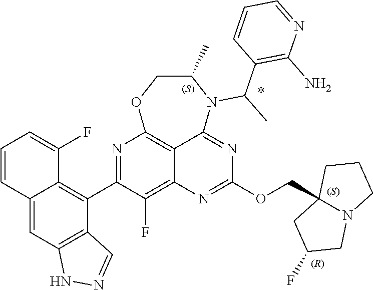 |
| 363 | 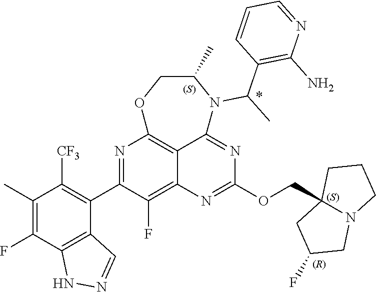 |
| 367 | 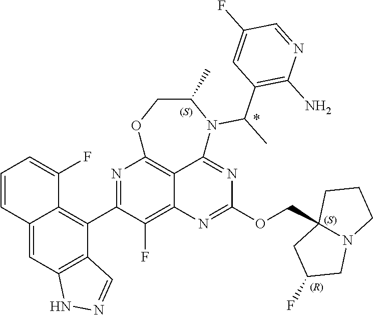 |
| 368 | 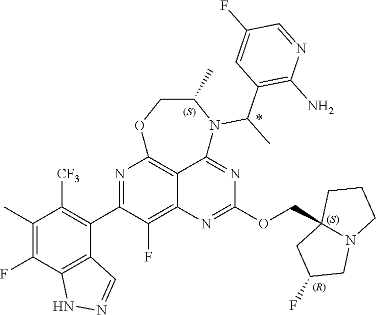 |
| 369 | 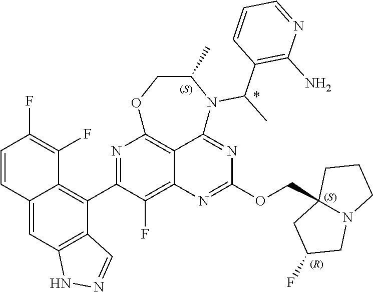 |
| 370 | 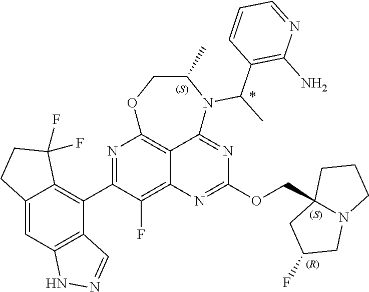 |
| 371 | 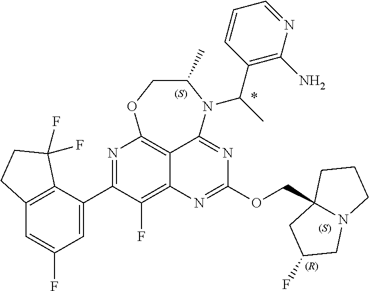 |
| 372 | 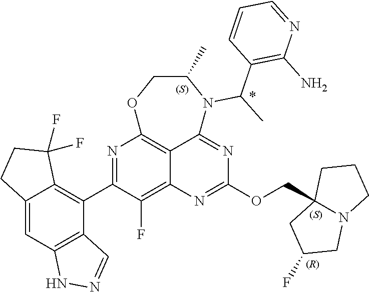 |
| 373 | 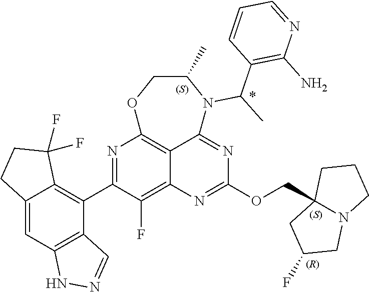 |
| 374 | 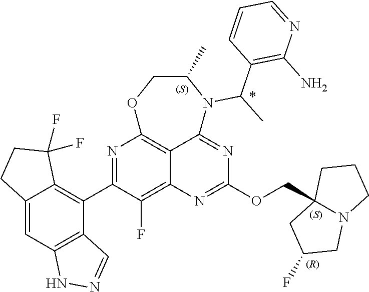 |
| 375 | 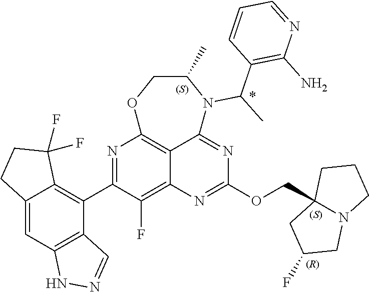 |
| 376 | 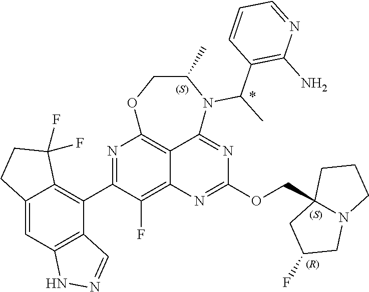 |
| 377 and 378 | 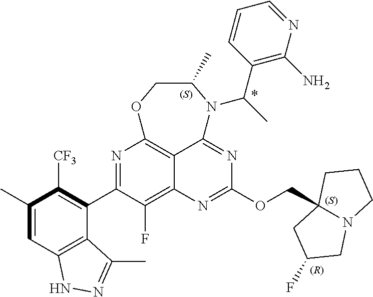 + 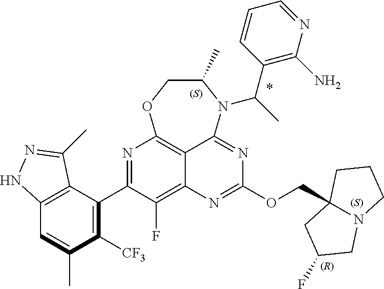 377 & 378 |
| 379 and 380 | 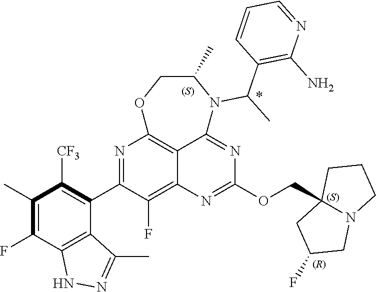 + 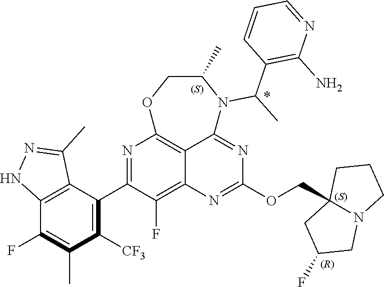 379 & 380 |
| 381 and 382 | 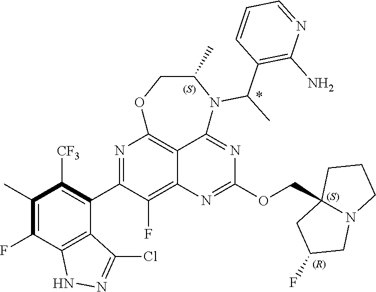 + 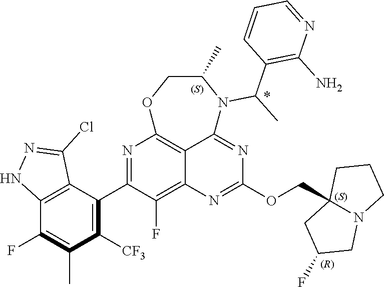 381 & 382 |
| 383 | 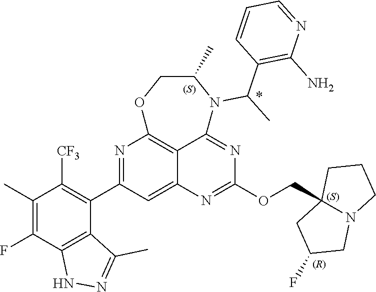 |
| 384 | 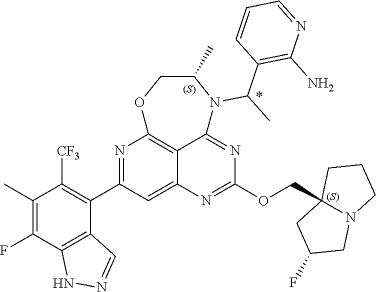 |
| 385 | 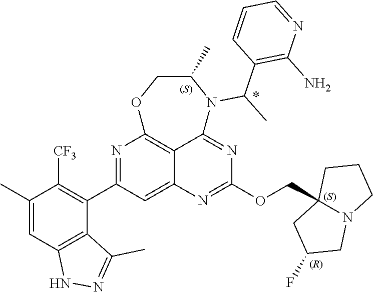 |
| 386 | 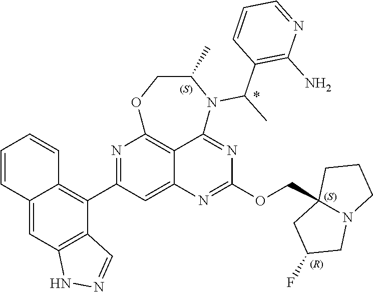 |
| 388 | 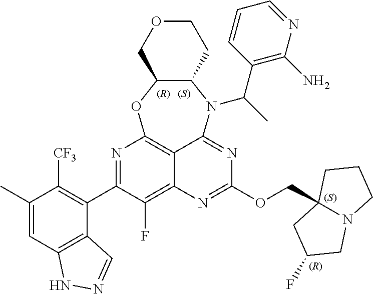 |
| 389 | 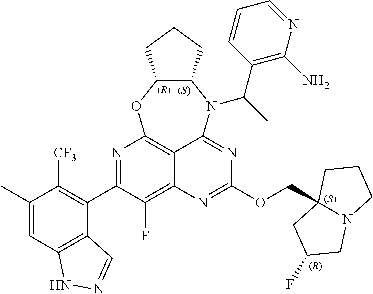 |
| 390 | 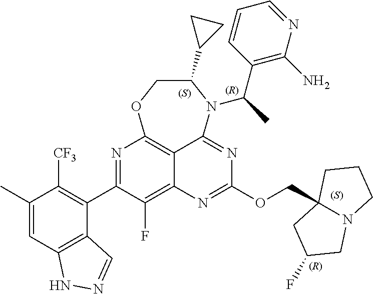 |
| 391 | 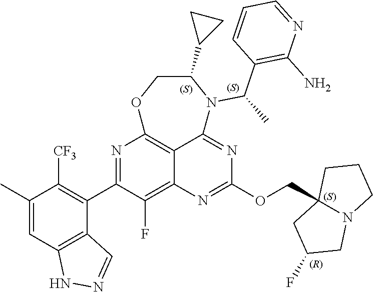 |
| 392 | 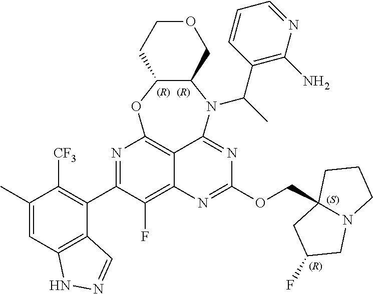 |
| 393 | 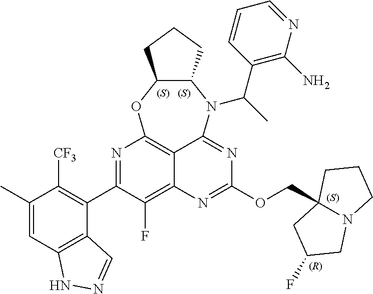 |
| 394 | 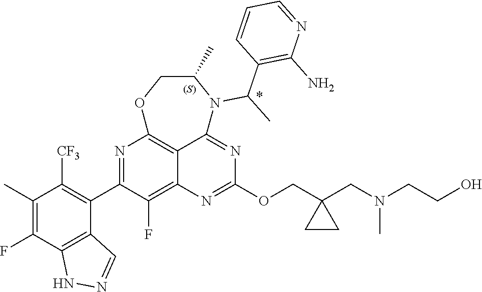 |
| 395 | 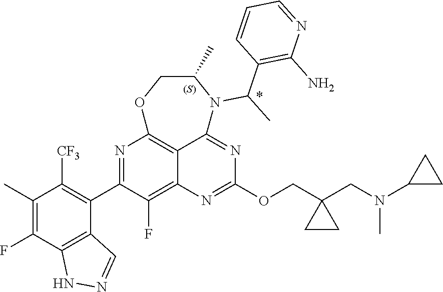 |
| 396 | 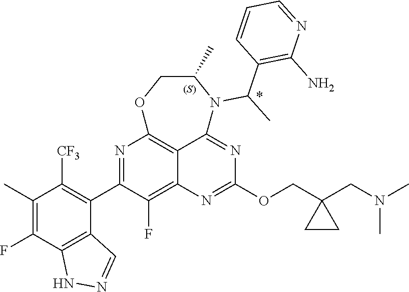 |
| 397 | 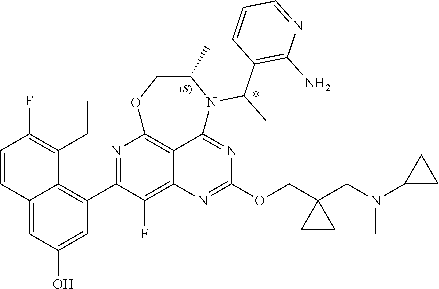 |
| 398 | 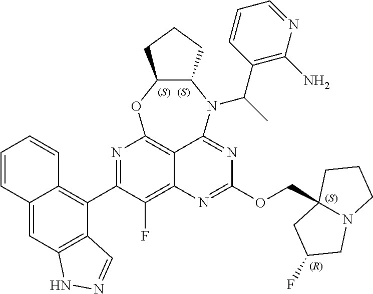 |
| 401 | 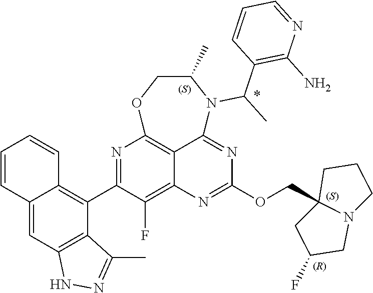 |
| 399 and 400 | 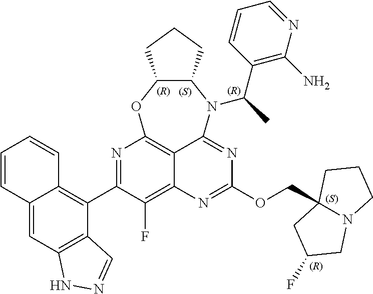 + 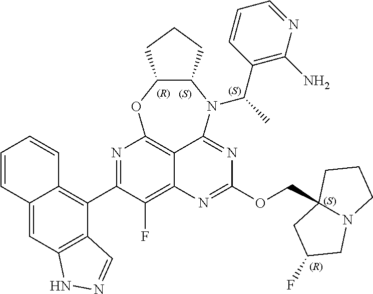 399 & 400 |
| 402 | 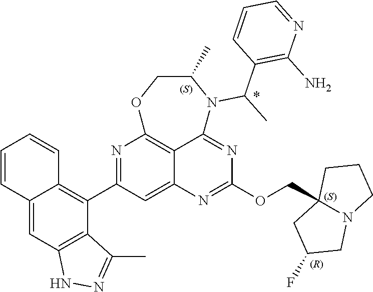 |
| 403 | 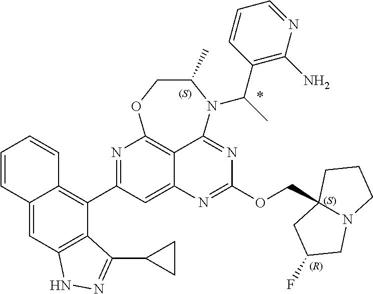 |
| 404 | 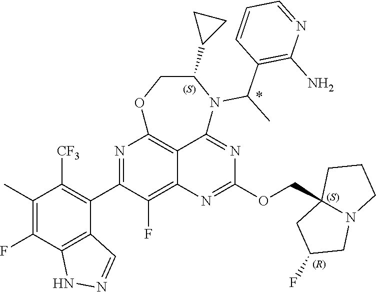 |
| 405 | 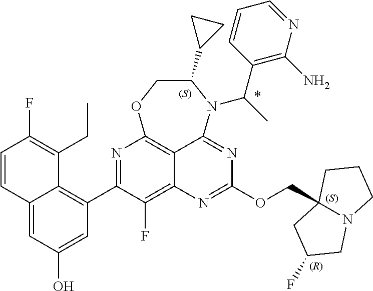 |
| 406 | 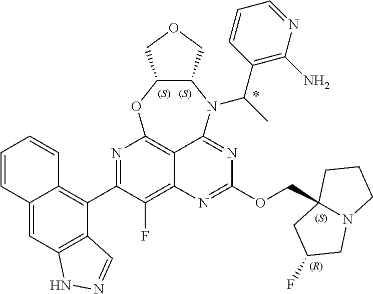 |
| 407 | 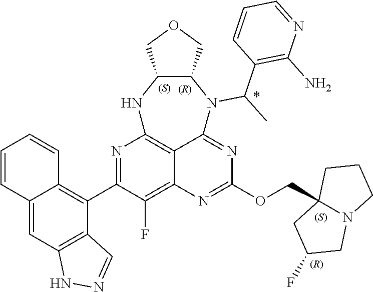 |
| 408 | 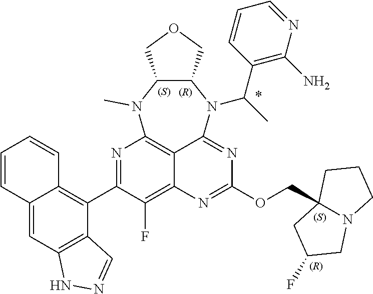 |
| 409 | 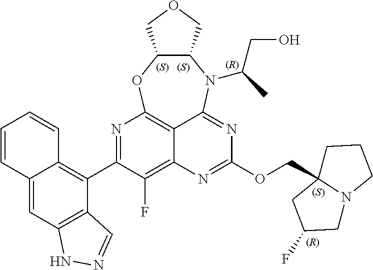 |
| 410 | 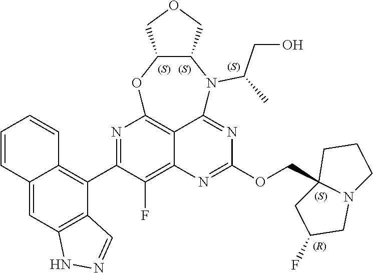 |
| 411 | 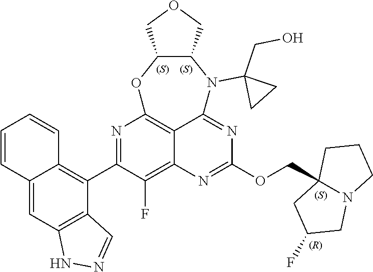 |
| 412 | 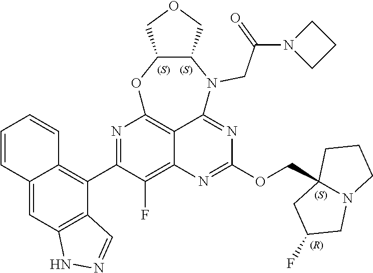 |
| 413 | 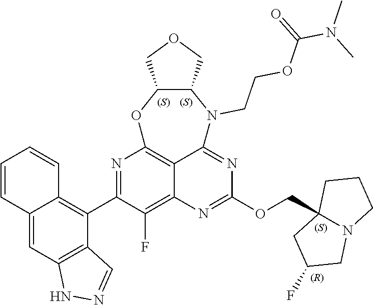 |
| 414 | 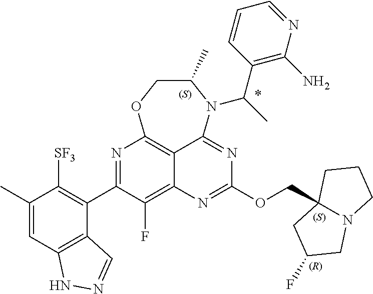 |
| 415 | 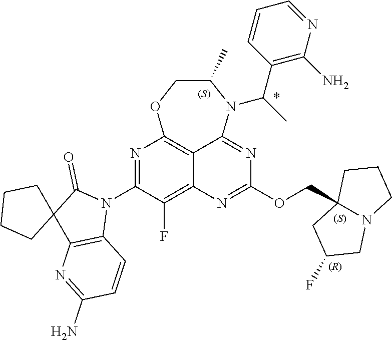 |
| 416 | 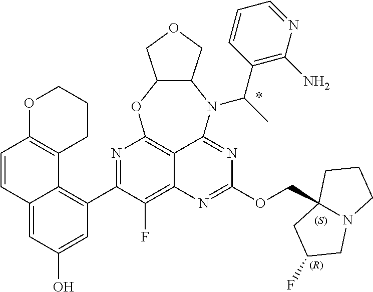 |
| 417 | 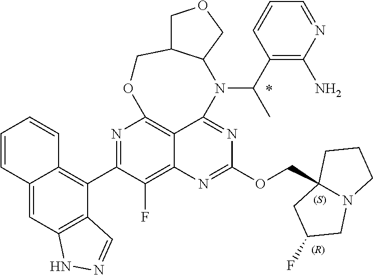 |
| 418 | 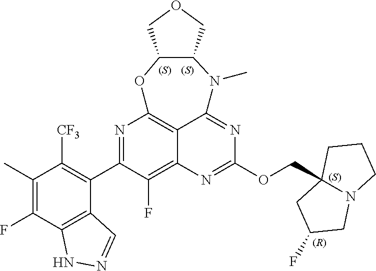 |
| 419 | 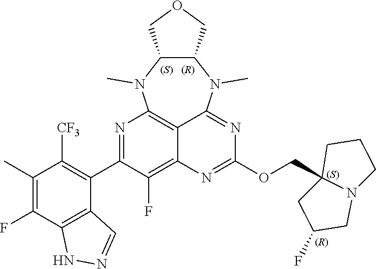 |
| 420 | 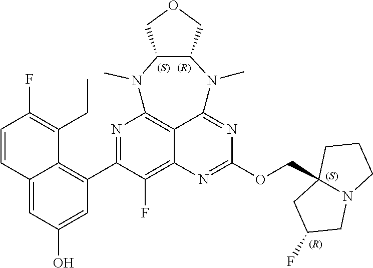 |
| 421 | 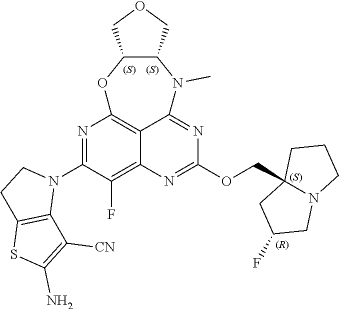 |
| 422 | 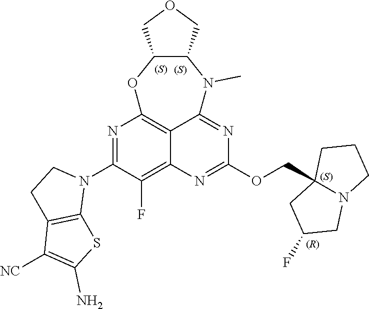 |
| 423 | 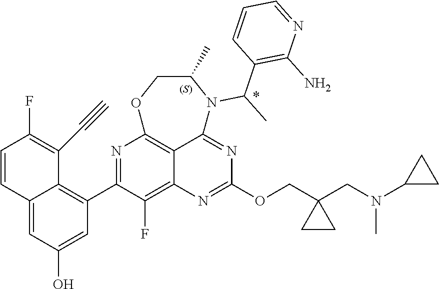 |
| 424 | 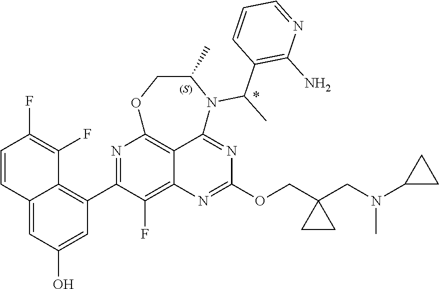 |
| 425 | 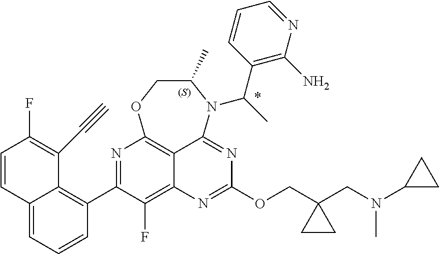 |
| 426 | 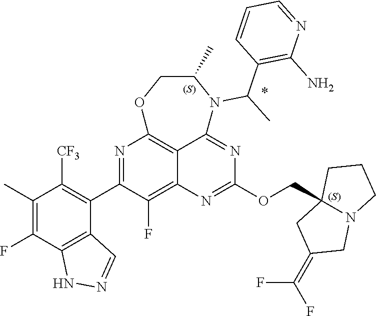 |
| 427 | 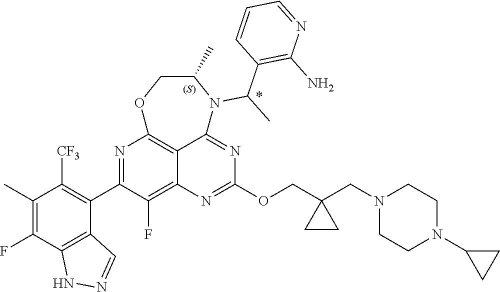 |
| 428 | 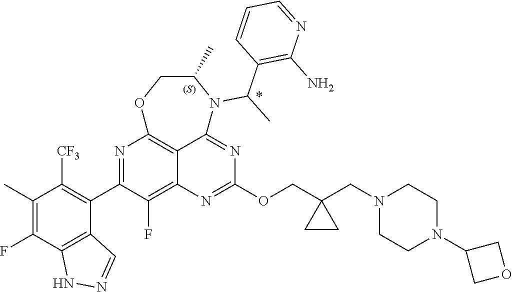 |
| 429 | 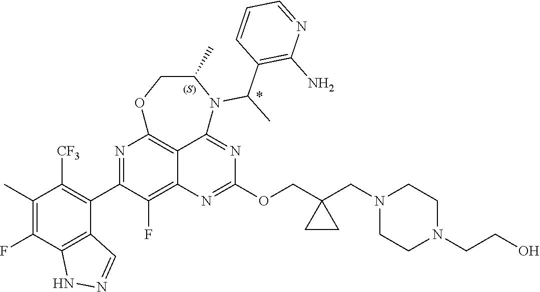 |
| 430 | 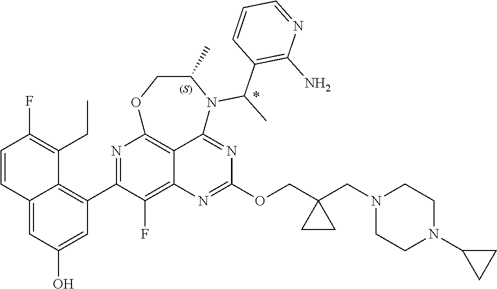 |

Embodiment 27. A pharmaceutical composition, comprising the compound and/or the pharmaceutically acceptable salt thereof according to any one of embodiments 1-26, and optionally comprising a pharmaceutically acceptable excipient.

Embodiment 28. A method of in vivo or in vitro inhibiting the activity of mutant KRAS proteins, comprising contacting the mutant KRAS proteins with an effective amount of the compound and/or the pharmaceutically acceptable salt thereof according to any one of embodiments 1-26.

Embodiment 29. A method of treating or preventing a disease in a subject, comprising administering to the subject in need thereof an effective amount of the compound and/or the pharmaceutically acceptable salt thereof according to any one of embodiments 1-26, wherein the disease is characterized by containing KRAS mutations or KRAS gene amplification; the KRAS mutations are preferably KRAS G12C mutation, KRAS G12D mutation, KRAS G12V mutation, KRAS G13D mutation, and/or KRAS Q61H mutation; the KRAS gene amplification is preferably wild-type KRAS gene amplification; the disease is preferably cancer; and the cancer is preferably a solid tumor or hematologic malignancy; the cancer is more preferably selected from lung cancer (such as lung adenocarcinoma, non-small cell lung cancer, and small cell lung cancer), colorectal cancer, pancreatic cancer, colon cancer, rectal cancer, thyroid carcinoma, esophageal cancer (such as esophageal adenocarcinoma), cholangiocarcinoma, gallbladder carcinoma, head and neck cancer, breast cancer, endometrial cancer, prostate cancer, ovarian cancer, cervical cancer, neuroblastoma, melanoma, brain tumor, gastric cancer (such as chromosomal instability gastric cancer, and gastric adenocarcinoma), bladder cancer, liver cancer, renal cancer, bone cancer, sarcoma, adrenal carcinoma, leukemia, lymphoma and myeloma; and the cancer is further preferably selected from lung cancer (such as lung adenocarcinoma, non-small cell lung cancer, and small cell lung cancer), colorectal cancer, pancreatic cancer, colon cancer, esophageal cancer (such as esophageal adenocarcinoma), and gastric cancer (such as chromosomal instability gastric cancer and gastric adenocarcinoma).

Embodiment 30. Use of the compound and/or the pharmaceutically acceptable salt thereof according to any one of embodiments 1-26 in the manufacture of a medicament for treating or preventing a disease containing KRAS mutations or KRAS gene amplification, wherein the KRAS mutations are preferably KRAS G12C mutation, KRAS G12D mutation, KRAS G12V mutation, KRAS G13D mutation, and/or KRAS Q61H mutation; the KRAS gene amplification is preferably wild-type KRAS gene amplification; the disease is preferably cancer; and the cancer is preferably a solid tumor or hematologic malignancy; the cancer is more preferably selected from lung cancer (such as lung adenocarcinoma, non-small cell lung cancer, and small cell lung cancer), colorectal cancer, pancreatic cancer, colon cancer, rectal cancer, thyroid carcinoma, esophageal cancer (such as esophageal adenocarcinoma), cholangiocarcinoma, gallbladder carcinoma, head and neck cancer, breast cancer, endometrial cancer, prostate cancer, ovarian cancer, cervical cancer, neuroblastoma, melanoma, brain tumor, gastric cancer (such as chromosomal instability gastric cancer, and gastric adenocarcinoma), bladder cancer, liver cancer, renal cancer, bone cancer, sarcoma, adrenal carcinoma, leukemia, lymphoma and myeloma; and the cancer is further preferably selected from lung cancer (such as lung adenocarcinoma, non-small cell lung cancer, and small cell lung cancer), colorectal cancer, pancreatic cancer, colon cancer, esophageal cancer (such as esophageal adenocarcinoma), and gastric cancer (such as chromosomal instability gastric cancer and gastric adenocarcinoma).

Embodiment 31. The compound and/or the pharmaceutically acceptable salt thereof according to any one of embodiments 1-26, for use as a medicament.

Embodiment 32. The compound and/or the pharmaceutically acceptable salt thereof according to any one of embodiments 1-26, for use in treating or preventing a disease containing KRAS mutations or KRAS gene amplification, wherein the disease is preferably cancer; the KRAS mutations are preferably KRAS G12C mutation, KRAS G12D mutation, KRAS G12V mutation, KRAS G13D mutation, and/or KRAS Q61H mutation; the KRAS gene amplification is preferably wild-type KRAS gene amplification; and the cancer is preferably a solid tumor or hematologic malignancy; the cancer is more preferably selected from lung cancer (such as lung adenocarcinoma, non-small cell lung cancer, and small cell lung cancer), colorectal cancer, pancreatic cancer, colon cancer, rectal cancer, thyroid carcinoma, esophageal cancer (such as esophageal adenocarcinoma), cholangiocarcinoma, gallbladder carcinoma, head and neck cancer, breast cancer, endometrial cancer, prostate cancer, ovarian cancer, cervical cancer, neuroblastoma, melanoma, brain tumor, gastric cancer (such as chromosomal instability gastric cancer, and gastric adenocarcinoma), bladder cancer, liver cancer, renal cancer, bone cancer, sarcoma, adrenal carcinoma, leukemia, lymphoma and myeloma; and the cancer is further preferably selected from lung cancer (such as lung adenocarcinoma, non-small cell lung cancer, and small cell lung cancer), colorectal cancer, pancreatic cancer, colon cancer, esophageal cancer (such as esophageal adenocarcinoma), and gastric cancer (such as chromosomal instability gastric cancer and gastric adenocarcinoma).

Embodiment 33. A pharmaceutical combination, comprising the compound and/or the pharmaceutically acceptable salt thereof according to any one of embodiments 1-26, and at least one additional therapeutic agent, wherein the additional therapeutic agent is preferably selected from an anti-neoplastic active agent, an anti-inflammatory agent or an immunomodulator, wherein the anti-neoplastic active agent includes a chemotherapeutic agent, an immune checkpoint inhibitor or agonist, and a targeted therapeutic agent.

Embodiment 34. A compound of formula (II):

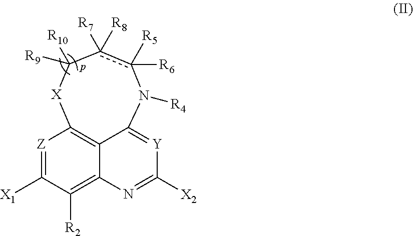

or a pharmaceutically acceptable salt thereof, or a solvate, a racemic mixture, an enantiomer, a diastereomer or a tautomer thereof, wherein
R₂, R₄, R₅, R₆, R₇, R₈, R₉, R₁₀, X, Y, Z and p are as defined in any one of embodiments 1-25;
X₁ is halogen; and
X₂ is hydrogen, halogen, —S(C₁₋₆ alkyl) or —S(O)(C₁₋₆ alkyl).

Embodiment 35. The compound or the pharmaceutically acceptable salt thereof, or the solvate, the racemic mixture, the enantiomer, the diastereomer or the tautomer thereof according to embodiment 34, wherein the compound is a compound of

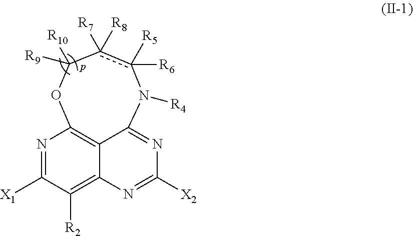

wherein

X₁ is halogen; and preferably, X₁ is chlorine; and
X₂ is hydrogen, halogen, —S(C₁₋₆ alkyl) or —S(O)(C₁₋₆ alkyl); preferably, X₂ is hydrogen, chlorine, —S(CH₃) or —S(O)(CH₃); and more preferably, X₂ is hydrogen, —S(CH₃) or —S(O)(CH₃).

Embodiment 36. The compound or the pharmaceutically acceptable salt thereof, or the solvate, the racemic mixture, the enantiomer, the diastereomer or the tautomer thereof according to embodiment 35, which is selected from:

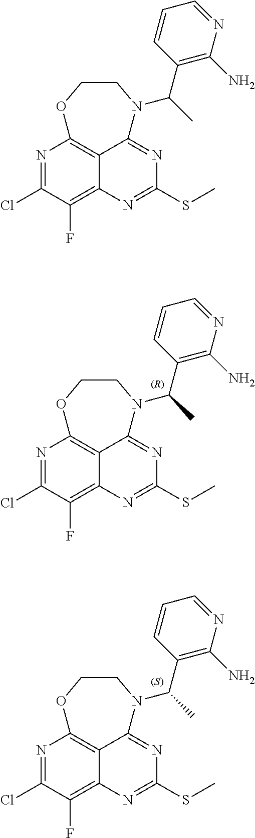

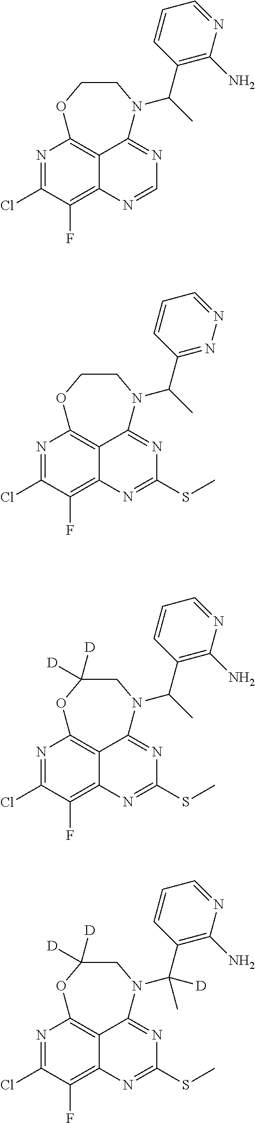 [SMILES: CC(c1cccnc1N)N1CCOc2nc(Cl)c(F)c3ncnc1c23 ; CSc1nc2c3c(nc(Cl)c(F)c3n1)OCCN2C(C)c1cccnn1 ; [2H]C1([2H])CN(C(C)c2cccnc2N)c2nc(SC)nc3c(F)c(Cl)nc(c23)O1 ; [2H]C1([2H])CN(C([2H])(C)c2cccnc2N)c2nc(SC)nc3c(F)c(Cl)nc(c23)O1]

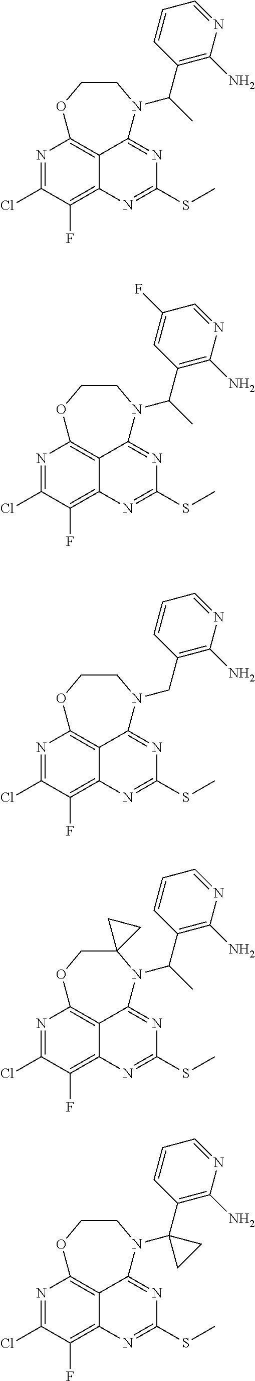 [SMILES: CSc1nc2c3c(nc(Cl)c(F)c3n1)OCCN2C(C)c1cccnc1N ; CSc1nc2c3c(nc(Cl)c(F)c3n1)OCCN2C(C)c1cc(F)cnc1N ; CSc1nc2c3c(nc(Cl)c(F)c3n1)OCCN2Cc1cccnc1N ; CSc1nc2c3c(nc(Cl)c(F)c3n1)OCC1(CC1)N2C(C)c1cccnc1N ; CSc1nc2c3c(nc(Cl)c(F)c3n1)OCCN2C1(c2cccnc2N)CC1]

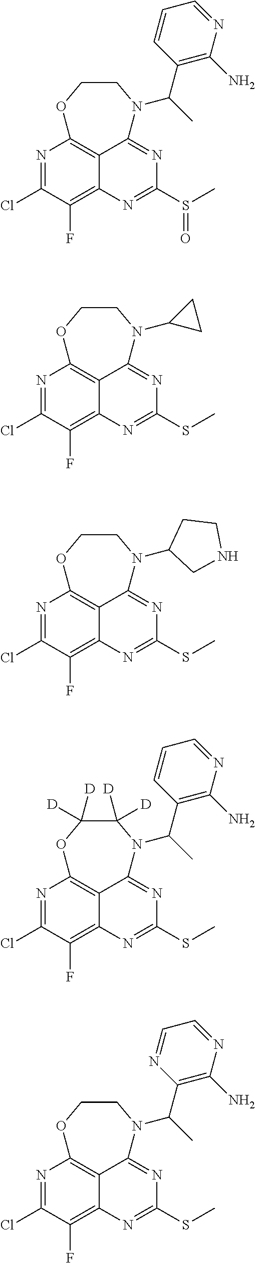 [SMILES: CC(c1cccnc1N)N1CCOc2nc(Cl)c(F)c3nc(S(C)=O)nc1c23 ; CSc1nc2c3c(nc(Cl)c(F)c3n1)OCCN2C1CC1 ; CSc1nc2c3c(nc(Cl)c(F)c3n1)OCCN2C1CCNC1 ; [2H]C1([2H])Oc2nc(Cl)c(F)c3nc(SC)nc(c23)N(C(C)c2cccnc2N)C1([2H])[2H] ; CSc1nc2c3c(nc(Cl)c(F)c3n1)OCCN2C(C)c1nccnc1N]

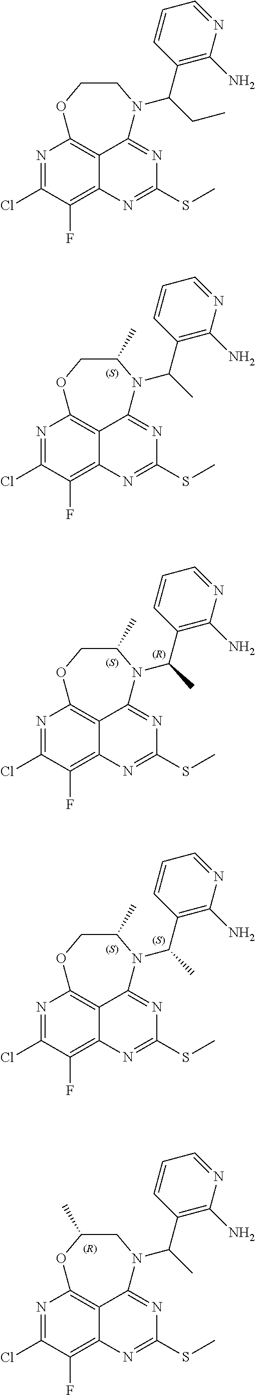

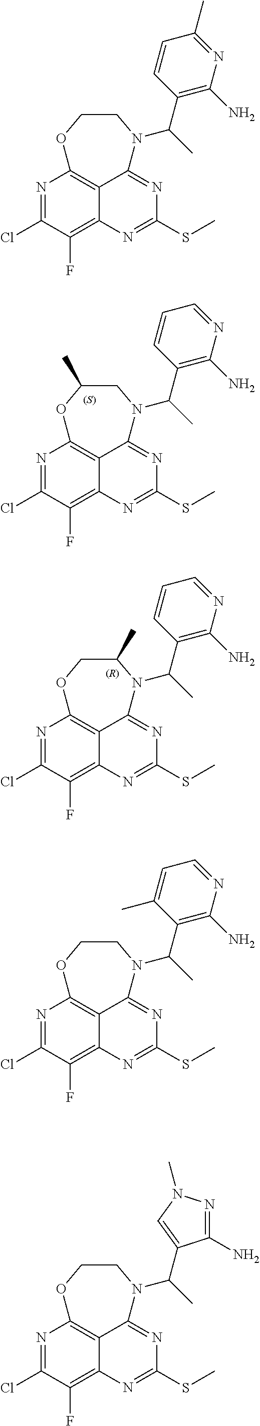

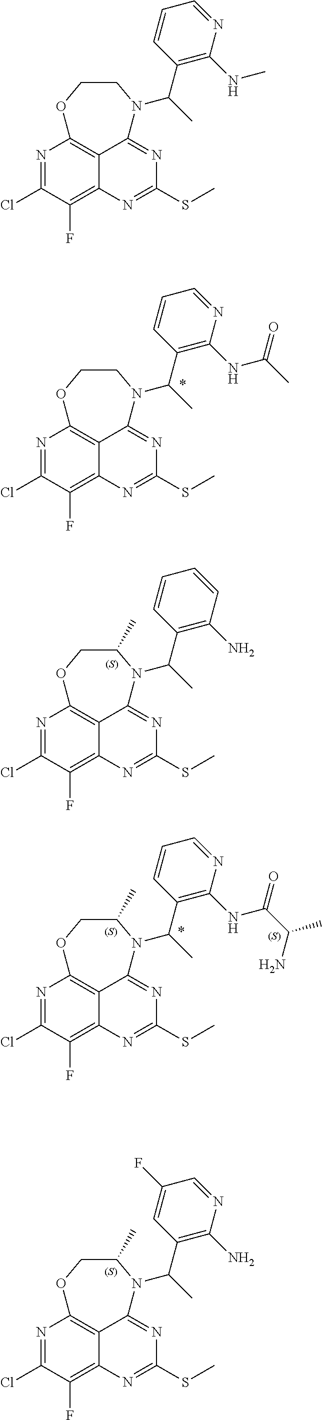

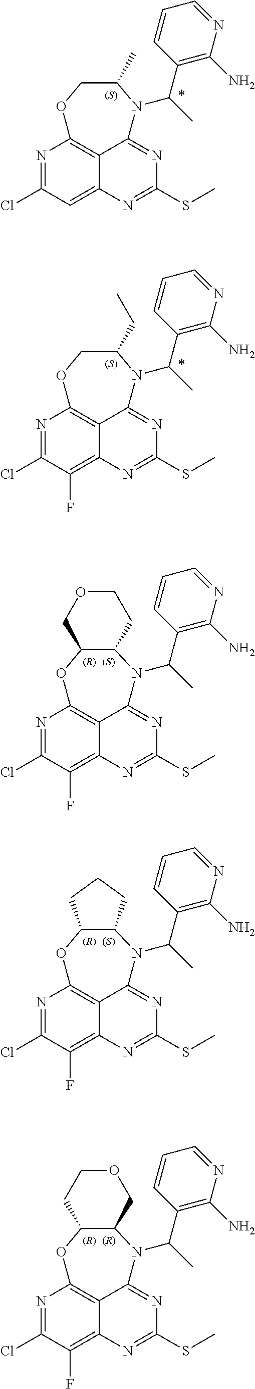

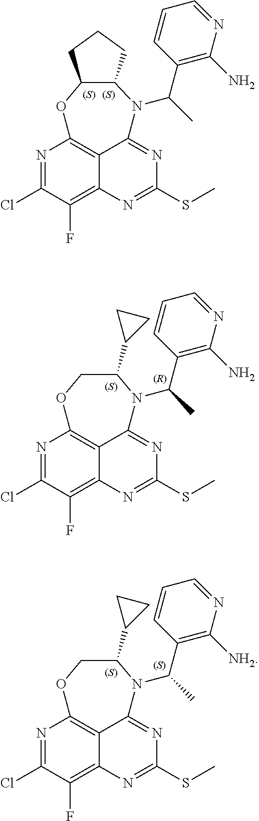

The various embodiments of the present invention (including the following examples) and the features of the various embodiments should be interpreted as being arbitrarily combined with each other, and the various solutions obtained from these mutual combinations are all included in the scope of the present invention, Just like the solutions obtained from the mutual combinations specifically and individually set forth herein, unless clearly stated otherwise in the context.

### Beneficial Effects of the Invention

As mentioned above, it is known that the abnormal activation of RAS proteins is closely related to tumorigenesis, among which KRAS mutations are the most common, and KRAS gene amplification has also been found in a variety of tumors. Inhibiting KRAS mutations or KRAS gene amplification can significantly inhibit cell proliferation in KRAS related diseases, such as cancer. We have surprisingly found that the compounds of the present invention can significantly inhibit KRAS mutations or KRAS gene amplification.

In some embodiments, the compounds of the present invention can inhibit the activity of KRAS in cells, especially the activity of mutant KRAS, for example the activity of one or more mutant KRAS, including but not limited to the activity of KRAS G12C, KRAS G12D, KRAS G12V, KRAS G13D, and/or KRAS Q61H.

In some embodiments, the compounds of the present invention can inhibit cell proliferation in cell lines containing KRAS mutations. For example, the compounds have a cell proliferation inhibitory activity in one or more cell lines containing KRAS mutations, including but not limited to KRAS G12C, KRAS G12D, KRAS G12V, KRAS G13D and/or KRAS Q61H mutations.

In some embodiments, the compounds of the present invention can inhibit cell proliferation in cell lines containing KRAS gene amplification. For example, the compounds have a cell proliferation inhibitory activity in cell lines containing wild-type KRAS gene amplification.

The compounds of the present invention have the aforementioned KRAS inhibitory activity and cell proliferation inhibitory activity, thereby having a potential value as anti-proliferative and/or pro-apoptotic medicaments in the prevention and/or treatment of diseases containing KRAS mutations or KRAS gene amplification, such as cancer or tumor as defined herein.

### General Synthetic Methods

The compound of formula (I) and/or a pharmaceutically acceptable salt thereof described herein can be synthesized using commercially available starting materials, by methods known in the art, or methods disclosed in the present patent application. The synthetic routes shown in Scheme 1 to Scheme 2 illustrate the general synthetic methods of the compounds of the present invention.

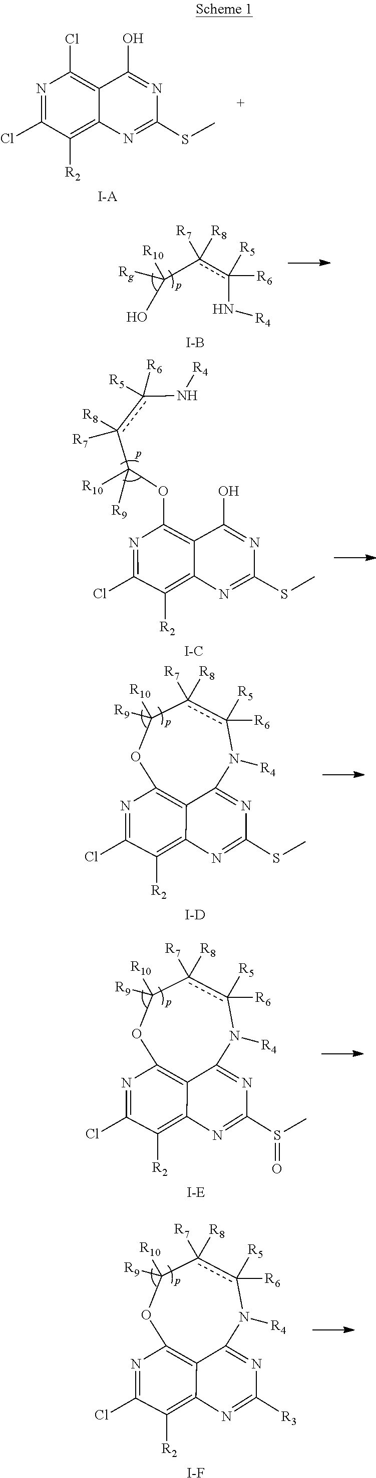

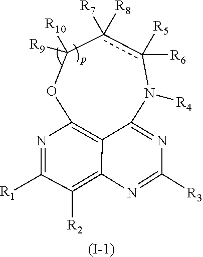

R₁, R₂, R₃, R₄, R₅, R₆, R₇, R₈, R₉, R₁₀ and p are as defined herein.

As shown in Scheme 1, a compound of formula I-A is reacted with a compound of formula I-B under alkaline conditions (such as, but not limited to, sodium hydride) to obtain a compound of formula I-C. The compound of formula I-C is reacted under alkaline conditions (such as, but not limited to, DIEA) in the presence of a condensing agent (such as, but not limited to, BOP-Cl) to obtain a compound of formula I-D. The compound of formula I-D is oxidized in the presence of an oxidant (such as, but not limited to, m-CPBA) to obtain a compound of formula I-E. The compound of formula I-E is reacted with a corresponding alcohol, etc., under alkaline conditions (such as, but not limited to, LiHMDS) to obtain a compound of formula I-F. The compound of formula I-F undergoes a palladium-catalyzed coupling reaction with corresponding borate, boric acid, or alkyl tin etc., under alkaline conditions to obtain a compound of formula (I-1), wherein the alkali used may be selected from Cs₂CO₃, K₂CO₃, K₃PO₄, etc., and the catalyst used may be selected from Pd(dppf)Cl₂·CH₂Cl₂, Pd-G₃, Pd(PPh₃)₄, etc.

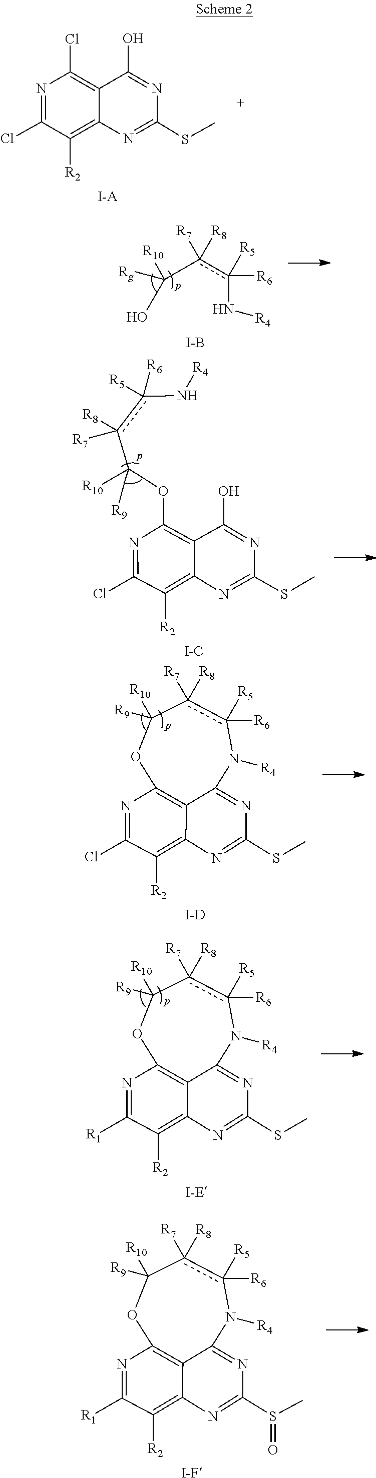

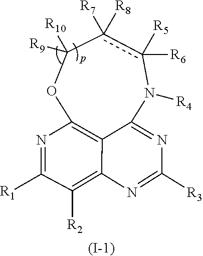

R₁, R₂, R₃, R₄, R₅, R₆, R₇, R₈, R₉, R₁₀ and p are as defined herein.

As shown in Scheme 2, a compound of formula I-A is reacted with a compound of formula I-B under alkaline conditions (such as, but not limited to, sodium hydride) to obtain a compound of formula I-C. The compound of formula I-C is reacted under alkaline conditions (such as, but not limited to, DIEA) in the presence of a condensing agent (such as, but not limited to, BOP-Cl) to obtain a compound of formula I-D. The compound of formula I-D undergoes a palladium-catalyzed coupling reaction with corresponding borate, boric acid, or alkyl tin etc., under alkaline conditions to obtain a compound of formula I-E′, wherein the alkali used may be selected from Cs₂CO₃, K₂CO₃, K₃PO₄, etc., and the catalyst used may be selected from Pd(dppf)Cl₂·CH₂Cl₂, Pd—G₃, Pd(PPh₃)₄, etc. The compound of formula I-E′ is oxidized in the presence of an oxidant (such as, but not limited to, m-CPBA) to obtain a compound of formula I-F′. The compound of formula I-F′ is reacted with a corresponding alcohol, etc., under alkaline conditions (such as, but not limited to, LiHMDS) to obtain a compound of formula (I-1).

The substituents of the compounds thus obtained can be further modified to provide other desired compounds. Synthetic chemistry transformations are described, for example, in R. Larock, Comprehensive Organic Transformations, VCH Publishers (1989); L. Fieser and M. Fieser, Fieser and Fieser's Reagents for Organic Synthesis, John Wiley and Sons (1994); L. Paquette, ed., Encyclopedia of Reagents for Organic Synthesis, John Wiley and Sons (1995) and subsequent editions thereof.

Before use, the compound(s) of the present invention can be purified by column chromatography, high performance liquid chromatography, crystallization or other suitable methods.

### Pharmaceutical Compositions and Utility

The compound of the present invention (e.g., a compound of any of the examples as described herein) is used, alone or in combination with one or more additional therapeutic agents, to formulate pharmaceutical compositions. A pharmaceutical composition comprises: (a) an effective amount of the compounds of the present invention; (b) a pharmaceutically acceptable excipient (e.g., one or more pharmaceutically acceptable carriers); and optionally (c) at least one additional therapeutic agent.

A pharmaceutically acceptable excipient refers to an excipient that is compatible with active ingredients of the composition (and in some embodiments, capable of stabilizing the active ingredients) and not deleterious to the subject to be treated. For example, solubilizing agents, such as cyclodextrins (which form specific, more soluble complexes with the compounds of the present invention), can be utilized as pharmaceutical excipients for delivery of the active ingredients. Examples of other excipients include colloidal silicon dioxide, magnesium stearate, cellulose, sodium lauryl sulfate, and pigments such as D&C Yellow #10. Suitable pharmaceutically acceptable excipients are disclosed in Remington's Pharmaceutical Sciences, A. Osol, a standard reference text in the art.

A pharmaceutical composition comprising a compound of the present invention can be administered in various known manners, such as orally, topically, rectally, parenterally, by inhalation spray, or via an implanted reservoir. The term “parenteral” as used herein includes subcutaneous, intracutaneous, intravenous, intramuscular, intraarticular, intraarterial, intrasynovial, intrasternal, intrathecal, intralesional and intracranial injection or infusion techniques.

A pharmaceutical composition described herein can be prepared in the form of tablet, capsule, sachet, dragee, powder, granule, lozenge, powder for reconstitution, liquid preparation, or suppository. In some embodiments, a pharmaceutical composition comprising a compound of the present invention is formulated for intravenous infusion, topical administration, or oral administration.

An oral composition can be any orally acceptable dosage form including, but not limited to, tablets, capsules, emulsions, and aqueous suspensions, dispersions and solutions. Commonly used carriers for tablets include lactose and corn starch. Lubricating agents, such as magnesium stearate, are also typically added to tablets. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

In some embodiments, the compound of the present invention can be present in an amount of 1, 5, 10, 15, 20, 25, 50, 75, 80, 85, 90, 95, 100, 125, 150, 200, 250, 300, 400 and 500 mg in a tablet. In some embodiments, the compound of the present invention can be present in an amount of 1, 5, 10, 15, 20, 25, 50, 75, 80, 85, 90, 95, 100, 125, 150, 200, 250, 300, 400 and 500 mg in a capsule.

A sterile injectable composition (e.g., aqueous or oleaginous suspension) can be formulated according to techniques known in the art using suitable dispersing or wetting agents (for example, Tween 80) and suspending agents. The sterile injectable composition can also be a sterile injectable solution or suspension in a non-toxic parenterally acceptable diluent or solvent, for example, as a solution in 1,3-butanediol. Among the pharmaceutically acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or di-glycerides). Fatty acids, such as oleic acid and its glyceride derivatives, and natural pharmaceutically acceptable oils, such as olive oil or castor oil, especially in their polyoxyethylated versions, can be used as sterile injectable medium. These oil solutions or suspensions can also contain a long-chain alcohol diluent or dispersant, or carboxymethyl cellulose or similar dispersing agents.

An inhalation composition can be prepared according to techniques well known in the art of pharmaceutical formulation and can be prepared as solutions in saline, employing benzyl alcohol or other suitable preservatives, absorption promoters to enhance bioavailability, fluorocarbons, and/or other solubilizing or dispersing agents known in the art.

A topical composition can be formulated in form of oil, cream, lotion, ointment, and the like. Suitable carriers for the composition include vegetable or mineral oils, white petrolatum (white soft paraffin), branched chain fats or oils, animal fats and high molecular weight alcohols (greater than C12). In some embodiments, the pharmaceutically acceptable carrier is one in which the active ingredient is soluble. Emulsifiers, stabilizers, humectants and antioxidants may also be included as well as agents imparting color or fragrance, if desired. Additionally, transdermal penetration enhancers may be employed in those topical formulations. Examples of such enhancers can be found in U.S. Pat. Nos. 3,989,816 and 4,444,762.

Creams may be formulated from a mixture of mineral oil, self-emulsifying beeswax and water in which mixture the active ingredient, dissolved in a small amount of an oil, such as almond oil, is admixed. An example of such a cream is one which includes, by weight, about 40 parts water, about 20 parts beeswax, about 40 parts mineral oil and about 1 part almond oil. Ointments may be formulated by mixing a solution of the active ingredient in a vegetable oil, such as almond oil, with warm soft paraffin and allowing the mixture to cool. An example of such an ointment is one which includes about 30% by weight almond oil and about 70% by weight white soft paraffin.

The compounds of the present invention are useful as KRAS inhibitors. In some embodiments, the compounds of the present invention are useful as pan-KRAS inhibitors. As used herein, inhibition of the activity of more than one (such as 2, 3, 4, 5 or 6, and the like) mutant KRAS is referred to as pan-KRAS inhibition. In such instances, the compounds of the present invention inhibit the activity of more than one (such as 2, 3, 4, 5 or 6, and the like) mutant KRAS proteins. In some embodiments, the KRAS mutation position corresponds to G12, G13, or Q61. In some embodiments, the KRAS mutation corresponds to G12A, G12C, G12D, G12R, G12S, G12V, G13A, G13C, G13D, G13R, G13S, G13V, Q61E, Q61H, Q61K, Q61L, Q61P, or Q61R mutation. In some embodiments, the KRAS mutation corresponds to G12A, G12C, G12D, G12R, G12S, G12V, G13D, or Q61H mutation. In some embodiments, the KRAS mutation corresponds to G12C, G12D, G12V, G13D, or Q61H mutation.

Suitable in vitro assays can be used to evaluate the effect of the compounds of the present invention in inhibiting the activity of mutant KRAS proteins. The compounds of the present invention can further be examined for additional effects in preventing or treating cancer by in vivo assays. For example, the compound of the present invention can be administered to an animal (e.g., a mouse model) having cancer and its therapeutic effects can be accessed. If the pre-clinical results are successful, the dosage range and administration route for animals, such as humans, can be projected.

The compound of the present invention can be shown to have sufficient pre-clinical practical utility to merit clinical trials hoped to demonstrate a beneficial therapeutic or prophylactic effect, for example, in subjects with cancer.

As used herein, the term “cancer” refers to a cellular disorder characterized by uncontrolled or disregulated cell proliferation, decreased cellular differentiation, inappropriate ability to invade surrounding tissue, and/or ability to establish new growth at ectopic sites. The term “cancer” includes, but is not limited to, solid tumors and hematologic malignancies, such as leukemia, lymphoma or myeloma. The term “cancer” encompasses diseases of skin, tissues, organs, bone, cartilage, blood, and vessels. The term “cancer” further encompasses primary cancer, and metastatic cancer, recurrent cancer and refractory cancer.

Non-limiting examples of solid tumors include pancreatic cancer; bladder cancer; colorectal cancer; colon cancer; rectal cancer; breast cancer, including metastatic breast cancer; prostate cancer, including androgen-dependent and androgen-independent prostate cancer; testicular cancer; renal cancer, including, e.g., metastatic renal cell carcinoma; urothelial carcinoma; liver cancer; hepatocellular cancer; lung cancer, including, e.g., non-small cell lung cancer (NSCLC), small cell lung cancer (SCLC), bronchioloalveolar carcinoma (BAC), and adenocarcinoma of the lung; ovarian cancer, including, e.g., progressive epithelial or primary peritoneal cancer; cervical cancer; endometrial cancer; gastric cancer, including, e.g., chromosomal instability gastric cancer, and gastric adenocarcinoma; esophageal cancer, including, e.g., esophageal adenocarcinoma; head and neck cancer, including, e.g., squamous cell carcinoma of the head and neck; skin cancer, including, e.g., melanoma and basal carcinoma; neuroendocrine cancer, including metastatic neuroendocrine tumors; neuroblastoma; brain tumors, including, e.g., glioma, anaplastic oligodendroglioma, adult glioblastoma multiforme, and adult anaplastic astrocytoma; bone cancer; sarcoma, including, e.g., Kaposi's sarcoma; adrenal carcinoma; mesothelioma; mesothelial carcinoma; choriocarcinoma; muscle carcinoma; connective tissue carcinoma; and thyroid carcinoma.

Non-limiting examples of hematologic malignancies include acute myelogenous leukemia (AML); juvenile acute myelogenous leukemia; chronic myelogenous leukemia (CML), including accelerated phase CML and CML blastic phase (CML-BP); acute lymphocytic leukemia (ALL); B-cell acute lymphocytic leukemia (B-ALL); chronic lymphocytic leukemia (CLL), including high risk CLL; human acute monocytic leukemia (M(5)); hairy cell leukemia; lymphocytic leukemia; chronic lymphoid leukemia; myelogenous leukemia; acute lymphoblastic leukemia; small lymphotic lymphoma (SLL); lymphoblastic lymphoma; Hodgkin's lymphoma; non-Hodgkin's lymphoma (NHL); mantle cell lymphoma (MCL); B-cell lymphoma; T-cell lymphoma; diffuse large B-cell lymphoma (DLBCL); large B-cell lymphoma (LBCL); follicular lymphoma; marginal zone lymphoma; Burkitt's lymphoma; non-Burkitt's highly degree B cell malignant lymphoma; extranodal marginal-zone B-cell lymphoma; multiple myeloma (MM); Waldenstrom's macroglobulinemia; myelodysplastic syndrome (MDS), including refractory anemia (RA), refractory anemia with ring sideroblasts (RARS), refractory anemia with excess of blasts (RAEB) and refractory anemia with excess blasts in transformation (RAEB-T); and myeloproliferative syndrome.

In some embodiments, solid tumor is lung cancer (such as lung adenocarcinoma, non-small cell lung cancer, and small cell lung cancer), colorectal cancer, pancreatic cancer, colon cancer, rectal cancer, thyroid carcinoma, esophageal cancer (such as esophageal adenocarcinoma), cholangiocarcinoma, gallbladder carcinoma, head and neck cancer, breast cancer, endometrial cancer, prostate cancer, ovarian cancer, cervical cancer, neuroblastoma, melanoma, brain tumor, gastric cancer (such as chromosomal instability gastric cancer, and gastric adenocarcinoma), bladder cancer, liver cancer, renal cancer, bone cancer, sarcoma, and adrenal carcinoma. In some embodiments, solid tumor is lung cancer (such as lung adenocarcinoma, non-small cell lung cancer, and small cell lung cancer), colorectal cancer, pancreatic cancer, colon cancer, esophageal cancer (such as esophageal adenocarcinoma), and gastric cancer (such as chromosomal instability gastric cancer and gastric adenocarcinoma).

In some embodiments, hematologic malignancy is acute myelogenous leukemia (AML), juvenile acute myelogenous leukemia, chronic myelogenous leukemia (CML), acute lymphocytic leukemia (ALL), B-cell acute lymphocytic leukemia (B-ALL), acute lymphoblastic leukemia, chronic lymphocytic leukemia (CLL), diffuse large B-cell lymphoma (DLBCL), large B-cell lymphoma (LBCL), B-cell lymphoma, T-cell lymphoma, mantle cell lymphoma, follicular lymphoma, non-Hodgkin's lymphoma, Hodgkin's lymphoma, myelodysplastic syndrome, myeloma (such as multiple myeloma).

The compound of the present invention can be used to achieve a beneficial therapeutic or prophylactic effect, for example, in subjects with cancer.

In addition, the compounds of the present invention (e.g., a compound of any of the examples as described herein) can be administered in combination with additional therapeutic agents for the treatment of diseases or disorders described herein, such as cancer. The additional therapeutic agents may be administered separately with the compound of the present invention or included with such an ingredient in a pharmaceutical composition according to the disclosure, such as a fixed-dose combination drug product. In some embodiments, additional therapeutic agents are those that are known or discovered to be effective in the treatment of diseases containing KRAS mutations or KRAS gene amplification, such as another KRAS inhibitor or a compound active against another target associated with the particular disease. The combination may serve to increase efficacy (e.g., by including in the combination a compound potentiating the potency or effectiveness of the compound of the present invention), decrease one or more side effects, or decrease the required dose of the compound of the present invention.

In some embodiments, the compounds of the present invention (e.g., a compound of any of the examples as described herein) can be administered in combination with additional therapeutic agents, such as anti-neoplastic active agents, anti-inflammatory agents, or immunomodulators, wherein the anti-neoplastic active agents include chemotherapeutic agents, immune checkpoint inhibitors or agonists, and targeted therapeutic agents. The term “anti-neoplastic active agent” as used herein refers to any agent that is administered to a subject suffering from cancer for the purposes of treating the cancer, such as a chemotherapeutic agent, an immune checkpoint inhibitor or agonist, and a targeted therapeutic agent.

Non-limiting examples of chemotherapeutic agents include topoisomerase I inhibitors (e.g., irinotecan, topotecan, camptothecin and analogs or metabolites thereof, and doxorubicin); topoisomerase II inhibitors (e.g., etoposide, teniposide, mitoxantrone, idarubicin, and daunorubicin); alkylating agents (e.g., melphalan, chlorambucil, busulfan, thiotepa, ifosfamide, carmustine, lomustine, semustine, streptozocin, decarbazine, methotrexate, mitomycin C, and cyclophosphamide); DNA intercalators (e.g., cisplatin, oxaliplatin, and carboplatin); free radical generators such as bleomycin; nucleoside mimetics (e.g., 5-fluorouracil, capecitabine, gemcitabine, fludarabine, cytarabine, azacitidine, mercaptopurine, thioguanine, pentostatin, and hydroxyurea); paclitaxel, docetaxel, and related analogs; vincristine, vinblastin, and related analogs; thalidomide and related analogs (e.g., CC-5013 and CC-4047).

Non-limiting examples of immune checkpoint inhibitors or agonists include PD-1 inhibitors, for example, anti-PD-1 antibodies, such as pembrolizumab, nivolumab, and PDR001 (spartalizumab); PD-L1 inhibitors, for example, anti-PD-L1 antibodies, such as atezolizumab, durvalumab, and avelumab; CTLA-4 inhibitors, such as anti-CTLA-4 antibodies, for example ipilimumab; and BTLA inhibitors, LAG-3 inhibitors, TIM3 inhibitors, TIGIT inhibitors, VISTA inhibitors, OX-40 agonists, and the like.

Targeted therapeutic agents include various small molecule or macromolecular targeted therapeutic agents, and non-limiting examples thereof include: protein tyrosine kinase inhibitors (such as imatinib mesylate and gefitinib); proteasome inhibitors (such as bortezomib); NF-κB inhibitors, including IκB kinase inhibitors; EGFR inhibitors; SHP2 inhibitors; IGF1R inhibitors; JAK inhibitors; Met inhibitors; SRC inhibitors; ERK inhibitors; CDK4/6 inhibitors; PI3K inhibitors; SYK inhibitors; Bcl2 inhibitors; IDO inhibitors; A2AR inhibitors; BRAF inhibitors (such as dabrafenib); MEK inhibitors (such as trametinib); mTOR inhibitors (such as rapamycin); anti-CD40 antibodies (such as APX005M, R07009789); antibodies that bind to proteins overexpressed in cancer to down-regulate cell replication, such as anti-CD20 antibodies (such as rituximab, 30 ibritumomab tiuxetan, and tositumomab), anti-Her2 monoclonal antibodies (such as trastuzumab), anti-EGFR antibodies (such as cetuximab) and anti-VEGF antibodies (such as bevacizumab); anti-angiogenic drugs, such as lenalidomide; and other protein or enzyme inhibitors, these proteins or enzymes are known to be upregulated, overexpressed or activated in cancers, and the inhibition of which can down-regulate cell replication.

### EXAMPLES

The examples below are intended to be purely exemplary and should not be considered to be limiting in any way. Efforts have been made to ensure the accuracy with respect to numbers used (for example, amounts, temperature, etc.), but those skilled in the art should understand that some experimental errors and deviations should be accounted for. Unless indicated otherwise, parts are parts by weight, temperature is in degrees Centigrade, and pressure is at or near atmospheric. All MS data were determined by Agilent 6120 or Agilent 1100. All NMR data were generated using a Varian 400 MR machine. All reagents and materials, except synthesized intermediates, used in the present invention are commercially available. All compound names except the reagents are generated by Chemdraw 16.0.

If there is any atom with empty valence(s) in any one of the structures disclosed herein, the empty balance(s) is (are) the hydrogen atom(s) which is (are) omitted for convenience purpose.

In the present application, in the case of inconsistency of the name and structure of a compound, when the two of which are both given for the compound, it is subject to the structure of the compound, unless the context shows that the structure of the compound is incorrect and the name is correct.

### List of Abbreviations Used in the Following Examples

B₂Pin₂ 4,4,4′,4′,5,5,5′,5′-octamethyl-2,2′-bis(1,3,2-dioxaborolane)
(Boc)₂O Di-tert-butyl dicarbonate
BOP-Cl Bis(2-oxo-3-oxazolidinly)phosphinic chloride
cataCXium A Pd—G₃ Methanesulfonato[di(1-adamantyl)-n-butylphosphino](2-amino-1,1′-biphenyl-2-yl)palladium(II)
CD₃OD Deuterated methanol
CDCl₃ Deuterated chloroform
m-CPBA m-Chloroperoxybenzoic acid
DAST Diethylaminosulphur trifluoride
DCM Dichloromethane
DHP 3,4-Dihydropyran
DIEA N,N-diisopropylethylamine
DMA N,N-dimethylacetylamide
DMAP 4-dimethylaminopyridine
DMF N,N-dimethylformamide
DMSO-d₆ Deuterated dimethyl sulfoxide
DPPA Diphenyl azidophosphate
EA Ethyl acetate
Et₃N Triethylamine
EtOH Ethanol
g Gram
(Ir(OMe)(cod))₂ Di-μ-methoxobis(1,5-cyclooctadiene)diiridium(I)
LiHMDS Lithium hexamethyldisilazide
L Liter
LDA Lithium diisopropylamide
M Mole/liter
MeCN Acetonitrile
MeOH Methanol
mg Milligram
mL Milliliter
mmol Millimole
mol Mole
NCS N-chlorosuccinimide
NIS N-iodosuccinimide
NMI N-methylimidazole
PCy₃ Tricyclohexylphosphine
Pd₂(dba)₃ Tris(dibenzylidene acetone)dipalladium
Pd(dppf)Cl₂·CH₂Cl₂ [1,1′-bis(diphenylphosphino)ferrocene]palladium dichloride dichloromethane complex
Pd(PPh₃)₄ Tetra(triphenylphosphine)palladium
Pd(PPh₃)₂Cl₂ Bis(triphenylphosphine)palladium dichloride
PE Petroleum ether
PMB-Cl 4-Methoxybenzylchloride
p-dioxane 1,4-Dioxane
RuPhos Pd G3 Methanesulfonato(2-dicyclohexylphosphino-2′,6′-diisopropoxy-1,1′-biphenyl)(2-amino-1,1′-biphenyl-2-yl)palladium(II)
Select Fluor 1-Chloromethyl-4-fluoro-1,4-diazoniabicyclo[2.2.2]octane bis(tetrafluoroborate)
TCFH Tetramethylchloroformamidinium hexafluorophosphate
TFA Trifluoroacetic acid
THF Tetrahydrofuran
Ti(OEt)₄ Tetraethyl titanate
Tos-Cl p-Toluenesulfonyl chloride

### Example 1 Synthesis of Compounds

### Intermediate I-A1 2-(3-(Ethoxymethoxy)-7,8-difluoronaphthalen-1-yl)-4,4,5,5-tetramethyl-1,3,2-dioxaborolane

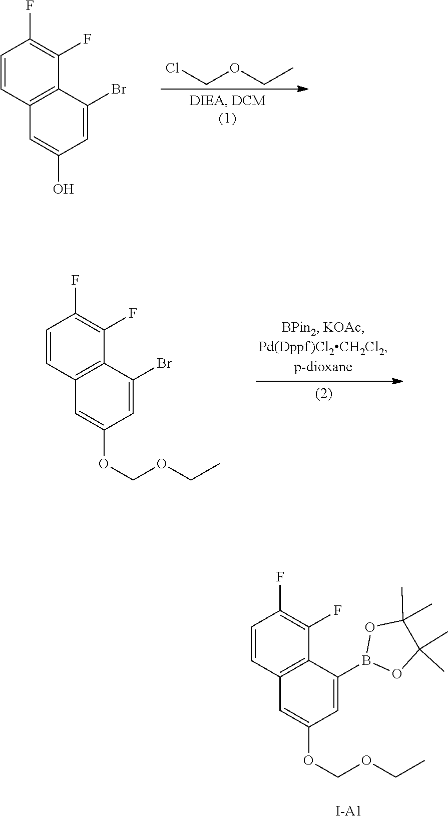

### Step 1: 8-bromo-6-(ethoxymethoxy)-1,2-difluoronaphthalene

4-Bromo-5,6-difluoronaphthalen-2-ol (3.74 g, 14.4 mmol) and DIEA (5.60 g, 43.3 mmol) were dissolved in dichloromethane (50 mL) and cooled to 0° C., and (chloromethoxy)ethane (1.77 g, 18.8 mmol) was added. The reaction solution was stirred at 20° C. for 1 hour and concentrated in vacuum under reduced pressure, and the resulting residue was purified by column chromatography on silica gel (petroleum ether/ethyl acetate) to give the target product (3.43 g, yield 75%) as a purple solid.

### Step 2: 2-(3-(ethoxymethoxy)-7,8-difluoronaphthalen-1-yl)-4,4,5,5-tetramethyl-1,3,2-dioxaborolane

8-Bromo-6-(ethoxymethoxy)-1,2-difluoronaphthalene(3.43 g, 10.8 mmol), B₂Pin₂ (5.49 g, 12.6 mmol), potassium acetate (3.19 g, 32.4 mmol) and Pd(dppf)Cl₂·CH₂Cl₂ (883 mg, 1.08 mmol) were dissolved in 1,4-dioxane (60 mL) and stirred at 70° C. for 5 hours under nitrogen protection. The reaction solution was concentrated in vacuum under reduced pressure, and the resulting residue was purified by column chromatography on silica gel (petroleum ether/ethyl acetate) to give the target product (2.37 g, yield 59%) as a purple oil. [M+H]⁺ 365.2

The intermediates in the table below were prepared from corresponding starting materials and reagents by following the preparation steps of the intermediate I-A1:

| | Structural | LC-MS |
|---|---|---|
| Intermediate | formula | [M + H]⁺ |
| I-A2 | 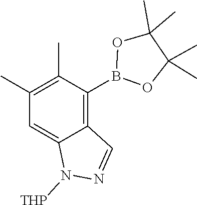 | 357.2 |
| I-A4 | 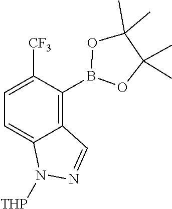 | 397.2 |
| I-A5 | 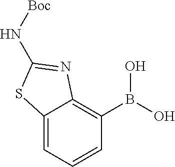 [SMILES: CC(C)(C)OC(=O)Nc1nc2c(B(O)O)cccc2s1] | 295.1 |
| I-A6 | 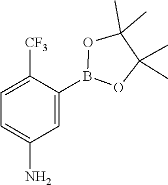 [SMILES: CC1(C)OB(c2cc(N)ccc2C(F)(F)F)OC1(C)C] | 288.1 |
| I-A19 | 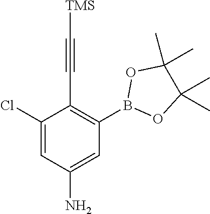 [SMILES: CC1(C)OB(c2cc(N)cc(Cl)c2C#C[Si](C)(C)C)OC1(C)C] | 350.2 |
| I-A22 | 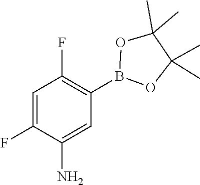 [SMILES: CC1(C)OB(c2cc(N)c(F)cc2F)OC1(C)C] | 256.4 |
| I-A24 | 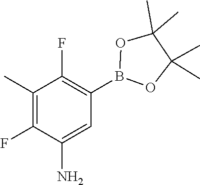 | 270.2 |
| I-A25 | 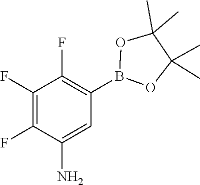 [SMILES: CC1(C)OB(c2cc(N)c(F)c(F)c2F)OC1(C)C] | 274.2 |
| I-A32 | 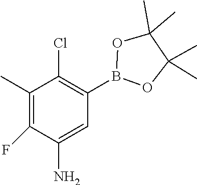 | 286.1 |
| I-A34 | 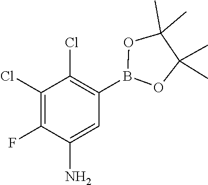 [SMILES: CC1(C)OB(c2cc(N)c(F)c(Cl)c2Cl)OC1(C)C] | 306.1 |
| I-A53 | 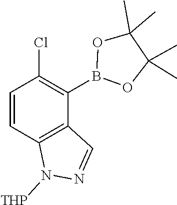 | 363.2 |
| I-A56 | 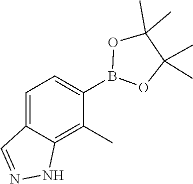 [SMILES: Cc1c(B2OC(C)(C)C(C)(C)O2)ccc2cn[nH]c12] | 259.2 |
| I-A67 | 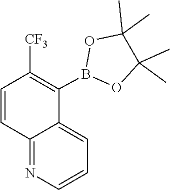 [SMILES: CC1(C)OB(c2c(C(F)(F)F)ccc3ncccc23)OC1(C)C] | 324.2 |
| I-A68 | 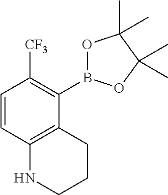 [SMILES: CC1(C)OB(c2c(C(F)(F)F)ccc3c2CCCN3)OC1(C)C] | 328.2 |
| I-A69 | 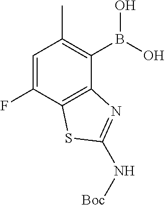 [SMILES: Cc1cc(F)c2sc(NC(=O)OC(C)(C)C)nc2c1B(O)O] | 327.2 |
| I-A70 | 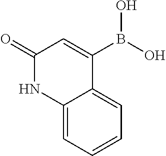 [SMILES: O=c1cc(B(O)O)c2ccccc2[nH]1] | 190.2 |

### Intermediate I-A3

### 2-(3-Chloro-2-cyclopropyl-5-(ethoxymethoxy)phenyl)-4,4,5,5-tetramethyl-1,3,2-dioxaborolane

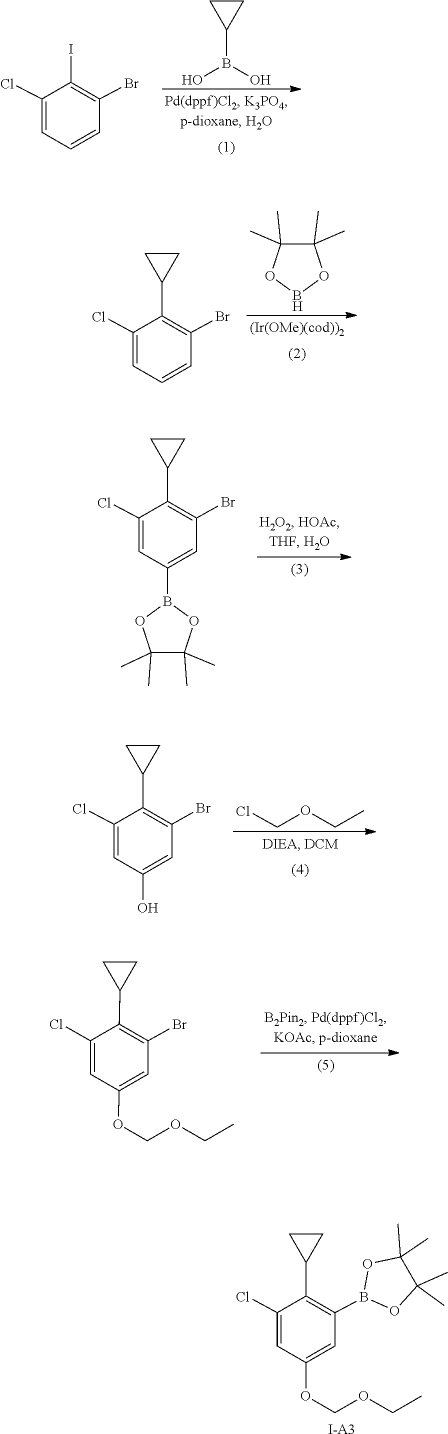

### Step 1: 1-bromo-3-chloro-2-cyclopropylbenzene

1-Bromo-3-chloro-2-iodobenzene (11.0 g, 34 mmol), cyclopropylboronic acid (3.9 g, 45 mmol), potassium phosphate (26.5 g, 125 mmol) and Pd(dppf)Cl₂ (1.3 g, 0.05 mmol) were dissolved in 1,4-dioxane (100 mL) and water (25 mL) and stirred at 100° C. for 18 hours under nitrogen protection. The reaction solution was diluted with water and extracted with ethyl acetate (100 mL×2). The organic phase was collected and concentrated in vacuum under reduced pressure, and the resulting residue was purified by column chromatography on silica gel (petroleum ether/ethyl acetate) to give the target product (6.9 g, yield 86%) as a white oil.

### Step 2: 2-(3-bromo-5-chloro-4-cyclopropylphenyl)-4,4,5,5-tetramethyl-1,3,2-dioxaborolane

1-Bromo-3-chloro-2-cyclopropylbenzene(2.5 g, 10.8 mmol), 4,4,5,5-tetramethyl-1,3,2-dioxaborolane (4.1 g, 32.4 mmol), (Ir(OMe)(cod))₂ (357 mg, 0.5 mmol) and 4-tert-butyl-2-(4-tert-butyl-2-pyridyl)pyridine (173 mg, 0.6 mmol) were dissolved in n-heptane (40 mL) and stirred at 60° C. for 3 hours under nitrogen protection. The reaction solution was concentrated in vacuum under reduced pressure to give a crude target product, which was used in the reaction of the next step directly.

### Step 3: 3-bromo-5-chloro-4-cyclopropylphenol

The crude 2-(3-bromo-5-chloro-4-cyclopropylphenyl)-4,4,5,5-tetramethyl-1,3,2-dioxaborolane was dissolved in tetrahydrofuran (30 mL) and water (15 mL) and cooled to 0° C., and acetic acid (40 mL) and a 30% aqueous hydrogen peroxide solution (106 mL) were added and stirred for 1 hour. The reaction solution was diluted with water (20 mL) and extracted with ethyl acetate (20 mL×2). The organic phase was collected and concentrated in vacuum under reduced pressure, and the resulting residue was purified by reverse phase column chromatography (methanol/water, 0.1% formic acid) to give the target product (1.5 g, two-step yield 50%) as a grey oil. [M−H]⁻ 245.0, 246.9

### Step 4: 1-bromo-3-chloro-2-cyclopropyl-5-(ethoxymethoxy)benzene

3-Bromo-5-chloro-4-cyclopropylphenol (590 mg, 2.4 mmol) and DIEA (925 mg, 7.2 mmol) were dissolved in dichloromethane (6 mL) and cooled to 0° C., and (chloromethoxy)ethane (450 mg, 4.8 mmol) was added. The reaction solution was stirred at 20° C. for 1 hour and concentrated in vacuum under reduced pressure, and the resulting residue was purified by column chromatography on silica gel (petroleum ether/ethyl acetate) to give the target product (670 mg, yield 92%) as a colorless oil.

### Step 5: 2-(3-chloro-2-cyclopropyl-5-(ethoxymethoxy)phenyl)-4,4,5,5-tetramethyl-1,3,2-dioxaborolane

1-Bromo-3-chloro-2-cyclopropyl-5-(ethoxymethoxy)benzene (674 mg, 2.2 mmol), B₂Pin₂ (1.1 g, 4.4 mmol), potassium acetate (650 mg, 6.6 mmol) and Pd(dppf)Cl₂ (925 mg, 0.02 mmol) were dissolved in 1,4-dioxane (100 mL) and stirred at 100° C. for 18 hours under nitrogen protection. The reaction solution was concentrated in vacuum under reduced pressure, and the resulting residue was purified by column chromatography on silica gel (petroleum ether/ethyl acetate) to give the target product (568 mg, yield 73%) as a colorless oil. [M+H]⁺ 353.1

The intermediates in the table below were prepared from corresponding starting materials and reagents by following the preparation steps of the intermediate I-A3:

| Inter- | Structural | LC-MS |
|---|---|---|
| mediate | formula | [M + H]⁺ |
| I-A8 | 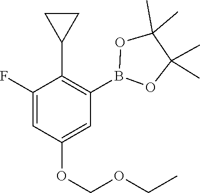 [SMILES: CCOCOc1cc(F)c(C2CC2)c(B2OC(C)(C)C(C)(C)O2)c1] | 337.2 |
| I-A11 | 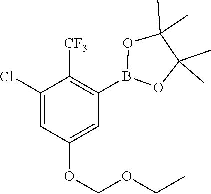 [SMILES: CCOCOc1cc(Cl)c(C(F)(F)F)c(B2OC(C)(C)C(C)(C)O2)c1] | N/A |
| I-A26 | 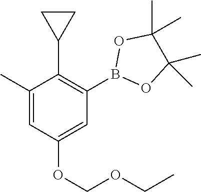 | 333.2 |
| I-A40 | 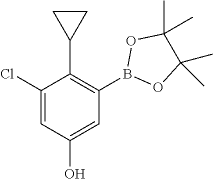 [SMILES: CC1(C)OB(c2cc(O)cc(Cl)c2C2CC2)OC1(C)C] | 293.1 ([M − H]⁻) |
| I-A48 | 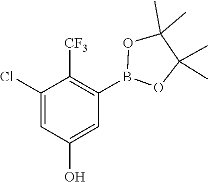 [SMILES: CC1(C)OB(c2cc(O)cc(Cl)c2C(F)(F)F)OC1(C)C] | 321.0 ([M − H]⁻) |
| I-A54 | 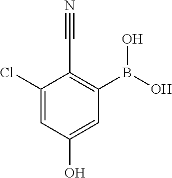 [SMILES: N#Cc1c(Cl)cc(O)cc1B(O)O] | N/A |

### Intermediate I-A9 1-(Tributylstannyl)isoquinolin-3-amine

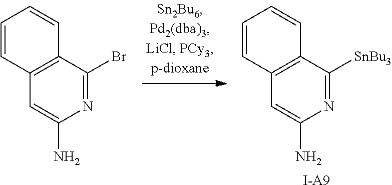

1-Bromoisoquinolin-3-amine (200 mg, 0.9 mmol), hexa-n-butylditin (626 mg, 1.08 mmol), Pd₂(dba)₃ (83 mg, 0.09 mmol), PCy₃ (50 mg, 0.18 mmol) and lithium chloride (190 mg, 4.5 mmol) were added in a 1,4-dioxane solution (5 mL) and stirred at 115° C. for 18 hours under nitrogen protection. After filtration, the cake was washed with ethyl acetate (10 mL). The filtrate was collected and concentrated in vacuum under reduced pressure to give a crude target compound, which was used in the reaction of the next step directly. [M+H]⁺ 435.2

### Intermediate I-A10

### (2-Methoxy-5-(trifluoromethyl)phenyl)boronic acid

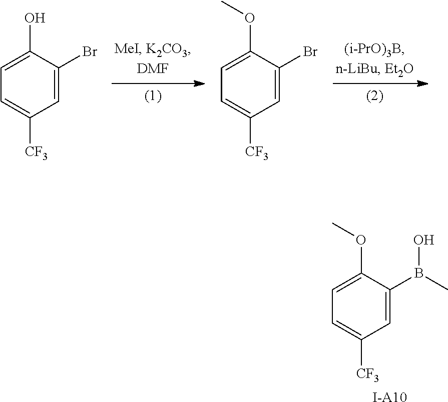

### Step 1: 2-bromo-1-methoxy-4-(trifluoromethyl)benzene

At room temperature, potassium carbonate (3.8 g, 27.5 mmol) and iodomethane (0.7 mL, 11.4 mmol) were added to a solution of 2-bromo-4-(trifluoromethyl)phenol (2 g, 8.3 mmol) in DMF (20 mL) and stirred for 16 hours. The reaction solution was poured into water and extracted with ethyl acetate. The organic phase was collected and concentrated in vacuum under reduced pressure, and the resulting residue was purified by column chromatography on silica gel (petroleum ether/ethyl acetate) to give the target product (1.65 g, yield 78%) as an oil.

### Step 2: (2-methoxy-5-(trifluoromethyl)phenyl)boronic acid

At −70° C., a 2.5 M n-butyl lithium/n-hexane solution (2.9 mL, 7.2 mmol) was added dropwise to a solution of 2-bromo-1-methoxy-4-(trifluoromethyl)benzene (1.65 g, 6.5 mmol) in diethyl ether (25 mL) and stirred for 30 minutes under nitrogen protection. At −70° C., triisopropyl borate (2.4 g, 12.8 mmol) was added dropwise to the reaction solution, gradually heated to room temperature and stirred for 16 hours. A 2 M aqueous hydrochloric acid solution (25 mL) was added to the reaction solution and intensively stirred for 2 hours. The reaction solution was poured into water and extracted with ethyl acetate, the organic phase was collected and concentrated in vacuum under reduced pressure, the resulting residue was slurried in petroleum ether and filtered, and the cake was collected and dried to give the target product (450 mg, yield 32%).

### Intermediate I-A12 3-Methyl-5-(4,4,5,5-tetramethyl-1,3,2-dioxaborolan-2-yl)-4-(trifluoromethyl)aniline

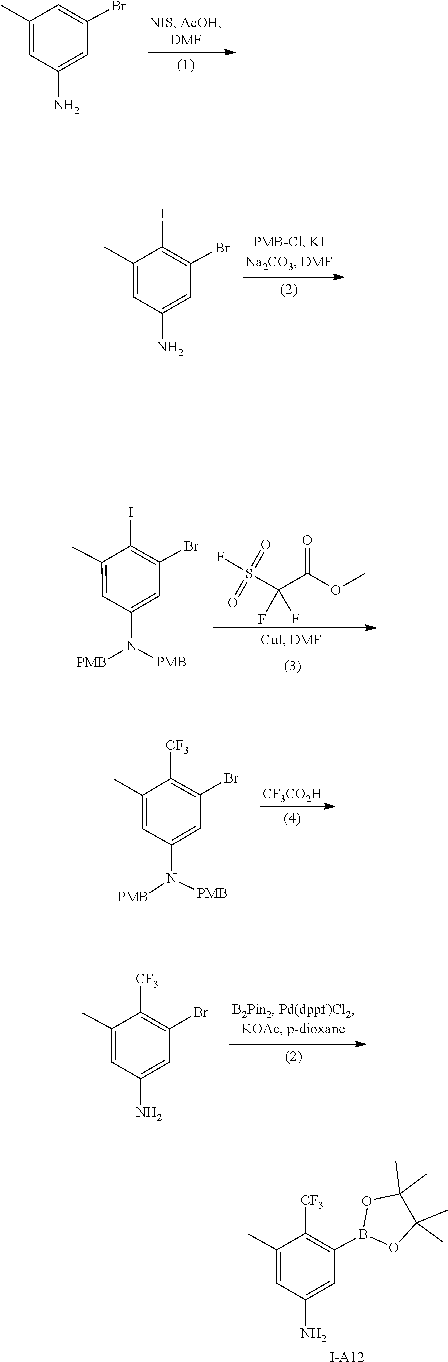

### Step 1: 3-bromo-4-iodo-5-methylaniline

At room temperature, NIS (4.8 g, 21.5 mmol) was added to a solution of 3-bromo-5-methylaniline (4.0 g, 21.5 mmol) in DMF (10 mL) and acetic acid (6 mL) and stirred for 2 hours. Water (10 mL) was added to the reaction solution and extracted with ethyl acetate (40 mL×2), and the organic phase was collected, concentrated in vacuum under reduced pressure and purified by reverse phase column chromatography (acetonitrile/water, 0.1% formic acid) to give the target product (5.4 g, yield 80%) as a yellow solid. [M+H]⁺ 311.9, 313.9

### Step 2: 3-bromo-4-iodo-N,N-bis(4-methoxybenzyl)-5-methylaniline

At room temperature, sodium carbonate (4.6 g, 43.2 mmol), potassium iodide (1.7 g, 10.4 mmol) and PMB-Cl (6.0 g, 38.0 mmol) were added to a solution of 3-bromo-4-iodo-5-methylaniline (5.4 g, 17.3 mmol) in DMF (20 mL). The reaction solution was stirred at 90° C. for 6 hours, cooled to room temperature, quenched with water (10 mL) and extracted with ethyl acetate (50 mL×2), and the organic phase was collected and dried with anhydrous sodium sulfate and filtered. The filtrate was concentrated in vacuum under reduced pressure, and the resulting residue was slurried in ethanol and filtered. The cake was collected and dried to give the target product (7.3 g, yield 77%) as a white solid. [M+H]⁺ 552.0, 554.0

### Step 3: 3-bromo-N,N-bis(4-methoxybenzyl)-5-methyl-4-(trifluoromethyl)aniline

At room temperature, cuprous iodide (13 g, 66.0 mmol) and methyl 2,2-difluoro-2-(fluorosulfonyl)acetate (5.0 g, 23.6 mmol) were added to a solution of 3-bromo-4-iodo-N,N-bis(4-methoxybenzyl)-5-methylaniline (7.3 g, 13.2 mmol) in DMF (15 mL). The reaction solution was stirred at 90° C. for 18 hours and concentrated in vacuum under reduced pressure, and the resulting residue was purified by column chromatography on silica gel (petroleum ether/ethyl acetate) to give the target product (5.1 g, yield 78%) as a yellow solid. [M+H]⁺ 494.0, 496.0

### Step 4: 3-bromo-5-methyl-4-(trifluoromethyl)aniline

3-Bromo-N,N-bis(4-methoxybenzyl)-5-methyl-4-(trifluoromethyl)aniline (5.1 g, 13.2 mmol) was dissolved in trifluoroacetic acid (20 mL). The reaction solution was stirred at 50° C. for 2 hours and concentrated in vacuum under reduced pressure. The resulting residue was diluted with water, adjusted by adding sodium bicarbonate to pH=7 and extracted with ethyl acetate (50 mL×2). The organic phase was collected and concentrated in vacuum under reduced pressure, and the resulting residue was purified by reverse phase column chromatography (acetonitrile/water) to give the target product (1.3 g, yield 50%) as a yellow solid. [M+H]⁺ 254.0, 256.0

### Step 5: 3-methyl-5-(4,4,5,5-tetramethyl-1,3,2-dioxaborolan-2-yl)-4-(trifluoromethyl)aniline

3-Bromo-5-methyl-4-(trifluoromethyl)aniline (1.3 g, 5.1 mmol), B₂Pin₂ (1.1 g, 12.8 mmol), potassium acetate (1.5 g, 15.3 mmol) and Pd(dppf)Cl₂ (372 mg, 0.05 mmol) were dissolved in 1,4-dioxane (100 mL) and stirred at 100° C. for 18 hours under nitrogen protection. After concentration in vacuum under reduced pressure, the resulting residue was purified by column chromatography on silica gel (petroleum ether/ethyl acetate) to give the target product (718 mg, yield 47%) as a grey oil. [M+H]⁺ 302.2

The intermediates in the table below were prepared from corresponding starting materials and reagents by following the preparation steps of the intermediate I-A12:

| Inter- | Structural | LC-MS |
|---|---|---|
| mediate | formula | [M + H]⁺ |
| I-A14 | 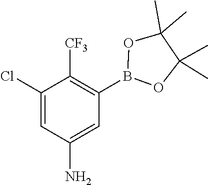 [SMILES: CC1(C)OB(c2cc(N)cc(Cl)c2C(F)(F)F)OC1(C)C] | 322.1 |
| I-A16 | 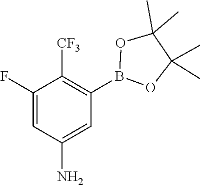 [SMILES: CC1(C)OB(c2cc(N)cc(F)c2C(F)(F)F)OC1(C)C] | 306.2 |
| I-A17 | 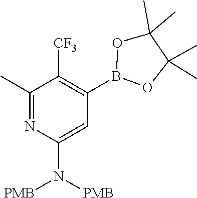 | 543.3 |
| I-A18 | 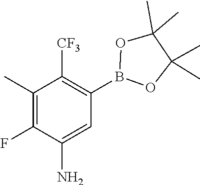 | 320.2 |
| I-A20 | 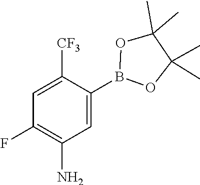 [SMILES: CC1(C)OB(c2cc(N)c(F)cc2C(F)(F)F)OC1(C)C] | 306.2 |
| I-A21 | 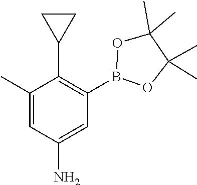 | 274.4 |
| I-A23 | 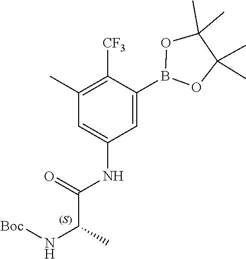 | 473.2 |
| I-A27 | 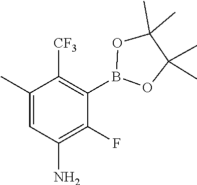 | 320.2 |
| I-A28 | 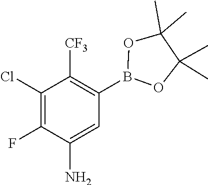 [SMILES: CC1(C)OB(c2cc(N)c(F)c(Cl)c2C(F)(F)F)OC1(C)C] | 340.1 |
| I-A29 | 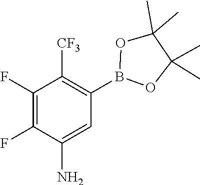 [SMILES: CC1(C)OB(c2cc(N)c(F)c(F)c2C(F)(F)F)OC1(C)C] | 324.1 |
| I-A31 | 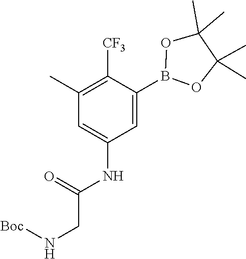 [SMILES: Cc1cc(NC(=O)CNC(=O)OC(C)(C)C)cc(B2OC(C)(C)C(C)(C)O2)c1C(F)(F)F] | 459.1 |
| I-A35 | 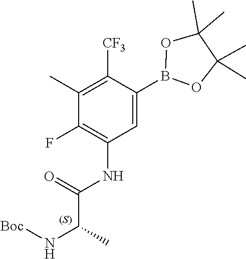 | 491.4 |
| I-A41 | 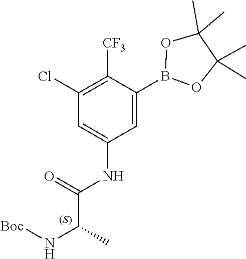 | 393.2 ([M + H − 100]⁺) |
| I-A42 | 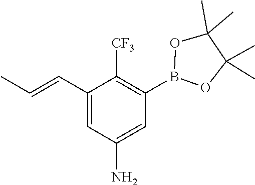 [SMILES: C/C=C/c1cc(N)cc(B2OC(C)(C)C(C)(C)O2)c1C(F)(F)F] | 328.2 |
| I-A45 | 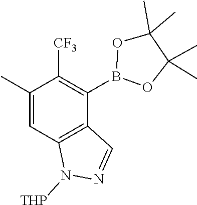 | 411.2 |
| I-A50 | 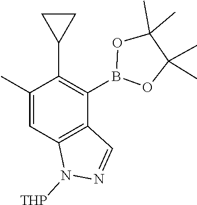 | 383.2 |
| I-A55 | 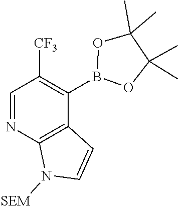 [SMILES: CC1(C)OB(c2c(C(F)(F)F)cnc3c2ccn3COCC[Si](C)(C)C)OC1(C)C] | 443.2 |
| I-A57 | 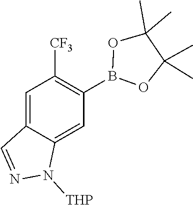 | N/A |
| I-A61 | 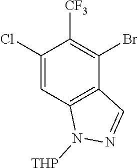 | N/A |
| I-A63 | 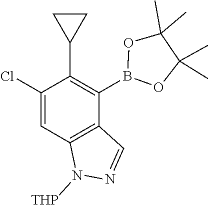 | 403.2 |
| I-A71 | 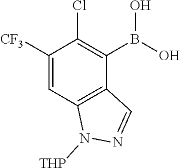 | 349.2 |
| I-A74 | 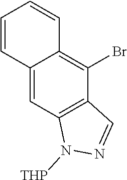 | 331.0, 333.0 |
| I-A84 | 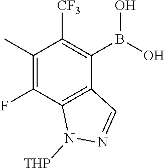 | 347.1 |

### Intermediate I-A13 1-(tetrahydro-2H-pyran-2-yl)-4-(4,4,5,5-tetramethyl-1,3,2-dioxaborolan-2-yl)-5-((trimethylsilyl)ethynyl)-1H-indazole

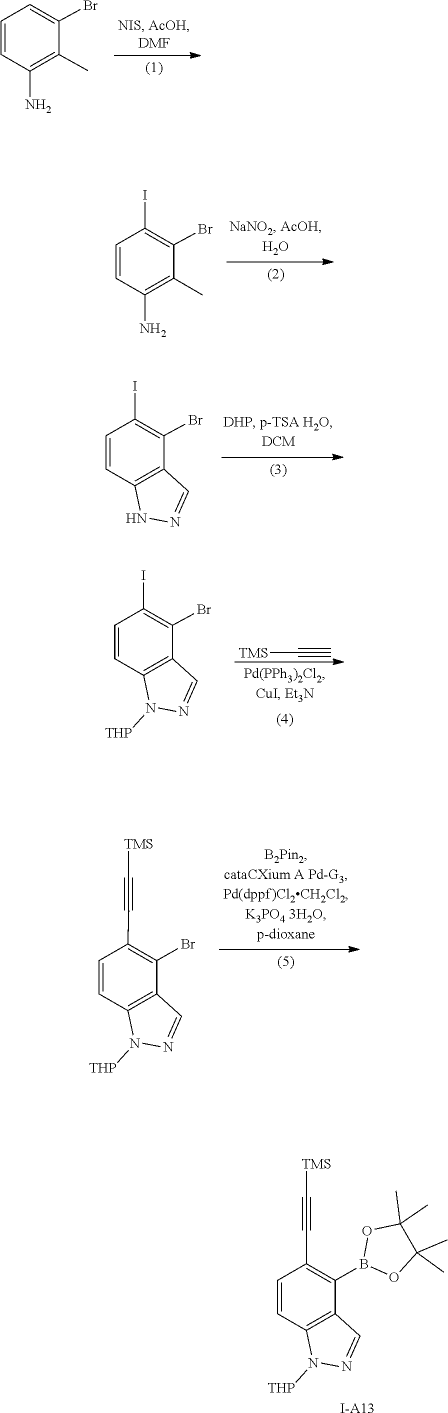

### Step 1: 3-bromo-4-iodo-2-methylaniline

At room temperature, NIS (13.90 g, 61.8 mmol) was added to a solution of 3-bromo-2-methylaniline (11.16 g, 60.0 mmol) in DMF (36 mL) and acetic acid (90 mL) and stirred for 2 hours. After concentration in vacuum under reduced pressure, the resulting residue was dissolved in ethyl acetate and washed with water. The organic phase was collected and concentrated in vacuum under reduced pressure to give a crude target product (18.72 g, yield 100%), which was used in the reaction of the next step directly. [M+H]⁺ 311.9, 313.9

### Step 2: 4-bromo-5-iodo-1H-indazole

At 0-5° C., a solution of sodium nitrite (4.74 g, 71.8 mmol) in water (47 mL) was added dropwise to a solution of 3-bromo-4-iodo-2-methylaniline (18.0 g, 57.6 mmol) in acetic acid (270 mL). The reaction solution was stirred at room temperature for 16 hours and poured into water (1 L). After filtration, the cake was collected and dried to give a crude target product (yield 100%) as a red solid, which was used in the reaction of the next step directly. [M+H]⁺ 322.9, 324.9

### Step 3: 4-bromo-5-iodo-1-(tetrahydro-2H-pyran-2-yl)-1H-indazole

At room temperature, DHP (9.69 g, 115.2 mmol) and p-toluenesulfonic acid monohydrate (1.10 g, 5.76 mmol) were added to a solution of 4-bromo-5-iodo-1H-indazole (57.6 mmol) in dichloromethane (200 mL) and stirred for 2 hours. The reaction solution was washed with an aqueous sodium bicarbonate solution, the organic phase was collected and concentrated in vacuum under reduced pressure, and the resulting residue was purified by column chromatography on silica gel (acetonitrile/water, 0.10% formic acid) to give the target product (12.1 g, yield 52%) as a brown solid. [M+H]⁺ 406.9, 408.9

### Step 4: 4-bromo-1-(tetrahydro-2H-pyran-2-yl)-5-((trimethylsilyl)ethynyl)-1H-indazole

4-Bromo-5-iodo-1-(tetrahydro-2H-pyran-2-yl)-1H-indazole (4.73 g, 11.6 mmol), trimethylsilylacetylene (23.2 mL), Pd(PPh₃)₂Cl₂ (204 mg, 0.29 mmol), cuprous iodide (111 mg, 0.59 mmol) and triethylamine (23.2 mL) were added in tetrahydrofuran (23.2 mL) and stirred at 30° C. for 2 hours under nitrogen protection, and the resulting residue was purified by column chromatography on silica gel (petroleum ether/ethyl acetate) to give the target product (3.30 g, yield 75%) as a brown oil. [M+H]⁺ 377.0, 379.0

### Step 5: 1-(tetrahydro-2H-pyran-2-yl)-4-(4,4,5,5-tetramethyl-1,3,2-dioxaborolan-2-yl)-5-((trimethylsilyl)ethynyl)-1H-indazole

4-Bromo-1-(tetrahydro-2H-pyran-2-yl)-5-((trimethylsilyl)ethynyl)-1H-indazole (3.38 g, 8.94 mmol), B₂Pin₂ (4.54 g, 17.9 mmol), potassium phosphate trihydrate (7.15 g, 26.8 mmol), cataCXium A Pd—G₃ (0.66 g, 0.89 mmol) and Pd(dppf)Cl₂·CH₂Cl₂ (0.73 g, 0.89 mmol) were added in 1,4-dioxane (52 mL) and stirred at 80° C. for 16 hours under nitrogen protection. After concentration in vacuum under reduced pressure, the resulting residue was purified by column chromatography on silica gel (petroleum ether/ethyl acetate) to give the target product (560 mg, yield 15%) as a red oil. [M+H]⁺ 425.3

### Intermediate I-A15 3-Chloro-4-cyclopropyl-5-(4,4,5,5-tetramethyl-1,3,2-dioxaborolan-2-yl)aniline

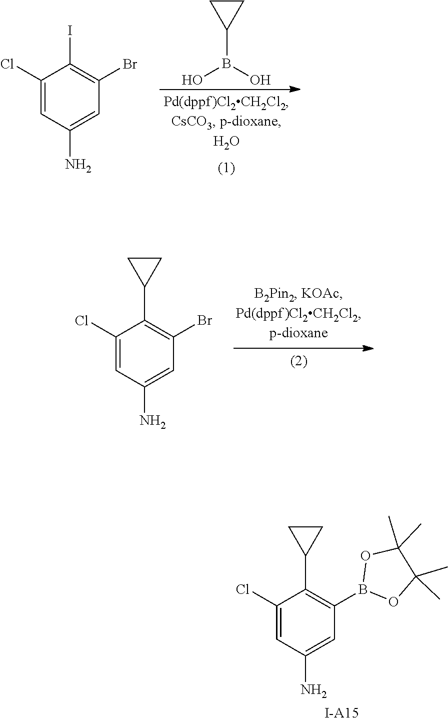

### Step 1: 3-bromo-5-chloro-4-cyclopropylaniline

3-Bromo-5-chloro-4-iodoaniline (1.30 g, 3.9 mmol), cyclopropylboronic acid (605 mg, 7.0 mmol), cesium carbonate (2.55 g, 7.8 mmol) and Pd(dppf)Cl₂·CH₂Cl₂ (319 mg, 0.39 mmol) were dissolved in 1,4-dioxane (50 mL) and water (5 mL) and stirred at 110° C. for 4 hours under nitrogen protection. After concentration in vacuum under reduced pressure, the resulting residue was purified by column chromatography on silica gel (petroleum ether/ethyl acetate) to give the target product (650 mg, yield 67%) as a white solid.

### Step 2: 3-chloro-4-cyclopropyl-5-(4,4,5,5-tetramethyl-1,3,2-dioxaborolan-2-yl)aniline

3-Bromo-5-chloro-4-cyclopropylaniline(450 mg, 1.8 mmol), B₂Pin₂ (558 mg, 2.2 mmol), potassium acetate (539 mg, 5.5 mmol) and Pd(dppf)Cl₂·CH₂Cl₂ (149 mg, 0.18 mmol) were dissolved in 1,4-dioxane (100 mL) and stirred at 90° C. for 18 hours under nitrogen protection. After concentration in vacuum under reduced pressure, the resulting residue was purified by column chromatography on silica gel (petroleum ether/ethyl acetate) to give the target product (280 mg, yield 52%) as a red oil. [M+H]⁺ 294.2

The intermediates in the table below were prepared from corresponding starting materials and reagents by following the preparation steps of the intermediate I-A15:

| | Structural | LC-MS |
|---|---|---|
| Intermediate | formula | [M + H]⁺ |
| I-A33 | 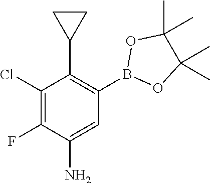 [SMILES: CC1(C)OB(c2cc(N)c(F)c(Cl)c2C2CC2)OC1(C)C] | 312.1 |
| I-A44 | 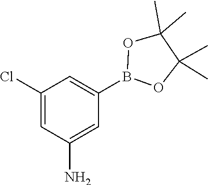 [SMILES: CC1(C)OB(c2cc(N)cc(Cl)c2)OC1(C)C] | 254.2 |

### Intermediate I-A72 5-Fluoro-1-((trifluoromethyl)sulfonyl)-1H-benzo[f]indazol-4-yl trifluoromethanesulfonate

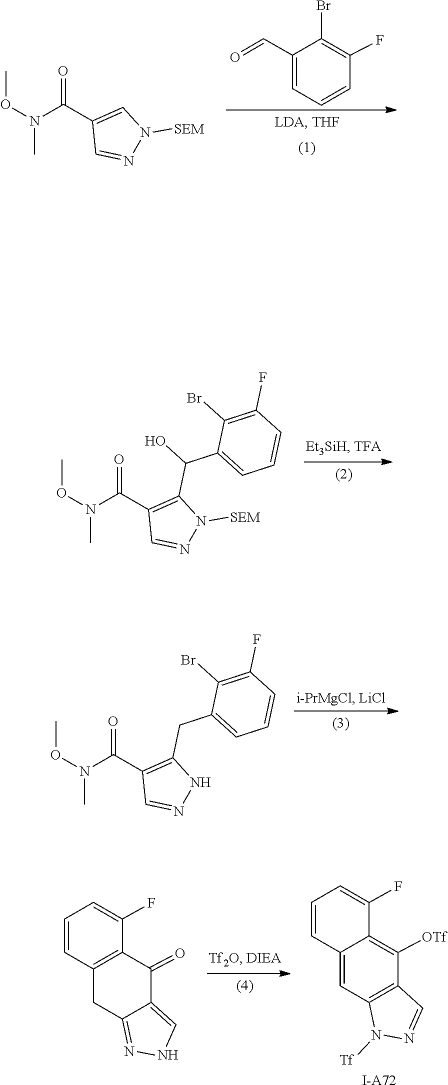

### Step 1: 5((2-bromo-3-fluorophenyl)(hydroxy)methyl)-1-methoxy-N-methyl-1-((2-(trimethylsilyl)ethoxy)methyl)-1H-pyrazole-4-carboxamide

At −78° C. under nitrogen protection, a 2N LDA/tetrahydrofuran solution (10.5 mL, 21.0 mmol) was added dropwise to a solution of N-methoxy-N-methyl-1-((2-(trimethylsilyl)ethoxy)methyl)-1H-pyrazole-4-carboxamide (6.00 g, 21.0 mmol) in tetrahydrofuran (50 mL) and stirred for 30 minutes, and then a solution of 2-bromo-3-fluorobenzaldehyde (6.40 g, 31.5 mmol) in tetrahydrofuran (5 mL) was added dropwise. The reaction solution was stirred at −78° C. for 1 hour, heated to room temperature and stirred overnight, quenched with a saturated aqueous solution of ammonium chloride and extracted with ethyl acetate. The organic phase was collected and concentrated in vacuum under reduced pressure, and the resulting residue was purified by column chromatography on silica gel (petroleum ether/ethyl acetate) to give the target product (6.00 g, yield 58%) as a white solid. [M+H]⁺ 488.2, 490.2

### Step 2: 5-(2-bromo-3-fluorobenzyl)-N-methoxy-N-methyl-1H-pyrazole-4-carboxamide

A solution of 5-((2-bromo-3-fluorophenyl)(hydroxy)methyl)-N-methoxy-N-methyl-1-((2-(trimethylsilyl)ethoxy)methyl)-1H-pyrazole-4-carboxamide (6.00 g, 12.3 mmol), trifluoroacetic acid (20 mL) and triethylsilane (20 mL) in dichloromethane (40 mL) was stirred at 60° C. for 16 hours under nitrogen protection. After concentration in vacuum under reduced pressure, the resulting residue was diluted with ethyl acetate, adjusted with a 2N aqueous sodium hydroxide solution to pH 8, and extracted with ethyl acetate (30 mL×3). The organic phase was collected and combined, concentrated in vacuum under reduced pressure, and purified by column chromatography on silica gel (petroleum ether/ethyl acetate) to give the target product (3.50 g, yield 83%) as a yellow solid. [M+H]⁺ 342.2, 344.2

### Step 3: 5-fluoro-2,9-dihydro-4H-benzo[f]indazol-4-one

At 0° C., a 1.3N isopropylmagnesium chloride lithium chloride/tetrahydrofuran (31.5 mL, 40.9 mmol) solution was added dropwise to a solution of 5-(2-bromo-3-fluorobenzyl)-N-methoxy-N-methyl-1H-pyrazole-4-carboxamide (3.50 g, 10.2 mmol) in tetrahydrofuran (35 mL) and stirred for 2 hours under nitrogen protection. The reaction solution was quenched with 1N hydrochloric acid and adjusted to pH 5, then adjusted with a saturated aqueous solution of sodium bicarbonate to pH 8, and extracted with ethyl acetate (30 mL×3). The organic phase was collected, combined and concentrated in vacuum under reduced pressure, and the resulting residue was washed with ethyl acetate to give the target product (1.44 g, yield 70%) as a yellow solid. [M+H]⁺ 203.2

### Step 4: 5-fluoro-1-((trifluoromethyl)sulfonyl)-1H-benzo[f]indazol-4-yl trifluoromethanesulfonate

At −10° C., trifluoromethanesulfonic anhydride (5.86 g, 20.8 mmol) was added dropwise to a solution of 5-fluoro-2,9-dihydro-4H-benzo[f]indazol-4-one (700 mg, 3.5 mmol) and DIEA (2.68 g, 20.8 mmol) in dichloromethane (10 mL) and stirred for 1 hour under nitrogen protection. The reaction solution was diluted with dichloromethane and washed with a saturated aqueous solution of sodium chloride. The organic phase was collected and concentrated in vacuum under reduced pressure, and the resulting residue was purified by column chromatography on silica gel (petroleum ether/ethyl acetate) to give the target product (1.20 g, yield 74%) as a yellow solid. [M+H]⁺ 467.2

The intermediate in the table below was prepared from corresponding starting materials and reagents by following the preparation steps of the intermediate I-A72:

| | | LC-MS |
|---|---|---|
| Intermediate | Structural formula | [M + H]⁺ |
| I-A76 | 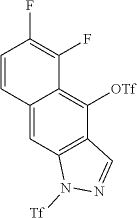 | 485.2 |

### Intermediate I-A73 4-Bromo-7-fluoro-6-methyl-1-(tetrahydro-2H-pyran-2-yl)-5-(trifluoromethyl)-1H-indazole

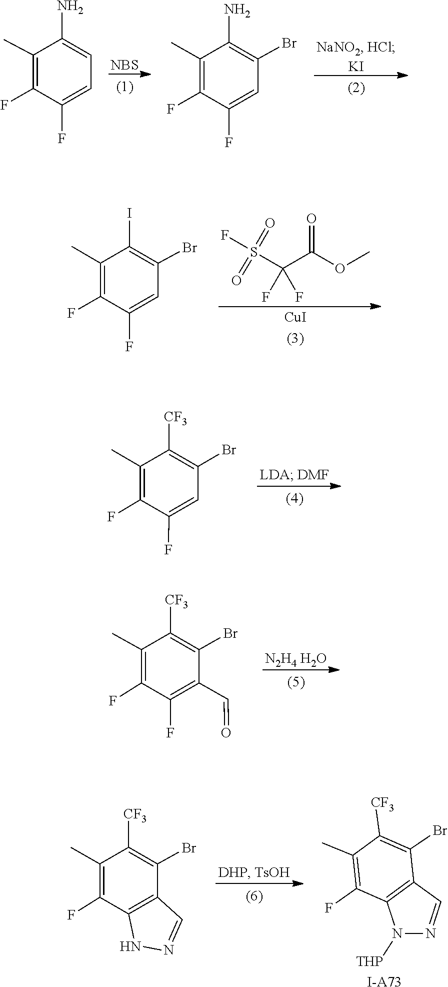

### Step 1: 6-bromo-3,4-difluoro-2-methylaniline

At room temperature, an NBS solid (97.61 g, 548 mmol) was added in batches to a solution of 3,4-difluoro-2-methylaniline (78.5 g, 548 mmol) in DMF (780 mL) and stirred for 30 minutes. The reaction solution was poured into water (2.4 L) and extracted twice with petroleum ether/ethyl acetate (400 mL/400 mL). The organic phase was collected, combined and concentrated in vacuum under reduced pressure to give the target product (121 g, yield 99%) as a red oil, which was used in the reaction of the next step directly.

### Step 2: 1-bromo-4,5-difluoro-2-iodo-3-methylbenzene

At 0° C., 12N hydrochloric acid (65 mL, 780 mmol) was added dropwise to a solution of 6-bromo-3,4-difluoro-2-methylaniline (51.9 g, 234 mmol) in acetonitrile (500 mL). With the internal temperature being maintained at 0° C., a solution of sodium nitrite (19.35 g, 281 mmol) in water (100 mL) was added dropwise and stirred for 30 minutes, and then a solution of potassium iodide (46.6 g, 281 mmol) in water (100 mL) was added dropwise, heated to room temperature and stirred for 3 hours. The resulting solution was adjusted with a 1N aqueous sodium hydroxide solution to pH 8-9, and extracted with ethyl acetate (400 mL×2). The organic phase was collected and combined, washed with an aqueous sodium thiosulfate solution (100 mL) and concentrated in vacuum under reduced pressure, and the resulting residue was purified by column chromatography on silica gel (petroleum ether/ethyl acetate) to give the target product (62.7 g, yield 83%) as a white solid. [M+H]⁺ 294.2

### Step 3: 1-bromo-4,5-difluoro-3-methyl-2-(trifluoromethyl)benzene

A mixture of 1-bromo-4,5-difluoro-2-iodo-3-methylbenzene (62.7 g, 188 mmol), cuprous iodide (305 g, 1.6 mol), methyl 2,2-difluoro-2-(fluorosulfonyl)acetate (307 g, 1.6 mol) and DMF (750 mL) was stirred at 65° C. for 16 hours under nitrogen protection. After filtration, the filtrate was diluted with water (2 L) and extracted with ethyl acetate (600 mL×2). The organic phase was collected and combined, washed with a saturated aqueous solution of sodium chloride and concentrated in vacuum under reduced pressure, and the resulting residue was purified by column chromatography on silica gel (petroleum ether/ethyl acetate) to give the target product (44 g, yield 85%) as a colorless oil.

### Step 4: 2-bromo-5,6-difluoro-4-methyl-3-(trifluoromethyl)benzaldehyde

At −78° C. under nitrogen protection, a 1N LDA/tetrahydrofuran solution (176 mL, 176 mmol) was added dropwise to a solution of 1-bromo-4,5-difluoro-3-methyl-2-(trifluoromethyl)benzene (44 g, 156 mmol) in tetrahydrofuran (200 mL) and stirred for 30 minutes, and then DMF (23.4 g, 320 mmol) was added dropwise and stirred for 2 hours. The reaction solution was poured into an iced saturated aqueous solution of ammonium chloride (1 L) and extracted with ethyl acetate (300 mL×2). The organic phase was collected, combined and concentrated in vacuum under reduced pressure to give the target product (48.5 g, yield 100%) as a yellow oil, which was used in the reaction of the next step directly.

### Step 5: 4-bromo-7-fluoro-6-methyl-5-(trifluoromethyl)-1H-indazole

2-Bromo-5,6-difluoro-4-methyl-3-(trifluoromethyl)benzaldehyde (48.5 g, 156 mmol) was dissolved in tetrahydrofuran (200 mL), and 98% hydrazine hydrate (24.03 g, 50.06 mmol) was added and stirred at 60° C. for 2 hours. The reaction solution was diluted with ethyl acetate (300 mL) and washed with a saturated aqueous solution of sodium chloride. The organic phase was collected and dried with anhydrous sodium sulfate. After filtration, the filtrate was concentrated in vacuum under reduced pressure, the resulting residue was slurried in dichloromethane (50 mL) and filtered, and the resulting solid was collected and dried to give the target product (32.7 g, yield 69%) as a yellow solid. [M+H]⁺ 296.8, 298.8.

### Step 6: 4-bromo-7-fluoro-6-methyl-1-(tetrahydro-2H-pyran-2-yl)-5-(trifluoromethyl)-1H-indazole

At room temperature, p-toluenesulfonic acid monohydrate (2.07 g, 10.9 mmol) was added to a solution of 4-bromo-7-fluoro-6-methyl-5-(trifluoromethyl)-1H-indazole (32.4 g, 109 mmol) and dihydropyran (27.5 g, 327 mmol) in dichloromethane (100 mL) and stirred for 12 hours. The reaction solution was washed with water (50 mL) and extracted with dichloromethane (100 mL×2), and the organic phase was collected, combined and dried with anhydrous sodium sulfate. After filtration, the filtrate was concentrated in vacuum under reduced pressure, and the resulting residue was purified by column chromatography on silica gel (petroleum ether/ethyl acetate) to give the target product (34.3 g, yield 83%) as a white solid. [M+H]⁺ 381.2, 383.2

The intermediates in the table below were prepared from corresponding starting materials and reagents by following the preparation steps of the intermediate I-A73:

| | Structural | LC-MS |
|---|---|---|
| Intermediate | formula | [M + H − 84]⁺ |
| I-A75 | 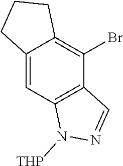 | 237.2, 239.2 |
| | Structural | LC-MS |
| Intermediate | formula | [M + H]+ |
| I-A79 | 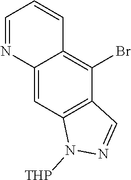 | 331.8, 333.8 |

### Intermediate I-A77 4-Bromo-5,5-difluoro-1-(tetrahydro-2H-pyran-2-yl)-1,5,6,7-tetrahydrocyclopenta[f]indazole

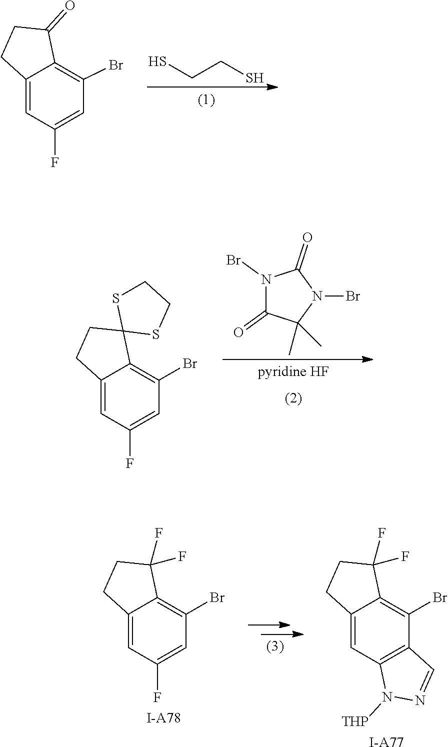

### Step 1: 7-bromo-5-fluoro-2,3-dihydrospiro[indene-1,2′-[1,3]dithiolane]

7-Bromo-5-fluoro-2,3-dihydro-1H-inden-1-one (10.0 g, 43.7 mmol), 1,2-ethanedithiol (4.11 g, 43.7 mmol) and p-toluenesulfonic acid monohydrate (1.66 g, 8.7 mmol) were dissolved in toluene (200 mL) and stirred at 120° C. for 6 hours. The reaction solution was diluted with ethyl acetate and washed with a saturated aqueous solution of sodium chloride, the organic phase was collected and concentrated in vacuum under reduced pressure, and the resulting residue was purified by column chromatography on silica gel (petroleum ether/ethyl acetate) to give the target product (12.8 g, yield 96%) as a white solid.

### Step 2: 7-bromo-1,1,5-trifluoro-2,3-dihydro-1H-indene

At −78° C. under nitrogen protection, pyridine hydrofluoride (20 mL) was added to a mixture of 7-bromo-5-fluoro-2,3-dihydrospiro[indene-1,2′-[1,3]dithiolane](22.5 g, 78.6 mmol) in dichloromethane (100 mL) and stirred for 30 minutes, and then a solution of 1,3-dibromo-5,5-dimethylimidazolidine-2,4-dione (6.0 g, 19.7 mmol) in dichloromethane (100 mL) was added dropwise. The mixture was stirred at −78° C. for 2 hours, heated to room temperature, and then stirred for 3 hours. The reaction solution was poured into a 1N aqueous sodium hydroxide solution (100 mL) and washed with an aqueous sodium bisulfite solution. The organic phase was collected and concentrated in vacuum under reduced pressure, and the resulting residue was purified by column chromatography on silica gel (methanol/water) to give the target product (960 mg, yield 19%) as a colorless solid.

### Step 3: 4-bromo-5,5-difluoro-1-(tetrahydro-2H-pyran-2-yl)-1,5,6,7-tetrahydrocyclopenta[f]indazole

The target product (630 mg, three-step yield 47%) as a colorless oil was prepared from 7-bromo-1,1,5-trifluoro-2,3-dihydro-1H-indene (950 mg) by following the preparation steps 4-6 of the intermediate I-A73. [M+H]⁺ 357.0, 359.2 The intermediate in the table below was prepared from corresponding starting materials and reagents by following the preparation steps of the intermediate I-A77:

| | | LC-MS |
|---|---|---|
| Intermediate | Structural formula | [M + H]⁺ |
| I-A80 | 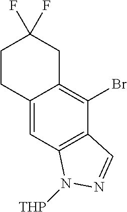 | 371.0, 373.2 |

### Intermediate I-A81 4-Bromo-3,6-dimethyl-1-(tetrahydro-2H-pyran-2-yl)-5-(trifluoromethyl)-1H-indazole

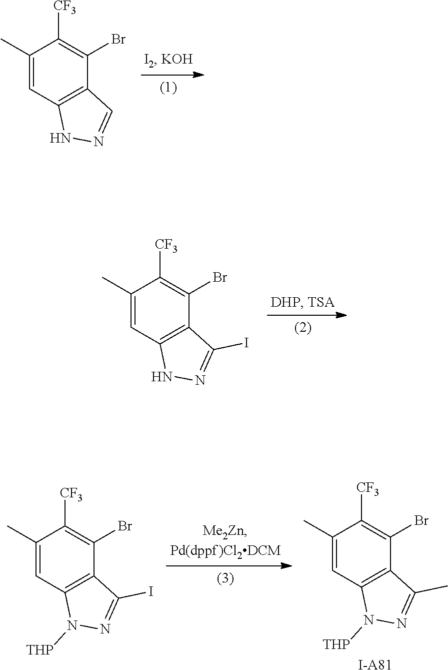

### Step 1: 4-bromo-3-iodo-6-methyl-5-(trifluoromethyl)-1H-indazole

At room temperature, iodine (9.50 g, 37.5 mmol) was added to a solution of 4-bromo-6-methyl-5-(trifluoromethyl)-1H-indazole (4.20 g, 15.0 mmol) and potassium hydroxide (2.50 g, 45.0 mmol) in DMF (20 mL) and stirred for 16 hours. The reaction solution was diluted with ethyl acetate and washed with a saturated aqueous solution of sodium sulfite, the organic phase was collected and concentrated in vacuum under reduced pressure, and the resulting residue was purified by column chromatography on silica gel (petroleum ether/ethyl acetate) to give the target product (4.0 g, yield 66%) as a yellow solid. [M+H]⁺ 404.4, 406.4

### Step 2: 4-bromo-3-iodo-6-methyl-1-(tetrahydro-2H-pyran-2-yl)-5-(trifluoromethyl)-1H-indazole

At room temperature, p-toluenesulfonic acid monohydrate (94 mg, 0.49 mmol) was added to a solution of 4-bromo-3-iodo-6-methyl-5-(trifluoromethyl)-1H-indazole (2.0 g, 4.94 mmol) and dihydropyran (830 mg, 9.88 mmol) in dichloromethane (20 mL) and stirred for 16 hours. The reaction solution was washed with an aqueous sodium bicarbonate solution (20 mL) and extracted with dichloromethane (20 mL×2), and the organic phase was collected, combined and dried with anhydrous sodium sulfate. After filtration, the filtrate was concentrated in vacuum under reduced pressure, and the resulting residue was purified by column chromatography on silica gel (petroleum ether/ethyl acetate) to give the target product (2.0 g, yield 83%) as a white solid.

### Step 3: 4-bromo-3,6-dimethyl-1-(tetrahydro-2H-pyran-2-yl)-5-(trifluoromethyl)-1H-indazole

A 1N dimethylzine/toluene solution (2.2 mL, 2.2 mmol) was added to a solution of 4-bromo-3-iodo-6-methyl-1-(tetrahydro-2H-pyran-2-yl)-5-(trifluoromethyl)-1H-indazole (1.0 g, 2.04 mmol) and Pd(dppf)Cl₂ (165 mg, 0.204 mmol) in 1,4-dioxane (10 mL) under nitrogen protection. The reaction solution was stirred at 100° C. for 1 hour, cooled, diluted with ethyl acetate and washed with an aqueous sodium bicarbonate solution, the organic phase was collected and concentrated in vacuum under reduced pressure, and the resulting residue was purified by column chromatography on silica gel (petroleum ether/ethyl acetate) to give the target product (500 mg, yield 65%) as a yellow solid. [M+H]⁺ 377.0, 379.0

The intermediate in the table below was prepared from corresponding starting materials and reagents by following the preparation steps of the intermediate I-A81:

| | Structural | LC-MS |
|---|---|---|
| Intermediate | formula | [M + H]⁺ |
| I-A82 | 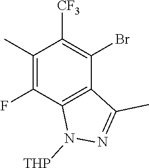 | 310.7, 312.7 [M + H − 84]⁺ |
| I-A86 | 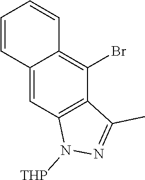 | 344.8, 346.8 |
| I-A87 | 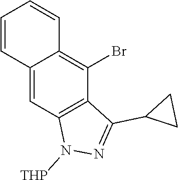 | 370.8, 372.8 |

### Intermediate I-A83 4-Bromo-3-chloro-7-fluoro-6-methyl-1-(tetrahydro-2H-pyran-2-yl)-5-(trifluoromethyl)-1H-indazole

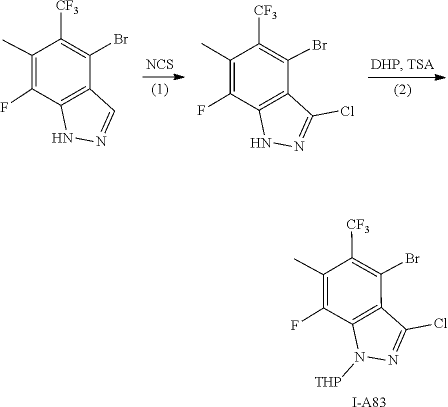

### Step 1: 4-bromo-3-chloro-7-fluoro-6-methyl-5-(trifluoromethyl)-1H-indazole

NCS (279 mg, 2.09 mmol) was added to a solution of 4-bromo-7-fluoro-6-methyl-5-(trifluoromethyl)-1H-indazole (620 mg, 2.09 mmol) in DMF (5 mL) and stirred at 60° C. for 16 hours under nitrogen protection. The reaction solution was poured into water and extracted with ethyl acetate, the organic phase was collected and concentrated in vacuum under reduced pressure, and the resulting residue was purified by column chromatography on silica gel (methanol/water, 0.10% formic acid) to give the target product (470 mg, yield 68%) as a white solid. [M+H]⁺ 332.0

### Step 2: 4-bromo-3-chloro-7-fluoro-6-methyl-1-(tetrahydro-2H-pyran-2-yl)-5-(trifluoromethyl)-1H-indazole

The target product was prepared by following the preparation step 2 of the intermediate I-A81. [M+H]⁺ 416.0

### Intermediate I-B4 (S)-(1-(2,2-difluoroethyl)azetidin-2-yl)methanol

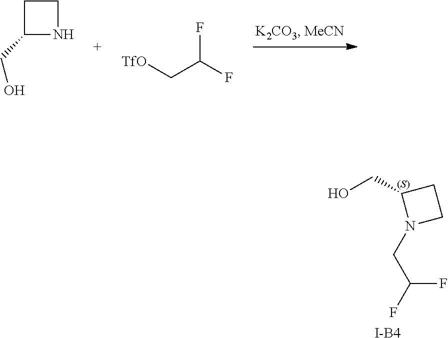

At 0° C., 2,2-difluoroethyl trifluoromethanesulfonate (1.18 g, 5.5 mmol) was added dropwise to a solution of (S)-azetidin-2-ylmethanol (436 mg, 5.0 mmol) and potassium carbonate (1.38 g, 10.0 mmol) in acetonitrile (15 mL). The reaction solution was stirred at room temperature for 5 hours. After filtration, the filtrate was collected and concentrated in vacuum under reduced pressure, and the resulting residue was purified by column chromatography on silica gel (dichloromethane/methanol) to give the target product (420 mg, yield 56%) as a colorless oil. [M+H]⁺ 152.1

### Intermediate I-B10 ((2S,4R)-4-fluoro-1-methylpyrrolidin-2-yl)methanol

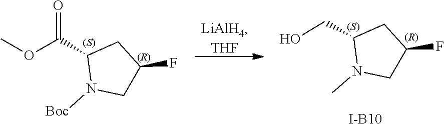

At −10° C., lithium aluminum hydride (461 mg, 12.1 mmol) was added in batches to a solution of 1-(tert-butyl) 2-methyl(2S,4R)-4-fluoropyrrolidine-1,2-dicarboxylate (1.0 g, 4.04 mmol) in tetrahydrofuran (15 mL) and stirred for 30 minutes. The reaction solution was heated to 70° C. and stirred for 3 hours. After cooling to room temperature, a saturated aqueous solution of sodium sulfate was added dropwise, followed by filtration, the filtrate was collected and concentrated in vacuum under reduced pressure, and the resulting residue was purified by column chromatography on silica gel (dichloromethane/methanol) to give the target product (200 mg, yield 38%) as an oil. [M+H]⁺ 134.2. ¹H NMR (400 MHz, CDCl₃) δ 5.20-4.98 (m, 1H), 3.74-3.66 (m, 1H), 3.57-3.30 (m, 2H), 2.86-2.49 (m, 3H), 2.38 (s, 3H), 2.17-1.93 (m, 2H)

### Intermediate I-B11 (R)-(1-(2-fluoroethyl)pyrrolidin-2-yl)methanol

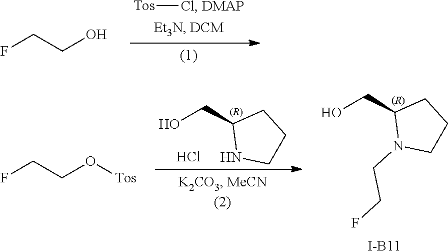

### Step 1: 2-fluoroethyl 4-methylbenzenesulfonate

At 0° C., triethylamine (1.86 mL, 13.3 mmol), a solution of Tos-Cl (2.22 g, 11.7 mmol) in dichloromethane (4 mL), and DMAP (105 mg, 0.86 mmol) were added to a solution of 2-fluoroethanol (500 mg, 7.8 mmol) in dichloromethane (10 mL). The reaction solution was stirred at room temperature for 16 hours, diluted with dichloromethane and washed with a 1 M aqueous hydrochloric acid solution and an aqueous potassium carbonate solution successively, the organic phase was collected and concentrated in vacuum under reduced pressure, and the resulting residue was purified by column chromatography on silica gel (petroleum ether/ethyl acetate) to give the target product (1.5 g, yield 59%) as a yellow oil. [M+H]⁺ 219.1

### Step 2: (R)-(1-(2-fluoroethyl)pyrrolidin-2-yl)methanol

At room temperature, 2-fluoroethyl 4-methylbenzenesulfonate (1.0 g, 4.58 mmol), D-prolinol hydrochloride (628 mg, 4.58 mmol) and potassium carbonate (1.6 g, 11.4 mmol) were added in acetonitrile (10 mL) and stirred for 16 hours. After filtration, the filtrate was collected and concentrated in vacuum under reduced pressure, and the resulting residue was purified by column chromatography on silica gel (dichloromethane/methanol) to give the target product (120 mg, yield 18%) as a yellow oil. [M+H]⁺ 148.4. ¹H NMR (400 MHz, CDCl₃) δ 4.65-4.36 (m, 2H), 3.64-3.58 (m, 1H), 3.44-3.36 (m, 1H), 3.28-3.20 (m, 1H), 3.12-2.97 (m, 1H), 2.75-2.41 (m, 3H), 2.41-2.30 (m, 1H), 1.92-1.65 (m, 4H)

### Intermediate I-B15 (S)-(1-(methyl-d₃)pyrrolidin-2-yl-5,5-d₂)methan-d₂-ol

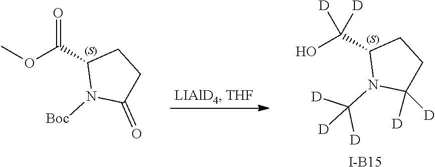

At 0° C., lithium aluminum deuteride (2.61 g, 62.1 mmol) was added in batches to a solution of 1-(tert-butyl)-2-methyl(S)-5-oxopyrrolidine-1,2-dicarboxylate (3.02 g, 12.4 mmol) in tetrahydrofuran (55 mL). The reaction solution was stirred at 70° C. for 3 hours. After cooling to 0° C., water (2.6 mL) was added dropwise, then a 15% aqueous sodium hydroxide solution (2.6 mL) and water (7.8 mL). After filtration, the cake was washed with dichloromethane (100 mL). The filtrate was collected and washed with water, and the organic phase was collected and dried with anhydrous sodium sulfate. After filtration, the filtrate was concentrated under normal pressure to give a crude target product (758 mg, yield 50%) as an oil, which was used in the reaction of the next step directly. [M+H]⁺ 123.2. ¹H NMR (400 MHz, CD₃OD) δ 2.38-2.29 (m, 1H), 2.02-1.91 (m, 1H), 1.78-1.66 (m, 2H), 1.64-1.53 (m, 1H)

The intermediates in the table below were prepared from corresponding starting materials and reagents by following the preparation steps of the intermediate I-B15:

| | Structural | LC-MS |
|---|---|---|
| Intermediate | formula | [M + H]⁺ |
| I-B13 | 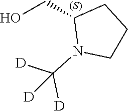 | 119.2 |
| I-B16 | 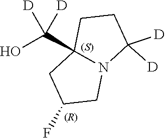 | 164.2 |
| I-B22 | 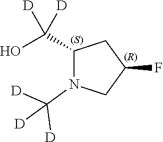 | 139.2 |
| I-B27 | 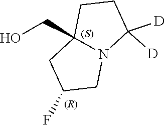 | 162.2 |
| I-B34 | 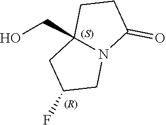 | 174.2 |

### Intermediate I-B18 (S)-(1-(2-tert-butyldimethylsilyl)oxy)ethyl)pyrrolidin-2-yl)methanol

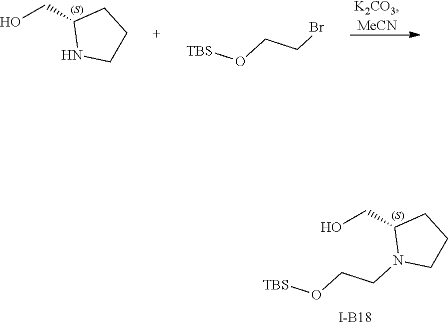

(S)-pyrrolidin-2-ylmethanol (250 mg, 2.47 mmol), (2-bromoethyloxy)(tert-butyl)dimethylsilane (591 mg, 2.47 mmol) and potassium carbonate (681 mg, 4.94 mmol) were added in acetonitrile (15 mL). The reaction solution was stirred at 60° C. for 24 hours. After concentration in vacuum under reduced pressure, the resulting residue was purified by column chromatography on silica gel (water/methanol) to give the target product (450 mg, yield 70%) as a yellow oil. [M+H]⁺ 260.2

### Intermediate I-B23 (S)-(1-(oxetan-3-yl)pyrrolidin-2-yl)methanol

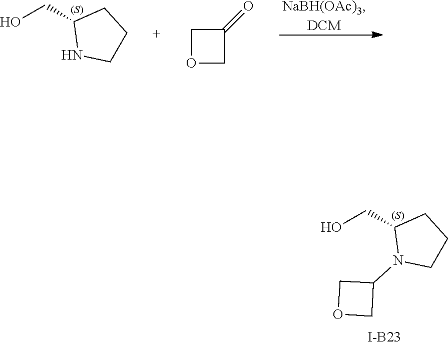

At room temperature, (S)-pyrrolidin-2-ylmethanol (1.62 g, 16.0 mmol) and oxetan-3-one (1.61 g, 22.4 mmol) were added in dichloromethane (64 mL) and stirred for 10 minutes. Sodium triacetoxyborohydride (6.60 g, 32.0 mmol) was added to the reaction solution and stirred at room temperature for 2 hours. After concentration in vacuum under reduced pressure, the resulting residue was purified by column chromatography on silica gel (dichloromethane/methanol) to give the target product (1.79 g, yield 71%) as a yellow oil. [M+H]⁺ 158.2

### Intermediate I-B26 ((2R,7aS)-2-fluorotetrahydro-1H-pyrrolizin-7a(5H)-yl-2,5,5-d₃)methanol

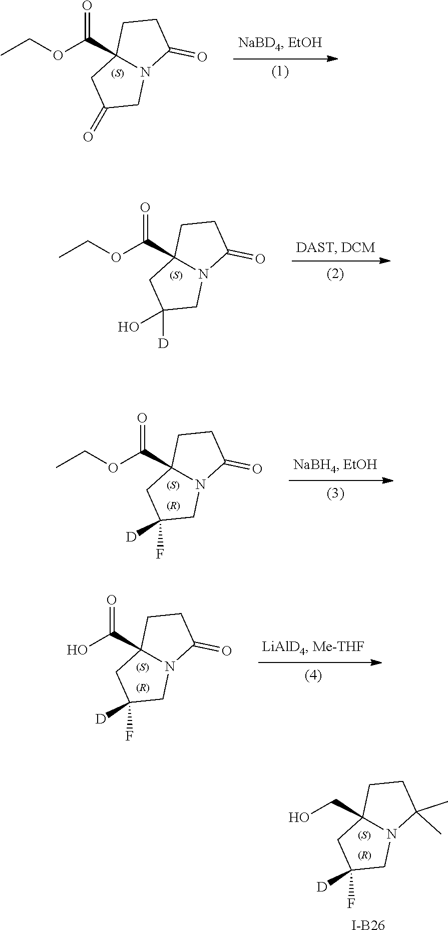

### Step 1: ethyl(7aS)-2-hydroxy-5-oxotetrahydro-1H-pyrrolizine-7a(5H)-carboxylate-2-d

At 0° C., sodium borodeuteride (280 mg, 6.68 mmol) was added in batches to a solution of ethyl(S)-2,5-dioxotetrahydro-1H-pyrrolizine-7a(5H)-carboxylate (4.7 g, 22.3 mmol) in ethanol (50 mL) and stirred for 10 minutes. The reaction solution was quenched with an aqueous ammonium chloride solution and concentrated in vacuum under reduced pressure, and the resulting residue was purified by column chromatography on silica gel (dichloromethane/methanol) to give the target product (3.3 g, yield 69%) as a colorless oil. [M+H]⁺ 215.1

### Step 2: ethyl(2R,7aS)-2-fluoro-5-oxotetrahydro-1H-pyrrolizine-7a(5H)-carboxylate-2-d

At −70° C., DAST (3.3 g, 21 mmol) was added dropwise to a solution of ethyl(7aS)-2-hydroxy-5-oxotetrahydro-1H-pyrrolizine-7a(5H)-carboxylate-2-d (3.0 g, 14.0 mmol) in dichloromethane (50 mL) under nitrogen protection. The reaction solution was heated gradually to room temperature and stirred for 16 hours. The reaction solution was quenched with methanol, and water was added, followed by extraction with dichloromethane. The organic phase was collected and concentrated in vacuum under reduced pressure. The resulting residue was purified by column chromatography on silica gel (petroleum ether/ethyl acetate) to give the target product (800 mg, yield 26%) as an oil. [M+H]⁺ 217.1

### Step 3: (6R,7aS)-6-fluoro-7a-(hydroxymethyl)hexahydro-3H-pyrrolizin-3-one-6-d

At 0° C., sodium borohydride (168 mg, 4.40 mmol) was added to a solution of ethyl(2R,7aS)-2-fluoro-5-oxotetrahydro-1H-pyrrolizine-7a(5H)-carboxylate-2-d (800 mg, 3.68 mmol) in anhydrous ethanol (10 mL). The reaction solution was stirred at room temperature for 1 hour. The reaction solution was quenched with an aqueous ammonium chloride solution and concentrated in vacuum under reduced pressure, and the resulting residue was purified by column chromatography on silica gel (dichloromethane/methanol) to give the target product (650 mg, yield 101%) as an oil. [M+H]⁺ 175.1

### Step 4: ((2R,7aS)-2-fluorotetrahydro-1H-pyrrolizin-7a(5H)-yl-2,5,5-d₃)methanol

The target product was prepared from corresponding intermediates and reagents by following the preparation steps of the intermediate 1-B15. [M+H]⁺ 163.2 The intermediates in the table below were prepared from corresponding starting materials and reagents by following the preparation steps of the intermediate I-B26:

| | Structural | LC-MS |
|---|---|---|
| Intermediate | formula | [M + H]⁺ |
| I-B31 | 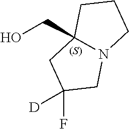 | 161.1 |
| I-B32 | 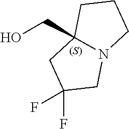 | 178.2 |
| I-B33 | 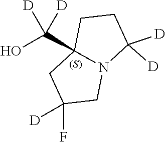 | 165.2 |

### Intermediate I-B40 (1-((cyclopropyl(methyl)amino)methyl)cyclopropyl)methanol

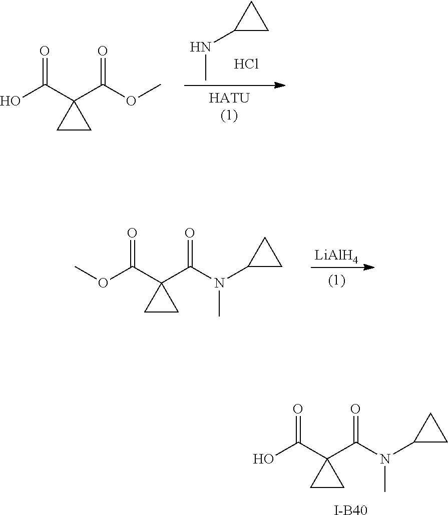

### Step 1: methyl 1-(cyclopropyl(methyl)carbamoyl)cyclopropane-1-carboxylate

At room temperature, DIEA (3.15 g, 24.4 mmol) was added to a solution of 1,1-cyclopropanedicarboxylic acid monomethyl ester (880 mg, 6.1 mmol), N-methylcyclopropanamine (845 mg, 7.9 mmol) and HATU (3.00 g, 7.9 mmol) in DMF (8 mL) and stirred for 1 hour. The reaction solution was purified by column chromatography on silica gel (water/methanol) to give the target product (1.0 g, yield 83%) as a colorless oil. [M+H]⁺ 184.1

### Step 2: (1-((cyclopropyl(methyl)amino)methyl)cyclopropyl)methanol

At 0° C., Lithium Aluminum Hydride (475 mg, 12.5 mmol) was added to a solution of methyl 1-(cyclopropyl(methyl)carbamoyl)cyclopropane-1-carboxylate (1.0 g, 5.07 mmol) in tetrahydrofuran (20 mL). The reaction solution was stirred at 50° C. for 3 hours, cooled to 0° C., quenched with ethyl acetate and poured into water. After filtration, the filter cake was washed with methanol. The filtrate was collected and concentrated, and the residue was purified by column chromatography on silica gel (dichloromethane/methanol) to give the target product (700 mg, yield 89%) as a colorless oil. [M+H]⁺ 156.1

### Intermediate I-C1 5,7-Dichloro-8-fluoro-2-(methylthio)pyrido[4,3-d]pyrimidin-4(3H)-one

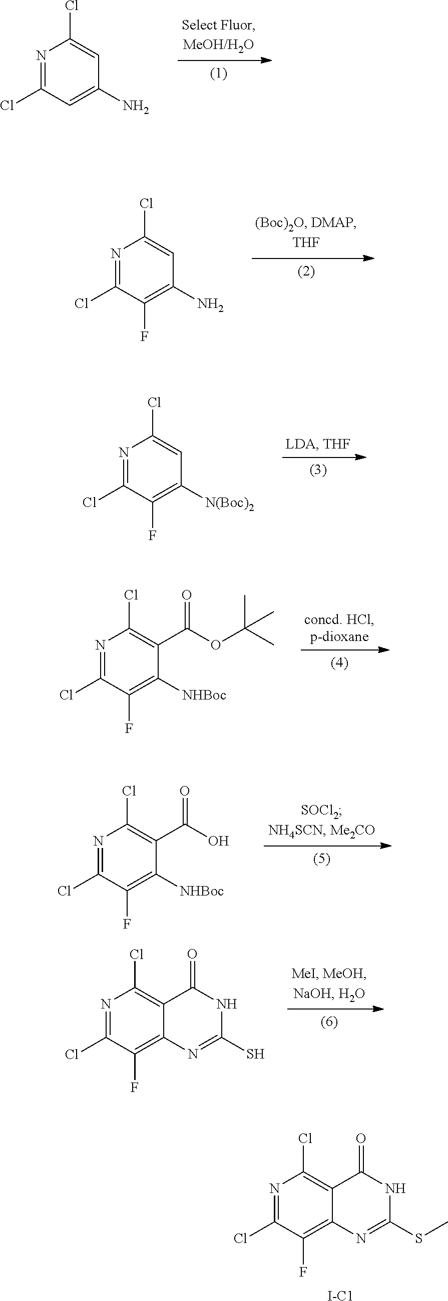

### Step 1: 2,6-dichloro-3-fluoropyridin-4-amine

At room temperature, Select Fluor (146.7 g, 414 mmol) was added to a solution of 2,6-dichloropyridine-4-amine (60.0 g, 368 mmol) in methanol/water (500 mL/100 mL). The reaction solution was stirred at 50° C. for 16 hours, cooled to room temperature and concentrated in vacuum under reduced pressure. The resulting residue was dissolved in ethyl acetate and washed with water, the organic phase was collected and concentrated in vacuum under reduced pressure, and the resulting crude product was purified by column chromatography on silica gel (petroleum ether/ethyl acetate) to give the target product (42.0 g, yield 63%) as a white solid. [M+H]⁺ 181.0, 183.1

### Step 2: tert-butyl (tert-butoxycarbonyl)(2,6-dichloro-3-fluoropyridin-4-yl)carbamate

At room temperature, DMAP (1.42 g, 11.6 mmol) and (Boc)₂O (126.6 g, 580 mmol) were added to a solution of 2,6-dichloro-3-fluoropyridin-4-amine (42.0 g, 232 mmol) in tetrahydrofuran (100 mL). The reaction solution was stirred at 70° C. for 4 hours, cooled to room temperature and concentrated in vacuum under reduced pressure. The resulting residue was slurried in methanol and filtered. The cake was collected and dried to give the target product (73.0 g, yield 83%) as a white solid. [M+H]⁺ 381.1, 383.1

### Step 3: tert-butyl 4-((tert-butoxycarbonyl)amino)-2,6-dichloro-5-fluoronicotinate

At −70° C. under nitrogen protection, a 2.0 M LDA/tetrahydrofuran/n-hexane solution (287 mL, 574 mmol) was added dropwise to a solution of tert-butyl (tert-butoxycarbonyl)(2,6-dichloro-3-fluoropyridin-4-yl)carbamate (73.0 g, 191 mmol) in tetrahydrofuran (500 mL) and stirred at this temperature for 1 hour. The reaction solution was quenched with acetic acid, diluted with ethyl acetate and washed with water. The organic phase was collected and dried with anhydrous sodium sulfate, followed by filtration. The filtrate was concentrated in vacuum under reduced pressure, and the resulting crude product was purified by column chromatography on silica gel (petroleum ether/ethyl acetate) to give the target product (51.0 g, yield 70%) as a white solid. [M+H]⁺ 381.1, 383.1

### Step 4: 4-amino-2,6-dichloro-5-fluoronicotinic acid hydrochloride

At room temperature, concentrated hydrochloric acid (50 mL) was added to a solution of tert-butyl 4-((tert-butoxycarbonyl)amino)-2,6-dichloro-5-fluoronicotinate (51.0 g, 134 mmol) in 1,4-dioxane (200 mL). The reaction solution was stirred at 35° C. for 16 hours and concentrated in vacuum under reduced pressure to give a crude target product (34.9 g, yield 100%), which was used in the reaction of the next step directly. [M+H]⁺ 225.0, 227.0

### Step 5: 5,7-dichloro-8-fluoro-2-mercaptopyrido[4,3-d]pyrimidin-4(3H)-one

At room temperature, crude 4-amino-2,6-dichloro-5-fluoronicotinic acid hydrochloride (34.9 g, 134 mmol) was dissolved in thionyl chloride (300 mL). The reaction solution was stirred at 50° C. for 3 hours, cooled to room temperature and concentrated in vacuum under reduced pressure. The resulting residue was dissolved in acetone (200 mL), and the resulting solution was added dropwise to a solution of ammonium thiocyanate (30.7 g, 403 mmol) in acetone (300 mL) and stirred at room temperature for 1 hour. The reaction solution was diluted with water and extracted with ethyl acetate, and the organic phase was collected and concentrated in vacuum under reduced pressure to give a crude target product (35.0 g, yield 98%), which was used in the reaction of the next step directly. [M+H]⁺ 265.9, 267.9

### Step 6: 5,7-dichloro-8-fluoro-2-(methylthio)pyrido[4,3-d]pyrimidin-4(3H)-one

At room temperature, a solution of sodium hydroxide (10.5 g, 263 mmol) in water (500 mL) and iodomethane (37.4 g, 263 mmol) were added to a solution of crude 5,7-dichloro-8-fluoro-2-mercaptopyrido[4,3-d]pyrimidin-4(3H)-one (35.0 g, 132 mmol) in methanol (500 mL) and stirred for 2 hours. The reaction solution was diluted with water (1 L) and adjusted with concentrated hydrochloric acid to pH=6. After filtration, the cake was washed with water and dried. The resulting solid was slurried in acetonitrile, filtered and dried to give the target product (33.2 g, yield 90%) as a yellow solid. [M+H]⁺ 280.0, 281.9

### Intermediate I-C2 2-((1-(2-Aminopyridin-3-yl)ethyl)amino)ethan-1-ol

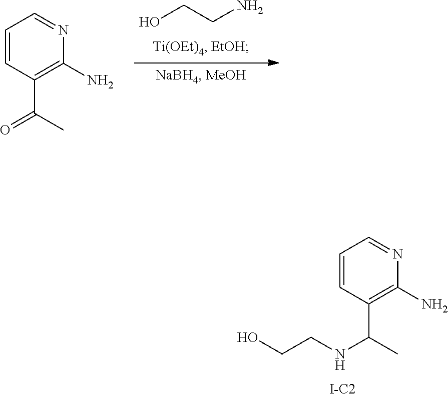

At room temperature, tetraethyl titanate (1 L) was added to a solution of 1-(2-aminopyridin-3-yl)ethan-1-one (100.0 g, 734 mmol) and 2-aminoethan-1-ol (134.6 g, 2.20 mol) in ethanol (1 L). The reaction solution was stirred at 90° C. for 16 hours and cooled to 15-20° C., sodium borohydride (38.9 g, 1.03 mol) was added slowly in batches, and upon completion of the addition, the solution was stirred at room temperature for 2 hours. The reaction solution was poured into water (5 L) and filtered, and the cake was washed with ethanol (0.5 L). The filtrate was collected, combined and concentrated in vacuum under reduced pressure, and the resulting residue was purified by column chromatography on silica gel (dichloromethane/methanol) to give the target product (92.0 g, yield 70%) as a yellow solid. [M+H]⁺ 182.2

### Intermediate I-C3 3-(1-(5-Chloro-4-fluoro-2-(methylthio)-8,9-dihydro-10H-7-oxa-1,3,6,10-tetraazacyclohepta[de]naphthalen-10-yl)ethyl)pyridin-2-amine

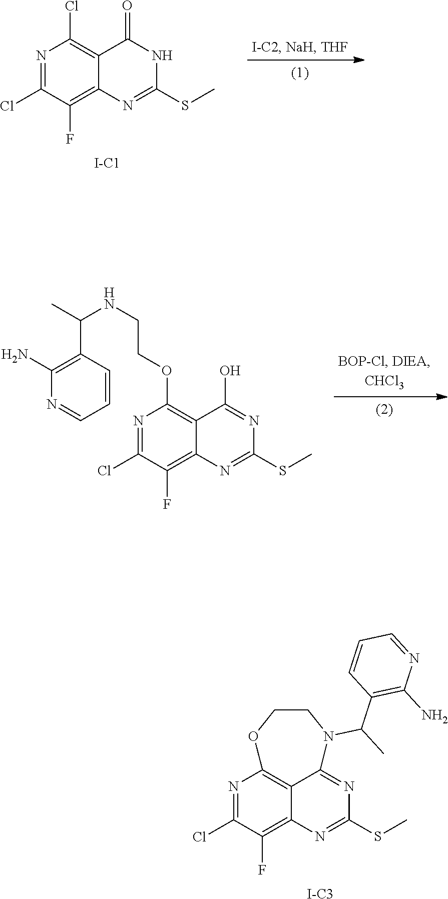

### Step 1: 5-(2-((1-(2-aminopyridin-3-yl)ethyl)amino)ethoxy)-7-chloro-8-fluoro-2-(methylthio)pyrido[4,3-d]pyrimidin-4-ol

At −5-0° C., 60% sodium hydride (2.57 g, 107.1 mmol) was added to a solution of the intermediate I-C2 (7.76 g, 42.8 mmol) in tetrahydrofuran (200 mL) and stirred at 0° C. for 40 minutes. At −5-0° C., the intermediate I-C1 (10.0 g, 35.7 mmol) was added to the reaction solution and stirred at room temperature for 1 hour. The reaction solution was poured into iced water, adjusted with 1.2 M hydrochloric acid to pH=4, and extracted twice with dichloromethane. The aqueous phase was collected and adjusted with a sodium bicarbonate powder to pH=7. After filtration, the cake was collected and dried to give a crude target product (12.0 g, yield 79%) as a yellow solid, which was used in the reaction of the next step directly. [M+H]⁺ 424.1

### Step 2: 3-(1-(5-chloro-4-fluoro-2-(methylthio)-8,9-dihydro-1OH-7-oxa-1,3,6,10-tetraazacyclohepta[de]naphthalen-10-yl)ethyl)pyridin-2-amine

At room temperature, BOP-Cl (8.63 g, 33.9 mmol) and DIEA (10.95 g, 84.7 mmol) were added to a solution of 5-(2-((1-(2-aminopyridin-3-yl)ethyl)amino)ethoxy)-7-chloro-8-fluoro-2-(methylthio)pyrido[4,3-d]pyrimidin-4-ol (12.0 g, 28.2 mmol) in chloroform (250 mL). The reaction solution was stirred at 65° C. for 4 hours and concentrated in vacuum under reduced pressure, the resulting residue was slurried in methanol and filtered, and the cake was collected and dried to give a crude target product (8.8 g, yield 76%) as a yellow solid, which was used in the reaction of the next step directly. [M+H]⁺ 407.1

The intermediates in the table below were prepared from corresponding starting materials and reagents by following the preparation steps of the intermediate I-C3:

| Inter- | Structural | LC-MS |
|---|---|---|
| mediate | formula | [M + H]⁺ |
| I-C10 | 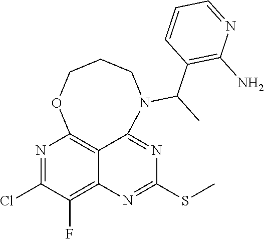 [SMILES: CSc1nc2c3c(nc(Cl)c(F)c3n1)OCCCN2C(C)c1cccnc1N] | 421.1 |
| I-C11 | 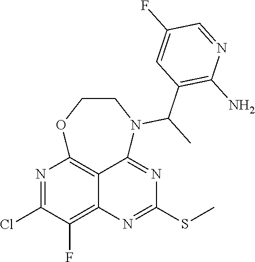 [SMILES: CSc1nc2c3c(nc(Cl)c(F)c3n1)OCCN2C(C)c1cc(F)cnc1N] | 425.2 |
| I-C12 | 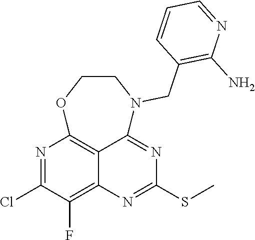 [SMILES: CSc1nc2c3c(nc(Cl)c(F)c3n1)OCCN2Cc1cccnc1N] | 393.1 |
| I-C13 | 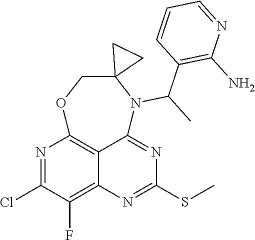 [SMILES: CSc1nc2c3c(nc(Cl)c(F)c3n1)OCC1(CC1)N2C(C)c1cccnc1N] | 433.1 |
| I-C15 | 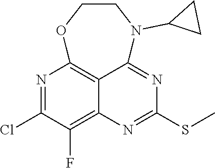 [SMILES: CSc1nc2c3c(nc(Cl)c(F)c3n1)OCCN2C1CC1] | 327.1 |
| I-C16 | 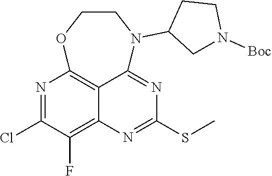 [SMILES: CSc1nc2c3c(nc(Cl)c(F)c3n1)OCCN2C1CCN(C(=O)OC(C)(C)C)C1] | 456.2 |
| I-C18 | 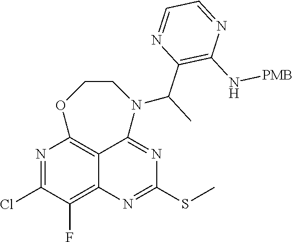 | 528.2 |
| I-C19 | 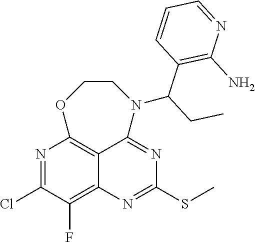 [SMILES: CCC(c1cccnc1N)N1CCOc2nc(Cl)c(F)c3nc(SC)nc1c23] | 421.1 |
| I-C20 | 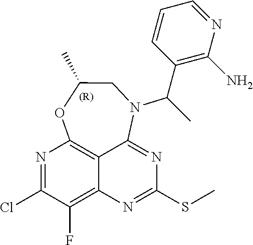 | 421.1 |
| I-C21 | 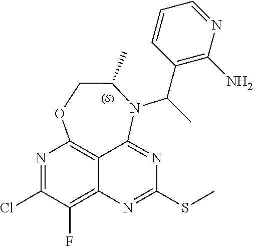 | 421.1 |
| I-C22 | 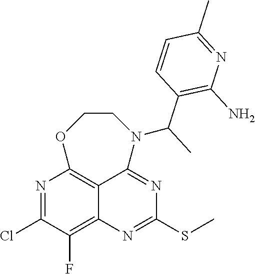 [SMILES: CSc1nc2c3c(nc(Cl)c(F)c3n1)OCCN2C(C)c1ccc(C)nc1N] | 421.1 |
| I-C23 | 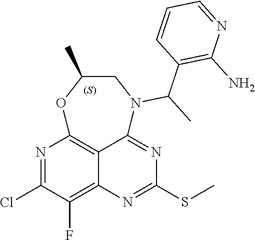 | 421.1 |
| I-C24 | 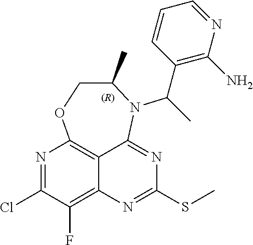 | 421.1 |
| I-C25 | 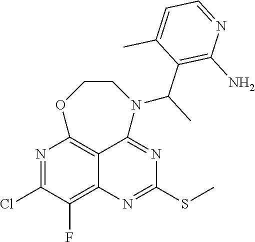 [SMILES: CSc1nc2c3c(nc(Cl)c(F)c3n1)OCCN2C(C)c1c(C)ccnc1N] | 421.1 |
| I-C26 | 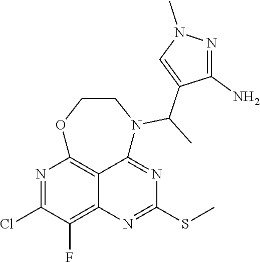 [SMILES: CSc1nc2c3c(nc(Cl)c(F)c3n1)OCCN2C(C)c1cn(C)nc1N] | 410.1 |
| I-C27 | 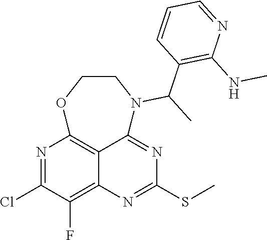 [SMILES: CNc1ncccc1C(C)N1CCOc2nc(Cl)c(F)c3nc(SC)nc1c23] | 421.1 |
| I-C28 | 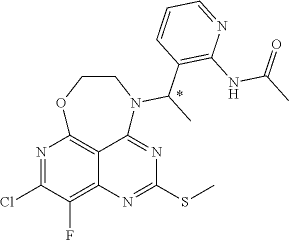 [SMILES: CSc1nc2c3c(nc(Cl)c(F)c3n1)OCCN2C(C)c1cccnc1NC(C)=O] | 449.1 |
| I-C31 | 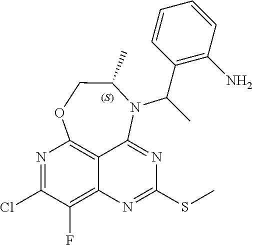 | 420.1 |
| I-C33 | 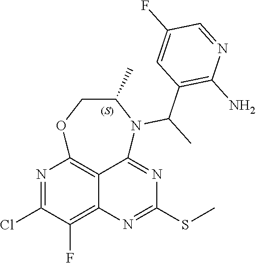 | 439.0 |
| I-C34 | 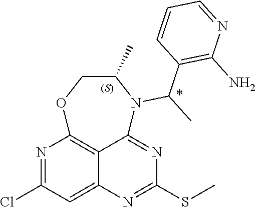 | 403.1 |
| I-C35 | 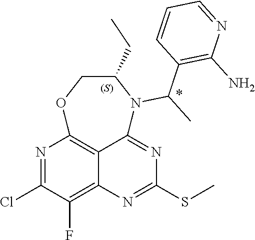 | 435.1 |
| I-C36 | 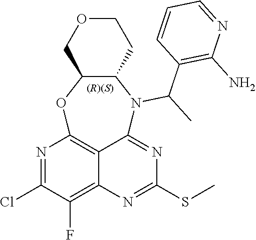 | 463.1 |
| I-C37 | 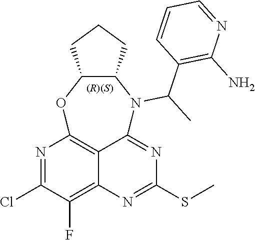 | 447.1 |
| I-C40 | 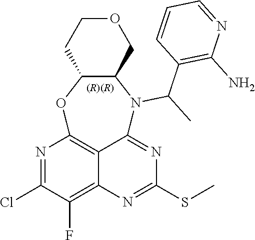 | 463.1 |
| I-C41 | 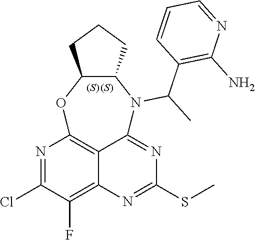 | 447.1 |

### Intermediate I-C4 3-(1-(5-Chloro-4-fluoro-8,9-dihydro-10H-7-oxa-1,3,6,10-tetrazacyclohepta[de]naphthalen-10-yl)ethyl)pyridin-2-amine

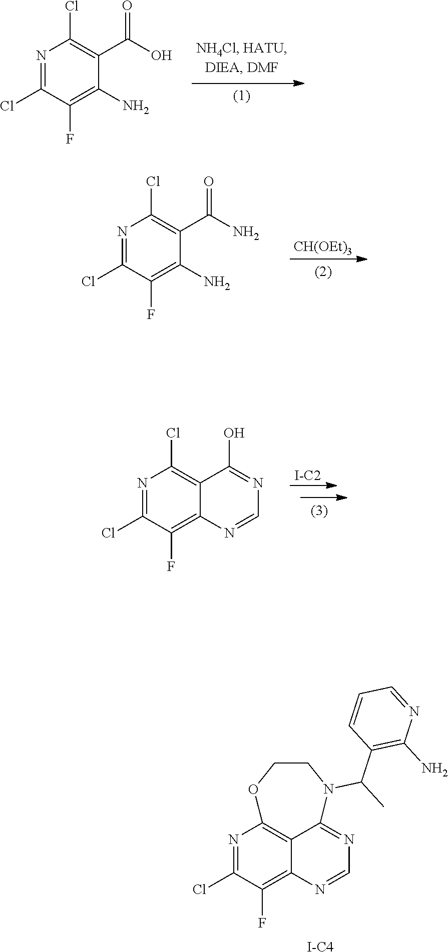

### Step 1: 4-amino-2,6-dichloro-5-fluoronicotinamide

At room temperature, DIEA (1.3 mL, 7.95 mmol), HATU (2.0 g, 5.30 mmol) and ammonium chloride (430 mg, 7.95 mmol) were added to a solution of 4-amino-2,6-dichloro-5-fluoronicotinic acid (700 mg, 2.65 mmol) in DMF (7 mL) and stirred for 2 hours. After concentration in vacuum under reduced pressure, the residue was purified by column chromatography on silica gel (methanol/water) to give the target product (660 mg, yield 1110%) as a white solid. [M+H]⁺ 224.0

### Step 2: 5,7-dichloro-8-fluoropyrido[4,3-d]pyrimidin-4-ol

A mixture of 4-amino-2,6-dichloro-5-fluoronicotinamide (660 mg, 2.95 mmol) and triethyl orthoformate (3 mL) was stirred at 140° C. for 18 hours. After cooling to room temperature, ethyl acetate (20 mL) was added to the reaction solution. After filtration, the cake was washed with ethyl acetate and dried to obtain the target product (370 mg, yield 54%) as a yellow solid. [M+H]⁺ 234.0

### Step 3: 3-(1-(5-chloro-4-fluoro-8,9-dihydro-1OH-7-oxa-1,3,6,10-tetraazacyclohepta[de]naphthalen-10-yl)ethyl)pyridin-2-amine

The target product was prepared from corresponding intermediates and reagents by following the preparation steps of the intermediate I-C3. [M+H]⁺ 361.1 Intermediate I-C5 5-Chloro-4-fluoro-2-(methylthio)-10-(1-(pyridazin-3-yl)ethyl)-9,10-dihydro-8H-7-oxa-1,3,6,10-tetraazacyclohepta[de]naphthalene

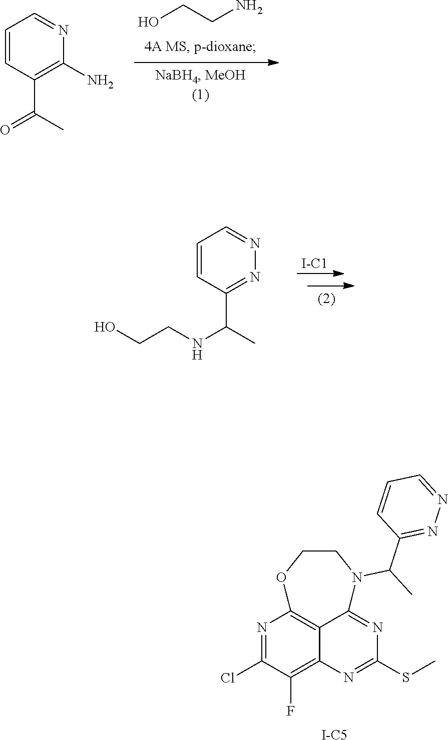

### Step 1: 2-((1-(pyridazin-3-yl)ethyl)amino)ethan-1-ol

At room temperature, 4A molecular sieves (34 g) were added to a solution of 1-(pyridazin-3-yl)ethan-1-one (5.0 g, 41 mmol) and 2-aminoethan-1-ol (7.5 g, 123 mol) in 1,4-dioxane (102 mL). The reaction solution was stirred at 100° C. for 16 hours, cooled to room temperature and filtered, and the filtrate was collected and concentrated in vacuum under reduced pressure. At 0-5° C., sodium borohydride (2.63 g, 69 mmol) was added slowly in batches to a solution of the resulting residue in methanol (102 mL), and upon completion of the addition, the solution was stirred at room temperature for 2 hours. Water (20 mL) was added to the reaction solution and concentrated in vacuum under reduced pressure, and the resulting residue was purified by column chromatography on silica gel (dichloromethane/methanol) to give the target product (5.45 g, yield 79%) as a yellow oil. [M+H]⁺ 168.2

### Step 2: 5-chloro-4-fluoro-2-(methylthio)-10-(1-(pyridazin-3-yl)ethyl)-9,10-dihydro-8H-7-oxa-1,3,6,10-tetraazacyclohepta[de]naphthalene

The target product was prepared from corresponding intermediates and reagents by following the preparation steps of the intermediate I-C3. [M+H]⁺ 391.3

### Intermediate I-C6 3-(1-(5-Chloro-4-fluoro-2-(methylthio)-8,9-dihydro-10H-7-oxa-1,3,6,10-tetraazacyclohepta[de]naphthalen-10-yl-8,8-d₂)ethyl)pyridin-2-amine

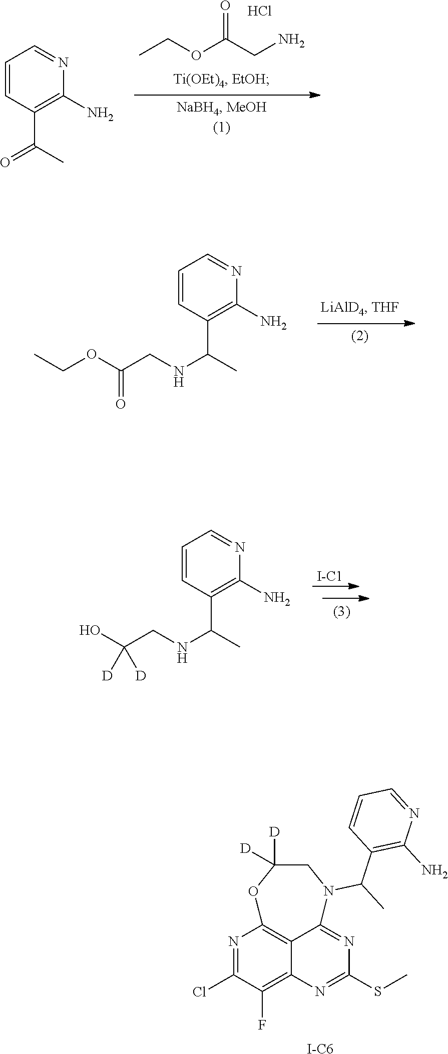

### Step 1: ethyl(1-(2-aminopyridin-3-yl)ethyl)glycinate

At room temperature, tetraethyl titanate (12 mL) was added to a solution of 1-(2-aminopyridin-3-yl)ethan-1-one (2.6 g, 19.0 mmol) and glycine ethylester hydrochloride (4.0 g, 28.6 mmol) in ethanol (12 mL). The reaction solution was stirred at 90° C. for 10 hours and cooled to 15-20° C., sodium borohydride (2.17 g, 57.0 mmol) was added slowly in batches, and upon completion of the addition, the solution was stirred at room temperature for 2 hours. The reaction solution was poured into water (100 mL) and filtered, and the cake was washed with dichloromethane/methanol. The filtrate was collected, combined and concentrated in vacuum under reduced pressure, and the resulting residue was purified by column chromatography on silica gel (dichloromethane/methanol) to give the target product (1.2 g, yield 28%) as a yellow oil. [M+H]⁺ 224.2

### Step 2: 2-((1-(2-aminopyridin-3-yl)ethyl)amino)ethan-1,1-d₂-1-ol

The target product was prepared from lithium aluminum deuteride and corresponding intermediates and reagents by following the preparation steps of the intermediate I-B10. [M+H]⁺ 184.2

### Step 3: 3-(1-(5-chloro-4-fluoro-2-(methylthio)-8,9-dihydro-10H-7-oxa-1,3,6,10-tetraazacyclohepta[de]naphthalen-10-yl-8,8-d₂)ethyl)pyridin-2-amine

The target product was prepared from corresponding intermediates and reagents by following the preparation steps of the intermediate I-C3. [M+H]⁺ 409.1

The intermediates in the table below were prepared from corresponding starting materials and reagents byfollowing thepreparation-steps of the intermediate I-C6:

| | Structural | LC-MS |
|---|---|---|
| Intermediate | formula | [M + H]⁺ |
| I-C7 | 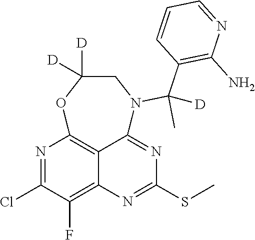 [SMILES: [2H]C1([2H])CN(C([2H])(C)c2cccnc2N)c2nc(SC)nc3c(F)c(Cl)nc(c23)O1] | 410.1 |
| I-C17 | 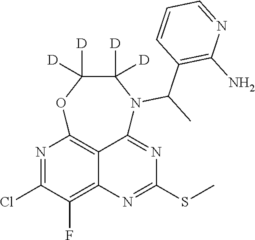 [SMILES: [2H]C1([2H])Oc2nc(Cl)c(F)c3nc(SC)nc(c23)N(C(C)c2cccnc2N)C1([2H])[2H]] | 411.2 |

### Intermediates I-C8 and I-C9 3-(1-(5-Chloro-4-fluoro-2-(methylthio)-8,9-dihydro-10H-7-oxa-1,3,6,10-tetraazacyclohepta[de]naphthalen-10-yl)ethyl)pyridin-2-amine enantiomers

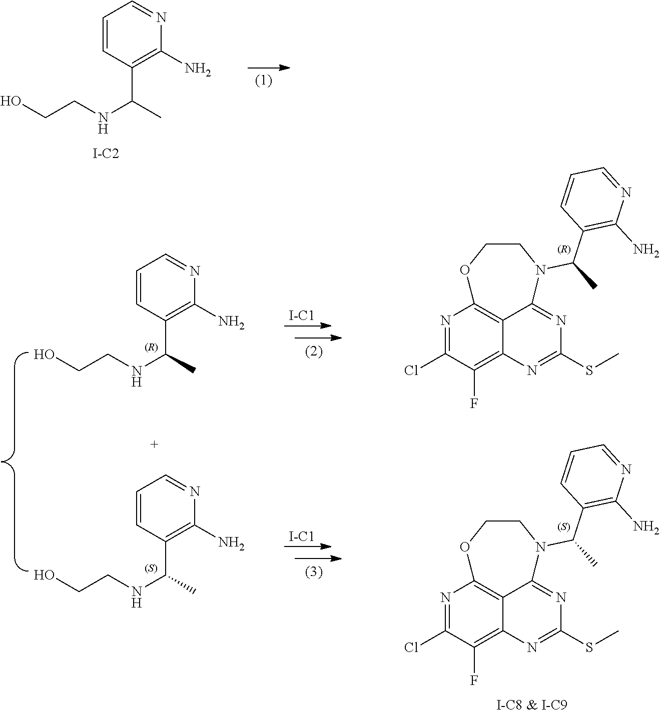

### Step 1: 2-((1-(2-aminopyridin-3-yl)ethyl)amino)ethan-1-ol enantiomers

2-((1-(2-Aminopyridin-3-yl)ethyl)amino)ethan-1-ol (60.0 g) was revolved by chiral HPLC to obtain one pair of enantiomers. Chiral HPLC analysis conditions: column: OD-H (0.46 cm I.D.×15 cm L); mobile phase: carbon dioxide/ethanol (0.05% ethylenediamine)=70:30; flow rate: 2.5 mL/minute; detector: UV 254 nm; first eluate (19.0 g, RT=2.877 minutes), ee %=100%, second eluate (17.5 g, RT=3.124 minutes), ee %=100%.

### Step 2 and step 3: 3-(1-(5-chloro-4-fluoro-2-(methylthio)-8,9-dihydro-10H-7-oxa-1,3,6,10-tetraazacyclohepta[de]naphthalen-10-yl)ethyl)pyridin-2-amine enantiomers

The target intermediates were prepared respectively from corresponding chiral raw materials and reagents by following the preparation steps of the intermediate I-C3. The enantiomers in the table were subjected to chiral HPLC analysis under the following conditions (flow rate: 1 mL/minute; detector: UV 254 nm):

| Intermediate | Column | Mobile phase | RT/minute | ee % |
|---|---|---|---|---|
| I-C8 | ODH | Ethanol (0.1% | 9.414 | 100% |
| I-C9 | (0.46 × 25 cm) | diethylamine) | 12.663 | 100% |

### Intermediate I-C14 3-(1-(5-Chloro-4-fluoro-2-(methylthio)-8,9-dihydro-10H-7-oxa-1,3,6,10-tetraazacyclohepta[de]naphthalen-10-yl)cyclopropyl)-N-(4-methoxybenzyl)pyridin-2-amine

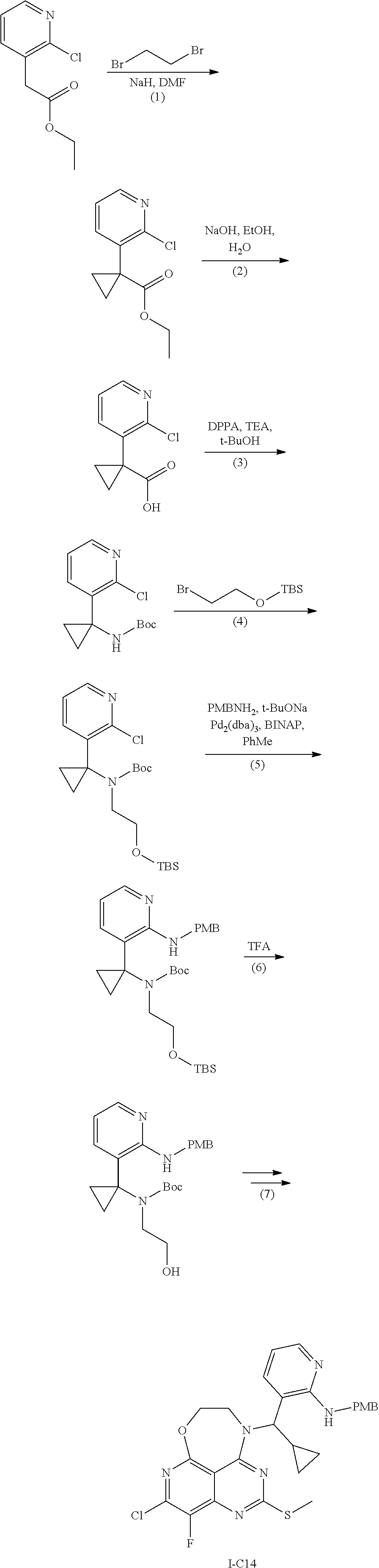

### Step 1: ethyl 1-(2-chloropyridin-3-yl)cyclopropane-1-carboxylate

At −5-0° C., 60% sodium hydride (3.26 g, 81.6 mmol) was added to a solution of ethyl 2-(2-chloropyridin-3-yl)acetate (3.99 g, 20.0 mmol) in DMF (80 mL) and stirred at 0° C. for 1 hour. At −5-0° C., 1,2-dibromoethane (5.65 g, 30.0 mmol) was added to the reaction solution and stirred at 0° C. for 2 hours. The reaction solution was poured into an aqueous ammonium chloride solution and extracted with ethyl acetate. The organic phase was collected, washed with a saturated aqueous solution of sodium chloride and concentrated in vacuum under reduced pressure, and the resulting residue was purified by column chromatography on silica gel (petroleum ether/ethyl acetate) to give the target product (3.60 g, yield 80%) as a yellow oil. [M+H]⁺ 226.1

### Step 2: 1-(2-chloropyridin-3-yl)cyclopropane-1-carboxylic acid

A solution of ethyl 1-(2-chloropyridin-3-yl)cyclopropane-1-carboxylate (3.60 g, 15.9 mmol) and sodium hydroxide (3.18 g, 79.5 mmol) in ethanol (80 mL) and water (40 mL) was stirred at 80° C. for 24 hours. The resulting solution was concentrated in vacuum under reduced pressure and adjusted with 1 M hydrochloric acid to pH 7. After extraction with ethyl acetate, the organic phase was collected and washed with a saturated aqueous solution of sodium chloride. The organic phase was dried with anhydrous sodium sulfate and filtered, and the filtrate was concentrated in vacuum under reduced pressure to give the target product (2.90 g, yield 92%) as a yellow solid. [M+H]⁺ 198.1

### Step 3: tert-butyl(1-(2-chloropyridin-3-yl)cyclopropyl)carbamate

A solution of 1-(2-chloropyridin-3-yl)cyclopropane-1-carboxylic acid (2.90 g, 14.7 mmol), DPPA (6.05 g, 22.0 mmol) and triethylamine (4.40 g, 44 mmol) in tert-butylalcohol (50 mL) was stirred at 85° C. for 48 hours. The reaction solution was poured into water and extracted with ethyl acetate. The organic phase was collected and concentrated in vacuum under reduced pressure, and the resulting residue was purified by column chromatography on silica gel (petroleum ether/ethyl acetate) to give the target product (1.0 g, yield 25%) as a yellow solid. [M+H]⁺ 269.1

### Step 4: tert-butyl(2-((tert-butyldimethylsilyl)oxy)ethyl)(1-(2-chloropyridin-3-yl)cyclopropyl)carbamate

At −5-0° C., 60% sodium hydride (446 mg, 11.2 mmol) was added to a solution of tert-butyl(1-(2-chloropyridin-3-yl)cyclopropyl)carbamate (1.0 g, 3.72 mmol) in DMF (10 mL) and stirred at room temperature for 1 hour. (2-Bromoethyloxy)(tert-butyl)dimethylsilane (1.33 g, 5.58 mmol) was added to the reaction solution and stirred at room temperature for 4 hours. The reaction solution was poured into an aqueous ammonium chloride solution and extracted with ethyl acetate. The organic phase was collected, washed with a saturated aqueous solution of sodium chloride and concentrated in vacuum under reduced pressure, and the resulting residue was purified by column chromatography on silica gel (petroleum ether/ethyl acetate) to give the target product (1.30 g, yield 82%) as a yellow oil. [M+H]⁺ 427.3

### Step 5: tert-butyl(2-((tert-butyldimethylsilyl)oxy)ethyl)(1-(2-((4-methoxybenzyl)amino)pyridin-3-yl)cyclopropyl)carbamate

Tert-butyl(2-((tert-butyldimethylsilyl)oxy)ethyl)(1-(2-chloropyridin-3-yl)cyclopropyl)carbamate (1.30 g, 3.04 mmol), (4-methoxyphenyl)methanamine (835 mg, 6.08 mmol), Pd₂(dba)₃ (278 mg, 0.30 mmol), BINAP (378 mg, 0.61 mmol) and sodium tert-butoxide (630 mg, 9.12 mmol) were added in toluene (10 mL) and stirred at 100° C. for 2 hours under nitrogen protection. The reaction solution was concentrated in vacuum under reduced pressure, and the resulting residue was purified by column chromatography on silica gel (petroleum ether/ethyl acetate) to give the target product (1.50 g, yield 93%) as a yellow solid. [M+H]⁺ 528.4

### Step 6: tert-butyl(2-hydroxyethyl)(1-(2-((4-methoxybenzyl)amino)pyridin-3-yl)cyclopropyl)carbamate

A solution of tert-butyl(2-((tert-butyldimethylsilyl)oxy)ethyl)(1-(2-((4-methoxybenzyl)amino)pyridin-3-yl)cyclopropyl)carbamate (1.50 g, 2.84 mmol) in trifluoroacetic acid (5 mL) was stirred at 50° C. for 3 hours and concentrated in vacuum under reduced pressure. The resulting residue was dissolved with dichloromethane and washed with an aqueous sodium bicarbonate solution, and the organic phase was collected, dried with anhydrous sodium sulfate, filtered and concentrated in vacuum under reduced pressure to give the target product (0.75 g, yield 84%) as a yellow solid. [M+H]⁺ 314.2

### Step 7: 3-(1-(5-chloro-4-fluoro-2-(methylthio)-8,9-dihydro-1OH-7-oxa-1,3,6,10-tetraazacyclohepta[de]naphthalen-10-yl)cyclopropyl)-N-(4-methoxybenzyl)pyridin-2-amine

The target product was prepared from corresponding intermediates and reagents by following the preparation steps of the intermediate I-C3. [M+H]⁺ 539.2

### Intermediates I-C29 and I-C30 3-(1-((S)-5-chloro-4-fluoro-9-methyl-2-(methylthio)-8,9-dihydro-1OH-7-oxa-1,3,6,10-tetraazacyclohepta[de]naphthalen-10-yl)ethyl)pyridin-2-amine diastereomers

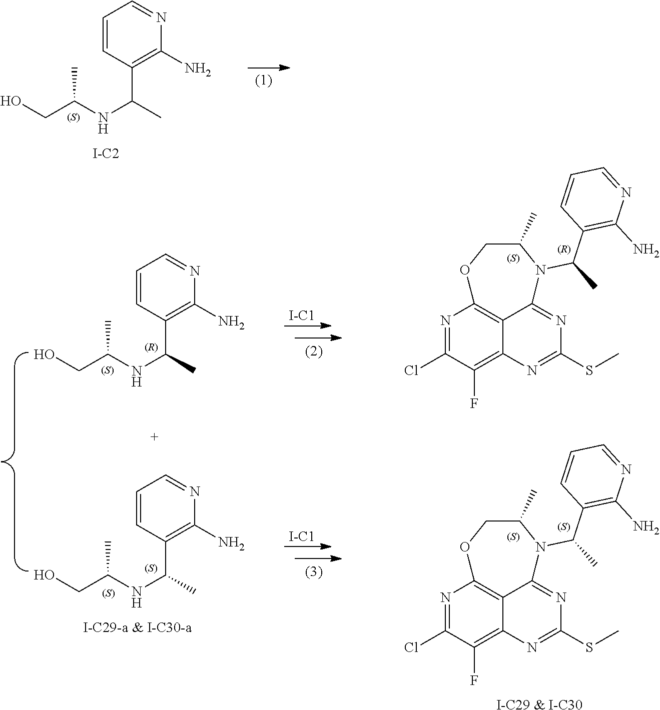

Intermediates I-C29-a and I-C30-a were prepared respectively from corresponding chiral raw materials and reagents by following the preparation step 1 of the intermediates I-C8 and I-C9. The chiral HPLC analysis was performed under the following conditions (flow rate: 1 mL/minute; detector: UV 254 nm):

| Intermediate | Column | Mobile phase | RT/minute | de % |
|---|---|---|---|---|
| I-C29-a | ODH | n-Heptane/ | 6.149 | 100% |
| I-C30-a | (0.46 × 25 cm) | ethanol (0.1% | 7.215 | 100% |
| | | diethylamine) = | | |
| | | 90:10 | | |

With the internal temperature being maintained lower than 40° C., sodium tert-butoxide (126.26 g, 1.31 mol) was slowly added in batches to a solution of the intermediate I-C29-a (80.17 g, 0.41 mol) and the intermediate I-C1 (92.0 g, 0.33 mol) in tetrahydrofuran (730 mL) and stirred at room temperature for 2 hours. The reaction solution was poured into water (2.1 L), adjusted with 1.2 M hydrochloric acid to pH=7, and extracted with dichloromethane (2.1 L×3). The organic phase was collected, combined and concentrated in vacuum under reduced pressure, and BOP-Cl (177.1 g, 0.42 mol) and DMAP (128.5 g, 1.05 mol) were added to a solution of the resulting residue in DMA and stirred at room temperature for 16 hours. The reaction solution was stirred at 100° C. for 6 hours, poured into water and filtered, and the cake was collected. The cake was dried in vacuum under reduced pressure, slurried in methanol (400 mL) and filtered, and the solid was collected and dried under reduced pressure to give the intermediate I-C29 (83.2 g, yield 57%). [M+H]⁺ 421.1

The intermediate I-C30 was prepared from the intermediate I-C30-a and corresponding reagents by following the preparation steps of the intermediate I-C29.

The intermediates I-C38 and I-C39 were prepared from the corresponding starting materials and reagents by following the preparation steps of the intermediates I-C29 and I-C30:

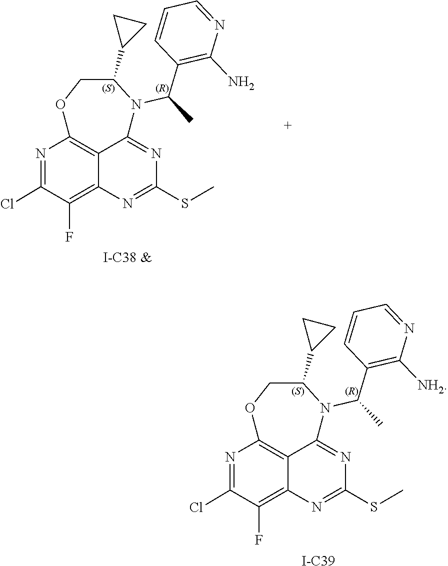

### Intermediate I-C32 (*)tert-butyl ((2S)-1-((3-(1-((S)-5-chloro-4-fluoro-9-methyl-2-(methylthio)-8,9-dihydro-1OH-7-oxa-1,3,6,10-tetraazacyclohepta[de]naphthalen-10-yl)ethyl)pyridin-2-yl)amino)-1-oxopropan-2-yl)carbamate

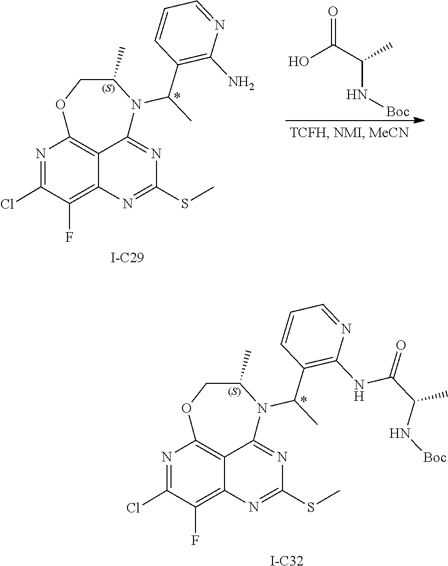

NMI (0.98 g, 11.88 mmol) and TCFH (2.0 g, 7.13 mmol) were added to a solution of the intermediate I-C29 (1.00 g, 2.38 mmol) and (tert-butoxycarbonyl)-L-alanine (0.90 g, 4.75 mmol) in acetonitrile (15 mL) and stirred at room temperature for 16 hours under nitrogen protection. The reaction solution was poured into water and extracted with ethyl acetate, the organic phase was collected and concentrated in vacuum under reduced pressure, and the resulting residue was purified by column chromatography on silica gel (water/methanol) to give the target intermediate (1.36 g, yield 97%) as a solid. [M+H]⁺ 592.1.

### Compound 1 4-(10-(1-(2-Aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-5,6-difluoronaphthalen-2-ol

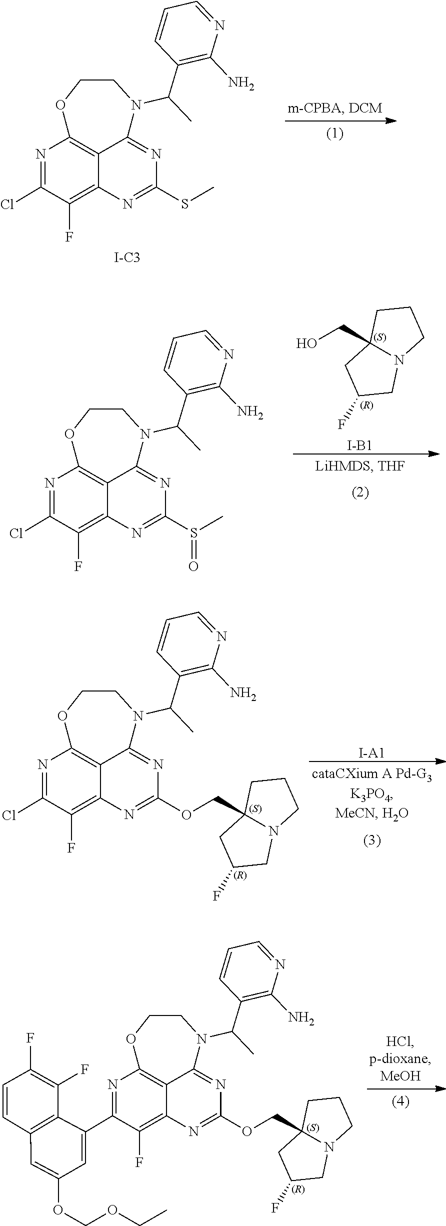

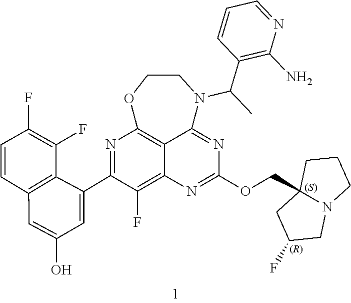

### Step 1: 3-(1-(5-chloro-4-fluoro-2-(methylsulfinyl)-8,9-dihydro-1OH-7-oxa-1,3,6,10-tetraazacyclohepta[de]naphthalen-10-yl)ethyl)pyridin-2-amine

At 0-5° C., m-CPBA (at a content of 85%, 384 mg, 1.89 mmol) was added to a solution of the intermediate I-C3 (700 mg, 1.72 mmol) in dichloromethane (10 mL) and stirred for 30 minutes. Water was added to the reaction solution and extracted with dichloromethane (10 mL×3). The organic phase was collected and concentrated in vacuum under reduced pressure to give a crude target product (700 mg), which was used in the reaction of the next step directly.

### Step 2: 3-(1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

At 0-5° C., a 1.3 M LiHMDS/THF solution (3.31 mL, 4.3 mmoL) was added dropwise to a solution of 3-(1-(5-chloro-4-fluoro-2-(methylsulfinyl)-8,9-dihydro-1OH-7-oxa-1,3,6,10-tetraazacyclohepta[de]naphthalen-10-yl)ethyl)pyridin-2-amine (700 mg) and ((2R,7aS)-2-fluorotetrahydro-1H-pyrrolizin-7a(5H)-yl)methanol (intermediate I-B1, 821 mg, 5.16 mmoL) in anhydrous tetrahydrofuran (8 mL) and stirred for 30 minutes. Water was added to the reaction solution and extracted with dichloromethane (10 mL×3). The organic phase was collected and concentrated in vacuum under reduced pressure, and the resulting residue was purified by column chromatography on silica gel (dichloromethane/methanol) to give the target product (210 mg, two-step yield 24%) as a pale yellow solid. [M+H]⁺ 518.2.

### Step 3: 3-(1-(5-(3-(ethoxymethoxy)-7,8-difluoronaphthalen-1-yl)-4-fluoro-2-(((2R,7aS)-2-fluorotetrahydro-1H-pyrrolizin-7a(5H)-yl)methoxy)-8,9-dihydro-1OH-7-oxa-1,3,6,10-tetraazacyclohepta[de]naphthalen-10-yl)ethyl)pyridin-2-amine

3-(1-(5-Chloro-4-fluoro-2-(((2R,7aS)-2-fluorotetrahydro-1H-pyrrolizin-7a(5H)-yl)methoxy)-8,9-dihydro-1OH-7-oxa-1,3,6,10-tetraazacyclohepta[de]naphthalen-10-yl)ethyl)pyridin-2-amine (47 mg, 0.09 mmol), the intermediate I-A1 (66 mg, 0.18 mmol), cataCXium A Pd—G₃ (7 mg, 0.01 mmol) and anhydrous potassium phosphate (57 mg, 0.27 mmol) were dissolved in acetonitrile/water (5 mL/1 mL) and stirred at 70° C. for 3 hours under nitrogen protection. The reaction solution was concentrated in vacuum under reduced pressure, and the resulting residue was purified by column chromatography on silica gel (water/methanol) to give the target product as a yellow solid.

### Step 4: 4-(10-(1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-5,6-difluoronaphthalen-2-ol

At room temperature, a 4 M hydrogen chloride/1,4-dioxane solution (4 mL) was added to a solution of 3-(1-(5-(3-(ethoxymethoxy)-7,8-difluoronaphthalen-1-yl)-4-fluoro-2-(((2R,7aS)-2-fluorotetrahydro-1H-pyrrolizin-7a(5H)-yl)methoxy)-8,9-dihydro-1OH-7-oxa-1,3,6,10-tetraazacyclohepta[de]naphthalen-10-yl)ethyl)pyridin-2-amine in methanol (0.5 mL) and stirred for 30 minutes. After concentration in vacuum under reduced pressure, water (1 mL) and concentrated ammonia water (0.5 mL) were added to the resulting residue and extracted with dichloromethane, and the organic phase was collected and concentrated in vacuum under reduced pressure. The resulting residue was purified by column chromatography on silica gel (dichloromethane/methanol) to give the target product (37 mg, two-step yield 62.7%) as a pale yellow solid. [M+H]⁺ 662.6. ¹H NMR (400 MHz, CD₃OD) δ 7.95 (d, J=3.9 Hz, 1H), 7.76 (d, J=7.3 Hz, 1H), 7.58 (dd, J=9.2, 5.1 Hz, 1H), 7.38 (dd, J=17.4, 9.2 Hz, 1H), 7.29 (d, J=2.1 Hz, 1H), 7.19 (dd, J=4.5, 2.3 Hz, 1H), 6.78 (dd, J=7.5, 5.1 Hz, 1H), 6.62 (d, J=6.8 Hz, 1H), 5.43-5.21 (m, 1H), 4.49-4.29 (m, 4H), 3.84-3.74 (m, 1H), 3.62-3.51 (m, 1H), 3.28-3.17 (m, 3H), 3.02 (s, 1H), 2.40-2.13 (m, 3H), 2.02-1.89 (m, 3H), 1.68-1.64 (m, 3H).

The compounds in the table below were prepared from corresponding intermediates and reagents by following the preparation steps of the compound 1:

| Com- | | LC-MS | |
|---|---|---|---|
| pound | Structural formula | [M + H]⁺ | ¹HNMR |
| 4 | 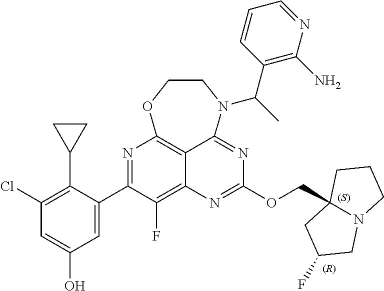 | 651.3 | (400 MHz, CD₃OD) δ 7.96-7.86 (m, 1H), 7.76-7.67 (m, 1H), 6.80-6.70 (m, 2H), 6.60-6.51 (m, 2H), 5.41- 5.15 (m, 1H), 4.51-4.15 (m, 4H), 3.77-3.63 (m, 1H), 3.58-3.43 (m, 1H), 3.19-3.11 (m, 2H), 3.02-2.91 (m, 1H), 2.30-2.08 (m, 3H), 1.98-1.83 (m, 3H), 1.79-1.69 (m, 1H), 1.63-1.49 (m, 4H), 0.63-0.48 (m, 2H), 0.16- 0.00 (m 2H). |
| 15 | 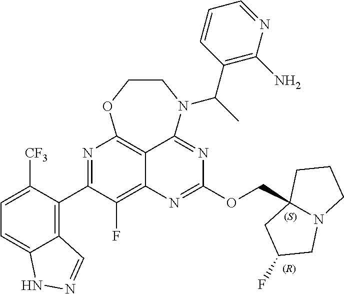 | 668.3 | (400 MHz, CD₃OD) δ 7.96 (d, J = 4.3 Hz, 1H), 7.86- 7.74 (m, 4H), 6.78 (dd, J = 7.4, 5.2 Hz, 1H), 6.67- 6.57 (m, 1H), 5.41-5.21 (m, 1H), 4.51-4.27 (m, 4H), 3.86-3.74 (m, 1H), 3.64-3.52 (m, 1H), 3.28-3.16 (m, 3H), 3.07-2.96 (m, 1H), 2.42-2.12 (m, 3H), 2.04-1.88 (m, 3H), 1.69-1.64 (m, 3H). |
| 18 | 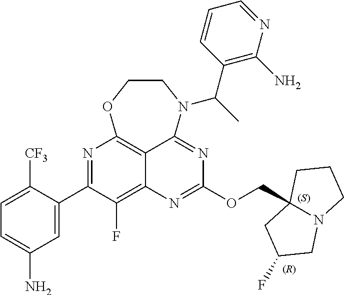 | 643.2 | (400 MHz, CD₃OD) δ 7.97 (s, 1H), 7.76 (d, J = 7.1 Hz, 1H), 7.44 (d, J = 8.7 Hz, 1H), 6.78 (d, J = 7.7 Hz, 2H), 6.61 (s, 1H), 6.55 (d, J = 7.2 Hz, 1H), 5.53-5.43 (m, 1H), 4.58-4.44 (m, 4H), 4.35-4.31 (m, 1H), 3.75-3.71 (m, 2H), 3.62-3.56 (m, 4H), 2.48-2.41 (m, 2H), 2.20- 2.15 (m, 2H), 2.02-1.98 (m, 2H), 1.64 (d, J = 6.8 Hz, 3H). |
| 25 | 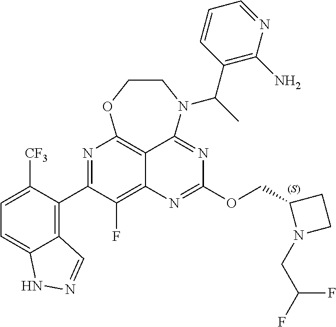 | 660.3 | (400 MHz, CD₃OD) δ 7.98-7.93 (m, 1H), 7.83-7.75 (m, 4H), 6.82-6.73 (m, 1H), 6.68-6.56 (m, 1H), 6.01- 5.68 (m, 1H), 4.62-4.31 (m, 4H), 3.82-3.73 (m, 2H), 3.62-3.49 (m, 2H), 3.25-3.11 (m, 2H), 2.88-2.75 (m, 1H), 2.21-2.13 (m, 2H), 1.69-1.64 (m, 3H). |
| 36 | 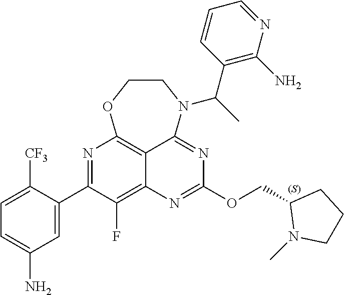 | 599.3 | (400 MHz, CD₃OD) δ 7.94 (d, J = 4.1 Hz, 1H), 7.74 (d, J = 6.9 Hz, 1H), 7.44 (d, J = 8.7 Hz, 1H), 6.77 (dd, J = 7.6, 5.2 Hz, 2H), 6.65-6.55 (m, 2H), 4.61-4.49 (m, 2H), 4.47-4.25 (m, 2H), 3.78-3.68 (m, 1H), 3.57- 3.45 (m, 1H), 3.24-3.14 (m, 1H), 3.06-2.94 (m, 1H), 2.65-2.58 (m, 3H), 2.516-2.45 (m, 1H), 2.21-2.08 (m, 1H), 1.92-1.79 (m, 3H), 1.66-1.60 (d, J = 6.9 Hz, 3H). |
| 37 | 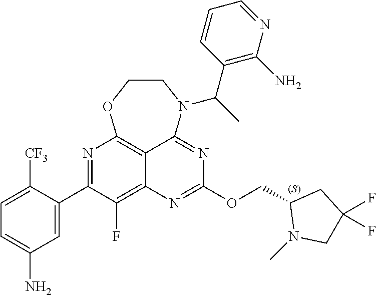 | 635.2 | (400 MHz, CD₃OD) δ 7.94 (d, J = 5.1 Hz, 1H), 7.74 (d, J = 7.6 Hz, 1H), 7.44 (d, J = 8.6 Hz, 1H), 6.77 (dd, J = 7.5, 5.5 Hz, 2H), 6.64-6.56 (m, 2H), 4.66-4.51 (m, 2H), 4.47-4.26 (m, 2H), 3.76-3.66 (m, 1H), 3.55- 3.46 (m, 1H), 3.43-3.34 (m, 1H), 3.15-3.02 (m, 1H), 2.80-2.65 (m, 1H), 2.61-2.51 (m, 1H), 2.50-2.46 (m, 3H), 2.40-2.22 (m, 1H), 1.68-1.58 (m, 3H). |
| 40 | 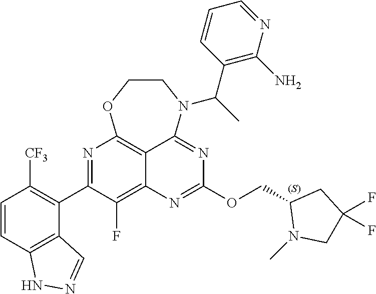 | 660.3 | (400 MHz, CD₃OD) δ 8.03-7.89 (m, 1H), 7.86-7.70 (m, 4H), 6.86-6.72 (m, 1H), 6.71-6.53 (m, 1H), 4.67- 4.55 (m, 2H), 4.51-4.42 (m, 1H), 4.41-4.30 (m, 1H), 3.88-3.66 (m, 1H), 3.63-3.51 (m, 1H), 3.44-3.32 (m, 2H), 3.12-3.03 (m, 1H), 2.84-2.64 (m, 1H), 2.48 (s, 3H), 2.39-2.23 (m, 1H), 1.67 (s, 3H). |
| 59 | 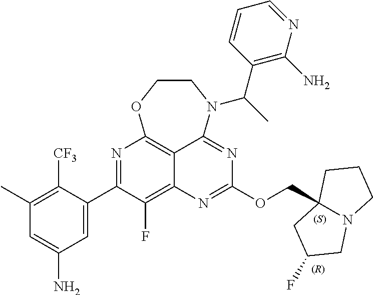 | 657.2 | (400 MHz, CD₃OD) δ 7.98-7.92 (m, 1H), 7.78-7.69 (m, 1H), 6.80-6.73 (m, 1H), 6.69-6.65 (m, 1H), 6.62- 6.53 (m, 1H), 6.42-6.36 (m 1H), 5.49-5.16 (m, 1H), 4.46-4.19 (m, 4H), 3.82-3.67 (m, 1H), 3.58-3.45 (m, 1H), 3.27-3.18 (m, 3H), 3.08-2.97 (m, 1H), 2.43-2.37 (m, 3H), 2.36-2.10 (m, 3H), 2.04-1.86 (m, 3H), 1.70- 1.58 (m, 3H). |
| 60 | 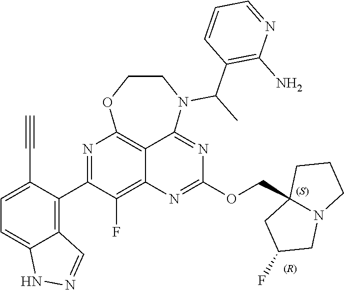 | 624.2 | (400 MHz, CD₃OD) δ 7.98-7.93 (m, 1H), 7.90-7.84 (m, 1H), 7.78-7.73 (m, 1H), 7.67-7.56 (m, 2H), 6.82- 6.74 (m, 1H), 6.65-6.59 (m, 1H), 5.41-5.23 (m, 1H), 4.62-4.26 (m, 4H), 3.84-3.72 (m, 1H), 3.61-3.52 (m, 1H), 3.39 (s, 1H), 3.27-3.19 (m, 2H), 3.10-2.95 (m, 1H), 2.45-1.83 (m, 7H), 1.70-1.61 (m, 3H). |
| 63 | 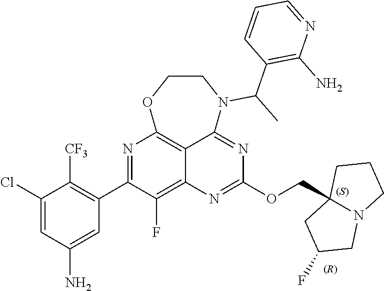 | 677.2 | (400 MHz, CD₃OD) δ 7.94 (d, J = 4.7 Hz, 1H), 7.74 (d, J = 7.7 Hz, 1H), 6.86 (d, J = 2.1 Hz, 1H), 6.77 (dd, J = 7.5, 5.2 Hz, 1H), 6.58 (d, J = 7.0 Hz, 1H), 6.46 (s, 1H), 5.39-5.21 (m, 1H), 4.50-4.23 (m, 4H), 3.79-3.47 (m, 2H), 3.28-3.17 (m, 3H), 3.08-2.94 (m, 1H), 2.40- 1.90 (m, 6H), 1.63 (d, J = 6.9 Hz, 3H). |
| 64 | 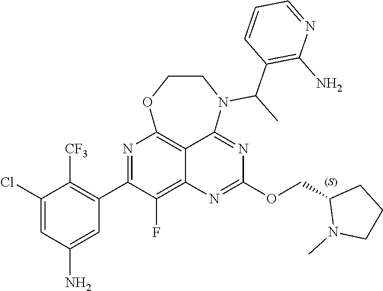 | 633.2 | (400 MHz, CD₃OD) δ 7.94 (d, J = 5.0 Hz, 1H), 7.74 (d, J = 7.8 Hz, 1H), 6.86 (d, J = 2.0 Hz, 1H), 6.81- 6.73 (m, 1H), 6.60 (d, J = 5.9 Hz, 1H), 6.46 (s, 1H), 4.59-4.24 (m, 4H), 3.74-3.47 (m, 2H), 3.14-3.06 (m, 1H), 2.88-2.78 (m, 1H), 2.52 (s, 3H), 2.43-2.32 (m, 1H), 2.20-2.07 (m, 1H), 1.90-1.74 (m, 3H), 1.64 (d, J = 6.9 Hz, 3H). |
| 66 | 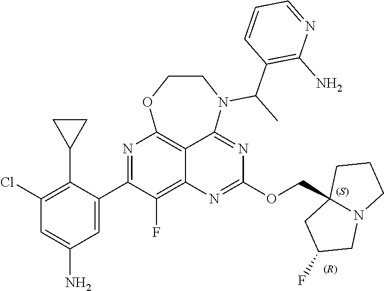 | 649.3 | (400 MHz, CD₃OD) δ 7.97-7.93 (m, 1H), 7.76-7.73 (m, 1H), 6.84-6.80 (m, 1H), 6.79-6.75 (m, 1H), 6.64- 6.55 (m, 2H), 5.41-5.21 (m, 1H), 4.47-4.41 (m, 1H), 4.37-4.25 (m, 3H), 3.78-3.69 (m, 1H), 3.56-3.49 (m, 1H), 3.25-3.18 (m, 2H), 3.06-2.96 (m, 1H), 2.41-2.09 (m, 3H), 2.03-1.74 (m, 4H), 1.68-1.56 (m, 4H), 0.71- 0.49 (m, 2H), 0.15-0.03 (s, 2H). |
| 162 | 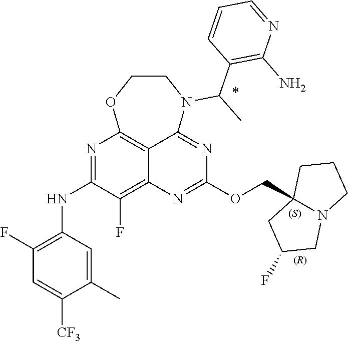 | 675.3 | (400 MHz, CD₃OD) δ 8.01 (d, J = 7.8 Hz, 1H), 7.89 (d, J = 4.8 Hz, 1H), 7.67 (d, J = 7.4 Hz, 1H), 7.34 (d, J = 11.4 Hz, 1H), 6.76-6.68 (m, 1H), 6.54 (q, J = 6.7 Hz, 1H), 5.38-5.16 (m, 1H), 4.38-4.14 (m, 4H), 3.67- 3.59 (m, 1H), 3.44-3.36 (m, 1H), 3.26-3.08 (m, 3H), 3.02-2.94 (m, 1H), 2.44-2.04 (m, 6H), 2.00-1.80 (m, 3H), 1.56 (d, J = 6.9 Hz, 3H). |
| 259 | 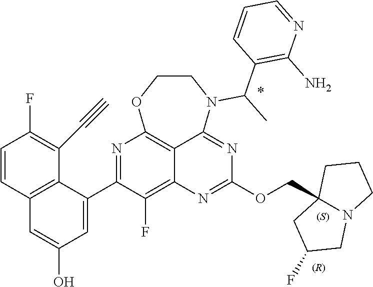 | 668.3 | (400 MHz, CD₃OD) δ 7.94 (d, J = 5.0 Hz, 1H), 7.86- 7.77 (m, 1H), 7.79-7.70 (m, 1H), 7.36-7.24 (m, 2H), 7.18 (dd, J = 4.7, 2.6 Hz, 1H), 6.76 (ddd, J = 7.5, 5.2, 2.0 Hz, 1H), 6.59 (t, J = 6.9 Hz, 1H), 5.29 (d, J = 53.8 Hz, 1H), 4.52-4.26 (m, 4H), 3.83-3.69 (m, 1H), 3.63- 3.54 (m, 1H), 3.52-3.41 (m, 1H), 3.29-3.16 (m, 3H), 3.07-2.95 (m, 1H), 2.40-1.88 (m, 6H), 1.65 (dd, J = 6.9, 3.3 Hz, 3H). |
| 314 | 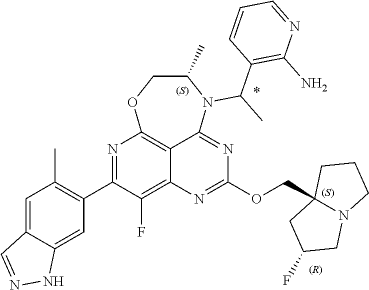 | 628.3 | (400 MHz, CD₃OD) δ 8.06-7.91 (m, 2H), 7.80 (d, J = 7.2 Hz, 1H), 7.74-7.65 (m, 1H), 7.54 (s, 1H), 6.85- 6.67 (m, 2H), 5.43-5.22 (m, 1H), 4.66-4.59 (m, 1H), 4.45-4.24 (m, 3H), 4.13-4.01 (m, 1H), 3.43-3.30 (m, 3H), 3.15-3.02 (m, 1H), 2.46-2.14 (m, 6H), 2.11-1.85 (m, 3H), 1.68 (d, J = 6.8 Hz, 3H), 0.70 (d, J = 6.8 Hz, 3H). |
| 315 | 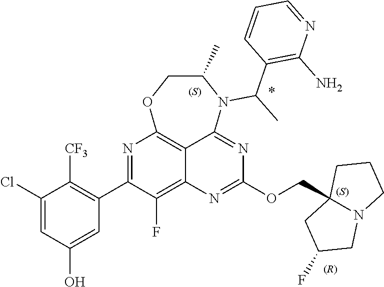 | 692.2 | (400 MHz, CD₃OD) δ 7.99-7.93 (m, 1H), 7.83-7.76 (m, 1H), 7.10-7.05 (m, 1H), 6.86-6.66 (m, 3H), 5.31 (m, 1H), 4.65-4.56 (m, 1H), 4.43-4.24 (m, 3H), 4.11- 4.00 (m, 1H), 3.37-3.31 (m, 2H), 3.24-3.20 (m, 1H), 3.09-2.99 (m, 1H), 2.34-2.13 (m, 3H), 2.07-1.88 (m, 3H), 1.66 (d, J = 6.8 Hz, 3H), 0.68 (d, J = 6.8 Hz, 3H). |
| 316 | 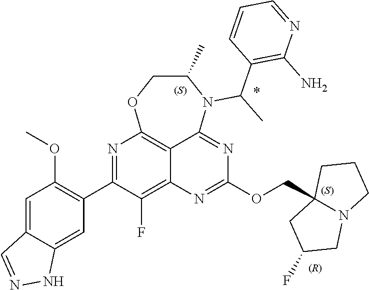 | 644.3 | (400 MHz, CD₃OD) δ 8.04-7.99 (m, 1H), 7.99-7.93 (m, 1H), 7.82-7.77 (m, 1H), 7.61-7.52 (m, 1H), 7.39- 7.29 (m, 1H), 6.81-6.72 (m, 2H), 5.38-5.22 (m, 1H), 4.64-4.59 (m, 1H), 4.38-4.26 (m, 3H), 4.11-4.01 (m, 1H), 3.88-3.78 (m, 3H), 3.27-3.10 (m, 3H), 3.06-2.96 (m, 1H), 2.42-2.10 (m, 3H), 2.04-1.87 (m, 3H), 1.74- 1.59 (m, 3H), 0.76-0.65 (m, 3H). |
| 317 | 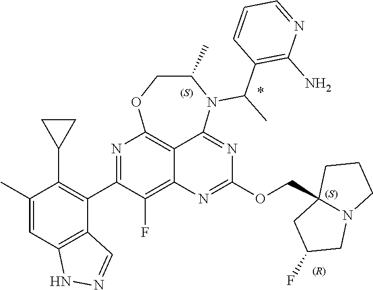 | 668.4 | (400 MHz, CD₃OD) δ 8.00-7.94 (m, 1H), 7.83-7.79 (m, 1H), 7.79-7.62 (m, 1H), 7.48-7.43 (m, 1H), 6.84- 6.72 (m, 2H), 5.45-5.16 (m, 1H), 4.70-4.58 (m, 1H), 4.41-4.28 (m, 3H), 4.12-4.04 (m, 1H), 3.28-3.20 (m, 2H), 3.19-3.14 (m, 1H), 3.05-2.96 (m, 1H), 2.70-2.61 (m, 3H), 2.37-2.12 (m, 3H), 2.05-1.91 (m, 4H), 1.68 (d, 3H), 0.92-0.80 (m, 1H), 0.76-0.69 (m, 3H), 0.67- 0.56 (m, 1H), 0.25-0.16 (m, 1H), 0.12-0.03 (m, 1H). |
| 318 | 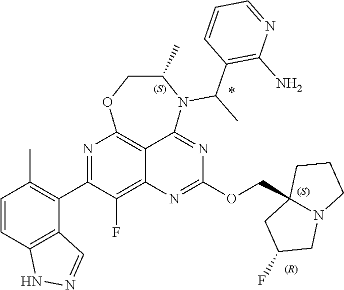 | 628.4 | (400 MHz, CD₃OD) δ 8.03-7.91 (m, 1H), 7.85-7.77 (m, 1H), 7.76-7.63 (m, 1H), 7.60-7.50 (m, 1H), 7.41- 7.31 (m, 1H), 6.85-6.69 (m, 2H), 5.42-5.14 (m, 1H), 4.68-4.49 (m, 2H), 4.42-4.25 (m, 3H), 4.13-3.98 (m, 1H), 3.25-3.15 (m, 2H), 3.07-2.95 (m, 1H), 2.44-2.09 (m, 6H), 2.05-1.82 (m, 3H), 1.74-1.59 (m, 3H), 0.82- 0.64 (m, 3H). |
| 319 | 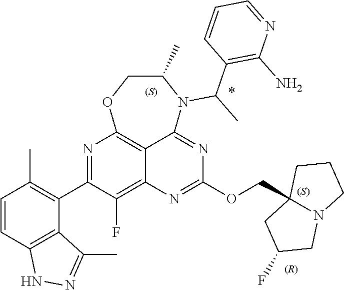 | 642.4 | (400 MHz, CD₃OD) δ 8.02-7.91 (m, 1H), 7.83-7.73 (m, 1H), 7.50-7.42 (m, 1H), 7.37-7.28 (m, 1H), 6.83- 6.71 (m, 2H), 5.42-5.15 (m, 1H), 4.65-4.59 (m, 1H), 4.38-4.28 (m, 3H), 4.11-4.03 (m, 1H), 3.31-3.27 (m, 2H), 3.27-3.17 (m, 2H), 3.17-3.12 (m, 1H), 3.06-2.92 (m, 1H), 2.33-2.15 (m, 5H), 2.03-1.82 (m, 5H), 1.72- 1.62 (m, 3H), 0.73-0.66 (m, 3H). |
| 320 | 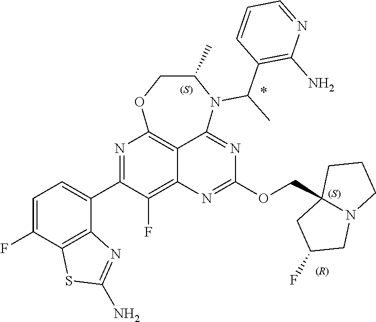 | 664.3 | (400 MHz, CD₃OD) δ 8.00-7.89 (m, 1H), 7.85-7.70 (m, 1H), 7.46-7.33 (m, 1H), 7.06-6.87 (m, 1H), 6.84- 6.65 (m, 2H), 5.44-5.17 (m, 1H), 4.64-4.55 (m, 1H), 4.42-4.14 (m, 3H), 4.12-3.97 (m, 1H), 3.38-3.30 (m, 2H), 3.25-3.18 (m, 1H), 3.09-2.96 (m, 1H), 2.43-2.10 (m, 3H), 2.08-1.79 (m, 3H), 1.67 (d, J = 6.8 Hz, 3H), 0.70 (d, J = 6.8 Hz, 3H). |
| 321 | 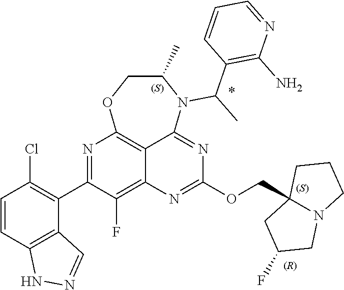 | 648.3 | (400 MHz, CD₃OD) δ 7.99-7.92 (m, 1H), 7.85-7.45 (m, 4H), 6.83-6.72 (m, 2H), 5.42-5.18 (m, 1H), 4.69- 4.56 (m, 1H), 4.38-4.24 (m, 3H), 4.15-4.01 (m, 1H), 3.27-3.14 (m, 3H), 3.05-2.92 (m, 1H), 2.40-2.13 (m, 3H), 2.05-1.87 (m, 3H), 1.73-1.64 (m, 3H), 0.80-0.67 (m, 3H). |
| 322 | 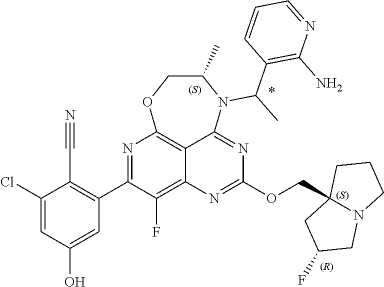 | 649.3 | (400 MHz, CD₃OD) δ 8.02-7.50 (m, 2H), 6.82-6.70 (m, 2H), 6.67-6.64 (m, 1H), 6.62-6.58 (m, 1H), 5.42- 5.18 (m, 1H), 4.66-4.58 (m, 1H), 4.40-4.22 (m, 3H), 4.04 (dd, J = 7.1, 5.3 Hz, 1H), 3.27-3.14 (m, 3H), 3.05-2.93 (m, 1H), 2.42-2.11 (m, 3H), 2.05-1.89 (m, 3H), 1.75-1.63 (m, 3H), 0.68 (d, J = 6.9 Hz, 3H). |
| 323 | 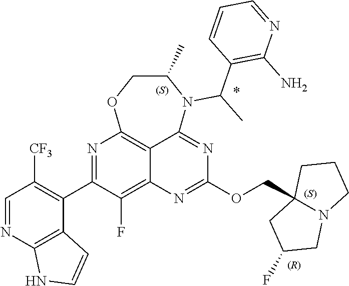 | 682.3 | (400 MHz, CD₃OD) δ 8.71-8.56 (m, 1H), 8.03-7.91 (m, 1H), 7.86-7.77 (m, 1H), 7.64-7.52 (m, 1H), 6.85- 6.71 (m, 2H), 6.39-6.17 (m, 1H), 5.43-5.19 (m, 1H), 4.70-4.57 (m, 1H), 4.41-4.28 (m, 3H), 4.16-4.02 (m, 1H), 3.26-3.13 (m, 3H), 3.09-2.97 (m, 1H), 2.39-2.12 (m, 3H), 2.09-1.89 (m, 3H), 1.71-1.65 (m, 3H), 0.79- 0.67 (m, 3H). |
| 324 | 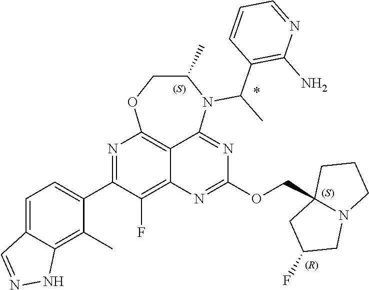 | 628.4 | (400 MHz, CD₃OD) δ 8.08 (s, 1H), 8.00-7.92 (m, 1H), 7.83-7.75 (m, 1H), 7.73-7.65 (m, 1H), 7.22-7.11 (m, 1H), 6.83-6.69 (m, 2H), 5.41-5.17 (m, 1H), 4.65- 4.59 (m, 1H), 4.37-4.27 (m, 3H), 4.11-4.02 (m, 1H), 3.28-3.16 (m, 3H), 3.06-2.94 (m, 1H), 2.53-2.41 (m, 3H), 2.38-2.13 (m, 3H), 2.03-1.88 (m, 3H), 1.72-1.62 (m, 3H), 0.74-0.65 (m, 3H). |
| 325 | 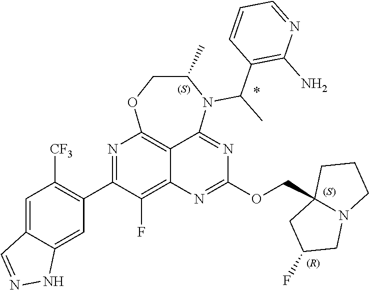 | 682.3 | (400 MHz, CD₃OD) δ 8.35 (d, J = 0.8 Hz, 1H), 8.29 (s, 1H), 8.01-7.92 (m, 1H), 7.85-7.74 (m, 1H), 7.67 (s, 1H), 6.77 (m, 2H), 5.41-5.20 (m, 1H), 4.65-4.58 (m, 1H), 4.42-4.25 (m, 3H), 4.11-4.00 (m, 1H), 3.36- 3.30 (m, 1H), 3.27-3.14 (m, 2H), 3.09-2.96 (m, 1H), 2.45-2.08 (m, 3H), 2.08-1.81 (m, 3H), 1.68 (d, J = 6.8 Hz, 3H), 0.70 (d, J = 6.9 Hz, 3H). |
| 327 | 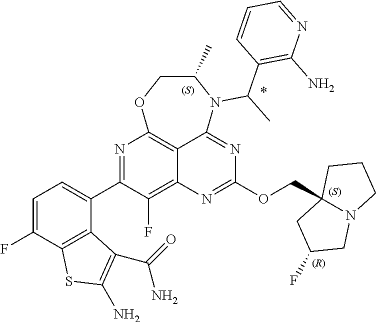 | 706.3 | (400 MHz, CD₃OD) δ 8.01-7.93 (m, 1H), 7.84-7.75 (m, 1H), 7.50-7.41 (m, 1H), 7.00-6.90 (m, 1H), 6.81- 6.71 (m, 2H), 5.41-5.19 (m, 1H), 4.62-4.54 (m, 1H), 4.33-4.23 (m, 3H), 4.08-4.00 (m, 1H), 3.26-3.14 (m, 3H), 3.03-2.96 (m, 1H), 2.37-2.14 (m, 3H), 2.02-1.89 (m, 3H), 1.65 (d, J = 6.9 Hz, 3H), 0.71 (d, J = 6.9 Hz, 3H). |
| 328 | 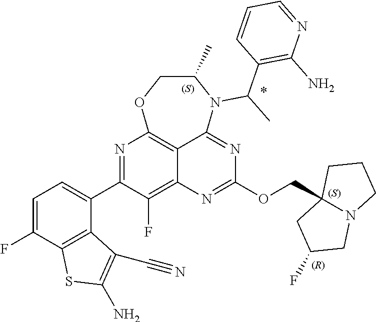 | 688.3 | (400 MHz, CD₃OD) δ 7.99-7.92 (m, 1H), 7.81-7.75 (m, 1H), 7.42-7.35 (m, 1H), 7.05-6.96 (m, 1H), 6.80- 6.70 (m, 2H), 5.41-5.15 (m, 1H), 4.64-4.56 (m, 1H), 4.37-4.24 (m, 3H), 4.09-4.01 (m, 1H), 3.27-3.17 (m, 2H), 3.17-3.12 (m, 1H), 3.04-2.94 (m, 1H), 2.38-2.12 (m, 3H), 2.03-1.87 (m, 3H), 1.65 (d, J = 6.8 Hz, 3H), 0.71 (d, J = 6.9 Hz, 3H). |
| 332 | 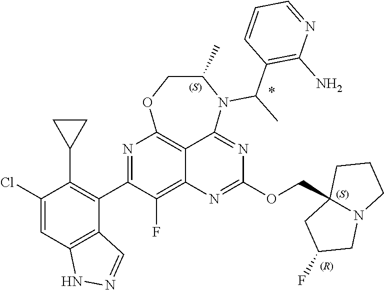 | 688.3 | (400 MHz, CD₃OD) δ 8.00-7.95 (m, 1H), 7.89-7.64 (m, 3H), 6.82-6.72 (m, 2H), 5.43-5.23 (m, 1H), 4.70- 4.60 (m, 1H), 4.45-4.34 (m, 3H), 4.16-4.04 (m, 1H), 3.48-3.31 (m, 3H), 3.12-3.03 (m, 1H), 2.48-2.14 (m, 3H), 2.12-1.94 (m, 4H), 1.69 (d, J = 6.8 Hz, 3H), 0.98-0.05 (m, 7H). |
| 333 | 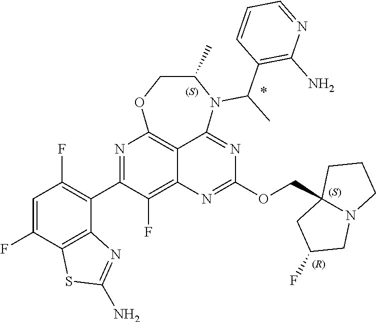 | 681.8 | (400 MHz, CD₃OD) δ 8.02-7.91 (m, 1H), 7.84-7.73 (m, 1H), 6.93-6.68 (m, 3H), 5.42-5.17 (m, 1H), 4.67- 4.56 (m, 1H), 4.44-4.21 (m, 3H), 4.09-4.01 (m, 1H), 3.36-3.30 (m, 1H), 3.27-3.13 (m, 2H), 3.08-2.93 (m, 1H), 2.43-2.07 (m, 3H), 2.06-1.81 (m, 3H), 1.66 (d, J = 6.9 Hz, 3H), 0.79-0.57 (m, 3H). |
| 337 | 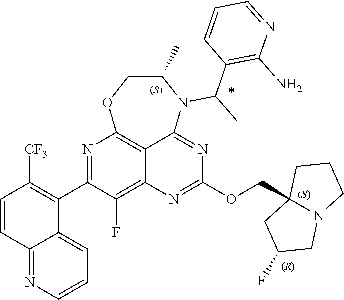 | 693.4 | (400 MHz, CD₃OD) δ 9.10-8.95 (m, 1H), 8.37 (d, J = 9.0 Hz, 1H), 8.24-8.13 (m, 1H), 8.13-7.76 (m, 3H), 7.68-7.50 (m, 1H), 6.86-6.72 (m, 2H), 5.41-5.17 (m, 1H), 4.69-4.59 (m, 1H), 4.46-4.19 (m, 3H), 4.18-4.02 (m, 1H), 3.28-3.12 (m, 3H), 3.03-2.95 (m, 1H), 2.42- 2.09 (m, 3H), 2.04-1.83 (m, 3H), 1.78-1.61 (m, 3H), 0.81-0.68 (m, 3H). |
| 360 | 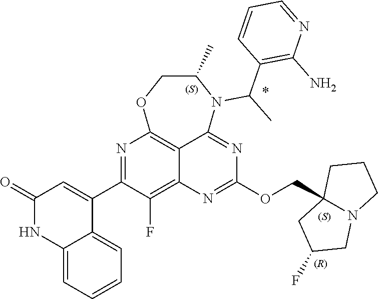 | 641.2 | (400 MHz, CD₃OD) δ 8.02-7.91 (m, 1H), 7.85-7.73 (m, 1H), 7.63-7.50 (m, 1H), 7.50-7.38 (m, 2H), 7.27- 7.14 (m, 1H), 6.82-6.68 (m, 3H), 5.41-5.19 (m, 1H), 4.68-4.58 (m, 1H), 4.42-4.27 (m, 3H), 4.15-4.01 (m, 1H), 3.29-3.15 (m, 3H), 3.08-2.96 (m, 1H), 2.42-2.07 (m, 3H), 2.05-1.81 (m, 3H), 1.68 (d, J = 6.9 Hz, 3H), 0.72 (d, J = 6.8 Hz, 3H). |
| 342 | 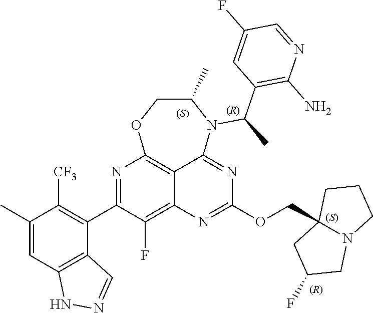 | 714.4 | (400 MHz, CD₃OD) δ 7.93-7.89 (m, 1H), 7.75-7.58 (m, 3H), 6.82-6.73 (m, 1H), 5.38-5.21 (m, 1H), 4.67- 4.60 (m, 1H), 4.40-4.28 (m, 3H), 4.16-4.07 (m, 1H), 3.29-3.16 (m, 3H), 3.06-2.97 (m, 1H), 2.72-2.64 (m, 3H), 2.40-2.11 (m, 3H), 2.04-1.86 (m, 3H), 1.75-1.65 (m, 3H), 0.82-0.71 (m, 3H). |
| | + | | |
| 364 | 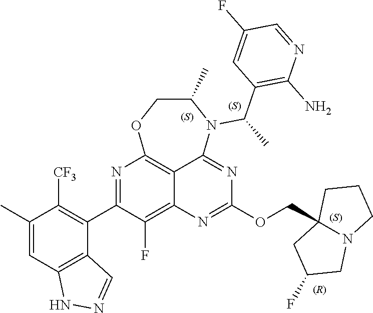 | 714.2 | (400 MHz, CD₃OD) δ 7.94-7.87 (m, 1H), 7.75-7.60 (m, 3H), 6.77-6.67 (m, 1H), 5.40-5.20 (m, 1H), 4.41- 4.24 (m, 3H), 4.13-3.96 (m, 2H), 3.27-3.15 (m, 3H), 3.04-2.96 (m, 1H), 2.74-2.63 (m, 3H), 2.41-2.10 (m, 3H), 2.03-1.83 (m, 3H), 1.77-1.68 (m, 3H), 1.36-1.30 (m, 3H). |
| | 342 & 364 | | |
| 365 | 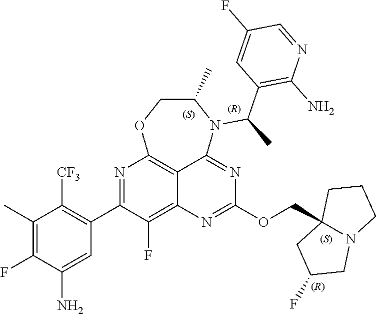 | 706.7 | (400 MHz, CD₃OD) δ 7.92-7.86 (m, 1H), 7.70-7.63 (m, 1H), 6.79-6.70 (m, 1H), 6.70-6.48 (m, 1H), 5.39- 5.19 (m, 1H), 4.65-4.56 (m, 1H), 4.37-4.23 (m, 3H), 4.13-4.03 (m, 1H), 3.27-3.10 (m, 3H), 3.06-2.95 (m, 1H), 2.41-2.09 (m, 6H), 2.06-1.83 (m, 3H), 1.70-1.61 (m, 3H), 0.74-0.67 (m, 3H). |
| | + | | |
| 366 | 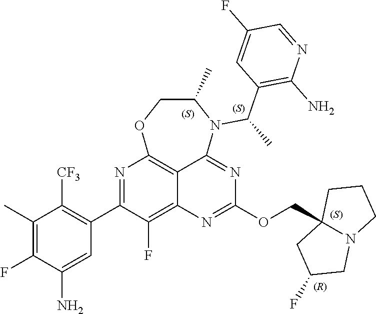 | 706.7 | (400 MHz, CD₃OD) δ 7.94-7.84 (m, 1H), 7.71-7.65 (m, 1H), 6.75-6.51 (m, 2H), 5.38-5.21 (m, 1H), 4.38- 4.30 (m, 2H), 4.27-4.22 (m, 1H), 4.06-3.94 (m, 2H), 3.28-3.11 (m, 3H), 3.04-2.96 (m, 1H), 2.39-2.36 (m, 3H), 2.35-2.08 (m, 3H), 2.03-1.84 (m, 3H), 1.74-1.67 (m, 3H), 1.28-1.25 (m, 3H). |
| | 365 & 366 | | |
| 387 | 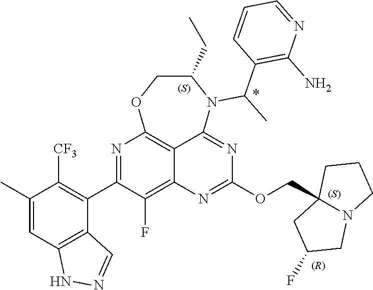 | 709.7 | (400 MHz, CD₃OD) δ 8.01-7.92 (m, 1H), 7.86-7.78 (m, 1H), 7.75-7.56 (m, 2H), 6.83-6.72 (m, 2H), 5.41- 5.18 (m, 1H), 4.85-4.73 (m, 1H), 4.40-4.23 (m, 3H), 3.86-3.74 (m, 1H), 3.27-3.13 (m, 3H), 3.05-2.92 (m, 1H), 2.73-2.61 (m, 3H), 2.39-2.08 (m, 3H), 2.06-1.81 (m, 3H), 1.75-1.59 (m, 3H), 1.59-1.39 (m, 1H), 0.72- 0.59 (m, 4H). |
| 388 | 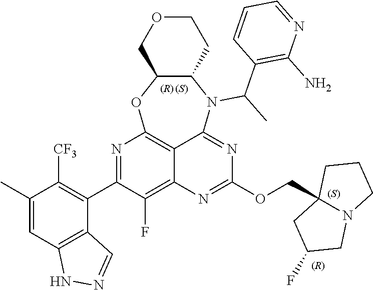 | 737.7 | (400 MHz, CD₃OD) δ 8.04-7.57 (m, 4H), 6.81-6.71 (m, 1H), 5.51-5.43 (m, 1H), 5.40-5.20 (m, 1H), 4.56- 4.45 (m, 1H), 4.32-4.16 (m, 3H), 3.84-3.75 (m, 1H), 3.64-3.55 (m, 1H), 3.36-3.30 (m, 1H), 3.27-3.16 (m, 3H), 3.09-2.96 (m, 2H), 2.71-2.64 (m, 3H), 2.36-2.14 (m, 3H), 2.06-1.94 (m, 6H), 1.88-1.68 (m, 2H). |
| 389 | 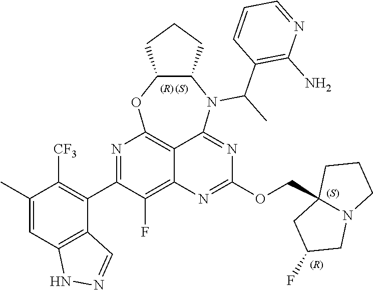 | 721.7 | (400 MHz, CD₃OD) δ 8.03-7.92 (m, 1H), 7.85-7.79 (m, 1H), 7.74-7.58 (m, 2H), 6.83-6.73 (m, 1H), 6.70- 6.32 (m, 1H), 5.42-5.19 (m, 1H), 5.04-4.61 (m, 1H), 4.39-4.27 (m, 2H), 3.99-3.67 (m, 1H), 3.25-3.17 (m, 2H), 3.06-2.97 (m, 1H), 2.71-2.64 (m, 3H), 2.41-1.37 (m, 16H). |
| 390 | 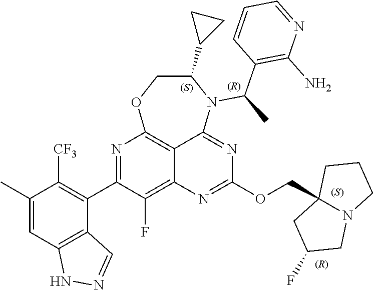 | 721.7 | (400 MHz, CD₃OD) δ 8.01-7.89 (m, 1H), 7.77-7.51 (m, 3H), 6.90-6.66 (m, 2H), 5.39-5.16 (m, 1H), 4.79- 4.68 (m, 1H), 4.42-4.26 (m, 3H), 3.28-3.10 (m, 4H), 3.08-2.89 (m, 1H), 2.80-2.55 (m, 3H), 2.41-2.06 (m, 3H), 2.07-1.77 (m, 3H), 1.62 (d, J = 6.9 Hz, 3H), 0.92-0.55 (m, 1H), 0.52-0.29 (m, 1H), 0.2-−0.19 (m, 2H), −1.08-−1.31 (m, 1H). |
| 391 | 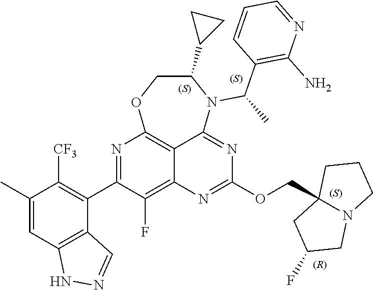 | 721.7 | (400 MHz, CD₃OD) δ 7.97 (d, J = 5.1 Hz, 1H), 7.89- 7.48 (m, 3H), 6.96-6.47 (m, 2H), 5.42-5.20 (m, 1H), 4.50-4.39 (m, 1H), 4.39-4.23 (m, 2H), 4.22-4.07 (m, 1H), 3.27-3.11 (m, 3H), 3.06-2.90 (m, 2H), 2.78-2.56 (m, 3H), 2.43-2.07 (m, 3H), 2.04-1.68 (m, 6H), 1.04- 0.64 (m, 3H), 0.54-0.33 (m, 2H). |
| 392 | 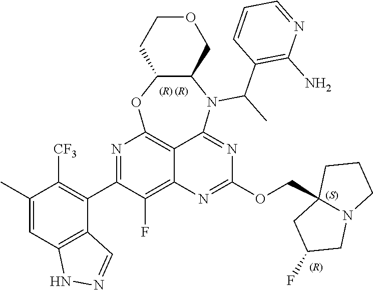 | 737.7 | (400 MHz, CD₃OD) δ 8.02-7.93 (m, 1H), 7.91-7.83 (m, 1H), 7.80-7.57 (m, 2H), 6.83-6.71 (m, 1H), 5.45- 5.15 (m, 2H), 4.68-4.58 (m, 1H), 4.34-4.20 (m, 3H), 3.94-3.83 (m, 1H), 3.52-3.43 (m, 1H), 3.40-3.33 (m, 1H), 3.27-3.15 (m, 4H), 3.06-2.96 (m, 1H), 2.72- 2.63 (m, 3H), 2.37-2.13 (m, 3H), 2.06-1.84 (m, 8H). |
| 393 | 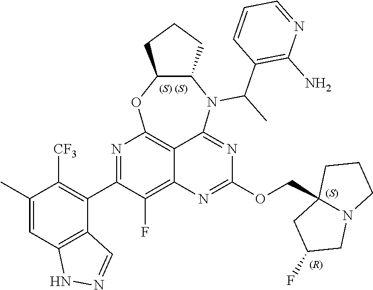 | 721.8 | (400 MHz, CD₃OD) δ 8.11-7.55 (m, 4H), 6.89-6.63 (m, 1H), 6.51-5.79 (m, 1H), 5.43-5.17 (m, 1H), 4.39- 4.15 (m, 2H), 3.74-3.57 (m, 1H), 3.27-3.10 (m, 3H), 3.09-2.93 (m, 1H), 276-2.58 (m, 3H), 2.40-2.02 (m, 5H), 1.99-1.83 (m, 7H), 1.79-1.49 (m, 4H). |
| 398 | 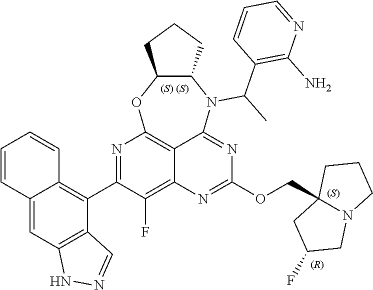 | 689.8 | (400 MHz, CD₃OD) δ 8.20-7.64 (m, 6H), 7.49-7.25 (m, 2H), 6.91-6.63 (m, 1H), 6.43-5.79 (m, 1H), 5.40- 5.16 (m, 1H), 4.32-4.17 (m, 2H), 3.69-3.52 (m, 1H), 3.28-3.11 (m, 3H), 3.07-2.89 (m, 1H), 2.35-1.55 (m, 16H). |
| 399 | 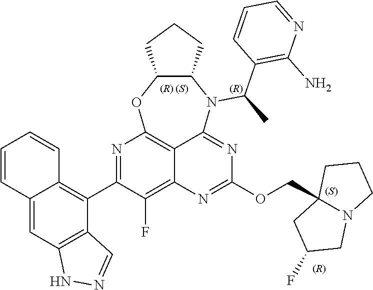 | 689.7 | (400 MHz, CD₃OD) δ 8.19-8.13 (m, 1H), 8.11-8.00 (m, 2H), 8.00-7.94 (m, 1H), 7.88-7.75 (m, 2H), 7.49- 7.42 (m, 1H), 7.38-7.30 (m, 1H), 6.80-6.74 (m, 1H), 6.71-6.62 (m, 1H), 5.42-5.22 (m, 1H), 5.00-4.95 (m, 1H), 4.42-4.31 (m, 2H), 3.97-3.87 (m, 1H), 3.36-3.31 (m, 1H), 3.25-3.22 (m, 1H), 3.09-3.00 (m, 1H), 2.44- 1.69 (m, 13H), 1.60-1.38 (m, 2H), 0.95-0.85 (m, 1H). |
| | + | | |
| 400 | 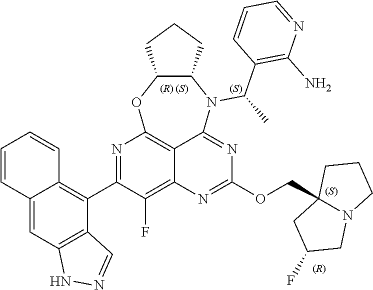 | 689.7 | (400 MHz, CD₃OD) δ 8.19-8.13 (m, 1H), 8.07-7.98 (m, 3H), 7.84-7.76 (m, 2H), 7.48-7.43 (m, 1H), 7.37- 7.32 (m, 1H), 6.84-6.77 (m, 1H), 6.50-6.41 (m, 1H), 5.41-5.23 (m, 1H), 4.70-4.65 (m, 1H), 4.42-4.32 (m, 2H), 3.81-3.71 (m, 1H), 3.39-3.32 (m, 1H), 3.26-3.22 (m, 1H), 3.08-3.00 (m, 1H), 2.44-1.96 (m, 8H), 1.95- 1.75 (m, 4H), 1.73-1.69 (m, 3H), 1.68-1.58 (m, 1H). |
| | 399 & 400 | | |

| Com- | | Catalyst, alkali and |
|---|---|---|
| pound | Intermediate | solvent used in step 3 |
| 4 | I-A3 | cataCXium A Pd-G₃, |
| | 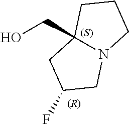 | K₃PO₄, MeCN, H₂O |
| | I-B1 | |
| | I-C3 | |
| 15 | I-A4 | cataCXium A Pd-G₃, |
| | I-B1 | K₃PO₄, THF, H₂O |
| | I-C3 | |
| 18 | I-A6 | cataCXium A Pd-G₃, |
| | I-B1 | K₃PO₄, THF, H₂O |
| | I-C3 | |
| 25 | I-A4 | cataCXium A Pd-G₃, |
| | I-B4 | K₃PO₄, THF, H₂O |
| | I-C3 | |
| 36 | I-A6 | cataCXium A Pd-G₃, |
| | 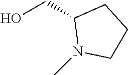 | K₃PO₄, THF, H₂O |
| | I-B3 | |
| | I-C3 | |
| 37 | I-A6 | cataCXium A Pd-G₃, |
| | 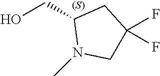 | K₃PO₄, THF, H₂O |
| | I-B8 | |
| | I-C3 | |
| 40 | I-A4 | cataCXium A Pd-G₃, |
| | I-B8 | K₃PO₄, DMA, H₂O |
| | I-C3 | |
| 59 | I-A12 | cataCXium A Pd-G₃, |
| | I-B1 | K₃PO₄, THF, H₂O |
| | I-C3 | |
| 60 | I-A13 | cataCXium A Pd-G₃, |
| | I-B1 | K₃PO₄, THF, H₂O |
| | I-C3 | |
| 63 | I-A14 | cataCXium A Pd-G₃, |
| | I-B1 | K₃PO₄, THF, H₂O |
| | I-C3 | |
| 64 | I-A14 | cataCXium A Pd-G₃, |
| | I-B3 | K₃PO₄, THF, H₂O |
| | I-C3 | |
| 66 | I-A15 | Pd(dppf)Cl₂•CH₂Cl₂, |
| | I-B1 | Cs₂CO₃, |
| | I-C3 | p-dioxane, H₂O |
| 162 | I-A27 | Pd(dppf)Cl₂•CH₂Cl₂, |
| | I-B1 | K₂CO₃, DMA, H₂O |
| | I-C9 | |
| 259 | 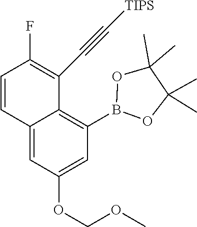 | cataCXium A Pd-G₃, K₃PO₄, DMA, H₂O |
| | I-A30 | |
| | I-B1 | |
| | I-C9 | |
| 314 | 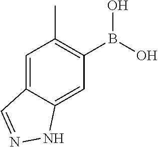 | RuPhos Pd G3, K₃PO₄, DMA, H₂O |
| | I-A47 | |
| | I-B1 | |
| | I-C29 | |
| 315 | I-A48 | RuPhos Pd G3, |
| | I-B1 | K₃PO₄, DMA, H₂O |
| | I-C29 | |
| 316 | 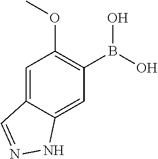 | RuPhos Pd G3, K₃PO₄, DMA, H₂O |
| | I-A49 | |
| | I-B1 | |
| | I-C29 | |
| 317 | I-A50 | RuPhos Pd G3, |
| | I-B1 | K₃PO₄, DMA, H₂O |
| | I-C29 | |
| 318 | 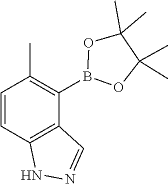 [SMILES: Cc1ccc2[nH]ncc2c1B1OC(C)(C)C(C)(C)O1] | RuPhos Pd G3, K₃PO₄, DMA, H₂O |
| | I-A51 | |
| | I-B1 | |
| | I-C29 | |
| 319 | 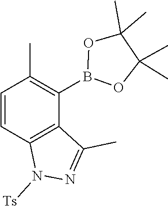 [SMILES: Cc1ccc(S(=O)(=O)n2nc(C)c3c(B4OC(C)(C)C(C)(C)O4)c(C)ccc32)cc1] | RuPhos Pd G3, K₃PO₄, DMA, H₂O |
| | I-A52 | |
| | I-B1 | |
| | I-C29 | |
| 320 | 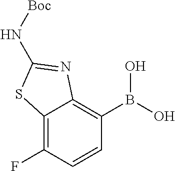 [SMILES: CC(C)(C)OC(=O)Nc1nc2c(B(O)O)ccc(F)c2s1] | RuPhos Pd G3, K₃PO₄, DMA, H₂O |
| | I-A7 | |
| | I-B1 | |
| | I-C29 | |
| 321 | I-A53 | RuPhos Pd G3, |
| | I-B1 | K₃PO₄, DMA, H₂O |
| | I-C29 | |
| 322 | I-A54 | RuPhos Pd G3, |
| | I-B1 | K₃PO₄, DMA, H₂O |
| | I-C29 | |
| 323 | I-A55 | RuPhos Pd G3, |
| | I-B1 | K₃PO₄, DMA, H₂O |
| | I-C29 | |
| 324 | I-A56 | RuPhos Pd G3, |
| | I-B1 | K₃PO₄, DMA, H₂O |
| | I-C29 | |
| 325 | I-A57 | RuPhos Pd G3, |
| | I-B1 | K₃PO₄, DMA, H₂O |
| | I-C29 | |
| 327 | 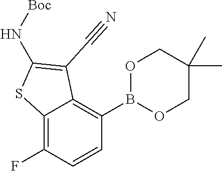 [SMILES: CC1(C)COB(c2ccc(F)c3sc(NC(=O)OC(C)(C)C)c(C#N)c23)OC1] | Pd(PPh₃)₂Cl₂, K₂CO₃, PhMe |
| | I-A59 | |
| | I-B1 | |
| | I-C29 | |
| 328 | 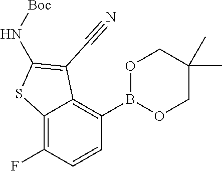 [SMILES: CC1(C)COB(c2ccc(F)c3sc(NC(=O)OC(C)(C)C)c(C#N)c23)OC1] | Pd(PPh₃)₂Cl₂, K₂CO₃, PhMe |
| | I-A59 | |
| | I-B1 | |
| | I-C29 | |
| 332 | I-A63 | RuPhos Pd G3, |
| | I-B1 | K₃PO₄, THF, H₂O |
| | I-C29 | |
| 333 | 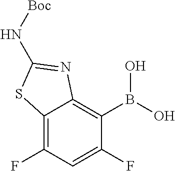 [SMILES: CC(C)(C)OC(=O)Nc1nc2c(B(O)O)c(F)cc(F)c2s1] | RuPhos Pd G3, K₃PO₄, THF, H₂O |
| | I-A64 | |
| | I-B1 | |
| | I-C29 | |
| 337 | I-A67 | Pd(dppf)Cl₂, K₃PO₄, |
| | I-B1 | THF, H₂O |
| | I-C29 | |
| 360 | I-A70 | RuPhos Pd G3, |
| | I-B1 | K₃PO₄, THF, H₂O |
| | I-C29 | |
| 342 | I-A45 | RuPhos Pd G3, |
| | I-B1 | K₃PO₄, THF, H₂O |
| | I-C33 | Chiral HPLC |
| | | resolution conditions: |
| | | column: ODH (2 × 25 |
| | | cm); mobile phase: n- |
| | | heptane/ethanol (0.1% |
| | | ammonia water) = |
| | | 70:30. |
| 364 | I-A45 | RuPhos Pd G3, |
| | I-B1 | K₃PO₄, THF, H₂O |
| | I-C33 | Chiral HPLC |
| | | resolution conditions: |
| | | column: ODH (2 × 25 |
| | | cm); mobile phase: n- |
| | | heptane/ethanol (0.1% |
| | | ammonia water) = |
| | | 70:30. |
| 365 | I-A18 | Pd(dppf)Cl₂, K₃PO₄, |
| | I-B1 | THF, H₂O |
| | I-C33 | Chiral HPLC |
| | | resolution conditions: |
| | | column: ODH (2 × 25 |
| | | cm); mobile phase: n- |
| | | heptane/ethanol (0.1% |
| | | ammonia water) = |
| | | 70:30. |
| 366 | I-A18 | Pd(dppf)Cl₂, K₃PO₄, |
| | I-B1 | THF, H₂O |
| | I-C33 | Chiral HPLC |
| | | resolution conditions: |
| | | column: ODH (2 × 25 |
| | | cm); mobile phase: n- |
| | | heptane/ethanol (0.1% |
| | | ammonia water) = |
| | | 70:30. |
| 387 | I-A45 | RuPhos Pd G3, |
| | I-B1 | K₃PO₄, THF, H₂O |
| | I-C35 | |
| 388 | I-A45 | RuPhos Pd G3, |
| | I-B1 | K₃PO₄, THF, H₂O |
| | I-C36 | |
| 389 | I-A45 | RuPhos Pd G3, |
| | I-B1 | K₃PO₄, THF, H₂O |
| | I-C37 | |
| 390 | I-A45 | RuPhos Pd G3, |
| | I-B1 | K₃PO₄, THF, H₂O |
| | I-C38 | |
| 391 | I-A45 | RuPhos Pd G3, |
| | I-B1 | K₃PO₄, THF, H₂O |
| | I-C39 | |
| 392 | I-A45 | RuPhos Pd G3, |
| | I-B1 | K₃PO₄, THF, H₂O |
| | I-C40 | |
| 393 | I-A45 | RuPhos Pd G3, |
| | I-B1 | K₃PO₄, THF, H₂O |
| | I-C41 | |
| 398 | 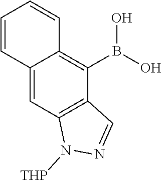 | RuPhos Pd G3, K₃PO₄, THF, H₂O |
| | I-A85 | |
| | I-B1 | |
| | I-C41 | |
| 399 | 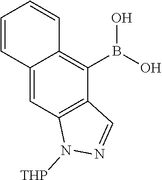 | RuPhos Pd G3, K₃PO₄, THF, H₂O Thin-layer chromatography separation conditions: DCM/MeOH/NH₃H₂O aq. = 480/30/5, Rf = 0.60 |
| | I-A85 | |
| | I-B1 | |
| | I-C37 | |
| 400 | 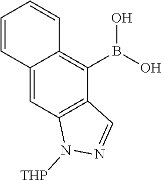 | RuPhos Pd G3, K₃PO₄, THF, H₂O Thin-layer chromatography separation conditions: DCM/MeOH/NH₃H₂O aq. = 480/30/5, Rf = 0.55 |
| | I-A85 | |
| | I-B1 | |
| | I-C37 | |

Note:
intermediates I-A7, I-A30, I-A47, I-A49, I-A51, I-A52, I-A59, I-A64, I-A85, I-B1, I-B3 and I-B8 were all commercially available.

The diastereomers in the table were subjected to chiral HPLC analysis under the following conditions (flow rate: 1 mnL/minute; detector: UV 254 nm):

| Compound | Column | Mobile phase | RT/minute | de % |
|---|---|---|---|---|
| 342 | ODH | n-heptane/ | 6.855 | 100% |
| 364 | (0.46 × 25 cm) | ethanol (0.1% | 5.132 | 100% |
| | | ammonia | | |
| | | water) = 70:30 | | |
| 365 | ODH | n-heptane/ | 7.628 | 100% |
| 366 | (0.46 × 25 cm) | ethanol (0.1% | 5.459 | 100% |
| | | ammonia | | |
| | | water) = 70:30 | | |

### Compounds 2 and 3 3-(1-(5-(5,6-Dimethyl-1H-indazol-4-yl)-4-fluoro-2-(((2R,7aS)-2-fluorotetrahydro-1H-pyrrolizin-7a(5H)-yl)methoxy)-8,9-dihydro-1OH-7-oxa-1,3,6,10-tetraazacyclohepta[de]naphthalen-10-yl)ethyl)pyridin-2-amine and 10-(1-(2-Aminopyridin-3-yl)ethyl)-5-(5,6-dimethyl-1H-indazol-4-yl)-4-fluoro-9,10-dihydro-8H-7-oxa-1,3,6,10-tetraazacyclohepta[de]naphthalen-2-ol

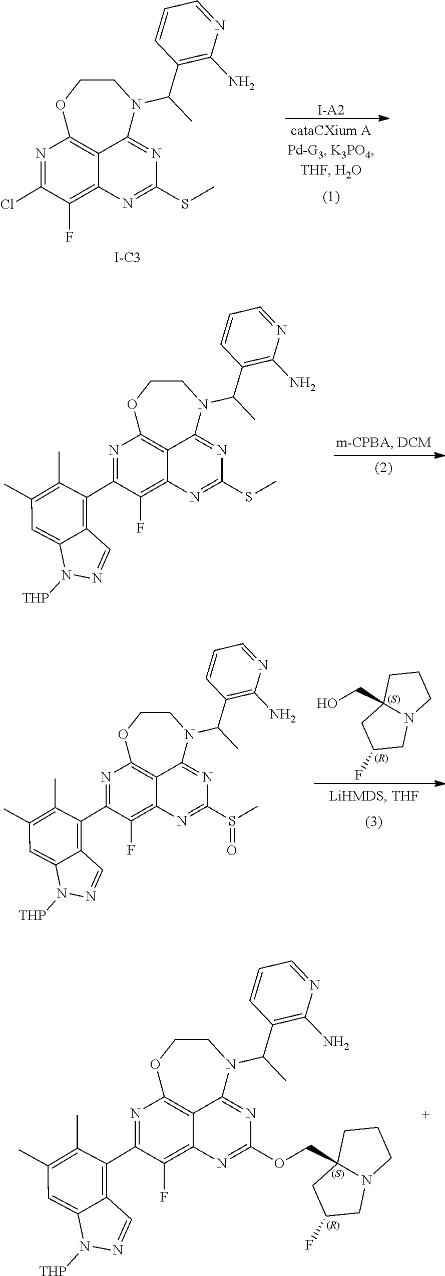

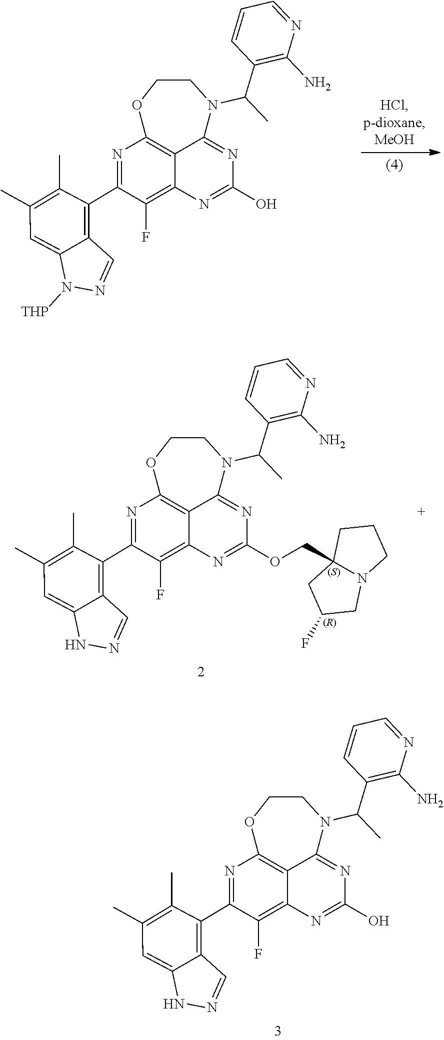

### Step 1: 3-(1-(5-(5,6-dimethyl-1-(tetrahydro-2H-pyran-2-yl)-1H-indazol-4-yl)-4-fluoro-2-(methylthio)-8,9-dihydro-1OH-7-oxa-1,3,6,10-tetraazacyclohepta[de]naphthalen-10-yl)ethyl)pyridin-2-amine

The intermediate I-C3 (186 mg, 0.46 mmol), the intermediate I-A2 (328 mg, 0.92 mmol), cataCXium A Pd—G₃ (67 mg, 0.092 mmol) and anhydrous potassium phosphate (293 mg, 1.38 mmol) were dissolved in tetrahydrofuran/water (5 mL/1 mL) and stirred at 70° C. for 3 hours under nitrogen protection. The reaction solution was concentrated in vacuum under reduced pressure, and the resulting residue was purified by column chromatography on silica gel (water/methanol) to give the target product (205 mg, yield 74%) as a yellow solid. [M+H]⁺ 601.4

### Step 2: 3-(1-(5-(5,6-dimethyl-1-(tetrahydro-2H-pyran-2-yl)-1H-indazol-4-yl)-4-fluoro-2-(methylsulfinyl)-8,9-dihydro-1OH-7-oxa-1,3,6,10-tetraazacyclohepta[de]naphthalen-10-yl)ethyl)pyridin-2-amine

At 0-5° C., m-CPBA (at a content of 85%, 37 mg, 0.18 mmol) was added to a solution of 3-(1-(5-(5,6-dimethyl-1-(tetrahydro-2H-pyran-2-yl)-1H-indazol-4-yl)-4-fluoro-2-(methylthio)-8,9-dihydro-1OH-7-oxa-1,3,6,10-tetraazacyclohepta[de]naphthalen-10-yl)ethyl)pyridin-2-amine (95 mg, 0.16 mmol) in dichloromethane (10 mL) and stirred for 30 minutes. Water was added to the reaction solution and extracted with dichloromethane (5 mL×3). The organic phase was collected and concentrated in vacuum under reduced pressure to give a crude target product (99 mg), which was used in the reaction of the next step directly. [M+H]⁺ 617.3

### Step 3: 3-(1-(5-(5,6-dimethyl-1-(tetrahydro-2H-pyran-2-yl)-1H-indazol-4-yl)-4-fluoro-2-(((2R,7aS)-2-fluorotetrahydro-1H-pyrrolizin-7a(5H)-yl)methoxy)-8,9-dihydro-1OH-7-oxa-1,3,6,10-tetraazacyclohepta[de]naphthalen-10-yl)ethyl)pyridin-2-amine and 10-(1-(2-aminopyridin-3-yl)ethyl)-5-(5,6-dimethyl-1-(tetrahydro-2H-pyran-2-yl)-1H-indazol-4-yl)-4-fluoro-9,10-dihydro-8H-7-oxa-1,3,6,10-tetraazacyclohepta[de]naphthalen-2-ol

At 0-5° C., a 1.3 M LiHMDS/THF solution (0.34 mL, 0.44 mmoL) was added dropwise to a solution of 3-(1-(5-(5,6-dimethyl-1-(tetrahydro-2H-pyran-2-yl)-1H-indazol-4-yl)-4-fluoro-2-(methylsulfinyl)-8,9-dihydro-1OH-7-oxa-1,3,6,10-tetraazacyclohepta[de]naphthalen-10-yl)ethyl)pyridin-2-amine (92 mg, 0.15 mmoL) and the intermediate I-B1 (72 mg, 0.45 mmoL) in anhydrous tetrahydrofuran (5 mL) and stirred for 30 minutes. Water was added to the reaction solution and extracted with dichloromethane (5 mL×3). The organic phase was collected and concentrated in vacuum under reduced pressure, and the resulting residue was purified by column chromatography on silica gel (dichloromethane/methanol) to give a mixture of crude target products as a pale yellow solid, which was used in the reaction of the next step directly.

### Step 4: 3-(1-(5-(5,6-dimethyl-1H-indazol-4-yl)-4-fluoro-2-(((2R,7aS)-2-fluorotetrahydro-1H-pyrrolizin-7a(5H)-yl)methoxy)-8,9-dihydro-1OH-7-oxa-1,3,6,10-tetraazacyclohepta[de]naphthalen-10-yl)ethyl)pyridin-2-amine and 10-(1-(2-aminopyridin-3-yl)ethyl)-5-(5,6-dimethyl-1H-indazol-4-yl)-4-fluoro-9,10-dihydro-8H-7-oxa-1,3,6,10-tetraazacyclohepta[de]naphthalen-2-ol

At room temperature, a 4 M hydrogen chloride/1,4-dioxane solution (4 mL) was added to a solution of the mixture (15 mg) obtained in step 3 in methanol (0.5 mL) and stirred for 30 minutes. After concentration in vacuum under reduced pressure, water (1 mL) and concentrated ammonia water (0.5 mL) were added to the resulting residue and extracted with dichloromethane, and the organic phase was collected and concentrated in vacuum under reduced pressure. The resulting residue was purified by column chromatography on silica gel (dichloromethane/methanol) to give the compound 2 (12 mg) and the compound 3 (15 mg) as pale yellow solids.

Compound 2, [M+H]⁺ 628.3. ¹H NMR (400 MHz, CD₃OD) δ 7.99-7.94 (m, 1H), 7.78-7.74 (m, 1H), 7.63 (s, 1H), 7.47 (s, 1H), 6.80-6.76 (m, 1H), 6.66-6.58 (mz, 1H), 5.41-5.24 (m, 1H), 4.52-4.46 (m, 1H), 4.40-4.32 (m, 3H), 3.86-3.73 (m, 1H), 3.64-3.58 (m, 1H), 3.24-3.19 (m, 3H), 3.07-3.00 (m, 1H), 2.48 (s, 3H), 2.36-2.26 (m, 1H), 2.22 (s, 3H), 2.20-2.13 (m, 2H), 2.02-1.92 (m, 3H), 1.68-1.64 (m, 3H)

Compound 3, [M+H]⁺ 487.2. ¹H NMR (400 MHz, CD₃OD) δ 7.96-7.92 (m, 1H), 7.70-7.63 (m, 2H), 7.44 (s, 1H), 6.78-6.69 (m, 2H), 4.50-4.20 (m, 3H), 3.76-3.50 (m, 2H), 2.44 (s, 3H), 2.22 (s, 3H), 1.63-1.59 (m, 3H)

The compounds in the table below were prepared from corresponding intermediates and reagents by following the preparation steps of the compounds 2 and 3:

| | | LC- | |
|---|---|---|---|
| | | MS | |
| Com- | | [M + | |
| pound | Structural formula | H]⁺ | ¹HNMR |
| 5 | 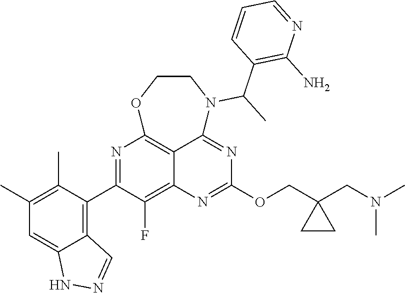 | 598.4 | (400 MHz, CD₃OD) δ 8.28-8.22 (m, 1H), 8.01-7.96 (m, 1H), 7.66 (s, 1H), 7.51 (s, 1H), 7.11-7.05 (m, 1H), 6.61 (s, 1H), 4.66-4.44 (m, 4H), 4.11-3.85 (m, 1H), 3.79-3.66 (m, 1H), 3.41-3.31 (m, 2H), 3.01 (s, 6H), 2.49 (s, 3H), 2.24 (s, 3H), 1.75 (s, 3H), 1.03- 0.96 (m, 2H), 0.92-0.88 (m, 2H). |
| 6 | 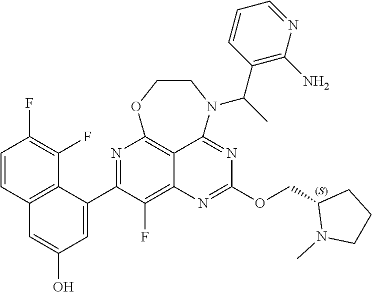 | 618.3 | (400 MHz, CD₃OD) δ 7.99-7.92 (m, 1H), 7.79-7.75 (m, 1H), 7.61-7.56 (m, 1H), 7.42-7.34 (m, 1H), 7.31- 7.27 (m, 1H), 7.22-7.17 (m, 1H), 6.81-6.76 (m, 1H), 6.67-6.59 (m, 1H), 4.61-4.29 (m, 4H), 3.83-3.72 (m, 1H), 3.61-3.50 (m, 1H), 3.20-3.11 (m, 1H), 3.00-2.87 (m, 1H), 2.58 (s, 3H), 2.52-2.39 (m, 1H), 2.20-2.10 (m, 1H), 1.91-1.77 (m, 3H), 1.70-1.63 (m, 3H). |
| 7 | 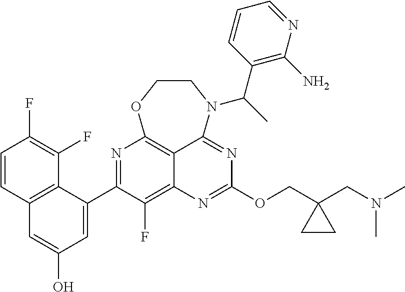 [SMILES: CC(c1cccnc1N)N1CCOc2nc(-c3cc(O)cc4ccc(F)c(F)c34)c(F)c3nc(OCC4(CN(C)C)CC4)nc1c23] | 632.3 | (400 MHz, CD₃OD) δ 8.02-7.90 (m, 1H), 7.80-7.72 (m, 1H), 7.62-7.55 (m, 1H), 7.42-7.34 (m, 1H), 7.32- 7.27 (m, 1H), 7.23-7.17 (m, 1H), 6.84-6.73 (m, 1H), 6.67-6.57 (m, 1H), 4.53-4.29 (m, 4H), 3.83-3.71 (m, 1H), 3.63-3.52 (m, 1H), 2.78-2.57 (m, 2H), 2.49 (s, 6H), 1.72-1.61 (m, 3H), 0.85-0.76 (m, 2H), 0.68-0.55 (m, 2H). |
| 8 | 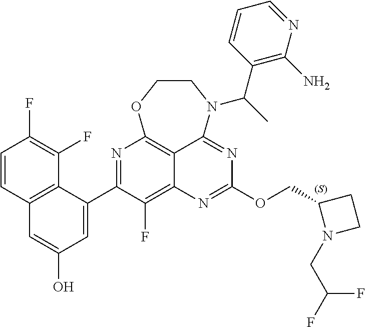 | 654.2 | (400 MHz, CD₃OD) δ 7.99-7.92 (m, 1H), 7.80-7.75 (m, 1H), 7.61-7.56 (m, 1H), 7.42-7.34 (m, 1H), 7.31- 7.28 (m, 1H), 7.22-7.17 (m, 1H), 6.82-6.77 (m, 1H), 6.67-6.59 (m, 1H), 6.08-5.66 (m, 1H), 4.65-4.29 (m, 4H), 3.86-3.70 (m, 2H), 3.64-3.48 (m, 2H), 3.27-3.11 (m, 2H), 2.91-2.74 (m, 1H), 2.26-2.11 (m, 2H), 1.72- 1.62 (m, 3H). |
| 9 | 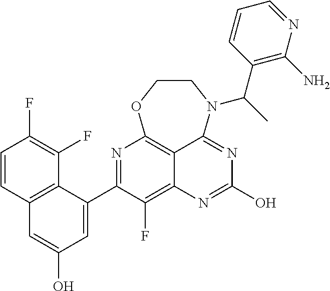 [SMILES: CC(c1cccnc1N)N1CCOc2nc(-c3cc(O)cc4ccc(F)c(F)c34)c(F)c3nc(O)nc1c23] | 521.2 | (400 MHz, CD₃OD) δ 8.02-7.96 (m, 1H), 7.96-7.91 (m, 1H), 7.63-7.58 (m, 1H), 7.44-7.37 (m, 1H), 7.33- 7.30 (m, 1H), 7.24-7.19 (m, 1H), 6.92-6.86 (m, 1H), 6.71-6.64 (m, 1H), 4.58-4.49 (m, 1H), 4.42-4.26 (m, 1H), 3.88-3.80 (m, 1H), 3.65-3.54 (m, 1H), 1.7-1.65 (m, 3H). |
| 10 | 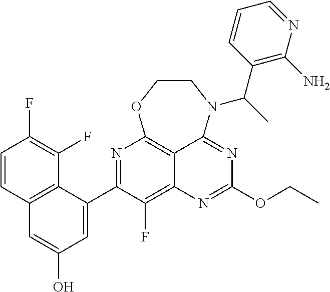 [SMILES: CCOc1nc2c3c(nc(-c4cc(O)cc5ccc(F)c(F)c45)c(F)c3n1)OCCN2C(C)c1cccnc1N] | 549.2 | (400 MHz, CD₃OD) δ 7.98-7.93 (m, 1H), 7.84-7.79 (m, 1H), 7.61-7.56 (m, 1H), 7.42-7.34 (m, 1H), 7.32- 7.27 (m, 1H), 7.23-7.16 (m, 1H), 6.84-6.79 (m, 1H), 6.68-6.59 (m, 1H), 4.62-4.30 (m, 4H), 3.82-3.73 (m, 1H), 3.62-3.51 (m, 1H), 1.70-1.64 (m, 3H), 1.51-1.40 (m, 3H). |
| 11 | 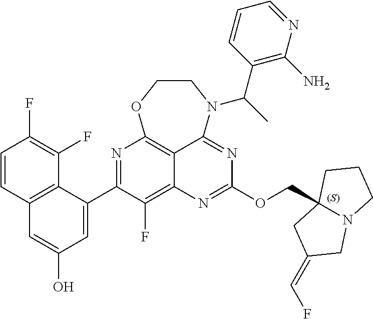 | 674.3 | (400 MHz, CD₃OD) δ 7.98-7.93 (m, 1H), 7.79-7.74 (m, 1H), 7.62-7.56 (m, 1H), 7.42-7.34 (m, 1H), 7.31- 7.27 (m, 1H), 7.22-7.17 (m, 1H), 6.81-6.53 (m, 3H), 4.53-4.28 (m, 4H), 3.89-3.73 (m, 2H), 3.58-3.44 (m, 2H), 3.22-3.11 (m, 1H), 2.82-2.66 (m, 2H), 2.51-2.42 (m, 1H), 2.21-2.12 (m, 1H), 2.04-1.84 (m, 3H), 1.66 (d, J = 6.7 Hz, 3H). |
| 12 | 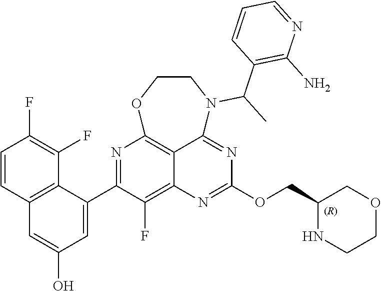 | 620.3 | (400 MHz, CD₃OD) δ 7.99-7.93 (m, 1H), 7.79-7.74 (m, 1H), 7.61-7.56 (m, 1H), 7.42-7.34 (m, 1H), 7.31- 7.28 (m, 1H), 7.22-7.18 (m, 1H), 6.82-6.76 (m, 1H), 6.66-6.58 (m, 1H), 4.55-4.28 (m, 4H), 4.00-3.92 (m, 1H), 3.82-3.73 (m, 2H), 3.65-3.39 (m, 4H), 2.97-2.89 (m, 2H), 1.67 (d, J = 6.7 Hz, 3H). |
| 13 | 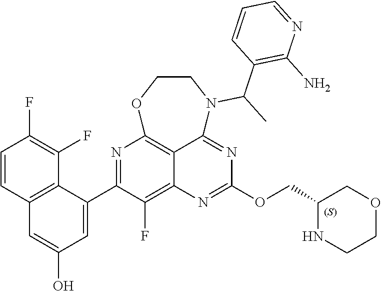 | 620.3 | (400 MHz, CD₃OD) δ 7.98-7.94 (m, 1H), 7.79-7.74 (m, 1H), 7.61-7.56 (m, 1H), 7.42-7.34 (m, 1H), 7.31- 7.28 (m, 1H), 7.22-7.18 (m, 1H), 6.81-6.77 (m, 1H), 6.66-6.58 (m, 1H), 4.54-4.27 (m, 4H), 3.99-3.93 (m, 1H), 3.82-3.73 (m, 2H), 3.64-3.41 (m, 4H), 2.96-2.90 (m, 2H), 1.67 (d, J = 6.3 Hz, 3H). |
| 14 | 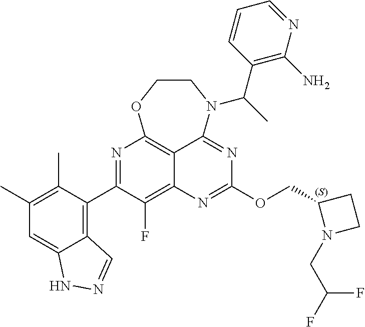 | 620.4 | (400 MHz, CD₃OD) δ 7.96 (d, J = 2.6 Hz, 1H), 7.77 (d, J = 7.4 Hz, 1H), 7.64 (s, 1H), 7.47 (s, 1H), 6.84- 6.75 (m, 1H), 6.64 (q, J = 7.0 Hz, 1H), 6.05-5.70 (m, 1H), 4.64-4.35 (m, 4H), 3.84-3.74 (m, 2H), 3.62-3.49 (m, 2H), 3.27-3.10 (m, 2H), 2.90-2.76 (m, 1H), 2.48 (s, 3H), 2.24-2.17 (m, 5H), 1.69-1.65 (d, J = 6.5 Hz, 3H). |
| 16 | 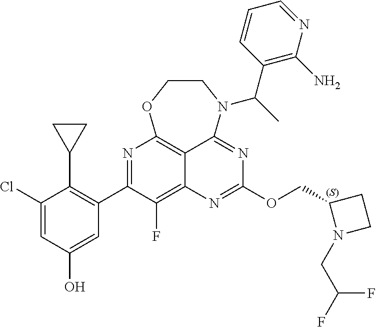 | 642.2 | 1H NMR (400 MHz, CD₃OD) δ 8.00-7.92 (m, 1H), 7.80-7.73 (m, 1H), 6.90 (d, J = 2.6 Hz, 1H), 6.82- 6.76 (m, 1H), 6.71 (d, J = 2.6 Hz, 1H), 6.66-6.57 (m, 1H), 6.05-5.70 (m, 1H), 4.62-4.27 (m, 4H), 4.65-4.27 (m, 2H), 3.60-3.49 (m, 2H), 3.28-3.08 (m, 2H), 2.91- 2.75 (m, 1H), 2.22-2.14 (m, 2H), 1.88-1.80 (m, 1H), 1.71-1.61 (m, 3H), 0.70-0.57 (m, 2H), 0.25-0.04 (m, 2H). |
| 17 | 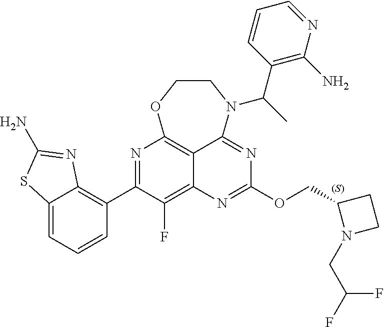 | 624.2 | (400 MHz, CD₃OD) δ 8.01-7.90 (m, 1H), 7.78-7.70 (m, 2H), 7.43-7.34 (m, 1H), 7.22-7.17 (m, 1H), 6.84- 6.73 (m, 1H), 6.67-6.54 (m, 1H), 6.05-5.68 (m, 1H), 4.56-4.52 (m, 1H), 4.49-4.42 (m, 1H), 4.40-4.25 (m, 1H), 3.83-3.71 (m, 2H), 3.64-3.62 (m, 1H), 3.57-3.49 (m, 2H), 3.28-3.17 (m, 1H), 3.17-3.10 (m, 1H), 2.89- 2.73 (m, 1H), 2.23-2.14 (m, 2H), 1.69-1.64 (m, 3H). |
| 19 | 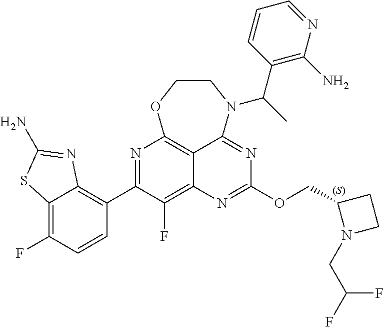 | 642.3 | (400 MHz, CD₃OD) δ 8.00-7.90 (m, 1H), 7.80-7.73 (m, 1H), 7.46-7.36 (m, 1H), 7.02-6.93 (m, 1H), 6.84- 6.74 (m, 1H), 6.66-6.55 (m, 1H), 6.09-5.69 (m, 1H), 4.63-3.99 (m, 4H), 3.87-3.70 (m, 2H), 3.57-3.48 (m, 2H), 3.26-3.08 (m, 2H), 2.92-2.74 (m, 1H), 2.25-2.10 (m, 2H), 1.71-1.61 (m, 3H). |
| 22 | 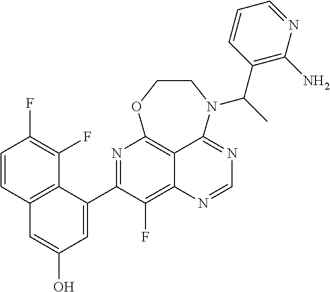 [SMILES: CC(c1cccnc1N)N1CCOc2nc(-c3cc(O)cc4ccc(F)c(F)c34)c(F)c3ncnc1c23] | 505.2 | (400 MHz, CD₃OD) δ 8.70 (s, 1H), 7.93 (d, J = 4.9 Hz, 1H), 7.74 (d, J = 7.5 Hz, 1H), 7.60-7.56 (m, 1H), 7.39-7.33 (m, 1H), 7.31-7.26 (m, 1H), 7.25-7.14 (m, 1H), 6.80-7.72 (m, 2H), 4.54-4.25 (m, 2H), 3.92-3.65 (m, 1H), 3.63-3.47 (m, 1H), 1.68-1.53 (m, 3H). |
| 33 | 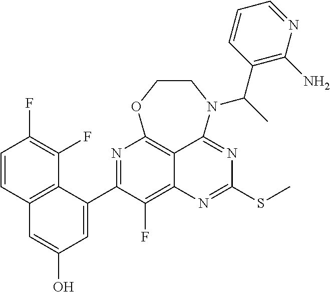 [SMILES: CSc1nc2c3c(nc(-c4cc(O)cc5ccc(F)c(F)c45)c(F)c3n1)OCCN2C(C)c1cccnc1N] | 551.2 | (400 MHz, CD₃OD) δ 7.97-7.91 (m, 1H), 7.78-7.72 (m, 1H), 7.61-7.54 (m, 1H), 7.42-7.32 (m, 1H), 7.31- 7.26 (m, 1H), 7.22-7.16 (m, 1H), 6.80-6.74 (m, 1H), 6.73-6.65 (m, 1H), 4.52-4.41 (m, 1H), 4.40-4.26 (m, 1H), 3.81-3.70 (m, 1H), 3.60-3.47 (m, 1H), 2.65 (s, 3H), 1.69-1.60 (m, 3H). |
| 45 | 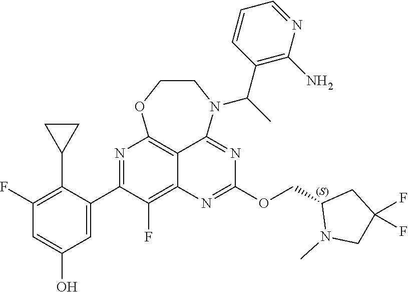 | 626.3 | (400 MHz, CD₃OD) δ 7.96-7.92 (m, 1H), 7.77-7.73 (m, 1H), 6.79-6.75 (m, 1H), 6.64-6.51 (m, 3H), 4.66- 4.50 (m, 2H), 4.48-4.40 (m, 1H), 4.37-4.28 (m, 1H), 3.79-3.69 (m, 1H), 3.57-3.48 (m, 1H), 3.43-3.34 (m, 1H), 3.13-3.01 (m, 1H), 2.81-2.67 (m, 1H), 2.67-2.62 (m, 1H), 2.62-2.50 (m, 1H), 2.50-2.44 (m, 3H), 2.39- 2.23 (m, 1H), 1.75-1.67 (m, 1H), 1.67-1.61 (m, 3H), 0.63-0.52 (m, 2H), 0.30-0.17 (m, 2H). |
| 46 | 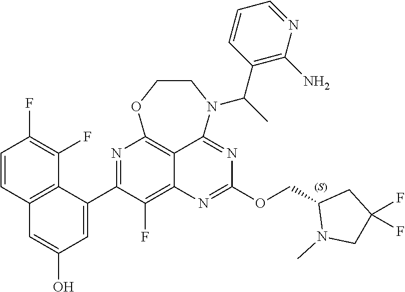 | 654.3 | (400 MHz, CD₃OD) δ 7.96-7.92 (m, 1H), 7.77-7.72 (m, 1H), 7.59-7.55 (m, 1H), 7.40-7.33 (m, 1H), 7.30- 7.26 (m, 1H), 7.21-7.18 (m, 1H), 6.79-6.74 (m, 1H), 6.65-6.57 (m, 1H), 4.67-4.52 (m, 2H), 4.50-4.28 (m, 2H), 3.80-3.70 (m, 1H), 3.58-3.48 (m, 1H), 3.42-3.33 (m, 1H), 3.13-3.02 (m, 1H), 2.80-2.66 (m, 1H), 2.66- 2.62 (m, 1H), 2.62-2.50 (m, 1H), 2.48 (s, 3H), 2.39- 2.23 (m, 1H), 1.67-1.62 (m, 3H). |
| 47 | 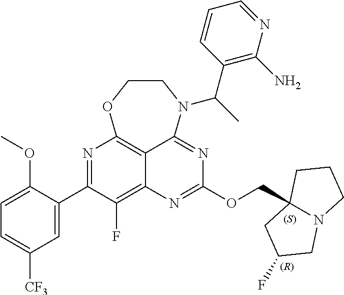 | 658.2 | (400 MHz, CD₃OD) δ 8.00-7.87 (m, 1H), 7.83-7.65 (m, 3H), 7.28 (d, J = 8.8 Hz, 1H), 6.77-6.71 (m, 1H), 6.56 (q, J = 6.5 Hz, 1H), 5.42-5.16 (m, 1H), 4.46- 4.42 (m, 1H), 4.39-4.17 (m, 3H), 3.87 (s, 3H), 3.80- 3.67 (m, 1H), 3.54-3.44 (m, 1H), 3.28-3.15 (m, 3H), 3.09-2.92 (m, 1H), 2.43-2.07 (m, 3H), 2.04-1.78 (m, 3H), 1.66-1.52 (m, 3H). |
| 65 | 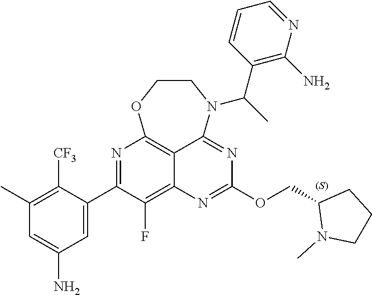 | 613.3 | (400 MHz, CD₃OD) δ 7.97-7.91(m, 1H), 7.78-7.70 (m, 1H), 6.79-6.74 (m, 1H), 6.69-6.65 (m, 1H), 6.63- 6.56 (m, 1H), 6.42-6.36 (m, 1H), 4.59-4.26 (m, 4H), 3.77-3.46 (m, 2H), 3.19-3.08 (m, 1H), 2.96-2.83 (m, 1H), 2.55 (s, 3H), 2.47-2.36 (m, 4H), 2.20-1.98 (m, 2H), 1.91-1.77 (m, 2H), 1.68-1.61 (m, 3H). |
| 67 | 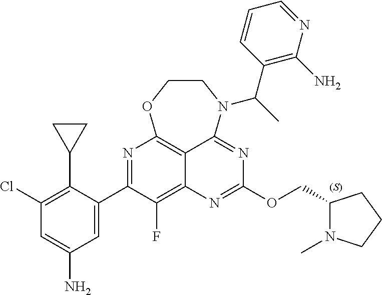 | 605.3 | (400 MHz, CD₃OD) δ 7.98-7.92 (m, 1H), 7.78-7.72 (m, 1H), 6.84-6.74 (m, 2H), 6.66-6.54 (m, 2H), 4.58- 4.41 (m, 3H), 4.36-4.27 (m, 1H), 3.78-3.67 (m, 1H), 3.57-3.48 (m, 1H), 3.16-3.07 (m, 1H), 2.92-2.80 (m, 1H), 2.54 (s, 3H), 2.46-2.35 (m, 1H), 2.21-1.97 (m, 2H), 1.89-1.74 (m, 4H), 1.69-1.61 (m, 3H), 0.69-0.52 (m, 2H), 0.16-0.01 (m, 2H). |
| 68 | 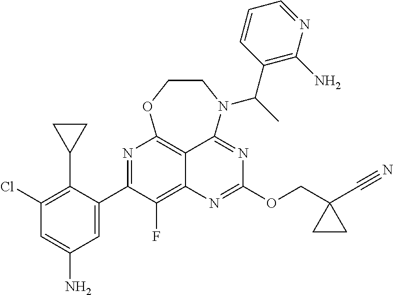 [SMILES: CC(c1cccnc1N)N1CCOc2nc(-c3cc(N)cc(Cl)c3C3CC3)c(F)c3nc(OCC4(C#N)CC4)nc1c23] | 587.3 | (400 MHz, CD₃OD/CDCl₃ = 1/1) δ 7.97-7.92 (m, 1H), 7.69-7.65 (m, 1H), 6.82-6.79 (m, 1H), 6.78-6.73 (m, 1H), 6.66-6.60 (m, 1H), 6.60-6.58 (m, 1H), 4.56-4.47 (m, 2H), 4.44-4.37 (m, 1H), 4.31-4.24 (m, 1H), 3.71- 3.62 (m, 1H), 3.55-3.46 (m, 1H), 2.00-1.79 (m, 1H), 1.67-1.60 (m, 3H), 1.42-1.37 (m, 2H), 1.28-1.26 (m, 2H), 0.68-0.56 (m, 2H), 0.11-0.07 (m, 2H). |
| 69 | 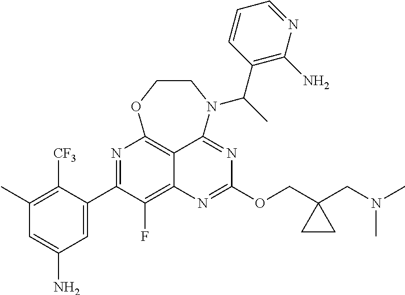 | 627.3 | (400 MHz, CD₃OD) δ 7.98-7.92 (m, 1H), 7.77-7.71 (m, 1H), 6.80-6.74 (m, 1H), 6.70-6.65 (m, 1H), 6.63- 6.54 (m, 1H), 6.44-6.37 (m, 1H), 4.47-4.25 (m, 4H), 3.81-3.67 (m, 1H), 3.61-3.47 (m, 1H), 2.86-2.64 (m, 2H), 2.54 (s, 6H), 2.43-2.37 (m, 3H), 1.72-1.59 (m, 3H), 0.87-0.79 (m, 2H), 0.68-0.60 (m, 2H). |
| 70 | 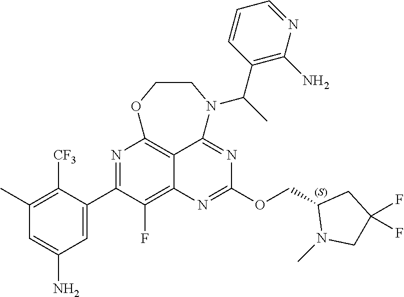 | 649.3 | (400 MHz, CD₃OD) δ 8.00-7.91 (m, 1H), 7.79-7.70 (m, 1H), 6.80-6.74 (m, 1H), 6.69-6.65 (m, 1H), 6.63- 6.56 (m, 1H), 6.41-6.35 (m, 1H), 4.62-4.24 (m, 4H), 3.80-3.67 (m, 1H), 3.60-3.46 (m, 1H), 3.43-3.34 (m, 1H), 3.14-3.03 (m, 1H), 2.83-2.68 (m, 1H), 2.61-2.48 (m, 4H), 2.43-2.37 (m, 3H), 2.32-1.99 (m, 1H), 1.69- 1.60 (m 3H). |
| 73 | 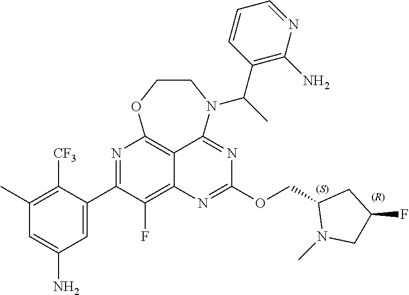 | 631.3 | (400 MHz, CD₃OD) δ 7.94 (d, J = 5.0 Hz, 1H), 7.74 (d, J = 7.5 Hz, 1H), 6.83-6.70 (m, 1H), 6.66 (s, 1H), 6.59 (t, J = 6.5 Hz, 1H), 6.38 (s, 1H), 5.22-5.14 (m, 1H), 4.63-4.20 (m, 4H), 3.76-3.64 (m, 1H), 3.65-3.41 (m, 2H), 3.20-3.10 (m, 1H), 2.73-2.53 (m, 4H), 2.40 (s, 3H), 2.36-2.21 (m, 1H), 2.13-1.89 (m, 1H), 1.64 (d, J = 6.9 Hz, 3H). |
| 82 | 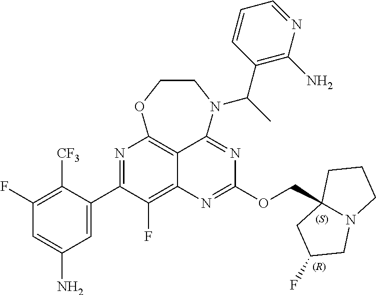 | 661.3 | (400 MHz, CD₃OD) δ 7.96-7.92 (m, 1H), 7.76-7.72 (m, 1H), 6.79-6.74 (m, 1H), 6.61-6.55 (m, 1H), 6.54- 6.49 (m, 1H), 6.41-6.34 (m, 1H), 5.39-5.21 (m, 1H), 4.48-4.40 (m, 1H), 4.35-4.24 (m, 3H), 3.78-3.68 (m, 1H), 3.57-3.47 (m, 1H), 3.28-3.16 (m, 3H), 3.04-2.96 (m, 1H), 2.40-2.10 (m, 3H), 2.03-1.84 (m, 3H), 1.66- 1.60 (m, 3H). |
| 83 | 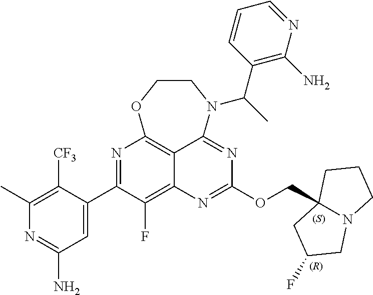 | 658.2 | (400 MHz, CD₃OD) δ 7.94 (d, J = 4.5 Hz, 1H), 7.74 (d, J = 7.3 Hz, 1H), 6.83-6.72 (m, 1H), 6.61-6.53 (m, 1H), 6.33 (s, 1H), 5.36 (d, J = 52.9 Hz, 1H), 4.49- 4.28 (m, 4H), 3.80-3.68 (m, 1H), 3.58-3.50 (m, 1H), 3.48-3.34 (m, 3H), 3.14-3.06 (m, 1H), 2.53-2.47 (m, 3H), 2.45-2.15 (m, 3H), 2.11-1.91 (m, 3H), 1.63 (d, J = 6.9 Hz, 3H). |
| 87 | 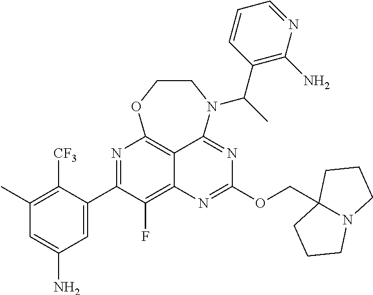 [SMILES: Cc1cc(N)cc(-c2nc3c4c(nc(OCC56CCCN5CCC6)nc4c2F)N(C(C)c2cccnc2N)CCO3)c1C(F)(F)F] | 639.4 | (400 MHz, CD₃OD) δ 7.98-7.90 (m, 1H), 7.78-7.70 (m, 1H), 6.81-6.72 (m, 1H), 6.68-6.65 (m, 1H), 6.64- 6.57 (m, 1H), 6.43-6.36 (m, 1H), 4.49-4.24 (m, 4H), 3.80-3.67 (m, 1H), 3.58-3.43 (m, 1H), 3.13-2.99 (m, 2H), 2.75-2.61 (m, 2H), 2.43-2.36 (m, 3H), 2.12-2.01 (m, 2H), 1.95-1.82 (m, 4H), 1.78-1.70 (m, 2H), 1.67- 1.60 (m, 3H). |
| 94 | 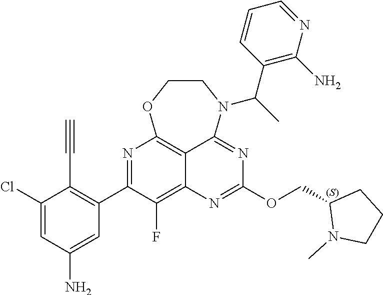 | 589.3 | (400 MHz, CD₃OD) δ 7.97-7.90 (m, 1H), 7.77-7.70 (m, 1H), 6.82-6.79 (m, 1H), 6.79-6.73 (m, 1H), 6.64- 6.55 (m, 2H), 4.59-4.38 (m, 3H), 4.36-4.24 (m, 1H), 3.77-3.66 (m, 1H), 3.55-3.46 (m, 1H), 3.41 (s, 1H), 3.13-3.02 (m, 1H), 2.89-2.73 (m, 1H), 2.52 (s, 3H), 2.41-2.31 (m, 1H), 2.17-2.06 (m, 1H), 1.88-1.72 (m, 3H), 1.68-1.56 (m, 3H). |
| 97 | 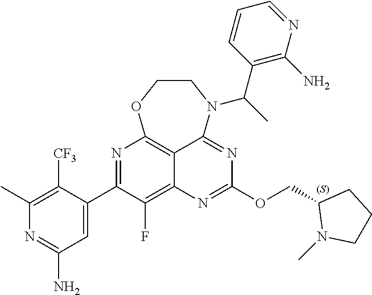 | 614.2 | (400 MHz, CD₃OD) δ 7.94 (d, J = 5.2 Hz, 1H), 7.75 (d, J = 7.4 Hz, 1H), 6.81-6.74 (m, 1H), 6.60 (d, J = 6.7 Hz, 1H), 6.32 (s, 1H), 4.58-4.41 (m, 3H), 4.36- 4.26 (m, 1H), 3.79-3.68 (m, 1H), 3.55-3.47 (m, 1H), 3.13-3.05 (m, 1H), 2.85-2.78 (m, 1H), 2.53-2.51 (m, 6H), 2.40-2.32 (m, 1H), 2.16-2.05 (m, 1H), 1.88-1.76 (m, 3H), 1.64 (d, J = 6.9 Hz, 3H). |
| 104 | 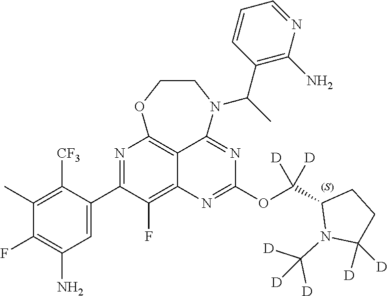 | 638.3 | (400 MHz, CD₃OD) δ 7.96-7.92 (m, 1H), 7.76-7.72 (m, 1H), 6.79-6.74 (m, 1H), 6.64-6.54 (m, 2H), 4.49- 4.24 (m, 2H), 3.78-3.65 (m, 1H), 3.56-3.46 (m, 1H), 2.83-2.76 (m, 1H), 2.39-2.34 (m, 3H), 2.20-1.72 (m, 4H), 1.66-1.61 (m, 3H). |
| 105 | 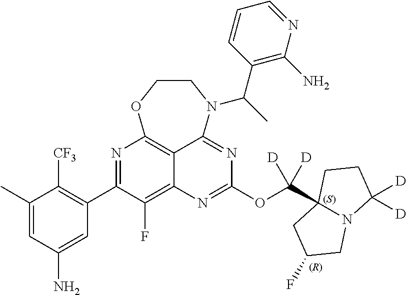 | 661.3 | (400 MHz, CD₃OD) δ 7.94 (d, J = 4.4 Hz, 1H), 7.74 (d, J = 7.3 Hz, 1H), 6.78-6.74 (m, 1H), 6.66 (s, 1H), 6.58 (q, J = 6.6 Hz, 1H), 6.39 (s, 1H), 5.40-5.21 (m, 1H), 4.50-4.20 (m, 2H), 3.76-3.70 (m, 1H), 3.54-3.48 (m, 1H), 3.30-3.18 (m, 2H), 2.39 (s, 3H), 2.37-2.08 (m, 3H), 2.04-1.80 (m, 3H), 1.63 (d, J = 6.8 Hz, 3H). |
| 106 | 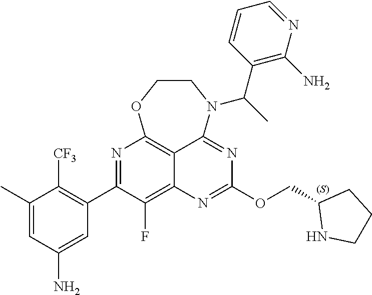 | 599.3 | (400 MHz, CD₃OD) δ 7.94-7.90 (m, 1H), 7.72 (d, J = 7.0 Hz, 1H), 6.77-6.73 (m, 1H), 6.68-6.62 (m, 2H), 6.37 (s, 1H), 4.43-4.054 (m, 3H), 3.90-3.60 (m, 5H), 3.51-3.35 (m, 1H), 2.41-2.37 (m, 3H), 2.22-1.87 (m, 4H), 1.64-1.59 (m, 3H). ISCO (MeOH/H₂O, 0.1% HCOOH), peak 1 |
| | + | | |
| 107 | 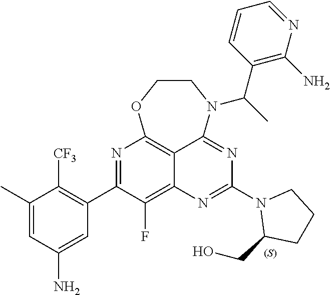 | 599.3 | (400 MHz, CD₃OD) δ 7.94-7.90 (m, 1H), 7.71 (d, J = 7.5 Hz, 1H), 6.85-6.69 (m, 1H), 6.68-6.62 (m, 2H), 6.38 (s, 1H), 4.49-4.17 (m, 3H), 4.00-3.55 (m, 5H), 3.46-3.40 (m, 1H), 2.41-2.37 (m, 3H), 2.22-1.82 (m, 4H), 1.66-1.54 (m, 3H). ISCO (MeOH/H₂O, 0.1% HCOOH), peak 2 |
| 108 | 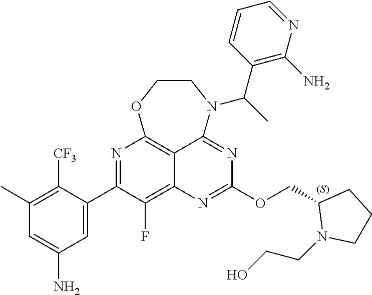 | 643.3 | (400 MHz, CD₃OD) δ 7.97-7.91 (m, 1H), 7.79-7.70 (m, 1H), 6.82-6.72 (m, 1H), 6.68-6.58 (m, 2H), 6.42- 6.35 (m, 1H), 4.59-4.08 (m, 4H), 3.76-3.62 (m, 3H), 3.57-3.45 (m, 2H), 3.23-3.13 (m, 2H), 3.07-2.93 (m, 1H), 2.60-2.50 (m, 1H), 2.47-2.36 (m, 4H), 2.27-1.99 (m, 1H), 1.86-1.74 (m, 2H), 1.68-1.62 (m, 3H). |
| 111 | 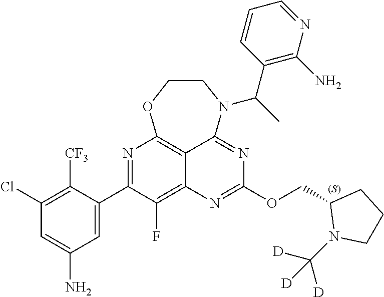 | 636.4 | (400 MHz, CD₃OD) δ 7.94 (d, J = 5.0 Hz, 1H), 7.74 (d, J = 7.6 Hz, 1H), 6.86 (d, J = 2.1 Hz, 1H), 6.79- 6.74 (m, 1H), 6.60 (d, J = 4.9 Hz, 1H), 6.47 (d, J = 1.7 Hz, 1H), 4.59-4.48 (m, 2H), 4.43 (d, J = 6.8 Hz, 1H), 4.38-4.26 (m, 1H), 3.71 (s, 1H), 3.51 (d, J = 15.3 Hz, 1H), 3.14-3.06 (m, 1H), 2.91-2.81 (m, 1H), 2.43-2.34 (m, 1H), 2.17-2.07 (m, 1H), 1.88-1.73 (m, 3H), 1.64 (d, J = 6.9 Hz, 3H). |
| 112 | 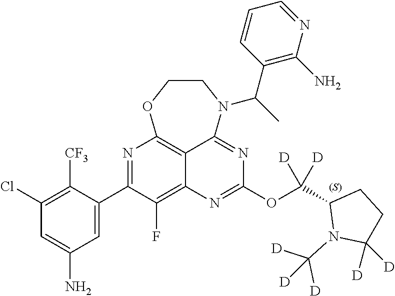 | 640.4 | (400 MHz, CD₃OD) δ 7.94 (d, J = 4.9 Hz, 1H), 7.75 (d, J = 7.6 Hz, 1H), 6.86 (d, J = 2.2 Hz, 1H), 6.81- 6.74 (m, 1H), 6.60 (d, J = 7.0 Hz, 1H), 6.46 (s, 1H), 4.43 (s, 1H), 4.37-4.25 (m, 1H), 3.71 (s, 1H), 3.53 (s, 1H), 2.83-2.74 (m, 1H), 2.18-2.07 (m, 1H), 1.87-1.74 (m, 3H), 1.64 (d, J = 6.9 Hz, 3H). |
| 113 | 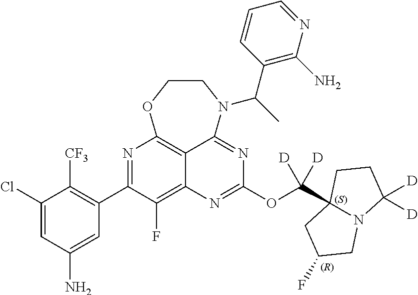 | 681.4 | (400 MHz, CD₃OD) δ 7.94 (d, J = 4.6 Hz, 1H), 7.73 (d, J = 7.4 Hz, 1H), 6.86 (d, J = 2.1 Hz, 1H), 6.79- 6.72 (m, 1H), 6.58 (d, J = 6.7 Hz, 1H), 6.46 (d, J = 1.9 Hz, 1H), 5.41-5.20 (m, 1H), 4.49-4.38 (m, 1H), 4.36-4.26 (m, 1H), 3.77-3.67 (m, 1H), 3.58-3.45 (m, 1H), 3.29-3.19 (m, 1H), 3.18-3.14 (m, 1H), 2.42-2.17 (m, 2H), 2.16-2.09 (m, 1H), 2.01-1.92 (m, 2H), 1.91- 1.81 (m, 1H), 1.63 (d, J = 6.9 Hz, 3H). |
| 114 | 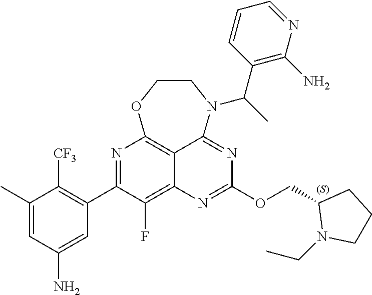 | 627.3 | (400 MHz, CD₃OD) δ 7.98-7.91 (m, 1H), 7.80-7.71 (m, 1H), 6.82-6.73 (m, 1H), 6.66 (s, 1H), 6.64-6.57 (m, 1H), 6.38 (s, 1H), 4.62-4.50 (m, 1H), 4.49-4.37 (m, 2H), 4.37-4.21 (m, 1H), 3.78-3.65 (m, 1H), 3.56- 3.44 (m, 1H), 3.26-3.06 (m, 2H), 3.06-2.91 (m, 1H), 2.51-2.42 (m, 1H), 2.42-2.37 (m, 3H), 2.37-2.27 (m, 1H), 2.15-2.02 (m, 1H), 1.91-1.75 (m, 3H), 1.68-1.59 (m, 3H), 1.19-1.11 (m, 3H). |
| 119 | 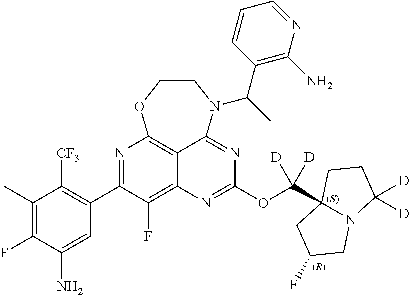 | 679.3 | (400 MHz, CD₃OD) δ 7.97-7.90 (m, 1H), 7.77-7.69 (m, 1H), 6.80-6.72 (m, 1H), 6.63-6.51 (m, 2H), 5.41- 5.23 (m, 1H), 4.50-4.24 (m, 2H), 3.75-3.67 (m, 1H), 3.57-3.46 (m, 1H), 3.29-3.21 (m, 2H), 2.43-2.11 (m, 6H), 2.04-1.85 (m, 3H), 1.67-1.58 (m, 3H). |
| 120 | 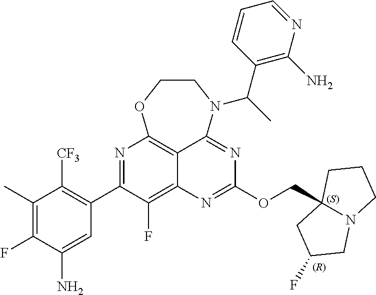 | 675.3 | (400 MHz, CD₃OD) δ 7.96-7.92 (m, 1H), 7.77-7.71 (m, 1H), 6.80-6.73 (m, 1H), 6.63-6.53 (m, 2H), 5.41- 5.23 (m, 1H), 4.50-4.24 (m, 4H), 3.79-3.68 (m, 1H), 3.58-3.47 (m, 1H), 3.39-3.30 (m, 1H), 3.28-3.18 (m, 2H), 3.09-2.98 (m, 1H), 2.43-2.10 (m, 6H), 2.05-1.87 (m, 3H), 1.66-1.60 (m, 3H). |
| 121 | 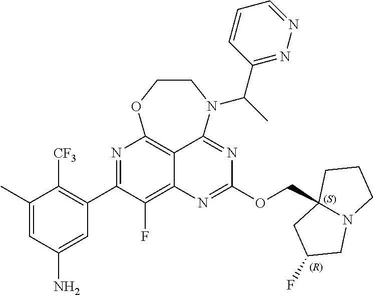 | 643.3 | (400 MHz, CD₃OD) δ 9.15-9.08 (m, 1H), 7.87-7.78 (m, 1H), 7.75-7.66 (m, 1H), 6.71-6.57 (m, 2H), 6.44- 6.36 (m, 1H), 5.36-5.14 (m, 1H), 4.68-4.52 (m, 2H), 4.13-3.86 (m, 4H), 3.27-3.11 (m, 3H), 3.00-2.92 (m, 1H), 2.43-2.35 (m, 3H), 2.30-1.99 (m, 3H), 1.98-1.75 (m, 6H). |
| 122 | 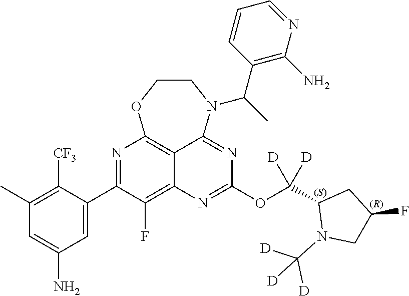 | 636.4 | (400 MHz, CD₃OD) δ 7.94 (d, J = 5.1 Hz, 1H), 7.74 (d, J = 7.5 Hz, 1H), 6.83-6.73 (m, 1H), 6.66 (s, 1H), 6.64-6.56 (m, 1H), 6.38 (s, 1H), 5.28-5.07 (m, 1H), 4.48-4.20 (m, 2H), 3.78-3.66 (m, 1H), 3.56-3.43 (m, 2H), 3.18-3.10 (m, 1H), 2.72-2.59 (m, 1H), 2.40 (d, J = 2.1 Hz, 3H), 2.35-2.23 (m, 1H), 2.11-1.93 (m, 1H), 1.64 (d, J = 6.9 Hz, 3H). |
| 123 | 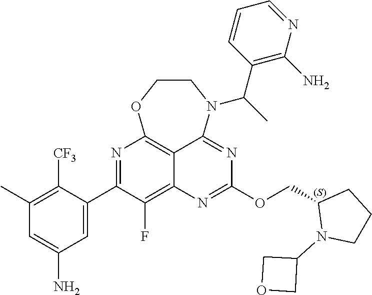 | 655.4 | (400 MHz, CD₃OD) δ 7.95 (d, J = 4.9 Hz, 1H), 7.75 (d, J = 7.3 Hz, 1H), 6.81-6.74 (m, 1H), 6.66 (s, 1H), 6.59 (q, J = 7.0 Hz, 1H), 6.38 (s, 1H), 4.87-4.77 (m, 1H), 4.75-4.66 (m, 2H), 4.46-4.31 (m, 3H), 4.11- 3.98 (m, 1H), 3.79-3.67 (m, 1H), 3.56-3.46 (m, 1H), 3.41-3.32 (m, 1H), 3.10-3.02 (m, 1H), 3.02-2.72 (m, 1H), 2.60-2.43 (m, 1H), 2.40 (s, 3H), 2.36-2.21 (m, 1H), 2.14-1.96 (m, 1H), 1.89-1.74 (m, 3H), 1.64 (d, J = 6.9 Hz, 3H). |
| 126 | 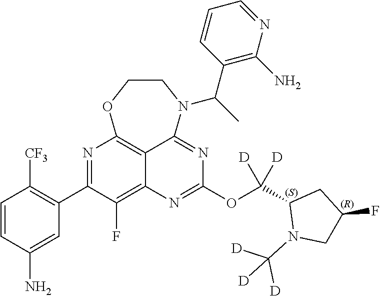 | 622.3 | (400 MHz, CD₃OD) δ 7.97-7.90 (m, 1H), 7.76-7.69 (m, 1H), 7.47-7.40 (m, 1H), 6.82-6.72 (m, 2H), 6.65- 6.55 (m, 2H), 5.28-5.06 (m, 1H), 4.46-4.25 (m, 2H), 3.76-3.65 (m, 1H), 3.57-3.43 (m, 2H), 3.20-3.12 (m, 1H), 2.75-2.58 (m, 1H), 2.36-2.23 (m, 1H), 2.11-1.93 (m, 1H), 1.68-1.57 (m, 3H). |
| 129 | 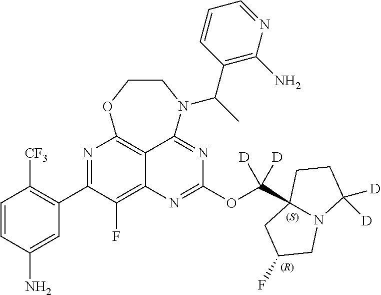 | 647.3 | (400 MHz, CD₃OD) δ 7.94 (d, J = 4.6 Hz, 1H), 7.71 (d, J = 8.8 Hz, 1H), 7.44 (d, J = 8.7 Hz, 1H), 6.84- 6.70 (m, 2H), 6.68-6.51 (m, 2H), 5.40-5.20 (m, 1H), 4.49-4.21 (m, 2H), 3.78-3.64 (m, 1H), 3.56-3.46 (m, 1H), 3.29-3.11 (m, 2H), 2.42-2.08 (m, 3H), 2.02-1.78 (m, 3H), 1.62 (d, J = 6.8 Hz, 3H). |
| 134 | 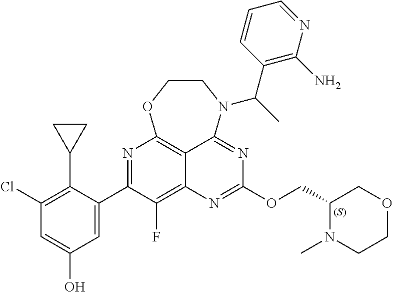 | 622.3 | (400 MHz, CD₃OD) δ 7.97-7.57 (m, 2H), 6.89 (d, J = 2.6 Hz, 1H), 6.80-6.74 (m, 1H), 6.72-6.68 (m, 1H), 6.66-6.56 (m, 1H), 4.68-4.26 (m, 4H), 4.02-3.91 (m, 1H), 3.81-3.60 (m, 3H), 3.57-3.48 (m, 2H), 2.80-2.71 (m, 1H), 2.68-2.58 (m, 1H), 2.48-2.35 (m, 4H), 1.89- 1.77 (m, 1H), 1.64 (d, J = 6.9 Hz, 3H), 0.70-0.56 (m, 2H), 0.18-0.05 (m, 2H). |
| 135 | 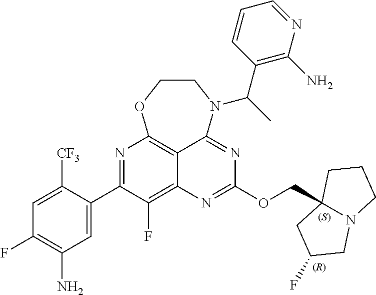 | 661.3 | (400 MHz, CD₃OD) δ 7.94 (d, J = 4.1 Hz, 1H), 7.73 (d, J = 7.5 Hz, 1H), 7.34 (d, J = 11.8 Hz, 1H), 6.82 (d, J = 8.5 Hz, 1H), 6.78-6.72 (m, 1H), 6.61-6.54 (m, 1H), 5.44-5.13 (m, 1H), 4.48-4.38 (m, 1H), 4.36-4.23 (m, 3H), 3.80-3.68 (m, 1H), 3.58-3.47 (m, 1H), 3.28- 3.12 (m, 3H), 3.05-2.95 (m, 1H), 2.38-2.16 (m, 2H), 2.14-2.07 (m, 1H), 2.01-1.93 (m, 2H), 1.92-1.79 (m, 1H), 1.63 (d, J = 6.9 Hz, 3H). |
| 136 | 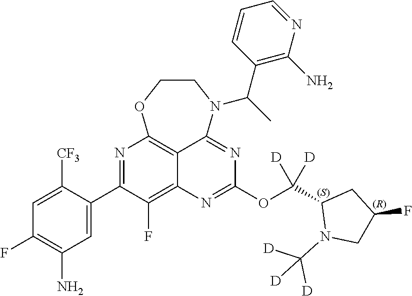 | 640.3 | (400 MHz, CD₃OD) δ 7.94 (d, J = 5.0 Hz, 1H), 7.73 (d, J = 7.5 Hz, 1H), 7.35 (d, J = 11.8 Hz, 1H), 6.82 (d, J = 8.5 Hz, 1H), 6.79-6.73 (m, 1H), 6.63-6.55 (m, 1H), 5.26-5.08 (m, 1H), 4.48-4.37 (m, 1H), 4.35-4.25 (m, 1H), 3.76-3.67 (m, 1H), 3.55-3.44 (m, 2H), 3.17- 3.09 (m, 1H), 2.73-2.58 (m, 1H), 2.35-2.21 (m, 1H), 2.12-1.95 (m, 1H), 1.63 (d, J = 6.9 Hz, 3H). |
| 139 | 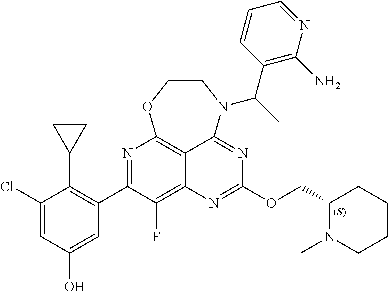 | 620.6 | (400 MHz, CD₃OD) δ 7.98-7.91 (m, 1H), 7.78-7.72 (m, 1H), 6.94-6.89 (m, 1H), 6.80-6.75 (m, 1H), 6.75- 6.70 (m, 1H), 6.66-6.57 (m, 1H), 4.63-4.53 (m, 2H), 4.49-4.40 (m, 1H), 4.34-4.27 (m, 1H), 3.80-3.68 (m, 1H), 3.60-3.56 (m, 1H), 3.56-3.48 (m, 1H), 2.94-2.88 (m, 1H), 2.51-2.44 (m, 1H), 2.44-2.41 (m, 3H), 2.28- 2.16 (m, 1H), 1.92-1.77 (m, 3H), 1.66-1.63 (m, 3H), 1.62-1.46 (m, 2H), 1.42-1.34 (m, 1H), 0.75-0.55 (m, 2H), 0.18-0.05 (m, 2H). |
| 140 | 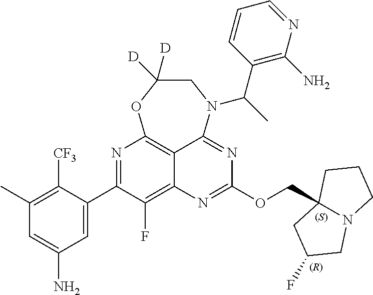 | 659.3 | (400 MHz, CD₃OD) δ 7.94 (d, J = 4.0 Hz, 1H), 7.73 (d, J = 7.3 Hz, 1H), 6.80-6.73 (m, 1H), 6.66 (s, 1H), 6.63-6.54 (m, 1H), 6.39 (d, J = 1.8 Hz, 1H), 5.43- 5.17 (m, 1H), 4.38-4.21 (m, 2H), 3.71 (d, J = 16.0 Hz, 1H), 3.50 (d, J = 15.9 Hz, 1H), 3.28-3.11 (m, 3H), 3.07-2.95 (m, 1H), 2.44-2.36 (m, 3H), 2.36-2.08 (m, 3H), 2.02-1.93 (m, 2H), 1.93-1.82 (m, 1H), 1.63 (d, J = 6.9 Hz, 3H). |
| 141 | 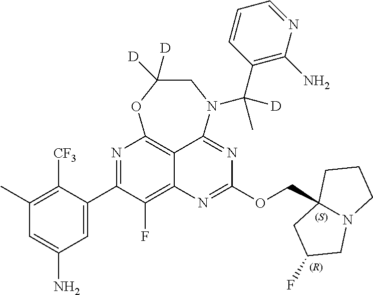 | 600.3 | (400 MHz, CD₃OD) δ 7.97-7.92 (m, 1H), 7.77-7.71 (m, 1H), 6.80-6.72 (m, 1H), 6.70-6.64 (m, 1H), 6.43- 6.35 (m, 1H), 5.41-5.19 (m, 1H), 4.37-4.23 (m, 2H), 3.75-3.66 (m, 1H), 3.54-3.45 (m, 1H), 3.28-3.14 (m, 3H), 3.03-2.94 (m, 1H), 2.44-2.38 (m, 3H), 2.37-2.17 (m, 2H), 2.16-2.07 (m, 1H), 2.02-1.94 (m, 2H), 1.93- 1.84 (m, 1H), 1.65-1.60 (m, 3H). |
| 146 | 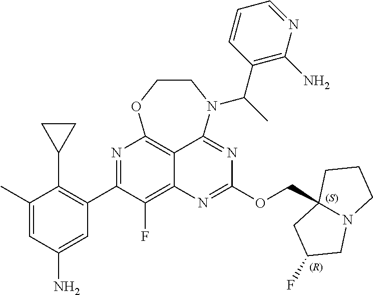 | 629.4 | (400 MHz, CD₃OD) δ 7.95 (d, J = 4.6 Hz, 1H), 7.74 (d, J = 7.0 Hz, 1H), 6.80-6.74 (m, 1H), 6.68-6.64 (m, 1H), 6.64-6.56 (m, 1H), 6.56-6.52 (m, 1H), 5.41-5.21 (m, 1H), 4.49-4.39 (m, 1H), 4.39-4.23 (m, 3H), 3.78- 3.68 (m, 1H), 3.57-3.48 (m, 1H), 3.25-3.18 (m, 2H), 3.06-2.97 (m, 1H), 2.39 (s, 3H), 2.30-2.22 (m, 1H), 2.18-2.11 (m, 1H), 2.04-1.95 (m, 2H), 1.94-1.86 (m, 3H), 1.83-1.75 (m, 1H), 1.64 (d, J = 6.9 Hz, 3H), 0.67-0.42 (m, 2H), 0.07-0.09 (m, 2H). |
| 148 | 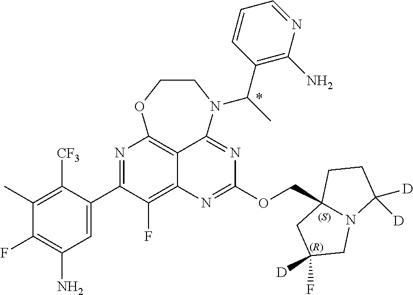 | 678.3 | (400 MHz, CD₃OD) δ 7.93 (d, J = 3.9 Hz, 1H), 7.78- 7.60 (m, 1H), 6.78-6.72 (m, 1H), 6.65-6.51 (m, 2H), 4.50-4.21 (m, 4H), 3.75-3.69 (m, 1H), 3.52-3.48 (m, 1H), 3.28-3.10 (m, 2H), 2.40-2.05 (m, 6H), 2.03-1.80 (m, 3H), 1.62 (d, J = 6.9 Hz, 3H). |
| 149 | 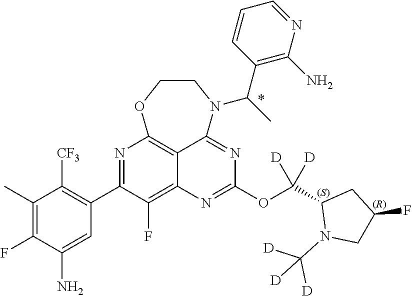 | 654.3 | (400 MHz, CD₃OD) δ 7.97-7.90 (m, 1H), 7.76-7.70 (m, 1H), 6.79-6.72 (m, 1H), 6.64-6.51 (m, 2H), 5.28- 5.07 (m, 1H), 4.47-4.24 (m, 2H), 3.78-3.63 (m, 1H), 3.58-3.44 (m, 2H), 3.24-3.14 (m, 1H), 2.78-2.60 (m, 1H), 2.39-2.24 (m, 4H), 2.11-1.94 (m, 1H), 1.68-1.58 (m, 3H). |
| 150 | 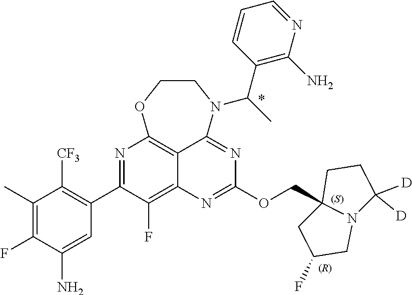 | 677.3 | (400 MHz, CD₃OD) δ 7.97-7.90 (m, 1H), 7.75-7.70 (m, 1H), 6.78-6.73 (m, 1H), 6.63-6.52 (m, 2H), 5.39- 5.21 (m, 1H), 4.47-4.25 (m, 4H), 3.78-3.66 (m, 1H), 3.56-3.46 (m, 1H), 3.32-3.18 (m, 2H), 2.40-2.11 (m, 6H), 2.04-1.85 (m, 3H), 1.66-1.58 (m, 3H). |
| 157 | 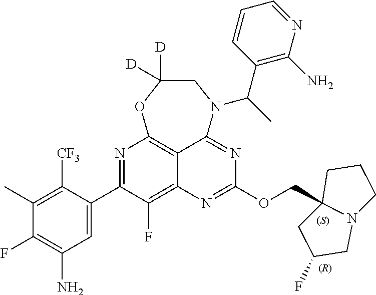 | 677.3 | (400 MHz, CD₃OD) δ 7.98-7.91 (m, 1H), 7.76-7.70 (m, 1H), 6.80-6.73 (m, 1H), 6.62-6.54 (m, 2H), 5.42- 5.19 (m, 1H), 4.37-4.23 (m, 2H), 3.77-3.67 (m, 1H), 3.55-3.46 (m, 1H), 3.27-3.14 (m, 3H), 3.04-2.96 (m, 1H), 2.38-2.35 (m, 3H), 2.23-2.06 (m, 3H), 2.02-1.86 (m, 3H), 1.63 (d, J = 6.9 Hz, 3H). |
| 158 | 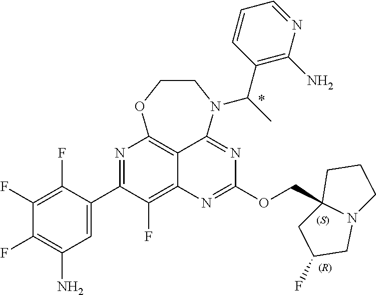 | 629.3 | (400 MHz, CD₃OD) δ 7.96-7.89 (m, 1H), 7.73 (d, J = 7.5 Hz, 1H), 6.81-6.71 (m, 2H), 6.62-6.51 (m, 1H), 5.42-5.18 (m, 1H), 4.51-4.40 (m, 1H), 4.37-4.25 (m, 3H), 3.78-3.67 (m, 1H), 3.5-3.45 (m, 1H), 3.27-3.13 (m, 3H), 3.04-2.94 (m, 1H), 2.36-2.09 (m, 3H), 2.03- 1.86 (m, 3H), 1.62 (d, J = 6.9 Hz, 3H). |
| 159 | 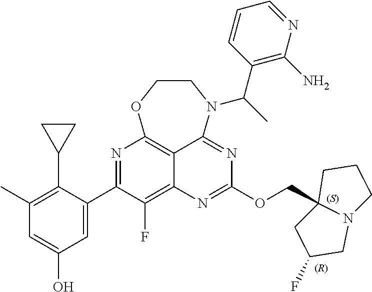 | 630.2 | (400 MHz, CD₃OD) δ 7.94 (d, J = 5.1 Hz, 1H), 7.74 (d, J = 7.6 Hz, 1H), 6.80-6.74 (m, 1H), 6.73-6.68 (m, 1H), 6.64-6.54 (m, 2H), 5.41-5.20 (m, 1H), 4.49-4.40 (m, 1H), 4.38-4.24 (m, 3H), 3.80-3.69 (m, 1H), 3.58- 3.48 (m, 1H), 3.26-3.18 (m, 2H), 3.06-2.97 (m, 1H), 2.45-2.38 (m, 3H), 2.38-2.17 (m, 2H), 2.18-2.11 (m, 1H), 2.04-1.95 (m, 2H), 1.94-1.74 (m, 3H), 1.64 (d, J = 6.8 Hz, 3H), 0.70-0.49 (m, 2H), 0.08-0.03 (m, 2H). |
| 163 | 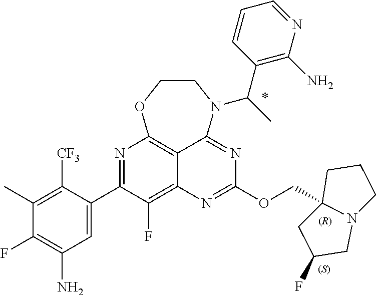 | 675.3 | (400 MHz, CD₃OD) δ 7.97-7.92 (m, 1H), 7.79-7.70 (m, 1H), 6.82-6.73 (m, 1H), 6.63-6.52 (m, 2H), 5.46- 5.17 (m, 1H), 4.48-4.25 (m, 4H), 3.83-3.64 (m, 2H), 3.57-3.44 (m, 1H), 3.25-3.17 (m, 2H), 3.07-2.97 (m, 1H), 2.42-2.35 (m, 3H), 2.30-2.09 (m, 3H), 2.05-1.86 (m, 3H), 1.71-1.59 (m, 3H). |
| 164 | 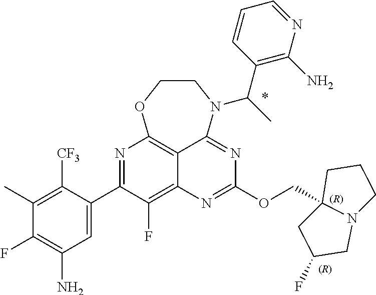 | 675.3 | (400 MHz, CD₃OD) δ 7.99-7.90 (m, 1H), 7.81-7.69 (m, 1H), 6.83-6.72 (m, 1H), 6.68-6.52 (m, 2H), 5.42- 5.20 (m, 1H), 4.51-4.38 (m, 3H), 4.38-4.22 (m, 1H), 3.77-3.65 (m, 1H), 3.55-3.36 (m, 2H), 3.14-3.03 (m, 1H), 2.99-2.81 (m, 1H), 2.77-2.68 (m, 1H), 2.57-2.43 (m, 1H), 2.37 (s, 3H), 2.19-2.09 (m, 1H), 2.07-1.88 (m, 3H), 1.88-1.79 (m, 1H), 1.71-1.54 (m, 3H). |
| 165 | 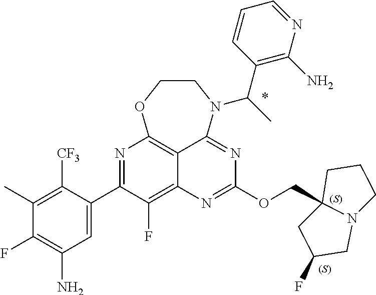 | 675.3 | (400 MHz, CD₃OD) δ 8.01-7.88 (m, 1H), 7.82-7.68 (m, 1H), 6.85-6.71 (m, 1H), 6.69-6.52 (m, 2H), 5.42- 5.19 (m, 1H), 4.56-4.19 (m, 4H), 3.82-3.62 (m, 1H), 3.53-3.35 (m, 2H), 3.12-3.02 (m, 1H), 2.97-2.80 (m, 1H), 2.79-2.68 (m, 1H), 2.57-2.44 (m, 1H), 2.37 (s, 3H), 2.19-2.10 (m, 1H), 2.04-1.89 (m, 3H), 1.87-1.78 (m, 1H), 1.69-1.57 (m, 3H). |
| 168 | 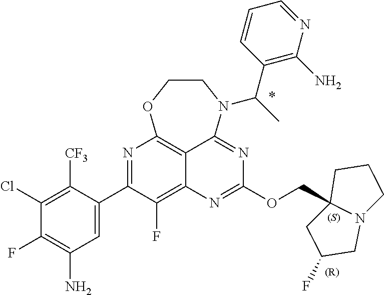 | 695.2 | (400 MHz, CD₃OD) δ 7.95 (d, J = 4.8 Hz, 1H), 7.74 (d, J = 7.2 Hz, 1H), 6.82-6.74 (m, 1H), 6.71-6.64 (m, 1H), 6.63-6.54 (m, 1H), 5.41-5.21 (m, 1H), 4.49-4.40 (m, 1H), 4.39-4.27 (m, 3H), 3.81-3.67 (m, 1H), 3.58- 3.48 (m, 1H), 3.28-3.16 (m, 3H), 3.06-2.97 (m, 1H), 2.39-2.12 (m, 3H), 2.05-1.87 (m, 3H), 1.64 (d, J = 6.7 Hz, 3H). |
| 171 | 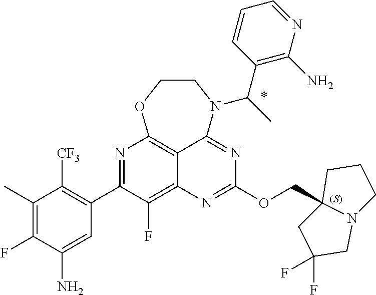 | 693.2 | (400 MHz, CD₃OD) δ 8.01-7.84 (m, 1H), 7.72 (d, J = 6.9 Hz, 1H), 6.78-6.72 (m, 1H), 6.67-6.42 (m, 2H), 4.50-4.15 (m, 4H), 3.80-3.62 (m, 1H), 3.56-3.35 (m, 2H), 3.23-3.01 (m, 2H), 2.93-2.75 (m, 1H), 2.65-2.55 (m, 1H), 2.41-2.07 (m, 5H), 2.04-1.83 (m, 3H), 1.63 (d, J = 6.9 Hz, 3H). |
| 173 | 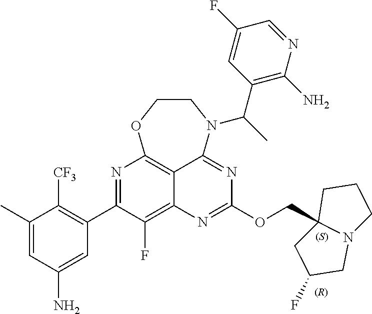 | 675.3 | (400 MHz, CD₃OD) δ 7.88 (s, 1H), 7.62 (d, J = 9.1 Hz, 1H), 6.67 (s, 1H), 6.57 (d, J = 5.5 Hz, 1H), 6.38 (s, 1H), 5.43-5.25 (m, 1H), 4.47-4.25 (m, 4H), 3.81- 3.72 (m, 1H), 3.59-3.51(m, 1H), 3.48-3.31 (m, 3H), 3.12-3.04 (m, 1H), 2.43-2.15 (m, 6H), 2.07-1.89 (m, 3H), 1.63 (d, J = 6.9 Hz, 3H). |
| 174 | 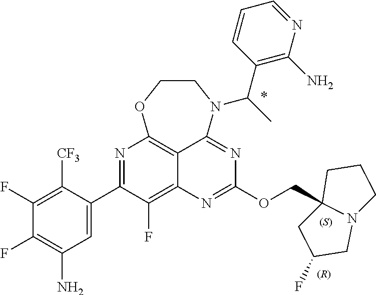 | 679.3 | (400 MHz, CD₃OD) δ 7.99-7.92 (m, 1H), 7.73 (d, J = 7.5 Hz, 1H), 6.82-6.72 (m, 1H), 6.64-6.54 (m,2H), 5.37-5.17 (m, 1H), 4.50-4.39 (m, 1H), 4.36-4.26 (m, 3H), 3.78-3.68 (m, 1H), 3.58-3.47 (m, 1H), 3.25-3.10 (m, 3H), 3.03-2.91 (m, 1H), 2.38-2.12 (m, 3H), 2.01- 1.89 (m, 3H), 1.63 (d, J = 6.9 Hz, 3H). |
| 177 | 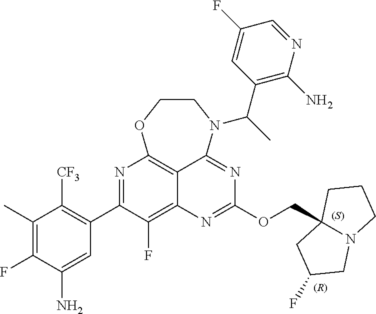 | 693.2 | (400 MHz, CD₃OD) δ 7.88 (d, J = 2.7 Hz, 1H), 7.62 (dd, J = 9.2, 2.6 Hz, 1H), 6.57 (d, J = 8.2 Hz, 2H), 5.41-5.20 (m, 1H), 4.50-4.24 (m, 4H), 3.82-3.69 (m, 1H), 3.58-3.48 (m, 1H), 3.26-3.13 (m, 3H), 3.05-2.96 (m, 1H), 2.37 (s, 3H), 2.34-2.08 (m, 3H), 2.03-1.85 (m, 3H), 1.63 (d, J = 6.9 Hz, 3H). |
| 180 | 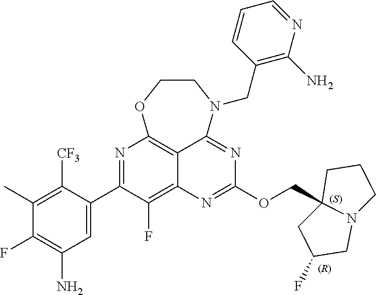 | 661.3 | (400 MHz, CDCl₃&CD₃OD) δ 7.95-7.86 (m, 1H), 7.47-7.39 (m, 1H), 6.68-6.60 (m, 1H), 6.59-6.51 (m, 1H), 5.33-5.16 (m, 1H), 5.08-4.89 (m, 2H), 4.57-4.44 (m, 2H), 4.22-4.10 (m, 2H), 3.90 (s, 2H), 3.23-3.15 (m, 2H), 3.15-3.06 (m, 1H), 3.04-2.87 (m, 1H), 2.35 (s, 3H), 2.27-2.16 (m, 1H), 2.14-2.12 (m, 1H), 2.08- 2.00 (m, 1H), 1.98-1.88 (m, 2H), 1.87-1.73 (m, 1H). |
| 181 | 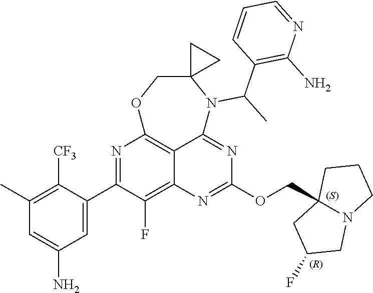 | 683.3 | (400 MHz, CD₃OD) δ 8.06-7.89 (m, 1H), 7.80-7.54 (m, 1H), 6.80-6.71 (m, 1H), 6.67 (s, 1H), 6.43 (s, 1H), 6.20-6.09 (m, 1H), 5.40-5.16 (m, 1H), 4.73-4.43 (m, 1H), 4.40-4.04 (m, 3H), 3.28-3.13 (m, 3H), 3.02- 2.95 (m, 1H), 2.47-2.37 (m, 3H), 2.34-2.05 (m, 3H), 2.00-1.91 (m, 2H), 1.91-1.83 (m, 1H), 1.83-1.76 (m, 3H), 1.30-0.85 (m, 4H). |
| 185 | 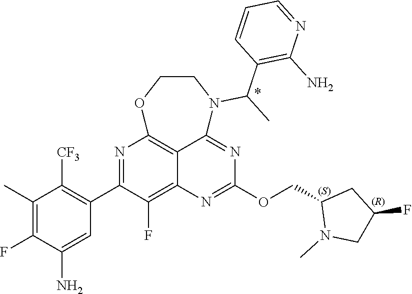 | 649.3 | (400 MHz, CD₃OD) δ 7.97-7.91 (m, 1H), 7.76-7.71 (m, 1H), 6.79-6.74 (m, 1H), 6.65-6.53 (m, 2H), 5.27- 5.07 (m, 1H), 4.62-4.48 (m, 2H), 4.48-4.23 (m, 2H), 3.76-3.65 (m, 1H), 3.56-3.44 (m, 2H), 3.20-3.11 (m, 1H), 2.71-2.59 (m, 1H), 2.55 (s, 3H), 2.39-2.35 (m, 3H), 2.35-2.22 (m, 1H), 2.10-1.93 (m, 1H), 1.68-1.59 (m, 3H). |
| 187 | 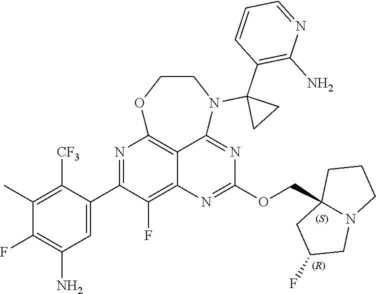 | 687.3 | (400 MHz, CD₃OD) δ 7.91-7.82 (m, 2H), 6.70-6.61 (m, 1H), 6.55 (d, J = 8.6 Hz, 1H), 5.44-5.23 (m, 1H), 4.58-4.46 (m, 2H), 4.38-4.25 (m, 2H), 4.24-4.11 (m, 2H), 3.29-3.16 (m, 3H), 3.08-2.99 (m, 1H), 2.40-2.34 (m, 3H), 2.32-2.10 (m, 3H), 2.05-1.90 (m, 3H), 1.69- 1.56 (m, 2H), 1.51-1.35 (m, J = 19.8 Hz, 2H). |
| 195 | 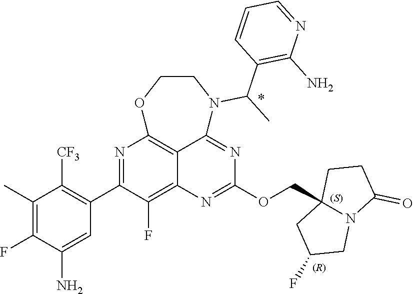 | 689.3 | (400 MHz, CD₃OD) δ 7.98-7.90 (m, 1H), 7.75-7.68 (m, 1H), 6.79-6.73 (m, 1H), 6.64-6.48 (m, 2H), 5.44- 5.28 (m, 1H), 4.61-4.54 (m, 1H), 4.52-4.39 (m, 2H), 4.38-3.24 (m, 1H), 4.08-3.96 (m, 1H), 3.80-3.68 (m, 1H), 3.59-3.50 (m, 1H), 3.38-3.20 (m, 1H), 3.10-2.96 (m, 1H), 2.51-2.34 (m, 6H), 2.22-2.10 (m, 2H), 1.67- 1.58 (m, 3H). |
| 196 | 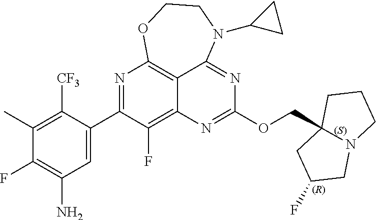 | 595.2 | (400 MHz, CD₃OD) δ 6.58 (d, J = 8.6 Hz, 1H), 5.43- 5.15 (m, 1H), 4.66-4.51 (m, 2H), 4.44-4.24 (m, 2H), 4.12-3.97 (m, 2H), 3.28-3.12 (m, 4H), 3.08-2.95 (m, 1H), 2.41-2.31 (m,3H), 2.33-2.10 (m, 3H), 2.08-1.81 (m, 3H), 1.15-1.01 (m, 2H), 0.92-0.75 (m, 2H). |
| 197 | 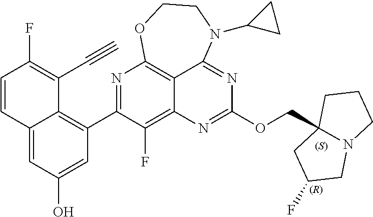 | 588.3 | (400 MHz, CD₃OD&CDCl₃) δ 8.28-8.22 (m, 1H), 7.74-7.68 (m, 1H), 7.26-7.18 (m, 2H), 7.17-7.13 (m, 1H), 5.39-5.16 (m, 1H), 4.55-4.54 (m, 1H), 4.35-4.30 (m, 1H), 4.25-4.21 (m, 1H), 4.02-3.95 (m, 2H), 3.25- 3.19 (m, 2H), 3.17-3.10 (m, 2H), 3.02-2.93 (m, 1H), 2.33-2.23 (m, 1H), 2.22-2.17 (m, 1H), 2.15-2.08 (m, 1H), 2.01-1.92 (m, 2H), 1.92-1.74 (m, 2H), 1.11-1.03 (m, 2H), 0.82-0.74 (m, 2H). |
| 198 | 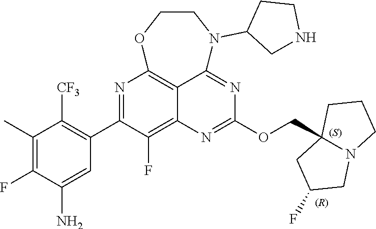 | 624.2 | (400 MHz, CD₃OD) δ 6.61-6.56 (m, 1H), 5.66-5.49 (m, 1H), 5.40-5.18 (m, 1H), 4.64-4.52 (m, 2H), 4.32- 4.20 (m, 2H), 4.01-3.89 (m, 2H), 3.29-3.15 (m, 5H), 3.07-2.98 (m, 3H), 2.37 (s, 3H), 2.32-2.09 (m, 4H), 2.05-1.85 (m, 4H). |
| 204 | 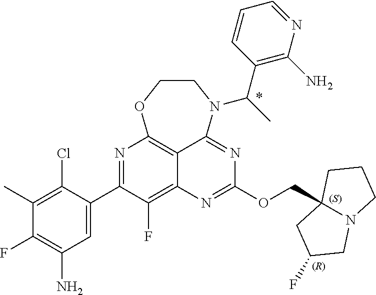 | 641.2 | (400 MHz, CD₃OD) δ 7.98-7.91 (m, 1H), 7.78-7.68 (m, 1H), 6.83-6.73 (m, 2H), 6.65-6.51 (m, 1H), 5.42- 5.19 (m, 1H), 4.49-4.39 (m, 1H), 4.37-4.28 (m, 3H), 3.80-3.68 (m, 1H), 3.57-3.47 m, 1H), 3.30-3.21 (m, 3H), 3.10-2.99 (m, 1H), 2.43-2.14 (m, 6H), 2.07-1.86 (m, 3H), 1.66-1.59 (m, 3H). |
| 205 | 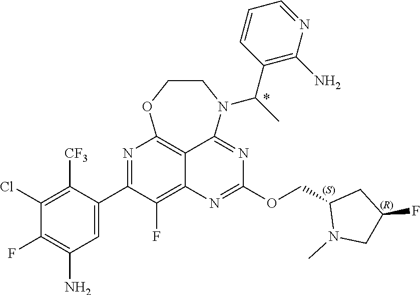 | 669.2 | (400 MHz, CD₃OD) δ 7.98-7.93 (m, 1H), 7.77-7.72 (m, 1H), 6.80-6.75 (m, 1H), 6.70-6.64 (m, 1H), 6.64- 6.56 (m, 1H), 5.27-5.09 (m, 1H), 4.63-4.50 (m, 2H), 4.50-4.24 (m, 2H), 3.77-3.68 (m, 1H), 3.57-3.45 (m, 2H), 3.23-3.14 (m, 1H), 2.75-2.62 (m, 1H), 2.57 (s, 3H), 2.38-2.24 (m, 1H), 2.13-1.92 (m, 1H), 1.68-1.61 (m, 3H). |
| 218 | 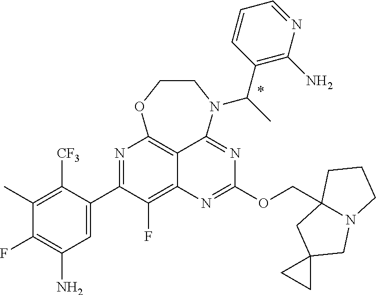 [SMILES: Cc1c(F)c(N)cc(-c2nc3c4c(nc(OCC56CCCN5CC5(CC5)C6)nc4c2F)N(C(C)c2cccnc2N)CCO3)c1C(F)(F)F] | 683.2 | (400 MHz, CD₃OD) δ 7.99-7.90 (m, 1H), 7.78-7.69 (m, 1H), 6.81-6.73 (m, 1H), 6.69-6.54 (m, 2H), 4.53- 4.23 (m, 4H), 3.79-3.65 (m, 1H), 3.58-3.41 (m, 1H), 3.17-3.06 (m, 1H), 2.91-2.75 (m, 3H), 2.44-2.32 (m, 3H), 2.19-2.08 (m, 1H), 2.04-1.82 (m, 5H), 1.70-1.59 (m, 3H), 0.63-0.50 (m, 4H). |
| 221 | 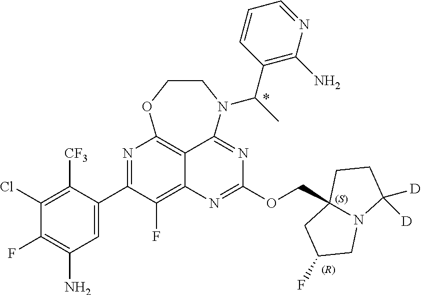 | 697.3 | (400 MHz, CD₃OD) δ 8.00-7.94 (m, 1H), 7.79-7.73 (m, 1H), 6.82-6.76 (m, 1H), 6.72-6.66 (m, 1H), 6.65- 6.56 (m, 1H), 5.45-5.23 (m, 1H), 4.51-4.26 (m, 4H), 3.84-3.70 (m, 1H), 3.59-3.50 (m, 1H), 3.39-3.33 (m, 1H), 3.27-3.22 (m, 1H), 2.43-2.13 (m, 3H), 2.08-1.89 (m, 3H), 1.70-1.62 (m, 3H). |
| 224 | 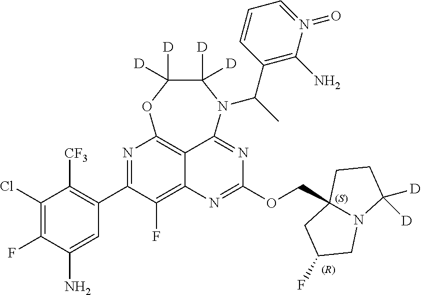 | 717.2 | (400 MHz, CD₃OD) δ 8.10 (d, J = 6.5 Hz, 1H), 7.62 (d, J = 7.8 Hz, 1H), 6.88-6.80 (m, 1H), 6.67 (d, J = 8.1 Hz, 1H), 6.65-6.57 (m, 1H), 5.44-5.16 (m, 1H), 4.38-4.22 (m, 2H), 3.28-3.13 (m, 2H), 2.35-2.08 (m, 3H), 2.03-1.87 (m, 3H), 1.68 (d, J = 6.9 Hz, 3H). |
| 225 | 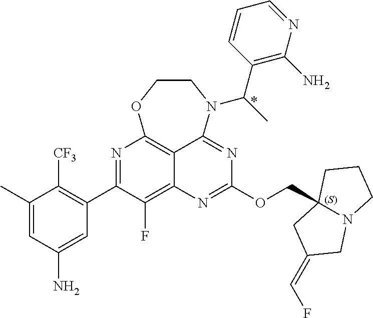 | 669.4 | (400 MHz, CD₃OD) δ 7.96 (d, J = 5.2 Hz, 1H), 7.77 (d, J = 7.6 Hz, 1H), 6.96 (br, 0.5H), 6.81-6.78 (m, 1H), 6.75 (br, 0.5H), 6.68 (s, 1H), 6.57-6.52 (m, 1H), 6.39 (s, 1H), 4.70 (s, 2H), 4.48-4.37 (m, 3H), 4.08 (d, J = 14.8 Hz, 1H), 3.83-3.70 (m, 2H), 3.63-3.55 (m, 1H), 3.25 (t, J = 6.4 Hz, 1H), 2.97 (d, J = 16.0 Hz, 1H), 2.75 (d, J = 16.0 Hz, 1H), 2.41 (d, J = 2.4 Hz, 3H), 2.39-2.33 (m, 1H), 2.28-2.09 (m, 3H), 1.65 (d, J = 6.8 Hz, 3H). |
| 226 | 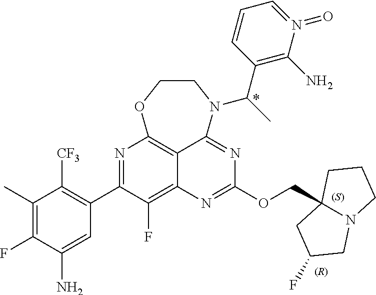 | 691.2 | (400 MHz, CD₃OD) δ 8.13-8.06 (m, 1H), 7.64-7.60 (m, 1H), 6.87-6.80 (m, 1H), 6.65-6.55 (m, 2H), 5.40- 5.23 (m, 1H), 4.52-4.29 (m, 4H), 3.91-3.78 (m, 1H), 3.62-3.54 (m, 1H), 3.29-3.19 (m, 3H), 3.08-3.00 (m, 1H), 2.39-2.12 (m, 6H), 2.05-1.89 (m, 3H), 1.71-1.65 (m, 3H). |
| 227 | 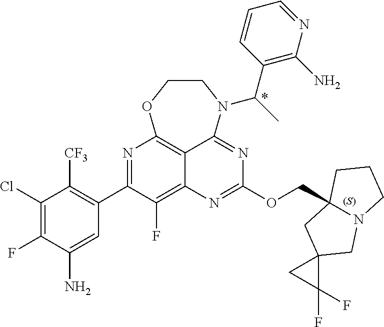 | 739.3 | (400 MHz, CD₃OD) δ 7.95 (d, J = 1.6 Hz, 1H), 7.80- 7.71 (m, 1H), 6.82-6.73 (m, 1H), 6.71-6.54 (m, 2H), 4.51-4.21 (m, 4H), 3.80-3.64 (m, 1H), 3.60-3.42 (m, 1H), 3.28-3.05 (m, 2H), 2.93-2.64 (m, 2H), 2.37-2.24 (m, 1H), 2.21-1.73 (m, 5H), 1.64 (d, J = 6.8 Hz, 3H), 1.51-1.36 (m, 2H). |
| 228 | 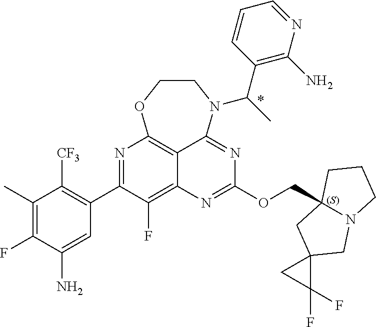 | 719.3 | (400 MHz, CD₃OD) δ 7.97-7.90 (m, 1H), 7.77-7.71 (m, 1H), 6.78-6.73 (m, 1H), 6.67-6.55 (m, 2H), 4.49- 4.25 (m, 4H), 3.79-3.67 (m, 1H), 3.57-3.47 (m, 1H), 3.32-3.21 (m, 1H), 3.21-3.13 (m, 1H), 2.87-2.81 (m, 1H), 2.75-2.65 (m, 1H), 2.41-2.33 (m, 3H), 2.32-2.24 (m, 1H), 2.20-2.13 (m, 1H), 2.04-1.98 (m, 1H), 1.97- 1.74 (m, 3H), 1.64 (d, J = 6.9 Hz, 3H), 1.51-1.37 (m, 2H). |
| 229 | 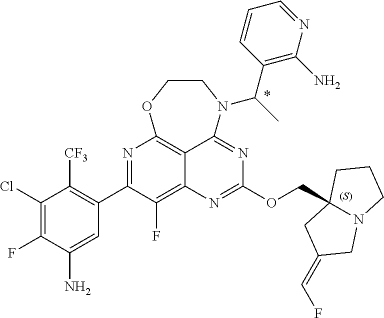 | 707.3 | (400 MHz, CD₃OD) δ 7.94 (d, J = 1.4 Hz, 1H), 7.75 (d, J = 7.3 Hz, 1H), 6.86-6.48 (m, 4H), 4.51-4.23 (m, 4H), 3.91-3.65 (m, 2H), 3.59-3.40 (m, 2H), 3.22-3.10 (m, 1H), 2.82-2.63 (m, 2H), 2.52-2.39 (m, 1H), 2.21- 2.07 (m, 1H), 2.05-1.77 (m, 3H), 1.64 (d, J = 6.9 Hz, 3H). |
| 230 | 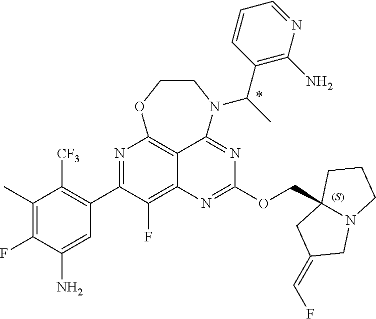 | 687.3 | (400 MHz, CD₃OD) δ 7.97-7.91 (m, 1H), 7.77-7.70 (m, 1H), 6.81-6.51 (m, 4H), 4.48-4.25 (m, 4H), 3.87- 3.80 (m, 1H), 3.79-3.67 (m, 1H), 3.55-3.43 (m, 2H), 3.19-3.11 (m, 1H), 2.77-2.66 (m, 2H), 2.48-2.30 (m, 4H), 2.20-2.11 (m, 1H), 2.03-1.81 (m, 3H), 1.63 (d, J = 6.8 Hz, 3H). |
| 231 | 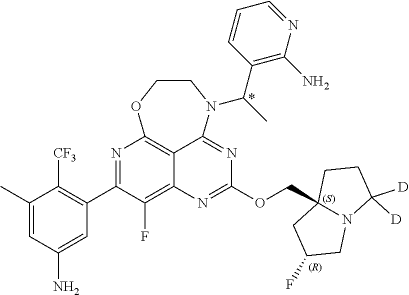 | 659.4 | (400 MHz, CD₃OD) δ 7.98-7.91 (m, 1H), 7.78-7.72 (m, 1H), 6.81-6.73 (m, 1H), 6.70-6.65 (m, 1H), 6.65- 6.52 (m, 1H), 6.38 (s, 1H), 5.40-5.18 (m, 1H), 4.48- 4.25 (m, 4H), 3.80-3.67 (m, 1H), 3.57-3.46 (m, 1H), 3.29-3.14 (m, 2H), 2.44-2.38 (m, 3H), 2.38-2.10 (m, 3H), 2.08-1.85 (m, 3H), 1.68-1.58 (m, 3H). |
| 232 | 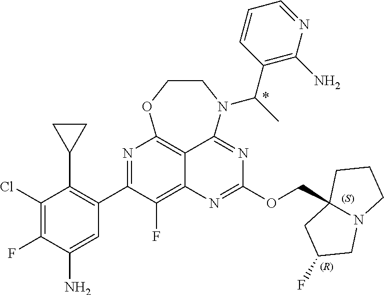 | 667.3 | (400 MHz, CD₃OD) δ 7.98-7.91 (m, 1H), 7.77-7.72 (m, 1H), 6.80-6.73 (m, 2H), 6.64-6.56 (m, 1H), 5.41- 5.20 (m, 1H), 4.48-4.27 (m, 4H), 3.81-3.68 (m, 1H), 3.58-3.48 (m, 1H), 3.27-3.17 (m, 2H), 3.05-2.98 (m, 1H), 2.39-2.12 (m, 3H), 2.04-1.80 (m, 4H), 1.64 (d, J = 6.9 Hz, 3H), 0.76-0.54 (m, 2H), 0.20-0.03 (m, 2H). |
| 233 | 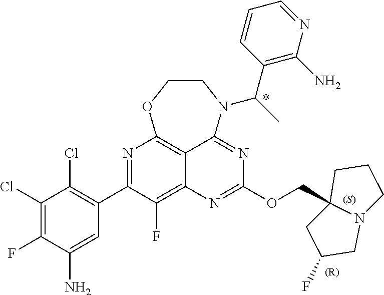 | 661.2 | (400 MHz, CD₃OD) δ 7.97-7.90 (m, 1H), 7.78-7.61 (m, 1H), 6.88-6.82 (m, 1H), 6.79-6.72 (m, 1H), 6.64- 6.51 (m, 1H), 5.43-5.18 (m, 1H), 4.50-4.24 (m, 4H), 3.81-3.68 (m, 1H), 3.58-3.47 (m, 1H), 3.28-3.13 (m, 3H), 3.05-2.94 (m, 1H), 2.40-2.12 (m, 3H), 2.04-1.89 (m, 3H), 1.69-1.59 (m, 3H). |
| 237 | 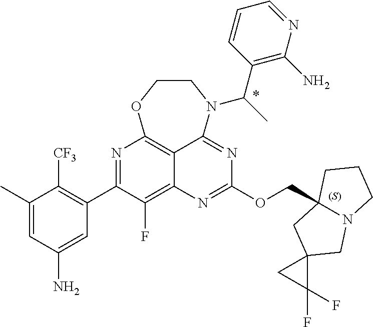 | 701.3 | (400 MHz, CD₃OD) δ 7.98-7.91 (m, 1H), 7.79-7.73 (m, 1H), 6.80-6.73 (m, 1H), 6.70-6.56 (m, 2H), 6.39 (s, 1H), 4.44-4.22 (m, 4H), 3.79-3.64 (m, 1H), 3.55- 3.45 (m, 1H), 3.25-3.00 (m, 2H), 2.91-2.69 (m, 2H), 2.48-2.34 (m, 3H), 2.34-2.23 (m, 1H), 2.19-1.87 (m, 5H), 1.69-1.61 (m, 3H), 1.48-1.40 (m, 2H). |
| 243 | 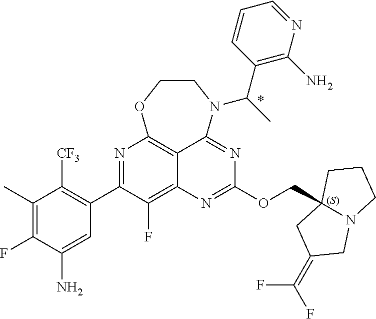 | 705.6 | (400 MHz, CD₃OD) δ 7.99-7.91 (m, 1H), 7.78-7.71 (m, 1H), 6.80-6.73 (m, 1H), 6.64-6.49 (m, 2H), 4.48- 4.26 (m, 4H), 3.83-3.69 (m, 2H), 3.57-3.47 (m, 1H), 3.45-3.37 (m, 1H), 3.18-3.09 (m, 1H), 2.84-2.76 (m, 1H), 2.74-2.62 (m, 1H), 2.56-2.46 (m, 1H), 2.40-2.33 (m, 3H), 2.20-2.09 (m, 1H), 2.00-1.84 (m, 3H), 1.69- 1.58 (m, 3H). |
| 262 | 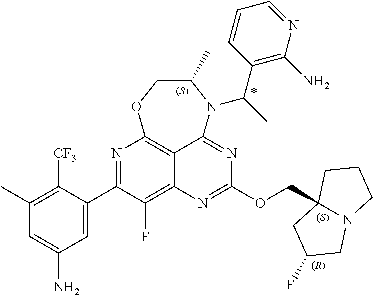 | 671.2 | (400 MHz, CD₃OD) δ 7.98-7.92 (m, 1H), 7.84-7.76 (m, 1H), 6.82-6.75 (m, 1H), 6.73-6.61 (m, 2H), 6.51- 6.32 (m, 1H), 5.45-5.15 (m, 1H), 4.41-4.20 (m, 3H), 4.07-3.91 (m, 2H), 3.27-3.14 (m, 3H), 3.06-2.93 (m, 1H), 2.44-2.08 (m, 6H), 2.02-1.83 (m, 3H), 1.73-1.66 (m, 3H), 1.31-1.21 (m, 3H). |
| 266 | 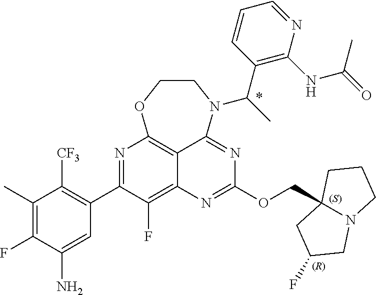 | 717.3 | (400 MHz, CD₃OD) δ 8.49-8.39 (m, 1H), 8.20-8.06 (m, 1H), 7.57-7.41 (m, 1H), 6.96-6.77 (m, 1H), 6.66- 6.47 (m, 1H), 5.42-5.09 (m, 1H), 4.74-4.05 (m, 5H), 3.69-3.51 (m, 1H), 3.35-3.30 (m, 1H), 3.24-3.15 (m, 2H), 3.04-2.97 (m, 1H), 2.38-2.34 (m, 3H), 2.34-2.08 (m, 3H), 2.04-1.88 (m, 3H), 1.75-1.62 (m, 6H). |
| 267 | 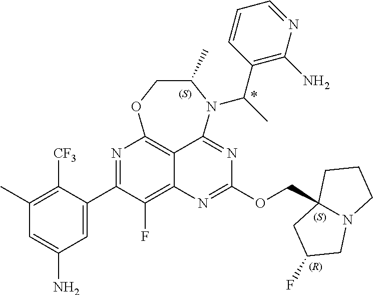 | 671.3 | (400 MHz, CD₃OD) δ 7.98-7.92 (m, 1H), 7.81-7.75 (m, 1H), 6.82-6.69 (m, 2H), 6.68-6.64 (m, 1H), 6.49- 6.26 (m, 1H), 5.42-5.15 (m, 1H), 4.64-4.50 (m, 1H), 4.38-4.21 (m, 3H), 4.11-3.97 (m, 1H), 3.28-3.15 (m, 3H), 3.07-2.96 (m, 1H), 2.48-2.10 (m, 6H), 2.05-1.84 (m, 3H), 1.72-1.59 (m 3H), 0.70-0.59 (m, 3H). |
| 268 | 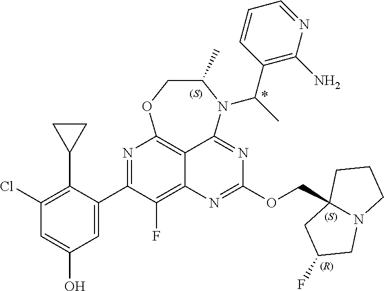 | 664.3 | (400 MHz, CD₃OD) δ 7.99-7.92 (m, 1H), 7.83-7.75 (m, 1H), 6.91 (d, J = 2.6 Hz, 1H), 6.80-6.69 (m, 3H), 5.45-5.17 (m, 1H), 4.67-4.55 (m, 1H), 4.38-4.23 (m, 3H), 4.15-3.96 (m, 1H), 3.27-3.14 (m, 3H), 3.09-2.93 (m, 1H), 2.42-2.11 (m, 3H), 2.01-1.78 (m, 4H), 1.72- 1.62 (m, 3H), 0.71-0.65 (m, 3H), 0.65-0.55 (m, 2H), 0.19-0.00 (m, 2H). |
| 281 | 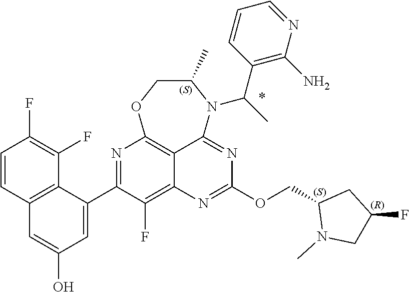 | 650.3 | (400 MHz, CD₃OD) δ 8.03-7.92 (m, 1H), 7.83-7.75 (m, 1H), 7.62-7.49 (m, 1H), 7.42-7.31 (m, 1H), 7.30- 7.12 (m, 2H), 6.84-6.70 (m, 2H), 5.24-5.10 (m, 1H), 4.62-4.52 (m, 3H), 4.37-4.28 (m, 1H), 4.15-4.01 (m, 1H), 3.58-3.43 (m, 1H), 3.23-3.14 (m, 1H), 2.75-2.48 (m, 4H), 2.38-2.16 (m, 1H), 2.12-1.93 (m, 1H), 1.67 (d, J = 6.8 Hz, 3H), 0.81-0.63 (m, 3H). |
| 289 | 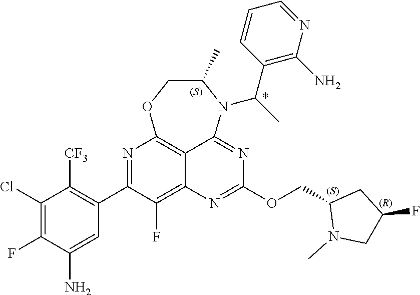 | 683.3 | (400 MHz, CD₃OD) δ 8.44-8.27 (m, 1H), 8.11-7.93 (m, 1H), 7.21-7.02 (m, 1H), 6.88-6.56 (m, 2H), 5.66- 5.32 (m, 1H), 5.16-4.91 (m, 2H), 4.75-4.66 (m, 1H), 4.53-4.00 (m, 4H), 3.72-3.53 (m, 1H), 3.21 (s, 3H), 2.79-2.60 (m, 1H), 2.59-2.31 (m, 1H), 1.92-1.65 (m, 3H), 0.94-0.67 (m, 3H). |
| 290 | 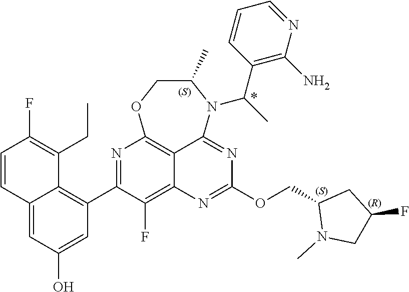 | 660.3 | (400 MHz, CD₃OD) δ 8.02-7.93 (m, 1H), 7.85-7.75 (m, 1H), 7.70-7.57 (m, 1H), 7.32-6.95 (m, 3H), 6.86- 6.66 (m, 2H), 5.27-5.08 (m, 1H), 4.60-4.51 (m, 3H), 4.41-4.27 (m, 1H), 4.15-3.99 (m, 1H), 3.60-3.43 (m, 1H), 3.26-3.14 (m, 1H), 2.75-2.56 (m, 4H), 2.50-2.23 (m, 3H), 2.14-1.94 (m, 1H), 1.74-1.63 (m, 3H), 0.92- 0.75 (m, 3H), 0.74-0.64 (m, 3H). |
| 291 | 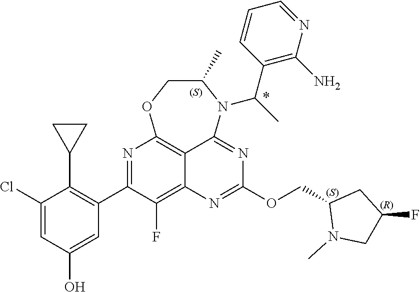 | 638.3 | (400 MHz, CD₃OD) δ 8.00-7.93 (m, 1H), 7.84-7.77 (m, 1H), 6.94-6.90 (m, 1H), 6.81-6.72 (m, 3H), 5.31- 5.06 (m, 1H), 4.64-4.54 (m, 3H), 4.37-4.26 (m, 1H), 4.11-4.00 (m, 1H), 3.56-3.44 (m, 1H), 3.20-3.11 (m, 1H), 2.73-2.59 (m, 1H), 2.57-2.52 (m, 3H), 2.37-2.23 (m, 1H), 2.10-1.95 (m, 1H), 1.89-1.81 (m, 1H), 1.67 (d, J = 6.8 Hz, 3H), 0.69 (d, J = 6.9 Hz, 3H), 0.66- 0.58 (m, 2H), 0.17-0.05 (m, 2H). |
| 297 | 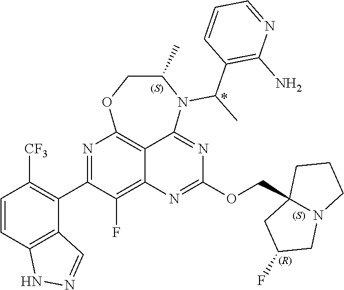 | 682.3 | (400 MHz, CD₃OD) δ 8.01-7.93 (m, 1H), 7.89-7.75 (m, 4H), 6.83-6.72 (m, 2H), 5.40-5.20 (m, 1H), 4.65- 4.58 (m, 1H), 4.39-4.28 (m, 3H), 4.13-4.04 (m, 1H), 3.25-3.11 (m, 3H), 3.04-2.95 (m, 1H), 2.32-2.10 (m, 3H), 2.00-1.84 (m, 3H), 1.70-1.60 (m, 3H), 0.77-0.66 (m, 3H). |
| 298 | 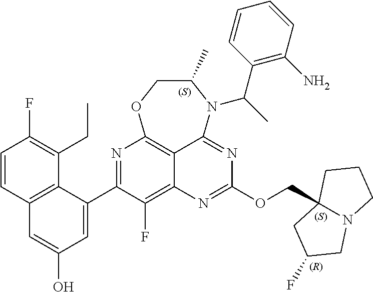 | 685.3 | (400 MHz, CD₃OD) δ 8.77-8.57 (m, 1H), 7.67-7.54 (m, 1H), 7.41-7.31 (m, 1H), 7.25-7.16 (m, 3H), 7.12- 6.98 (m, 2H), 5.44-5.13 (m, 1H), 4.77-4.68 (m, 1H), 4.55-4.44 (m, 1H), 4.37-4.24 (m, 2H), 3.93-3.80 (m, 1H), 3.63-3.44 (m, 1H), 3.27-3.10 (m, 3H), 3.05-2.92 (m, 1H), 2.64-2.49 (m, 1H), 2.41-2.30 (m, 1H), 2.28- 2.10 (m, 3H), 2.03-1.82 (m, 3H), 1.46-1.37 (m, 3H), 1.21-1.10 (m, 3H), 0.91-0.77 (m, 3H). |
| 300 | 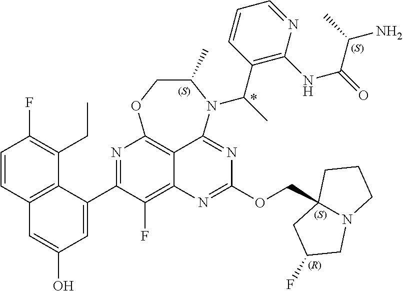 | 757.2 | (400 MHz, DMSO-d₆) δ 11.10-10.87 (m, 1H), 10.31- 9.87 (m, 1H), 8.58-8.44 (m, 1H), 8.25-8.12 (m, 3H), 7.76-7.68 (m, 1H), 7.55-7.43 (m, 1H), 7.37-7.27 (m, 2H), 7.07-6.86 (m, 1H), 6.63-6.42 (m, 1H), 5.70-5.43 (m, 1H), 4.59-4.49 (m, 3H), 3.90-3.70 (m, 6H), 3.35- 3.26 (m, 1H), 2.63-2.53 (m, 1H), 2.44-1.91 (m, 7H), 1.71-1.59 (m, 3H), 1.38-1.17 (m, 2H), 1.12-0.90 (m, 2H), 0.85-0.63 (m, 4H), 0.56-0.35 (m, 3H). |
| 302 | 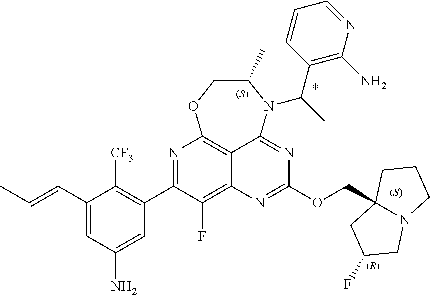 | 697.3 | (400 MHz, CD₃OD) δ 7.97-7.91 (m, 1H), 7.81-7.75 (m, 1H), 7.39-7.34 (m, 1H), 7.32-7.25 (m, 1H), 6.79- 6.68 (m, 3H), 6.65-6.58 (m, 1H), 5.43-5.16 (m, 1H), 4.65-4.53 (m, 1H), 4.36-4.21 (m, 3H), 4.09-3.95 (m, 1H), 3.27-3.12 (m, 3H), 3.04-2.93 (m, 1H), 2.36-2.13 (m, 3H), 2.10 (s, 3H), 2.02-1.86 (m, 3H), 1.64 (d, J = 6.9 Hz, 3H), 0.67 (d, J = 6.9 Hz, 3H). |
| 305 | 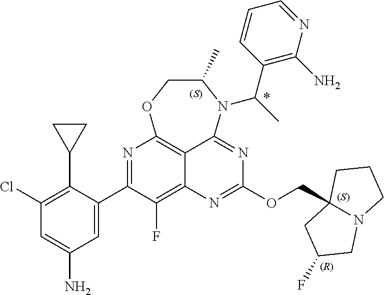 | 663.3 | (400 MHz, CD₃OD) δ 8.01-7.92 (m, 1H), 7.83-7.75 m, 1H), 6.83-6.80 (m, 1H), 6.79-6.71 (m, 2H), 6.63- 6.60 (m, 1H), 5.41-5.16 (m, 1H), 4.66-4.57 (m, 1H), 4.33-4.27 (m, 3H), 4.13-3.98 (m, 1H), 3.26-3.15 (m, 3H), 3.06-2.95 (m, 1H), 2.37-2.15 (m, 3H), 2.04-1.91 (m, 3H), 1.83-1.74 (m, 1H), 1.72-1.62 (m, 3H), 0.71- 0.65 (m, 3H), 0.64-0.52 (m, 2H), 0.13-0.01 (m, 2H). |
| 306 | 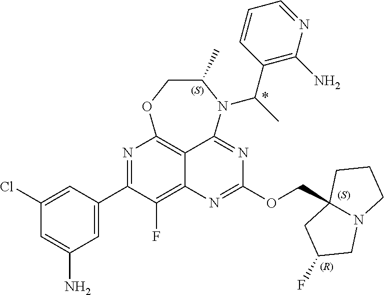 | 623.3 | (400 MHz, CD₃OD) δ 7.98-7.91 (m, 1H), 7.82-7.74 (m, 1H), 7.24-7.15 (m, 2H), 6.81-6.64 (m, 3H), 5.43- 5.18 (m, 1H), 4.65-4.55 (m, 1H), 4.35-4.24 (m, 3H), 4.10-3.97 (m, 1H), 3.26-3.14 (m, 3H), 3.03-2.95 (m, 1H), 2.37-2.12 (m, 3H), 2.01-1.88 (m, 3H), 1.70-1.61 (m, 3H), 0.74-0.62 (m, 3H). |
| 311 | 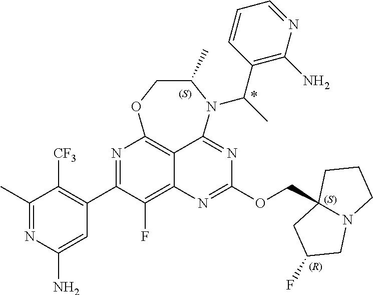 | 672.3 | (400 MHz, CD₃OD) δ 7.99-7.93 (m, 1H), 7.81-7.76 (m, 1H), 6.84-6.69 (m, 2H), 6.43-6.26 (m, 1H), 5.44- 5.17 (m, 1H), 4.65-4.56 (m, 1H), 4.38-4.24 (m, 3H), 4.09-3.99 (m, 1H), 3.27-3.19 (m, 2H), 3.18-3.14 (m, 1H), 3.05-2.95 (m, 1H), 2.56-2.49 (m, 3H), 2.38-2.12 (m, 3H), 2.02-1.85 (m, 3H), 1.66 (d, J = 6.8 Hz, 3H), 0.67 (d, J = 6.9 Hz, 3H). |
| 312 | 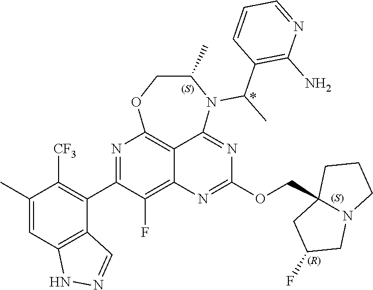 | 696.2 | (400 MHz, CD₃OD) δ 8.01-7.92 (m, 1H), 7.84-7.78 (m, 1H), 7.75-7.57 (m, 2H), 6.84-6.72 (m, 2H), 5.37- 5.15 (m, 1H), 4.69-4.57 (m, 1H), 4.38-4.30 (m, 3H), 4.14-4.02 (m, 1H), 3.27-3.19 (m, 2H), 3.17-3.13 (m, 1H), 3.06-2.96 (m, 1H), 2.70-2.62 (m, 3H), 2.36-2.12 (m, 3H), 2.01-1.86 (m, 3H), 1.68 (d, J = 6.8 Hz, 3H), 0.76-0.68 (m, 3H). |
| 313 | 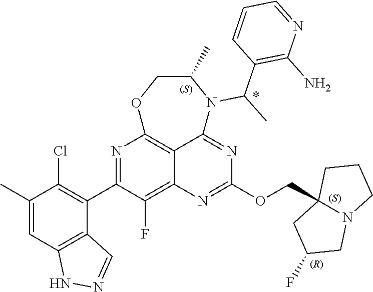 | 662.3 | (400 MHz, CD₃OD) δ 8.01-7.91 (m, 1H), 7.82-7.57 (m, 3H), 6.84-6.69 (m, 2H), 5.43-5.16 (m, 1H), 4.69- 4.56 (m, 1H), 4.40-4.27 (m, 3H), 4.12-4.02 (m, 1H), 3.27-3.09 (m, 3H), 3.04-2.95 (m, 1H), 2.60-2.49 (m, 3H), 2.41-2.10 (m, 3H), 2.04-1.84 (m, 3H), 1.72-1.62 (m, 3H), 0.79-0.64 (m, 3H). |
| 340 | 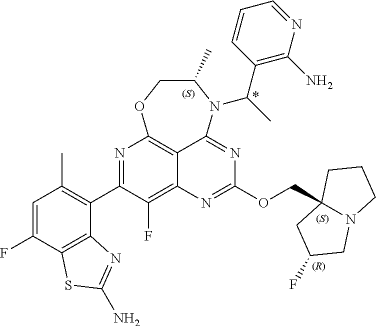 | 678.3 | (400 MHz, CD₃OD) δ 8.00-7.93 (m, 1H), 7.85-7.76 (m, 1H), 6.90-6.82 (m, 1H), 6.82-6.72 (m, 2H), 5.40- 5.21 (m, 1H), 4.64-4.58 (m, 1H), 4.37-4.27 (m, 3H), 4.16-4.00 (m, 1H), 3.28-2.98 (m, 4H), 2.35-2.14 (m, 6H), 2.03-1.88 (m, 3H), 1.72-1.66 (m, 3H), 0.82-0.62 (m, 3H). |
| 361 | 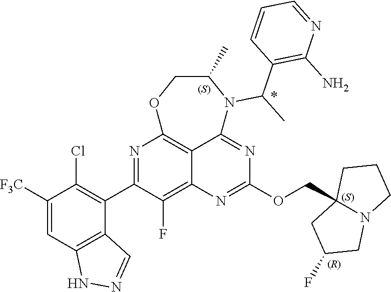 | 716.3 | (400 MHz, CD₃OD) δ 8.18 (s, 1H), 8.02-7.78 (m, 3H), 6.86-6.68 (m, 2H), 5.40-5.20 (m, 1H), 4.68-4.60 (m, 1H), 4.45-4.23 (m, 3H), 4.16-4.00 (m, 1H), 3.28- 2.98 (m, 4H), 2.40-2.14 (m, 3H), 2.04-1.90 (m, 3H), 1.75-1.62 (m, 3H), 0.82-0.66 (m, 3H). |
| 395 | 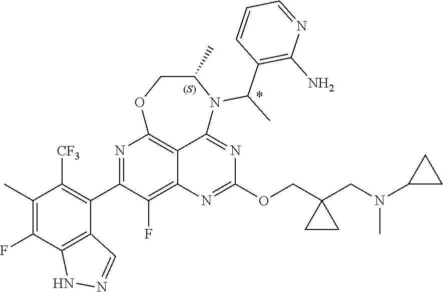 | 709.8 | (400 MHz, CD₃OD) δ 8.03-7.91 (m, 1H), 7.91-7.67 (m, 2H), 6.87-6.69 (m, 2H), 4.70-4.57 (m, 1H), 4.55- 4.41 (m, 1H), 4.41-4.24 (m, 2H), 4.16-3.98 (m, 1H), 2.75-2.47 (m, 5H), 2.39 (s, 3H), 1.83-1.54 (m, 4H), 0.86-0.59 (m, 5H), 0.59-0.27 (m, 6H). |
| 396 | 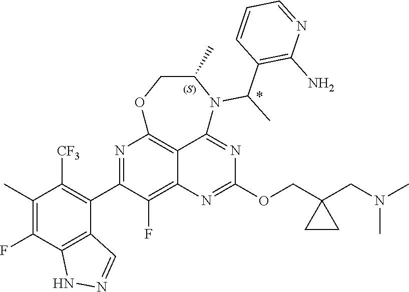 | 683.7 | (400 MHz, CD₃OD) δ 8.04-7.91 (m, 1H), 7.89-7.64 (m, 2H), 6.95-6.65 (m, 2H), 4.67-4.54 (m, 1H), 4.50- 4.26 (m, 3H), 4.17-3.96 (m, 1H), 2.71-2.18 (m, 11H), 1.69 (d, J = 6.8 Hz, 3H), 0.88-0.62 (m, 5H), 0.58- 0.50 (m, 2H). |
| 397 | 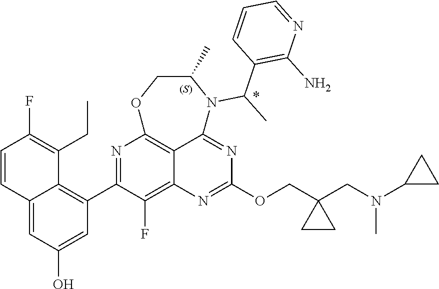 | 681.8 | (400 MHz, CD₃OD) δ 8.03-7.93 (m, 1H), 7.86-7.77 (m, 1H), 7.68-7.59 (m, 1H), 7.28-7.18 (m, 2H), 7.14- 6.98 (m, 1H), 6.81-6.71 (m, 2H), 4.66-4.56 (m, 1H), 4.49-4.42 (m, 1H), 4.36-4.27 (m, 2H), 4.11-4.02 (m, 1H), 2.68-2.24 (m, 7H), 1.72-1.59 (m, 4H), 0.96-0.78 (m, 3H), 0.77-0.66 (m, 5H), 0.57-0.47 (m, 2H), 0.47- 0.27 (m, 4H). |

| Com- | | Catalyst, alkali and |
|---|---|---|
| pound | Intermediate | solvent used in step 1 |
| 5 | I-A2 | cataCXium A Pd-G₃, |
| | 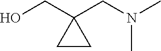 | K₃PO₄, THF, H₂O |
| | I-B2 | |
| | I-C3 | |
| 6 | I-A1 | cataCXium A Pd-G₃, |
| | I-B3 | K₃PO₄, DMA, H₂O |
| | I-C3 | |
| 7 | I-A1 | cataCXium A Pd-G₃, |
| | I-B2 | K₃PO₄, DMA, H₂O |
| | I-C3 | |
| 8 | I-A1 | cataCXium A Pd-G₃, |
| | I-B4 | K₃PO₄, DMA, H₂O |
| | I-C3 | |
| 9 | I-A1 | cataCXium A Pd-G₃, |
| | I-B4 | K₃PO₄, DMA, H₂O |
| | I-C3 | |
| 10 | I-A1 | cataCXium A Pd-G₃, |
| | EtONa | K₃PO₄, DMA, H₂O |
| | I-C3 | |
| 11 | I-A1 | cataCXium A Pd-G₃, |
| | 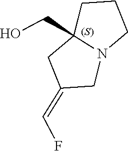 | K₃PO₄, DMA, H₂O |
| | I-B5 | |
| | I-C3 | |
| 12 | I-A1 | cataCXium A Pd-G₃, |
| | 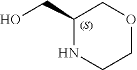 | K₃PO₄, DMA, H₂O |
| | I-B6 | |
| | I-C3 | |
| 13 | I-A1 | cataCXium A Pd-G₃, |
| | 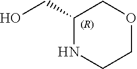 | K₃PO₄, DMA, H₂O |
| | I-B7 | |
| | I-C3 | |
| 14 | I-A2 | cataCXium A Pd-G₃, |
| | I-B4 | K₃PO₄, THF, H₂O |
| | I-C3 | |
| 16 | I-A3 | cataCXium A Pd-G₃, |
| | I-B4 | K₃PO₄, DMA, H₂O |
| | I-C3 | |
| 17 | I-A5 | cataCXium A Pd-G₃, |
| | I-B4 | K₃PO₄, DMA, H₂O |
| | I-C3 | |
| 19 | 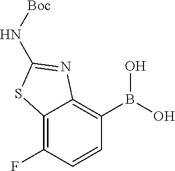 [SMILES: CC(C)(C)OC(=O)Nc1nc2c(B(O)O)ccc(F)c2s1] | cataCXium A Pd-G₃, K₃PO₄, DMA, H₂O |
| | I-A7 | |
| | I-B4 | |
| | I-C3 | |
| 22 | I-A1 | cataCXium A Pd-G₃, |
| | I-C4 | K₃PO₄, MeCN, H₂O |
| 33 | I-A1 | cataCXium A Pd-G₃, |
| | I-C3 | K₃PO₄, DMA, H₂O |
| 45 | I-A8 | cataCXium A Pd-G₃, |
| | I-B8 | K₃PO₄, DMA, H₂O |
| | I-C3 | |
| 46 | I-A1 | cataCXium A Pd-G₃, |
| | I-B8 | K₃PO₄, DMA, H₂O |
| | I-C3 | |
| 47 | I-A10 | cataCXium A Pd-G₃, |
| | I-B1 | K₃PO₄, DMA, H₂O |
| | I-C3 | |
| 65 | I-A12 | cataCXium A Pd-G₃, |
| | I-B3 | K₃PO₄, DMA, H₂O |
| | I-C3 | |
| 67 | I-A15 | cataCXium A Pd-G₃, |
| | I-B3 | K₃PO₄, DMA, H₂O |
| | I-C3 | |
| 68 | I-A15 | cataCXium A Pd-G₃, |
| | 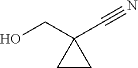 | K₃PO₄, DMA, H₂O |
| | I-B9 | |
| | I-C3 | |
| 69 | I-A12 | cataCXium A Pd-G₃, |
| | I-B2 | K₃PO₄, DMA, H₂O |
| | I-C3 | |
| 70 | I-A12 | cataCXium A Pd-G₃, |
| | I-B8 | K₃PO₄, DMA, H₂O |
| | I-C3 | |
| 73 | I-A12 | cataCXium A Pd-G₃, |
| | I-B10 | K₃PO₄, DMA, H₂O |
| | I-C3 | |
| 82 | I-A16 | cataCXium A Pd-G₃, K₃PO₄, DMA, H₂O |
| | I-B1 | |
| | I-C3 | |
| 83 | I-A17 | cataCXium A Pd-G₃, K₃PO₄, DMA, H₂O |
| | I-B1 | |
| | I-C3 | |
| 87 | I-A12 | cataCXium A Pd-G₃, |
| | 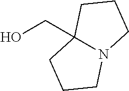 | K₃PO₄, DMA, H₂O |
| | I-B12 | |
| | I-C3 | |
| 94 | I-A19 | cataCXium A Pd-G₃, K₃PO₄, THF, H₂O |
| | I-B3 | |
| | I-C3 | |
| 97 | I-A17 | cataCXium A Pd-G₃, K₃PO₄, DMA, H₂O |
| | I-B3 | |
| | I-C3 | |
| 104 | I-A18 | Pd(dppf)Cl₂•CH₂Cl₂, K₂CO₃, DMA, H₂O |
| | I-B15 | |
| | I-C3 | |
| 105 | I-A12 | cataCXium A Pd-G₃, K₃PO₄, DMA, H₂O |
| | I-B16 | |
| | I-C3 | |
| 106 | I-A12 | cataCXium A Pd-G₃, K₃PO₄, DMA, H₂O |
| 107 | 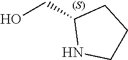 | |
| | I-B17 | |
| | I-C3 | |
| 108 | I-A12 | cataCXium A Pd-G₃, K₃PO₄, DMA, H₂O |
| | I-B18 | |
| | I-C3 | |
| 111 | I-A14 | cataCXium A Pd-G₃, K₃PO₄, DMA, H₂O |
| | I-B13 | |
| | I-C3 | |
| 112 | I-A14 | cataCXium A Pd-G₃, K₃PO₄, DMA, H₂O |
| | I-B15 | |
| | I-C3 | |
| 113 | I-A14 | cataCXium A Pd-G₃, K₃PO₄, DMA, H₂O |
| | I-B16 | |
| | I-C3 | |
| 114 | I-A12 | cataCXium A Pd-G₃, K₃PO₄, DMA, H₂O |
| | 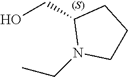 | |
| | I-B20 | |
| | I-C3 | |
| 119 | I-A18 | Pd(dppf)Cl₂•CH₂Cl₂, K₂CO₃, DMA, H₂O |
| | I-B16 | |
| | I-C3 | |
| 120 | I-A18 | Pd(dppf)Cl₂•CH₂Cl₂, K₂CO₃, DMA, H₂O |
| | I-B1 | |
| | I-C3 | |
| 121 | I-A12 | cataCXium A Pd-G₃, K₃PO₄, DMA, H₂O |
| | I-B1 | |
| | I-C5 | |
| 122 | I-A12 | cataCXium A Pd-G₃, K₃PO₄, DMA, H₂O |
| | I-B22 | |
| | I-C3 | |
| 123 | I-A12 | cataCXium A Pd-G₃, K₃PO₄, DMA, H₂O |
| | I-B23 | |
| | I-C3 | |
| 126 | I-A6 | cataCXium A Pd-G₃, K₃PO₄, DMA, H₂O |
| | I-B22 | |
| | I-C3 | |
| 129 | I-A6 | cataCXium A Pd-G₃, K₃PO₄, DMA, H₂O |
| | I-B16 | |
| | I-C3 | |
| 134 | I-A3 | cataCXium A Pd-G₃, K₃PO₄, DMA, H₂O |
| | 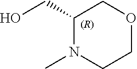 | |
| | I-B24 | |
| | I-C3 | |
| 135 | I-A20 | cataCXium A Pd-G₃, K₃PO₄, DMA, H₂O |
| | I-B1 | |
| | I-C3 | |
| 136 | I-A20 | cataCXium A Pd-G₃, K₃PO₄, DMA, H₂O |
| | I-B22 | |
| | I-C3 | |
| 139 | I-A3 | cataCXium A Pd-G₃, K₃PO₄, DMA, H₂O |
| | 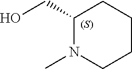 | |
| | I-B25 | |
| | I-C3 | |
| 140 | I-A12 | cataCXium A Pd-G₃, K₃PO₄, DMA, H₂O |
| | I-B1 | |
| | I-C6 | |
| 141 | I-A12 | cataCXium A Pd-G₃, K₃PO₄, DMA, H₂O |
| | I-B1 | |
| | I-C7 | |
| 146 | I-A21 | cataCXium A Pd-G₃, K₃PO₄, DMA, H₂O |
| | I-B1 | |
| | I-C3 | |
| 148 | I-A18 | Pd(dppf)Cl₂•CH₂Cl₂, K₂CO₃, DMA, H₂O |
| | I-B26 | |
| | I-C9 | |
| 149 | I-A18 | Pd(dppf)Cl₂•CH₂Cl₂, K₂CO₃, DMA, H₂O |
| | I-B22 | |
| | I-C9 | |
| 150 | I-A18 | Pd(dppf)Cl₂•CH₂Cl₂, K₂CO₃, DMA, H₂O |
| | I-B27 | |
| | I-C9 | |
| 157 | I-A18 | Pd(dppf)Cl₂•CH₂Cl₂, K₂CO₃, DMA, H₂O |
| | I-B1 | |
| | I-C6 | |
| 158 | I-A25 | cataCXium A Pd-G₃, K₃PO₄, DMA, H₂O |
| | I-B1 | |
| | I-C9 | |
| 159 | I-A26 | cataCXium A Pd-G₃, K₃PO₄, DMA, H₂O |
| | I-B1 | |
| | I-C3 | |
| 163 | I-A18 | Pd(dppf)Cl₂•CH₂Cl₂, K₂CO₃, DMA, H₂O |
| | 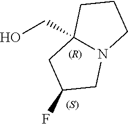 | |
| | I-B28 | |
| | I-C9 | |
| 164 | I-A18 | Pd(dppf)Cl₂•CH₂Cl₂, K₂CO₃, DMA, H₂O |
| | 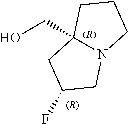 | |
| | I-B29 | |
| | I-C9 | |
| 165 | I-A18 | Pd(dppf)Cl₂•CH₂Cl₂, K₂CO₃, DMA, H₂O |
| | 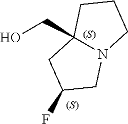 | |
| | I-B30 | |
| | I-C9 | |
| 168 | I-A28 | Pd(PPh₃)₂Cl₂, K₂CO₃, DMA, H₂O |
| | I-B1 | |
| | I-C9 | |
| 171 | I-A18 | Pd(dppf)Cl₂•CH₂Cl₂, K₂CO₃, DMA, H₂O |
| | I-B32 | |
| | I-C9 | |
| 173 | I-A12 | cataCXium A Pd-G₃, K₃PO₄, DMA, H₂O |
| | I-B1 | |
| | I-C11 | |
| 174 | I-A29 | cataCXium A Pd-G₃, K₃PO₄, DMA, H₂O |
| | I-B1 | |
| | I-C9 | |
| 177 | I-A18 | Pd(dppf)Cl₂•CH₂Cl₂, K₂CO₃, DMA, H₂O |
| | I-B1 | |
| | I-C11 | |
| 180 | I-A18 | Pd(dppf)Cl₂•CH₂Cl₂, K₂CO₃, DMA, H₂O |
| | I-B1 | |
| | I-C12 | |
| 181 | I-A12 | Pd(dppf)Cl₂•CH₂Cl₂, K₂CO₃, DMA, H₂O |
| | I-B1 | |
| | I-C13 | |
| 185 | I-A18 | Pd(dppf)Cl₂•CH₂Cl₂, K₂CO₃, DMA, H₂O |
| | I-B10 | |
| | I-C9 | |
| 187 | I-A18 | Pd(dppf)Cl₂•CH₂Cl₂, K₂CO₃, DMA, H₂O |
| | I-B1 | |
| | I-C14 | |
| 195 | I-A18 | Pd(dppf)Cl₂•CH₂Cl₂, K₂CO₃, DMA, H₂O |
| | I-B34 | |
| | I-C9 | |
| 196 | I-A18 | Pd(dppf)Cl₂•CH₂Cl₂, K₂CO₃, DMA, H₂O |
| | I-B1 | |
| | I-C15 | |
| 197 | 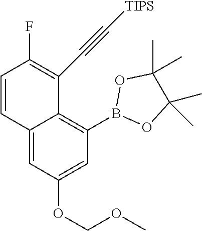 | Pd(dppf)Cl₂•CH₂Cl₂, K₂CO₃, DMA, H₂O |
| | I-A30 | |
| | I-B1 | |
| | I-C15 | |
| 198 | I-A18 | Pd(dppf)Cl₂•CH₂Cl₂, K₂CO₃, DMA, H₂O |
| | I-B1 | |
| | I-C16 | |
| 204 | I-A32 | Pd(dppf)Cl₂•CH₂Cl₂, K₂CO₃, DMA, H₂O |
| | I-B1 | |
| | I-C9 | |
| 205 | I-A28 | Pd(dppf)Cl₂•CH₂Cl₂, K₂CO₃, DMA, H₂O |
| | I-B10 | |
| | I-C9 | |
| 218 | I-A18 | Pd(dppf)Cl₂•CH₂Cl₂, |
| | 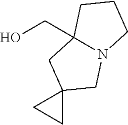 [SMILES: OCC12CCCN1CC1(CC1)C2] | K₂CO₃, DMA, H₂O |
| | I-B35 | |
| | I-C9 | |
| 221 | I-A28 | Pd(dppf)Cl₂•CH₂Cl₂, K₂CO₃, DMA, H₂O |
| | I-B27 | |
| | I-C9 | |
| 224 | I-A28 | Pd(dppf)Cl₂•CH₂Cl₂, K₂CO₃, DMA, H₂O |
| | I-B27 | A by-product obtained |
| | I-C17 | in the oxidation step |
| | | of compounds 222 |
| I-C17 | | and 223 preparation |
| 225 | I-A12 | Pd(dppf)Cl₂•CH₂Cl₂, |
| | I-B5 | K₂CO₃, DMA, H₂O |
| | I-C9 | |
| 226 | I-A18 | Pd(dppf)Cl₂•CH₂Cl₂, |
| | I-B1 | K₂CO₃, DMA, H₂O |
| | I-C9 | A by-product obtained |
| | | in the oxidation step |
| | | of compound 137 |
| | | preparation |
| 227 | I-A28 | Pd(dppf)Cl₂•CH₂Cl₂, |
| | 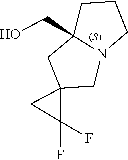 | K₂CO₃, DMA, H₂O |
| | I-B37 | |
| | I-C9 | |
| 228 | I-A18 | Pd(dppf)Cl₂•CH₂Cl₂, K₂CO₃, DMA, H₂O |
| | I-B37 | |
| | I-C9 | |
| 229 | I-A28 | Pd(dppf)Cl₂•CH₂Cl₂, K₂CO₃, DMA, H₂O |
| | I-B5 | |
| | I-C9 | |
| 230 | I-A18 | Pd(dppf)Cl₂•CH₂Cl₂, K₂CO₃, DMA, H₂O |
| | I-B5 | |
| | I-C9 | |
| 231 | I-A12 | Pd(dppf)Cl₂•CH₂Cl₂, K₂CO₃, DMA, H₂O |
| | I-B27 | |
| | I-C9 | |
| 232 | I-A33 | cataCXium A Pd-G₃, K₃PO₄, DMA, H₂O |
| | I-B1 | |
| | I-C9 | |
| 233 | I-A34 | Pd(dppf)Cl₂•CH₂Cl₂, K₂CO₃, DMA, H₂O |
| | I-B1 | |
| | I-C9 | |
| 237 | I-A12 | Pd(dppf)Cl₂•CH₂Cl₂, K₂CO₃, DMA, H₂O |
| | I-B37 | |
| | I-C9 | |
| 243 | I-A18 | Pd(dppf)Cl₂•CH₂Cl₂, K₂CO₃, DMA, H₂O |
| | 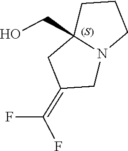 | |
| | I-B39 | |
| | I-C9 | |
| 262 | I-A12 | Pd(PPh₃)₂Cl₂, K₂CO₃, DMA, H₂O |
| | I-B1 | |
| | I-C29 | |
| 266 | I-A18 | Pd(PPh₃)₂Cl₂, K₂CO₃, DMA, H₂O |
| | I-B1 | |
| | I-C28 | |
| 267 | I-A12 | Pd(PPh₃)₂Cl₂, K₂CO₃, DMA, H₂O |
| | I-B1 | |
| | I-C29 | |
| 268 | I-A3 | Pd(PPh₃)₂Cl₂, K₂CO₃, DMA, H₂O |
| | I-B1 | |
| | I-C29 | |
| 281 | 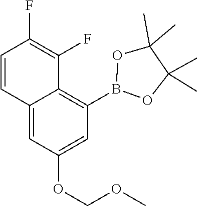 [SMILES: COCOc1cc(B2OC(C)(C)C(C)(C)O2)c2c(F)c(F)ccc2c1] | cataCXium A Pd-G₃, K₃PO₄, DMA, H₂O |
| | I-A37 | |
| | I-B10 | |
| | I-C29 | |
| 289 | I-A28 | Pd(dppf)Cl₂•CH₂Cl₂, K₂CO₃, DMA, H₂O |
| | I-B10 | |
| | I-C29 | |
| 290 | 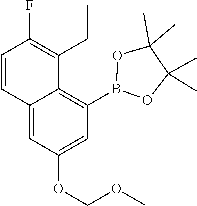 [SMILES: CCc1c(F)ccc2cc(OCOC)cc(B3OC(C)(C)C(C)(C)O3)c12] | cataCXium A Pd-G₃, K₃PO₄, DMA, H₂O |
| | I-A38 | |
| | I-B10 | |
| | I-C29 | |
| 291 | I-A40 | cataCXium A Pd-G₃, K₃PO₄, DMA, H₂O |
| | I-B10 | |
| | I-C29 | |
| 297 | I-A4 | RuPhos Pd G3, K₃PO₄, DMA, H₂O |
| | I-B1 | |
| | I-C29 | |
| 298 | 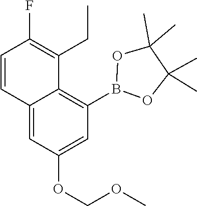 [SMILES: CCc1c(F)ccc2cc(OCOC)cc(B3OC(C)(C)C(C)(C)O3)c12] | cataCXium A Pd-G₃, K₃PO₄, DMA, H₂O |
| | I-A38 | |
| | I-B1 | |
| | I-C31 | |
| 300 | 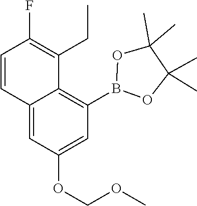 [SMILES: CCc1c(F)ccc2cc(OCOC)cc(B3OC(C)(C)C(C)(C)O3)c12] | RuPhos Pd G3, K₃PO₄, DMA, H₂O |
| | I-A38 | |
| | I-B1 | |
| | I-C32 | |
| 302 | I-A42 | RuPhos Pd G3, K₃PO₄, DMA, H₂O |
| | I-B1 | |
| | I-C29 | |
| 305 | I-A15 | RuPhos Pd G3, K₃PO₄, DMA, H₂O |
| | I-B1 | |
| | I-C29 | |
| 306 | I-A44 | RuPhos Pd G3, K₃PO₄, DMA, H₂O |
| | I-B1 | |
| | I-C29 | |
| 311 | I-A17 | RuPhos Pd G3, K₃PO₄, DMA, H₂O |
| | I-B1 | |
| | I-C29 | |
| 312 | I-A45 | RuPhos Pd G3, K₃PO₄, DMA, H₂O |
| | I-B1 | |
| | I-C29 | |
| 313 | 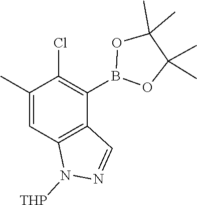 | RuPhos Pd G3, K₃PO₄, DMA, H₂O |
| | I-A46 | |
| | I-B1 | |
| | I-C29 | |
| 340 | I-A69 | RuPhos Pd G3, K₃PO₄, p-dioxane, |
| | I-B1 | H₂O |
| | I-C29 | |
| 361 | I-A71 | RuPhos Pd G3, K₃PO₄, p-dioxane, |
| | I-B1 | H₂O |
| | I-C29 | |
| 395 | I-A84 | RuPhos Pd G3, K₃PO₄, p-dioxane, |
| | I-B40 | H₂O |
| | I-C29 | |
| 396 | I-A84 | RuPhos Pd G3, K₃PO₄, p-dioxane, |
| | I-B2 | H₂O |
| | I-C29 | |
| 397 | I-A38 | RuPhos Pd G3, K₃PO₄, p-dioxane, |
| | I-B40 | H₂O |
| | I-C29 | |

Note:
intermediates I-A7, I-A30, I-A37, I-A38, I-A46, I-B2, I-B5, I-B6, I-B7, I-B9, I-B12, I-B17, I-B20, I-B24, I-B25, I-B28, I-B29, I-B30, I-B35, I-B37 and I-B39 were all commercially available.

### Compounds 20 and 21 3-(10-((R or S)-1-(2-aminopyridin-3-yl)ethyl)-2-(((S)-1-(2,2-difluoroethyl)azetidin-2-yl)methoxy)-4-fluoro-9,10-dihydro-8H-7-oxa-1,3,6,10-tetraazacyclohepta[de]naphthalen-5-yl)-5-chloro-4-cyclopropylphenol (diastereomer 1 and diastereomer 2)

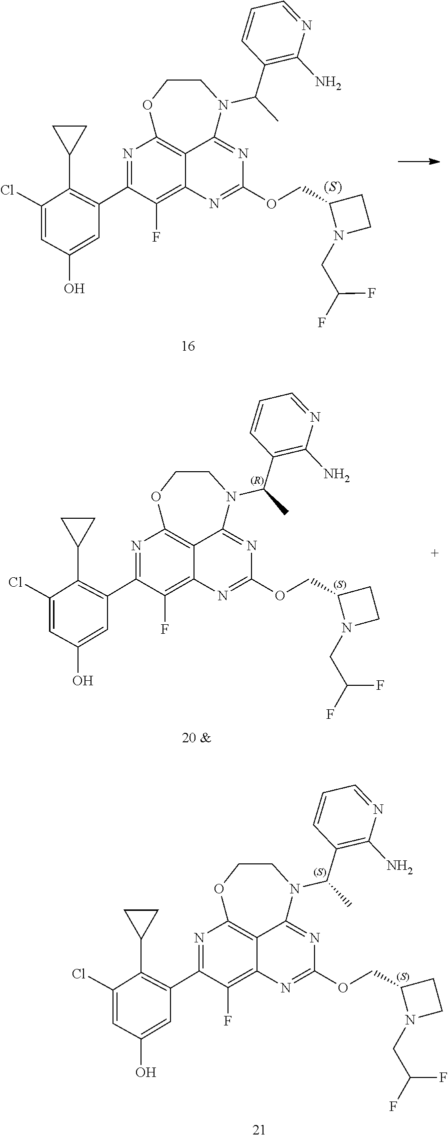

The compound 16 was resolved by chiral HPLC to obtain one pair of diastereomers. Chiral HPLC resolution conditions: column: IC (2×25 cm); mobile phase: ethanol (0.1% ammonia water); Flow rate: 15 mL/minute; detector: UV 254 nm.

First eluate (compound 20, diastereomer 1, chiral HPLC analysis conditions: column: IC (0.46×25 cm); mobile phase: ethanol (0.1% diethylamine), RT=4.775 minutes), de %=100%, [M+H]⁺ 642.3. ¹H NMR (400 MHz, CD₃OD) δ 7.99-7.93 (m, 1H), 7.81-7.72 (m, 1H), 6.92 (d, J=2.6 Hz, 1H), 6.82-6.76 (m, 1H), 6.73 (d, J=2.6 Hz, 1H), 6.66-6.58 (m, 1H), 6.04-5.70 (m, 1H), 4.58-4.28 (m, 4H), 3.83-3.70 (m, 2H), 3.64-3.45 (m, 2H), 3.26-3.09 (m, 2H), 2.92-2.75 (m, 1H), 2.23-2.11 (m, 2H), 1.91-1.79 (m, 1H), 1.72-1.61 (m, 3H), 0.77-0.59 (m, 2H), 0.27-0.05 (m, 2H).

Second eluate (compound 21, diastereomer 2, chiral HPLC analysis conditions: column: IC (0.46×25 cm); mobile phase: ethanol (0.1% diethylamine), RT=5.926 minutes), de %=96.36%, [M+H]⁺ 642.3. ¹H NMR (400 MHz, CD₃OD) δ 8.00-7.92 (m, 1H), 7.80-7.72 (m, 1H), 6.96-6.90 (m, 1H), 6.82-6.76 (m, 1H), 6.75-6.72 (m, 1H), 6.67-6.58 (m, 1H), 6.04-5.66 (m, 1H), 4.62-4.29 (m, 4H), 3.86-3.67 (m, 2H), 3.61-3.48 (m, 2H), 3.27-3.09 (m, 2H), 2.92-2.71 (m, 1H), 2.21-2.14 (m, 2H), 1.90-1.81 (m, 1H), 1.70-1.61 (m, 3H), 0.74-0.61 (m, 2H), 0.20-0.07 (m, 2H).

The compounds in the table below were prepared by following the chiral resolution conditions of compounds 20 and 21:

| | | | | | Preparation methods |
|---|---|---|---|---|---|
| | | | LC-MS | | and chiral HPLC |
| Compound | Structural formula | Name | [M + H]⁺ | ¹HNMR | resolution conditions |
| 23 | 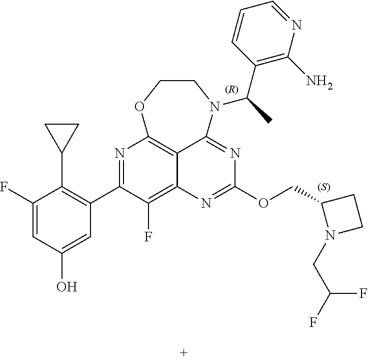 | 3-(10-((R or S)-1-(2- aminopyridin-3-yl)ethyl)-2-(((S)- 1-(2,2-difluoroethyl)azetidin-2- yl)methoxy)-4-fluoro-9,10- dihydro-8H-7-oxa-1,3,6,10- tetraazacyclohepta[de]naphthalen- 5-yl)-4-cyclopropyl-5- fluorophenol (diastereomer 1) | 626.3 | (400 MHz, CD₃OD) δ 7.97-7.91 (m, 1H), 7.78-7.72 (m, 1H), 6.80- 6.75 (m, 1H), 6.66-6.52 (m, 3H), 6.05-5.69 (m, 1H), 4.58-4.49 (m, 2H), 4.49-4.26 (m, 2H), 3.81-3.71 (m, 2H), 3.56-3.48 (m, 2H), 3.24- 3.08 (m, 2H), 2.89-2.73 (m, 1H), 2.21-2.12 (m, 2H), 1.75-1.68 (m, 1H), 1.68-1.62 (m, 3H), 0.64-0.52 (m, 2H), 0.32-0.17 (m, 2H). | It was obtained by resolving the compound prepared from I-A8, I-B4 and I-C3 by following the preparation steps of the compound 2; chiral HPLC resolution conditions: column: IC (2 × 25 cm); mobile phase: ethanol (0.1% ammonia water). |
| | 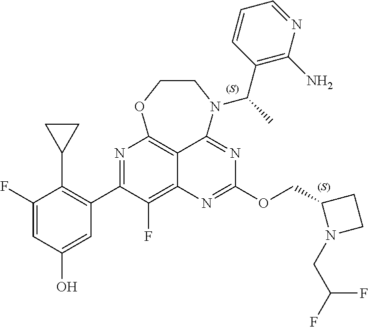 23 & 24 | | | | |
| 24 | | 3-(10-((R or S)-1-(2- | 626.3 | (400 MHz, CD₃OD) δ 7.98-7.91 | |
| | | aminopyridin-3-yl)ethyl)-2-(((S)- | | (m, 1H), 7.78-7.72 (m, 1H), 6.81- | |
| | | 1-(2,2-difluoroethyl)azetidin-2- | | 6.75 (m, 1H), 6.65-6.51 (m, 3H), | |
| | | yl)methoxy)-4-fluoro-9,10- | | 6.02-5.67 (m, 1H), 4.61-4.49 (m, | |
| | | dihydro-8H-7-oxa-1,3,6,10- | | 2H), 4.48-4.29 (m, 2H), 3.84-3.67 | |
| | | tetraazacyclohepta[de]naphthalen- | | (m, 2H), 3.56-3.46 (m, 2H), 3.23- | |
| | | 5-yl)-4-cyclopropyl-5- | | 3.09 (m, 2H), 2.88-2.73 (m, 1H), | |
| | | fluorophenol (diastereomer 2) | | 2.23-2.11 (m, 2H), 1.76-1.67 (m, | |
| | | | | 1H), 1.67-1.61 (m, 3H), 0.63-0.52 | |
| | | | | (m, 2H), 0.30-0.18 (m, 2H). | |
| 26 | 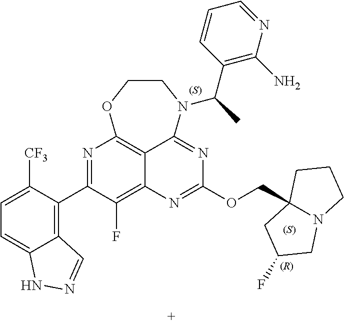 | 3-((1R or 1S)-1-(4-fluoro-2- (((2R,7aS)-2-fluorotetrahydro- 1H-pyrrolizin-7a(5H)- yl)methoxy)-5-(5- (trifluoromethyl)-1H-indazol-4- yl)-8,9-dihydro-10H-7-oxa- 1,3,6,10- tetraazacyclohepta[de]naphthalen- 10-yl)ethyl)pyridin-2-amine (diastereomer 1) | 668.3 | (400 MHz, CD₃OD) δ 7.96 (d, J = 4.3 Hz, 1H), 7.86-7.74 (m, 4H), 6.78 (dd, J = 7.4, 5.2 Hz, 1H), 6.67-6.57 (m, 1H), 5.41-5.21 (m, 1H), 4.51-4.27 (m, 4H), 3.86-3.74 (m, 1H), 3.64-3.52 (m, 1H), 3.28- 3.16 (m, 3H), 3.07-2.96 (m, 1H), 2.42-2.12 (m, 3H), 2.04-1.88 (m, 3H), 1.69-1.64 (m, 3H). | It was obtained by resolving the compound 15; chiral HPLC resolution conditions: column: IC (2 × 25 cm); mobile phase: ethanol (0.1% ammonia water). |
| | 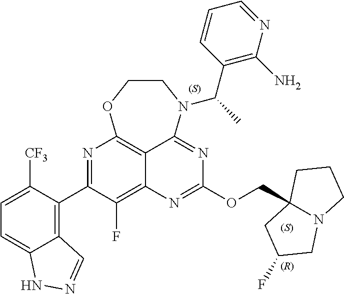 26 & 27 | | | | |
| 27 | | 3-((1R or 1S)-1-(4-fluoro-2- | 668.3 | (400 MHz, CD₃OD) δ 7.96 (d, J = | |
| | | (((2R,7aS)-2-fluorotetrahydro- | | 4.3 Hz, 1H), 7.86-7.74 (m, 4H), | |
| | | 1H-pyrrolizin-7a(5H)- | | 6.78 (dd, J = 7.4, 5.2 Hz, 1H), | |
| | | yl)methoxy)-5-(5- | | 6.67-6.57 (m, 1H), 5.41-5.21 (m, | |
| | | (trifluoromethyl)-1H-indazol-4- | | 1H), 4.51-4.27 (m, 4H), 3.86-3.74 | |
| | | yl)-8,9-dihydro-10H-7-oxa- | | (m, 1H), 3.64-3.52 (m, 1H), 3.28- | |
| | | 1,3,6,10- | | 3.16 (m, 3H), 3.07-2.96 (m, 1H), | |
| | | tetraazacyclohepta[de]naphthalen- | | 2.42-2.12 (m, 3H), 2.04-1.88 (m, | |
| | | 10-yl)ethyl)pyridin-2-amine | | 3H), 1.69-1.64 (m, 3H). | |
| | | (diastereomer 2) | | | |
| 29 | 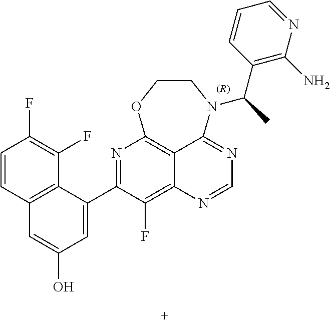 | (R or S)-4-(10-(1-(2- aminopyridin-3-yl)ethyl)-4- fluoro-9,10-dihydro-8H-7-oxa- 1,3,6,10- tetraazacyclohepta[de]naphthalen- 5-yl)-5,6-difluoronaphthalen-2- ol (enantiomer 1) | 505.2 | (400 MHz, CD₃OD) δ 8.71 (s, 1H), 7.96-7.92 (m, 1H), 7.76 (d, J = 7.6 Hz, 1H), 7.60-7.54 (m, 1H), 7.40-7.34 (m, 1H), 7.31-7.27 (m, 1H), 7.23-7.19 (m, 1H), 6.86-6.71 (m, 2H), 4.57-4.26 (m, 2H), 3.82- 3.70 (m, 1H), 3.63-3.55 (m, 1H), 1.63 (d, J = 6.8 Hz, 3H). | It was obtained by resolving the compound 22; chiral HPLC resolution conditions: column: IC (2 × 25 cm); mobile phase: n- heptane/ethanol (0.1% ammonia water) = 50: 50. |
| | 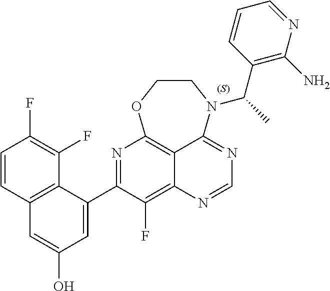 29 & 30 | | | | |
| 30 | | (R or S)-4-(10-(1-(2- | 505.2 | (400 MHz, CD₃OD) δ 8.71 (s, | |
| | | aminopyridin-3-yl)ethyl)-4- | | 1H), 7.96-7.92 (m, 1H), 7.76 (d, J = | |
| | | fluoro-9,10-dihydro-8H-7-oxa- | | 7.4 Hz, 1H), 7.60-7.52 (m, 1H), | |
| | | 1,3,6,10- | | 7.40-7.32 (m, 1H), 7.27 (s, 1H), | |
| | | tetraazacyclohepta[de]naphthalen- | | 7.22-7.18 (m, 1H), 6.85-6.71 (m, | |
| | | 5-yl)-5,6-difluoronaphthalen-2- | | 2H), 4.55-4.24 (m, 2H), 3.84-3.72 | |
| | | ol (enantiomer 2) | | (m, 1H), 3.63-3.55 (m, 1H), 1.63 | |
| | | | | (d, J = 6.8 Hz, 3H). | |
| 31 | 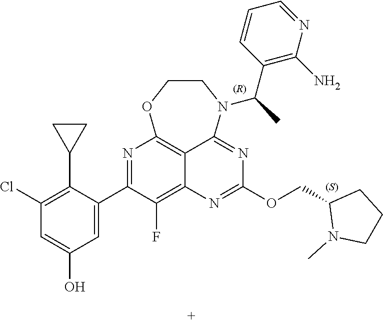 | 3-(10-((R or S)-1-(2- aminopyridin-3-yl)ethyl)-4- fluoro-2-(((S)-1-methylpyrrolidin- 2-yl)methoxy)-9,10-dihydro-8H- 7-oxa-1,3,6,10- tetraazacyclohepta[de]naphthalen- 5-yl)-5-chloro-4- cyclopropylphenol (diastereomer 1) | 606.2 | (400 MHz, CD₃OD) δ 7.97-7.91 (m, 1H), 7.78-7.70 (m, 1H), 6.87 (d, J = 2.6 Hz, 1H), 6.80-6.73 (m, 1H), 6.69 (d, J = 2.6 Hz, 1H), 6.65-6.58 (m, 1H), 4.58-4.25 (m, 4H), 3.80-3.48 (m, 2H), 3.16-3.02 (m, 1H), 2.90-2.73 (m, 1H), 2.50 (s, 3H), 2.42-2.29 (m, 1H), 2.19- 2.04 (m, 1H), 1.90-1.76 (m, 4H), 1.68-1.60 (m, 3H), 0.71-0.58 (m, 2H), 0.20-0.00 (m, 2H). | It was obtained by resolving the compound prepared from I-A3, I-B3 and I-C3 by following the preparation steps of the compound 2; chiral HPLC resolution conditions: column: IC (2 × 25 cm); mobile phase: ethanol (0.1% ammonia water). |
| | 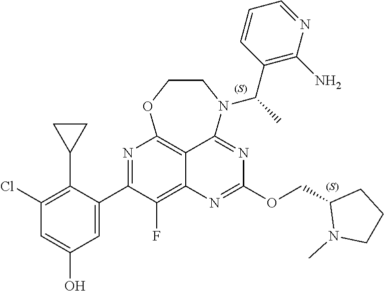 31 & 32 | | | | |
| 32 | | 3-(10-((R or S)-1-(2- | 606.3 | (400 MHz, CD₃OD) δ 7.98-7.91 | |
| | | aminopyridin-3-yl)ethyl)-4- | | (m, 1H), 7.78-7.71 (m, 1H), 6.80 | |
| | | fluoro-2-(((S)-1-methylpyrrolidin- | | (d, J = 2.6 Hz, 1H), 6.79-6.74 (m, | |
| | | 2-yl)methoxy)-9,10-dihydro-8H- | | 1H), 6.65-6.56 (m, 2H), 4.54-4.27 | |
| | | 7-oxa-1,3,6,10- | | (m, 4H), 3.79-3.44 (m, 2H), 3.14- | |
| | | tetraazacyclohepta[de]naphthalen- | | 3.05 (m, 1H), 2.88-2.74 (m, 1H), | |
| | | 5-yl)-5-chloro-4- | | 2.51 (s, 3H), 2.42-2.29 (m, 1H), | |
| | | cyclopropylphenol (diastereomer 2) | | 2.21-2.04 (m, 1H), 1.86-1.74 (m, | |
| | | | | 4H), 1.68-1.60 (m, 3H), 0.69-0.52 | |
| | | | | (m, 2H), 0.20-0.04 (m, 2H). | |
| 34 | 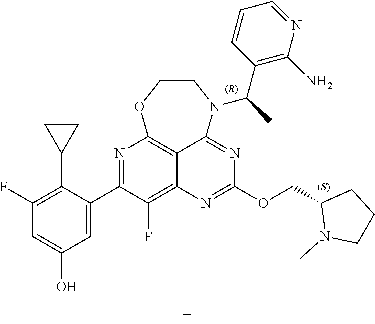 | 3-(10-((R or S)-1-(2- aminopyridin-3-yl)ethyl)-4- fluoro-2-(((S)-1-methylpyrrolidin- 2-yl)methoxy)-9,10-dihydro-8H- 7-oxa-1,3,6,10- tetraazacyclohepta[de]naphthalen- 5-yl)-4-cyclopropyl-5- fluorophenol (diastereomer 1) | 590.3 | (400 MHz, CD₃OD) δ 7.97-7.91 (m, 1H), 7.77-7.72 (m, 1H), 6.80- 6.74 (m, 1H), 6.66-6.51 (m, 3H), 4.59-4.53 (m, 1H), 4.52-4.40 (m, 2H), 4.36-4.26 (m, 1H), 3.80-3.68 (m, 1H), 3.58-3.47 (m, 1H), 3.15- 3.05 (m, 1H), 2.90-2.79 (m, 1H), 2.53 (s, 3H), 2.44-2.34 (m, 1H), 2.18-2.06 (m, 1H), 1.89-1.68 (m, 4H), 1.67-1.60 (m, 3H), 0.63-0.51 (m, 2H), 0.31-0.17 (m, 2H). | It was obtained by resolving the compound prepared from I-A8, I-B3 and I-C3 by following the preparation steps of the compound 2; chiral HPLC resolution conditions: column: IC (2 × 25 cm); mobile phase: n- heptane/ethanol (0.1% ammonia water) = 50:50. |
| | 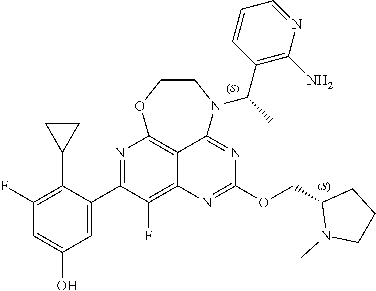 34 & 35 | | | | |
| 35 | | 3-(10-((R or S)-1-(2- | 590.3 | (400 MHz, CD₃OD) δ 7.97-7.91 | |
| | | aminopyridin-3-yl)ethyl)-4- | | (m, 1H), 7.78-7.72 (m, 1H), 6.80- | |
| | | fluoro-2-(((S)-1-methylpyrrolidin- | | 6.74 (m, 1H), 6.65-6.52 (m, 3H), | |
| | | 2-yl)methoxy)-9,10-dihydro-8H- | | 4.56-4.49 (m, 2H), 4.49-4.40 (m, | |
| | | 7-oxa-1,3,6,10- | | 1H), 4.36-4.27 (m, 1H), 3.79-3.68 | |
| | | tetraazacyclohepta[de]naphthalen- | | (m, 1H), 3.57-3.47 (m, 1H), 3.14- | |
| | | 5-yl)-4-cyclopropyl-5- | | 3.06 (m, 1H), 2.90-2.80 (m, 1H), | |
| | | fluorophenol (diastereomer 2) | | 2.53 (s, 3H), 2.44-2.34 (m, 1H), | |
| | | | | 2.18-2.07 (m, 1H), 1.89-1.68 (m, | |
| | | | | 4H), 1.68-1.61 (m, 3H), 0.64-0.52 | |
| | | | | (m, 2H), 0.30-0.19 (m, 2H). | |
| 38 | 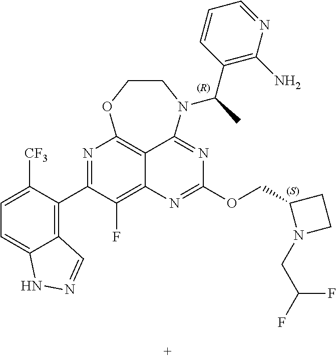 | 3-((1R or 1S)-1-(2-(((S)-1-(2,2- difluoroethyl)azetidin-2- yl)methoxy)-4-fluoro-5-(5- (trifluoromethyl)-1H-indazol-4- yl)-8,9-dihydro-10H- 7-oxa-1,3,6,10- tetraazacyclohepta[de]naphthalen- 10-yl)ethyl)pyridin-2-amine (diastereomer 1) | 660.3 | (400 MHz, CD₃OD) δ 7.98-7.93 (m, 1H), 7.83-7.75 (m, 4H), 6.82- 6.73 (m, 1H), 6.68-6.56 (m, 1H), 6.01-5.68 (m, 1H), 4.62-4.31 (m, 4H), 3.82-3.73 (m, 2H), 3.62-3.49 (m, 2H), 3.25-3.11 (m, 2H), 2.88- 2.75 (m, 1H), 2.21-2.13 (m, 2H), 1.69-1.64 (m, 3H). | It was obtained by resolving the compound 25; chiral HPLC resolution conditions: column: IC (2 × 25 cm); mobile phase: ethanol (0.1% ammonia water). |
| | 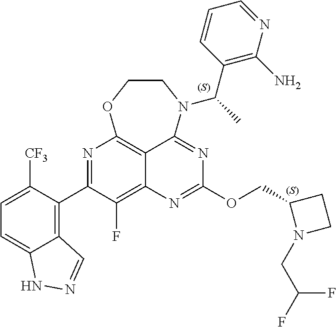 38 & 39 | | | | |
| 39 | | 3-((1R or 1S)-1-(2-(((S)-1-(2,2- | 660.3 | (400 MHz, CD₃OD) δ 7.98-7.93 | |
| | | difluoroethyl)azetidin-2- | | (m, 1H), 7.83-7.75 (m, 4H), 6.82- | |
| | | yl)methoxy)-4-fluoro-5-(5- | | 6.73 (m, 1H), 6.68-6.56 (m, 1H), | |
| | | (trifluoromethyl)-1H-indazol-4- | | 6.01-5.68 (m, 1H), 4.62-4.31 (m, | |
| | | yl)-8,9-dihydro-10H-7-oxa- | | 4H), 3.82-3.73 (m, 2H), 3.62-3.49 | |
| | | 1,3,6,10- | | (m, 2H), 3.25-3.11 (m, 2H), 2.88- | |
| | | tetraazacyclohepta[de]naphthalen- | | 2.75 (m, 1H), 2.21-2.13 (m, 2H), | |
| | | 10-yl)ethyl)pyridin-2-amine | | 1.69-1.64 (m, 3H). | |
| | | (diastereomer 2) | | | |
| 41 | 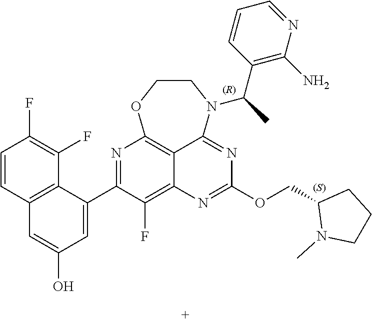 | 4-(10-((R or S)-1-(2- aminopyridin-3-yl)ethyl)-4- fluoro-2-(((S)-1-methylpyrrolidin- 2-yl)methoxy)-9,10-dihydro-8H- 7-oxa-1,3,6,10- tetraazacyclohepta[de]naphthalen- 5-yl)-5,6-difluoronaphthalen-2- ol (diastereomer 1) | 618.3 | (400 MHz, CD₃OD) δ 7.97-7.93 (m, 1H), 7.77-7.73 (m, 1H), 7.60- 7.55 (m, 1H), 7.41-7.33 (m, 1H), 7.30-7.26 (m, 1H), 7.21-7.16 (m, 1H), 6.80-6.75 (m, 1H), 6.67-6.58 (m, 1H), 4.60-4.42 (m, 3H), 4.40- 4.29 (m, 1H), 3.83-3.70 (m, 1H), 3.60-3.50 (m, 1H), 3.18-3.09 (m, 1H), 2.96-2.85 (m, 1H), 2.56 (s, 3H), 2.49-2.38 (m, 1H), 2.19-2.09 (m, 1H), 1.90-1.75 (m, 3H), 1.69- 1.61 (m, 3H). | It was obtained by resolving the compound 6; chiral HPLC resolution conditions: column: IC (2 × 25 cm); mobile phase: n- heptane/ethanol (0.1% ammonia water) = 50:50. |
| | 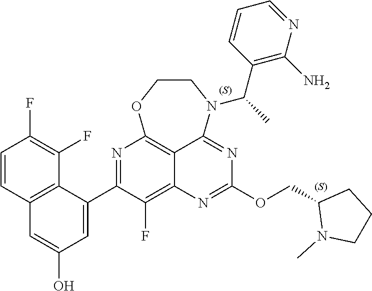 41 & 42 | | | | |
| 42 | | 4-(10-((R or S)-1-(2- | 618.3 | (400 MHz, CD₃OD) δ 7.97-7.93 | |
| | | aminopyridin-3-yl)ethyl)-4- | | (m, 1H), 7.78-7.73 (m, 1H), 7.60- | |
| | | fluoro-2-(((S)-1-methylpyrrolidin- | | 7.55 (m, 1H), 7.41-7.33 (m, 1H), | |
| | | 2-yl)methoxy)-9,10-dihydro-8H- | | 7.30-7.26 (m, 1H), 7.23-7.16 (m, | |
| | | 7-oxa-1,3,6,10- | | 1H), 6.80-6.75 (m, 1H), 6.66-6.57 | |
| | | tetraazacyclohepta[de]naphthalen- | | (m, 1H), 4.61-4.41 (m, 3H), 4.40- | |
| | | 5-yl)-5,6-difluoronaphthalen-2- | | 4.29 (m, 1H), 3.80-3.71 (m, 1H), | |
| | | ol (diastereomer 2) | | 3.61-3.49 (m, 1H), 3.20-3.12 (m, | |
| | | | | 1H), 3.00-2.90 (m, 1H), 2.59 (s, | |
| | | | | 3H), 2.52-2.42 (m, 1H), 2.20-2.10 | |
| | | | | (m, 1H), 1.91-1.76 (m, 3H), 1.69- | |
| | | | | 1.61 (m, 3H). | |
| 43 | 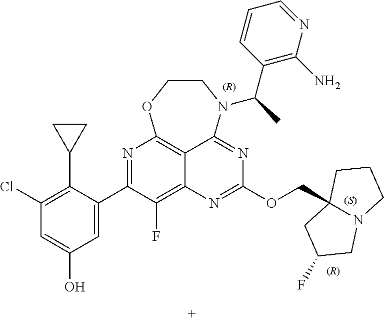 | 3-(10-((R or S)-1-(2- aminopyridin-3-yl)ethyl)-4- fluoro-2-(((2R,7aS)-2- fluorotetrahydro-1H-pyrrolizin- 7a(5H)-yl)methoxy)-9,10- dihydro-8H-7-oxa-1,3,6,10- tetraazacyclohepta[de]naphthalen- 5-yl)-5-chloro-4- cyclopropylphenol (diastereomer 1) | 650.2 | (400 MHz, CD₃OD) δ 7.99-7.92 (m, 1H), 7.78-7.72 (m, 1H), 6.91 (d, J = 2.6 Hz, 1H) 6.80-6.75 (m, 1H), 6.64-6.55 (m, 1H), 6.65-6.55 (m, 1H), 5.41-5.20 (m, 1H), 4.50- 4.22 (m, 4H), 3.82-3.50 (m, 2H), 3.26-3.15 (m, 2H), 3.07-2.95 (m, 1H), 2.44-2.10 (m, 3H), 2.05-1.81 (m, 4H), 1.71-1.53 (m, 4H), 0.78- 0.57 (m, 2H), 0.21-0.04 (m, 2H). | It was obtained by resolving the compound 4; chiral HPLC resolution conditions: column: IC (2 × 25 cm); mobile phase: ethanol (0.1% ammonia water). |
| | 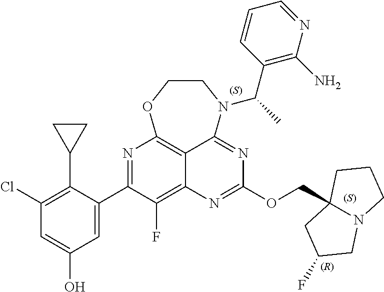 43 & 44 | | | | |
| 44 | | 3-(10-((R or S)-1-(2- | 650.3 | (400 MHz, CD₃OD) δ 8.00-7.91 | |
| | | aminopyridin-3-yl)ethyl)-4- | | (m, 1H), 7.79-7.71 (m, 1H), 6.94- | |
| | | fluoro-2-(((2R,7aS)-2- | | 6.89 (m, 1H), 6.80-6.74 (m, 1H), | |
| | | fluorotetrahydro-1H-pyrrolizin- | | 6.73-6.70 (m, 1H), 6.65-6.56 (m, | |
| | | 7a(5H)-yl)methoxy)-9,10- | | 1H), 5.43-5.18 (m, 1H), 4.50-4.26 | |
| | | dihydro-8H-7-oxa-1,3,6,10- | | (m, 4H), 3.83-3.69 (m, 1H), 3.59- | |
| | | tetraazacyclohepta[de]naphthalen- | | 3.45 (m, 1H), 3.25-3.15 (m, 2H), | |
| | | 5-yl)-5-chloro-4- | | 3.07-2.97 (m, 1H), 2.38-2.13 (m, | |
| | | cyclopropylphenol (diastereomer | | 3H), 2.01-1.84 (m, 4H), 1.69-1.54 | |
| | | 2) | | (m, 4H), 0.74-0.59 (m, 2H), 0.20- | |
| | | | | 0.03 (m, 2H). | |
| 48 | 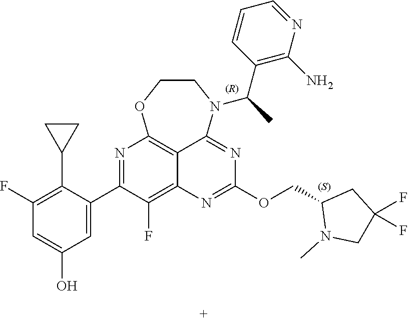 | 3-(10-((R or S)-1-(2- aminopyridin-3-yl)ethyl)-2-(((S)- 4,4-difluoro-1-methylpyrrolidin- 2-yl)methoxy)-4-fluoro-9,10- dihydro-8H-7-oxa-1,3,6,10- tetraazacyclohepta[de]naphthalen- 5-yl)-4-cyclopropyl-5- fluorophenol (diastereomer 1) | 626.3 | (400 MHz, CD₃OD) δ 7.97-7.91 (m, 1H), 7.78-7.72 (m, 1H), 6.80- 6.75 (m, 1H), 6.66-6.52 (m, 3H), 4.63-4.52 (m, 2H), 4.49-4.41 (m, 1H), 4.38-4.26 (m, 1H), 3.81-3.69 (m, 1H), 3.57-3.49 (m, 1H), 3.42- 3.34 (m, 1H), 3.12-3.02 (m, 1H), 2.80-2.67 (m, 1H), 2.61-2.50 (m, 1H), 2.49 (s, 3H), 2.39-3.24 (m, 1H), 1.75-1.67 (m, 1H), 1.67-1.60 (m, 3H), 0.64-0.51 (m, 2H), 0.30- 0.18 (m, 2H). | It was obtained by resolving the compound 45; chiral HPLC resolution conditions: column: IC (2 × 25 cm); mobile phase: n- heptane/ethanol (0.1% ammonia water) = 70:30. |
| | 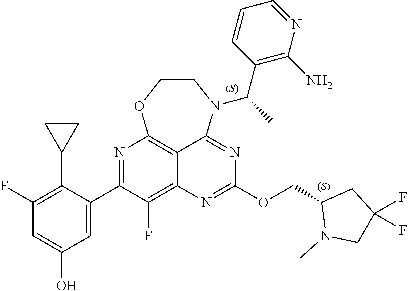 48 & 49 | | | | |
| 49 | | 3-(10-((R or S)-1-(2- | 626.3 | (400 MHz, CD₃OD) δ 7.98-7.91 | |
| | | aminopyridin-3-yl)ethyl)-2-(((S)- | | (m, 1H), 7.78-7.73 (m, 1H), 6.80- | |
| | | 4,4-difluoro-1-methylpyrrolidin- | | 6.75 (m, 1H), 6.65-6.51 (m, 3H), | |
| | | 2-yl)methoxy)-4-fluoro-9,10- | | 4.65-4.53 (m, 2H), 4.48-4.41 (m, | |
| | | dihydro-8H-7-oxa-1,3,6,10- | | 1H), 4.36-4.28 (m, 1H), 3.78-3.69 | |
| | | tetraacyclohepta[de]naphthalen- | | (m, 1H), 3.56-3.48 (m, 1H), 3.42- | |
| | | 5-yl)-4-cyclopropyl-5- | | 3.34 (m, 1H), 3.11-3.03 (m, 1H), | |
| | | fluorophenol (diastereomer 2) | | 2.80-2.66 (m, 1H), 2.62-2.50 (m, | |
| | | | | 1H), 2.48 (s, 3H), 2.39-2.24 (m, | |
| | | | | 1H), 1.75-1.67 (m, 1H), 1.67-1.60 | |
| | | | | (m, 3H), 0.63-0.52 (m, 2H), 0.30- | |
| | | | | 0.19 (m, 2H). | |
| 50 | 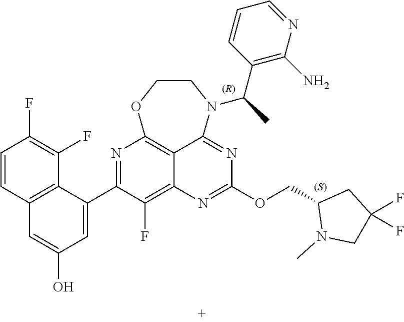 | 4-(10-((R or S)-1-(2- aminopyridin-3-yl)ethyl)-2-(((S)- 4,4-difluoro-1-methylpyrrolidin- 2-yl)methoxy)-4-fluoro-9,10- dihydro-8H-7-oxa-1,3,6,10- tetraazacyclohepta[de]naphthalen- 5-yl)-5,6-difluoronaphthalen-2- ol (diasteromer 1) | 654.3 | (400 MHz, CD₃OD/CDCl₃ = 1/1) δ 7.98-7.91 (m, 1H), 7.69-7.64 (m, 1H), 7.50-7.45 (m, 1H), 7.32-7.15 (m, 3H), 6.77-6.72 (m, 1H), 6.67- 6.60 (m, 1H), 4.62-4.21 (m, 4H), 3.73-3.64 (m, 1H), 3.57-3.48 (m, 1H), 3.42-3.34 (m, 1H), 3.10-3.01 (m, 1H), 2.77-2.65 (m, 1H), 2.60- 2.49 (m, 1H), 2.48 (s, 3H), 2.37- 2.22 (m, 1H), 1.67-1.58 (m, 3H). | It was obtained by resolving the compound 46; chiral HPLC resolution conditions: column: IC (2 × 25 cm); mobile phase: n-heptane/ ethanol (0.1% ammonia water) = 70:30. |
| | 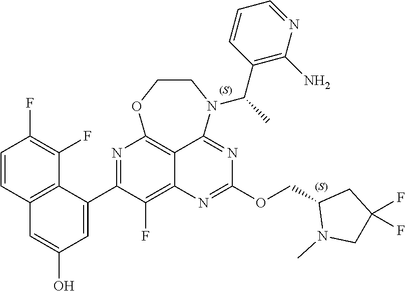 50 & 51 | | | | |
| 51 | | 4-(10-((R or S)-1-(2- | 654.3 | (400 MHz, CD₃OD/CDCl₃ = 1/1) δ | |
| | | aminopyridin-3-yl)ethyl)-2-(((S)- | | 7.97-7.91 (m, 1H), 7.68-7.63 (m, | |
| | | 4,4-difluoro-1-methylpyrrolidin- | | 1H), 7.49-7.44 (m, 1H), 7.31-7.15 | |
| | | 2-yl)methoxy)-4-fluoro-9,10- | | (m, 3H), 6.77-6.72 (m, 1H), 6.67- | |
| | | dihydro-8H-7-oxa-1,3,6,10- | | 6.59 (m, 1H), 4.59-4.21 (m, 4H), | |
| | | tetraazacyclohepta[de]naphthalen- | | 3.72-3.63 (m, 1H), 3.56-3.47 (m, | |
| | | 5-yl)-5,6-difluoronaphthalen-2- | | 1H), 3.41-3.33 (m, 1H), 3.10-3.01 | |
| | | ol (diastereomer 2) | | (m, 1H), 2.76-2.64 (m, 1H), 2.60- | |
| | | | | 2.49 (m, 1H), 2.47 (s, 3H), 2.37- | |
| | | | | 2.24 (m, 1H), 1.66-1.58 (m, 3H). | |
| 53 | 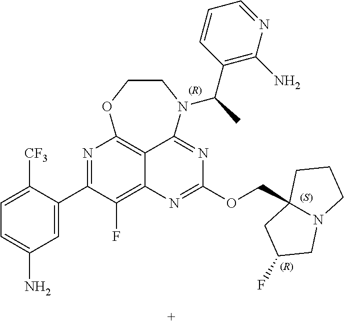 | 3-((R or S)-1-(5-(5-amino-2- (trifluoromethyl)phenyl)-4- fluoro-2-(((2R,7aS)-2- fluorotetrahydro-1H-pyrrolizin- 7a(5H)-yl)methoxy)-8,9- dihydro-10H-7-oxa-1,3,6,10- tetraazacyclohepta[de]naphthalen- 10-yl)ethyl)pyridin-2-amine (diastereomer 1) | 643.2 | (400 MHz, CD₃OD) δ 8.22 (d, J = 7.6 Hz, 1H), 7.96 (dd, J = 6.3, 1.3 Hz, 1H), 7.57 (d, J = 8.7 Hz, 1H), 7.09-7.02 (m, 1H), 6.98 (dd, J = 8.6, 1.6 Hz, 1H), 6.81 (d, J = 2.2 Hz, 1H), 6.59 (q, J = 6.8 Hz, 1H), 5.65-5.52 (m, 1H), 4.80-4.70 (m, 2H), 4.62-4.45 (m, 2H), 4.06-3.81 (m, 4H), 3.75-3.56 (m, 1H), 3.45- 3.41 (m, 1H), 2.83-2.52 (m, 2H), 2.50-2.39 (m, 1H), 2.33-2.30 (m, 2H), 2.21 (s, 1H), 1.72 (d, J = 6.9 Hz, 3H). | It was obtained by resolving the compound 18; chiral HPLC resolution conditions: column: OJH (0.46 × 15 cm); mobile phase: carbon dioxide/ ethanol (0.05% diethylamine) = 60:40. |
| | 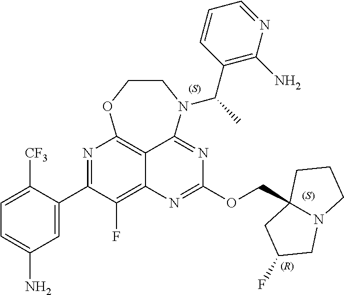 53 & 54 | | | | |
| 54 | | 3-((R or S)-1-(5-(5-amino-2- | 643.3 | (400 MHz, CD₃OD) δ 8.22 (d, J = | |
| | | (trifluoromethyl)phenyl)-4- | | 7.6 Hz, 1H), 7.96 (dd, J = 6.3, 1.3 | |
| | | fluoro-2-(((2R,7aS)-2- | | Hz, 1H), 7.57 (d, J = 8.7 Hz, 1H), | |
| | | fluorotetrahydro-1H-pyrrolizin- | | 7.09-7.02 (m, 1H), 6.98 (dd, J = | |
| | | 7a(5H)-yl)methoxy)-8,9-dihydro- | | 8.6, 1.6 Hz, 1H), 6.81 (d, J = 2.2 | |
| | | 10H-7-oxa-1,3,6,10- | | Hz, 1H), 6.59 (q, J = 6.8 Hz, 1H), | |
| | | tetraazacyclohepta[de]naphthalen- | | 5.65-5.52 (m, 1H), 4.80-4.70 (m, | |
| | | 10-yl)ethyl)pyridin-2-amine | | 2H), 4.62-4.45 (m, 2H), 4.06-3.81 | |
| | | (diastereomer 2) | | (m, 4H), 3.75-3.56 (m, 1H), 3.45- | |
| | | | | 3.41 (m, 1H), 2.83-2.52 (m, 2H), | |
| | | | | 2.50-2.39 (m, 1H), 2.33-2.30 (m, | |
| | | | | 2H), 2.21 (s, 1H), 1.72 (d, J = 6.9 | |
| | | | | Hz, 3H). | |
| 55 | 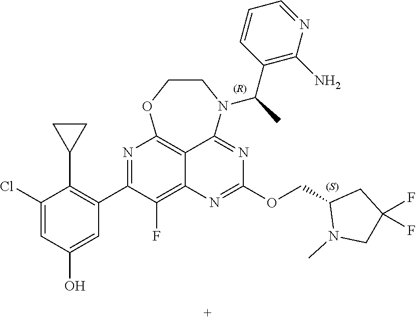 | 3-(10-((R or S)-1-(2- aminopyridin-3-yl)ethyl)-2-(((S)- 4,4-difluoro-1-methylpyrrolidin- 2-yl)methoxy)-4-fluoro-9,10- dihydro-8H-7-oxa-1,3,6,10- tetraazacyclohepta[de]naphthalen- 5-yl)-5-chloro-4- cyclopropylphenol (diastereomer 1) | 642.2 | (400 MHz, CD₃OD) δ 7.99-7.90 (m, 1H), 7.80-7.70 (m, 1H), 6.90 (d, J = 2.6 Hz, 1H), 6.81-6.74 (m, 1H), 6.71 (d, J = 2.6 Hz, 1H), 6.65-6.55 (m, 1H), 4.69-4.24 (m, 4H), 3.83-3.69 (m, 1H), 3.65-3.50 (m, 1H), 3.43-3.34 (m, 1H), 3.16- 3.02 (m, 1H), 2.83-2.66 (m, 1H), 2.60-2.45 (m, 4H), 2.41-2.25 (m, 1H), 1.92-1.77 (m, 1H), 1.70-1.59 (m, 3H), 0.73-0.58 (m, 2H), 0.22- 0.00 (m, 2H). | It was obtained by resolving the compound prepared from I-A3, I-B8 and I-C3 by following the preparation steps of the compound 2; chiral HPLC resolution conditions: column: IC (2 × 25 cm); mobile phase: ethanol (0.1% ammonia water). |
| | 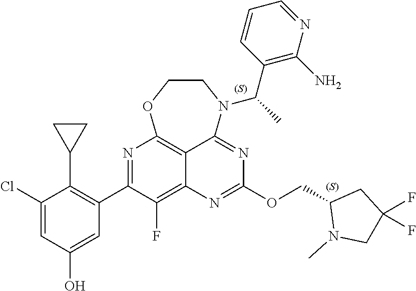 55 & 56 | | | | |
| 56 | | 3-(10-((R or S)-1-(2- | 642.2 | (400 MHz, CD₃OD) δ 7.98-7.91 | |
| | | aminopyridin-3-yl)ethyl)-2-(((S)- | | (m, 1H), 7.78-7.71 (m, 1H), 6.90 | |
| | | 4,4-difluoro-1-methylpyrrolidin- | | (d, J = 2.6 Hz, 1H), 6.81-6.74 (m, | |
| | | 2-yl)methoxy)-4-fluoro-9,10- | | 1H), 6.72 (d, J = 2.6 Hz, 1H), | |
| | | dihydro-8H-7-oxa-1,3,6,10- | | 6.65-6.55 (m, 1H), 4.66-4.40 (m, | |
| | | tetraazacyclohepta[de]naphthalen- | | 3H), 4.38-4.21 (m, 1H), 3.80-3.67 | |
| | | 5-yl)-5-chloro-4- | | (m, 1H), 3.61-3.45 (m, 1H), 3.44- | |
| | | cyclopropylphenol (diastereomer | | 3.33 (m, 1H), 3.14-3.03 (m, 1H), | |
| | | 2) | | 2.80-2.68 (m, 1H), 2.61-2.45 (m, | |
| | | | | 4H), 2.40-2.25 (m, 1H), 1.88-1.79 | |
| | | | | (m, 1H), 1.67-1.60 (m, 3H), 0.73- | |
| | | | | 0.58 (m, 2H), 0.18-0.02 (m, 2H). | |
| 57 | 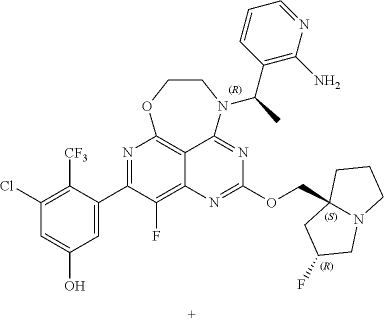 | 3-(10-((R or S)-1-(2- aminopyridin-3-yl)ethyl)-4- fluoro-2-(((2R,7aS)-2- fluorotetrahydro-1H-pyrrolizin- 7a(5H)-yl)methoxy)-9,10- dihydro-8H-7-oxa-1,3,6,10- tetraazacyclohepta[de]naphthalen- 5-yl)-5-chloro-4- (trifluoromethyl)phenol (diastereomer 1) | 678.2 | (400 MHz, CD₃OD) δ 7.98-7.91 (m, 1H), 7.78-7.70 (m, 1H), 7.12- 7.06 (m, 1H), 6.81-6.75 (m, 1H), 6.73-6.68 (m, 1H), 6.61-6.52 (m, 1H), 5.51-5.26 (m, 1H), 4.53-4.30 (m, 4H), 3.78-3.69 (m, 1H), 3.59- 3.51 (m, 1H), 3.42-3.37 (m, 1H), 3.18-3.08 (m, 1H), 2.48-2.15 (m, 4H), 2.12-1.91 (m, 4H), 1.70-1.61 (m, 3H). | It was obtained by resolving the compound prepared from I-A11, I-B1 and I-C3 by following the preparation steps of the compound 2; chiral HPLC resolution conditions: column: IC (2 × 25 cm); mobile phase: n-heptane/ ethanol (0.1% ammonia water) = 70:30. |
| | 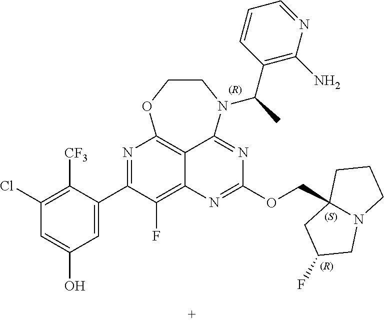 57 & 58 | | | | |
| 58 | | 3-(10-((R or S)-1-(2- | 678.2 | (400 MHz, CD₃OD) δ 7.98-7.91 | |
| | | aminopyridin-3-yl)ethyl)-4- | | (m, 1H), 7.77-7.71 (m, 1H), 7.09- | |
| | | fluoro-2-(((2R,7aS)-2- | | 7.04 (m, 1H), 6.79-6.74 (m, 1H), | |
| | | fluorotetrahydro-1H-pyrrolizin- | | 6.71-6.65 (m, 1H), 6.63-6.53 (m, | |
| | | 7a(5H)-yl)methoxy)-9,10- | | 1H), 5.43-5.24 (m, 1H), 4.49-4.29 | |
| | | dihydro-8H-7-oxa-1,3,6,10- | | (m, 4H), 3.83-3.69 (m, 1H), 3.64- | |
| | | tetraazacyclohepta[de]naphthalen- | | 3.49 (m, 1H), 3.38-3.31 (m, 1H), | |
| | | 5-yl)-5-chloro-4- | | 3.10-2.98 (m, 1H), 2.43-2.15 (m, | |
| | | (trifluoromethyl)phenol | | 4H), 2.08-1.86 (m, 4H), 1.70-1.60 | |
| | | (diastereomer 2) | | (m, 3H). | |
| 61 | 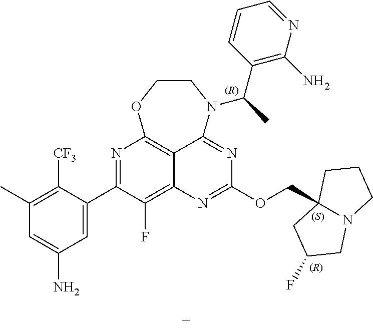 | 3-((R or S)-1-(5-(5-amino-3- methyl-2-(trifluoromethyl)phenyl)-4- fluoro-2-(((2R,7aS)-2- fluorotetrahydro-1H-pyrrolizin- 7a(5H)-yl)methoxy)-8,9- dihydro-10H-7-oxa-1,3,6,10- tetraazacyclohepta[de]naphthalen- 10-yl)ethyl)pyridin-2-amine (diastereomer 1) | 657.2 | (400 MHz, CD₃OD) δ 7.99-7.91 (m, 1H), 7.78-7.69 (m, 1H), 6.81- 6.73 (m, 1H), 6.70-6.64 (m, 1H), 6.63-6.54 (m, 1H), 6.43-6.35 (m, 1H), 5.57-5.16 (m, 1H), 4.52-4.24 (m, 4H), 3.80-3.69 (m, 1H), 3.59- 3.47 (m, 1H), 3.40-3.31 (m, 1H), 3.28-3.22 (m, 1H), 3.12-3.01 (m, 1H), 2.45-2.36 (m, 4H), 2.32-2.15 (m, 3H), 2.06-1.91 (m, 3H), 1.68- 1.60 (m, 3H). | It was obtained by resolving the compound 59; chiral HPLC resolution conditions: column: ODH (2 × 25 cm); mobile phase: n-heptane/ ethanol (0.1% ammonia water) = 50:50. |
| | 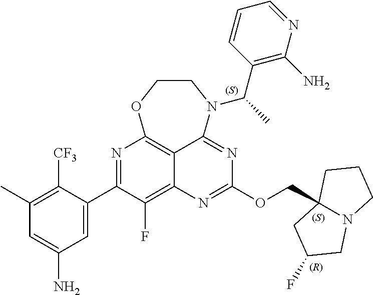 61 & 62 | | | | |
| 62 | | 3-((R or S)-1-(5-(5-amino-3- | 657.4 | (400 MHz, CD₃OD) δ 7.99-7.92 | |
| | | methyl-2- | | (m, 1H), 7.80-7.70 (m, 1H), 6.80- | |
| | | (trifluoromethyl)phenyl)-4- | | 6.74 (m, 1H), 6.68-6.65 (m, 1H), | |
| | | fluoro-2-(((2R,7aS)-2- | | 6.62-6.54 (m, 1H), 6.42-6.36 (m, | |
| | | fluorotetrahydro-1H-pyrrolizin- | | 1H), 5.48-5.22 (m, 1H), 4.53-4.27 | |
| | | 7a(5H)-yl)methoxy)-8,9-dihydro- | | (m, 4H), 3.82-3.68 (m, 1H), 3.60- | |
| | | 10H-7-oxa-1,3,6,10- | | 3.33 (m, 3H), 3.15-3.02 (m, 1H), | |
| | | tetraazacyclohepta[de]naphthalen- | | 2.48-2.35 (m, 4H), 2.33-2.16 (m, | |
| | | 10-yl)ethyl)pyridin-2-amine | | 3H), 2.08-1.88 (m, 3H), 1.69-1.60 | |
| | | (diastereomer 2) | | (m, 3H). | |
| 71 | 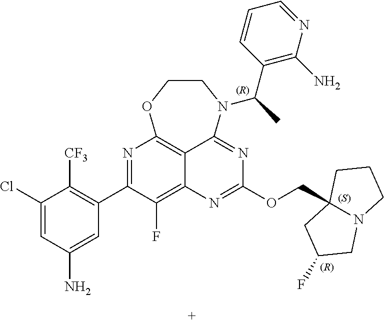 | 3-((R or S)-1-(5-(5-amino-3- chloro-2- (trifluoromethyl)phenyl)-4- fluoro-2-(((2R,7aS)-2- fluorotetrahydro-1H-pyrrolizin- 7a(5H)-yl)methoxy)-8,9-dihydro- 10H-7-oxa-1,3,6,10- tetraazacyclohepta[de]naphthalen- 10-yl)ethyl)pyridin-2-amine (diastereomer 1) | 677.3 | (400 MHz, CD₃OD) δ 7.94 (d, J = 4.7 Hz, 1H), 7.74 (d, J = 7.7 Hz, 1H), 6.86 (d, J = 2.1 Hz, 1H), 6.77 (dd, J = 7.5, 5.2 Hz, 1H), 6.58 (d, J = 7.0 Hz, 1H), 6.46 (s, 1H), 5.39-5.21 (m, 1H), 4.50-4.23 (m, 4H), 3.79-3.47 (m, 2H), 3.28- 3.17 (m, 3H), 3.08-2.94 (m, 1H), 2.40-1.90 (m, 6H), 1.63 (d, J = 6.9 Hz, 3H). | It was obtained by resolving the compound 63; chiral HPLC resolution conditions: column: ODH (2 × 25 cm); mobile phase: n-heptane/ ethanol (0.1% ammonia water) = 50:50. |
| | 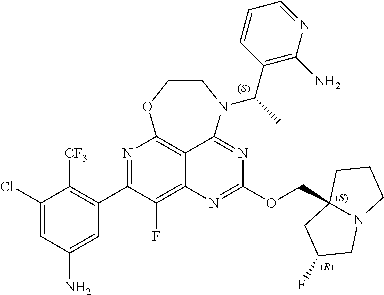 71 & 72 | | | | |
| 72 | | 3-((R or S)-1-(5-(5-amino-3- | 677.3 | (400 MHz, CD₃OD) δ 7.94 (d, J = | |
| | | chloro-2- | | 4.7 Hz, 1H), 7.74 (d, J = 7.7 Hz, | |
| | | (trifluoromethyl)phenyl)-4- | | 1H), 6.86 (d, J = 2.1 Hz, 1H), | |
| | | fluoro-2-(((2R,7aS)-2- | | 6.77 (dd, J = 7.5, 5.2 Hz, 1H), | |
| | | fluorotetrahydro-1H-pyrrolizin- | | 6.58 (d, J = 7.0 Hz, 1H), 6.46 (s, | |
| | | 7a(5H)-yl)methoxy)-8,9-dihydro- | | 1H), 5.39-5.21 (m, 1H), 4.50-4.23 | |
| | | 10H-7-oxa-1,3,6,10- | | (m, 4H), 3.79-3.47 (m, 2H), 3.28- | |
| | | tetraazacyclohepta[de]naphthalen- | | 3.17 (m, 3H), 3.08-2.94 (m, 1H), | |
| | | 10-yl)ethyl)pyridin-2-amine | | 2.40-1.90 (m, 6H), 1.63 (d, J = | |
| | | (diastereomer 2) | | 6.9 Hz, 3H). | |
| 74 | 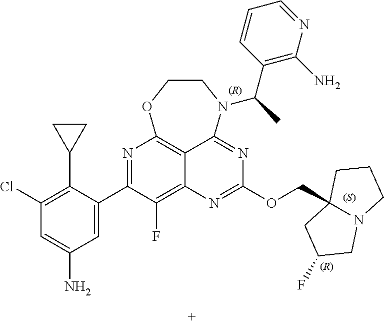 | 3-((R or S)-1-(5-(5-amino-3- chloro-2-cyclopropylphenyl)-4- fluoro-2-(((2R,7aS)-2- fluorotetrahydro-1H-pyrrolizin- 7a(5H)-yl)methoxy)-8,9-dihydro- 10H-7-oxa-1,3,6,10- tetraazacyclohepta[de]naphthalen- 10-yl)ethyl)pyridin-2-amine (diastereomer 1) | 649.3 | (400 MHz, CD₃OD) δ 7.99-7.90 (m, 1H), 7.77-7.72 (m, 1H), 6.84- 6.79 (m, 1H), 6.79-6.74 (m, 1H), 6.63-6.55 (m, 2H), 5.41-5.23 (m, 1H), 4.49-4.40 (m, 1H), 4.37-4.27 (m, 3H), 3.79-3.70 (m, 1H), 3.58 (s, 1H), 3.57-3.50 (m, 1H), 3.37- 3.30 (m, 1H), 3.28-3.21 (m, 2H), 3.09-3.00 (m, 1H), 2.42-2.13 (m, 3H), 2.06-1.88 (m, 3H), 1.83-1.75 (m, 1H), 1.67-1.61 (m, 3H), 0.71- 0.49 (m, 2H), 0.17-0.01 (m, 2H). | It was obtained by resolving the compound prepared from I-A15, I-B1 and I-C3 by following the preparation steps of the compound 2; chiral HPLC resolution conditions: column: ODH (2 × 25 cm); mobile phase: n-heptane/ ethanol (0.1% ammonia water) = 50:50. |
| | 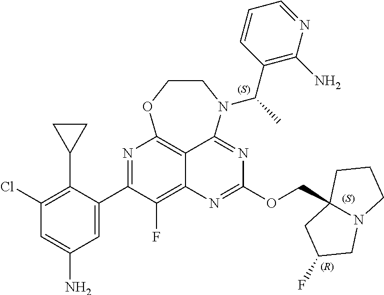 74 & 75 | | | | |
| 75 | | 3-((R or S)-1-(5-(5-amino-3- | 649.3 | (400 MHz, CD₃OD) δ 8.00-7.90 | |
| | | chloro-2-cyclopropylphenyl)-4- | | (m, 1H), 7.78-7.72 (m, 1H), 6.84- | |
| | | fluoro-2-(((2R,7aS)-2- | | 6.80 (m, 1H), 6.80-6.74 (m, 1H), | |
| | | fluorotetrahydro-1H-pyrrolizin- | | 6.63-6.54 (m, 2H), 5.44-5.26 (m, | |
| | | 7a(5H)-yl)methoxy)-8,9-dihydro- | | 1H), 4.50-4.27 (m, 4H), 3.80-3.70 | |
| | | 10H-7-oxa-1,3,6,10- | | (m, 1H), 3.57-3.51 (m, 1H), 3.47- | |
| | | tetraazacyclohepta[de]naphthalen- | | 3.31 (m, 3H), 3.13-3.04 (m, 1H), | |
| | | 10-yl)ethyl)pyridin-2-amine | | 2.47-2.15 (m, 3H), 2.08-1.90 (m, | |
| | | (diastereomer 2) | | 3H), 1.83-1.74 (m, 1H), 1.68-1.61 | |
| | | | | (m, 3H), 0.68-0.52 (m, 2H), 0.15- | |
| | | | | 0.01 (m, 2H). | |
| 76 | 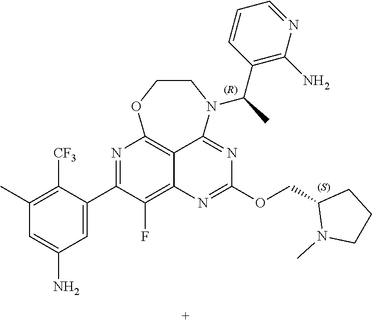 | 3-((R or S)-1-(5-(5-amino-3- methyl-2- (trifluoromethyl)phenyl)-4- fluoro-2-(((S)-1-methylpyrrolidin- 2-yl)methoxy)-8,9-dihydro-10H- 7-oxa-1,3,6,10- tetraazacyclohepta[de]naphthalen- 10-yl)ethyl)pyridin-2-amine (diastereomer 1) | 613.3 | (400 MHz, CD₃OD) δ 7.96-7.92 (m, 1H), 7.74 (d, J = 7.1 Hz, 1H), 7.77-7.71 (m, 1H), 6.80-6.74 (m, 1H), 6.69-6.65 (m, 1H), 6.64-6.56 (m, 1H), 4.52-4.25 (m, 4H), 3.75- 3.67 (m, 1H), 3.58-3.43 (m, 1H), 3.14-3.03 (m, 1H), 2.88-2.73 (m, 1H), 2.51 (s, 3H), 2.45-2.30 (m, 4H), 2.16-2.06 (m, 1H), 1.90-1.73 (m, 3H), 1.68-1.59 (m, 3H). | It was obtained by resolving the compound 65; chiral HPLC resolution conditions: column: IG (2 × 25 cm); mobile phase: n-heptane/ethanol (0.1% ammonia water) = 20:80. |
| | 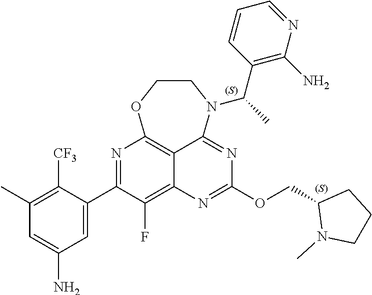 76 & 77 | | | | |
| 77 | | 3-((R or S)-1-(5-(5-amino-3- | 613.3 | (400 MHz, CD₃OD) δ 7.98-7.90 | |
| | | methyl-2- | | (m, 1H), 7.77-7.70 (m, 1H), 6.81- | |
| | | (trifluoromethyl)phenyl)-4- | | 6.73 (m, 1H), 6.69-6.64 (m, 1H), | |
| | | fluoro-2-(((S)-1-methylpyrrolidin- | | 6.63-6.57 (m, 1H), 6.43-6.35 (m, | |
| | | 2-yl)methoxy)-8,9-dihydro-10H- | | 1H), 4.56-4.24 (m, 4H), 3.80-3.66 | |
| | | 7-oxa-1,3,6,10- | | (m, 1H), 3.59-3.45 (m, 1H), 3.14- | |
| | | tetraazacyclohepta[de]naphthalen- | | 3.02 (m, 1H), 2.87-2.74 (m, 1H), | |
| | | 10-yl)ethyl)pyridin-2-amine | | 2.50 (s, 3H), 2.42-2.31 (m, 4H), | |
| | | (diastereomer 2) | | 2.19-2.08 (m, 1H), 1.87-1.72 (m, | |
| | | | | 3H), 1.68-1.59 (m, 3H). | |
| 78 | 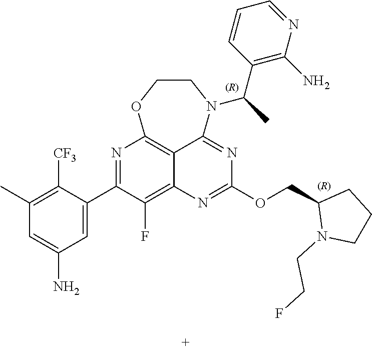 | 3-((R or S)-1-(5-(5-amino-3- methyl-2- (trifluoromethyl)phenyl)-4- fluoro-2-(((R)-1-(2- fluoroethyl)pyrrolidin-2- yl)methoxy)-8,9-dihydro-10H- 7-oxa-1,3,6,10- tetraazacyclohepta[de]naphthalen- 10-yl)ethyl)pyridin-2-amine (diastereomer 1) | 645.3 | (400 MHz, CD₃OD) δ 7.95-7.91 (m, 1H), 7.73 (d, J = 7.5 Hz, 1H), 6.78-7.64 (m, 1H), 6.66 (s, 1H), 6.60 (q, J = 6.8 Hz, 1H), 6.38 (s, 1H), 4.63 (t, J = 4.9 Hz, 1H), 4.55-4.48 (m, 2H), 4.46-4.34 (m, 2H), 4.35-4.25 (m, 1H), 3.76-3.59 (m, 1H), 3.55-3.31 (m, 2H), 3.25- 3.19 (m, 1H), 3.07-3.01 (m, 1H), 2.81-2.65 (m, 1H), 2.50-2.38 (m, 4H), 2.10-1.95 (m, 1H), 1.92-1.68 (m, 3H), 1.63 (d, J = 6.9 Hz, 3H). | It was obtained by resolving the compound prepared from I-A12, I-B11 and I-C3 by following the preparation steps of the compound 2; chiral HPLC resolution conditions: column: IG (2 × 25 cm); mobile phase: n-heptane/ethanol (0.1% ammonia water) = 20:80. |
| | 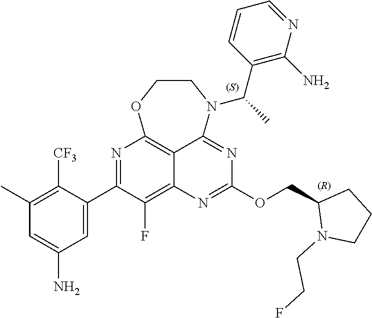 78 & 79 | | | | |
| 79 | | 3-((R or S)-1-(5-(5-amino-3- | 645.3 | (400 MHz, CD3OD) δ 7.94 (d, J = | |
| | | methyl-2- | | 4.7 Hz, 1H), 7.74 (d, J = 7.4 | |
| | | (trifluoromethyl)phenyl)-4- | | Hz, 1H), 6.78-6.74 (m, 1H), 6.70- | |
| | | fluoro-2-(((R)-1-(2- | | 6.55 (m, 2H), 6.38 (s, 1H), 4.66- | |
| | | fluoroethyl)pyrrolidin-2- | | 4.57 (m, 1H), 4.54-4.38 (m, 4H), | |
| | | yl)methoxy)-8,9-dihydro-10H-7- | | 4.35-4.21 (m, 1H), 3.78-3.59 (m, | |
| | | oxa-1,3,6,10- | | 1H), 3.58-3.33 (m, 2H), 3.25-3.19 | |
| | | tetraazacyclohepta[de]naphthalen- | | (m, 1H), 3.09-3.00 (m, 1H), 2.84- | |
| | | 10-yl)ethyl)pyridin-2-amine | | 2.64 (m, 1H), 2.49-2.31 (m, 4H), | |
| | | (diastereomer 2) | | 2.09-2.00 (m, 1H), 1.90-1.69 (m, | |
| | | | | 3H), 1.63 (d, J = 6.9 Hz, 3H). | |
| 80 | 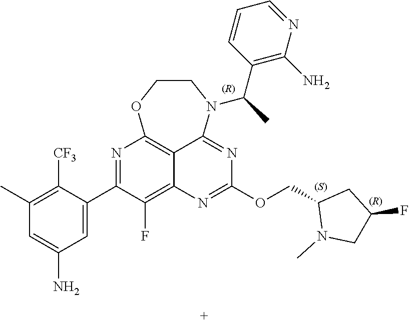 | 3-((R or S)-1-(5-(5-amino-3- methyl-2- (trifluoromethyl)phenyl)-4- fluoro-2-(((2S,4R)-4-fluoro-1- methylpyrrolidin-2-yl)methoxy)- 8,9-dihydro-10H-7-oxa-1,3,6,10- tetraazacyclohepta[de]naphthalen- 10-yl)ethyl)pyridin-2-amine (diastereomer 1) | 631.3 | (400 MHz, CD₃OD) δ 7.94 (d, J = 4.8 Hz, 1H), 7.74 (d, J = 7.4 Hz, 1H), 6.79-6.75 (m, 1H), 6.66 (s, 1H), 6.60 (q, J = 6.7 Hz, 1H), 6.39 (s, 1H), 5.27-5.07 (m, 1H), 4.62-4.48 (m, 2H), 4.47-4.23 (m, 2H), 3.76-3.64 (m, 1H), 3.58-3.42 (m, 2H), 3.20-3.10 (m, 1H), 2.75- 2.53 (m, 4H), 2.40 (s, 3H), 2.36- 2.28 (m, 1H), 2.01-1.91 (m, 1H), 1.63 (d, J = 6.8 Hz, 3H). | It was obtained by resolving the compound 73; chiral HPLC resolution conditions: column: IG (2 × 25 cm); mobile phase: n-heptane/ethanol (0.1% ammonia water) = 20:80. |
| | 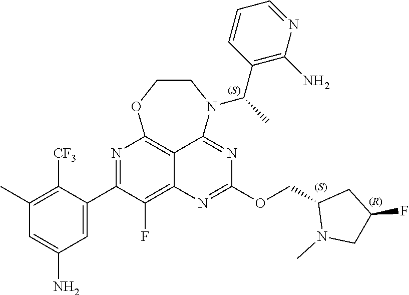 80 & 81 | | | | |
| 81 | | 3-((R or S)-1-(5-(5-amino-3- | 631.3 | (400 MHz, CD₃OD) δ 7.94 (d, J = | |
| | | methyl-2- | | 4.8 Hz, 1H), 7.74 (d, J = 7.4 Hz, | |
| | | (trifluoromethyl)phenyl)-4- | | 1H), 6.78-6.74 (m, 1H), 6.66 (s, | |
| | | fluoro-2-(((2S,4R)-4-fluoro-1- | | 1H), 6.60 (q, J = 6.7 Hz, 1H), | |
| | | methylpyrrolidin-2-yl)methoxy)- | | 6.38 (s, 1H), 5.27-5.08 (m, 1H), | |
| | | 8,9-dihydro-10H-7-oxa-1,3,6,10- | | 4.60-4.48 (m, 2H), 4.47-4.27 (m, | |
| | | tetraazacyclohepta[de]naphthalen- | | 2H), 3.80-3.65 (m, 1H), 3.55-3.45 | |
| | | 10-yl)ethyl)pyridin-2-amine | | (m, 2H), 3.20-3.10 (m, 1H), 2.75- | |
| | | (diastereomer 2) | | 2.50 (m, 4H), 2.40 (d, J = 2.0 Hz, | |
| | | | | 3H), 2.34-2.28 (m, 1H), 2.10-2.06 | |
| | | | | (m, 1H), 1.63 (d, J = 6.8 Hz, 3H). | |
| 85 | 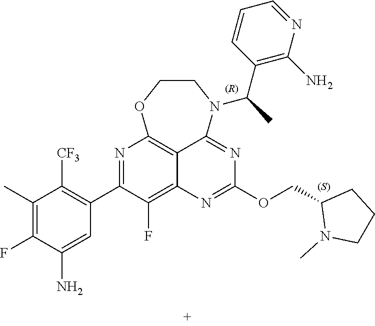 | 3-((R or S)-1-(5-(5-amino-4- fluoro-3-methyl-2- (trifluoromethyl)phenyl)-4- fluoro-2-(((S)-1-methylpyrrolidin- 2-yl)methoxy)-8,9-dihydro-10H- 7-oxa-1,3,6,10- tetraazacyclohepta[de]naphthalen- 10-yl)ethyl)pyridin-2-amine (diastereomer 1) | 631.3 | (400 MHz, CD₃OD) δ 7.98-7.91 (m, 1H), 7.78-7.71 (m, 1H), 6.80- 6.73 (m, 1H), 6.65-6.54 (m, 2H), 4.51 (d, J = 5.6 Hz, 2H), 4.48- 4.25 (m, 2H), 3.78-3.66 (m, 1H), 3.57-3.45 (m, 1H), 3.14-3.03 (m, 1H), 2.89-2.78 (m, 1H), 2.52 (s, 3H), 2.43-2.33 (m, 4H), 2.19-2.05 (m, 1H), 1.88-1.73 (m, 3H), 1.64 (d, J = 6.9 Hz, 3H). | It was obtained by resolving the compound prepared from I-A18, I-B3 and I-C3 by following the preparation steps of the compound 2; chiral HPLC resolution conditions: column: ODH (2 × 25 cm); mobile phase: n-heptane/ethanol (0.1% ammonia water) = 50:50. |
| | 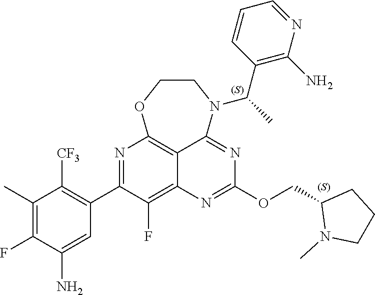 85 & 86 | | | | |
| 86 | | 3-((R or S)-1-(5-(5-amino-4- | 631.3 | (400 MHz, CD₃OD) δ 7.97-7.92 | |
| | | fluoro-3-methyl-2- | | (m, 1H), 7.78-7.70 (m, 1H), 6.81- | |
| | | (trifluoromethyl)phenyl)-4- | | 6.72 (m, 1H), 6.65-6.54 (m, 2H), | |
| | | fluoro-2-(((S)-1-methylpyrrolidin- | | 4.59-4.27 (m, 4H), 3.78-3.67 (m, | |
| | | 2-yl)methoxy)-8,9-dihydro-10H- | | 1H), 3.57-3.46 (m, 1H), 3.14-3.07 | |
| | | 7-oxa-1,3,6,10- | | (m, 1H), 2.88-2.80 (m, 1H), 2.53 | |
| | | tetraazacyclohepta[de]naphthalen- | | (s, 3H), 2.44-2.34 (m, 4H), 2.17- | |
| | | 10-yl)ethyl)pyridin-2-amine | | 2.07 (m, 1H), 1.90-1.76 (m, 3H), | |
| | | (diastereomer 2) | | 1.64 (d, J = 6.9 Hz, 3H). | |
| 88 | 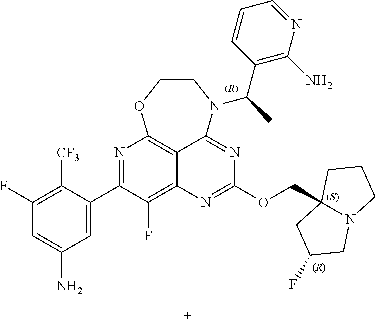 | 3-((R or S)-1-(5-(5-amino-3- fluoro-2- (trifluoromethyl)phenyl)-4- fluoro-2-(((2R,7aS)-2- fluorotetrahydro-1H-pyrrolizin- 7a(5H)-yl)methoxy)-8,9-dihydro- 10H-7-oxa-1,3,6,10- tetraazacyclohepta[de]naphthalen- 10-yl)ethyl)pyridin-2-amine (diastereomer 1) | 661.3 | (400 MHz, CD₃OD) δ 7.98-7.90 (m, 1H), 7.77-7.70 (m, 1H), 6.79- 6.74 (m, 1H), 6.62-6.55 (m, 1H), 6.55-6.49 (m, 1H), 6.40-6.34 (m, 1H), 5.38-5.22 (m, 1H), 4.49-4.39 (m, 1H), 4.37-4.26 (m, 3H), 3.78- 3.69 (m, 1H), 3.57-3.48 (m, 1H), 3.28-3.16 (m, 3H), 3.06-2.96 (m, 1H), 2.39-2.11 (m, 3H), 2.03-1.86 (m, 3H), 1.66-1.60 (m, 3H). | It was obtained by resolving the compound 82; chiral HPLC resolution conditions: column: ODH (2 × 25 cm); mobile phase: n-heptane/ethanol (0.1% ammonia water) = 70:30. |
| | 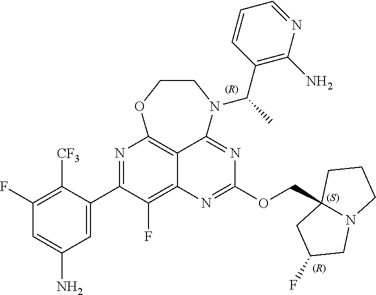 88 & 89 | | | | |
| 89 | | 3-((R or S)-1-(5-(5-amino-3- | 661.3 | (400 MHz, CD₃OD) δ 7.97-7.90 | |
| | | fluoro-2- | | (m, 1H), 7.76-7.71 (m, 1H), 6.80- | |
| | | (trifluoromethyl)phenyl)-4- | | 6.73 (m, 1H), 6.61-6.49 (m, 2H), | |
| | | fluoro-2-(((2R,7aS)-2- | | 6.40-6.35 (m, 1H), 5.39-5.23 (m, | |
| | | fluorotetrahydro-1H-pyrrolizin- | | 1H), 4.48-4.40 (m, 1H), 4.37-4.25 | |
| | | 7a(5H)-yl)methoxy)-8,9-dihydro- | | (m, 3H), 3.78-3.69 (m, 1H), 3.56- | |
| | | 10H-7-oxa-1,3,6,10- | | 3.49 (m, 1H), 3.29-3.18 (m, 3H), | |
| | | tetraazacyclohepta[de]naphthalen- | | 3.07-2.96 (m, 1H), 2.41-2.09 (m, | |
| | | 10-yl)ethyl)pyridin-2-amine | | 3H), 2.05-1.83 (m, 3H), 1.67-1.60 | |
| | | (diastereomer 2) | | (m, 3H). | |
| 90 | 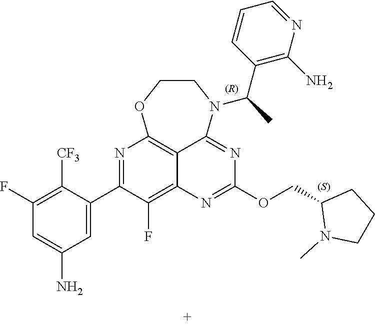 | 3-((R or S)-1-(5-(5-amino-3- fluoro-2- (trifluoromethyl)phenyl)-4- fluoro-2-(((S)-1-methylpyrrolidin- 2-yl)methoxy)-8,9-dihydro-10H- 7-oxa-1,3,6,10- tetraazacyclohepta[de]naphthalen- 10-yl)ethyl)pyridin-2-amine (diastereomer 1) | 617.3 | (400 MHz, CD₃OD) δ 7.97-7.92 (m, 1H), 7.77-7.72 (m, 1H), 6.80- 6.74 (m, 1H), 6.63-6.56 (m, 1H), 6.56-6.48 (m, 1H), 6.40-6.35 (m, 1H), 4.54-4.48 (m, 2H), 4.47-4.39 (m, 1H), 4.36-4.25 (m, 1H), 3.77- 3.66 (m, 1H), 3.55-3.46 (m, 1H), 3.12-3.04 (m, 1H), 2.86-2.76 (m, 1H), 2.51 (s, 3H), 2.39-2.32 (m, 1H), 2.16-2.06 (m, 1H), 1.87-1.72 (m, 3H), 1.67-1.60 (m, 3H). | It was obtained by resolving the compound prepared from I-A16, I-B3 and I-C3 by following the preparation steps of the compound 2; chiral HPLC resolution conditions: column: IG (2 × 25 cm); mobile phase: n-heptane/ethanol (0.1% ammonia water) = 50:50. |
| | 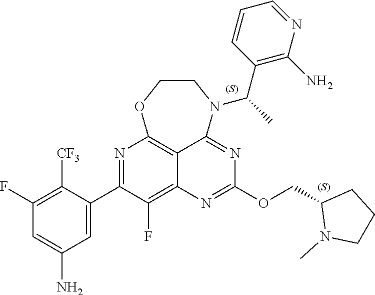 90 & 91 | | | | |
| 91 | | 3-((R or S)-1-(5-(5-amino-3- | 617.3 | (400 MHz, CD₃OD) δ 7.97-7.90 | |
| | | fluoro-2- | | (m, 1H), 7.77-7.72 (m, 1H), 6.79- | |
| | | (trifluoromethyl)phenyl)-4- | | 6.74 (m, 1H), 6.64-6.57 (m, 1H), | |
| | | fluoro-2-(((S)-1-methylpyrrolidin- | | 6.56-6.48 (m, 1H), 6.41-6.35 (m, | |
| | | 2-yl)methoxy)-8,9-dihydro-10H- | | 1H), 4.57-4.51 (m, 1H), 4.50-4.40 | |
| | | 7-oxa-1,3,6,10- | | (m, 2H), 4.36-4.25 (m, 1H), 3.77- | |
| | | tetraazacyclohepta[de]naphthalen- | | 3.68 (m, 1H), 3.56-3.47 (m, 1H), | |
| | | 10-yl)ethyl)pyridin-2-amine | | 3.12-3.03 (m, 1H), 2.84-2.74 (m, | |
| | | (diastereomer 2) | | 1H), 2.50 (s, 3H), 2.38-2.31 (m, | |
| | | | | 1H), 2.14-2.05 (m, 1H), 1.87-1.72 | |
| | | | | (m, 3H), 1.67-1.60 (m, 3H). | |
| 92 | 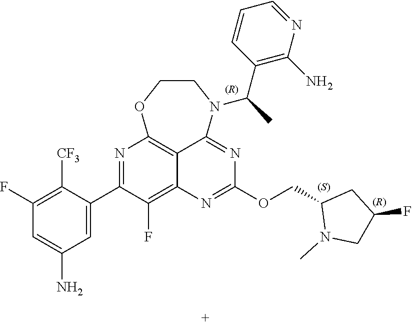 | 3-((R or S)-1-(5-(5-amino-3- fluoro-2- (trifluoromethyl)phenyl)-4- fluoro-2-(((2S,4R)-4-fluoro-1- methylpyrrolidin-2-yl)methoxy)- 8,9-dihydro-10H-7-oxa-1,3,6,10- tetraazacyclohepta[de]naphthalen- 10-yl)ethyl)pyridin-2-amine (diastereomer 1) | 635.3 | (400 MHz, CD₃OD) δ 8.01-7.87 (m, 1H), 7.77-7.72 (m, 1H), 6.80- 6.74 (m, 1H), 6.62-6.56 (m, 1H), 6.55-6.49 (m, 1H), 6.41-6.35 (m, 1H), 5.29-5.09 (m, 1H), 4.64-4.50 (m, 2H), 4.47-4.27 (m, 2H), 3.77- 3.67 (m, 1H), 3.59-3.46 (m, 2H), 3.25-3.17 (m, 1H), 2.76-2.64 (m, 1H), 2.58 (s, 3H), 2.37-2.25 (m, 1H), 2.12-1.95 (m, 1H), 1.67-1.59 (m, 3H). | It was obtained by resolving the compound prepared from I-A16, I-B10 and I-C3 by following the preparation steps of the compound 2; chiral HPLC resolution conditions: column: IG (2 × 25 cm); mobile phase: n-heptane/ethanol (0.1% ammonia water) = 50:50. |
| | 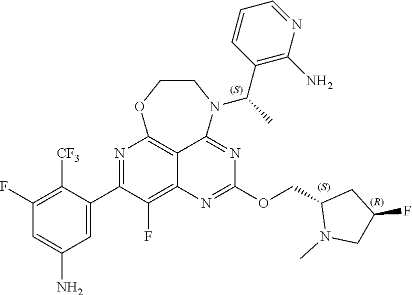 92 & 93 | | | | |
| 93 | | 3-((R or S)-1-(5-(5-amino-3- | 635.3 | (400 MHz, CD₃OD) δ 8.00-7.88 | |
| | | fluoro-2- | | (m, 1H), 7.77-7.71 (m, 1H), 6.81- | |
| | | (trifluoromethyl)phenyl)-4- | | 6.73 (m, 1H), 6.64-6.56 (m, 1H), | |
| | | fluoro-2-(((2S,4R)-4-fluoro-1- | | 6.56-6.48 (m, 1H), 6.42-6.35 (m, | |
| | | methylpyrrolidin-2-yl)methoxy)- | | 1H), 5.28-5.09 (m, 1H), 4.61-4.50 | |
| | | 8,9-dihydro-10H-7-oxa-1,3,6,10- | | (m, 2H), 4.48-4.25 (m, 2H), 3.78- | |
| | | tetraazacyclohepta[de]naphthalen- | | 3.67 (m, 1H), 3.59-3.46 (m, 2H), | |
| | | 10-yl)ethyl)pyridin-2-amine | | 3.23-3.14 (m, 1H), 2.75-2.62 (m, | |
| | | (diastereomer 2) | | 1H), 2.57 (s, 3H), 2.37-2.24 (m, | |
| | | | | 1H), 2.13-1.95 (m, 1H), 1.67-1.60 | |
| | | | | (m, 3H). | |
| 95 | 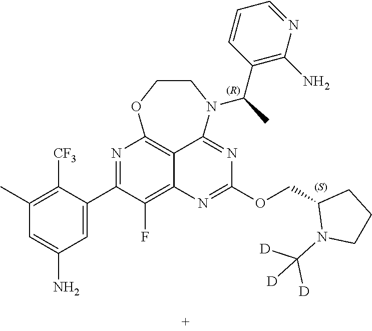 | 3-((R or S)-1-(5-(5-amino-3- methyl-2- (trifluoromethyl)phenyl)-4- fluoro-2-(((S)-1-(methyl- d₃)pyrrolidin-2-yl)methoxy)-8,9- dihydro-10H-7-oxa-1,3,6,10- tetraazacyclohepta[de]naphthalen- 10-yl)ethyl)pyridin-2-amine (diastereomer 1) | 616.3 | (400 MHz, CD₃OD) δ 8.00-7.87 (m, 1H), 7.78-7.71 (m, 1H), 6.81- 6.73 (m, 1H), 6.70-6.63 (m, 1H), 6.63-6.55 (m, 1H), 6.43-6.34 (m, 1H), 4.62-4.50 (m, 2H), 4.47-4.25 (m, 2H), 3.77-3.66 (m, 1H), 3.56- 3.46 (m, 1H), 3.24-3.17 (m, 1H), 3.09-2.98 (m, 1H), 2.58-2.50 (m, 1H), 2.44-2.35 (m, 3H), 2.20-2.11 (m, 1H), 1.93-1.79 (m, 3H), 1.67- 1.59 (m, 3H). | It was obtained by resolving the compound prepared from I-A12, I-B13 and I-C3 by following the preparation steps of the compound 2; chiral HPLC resolution conditions: column: IG (2 × 25 cm); mobile phase: acetonitrile/ethanol (0.1% ammonia water) = 10:90. |
| | 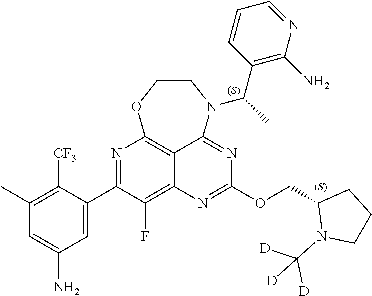 95 & 96 | | | | |
| 96 | | 3-((R or S)-1-(5-(5-amino-3- | 616.4 | (400 MHz, CD₃OD) δ 7.97-7.90 | |
| | | methyl-2- | | (m, 1H), 7.76-7.71 (m, 1H), 6.79- | |
| | | (trifluoromethyl)phenyl)-4- | | 6.74 (m, 1H), 6.68-6.64 (m, 1H), | |
| | | fluoro-2-(((S)-1-(methyl- | | 6.64-6.57 (m, 1H), 6.42-6.35 (m, | |
| | | d₃)pyrrolidin-2-yl)methoxy)-8,9- | | 1H), 4.57-4.38 (m, 3H), 4.35-4.23 | |
| | | dihydro-10H-7-oxa-1,3,6,10- | | (m, 1H), 3.77-3.66 (m, 1H), 3.56- | |
| | | tetraazacyclohepta[de]naphthalen- | | 3.46 (m, 1H), 3.15-3.06 (m, 1H), | |
| | | 10-yl)ethyl)pyridin-2-amine | | 2.89-2.79 (m, 1H), 2.43-2.34 (m, | |
| | | (diastereomer 2) | | 4H), 2.16-2.06 (m, 1H), 1.88-1.74 | |
| | | | | (m, 3H), 1.67-1.60 (m, 3H). | |
| 98 | 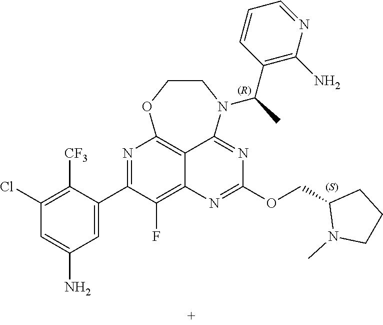 | 3-((R or S)-1-(5-(5-amino-3- chloro-2- (trifluoromethyl)phenyl)-4- fluoro-2-(((S)-1-methylpyrrolidin- 2-yl)methoxy)-8,9-dihydro-10H- 7-oxa-1,3,6,10- tetraazacyclohepta[de]naphthalen- 10-yl)ethyl)pyridin-2-amine (diastereomer 1) | 633.2 | (400 MHz, CD₃OD) δ 7.97-7.90 (m, 1H), 7.74 (d, J = 7.4 Hz, 1H), 6.86 (d, J = 2.1 Hz, 1H), 6.80- 6.73 (m, 1H), 6.63-6.56 (m, 1H), 6.46 (s, 1H), 4.54-4.48 (m, 2H), 4.47-4.37 (m, 1H), 4.36-4.25 (m, 1H), 3.79-3.68 (m, 1H), 3.55-3.45 (m, 1H), 3.12-3.03 (m, 1H), 2.84- 2.75 (m, 1H), 2.50 (s, 3H), 2.39- 2.31 (m, 1H), 2.19-2.07 (m, 1H), 1.89-1.73 (m, 3H), 1.63 (d, J = 6.9 Hz, 3H). | It was obtained by resolving the compound prepared from I-A14, I-B3 and I-C3 by following the preparation steps of the compound 2; chiral HPLC resolution conditions: column: IG (2 × 25 cm); mobile phase: ethanol (0.1% ammonia water). |
| | 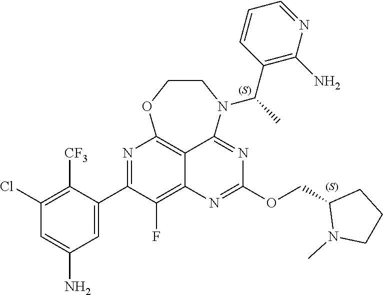 98 & 99 | | | | |
| 99 | | 3-((R or S)-1-(5-(5-amino-3- | 633.2 | (400 MHz, CD₃OD) δ 7.97-7.91 | |
| | | chloro-2- | | (m, 1H), 7.74 (d, J = 7.5 Hz, 1H), | |
| | | (trifluoromethyl)phenyl)-4- | | 6.86 (d, J = 2.1 Hz, 1H), 6.79- | |
| | | fluoro-2-(((S)-1-methylpyrrolidin- | | 6.73 (m, 1H), 6.65-6.57 (m, 1H), | |
| | | 2-yl)methoxy)-8,9-dihydro-10H- | | 6.46 (s, 1H), 4.59-4.51 (m, 1H), | |
| | | 7-oxa-1,3,6,10- | | 4.50-4.39 (m, 2H), 4.36-4.25 (m, | |
| | | tetraazacyclohepta[de]naphthalen- | | 1H), 3.78-3.67 (m, 1H), 3.59-3.46 | |
| | | 10-yl)ethyl)pyridin-2-amine | | (m, 1H), 3.12-3.04 (m, 1H), 2.85- | |
| | | (diastereomer 2) | | 2.75 (m, 1H), 2.50 (s, 3H), 2.39- | |
| | | | | 2.30 (m, 1H), 2.18-2.06 (m, 1H), | |
| | | | | 1.88-1.75 (m, 3H), 1.63 (d, J = | |
| | | | | 6.9 Hz, 3H). | |
| 100 | 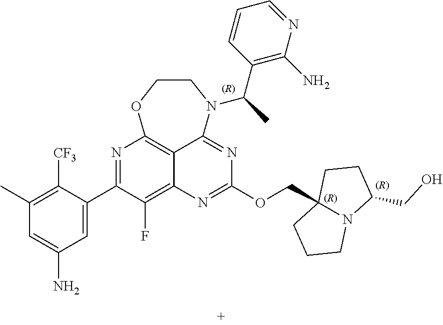 | ((3R,7aR)-7a-(((5-(5-amino-3- methyl-2- (trifluoromethyl)phenyl)-10-((R or S)-1-(2-aminopyridin-3- yl)ethyl)-4-fluoro-9,10-dihydro- 8H-7-oxa-1,3,6,10- tetraazacyclohepta[de]naphthalen- 2-yl)oxy)methyl)hexahydro-1H- pyrrrolizin-3-yl)methanol (diastereomer 1) | 669.4 | (400 MHz, DMSO-d₆) δ 7.96 (d, J = 5.0 Hz, 1H), 7.80-7.73 (m, 1H), 6.87-6.79 (m, 1H), 6.71 (s, 1H), 6.54 (d, J = 6.5 Hz, 1H), 6.40 (s, 1H), 4.06-3.74 (m, 6H), 3.69- 3.60 (m, 4H), 3.39 (s, 1H), 2.40 (s, 5H), 2.25-1.96 (m, 6H), 1.65 (d, J = 6.8 Hz, 3H). | It was obtained by resolving the compound prepared from I-A12, 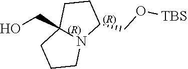 I-B14 and I-C3 by following the preparation steps of the compound 2; chiral HPLC resolution conditions: column: ODH (2 × 25 cm); mobile phase: n-heptane/ethanol (0.1% ammonia water) = 50:50. |
| | 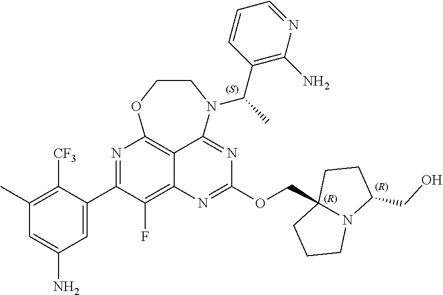 100 & 101 | | | | |
| 101 | | ((3R,7aR)-7a-(((5-(5-amino-3- | 669.4 | (400 MHz, DMSO-d₆) δ 8.39 (s, | |
| | | methyl-2- | | 2H), 7.34-7.26 (m, 5H), 6.23 (d, J = | |
| | | (trifluoromethyl)phenyl)-10-((R | | 8.2 Hz, 1H), 5.66 (d, J = 6.9 | |
| | | or S)-1-(2-aminopyridin-3- | | Hz, 1H), 4.69-4.52 (m, 5H), 4.17- | |
| | | yl)ethyl)-4-fluoro-9,10-dihydro- | | 3.81 (m, 4H), 3.67 (s, 1H), 3.55- | |
| | | 8H-7-oxa-1,3,6,10- | | 3.14 (m, 18H), 2.99-2.91 (m, 5H), | |
| | | tetraazacyclohepta[de]naphthalen- | | 2.54 (d, J = 4.9 Hz, 3H), 2.45 (s, | |
| | | 2-yl)oxy)methyl)hexahydro-1H- | | 5H), 2.36-2.22 (m, 1H), 1.98-1.92 | |
| | | pyrrolizin-3-yl)methanol | | (m, 7H), 1.22 (s, 1H), 1.09 (d, J = | |
| | | (diastereomer 2) | | 6.6 Hz, 3H), 0.97-0.84 (m, 19H), | |
| | | | | 0.79-0.75 (m, 4H). | |
| 102 | 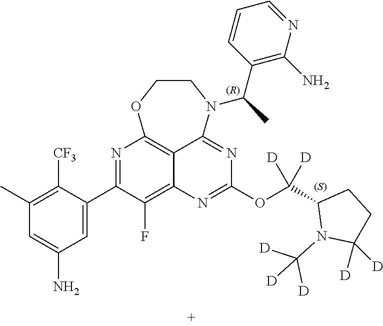 | 3-((R or S)-1-(5-(5-amino-3- methyl-2- (trifluoromethyl)phenyl)-4- fluoro-2-(((S)-1-(methyl- d₃)pyrrolidin-2-yl-5,5- d₂)methoxy-d₂)-8,9-dihydro-10H- 7-oxa-1,3,6,10- tetraazacyclohepta[de]naphthalen- 10-yl)ethyl)pyridin-2-amine (diastereomer 2) | 620.4 | (400 MHz, CD₃OD) δ 7.97-7.92 (m, 1H), 7.77-7.73 (m, 1H), 6.80- 6.75 (m, 1H), 6.68-6.64 (m, 1H), 6.63-6.57 (m, 1H), 6.41-6.36 (m, 1H), 4.47-4.25 (m, 2H), 3.76-3.68 (m, 1H), 3.56-3.46 (m, 1H), 2.89- 2.81 (m, 1H), 2.43-2.37 (m, 3H), 2.18-2.07 (m, 1H), 1.86-1.74 (m, 3H), 1.66-1.61 (m, 3H). | It was obtained by resolving the compound prepared from I-A12, I-B15 and I-C3 by following the preparation steps of the compound 2; chiral HPLC resolution conditions: column: IG (2 × 25 cm); mobile phase: acetonitrile/ ethanol (0.1% ammonia water) = 10:90. |
| | 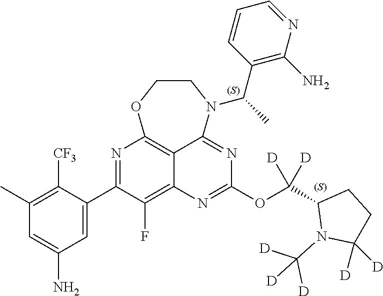 102 & 103 | | | | |
| 103 | | 3-((R or S)-1-(5-(5-amino-3- | 620.4 | (400 MHz, CD₃OD) δ 7.97-7.92 | |
| | | methyl-2- | | (m, 1H), 7.77-7.72 (m, 1H), 6.80- | |
| | | (trifluoromethyl)phenyl)-4- | | 6.75 (m, 1H), 6.69-6.65 (m, 1H), | |
| | | fluoro-2-(((S)-1-(methyl- | | 6.64-6.56 (m, 1H), 6.41-6.36 (m, | |
| | | d₃)pyrrolidin-2-yl-5,5- | | 1H), 4.50-4.23 (m, 2H), 3.78-3.68 | |
| | | d₂)methoxy-d₂)-8,9-dihydro-10H- | | (m, 1H), 3.56-3.46 (m, 1H), 2.93- | |
| | | 7-oxa-1,3,6,10- | | 2.85 (m, 1H), 2.43-2.38 (m, 3H), | |
| | | tetraazacyclohepta[de]naphthalen- | | 2.17-2.09 (m, 1H), 1.90-1.74 (m, | |
| | | 10-yl)ethyl)pyridin-2-amine | | 3H), 1.66-1.61 (m, 3H). | |
| | | (diastereomer 2) | | | |
| 109 | 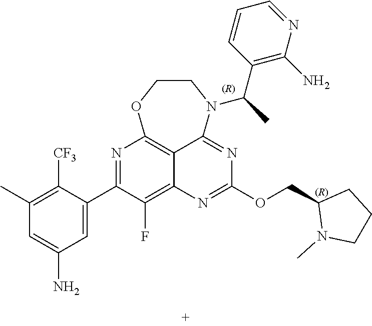 | 3-((R or S)-1-(5-(5-amino-3- methyl-2- (trifluoromethyl)phenyl)-4- fluoro-2-(((R)-1- methylpyrrolidin-2-yl)methoxy)- 8,9-dihydro-10H-7-oxa-1,3,6,10- tetraazacyclohepta[de]naphthalen- 10-yl)ethyl)pyridin-2-amine (diastereomer 1) | 613.4 | (400 MHz, CD₃OD) δ 7.99-7.90 (m, 1H), 7.78-7.71 (m, 1H), 6.80- 6.74 (m, 1H), 6.69-6.65 (m, 1H), 6.64-6.57 (m, 1H), 6.42-6.35 (m, 1H), 4.57-4.24 (m, 4H), 3.82-3.68 (m, 1H), 3.58-3.42 (m, 1H), 3.17- 3.07 (m, 1H), 2.92-2.77 (m, 1H), 2.53 (s, 3H), 2.44-2.32 (m, 4H), 2.21-2.08 (m, 1H), 1.93-1.77 (m, 3H), 1.67-1.59 (m, 3H). | It was obtained by resolving the compound prepared from I-A12, 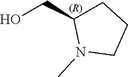 I-B19 and I-C3 by following the preparation steps of the compound 2; chiral HPLC resolution conditions: column: IG (2 × 25 cm); mobile phase: n-heptane/ ethanol (0.1% ammonia water) = 20:80. |
| | 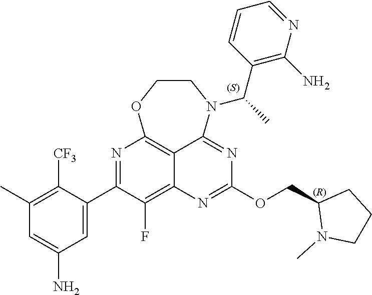 109 & 110 | | | | |
| 110 | | 3-((R or S)-1-(5-(5-amino-3- | 613.3 | (400 MHz, CD₃OD) δ 7.98-7.91 | |
| | | methyl-2- | | (m, 1H), 7.79-7.72 (m, 1H), | |
| | | (trifluoromethyl)phenyl)-4- | | 6..81-6.74 (m, 1H), 6.69-6.65 (m, | |
| | | fluoro-2-(((R)-1- | | 1H), 6.63-6.55 (m, 1H), 6.42-6.35 | |
| | | methylpyrrolidin-2-yl)methoxy)- | | (m, 1H), 4.61-4.27 (m, 4H), 3.78- | |
| | | 8,9-dihydro-10H-7-oxa-1,3,6,10- | | 3.66 (m, 1H), 3.60-3.45 (m, 1H), | |
| | | tetraazacyclohepta[de]naphthalen- | | 3.25-3.18 (m, 1H), 3.10-2.97 (m, | |
| | | 10-yl)ethyl)pyridin-2-amine | | 1H), 2.63 (s, 3H), 2.59-2.47 (m, | |
| | | (diastereomer 2) | | 1H), 2.44-2.36 (m, 3H), 2.22-2.14 | |
| | | | | (m, 1H), 1.94-1.79 (m, 3H), 1.69- | |
| | | | | 1.59 (m, 3H). | |
| 115 | 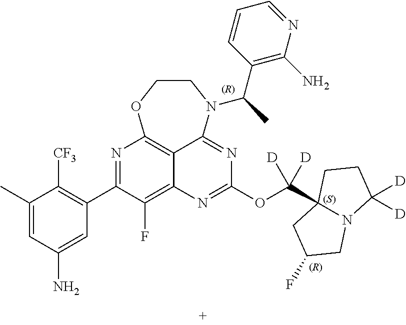 | 3-((R or S)-1-(5-(5-amino-3- methyl-2- (trifluoromethyl)phenyl)-4- fluoro-2-(((2R,7aS)-2- fluorotetrahydro-1H-pyrrolizin- 7a(5H)-yl-5,5-d₂)methoxy-d₂)- 8,9-dihydro-10H-7-oxa-1,3,6,10- tetraazacyclohepta[de]naphthalen- 10-yl)ethyl)pyridin-2-amine (diastereomer 1) | 661.4 | (400 MHz, CD₃OD) δ 7.96-7.92 (m, 1H), 7.73 (d, J = 7.2 Hz, 1H), 6.79-6.72 (m, 1H), 6.66 (s, 1H), 6.59 (q, J = 6.8 Hz, 1H), 6.38 (s, 1H), 5.40-5.20 (m, 1H), 4.50-4.22 (m, 2H), 3.78-3.66 (m, 1H), 3.55- 3.45 (m, 1H), 3.27-3.13 (m, 2H), 2.44-2.08 (m, 6H), 2.05-1.76 (m, 3H), 1.63 (d, J = 6.9 Hz, 3H). | It was obtained by resolving the compound 105; chiral HPLC resolution conditions: column: ODH (2 × 25 cm); mobile phase: n-heptane/ ethanol (0.1% ammonia water) = 50:50. |
| | 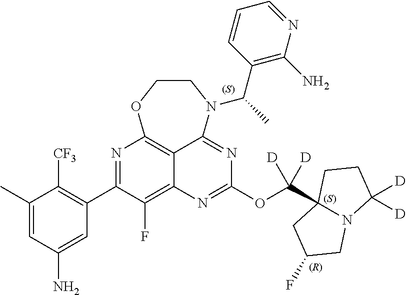 115 & 116 | | | | |
| 116 | | 3-((R or S)-1-(5-(5-amino-3- | 661.4 | (400 MHz, CD₃OD) δ 7.96-7.90 | |
| | | methyl-2- | | (m, J = 4.6 Hz, 1H), 7.72 (d, J = | |
| | | (trifluoromethyl)phenyl)-4- | | 7.4 Hz, 1H), 6.78-6.72 (m, 1H), | |
| | | fluoro-2-(((2R,7aS)-2- | | 6.66 (s, 1H), 6.58 (q, J = 6.5 Hz, | |
| | | fluorotetrahydro-1H-pyrrolizin- | | 1H), 6.39 (s, 1H), 5.42-5.19 (m, | |
| | | 7a(5H)-yl-5,5-d₂)methoxy-d₂)- | | 1H), 4.50-4.19 (m, 2H), 3.78-3.61 | |
| | | 8,9-dihydro-10H-7-oxa-1,3,6,10- | | (m, 1H), 3.56-3.43 (m, 1H), 3.33- | |
| | | tetraazacyclohepta[de]naphthalen- | | 3.12 (m, 2H), 2.43-2.06 (m, 6H), | |
| | | 10-yl)ethyl)pyridin-2-amine | | 2.03-1.78 (m, 3H), 1.62 (d, J = | |
| | | (diastereomer 2) | | 6.9 Hz, 3H). | |
| 117 | 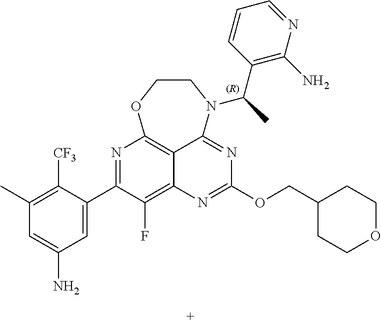 | (R or S)-3-(1-(5-(5-amino-3- methyl-2- (trifluoromethyl)phenyl)-4- fluoro-2-((tetrahydro-2H-pyran-4- yl)methoxy)-8,9-dihydro-10H- 7-oxa-1,3,6,10- tetraazacyclohepta[de]naphthalen- 10-yl)ethyl)pyridin-2-amine (enantiomer 1) | 614.3 | (400 MHz, CD₃OD) δ 7.98-7.92 (m, 1H), 7.78-7.70 (m, 1H), 6.81- 6.73 (m, 1H), 6.69-6.64 (m, 1H), 6.62-6.54 (m, 1H), 6.41-6.35 (m, 1H), 4.49-4.24 (m, 4H), 4.06-3.92 (m, 2H), 3.80-3.67 (m, 1H), 3.57- 3.40 (m, 3H), 2.44-2.37 (m, 3H), 2.21-2.11 (m, 1H), 1.87-1.74 (m, 2H), 1.67-1.61 (m, 3H), 1.54-1.43 (m, 2H). | It was obtained by resolving the compound prepared from I-A12, 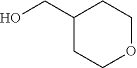 I-B21 and I-C3 by following the preparation steps of the compound 2; chiral HPLC resolution conditions: column: IG (2 × 25 cm); mobile phase: n-heptane/ethanol (0.1% ammonia water) = 20:80. |
| | 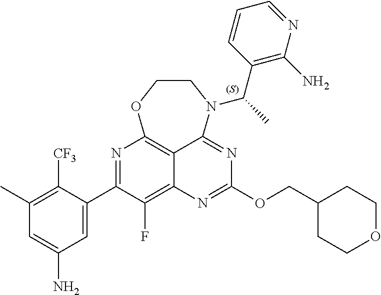 117 & 118 | | | | |
| 118 | | (R or S)-3-(1-(5-(5-amino-3- | 614.4 | (400 MHz, CD₃OD) δ 7.98-7.91 | |
| | | methyl-2- | | (m, 1H), 7.76-7.70 (m, 1H), 6.80- | |
| | | (trifluoromethyl)phenyl)-4- | | 6.73 (m, 1H), 6.68-6.64 (m, 1H), | |
| | | fluoro-2-((tetrahydro-2H-pyran-4- | | 6.62-6.55 (m, 1H), 6.42-6.36 (m, | |
| | | yl)methoxy)-8,9-dihydro-10H-7- | | 1H), 4.47-4.25 (m, 4H), 4.02-3.92 | |
| | | oxa-1,3,6,10- | | (m, 2H), 3.75-3.65 (m, 1H), 3.56- | |
| | | tetraazacyclohepta[de]napthalen- | | 3.40 (m, 3H), 2.44-2.36 (m, 3H), | |
| | | 10-yl)ethyl)pyridin-2-amine | | 2.20-2.08 (m, 1H), 2.22-2.08 (m, | |
| | | (enantiomer 2) | | 2H), 1.84-1.74 (m, 3H), 1.70-1.60 | |
| | | | | (m, 2H). | |
| 124 | 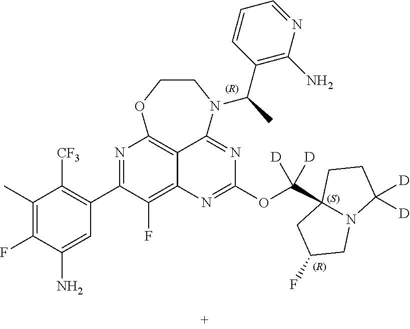 | 3-((R or S)-1-(5-(5-amino-4- fluoro-3-methyl-2- (trifluoromethyl)phenyl)-4- fluoro-2-(((2R,7aS)-2- fluorotetrahydro-1H-pyrrolizin- 7a(5H)-yl-5,5-d₂)methoxy-d₂)- 8,9-dihydro-10H-7-oxa-1,3,6,10- tetraazacyclohepta[de]naphthalen- 10-yl)ethyl)pyridin-2-amine (diastereomer 1) | 679.3 | (400 MHz, CD₃OD) δ 7.98-7.90 (m, 1H), 7.76-7.72 (m, 1H), 6.79- 6.74 (m, 1H), 6.62-6.54 (m, 2H), 5.41-5.24 (m, 1H), 4.52-4.20 (m, 2H), 3.79-3.68 (m, 1H), 3.57-3.48 (m, 1H), 3.40-3.31 (m, 1H), 3.28- 3.24 (m, 1H), 2.43-2.13 (m, 6H), 2.07-1.89 (m, 3H), 1.66-1.61 (m, 3H). | It was obtained by resolving the compound 119; chiral HPLC resolution conditions: column: ODH (2 × 25 cm); mobile phase: n-heptane/ ethanol (0.1% ammonia water) = 20:80. |
| | 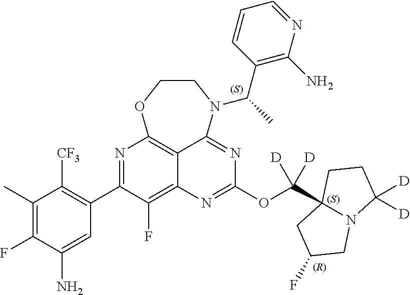 124 & 125 | | | | |
| 125 | | 3-((R or S)-1-(5-(5-amino-4- | 679.3 | (400 MHz, CD₃OD) δ 7.98-7.90 | |
| | | fluoro-3-methyl-2- | | (m, 1H), 7.76-7.72 (m, 1H), 6.79- | |
| | | (trifluoromehtyl)phenyl)-4- | | 6.74 (m, 1H), 6.63-6.52 (m, 2H), | |
| | | fluoro-2-(((2R,7aS)-2- | | 5.41-5.22 (m, 1H), 4.49-4.24 (m, | |
| | | fluorotetrahydro-1H-pyrrolizin- | | 2H), 3.79-3.68 (m, 1H), 3.57-3.46 | |
| | | 7a(5H)-yl-5,5-d₂)methoxy-d₂)- | | (m, 1H), 3.37-3.20 (m, 2H), 2.42- | |
| | | 8,9-dihydro-10H-7-oxa-1,3,6,10- | | 2.09 (m, 6H), 2.05-1.83 (m, 3H), | |
| | | tetraazacyclohepta[de]naphthalen- | | 1.67-1.59 (m, 3H). | |
| | | 10-yl)ethyl)pyridin-2-amine | | | |
| | | (diastereomer 2) | | | |
| 127 | 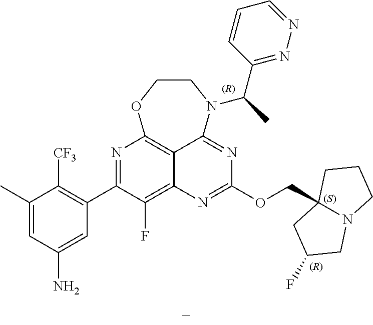 | 3-(4-fluoro-2-(((2R,7aS)-2- fluorotetrahydro-1H-pyrrolizin- 7a(5H)-yl)methoxy)-10-((R or S)- 1-(pyridazin-3-yl)ethyl)-9,10- dihydro-8H-7-oxa-1,3,6,10- tetraazacyclohepta[de]naphthalen- 5-yl)-5-methyl-4- (trifluoromethyl)aniline (diastereomer 1) | 643.3 | (400 MHz, CD₃OD) δ 9.15-9.08 (d, J = 3.7 Hz, 1H), 7.86-7.79 (m, 1H), 7.73-7.68 (m, 1H), 6.70-6.56 (m, 2H), 6.44-6.36 (m, 1H), 5.36- 5.17 (m, 1H), 4.68-4.53 (m, 2H), 4.12-3.84 (m, 4H), 3.27-3.10 (m, 3H), 3.02-2.92 (m, 1H), 2.31-2.00 (m, 3H), 1.97-1.76 (m, 6H). | It was obtained by resolving the compound 121; chiral HPLC resolution conditions: column: ODH (2 × 25 cm); mobile phase: n-heptane/ethanol (0.1% ammonia water) = 20:80. |
| | 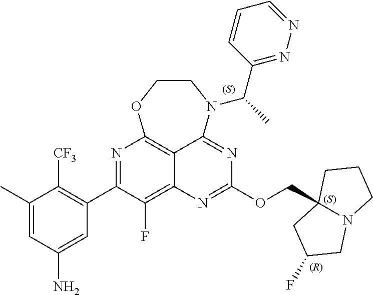 127 & 128 | | | | |
| 128 | | 3-(4-fluoro-2-(((2R,7aS)-2- | 643.3 | (400 MHz, CD₃OD) δ 9.16-9.08 | |
| | | fluorotetrahydro-1H-pyrrolizin- | | (m, 1H), 7.87-7.79 (m, 1H), 7.73- | |
| | | 7a(5H)-yl)methoxy)-10-((R or S)- | | 7.68 (m, 1H), 6.70-6.56 (m, 2H), | |
| | | 1-(pyridazin-3-yl)ethyl)-9,10- | | 6.44-6.36 (m, 1H), 5.35-5.15 (m, | |
| | | dihydro-8H-7-oxa-1,3,6,10- | | 1H), 4.70-4.52 (m, 2H), 4.14-3.86 | |
| | | tetraazacyclohepta[de]naphthalen- | | (m, 4H), 3.27-3.11 (m, 3H), 3.03- | |
| | | 5-yl)-5-methyl-4- | | 2.91 (m, 1H), 2.44-2.36 (m, 3H), | |
| | | (trifluoromethyl)aniline | | 2.31-2.01 (m, 3H), 2.00-1.76 (m, | |
| | | (diastereomer 2) | | 6H). | |
| 130 | 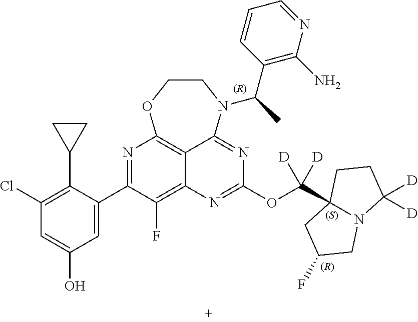 | 3-(10-((R or S)-1-(2- aminopyridin-3-yl)ethyl)-4- fluoro-2-(((2R,7aS)-2- fluorotetrahydro-1H-pyrrolizin- 7a(5H)-yl-5,5-d₂)methoxy-d₂)- 9,10-dihydro-8H-7-oxa-1,3,6,10- tetraazacyclohepta[de]naphthalen- 5-yl)-5-chloro-4- cyclopropylphenyl(diastereomer 1) | 654.3 | (400 MHz, CD₃OD) δ 7.99-7.91 (m, 1H), 7.79-7.71 (m, 1H), 6.94- 6.89 (m, 1H), 6.81-6.75 (m, 1H), 6.74-6.70 (m, 1H), 6.65-6.55 (m, 1H), 5.47-5.16 (m, 1H), 4.55-4.23 (m, 2H), 3.83-3.69 (m, 1H), 3.62- 3.49 (m, 1H), 3.28-3.15 (m, 2H), 2.41-2.08 (m, 3H), 2.01-1.93 (m, 2H), 1.91-1.78 (m, 2H), 1.71-1.60 (m, 3H), 0.73-0.57 (m, 2H), 0.24- 0.01 (m, 2H). | It was obtained by resolving the compound prepared from I-A3, I-B16 and I-C3 by following the preparation steps of the compound 2; chiral HPLC resolution conditions: column: IG (2 × 25 cm); mobile phase: ethanol (0.1% ammonia water). |
| | 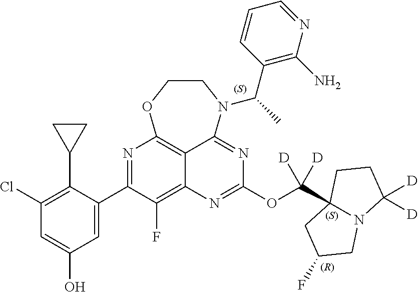 130 & 131 | | | | |
| 131 | | 3-(10-((R or S)-1-(2- | 654.3 | (400 MHz, CD₃OD) δ 7.97-7.92 | |
| | | aminopyridin-3-yl)ethyl)-4- | | (m, 1H), 7.78-7.71 (m, 1H), 6.85- | |
| | | fluoro-2-(((2R,7aS)-2- | | 6.81 (m, 1H), 6.80-6.73 (m, 1H), | |
| | | fluorotetrahydro-1H-pyrrolizin- | | 6.66-6.63 (m, 1H), 6.62-6.55 (m, | |
| | | 7a(5H)-yl-5,5-d₂)methoxy-d₂)- | | 1H), 5.50-5.15 (m, 1H), 4.54-4.21 | |
| | | 9,10-dihydro-8H-7-oxa-1,3,6,10- | | (m, 2H), 3.87-3.69 (m, 1H), 3.63- | |
| | | tetraazacyclohepta[de]naphthalen- | | 3.49 (m, 1H), 3.27-3.12 (m, 2H), | |
| | | 5-yl)-5-chloro-4- | | 2.38-2.12 (m, 3H), 2.06-1.78 (m, | |
| | | cyclopropylphenol (diastereomer 2) | | 4H), 1.70-1.60 (m, 3H), 0.68-0.54 | |
| | | | | (m, 2H), 0.18-0.04 (m, 2H). | |
| 132 | 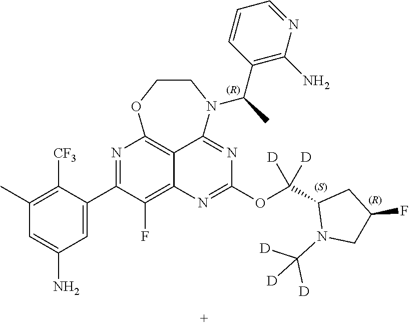 | 3-((R or S)-1-(5-(5-amino-3- methyl-2- (trifluoromethyl)phenyl)-4- fluoro-2-(((2S,4R)-4-fluoro-1- (methyl-d₃)pyrrolidin-2- yl)methoxy-d₂)-8,9-dihydro-10H- 7-oxa-1,3,6,10- tetraazacyclohepta[de]naphthalen- 10-yl)ethyl)pyridin-2-amine (diastereomer 1) | 636.3 | (400 MHz, CD₃OD) δ 8.01-7.86 (m, 1H), 7.74 (d, J = 7.5 Hz, 1H), 6.81-6.72 (m, 1H), 6.66-6.64 (m, 1H), 6.63-6.55 (m, 1H), 6.38 (s, 1H), 5.28-5.03 (m, 1H), 4.49-4.22 (m, 2H), 3.78-3.65 (m, 1H), 3.57- 3.42 (m, 2H), 3.20-3.10 (m, 1H), 2.72-2.56 (m, 1H), 2.43-2.37 (m, 3H), 2.36-2.22 (m, 1H), 2.10-1.91 (m, 1H), 1.63 (d, J = 6.9 Hz, 3H). | It was obtained by resolving the compound 122; chiral HPLC resolution conditions: column: IG (2 × 25 cm); mobile phase: n-heptane/ethanol (0.1% ammonia water) = 20:80. |
| | 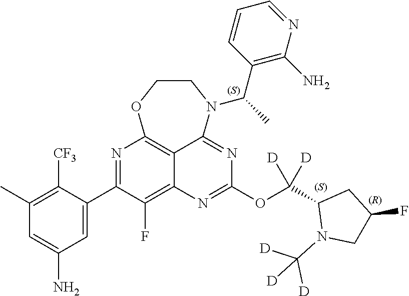 132 & 133 | | | | |
| 133 | | 3-((R or S)-1-(5-(5-amino-3- | 636.3 | (400 MHz, CD₃OD) δ 8.01-7.86 | |
| | | methyl-2- | | (m, 1H), 7.74 (d, J = 7.5 Hz, 1H), | |
| | | (trifluoromethyl)phenyl)-4- | | 6.81-6.72 (m, 1H), 6.66-6.64 (m, | |
| | | fluoro-2-(((2S,4R)-4-fluoro-1- | | 1H), 6.63-6.55 (m, 1H), 6.38 (s, | |
| | | (methyl-d₃)pyrrolidin-2- | | 1H), 5.28-5.03 (m, 1H), 4.49-4.22 | |
| | | yl)methoxy-d₂)-8,9-dihydro-10H- | | (m, 2H), 3.78-3.65 (m, 1H), 3.57- | |
| | | 7-oxa-1,3,6,10- | | 3.42 (m, 2H), 3.20-3.10 (m, 1H), | |
| | | tetraazacyclohepta[de]naphthalen- | | 2.72-2.56 (m, 1H), 2.43-2.37 (m, | |
| | | 10-yl)ethyl)pyridin-2-amine | | 3H), 2.36-2.22 (m, 1H), 2.10-1.91 | |
| | | (diastereomer 2) | | (m, 1H), 1.63 (d, J = 6.9 Hz, 3H). | |
| 137 | 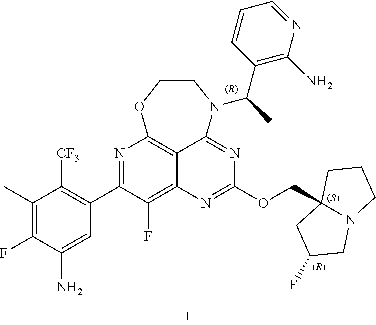 | 3-((R os S)-1-(5-(5-amino-4- fluoro-3-methyl-2- (trifluoromethyl)phenyl)-4- fluoro-2-(((2R,7aS)-2- fluorotetrahydro-1H-pyrrolizin- 7a(5H)-yl)methoxy)-8,9-dihydro- 10H-7-oxa-1,3,6,10- tetraazacyclohepta[de]naphthalen- 10-yl)ethyl)pyridin-2-amine (diastereomer 1) | 675.3 | (400 MHz, CD₃OD) δ 7.99-7.89 (m, 1H), 7.76-7.69 (m, 1H), 6.79- 6.71 (m, 1H), 6.64-6.53 (m, 2H), 5.40-5.19 (m, 1H), 4.50-4.23 (m, 4H), 3.79-3.65 (m, 1H), 3.56-3.46 (m, 1H), 3.29-3.13 (m, 3H), 3.05- 2.95 (m, 1H), 2.39-2.10 (m, 6H), 2.03-1.84 (m, 3H), 1.68-1.57 (m, 3H). | It was obtained by resolving the compound 120; chiral HPLC resolution conditions: column: ODH (2 × 25 cm); mobile phase: n-heptane/ ethanol (0.1% ammonia water) = 20:80. |
| | 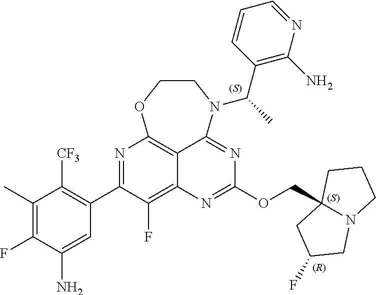 137 & 138 | | | | |
| 138 | | 3-((R or S)-1-(5-(5-amino-4- | 675.3 | (400 MHz, CD₃OD) δ 7.99-7.88 | |
| | | fluoro-3-methyl-2- | | (m, 1H), 7.76-7.69 (m, 1H), 6.79- | |
| | | (trifluoromethyl)phenyl)-4- | | 6.72 (m, 1H), 6.65-6.49 (m, 2H), | |
| | | fluoro-2-(((2R,7aS)-2- | | 5.39-5.21 (m, 1H), 4.48-4.23 (m, | |
| | | fluorotetrahydro-1H-pyrrolizin- | | 4H), 3.78-3.65 (m, 1H), 3.56-3.47 | |
| | | 7a(5H)-yl)methoxy)-8,9-dihydro- | | (m, 1H), 3.29-3.14 (m, 3H), 3.06- | |
| | | 10H-7-oxa-1,3,6,10- | | 2.95 (m, 1H), 2.40-2.09 (m, 6H), | |
| | | tetraazacyclohepta[de]naphthalen- | | 2.03-1.83 (m, 3H), 1.67-1.58 (m, | |
| | | 10-yl)ethyl)pyridin-2-amine | | 3H). | |
| | | (diasetereomer 2) | | | |
| 142 | 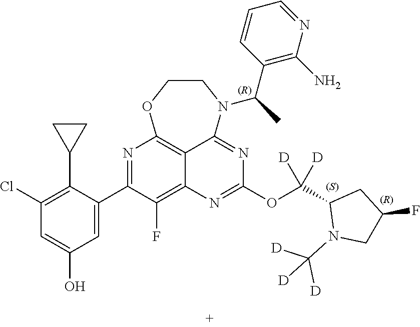 | 3-(10-((R or S)-1-(2- aminopyridin-3-yl)ethyl)-4- fluoro-2-(((2S,4R)-4-fluoro-1- (methyl-d₃)pyrrolidin-2- yl)methoxy-d₂)-9,10-dihydro-8H- 7-oxa-1,3,6,10- tetraazacyclohepta[de]naphthalen- 5-yl)-5-chloro-4- cyclopropylphenyl (diastereomer 1) | 629.3 | (400 MHz, CD₃OD) δ 7.97-7.92 (m, 1H), 7.78-7.70 (m, 1H), 6.94- 6.90 (m, 1H), 6.80-6.75 (m, 1H), 6.74-6.71 (m, 1H), 6.65-6.57 (m, 1H), 5.31-5.06 (m, 1H), 4.53-4.26 (m, 2H), 3.80-3.67 (m, 1H), 3.60- 3.43 (m, 2H), 3.21-3.10 (m, 1H), 2.75-2.58 (m, 1H), 2.41-2.21 (m, 1H), 2.13-1.95 (m, 1H), 1.89-1.79 (m, 1H), 1.69-1.60 (m, 3H), 0.76- 0.58 (m, 2H), 0.25-0.02 (m, 2H). | It was obtained by resolving the compound prepared from I-A3, I-B22 and I-C3 by following the preparation steps of the compound 2; chiral HPLC resolution conditions: column: IG (2 × 25 cm); mobile phase: ethanol (0.1% ammonia water). |
| | 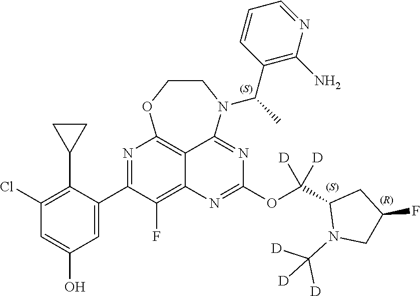 142 & 143 | | | | |
| 143 | | 3-(10-((R or S)-1-(2- | 629.3 | (400 MHz, CD₃OD) δ 7.98-7.91 | |
| | | aminopyridin-3-yl)ethyl)-4- | | (m, 1H), 7.79-7.71 (m, 1H), 6.94- | |
| | | fluoro-2-(((2S,4)-4-fluoro-1- | | 6.89 (m, 1H), 6.80-6.75 (m, 1H), | |
| | | (methyl-d₃)pyrrolidin-2- | | 6.74-6.72 (m, 1H), 6.66-6.56 (m, | |
| | | yl)methoxy-d₂)-9,10-dihydro-8H- | | 1H), 5.31-5.05 (m, 1H), 4.52-4.24 | |
| | | 7-oxa-1,3,6,10- | | (m, 2H), 3.80-3.67 (m, 1H), 3.58- | |
| | | tetraazacyclohepta[de]naphthalen- | | 3.43 (m, 2H), 3.23-3.10 (m, 1H), | |
| | | 5-yl)-5-chloro-4- | | 2.77-2.57 (m, 1H), 2.40-2.23 (m, | |
| | | cyclopropylphenol (diastereomer 2) | | 1H), 2.13-1.92 (m, 1H), 1.89-1.77 | |
| | | | | (m, 1H), 1.69-1.57 (m, 3H), 0.75- | |
| | | | | 0.58 (m, 2H), 0.22-0.05 (m, 2H). | |
| 144 | 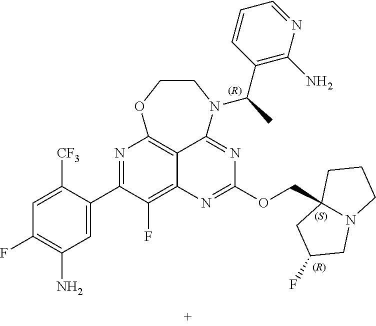 | 3-((R or S)-1-(5-(5-amino-4- fluoro-2- (trifluoromethyl)phenyl)-4- fluoro-2-(((2R,7aS)-2- fluorotetrahydro-1H-pyrrolizin- 7a(5H)-yl)methoxy)-8,9-dihydro- 10H-7-oxa-1,3,6,10- tetraazacyclohepta[de]naphthalen- 10-yl)ethyl)pyridin-2-amine (diastereomer 1) | 661.2 | (400 MHz, CD₃OD) δ 7.97-7.92 (m, 1H), 7.73 (d, J = 7.4 Hz, 1H), 7.35 (d, J = 11.8 Hz, 1H), 6.81 (d, J = 8.5 Hz, 1H), 6.78-6.73 (m, 1H), 6.62-6.54 (m, 1H), 5.43- 5.19 (m, 1H), 4.48-4.39 (m, 1H), 4.36-4.27 (m, 3H), 3.76-3.68 (m, 1H), 3.58-3.46 (m, 1H), 3.28-3.14 (m, 3H), 3.05-2.94 (m, 1H), 2.42- 2.11 (m, 3H), 2.03-1.86 (m, 3H), 1.67-1.60 (m, 3H). | It was obtained by resolving the compound 135; chiral HPLC resolution conditions: column: ODH (2 × 25 cm); mobile phase: n-heptane/ethanol (0.1% ammonia water) = 70:30. |
| | 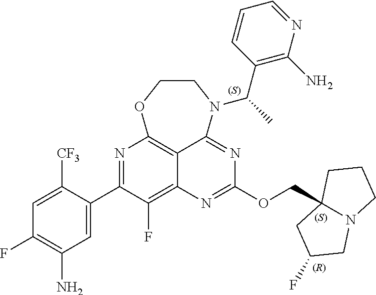 144 & 145 | | | | |
| 145 | | 3-((R or S)-1-(5-(5-amino-4- | 661.2 | (400 MHz, CD₃OD) δ 7.97-7.91 | |
| | | fluoro-2- | | (m, 1H), 7.74 (d, J = 7.5 Hz, 1H), | |
| | | (trifluoromethyl)phenyl)-4- | | 7.35 (d, J = 11.8 Hz, 1H), 6.81 (d, | |
| | | fluoro-2-(((2R,7aS)-2- | | J = 8.5 Hz, 1H), 6.79-6.73 (m, | |
| | | fluorotetrahydro-1H-pyrrolizin- | | 1H), 6.62-6.54 (m, 1H), 5.48-5.16 | |
| | | 7a(5H)-yl)methoxy)-8,9-dihydro- | | (m, 1H), 4.51-4.39 (m, 1H), 4.37- | |
| | | 10H-7-oxa-1,3,6,10- | | 4.24 (m, 3H), 3.78-3.69 (m, 1H), | |
| | | tetraazacyclohepta[de]naphthalen- | | 3.59-3.46 (m, 1H), 3.28-3.13 (m, | |
| | | 10-yl)ethyl)pyridin-2-amine | | 3H), 3.08-2.95 (m, 1H), 2.45-2.09 | |
| | | (diastereomer 2) | | (m, 3H), 2.02-1.83 (m, 3H), 1.63 | |
| | | | | (d, J = 6.9 Hz, 3H). | |
| 153 | 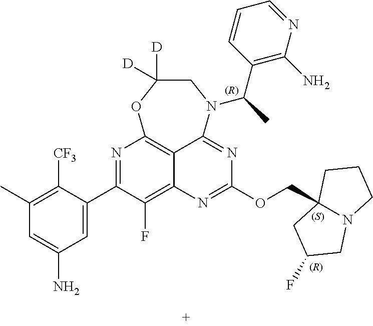 | 3-((R or S)-1-(5-(5-amino-3- methyl-2- (trifluoromethyl)phenyl)-4- fluoro-2-(((2R,7aS)-2- fluorotetrahydro-1H-pyrrolizin- 7a(5H)-yl)methoxy)-8,9-dihydro- 10H-7-oxa-1,3,6,10- tetraazacyclohepta[de]naphthalen- 10-yl-8,8-d₂)ethyl)pyridin-2- amine (diastereomer 1) | 659.3 | (400 MHz, CD₃OD) δ 7.94 (d, J = 3.7 Hz, 1H), 7.74 (d, J = 7.4 Hz, 1H), 6.80-6.73 (m, 1H), 6.66 (s, 1H), 6.62-6.55 (m, 1H), 6.38 (s, 1H), 5.30 (d, J = 53.5 Hz, 1H), 4.39-4.21 (m, 2H), 3.78-3.65 (m, 1H), 3.56-3.42 (m, 1H), 3.28- 3.16 (m, 3H), 3.07-2.98 (m, 1H), 2.44-2.38 (m, 3H), 2.35-2.12 (m, 3H), 2.05-1.90 (m, 3H), 1.63 (d, J = 6.9 Hz, 3H). | It was obtained by resolving the compound 140; chiral HPLC resolution conditions: column: ODH (2 × 25 cm); mobile phase: n-heptane/ ethanol (0.1% ammonia water) = 50:50. |
| | 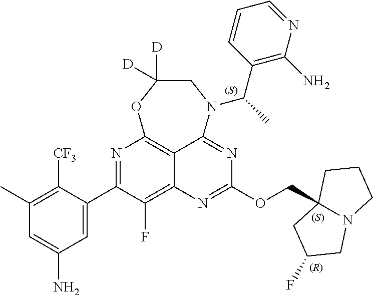 153 & 154 | | | | |
| 154 | | 3-((R or S)-1-(5-(5-amino-3- | 659.3 | (400 MHz, CD₃OD) δ 7.94 (d, J = | |
| | | methyl-2- | | 3.9 Hz, 1H), 7.74 (d, J = 7.5 Hz, | |
| | | (trifluoromethyl)phenyl)-4- | | 1H), 6.83-6.73 (m, 1H), 6.66 (s, | |
| | | fluoro-2-(((2R,7aS)-2- | | 1H), 6.63-6.53 (m, 1H), 6.38 (s, | |
| | | fluorotetrahydro-1H-pyrrolizin- | | 1H), 5.41-5.21 (m, 1H), 4.35-4.24 | |
| | | 7a(5H)-yl)methoxy)-8,9-dihydro- | | (m, 2H), 3.76-3.66 (m, 1H), 3.56- | |
| | | 10H-7-oxa-1,3,6,10- | | .45 (m, 1H), 3.27-3.13 (m, 3H), | |
| | | tetraazacyclohepta[de]naphthalen- | | 3.05-2.96 (m, 1H), 2.41-2.39 (m, | |
| | | 10-yl-8,8-d₂)ethyl)pyridin-2- | | 3H), 2.34-2.08 (m, 3H), 2.02-1.86 | |
| | | amine (diastereomer 2) | | (m, 3H), 1.63 (d, J = 6.9 Hz, 3H). | |
| 160 | 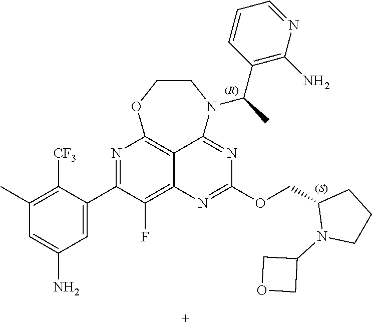 | 3-((R or S)-1-(5-(5-amino-3- methyl-2- (trifluoromethyl)phenyl)-4- fluoro-2-(((S)-1-(oxetan-3- yl)pyrrolidin-2-yl)methoxy)-8,9- dihydro-10H-7-oxa-1,3,6,10- tetraazacyclohepta[de]naphthalen- 10-yl)ethyl)pyridin-2-amine (diastereomer 1) | 655.2 | (400 MHz, CD₃OD) δ 7.94 (d, J = 5.0 Hz, 1H), 7.75 (d, J = 7.6 Hz, 1H), 6.81-6.74 (m, 1H), 6.66 (s, 1H), 6.63-6.55 (m, 1H), 6.38 (s, 1H), 4.84-4.78 (m, 1H), 4.76-4.66 (m, 3H), 4.48-4.34 (m, 3H), 4.34- 4.22 (m, 1H), 4.12-4.02 (m, 1H), 3.78-3.67 (m, 1H), 3.56-3.46 (m, 1H), 3.11-2.97 (m, 3H), 2.55-2.46 (m, 1H), 2.43-2.38 (m, 3H), 2.12- 2.01 (m, 1H), 1.91-1.74 (m, 3H), 1.63 (d, J = 6.9 Hz, 3H). | It was obtained by resolving the compound 123; chiral HPLC resolution conditions: column: ODH (2 × 25 cm); mobile phase: ethanol (0.1% ammonia water). |
| | 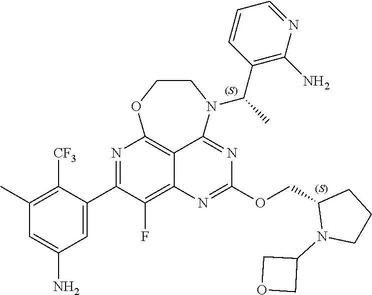 160 & 161 | | | | |
| 161 | | 3-((R or S)-1-(5-(5-amino-3- | 655.2 | (400 MHz, CD₃OD) δ 7.94 (d, | |
| | | methyl-2- | | J = 3.9 Hz, 1H), 7.74 (d, J = 7.2 Hz, | |
| | | (trifluoromethyl)phenyl)-4- | | 1H), 6.80-6.74 (m, 1H), 6.66 (s, | |
| | | fluoro-2-(((S)-1-(oxetan-3- | | 1H), 6.63-6.55 (m, 1H), 6.38 (s, | |
| | | yl)pyrrolidin-2-yl)methoxy)-8,9- | | 1H), 4.84-4.78 (m, 1H), 4.76-4.66 | |
| | | dihydro-10H-7-oxa-1,3,6,10- | | (m, 3H), 4.48-4.38 (m, 2H), 4.37- | |
| | | tetraazacyclohepta[de]naphthalen- | | 4.21 (m, 2H), 4.12-4.02 (m, 1H), | |
| | | 10-yl)ethyl)pyridin-2-amine | | 3.76-3.66 (m, 1H), 3.56-3.47 (m, | |
| | | (diastereomer 2) | | 1H), 3.11-2.99 (m, 2H), 2.55-2.45 | |
| | | | | (m, 1H), 2.43-2.38 (m, 3H), 2.11- | |
| | | | | 2.01 (m, 1H), 1.91-1.74 (m, 3H), | |
| | | | | 1.64 (d, J = 6.9 Hz, 3H). | |
| 166 | 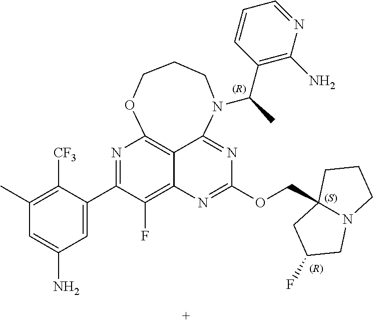 | 3-((R or S)-1-(5-(5-amino-3- methyl-2- (trifluoromethyl)phenyl)-4- fluoro-2-(((2R,7aS)-2- fluorotetrahydro-1H-pyrrolizin- 7a(5H)-yl)methoxy)-9,10- dihydro-77-oxa-1,3,6,11- tetraazacycloocta[de]naphthalen- 11(8H)-yl)ethyl)pyridin-2-amine (diastereomer 1) | 671.4 | (400 MHz, CD₃OD) δ 8.39-8.34 (m, 1H), 7.85-7.78 (m, 1H), 7.26- 7.20 (m, 1H), 6.70-6.63 (m, 1H), 6.50-6.43 (m, 1H), 5.35-5.18 (m, 1H), 4.74-4.65 (m, 1H), 4.41-4.19 (m, 3H), 3.98-3.87 (m, 1H), 3.45- 3.35 (m, 1H), 3.27-3.10 (m, 3H), 3.01-2.93 (m, 1H), 2.75-2.62 (m, 1H), 2.45-2.39 (m, 3H), 2.36-2.09 (m, 3H), 2.04-1.77 (m, 5H), 1.58- 1.51 (m, 3H). | It was obtained by resolving the compound prepared from I-A12, I-B1 and I-C10 by following the preparation steps of the compound 2; chiral HPLC resolution conditions: column: ODH (2 × 25 cm); mobile phase: n-heptane/ethanol (0.1% ammonia water) = 20:80. |
| | 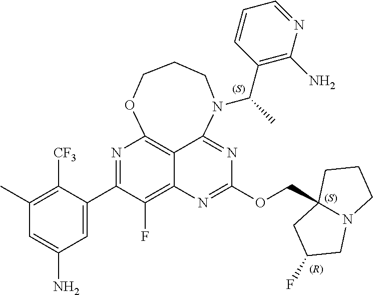 166 & 167 | | | | |
| 167 | | 3-((R or S)-1-(5-(5-amino-3- | 671.4 | (400 MHz, CD₃OD) δ 8.40-8.33 | |
| | | methyl-2- | | (m, 1H), 7.87-7.78 (m, 1H), 7.27- | |
| | | (trifluoromethyl)phenyl)-4- | | 7.20 (m, 1H), 6.70-6.64 (m, 1H), | |
| | | fluoro-2-(((2R,7aS)-2- | | 6.51-6.43 (m, 1H), 5.33-5.16 (m, | |
| | | fluorotetrahydro-1H-pyrrolizin- | | 1H), 4.76-4.65 (m, 1H), 4.45-4.23 | |
| | | 7a(5H)-yl)methoxy)-9,10- | | (m, 2H), 4.20-4.08 (m, 1H), 3.99- | |
| | | dihydro-7-oxa-1,3,6,11- | | 3.86 (m, 1H), 3.46-3.35 (m, 1H), | |
| | | tetraazacycloocta[de]naphthalen- | | 3.28-3.09 (m, 3H), 3.01-2.92 (m, | |
| | | 11(8H)-yl)ethyl)pyridin-2-amine | | 1H), 2.75-2.61 (m, 1H), 2.44-2.40 | |
| | | (diastereomer 2) | | (m, 3H), 2.37-2.05 (m, 3H), 2.04- | |
| | | | | 1.76 (m, 5H), 1.59-1.51 (m, 3H). | |
| 183 | 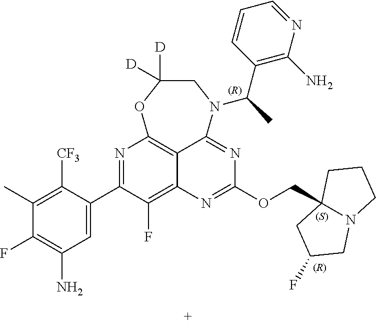 | 3-((R or S)-1-(5-(5-amino-4- fluoro-3-methyl-2- (trifluoromethyl)phenyl)-4- fluoro-2-(((2R,7aS)-2- fluorotetrahydro-1H-pyrrolizin- 7a(5H)-yl)methoxy)-8,9-dihydro- 10H-7-oxa-1,3,6,10- tetraazacyclohepta[de]naphthalen- 10-yl-8,8-d₂)ethyl)pyridin-2- amine (diastereomer 1) | 677.3 | (400 MHz, CD₃OD) δ 7.98-7.90 (m, 1H), 7.74 (d, J = 6.9 Hz, 1H), 6.82-6.74 (m, 1H), 6.63-6.54 (m, 2H), 5.41-5.22 (m, 1H), 4.41-4.27 (m, 2H), 3.77-3.68 (m, 1H), 3.54- 3.46 (m, 1H), 3.29-3.19 (m, 3H), 3.10-2.99 (m ,1H), 2.40-2.36 (m, 3H), 2.35-2.13 (m, 3H), 2.06-1.89 (m, 3H), 1.64 (d, J = 6.9 Hz, 3H). | It was obtained by resolving the compound 157; chiral HPLC resolution conditions: column: ODH (2 × 25 cm); mobile phase: n-heptane/ ethanol (0.1% ammonia water) = 20:80. |
| | 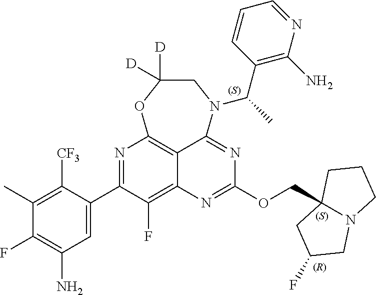 183 & 184 | | | | |
| 184 | | 3-((R or S)-1-(5-(5-amino-4- | 677.3 | (400 MHz, CD₃OD) δ 7.95 (d, J = | |
| | | fluoro-3-methyl-2- | | 3.7 Hz, 1H), 7.74 (d, J = 7.1 Hz, | |
| | | (trifluoromethyl)phenyl)-4- | | 1H), 6.82-6.73 (m, 1H), 6.63-6.53 | |
| | | fluoro-2-(((2R,7aS)-2- | | (m, 2H), 5.41-5.18 (m, 1H), 4.42- | |
| | | fluorotetrahydro-1H-pyrrolizin- | | 4.25 (m, 2H), 3.78-3.68 (m, 1H), | |
| | | 7a(5H)-yl)methoxy)-8,9-dihydro- | | 3.55-3.48 (m, 1H), 3.29-3.18 (m, | |
| | | 10H-7-oxa-1,3,6,10- | | 3H), 3.06-2.99 (m, 1H), 2.43-2.36 | |
| | | tetraazacyclohepta[de]naphthalen- | | (m, 3H), 2.35-2.09 (m, 3H), 2.05- | |
| | | 10-yl-8,8-d₂)ethyl)pyridin-2- | | 1.88 (m, 3H), 1.64 (d, J = 6.9 Hz, | |
| | | amine (diastereomer 2) | | 3H). | |
| 199 | 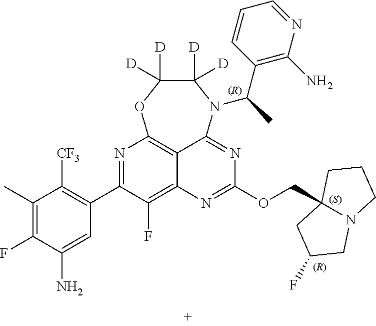 | 3-((R or S)-1-(5-(5-amino-4- fluoro-3-methyl-2- (trifluoromethyl)phenyl)-4- fluoro-2-(((2R,7aS)-2- fluorotetrahydro-1H-pyrrolizin- 7a(5H)-yl)methoxy)-8,9-dihydro- 10H-7-oxa-1,3,6,10- tetraazacyclohepta[de]naphthalen- 10-yl-8,8,9,9-d₄)ethyl)pyridin-2- amine (diastereomer 1) | 679.4 | (400 MHz, CD₃OD) δ 7.98-7.91 (m, 1H), 7.79-7.69 (m, 1H), 6.80- 6.73 (m, 1H), 6.63-6.53 (m, 2H), 5.40-5.19 (m, 1H), 4.32-4.25 (m, 2H), 3.27-3.13 (m, 3H), 3.05-2.96 (m, 1H), 2.41-2.35 (m, 3H), 2.34- 2.11 (m, 3H), 2.03-1.88 (m, 3H), 1.63 (d, J = 7.0 Hz, 3H). | It was obtained by resolving the compound prepared from I-A18, I-B1 and I-C17 by following the preparation steps of the compound 2; chiral HPLC resolution conditions: column: ODH (2 × 25 cm); mobile phase: n-heptane/ ethanol (0.1% ammonia water) = 20:80. |
| | 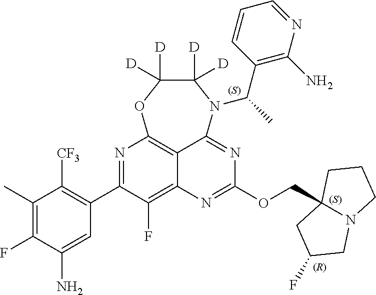 199 & 200 | | | | |
| 200 | | 3-((R or S)-1-(5-(5-amino-4- | 679.4 | (400 MHz, CD₃OD) δ 7.98-7.92 | |
| | | fluoro-3-methyl-2- | | (m, 1H), 7.78-7.71 (m, 1H), 6.80- | |
| | | (trifluoromethyl)phenyl)-4- | | 6.73 (m, 1H), 6.62-6.53 (m, 2H), | |
| | | fluoro-2-(((2R,7aS)-2- | | 5.44-5.22 (m, 1H), 4.41-4.26 (m, | |
| | | fluorotetrahydro-1H-pyrrolizin- | | 2H), 3.29-3.21 (m, 3H), 3.10-2.99 | |
| | | 7a(5H)-yl)methoxy)-8,9-dihydro- | | (m, 1H), 2.42-2.36 (m, 3H), 2.35- | |
| | | 10H-7-oxa-1,3,6,10- | | 2.10 (m, 3H), 2.04-1.86 (m, 3H), | |
| | | tetraazacyclohepta[de]naphthalen- | | 1.63 (d, J = 6.9 Hz, 3H). | |
| | | 10-yl-8,8,9,9-d₄)ethyl)pyridin-2- | | | |
| | | amine (diastereomer 2) | | | |
| 202 | 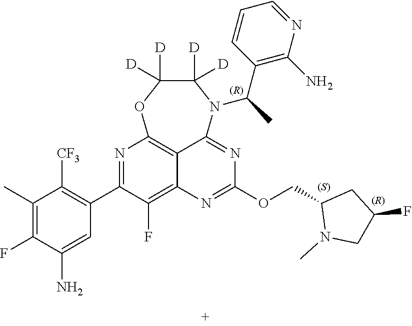 | 3-((R or S)-1-(5-(5-amino-4- fluoro-3-methyl-2- (trifluoromethyl)phenyl)-4- fluoro-2-(((2S,4R)-4-fluoro-1- methylpyrrolidin-2-yl)methoxy)- 8,9-dihydro-10H-7-oxa-1,3,6,10- tetraazacyclohepta[de]naphthalen- 10-yl-8,8,9,9-d₄)ethyl)pyridin-2- amine (diastereomer 1) | 653.3 | (400 MHz, CD₃OD) δ 7.98-7.92 (m, 1H), 7.78-7.72 (m, 1H), 6.81- 6.74 (m, 1H), 6.64-6.55 (m, 2H), 5.29-5.08 (m, 1H), 4.65-4.46 (m, 2H), 3.60-3.45 (m, 1H), 3.25-3.14 (m, 1H), 2.79-2.62 (m, 1H), 2.57 (s, 3H), 2.41-2.36 (m, 3H), 2.34- 2.23 (m, 1H), 2.21-1.96 (m, 1H), 1.64 (d, J = 6.9 Hz, 3H). | It was obtained by resolving the compound prepared from I-A18, I-B10 and I-C17 by following the preparation steps of the compound 2; chiral HPLC resolution conditions: column: IG (2 × 25 cm); mobile phase: n-heptane/ ethanol (0.1% ammonia water) = 20:80. |
| | 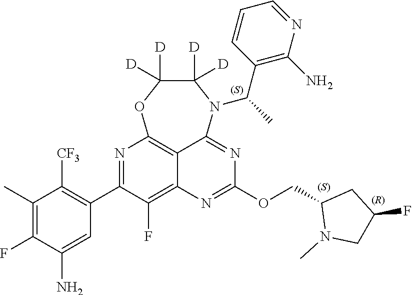 202 & 203 | | | | |
| 203 | | 3-((R or S)-1-(5-(5-amino-4- | 653.3 | (400 MHz, CD₃OD) δ 7.98-7.92 | |
| | | fluoro-3-methyl-2- | | (m, 1H), 7.77-7.73 (m, 1H), 6.81- | |
| | | (trifluoromethyl)phenyl)-4- | | 6.74 (m, 1H), 6.64-6.55 (m, 2H), | |
| | | fluoro-2-(((2S,4R)-4-fluoro-1- | | 5.28-5.07 (m, 1H), 4.60-4.47 (m, | |
| | | methylpyrrolidin-2-yl)methoxy)- | | 2H), 3.56-3.44 (m, 1H), 3.19-3.11 | |
| | | 8,9-dihydro-10H-7-oxa-1,3,6,10- | | (m, 1H), 2.74-2.60 (m, 1H), 2.55 | |
| | | tetraazacyclohepta[de]naphthalen- | | (s, 3H), 2.41-2.36 (m, 3H), 2.34- | |
| | | 10-yl-8,8,9,9-d₄)ethyl)pyridin-2- | | 2.23 (m, 1H), 2.12-1.99 (m, 1H), | |
| | | amine (diastereomer 2) | | 1.64 (d, J = 6.9 Hz, 3H). | |
| 206 | 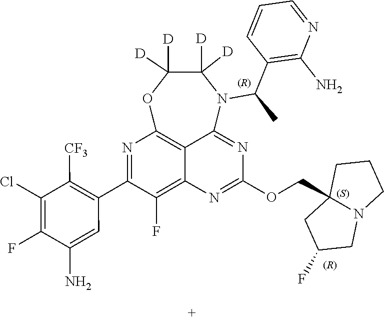 | 3-((R or S)-1-(5-(5-amino-3- chloro-4-fluoro-2- (trifluoromethyl)phenyl)-4- fluoro-2-(((2R,7aS)-2- fluorotetrahydro-1H-pyrrolizin- 7a(5H)-yl)methoxy)-8,9-dihydro- 10H-7-oxa-1,3,6,10- tetraazacyclohepta[de]naphthalen- 10-yl-8,8,9,9-d_{₄})ethyl)pyridin-2- amine (diastereomer 1) | 699.3 | (400 MHz, CD₃OD) δ 7.98-7.91 (m, 1H), 7.78-7.70 (m, 1H), 6.81- 6.74 (m, 1H), 6.70-6.65 (m, 1H), 6.63-6.54 (m, 1H), 5.38-5.21 (m, 1H), 4.34-4.26 (m, 2H), 3.27-3.15 (m, 3H), 3.06-2.96 (m, 1H), 2.39- 2.12 (m, 3H), 2.05-1.89 (m, 3H), 1.63 (d, J = 6.9 Hz, 3H). | It was obtained by resolving the compound prepared from I-A28, I-B1 and I-C17 by following the preparation steps of the compound 2; chiral HPLC resolution conditions: column: ODH (2 × 25 cm); mobile phase: n-heptane/ ethanol (0.1% ammonia water) = 50:50. |
| | 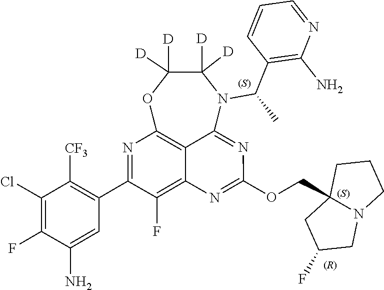 206 & 207 | | | | |
| 207 | | 3-((R or S)-1-(5-(5-amino-3- | 699.3 | (400 MHz, CD₃OD) δ 8.00-7.92 | |
| | | chloro-4-fluoro-2- | | (m, 1H), 7.80-7.71 (m, 1H), 6.80- | |
| | | (trifluoromethyl)phenyl)-4- | | 6.73 (m, 1H), 6.71-6.65 (m, 1H), | |
| | | fluoro-2-(((2R,7aS)-2- | | 6.62-6.54 (m, 1H), 5.42-5.18 (m, | |
| | | fluorotetrahydro-1H-pyrrolizin- | | 1H), 4.44-4.19 (m, 2H), 3.29-3.15 | |
| | | 7a(5H)-yl)methoxy)-8,9-dihydro- | | (m, 3H), 3.06-2.92 (m, 1H), 2.42- | |
| | | 10H-7-oxa-1,3,6,10- | | 2.09 (m, 3H), 2.03-1.85 (m, 3H), | |
| | | tetraazacyclohepta[de]naphthalen- | | 1.64 (d, J = 6.9 Hz, 3H). | |
| | | 10-yl-8,8,9,9-d₄)ethyl)pyridin-2- | | | |
| | | amine (diastereomer 2) | | | |
| 208 | 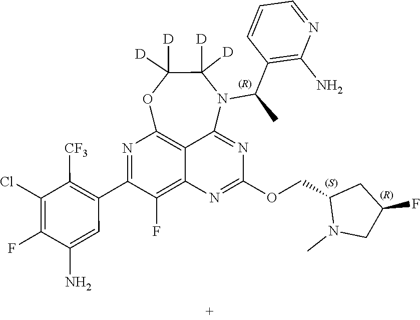 | 3-((R or S)-1-(5-(5-amino-3- chloro-4-fluoro-2- (trifluoromethyl)phenyl)-4- fluoro-2-(((2S,4R)-4-fluoro-1- methylpyrrolidin-2-yl)methoxy)- 8,9-dihydro-10H-7-oxa-1,3,6,10- tetraazacyclohepta[de]naphthalen- 10-yl-8,8,9,9-d₄)ethyl)pyridin-2- amine (diastereomer 1) | 673.2 | (400 MHz, CD₃OD) δ 8.00-7.91 (m, 1H), 7.80-7.71 (m, 1H), 6.85- 6.74 (m, 1H), 6.70-6.64 (m, 1H), 6.64-6.55 (m, 1H), 5.28-5.08 (m, 1H), 4.63-4.48 (m, 2H), 3.60-3.43 (m, 1H), 3.19-3.11 (m, 1H), 2.73- 2.59 (m, 1H), 2.55 (s, 3H), 2.39- 2.23 (m, 1H), 2.12-1.95 (m, 1H), 1.64 (d, J = 6.9 Hz, 3H). | It was obtained by resolving the compound prepared from I-A28, I-B10 and C-17 by following the preparation steps of the compound 2; chiral HPLC resolution conditions: column: ODH (2 × 25 cm); mobile phase: n-heptane/ ethanol (0.1% ammonia water) = 50.50. |
| | 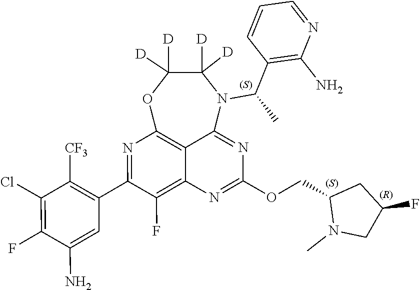 208 & 209 | | | | |
| 209 | | 3-((R or S)-1-(5-(5-amino-3- | 673.2 | (400 MHz, CD₃OD) δ 7.98-7.92 | |
| | | chloro-4-fluoro-2- | | (m, 1H), 7.79-7.70 (m, 1H), 6.81- | |
| | | (trifluoromethyl)phenyl)-4- | | 6.73 (m, 1H), 6.70-6.65 (m, 1H), | |
| | | fluoro-2-(((2S,4R)-4-fluoro-1- | | 6.63-6.56 (m, 1H), 5.30-5.09 (m, | |
| | | methylpyrrolidin-2-yl)methoxy)- | | 1H), 4.62-4.50 (m, 2H), 3.58-3.43 | |
| | | 8,9-dihydro-10H-7-oxa-1,3,6,10- | | (m, 1H), 3.20-3.09 (m, 1H), 2.60 | |
| | | tetraazacyclohepta[de]naphthalen- | | (s, 1H), 2.55 (s, 3H), 2.36-2.21 | |
| | | 10-yl-8,8,9,9-d₄)ethyl)pyridin-2- | | (m, 1H), 2.12-1.96 (m, 1H), 1.64 | |
| | | amine (diastereomer 2) | | (d, J = 6.9 Hz, 3H). | |
| 210 | 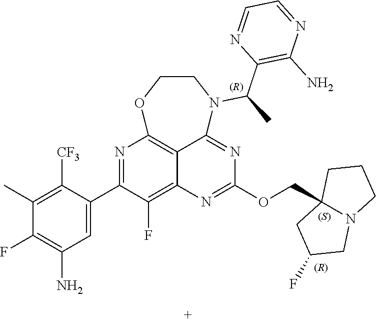 | 3-((R or S)-1-(5-(5-amino-4- fluoro-3-methyl-2- (trifluoromethyl)phenyl)-4- fluoro-2-(((2R,7aS)-2- fluorotetrahydro-1H-pyrrolizin- 7a(5H)-yl)methoxy)-8,9-dihydro- 10H-7-oxa-1,3,6,10- tetraazacyclohepta[de]naphthalen- 10-yl)ethyl)pyrazin-2-amine (diastereomer 1) | 676.3 | (400 MHz, CD₃OD) δ 7.92-7.88 (m, 1H), 7.85-7.79 (m, 1H), 6.65- 6.45 (m, 2H), 5.39-5.15 (m, 1H), 4.63-4.37 (m, 2H), 4.32-4.19 (m, 2H), 4.04-3.76 (m, 2H), 3.27-3.14 (m, 3H), 3.07-2.93 (m, 1H), 2.39- 2.34 (m, 3H), 2.32-2.09 (m, 3H), 2.04-1.84 (m, 3H), 1.73-1.60 (m, 3H). | It was obtained by resolving the compound prepared from I-A18, I-B1 and I-C18 by following the preparation steps of the compond 2; chiral HPLC resolution conditions: column: ODH (2 × 25 cm); mobile phase: n-heptane/ethanol (0.1% ammonia water) = 50:50. |
| | 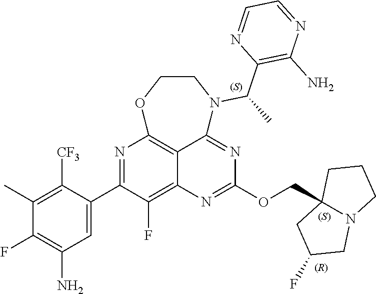 210 & 211 | | | | |
| 211 | | 3-((R or S)-1-(5-(5-amino-4- | 676.3 | (400 MHz, CD₃OD) δ 7.92-7.87 | |
| | | fluoro-3-methyl-2- | | (m, 1H), 7.85-7.79 (m, 1H), 6.68- | |
| | | (trifluoromethyl)phenyl)-4- | | 6.43 (m, 2H), 5.43-5.16 (m, 1H), | |
| | | fluoro-2-(((2R,7aS)-2- | | 4.62-4.38 (m, 2H), 4.34-4.15 (m, | |
| | | fluorotetrahydro-1H-pyrrolizin- | | 2H), 4.00-3.74 (m, 2H), 3.27-3.11 | |
| | | 7a(5H)-yl)methoxy)-8,9-dihydro- | | (m, 3H), 3.05-2.94 (m, 1H), 2.39- | |
| | | 10H-7-oxa-1,3,6,10- | | 2.33 (m, 3H), 2.32-2.08 (m, 3H), | |
| | | tetraazacyclohepta[de]naphthalen- | | 2.04-1.84 (m, 3H), 1.72-1.60 (m, | |
| | | 10-yl)ethyl)pyrazin-2-amine | | 3H). | |
| | | (diastereomer 2) | | | |
| 212 | 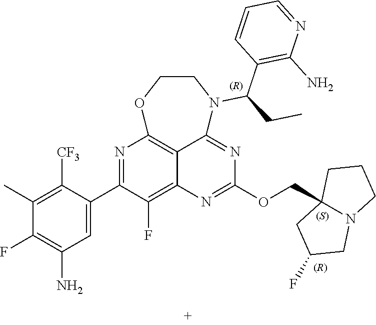 | 3-((R or S)-1-(5-(5-amino-4- fluoro-3-methyl-2- (trifluoromethyl)phenyl)-4- fluoro-2-(((2R,7aS)-2- fluorotetrahydro-1H-pyrrolizin- 7a(5H)-yl)methoxy)-8,9-dihydro- 10H-7-oxa-1,3,6,10- tetraazacyclohepta[de]naphthalen- 10-yl)propyl)pyridin-2-amine (diastereomer 1) | 689.3 | (400 MHz, CD₃OD) δ 7.94 (d, J = 5.0 Hz, 1H), 7.71 (d, J = 7.6 Hz, 1H), 6.77-6.73 (m, 1H), 6.58 (d, J = 8.5 Hz, 1H), 6.49-6.34 (m, 1H), 5.42-5.17 (m, 1H), 4.54-4.12 (m, 4H), 3.79-3.49 (m, 2H), 3.40- 3.19 (m, 3H), 3.10-2.98 (m, 1H), 2.45-1.86 (m, 11H), 0.98 (t, J = 7.1 Hz, 3H). | It was obtained by resolving the compound prepared from I-A18, I-B1 and I-C19 by following the preparation steps of the compound 2; chiral HPLC resolution conditions: column: ODH (2 × 25 cm); mobile phase: n-heptane/ethanol (0.1% ammonia water) = 50:50. |
| | 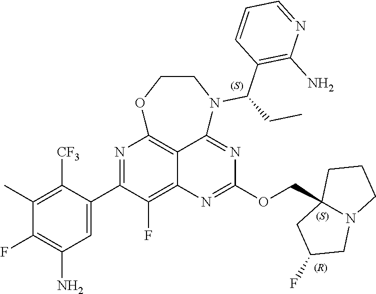 212 & 213 | | | | |
| 213 | | 3-((R or S)-1-(5-(5-amino-4- | 689.3 | (400 MHz, CD₃OD) δ 7.94 (d, J = | |
| | | fluoro-3-methyl-2- | | 5.0 Hz, 1H), 7.71 (d, J = 7.0 Hz, | |
| | | (trifluoromethyl)phenyl)-4- | | 1H), 6.79-6.75 (m, 1H), 6.58 (d, J = | |
| | | fluoro-2-(((2R,7aS)-2- | | 8.4 Hz, 1H), 6.45-6.40 (m, 1H), | |
| | | fluorotetrahydro-1H-pyrrolizin- | | 5.45-5.20 (m, 1H), 4.52-4.11 (m, | |
| | | 7a(5H)-yl)methoxy)-8,9-dihydro- | | 4H), 3.80-3.49 (m, 2H), 3.43- | |
| | | 10H-7-oxa-1,3,6,10- | | 3.19 (m, 3H), 3.10-3.02 (m, 1H), | |
| | | tetraazacyclohepta[de]napthalen- | | 2.48-1.83 (m, 11H), 0.96 (t, J = | |
| | | 10-yl)propyl)pyridin-2-amine | | 6.2, 3H). | |
| | | (diastereomer 2) | | | |
| 214 | 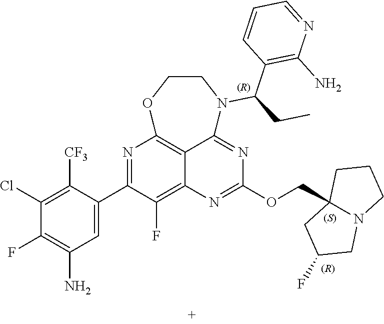 | 3-((R or S)-1-(5-(5-amino-3- chloro-4-fluoro-2- (trifluoromethyl)phenyl)-4- fluoro-2-(((2R,7aS)-2- fluorotetrahydro-1H-pyrrolizin- 7a(5H)-yl)methoxy)-8,9-dihydro- 10H-7-oxa-1,3,6,10- tetraazacyclohepta[de]naphthalen- 10-yl)propyl)pyridin-2-amine (diastereomer 1) | 709.2 | (400 MHz, CD₃OD) δ 7.96-7.92 (m, 1H), 7.72 (d, J = 6.5 Hz, 1H), 6.78-7.74 (m, 1H), 6.67 (d, J = 7.9 Hz, 1H), 6.48-6.37 (m, 1H), 5.40-5.20 (m, 1H), 4.51-4.14 (m, 4H), 3.77-3.50 (m, 2H), 3.28-3.15 (m, 3H), 3.05-2.97 (m, 1H), 2.41- 1.90 (m, 8H), 0.98 (t, J = 7.2 Hz, 3H). | It was obtained by resolving the compound prepared from I-A28, I-B1 and I-C19 by following the preparation steps of the compound 2; chiral HPLC resolution conditions: column: ODH (2 × 25 cm); mobile phase: n-heptane/ethanol (0.1% ammonia water) = 70:30. |
| | 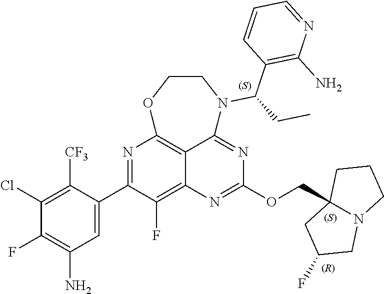 214 & 215 | | | | |
| 215 | | 3-((R or S)-1-(5-(5-amino-3- | 709.2 | (400 MHz, CD₃OD) δ 7.96-7.92 | |
| | | chloro-4-fluoro-2- | | (m, 1H), 7.72 (d, J = 8.0 Hz, 1H), | |
| | | (trifluoromethyl)phenyl)-4- | | 6.78-6.74 (m, 1H), 6.67 (d, J = | |
| | | fluoro-2-(((2R,7aS)-2- | | 7.3 Hz, 1H), 6.48-6.37 (m, 1H), | |
| | | fluorotetrahydro-1H-pyrrolizin- | | 5.43-5.17 (m, 1H), 4.57-4.10 (m, | |
| | | 7a(5H)-yl)methoxy)-8,9-dihydro- | | 4H), 3.77-3.50 (m, 2H), 3.29-3.15 | |
| | | 10H-7-oxa-1,3,6,10- | | (m, 3H), 3.04-2.97 (m, 1H), 2.43- | |
| | | tetraazacyclohepta[de]naphthalen- | | 1.79 (m, 8H), 0.97 (t, J = 6.2 Hz, | |
| | | 10-yl)propyl)pyridin-2-amine | | 3H). | |
| | | (diastereomer 2) | | | |
| 216 | 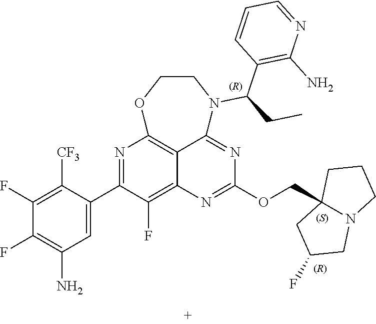 | 3-((R or S)-1-(5-(5-amino-3,4- difluoro-2- (trifluoromethyl)phenyl)-4- fluoro-2-(((2R,7aS)-2- fluorotetrahydro-1H-pyrrolizin- 7a(5H)-yl)methoxy)-8,9-dihydro- 10H-7-oxa-1,3,6,10- tetraazacyclohepta[de]naphthalen- 10-yl)propyl)pyridin-2-amine (diastereomer 1) | 693.3 | (400 MHz, CD₃OD) δ 7.98-7.91 (m, 1H), 7.76-7.68 (m, 1H), 6.80- 6.71 (m, 1H), 6.62-6.55 (m, 1H), 6.48-6.38 (m, 1H), 5.41-5.19 (m, 1H), 4.51-4.17 (m, 4H), 3.78- 3.56 (m, 2H), 3.27-3.17 (m, 2H), 3.07-2.95 (m, 1H), 2.46-1.76 (m, 9H), 1.04-0.95 (m, 3H). | It was obtained by resolving the compound prepared from I-A29, I-B1 and I-C19 by following the preparation steps of the compound 2; chiral HPLC resolution conditions: column: ODH (2 × 25 cm); mobile phase: n-heptane/ethanol (0.1% ammonia water) = 70:30. |
| | 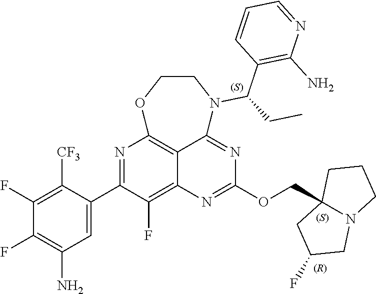 216 & 217 | | | | |
| 217 | | 3-((R or S)-1-(5-(5-amino-3,4- | 693.3 | (400 MHz, CD₃OD) δ 7.98-7.90 | |
| | | difluoro-2- | | (m, 1H), 7.79-7.68 (m, 1H), 6.83- | |
| | | (trifluoromethyl)phenyl)-4- | | 6.71 (m, 1H), 6.61-6.53 (m, 1H), | |
| | | fluoro-2-(((2R,7aS)-2- | | 6.49-6.37 (m, 1H), 5.46-5.20 (m, | |
| | | fluorotetrahydro-1H-pyrrolizin- | | 1H), 4.51-4.21 (m, 4H), 3.80-3.52 | |
| | | 7a(5H)-yl)methoxy)-8,9-dihydro- | | (m, 2H), 3.26-3.18 (m, 2H), 3.07- | |
| | | 10H-7-oxa-1,3,6,10- | | 2.95 (m, 1H), 2.40-1.88 (m, 9H), | |
| | | tetraazacyclohepta[de]naphthalen- | | 1.02-0.95 (m, 3H). | |
| | | 10-yl)propyl)pyridin-2-amine | | | |
| | | (diastereomer 2) | | | |
| 219 | 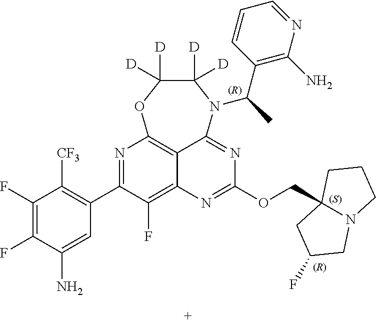 | 3-((R or S)-1-(5-(5-amino-3,4- difluoro-2- (trifluoromethyl)phenyl)-4- fluoro-2-(((2R,7aS)-2- fluorotetrahydro-1H-pyrrolizin- 7a(5H)-yl)methoxy)-8,9-dihydro- 10H-7-oxa-1,3,6,10- tetraazacyclohepta[de]naphthalen- 10-yl-8,8,9,9-d₄)ethyl)pyridin-2- amine (diastereomer 1) | 683.2 | (400 MHz, CD₃OD) δ 7.99-7.89 (m, 1H), 7.78-7.72 (m, 1H), 6.81- 6.73 (m, 1H), 6.64-6.52 (m, 2H), 5.42-5.20 (m, 1H), 4.36-4.21 (m, 2H), 3.29-3.09 (m, 3H), 3.05-2.95 (m, 1H), 2.38-2.06 (m, 3H), 2.05- 1.82 (m, 3H), 1.68-1.59 (m, 3H). | It was obtained by resolving the compound prepared from I-A29, I-B1 and I-C17 by following the preparation steps of the compound 2; chiral HPLC resolution conditions: column: ODH (2 × 25 cm); mobile phase: n-heptane/ethanol (0.1% ammonia water) = 70:30. |
| | 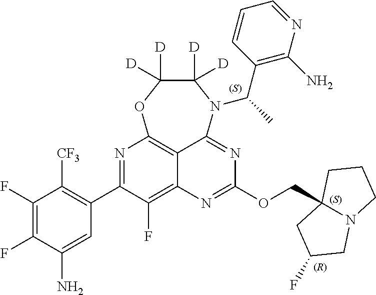 219 & 220 | | | | |
| 220 | | 3-((R or S)-1-(5-(5-amino-3,4- | 683.2 | (400 MHz, CD₃OD) δ 8.02-7.89 | |
| | | difluoro-2- | | (m, 1H), 7.79-7.72 (m, 1H), 6.82- | |
| | | (trifluoromethyl)phenyl)-4- | | 6.71 (m, 1H), 6.64-6.51 (m, 2H), | |
| | | fluoro-2-(((2R,7aS)-2- | | 5.39-5.22 (m, 1H), 4.35-4.26 (m, | |
| | | fluorotetrahydro-1H-pyrrolizin- | | 2H), 3.28-3.11 (m, 3H), 3.06-2.94 | |
| | | 7a(5H)-yl)methoxy)-8,9-dihydro- | | (m, 1H), 2.40-2.10 (m, 3H), 2.03- | |
| | | 10H-7-oxa-1,3,6,10- | | 1.83 (m, 3H), 1.69-1.57 (m, 3H). | |
| | | tetraazacyclohepta[de]naphthalen- | | | |
| | | 10-yl-8,8,9,9-d₄)ethyl)pyridin-2- | | | |
| | | amine (diastereomer 2) | | | |
| 222 | 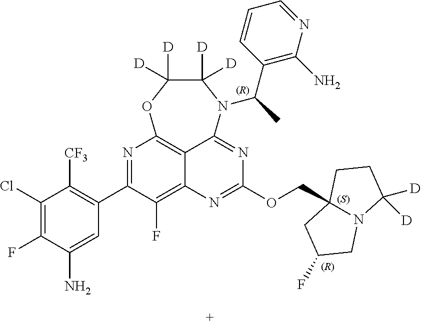 | 3-((R or S)-1-(5-(5-amino-3- chloro-4-fluoro-2- (trifluoromethyl)phenyl)-4- fluoro-2-(((2R,7aS)-2- fluorotetrahydro-1H-pyrrolizin- 7a(5H)-yl-5,5-d₂)methoxy)-8,9- dihydro-10H-7-oxa-1,3,6,10- tetraazacyclohepta[de]naphthalen- 10-yl-8,8,9,9-d₄)ethyl)pyridin-2- amine (diastereomer 1) | 701.2 | (400 MHz, CD₃OD) δ 7.98-7.90 (m, 1H), 7.78-7.68 (m, 1H), 6.81- 6.72 (m, 1H), 6.72-6.64 (m, 1H), 6.63-6.54 (m, 1H), 5.42-5.20 (m, 1H), 4.35-4.24 (m, 2H), 3.28-3.11 (m, 2H), 2.40-2.10 (m, 3H), 2.06- 1.84 (m, 3H), 1.63 (d, J = 6.9 Hz, 3H). | It was obtained by resolving the compound prepared from I-A28, I-B27 and I-C17 by following the preparation steps of the compound 2; chiral HPLC resolution conditions: column: ODH (2 × 25 cm); mobile phase: n-heptane/ethanol (0.1% ammonia water) = 50:50. |
| | 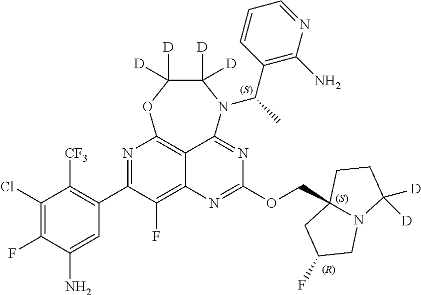 222 & 223 | | | | |
| 223 | | 3-((R or S)-1-(5-(5-amino-3- | 701.4 | (400 MHz, CD₃OD) δ 7.97-7.89 | |
| | | chloro-4-fluoro-2- | | (m, 1H), 7.78-7.67 (m, 1H), 6.80- | |
| | | (trifluoromethyl)phenyl)-4- | | 6.72 (m, 1H), 6.71-6.64 (m, 1H), | |
| | | fluoro-2-(((2R,7aS)-2- | | 6.62-6.52 (m, 1H), 5.46-5.17 (m, | |
| | | fluorotetrahydro-1H-pyrrolizin- | | 1H), 4.41-4.22 (m, 2H), 3.28-3.11 | |
| | | 7a(5H)-yl-5,5-d₂)methoxy)-8,9- | | (m, 2H), 2.40-2.08 (m, 3H), 2.01- | |
| | | dihydro-10H-7-oxa-1,3,6,10- | | 1.83 (m, 3H), 1.63 (d, J = 6.9 Hz, | |
| | | tetraazacyclohepta[de]naphthalen- | | 3H). | |
| | | 10-yl-8,8,9,9-d₄)ethyl)pyridin-2- | | | |
| | | amine (diastereomer 2) | | | |
| 247 | 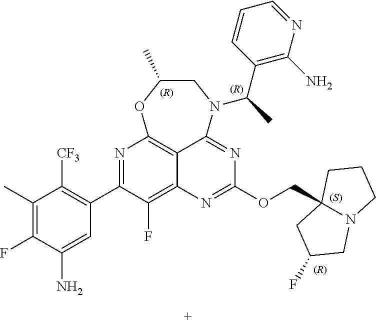 | 3-((R or S)-1-((R)-5-(5-amino-4- fluoro-3-methyl-2- (trifluoromethyl)phenyl)-4- fluoro-2-(((2R,7aS)-2- fluorotetrahydro-1H-pyrrolizin- 7a(5H)-yl)methoxy)-8-methyl- 8,9-dihydro-10H-7-oxa-1,3,6,10- tetraazacyclohepta[de]naphthalen- 10-yl)ethyl)pyridin-2-amine (diastereomer 1) | 689.3 | (400 MHz, CD₃OD) δ 8.02-7.94 (m, 1H), 7.80-7.72 (m, 1H), 6.84- 6.74 (m, 1H), 6.70-6.45 (m, 2H), 5.39-5.21 (m, 1H), 4.39-4.17 (m, 3H), 3.57-3.40 (m, 2H), 3.29-3.11 (m, 3H), 3.05-2.96 (m, 1H), 2.40- 2.10 (m, 6H), 2.03-1.84 (m, 3H), 1.64-1.55 (m, 3H), 1.18-1.14 (m, 3H). | It was obtained by resolving the compound prepared from I-A18, I-B1 and I-C20 by following the preparation steps of the compound 2; chiral HPLC resolution conditions: column: ODH (2 × 25 cm); mobile phase: n-heptane/ethanol (0.1% ammonia water) = 70:30. |
| | 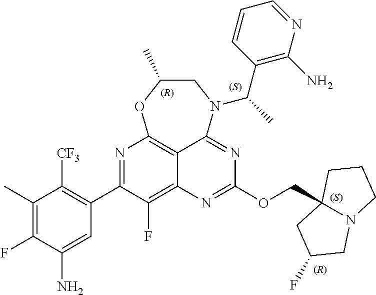 247 & 248 | | | | |
| 248 | | 3-((R or S)-1-((R)-5-(5-amino-4- | 689.3 | (400 MHz, CD₃OD) δ 7.99-7.86 | |
| | | fluoro-3-methyl-2- | | (m, 1H), 7.79-7.71 (m, 1H), 6.81- | |
| | | (trifluoromethyl)phenyl)-4- | | 6.71 (m, 1H), 6.69-6.50 (m, 2H), | |
| | | fluoro-2-(((2R,7aS)-2- | | 5.38-5.21 (m, 1H), 4.66-4.55 (m, | |
| | | fluorotetrahydro-1H-pyrrolizin- | | 1H), 4.35-4.25 (m, 2H), 3.70-3.57 | |
| | | 7a(5H)-yl)methoxy)-8-methyl- | | (m, 1H), 3.28-3.12 (m, 3H), 3.05- | |
| | | 8,9-dihydro-10H-7-oxa-1,3,6,10- | | 2.95 (m, 1H), 2.39-2.10 (m, 6H), | |
| | | tetraazacyclohepta[de]naphthalen- | | 2.03-1.85 (m, 3H), 1.70-1.59 (m, | |
| | | 10-yl)ethyl)pyridin-2-amine | | 3H), 1.24-1.11 (m, 3H). | |
| | | (diastereomer 2) | | | |
| 250 | 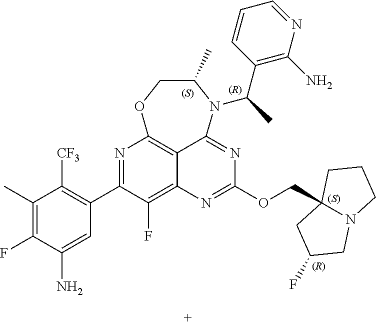 | 3-((R or S)-1-((S)-5-(5-amino-4- fluoro-3-methyl-2- (trifluoromethyl)phenyl)-4- fluoro-2-(((2R,7aS)-2- fluorotetrahydro-1H-pyrrolizin- 7a(5H)-yl)methoxy)-9-methyl- 8,9-dihydro-10H-7-oxa-1,3,6,10- tetraazacyclohepta[de]naphthalen- 10-yl)ethyl)pyridin-2-amine (diastereomer 1) | 689.3 | (400 MHz, CD₃OD) δ 7.95 (d, J = 1.6 Hz, 1H), 7.78 (d, J = 7.4 Hz, 1H), 6.80-6.69 (m, 2H), 6.69-6.49 (m, 1H), 5.40-5.19 (m, 1H), 4.64- 4.55 (m, 1H), 4.37-4.22 (m, 3H), 4.10-3.98 (m, 1H), 3.27-3.10 (m, 3H), 3.05-2.95 (m, 1H), 2.42-2.09 (m, 6H), 2.05-1.81 (m, 3H), 1.65- (d, J = 6.8 Hz, 3H), 0.66 (d, J = 6.8 Hz, 3H). | It was obtained by resolving the compound prepared from I-A18, I-B1 and I-C21 by following the preparation steps of the compound 2; chiral HPLC resolution conditions: column: ODH (2 × 25 cm); mobile phase: n-heptane/ethanol (0.1% ammonia water) = 50:50. |
| | 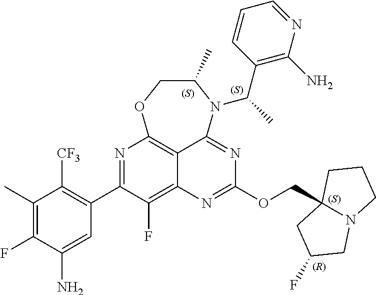 250 & 263 | | | | |
| 263 | | 3-((R or S)-1-((S)-5-(5-amino-4- | 689.3 | (400 MHz, CD₃OD) δ 7.95 (d, J = | |
| | | fluoro-3-methyl-2- | | 1.6 Hz, 1H), 7.78 (d, J = 7.4 Hz, | |
| | | (trifluoromethyl)phenyl)-4- | | 1H), 6.84-6.73 (m, 1H), 6.73-6.51 | |
| | | fluoro-2-(((2R,7aS)-2- | | (m, 2H), 5.37-5.18 (m, 1H), 4.38- | |
| | | fluorotetrahydro-1H-pyrrolizin- | | 4.20 (m, 3H), 4.03-3.86 (m, 2H), | |
| | | 7a(5H)-yl)methoxy)-9-methyl- | | 3.27-3.10 (m, 3H), 3.03-2.92 (m, | |
| | | 8,9-dihydro-10H-7-oxa-1,3,6,10- | | 1H), 2.44-2.05 (m, 6H), 2.02-1.77 | |
| | | tetraazacyclohepta[de]naphthalen- | | (m, 3H), 1.69 (d, J = 7.0 Hz, 3H), | |
| | | 10-yl)ethyl)pyridin-2-amine | | 1.25 (d, J = 6.9 Hz, 3H). | |
| | | (diastereomer 2) | | | |
| 251 | 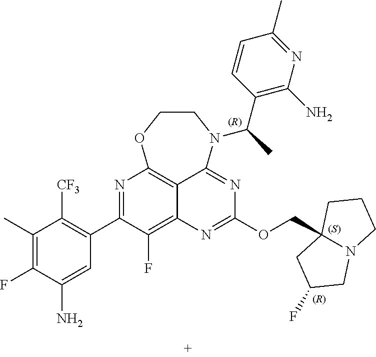 | 3-((R or S)-1-(5-(5-amino-4- fluoro-3-methyl-2- (trifluoromethyl)phenyl)-4- fluoro-2-(((2R,7aS)-2- fluorotetrahydro-1H-pyrrolizin- 7a(5H)-yl)methoxy)-8,9-dihydro- 10H-7-oxa-1,3,6,10- tetraazacyclohepta[de]naphthalen- 10-yl)ethyl)-6-methylpyridin-2- amine (diasteromer 1) | 689.3 | (400 MHz, CD₃OD) δ 7.62 (d, J = 7.7 Hz, 1H), 6.63 (d, J = 7.7 Hz, 1H), 6.60-6.52 (m, 2H), 5.43-5.17 (m, 1H), 4.48-4.25 (m, 4H), 3.79- 3.64 (m, 1H), 3.58-3.43 (m, 1H), 3.28-3.13 (m, 3H), 3.06-2.95 (m, 1H), 2.42-2.33 (m, 6H), 2.29-2.10 (m, 3H), 2.04-1.86 (m, 3H), 1.65- 1.57 (m, 3H). | It was obtained by resolving the compound prepared from I-A18, I-B1 and I-C22 by following the preparation steps of the compound 2; chiral HPLC resolution conditions: column: ODH (2 × 25 cm); mobile phase: n-heptane/ethanol (0.1% ammonia water) = 70:30. |
| | 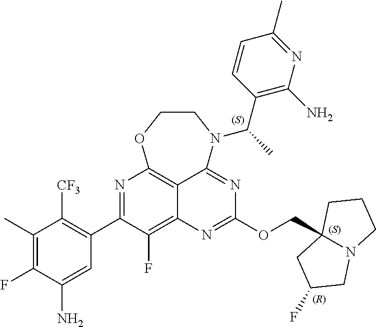 251 & 252 | | | | |
| 252 | | 3-((R or S)-1-(5-(5-amino-4- | 689.3 | (400 MHz, CD₃OD) δ 7.62 (d, J = | |
| | | fluoro-3-methyl-2- | | 7.7 Hz, 1H), 6.63 (d, J = 7.7 Hz, | |
| | | (trifluoromethyl)phenyl)-4- | | 1H), 6.60-6.52 (m, 2H), 5.45-5.19 | |
| | | fluoro-2-(((2R,7aS)-2- | | (m, 1H), 4.54-4.21 (m, 4H), 3.81- | |
| | | fluorotetrahydro-1H-pyrrolizin- | | 3.63 (m, 1H), 3.58-3.44 (m, 1H), | |
| | | 7a(5H)-yl)methoxy)-8,9-dihydro- | | 3.28-3.14 (m, 3H), 3.07-2.95 (m, | |
| | | 10H-7-oxa-1,3,6,10- | | 1H), 2.39-2.32 (m, 6H), 2.29-2.09 | |
| | | tetraazacyclohepta[de]naphthalen- | | (m, 3H), 2.06-1.85 (m, 3H), 1.65- | |
| | | 10-yl)ethyl)-6-methylpyridin-2- | | 1.57 (m, 3H). | |
| | | amine (diastereomer 2) | | | |
| 253 | 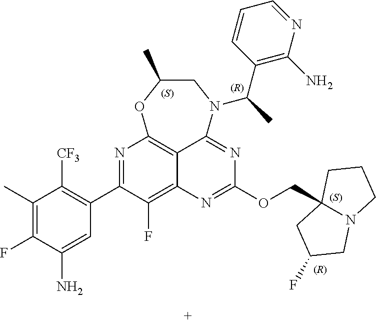 | 3-((R or S)-1-((S)-5-(5-amino-4- fluoro-3-methyl-2- (trifluoromethyl)phenyl)-4- fluoro-2-(((2R,7aS)-2- fluorotetrahydro-1H-pyrrolizin- 7a(5H)-yl)methoxy)-8-methyl- 8,9-dihydro-10H-7-oxa-1,3,6,10- tetraazacyclohepta[de]naphthalen- 10-yl)ethyl)pyridin-2-amine (diastereomer 1) | 689.3 | (400 MHz, CD₃OD) δ 7.95-7.91 (m, 1H), 7.77-7.71 (m, 1H), 6.80- 6.73 (m, 1H), 6.68-6.50 (m, 2H), 5.42-5.18 (m, 1H), 4.70-4.54 (m, 1H), 4.41-4.23 (m, 2H), 3.72-3.56 (m, 1H), 3.28-3.15 (m, 3H), 3.06- 2.96 (m, 1H), 2.41-2.11 (m, 6H), 2.03-1.85 (m, 3H), 1.70-1.60 (m, 3H), 1.33-1.24 (m, 3H). | It was obtained by resolving the compound prepared from I-A18, I-B1 and I-C23 by following the preparation steps of the compound 2; chiral HPLC resolution conditions: column: ODH (2 × 25 cm); mobile phase: n-heptane/ethanol (0.1% ammonia water) = 70:30. |
| | 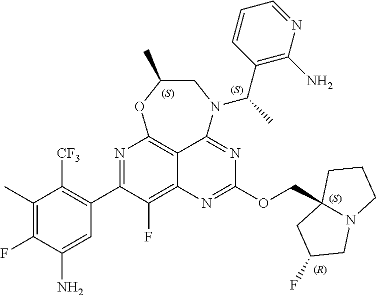 253 & 254 | | | | |
| 254 | | 3-((R or S)-1-((S)-5-(5-amino-4- | 689.3 | (400 MHz, CD₃OD) δ 8.01-7.95 | |
| | | fluoro-3-methyl-2- | | (m, 1H), 7.80-7.73 (m, 1H), 6.83- | |
| | | (trifluoromethyl)phenyl)-4- | | 6.75 (m, 1H), 6.65-6.49 (m, 2H), | |
| | | fluoro-2-(((2R,7aS)-2- | | 5.44-5.18 (m, 1H), 4.35-4.14 (m, | |
| | | fluorotetrahydro-1H-pyrrolizin- | | 3H), 3.57-3.41 (m, 2H), 3.28-3.16 | |
| | | 7a(5H)-yl)methoxy)-8-methyl- | | (m, 3H), 3.07-2.95 (m, 1H), 2.38- | |
| | | 8,9-dihydro-10H-7-oxa-1,3,6,10- | | 2.12 (m, 6H), 2.05-1.85 (m, 3H), | |
| | | tetraazacyclohepta[de]naphthalen- | | 1.66-1.53 (m, 3H), 1.20-1.13 (m, | |
| | | 10-yl)ethyl)pyridin-2-amine | | 3H). | |
| | | (diastereomer 2) | | | |
| 255 | 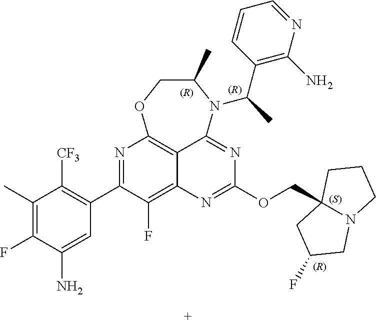 | 3-((R or S)-1-((R)-5-(5-amino-4- fluoro-3-methyl-2- (trifluoromethyl)phenyl)-4- fluoro-2-(((2R,7aS)-2- fluorotetrahydro-1H-pyrrolizin- 7a(5H)-yl)methoxy)-9-methyl- 8,9-dihydro-10H-7-oxa-1,3,6,10- tetraazacyclohepta[de]naphthalen- 10-yl)ethyl)pyridin-2-amine (diastereomer 1) | 689.3 | (400 MHz, CD₃OD) δ 8.03-7.89 (m, 1H), 7.85-7.74 (m, 1H), 6.86- 6.46 (m, 3H), 5.43-5.17 (m, 1H), 4.68-4.53 (m, 1H), 4.37-4.25 (m, 3H), 4.10-3.98 (m, 1H), 3.29-3.14 (m, 3H), 3.05-2.94 (m, 1H), 2.39- 2.35 (m, 3H), 2.34-2.08 (m, 3H), 2.03-1.85 (m, 3H), 1.70-1.60 (m, 3H), 0.81-0.56 (m, 3H). | It was obtained by resolving the compound prepared from I-A18, I-B1 and I-C24 by following the preparation steps of the compound 2; chiral HPLC resolution conditions: column: ODH (2 × 25 cm); mobile phase: n-heptane/ethanol (0.1% ammonia water) = 20:80. |
| | 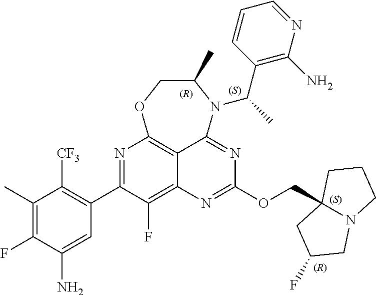 255 & 256 | | | | |
| 256 | | 3-((R or S)-1-((R)-5-(5-amino-4- | 689.3 | (400 MHz, CD₃OD) δ 8.41-8.28 | |
| | | fluoro-3-methyl-2- | | (m, 1H), 7.91-7.74 (m, 1H), 7.30- | |
| | | (trifluoromethyl)phenyl)-4- | | 7.11 (m, 1H), 6.77-6.54 (m, 1H), | |
| | | fluoro-2-(((2R,7aS)-2- | | 5.40-5.14 (m, 1H), 4.72-4.68 (m, | |
| | | fluorotetrahydro-1H-pyrrolizin- | | 1H), 4.41-4.19 (m, 2H), 4.09-3.80 | |
| | | 7a(5H)-yl)methoxy)-9-methyl- | | (m, 2H), 3.29-3.09 (m, 4H), 3.03- | |
| | | 8,9-dihydro-10H-7-oxa-1,3,6,10- | | 2.91 (m, 1H), 2.42-2.37 (m, 3H), | |
| | | tetraazacyclohepta[de]naphthalen- | | 2.37-2.05 (m, 3H), 2.03-1.77 (m, | |
| | | 10-yl)ethyl)pyridin-2-amine | | 3H), 1.69-1.54 (m, 3H), 1.31-1.22 | |
| | | (diastereomer 2) | | (m, 3H). | |
| 257 | 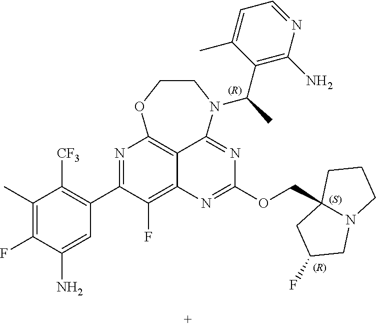 | 3-((R or S)-1-(5-(5-amino-4- fluoro-3-methyl-2- (trifluoromethyl)phenyl)-4- fluoro-2-(((2R,7aS)-2- fluorotetrahydro-1H-pyrrolizin- 7a(5H)-yl)methoxy)-8,9-dihydro- 10H-7-oxa-1,3,6,10- tetraazacyclohepta[de]naphthalen- 10-yl)ethyl)-4-methylpyridin-2- amine (diastereomer 1) | 689.3 | (400 MHz, CD₃OD) δ 7.81-7.76 (m, 1H), 6.61-6.54 (m, 2H), 6.42- 6.30 (m, 1H), 5.38-5.20 (m, 1H), 4.57-4.37 (m, 2H), 4.29-4.14 (m, 2H), 4.03-3.93 (m, 1H), 3.84-3.72 (m, 1H), 3.27-3.12 (m, 3H), 3.05- 2.95 (m, 1H), 2.41-2.34 (m, 6H), 2.32-2.08 (m, 3H), 2.02-1.84 (m, 3H), 1.80-1.70 (m, 3H). | It was obtained by resolving the compound prepared from I-A18, I-B1 and I-C25 by following the preparation steps of the compound 2; chiral HPLC resolution conditions: column: IC (2 × 25 cm); mobile phase: n-heptane/ethanol (0.1% ammonia water) = 50:50. |
| | 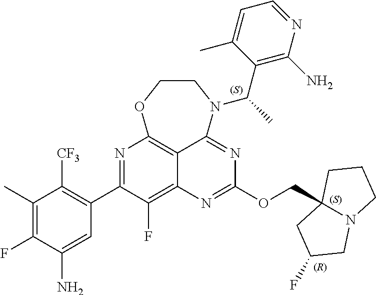 257 & 258 | | | | |
| 258 | | 3-((R or S)-1-(5-(5-amino-4- | 689.3 | (400 MHz, CD₃OD) δ 7.81-7.74 | |
| | | fluoro-3-methyl-2- | | (m, 1H), 6.62-6.52 (m, 2H), 6.40- | |
| | | (trifluoromethyl)phenyl)-4- | | 6.32 (m, 1H), 5.39-5.21 (m, 1H), | |
| | | fluoro-2-(((2R,7aS)-2- | | 4.58-4.37 (m, 2H), 4.34-4.11 (m, | |
| | | fluorotetrahydro-1H-pyrrolizin- | | 2H), 4.02-3.92 (m, 1H), 3.84-3.72 | |
| | | 7a(5H)-yl)methoxy)-8,9-dihydro- | | (m, 1H), 3.29-3.13 (m, 3H), 3.05- | |
| | | 10H-7-oxa-1,3,6,10- | | 2.95 (m, 1H), 2.41-2.35 (m, 6H), | |
| | | tetraazacyclohepta[de]naphthalen- | | 2.33-2.07 (m, 3H), 2.02-1.83 (m, | |
| | | 10-yl)ethyl)-4-methylpyridin-2- | | 3H), 1.78-1.71 (m, 3H). | |
| | | amine (diastereomer 2) | | | |
| 260 | 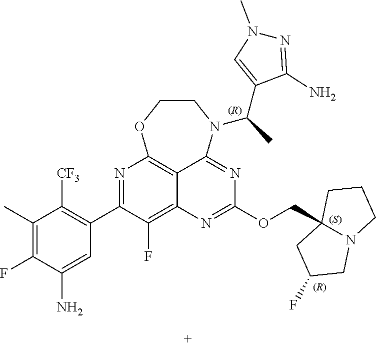 | 4-((R or S)-1-(5-(5-amino-4- fluoro-3-methyl-2- (trifluoromethyl)phenyl)-4- fluoro-2-(((2R,7aS)-2- fluorotetrahydro-1H-pyrrolizin- 7a(5H)-yl)methoxy)-8,9-dihydro- 10H-7-oxa-1,3,6,10- tetraazacyclohepta[de]naphthalen- 10-yl)ethyl)-1-methyl-1H- pyrazol-3-amine (diastereomer 1) | 678.3 | (400 MHz, CD₃OD) δ 7.52-7.46 (m, 1H), 6.60-6.53 (m, 1H), 6.53- 6.43 (m, 1H), 5.36-5.20 (m, 1H), 4.52-4.21 (m, 4H), 3.85-3.63 (m, 5H), 3.27-3.10 (m, 3H), 3.02-2.95 (m, 1H), 2.38-2.33 (m, 3H), 2.33- 2.10 (m, 3H), 2.02-1.84 (m, 3H), 1.57-1.50 (m, 3H). | It was obtained by resolving the compound prepared from I-A18, I-B1 and I-C26 by following the preparation steps of the compound 2; chiral HPLC resolution conditions: column: ODH (2 × 25 cm); mobile phase: n-heptane/ethanol (0.1% ammonia water) = 70:30. |
| | 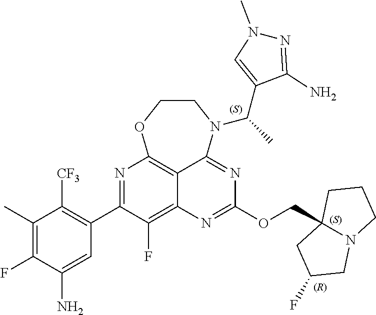 260 & 261 | | | | |
| 261 | | 4-((R or S)-1-(5-(5-amino-4- | 678.3 | (400 MHz, CD₃OD) δ 7.52-7.46 | |
| | | fluoro-3-methyl-2- | | (m, 1H), 6.61-6.52 (m, 1H), 6.52- | |
| | | (trifluoromethyl)phenyl)-4- | | 6.43 (m, 1H), 5.37-5.21 (m, 1H), | |
| | | fluoro-2-(((2R,7aS)-2- | | 4.51-4.25 (m, 4H), 3.73-3.63 (m, | |
| | | fluorotetrahydro-1H-pyrrolizin- | | 5H), 3.27-3.11 (m, 3H), 3.02-2.95 | |
| | | 7a(5H)-yl)methoxy)-8,9-dihydro- | | (m, 1H), 2.39-2.07 (m, 6H), 2.02- | |
| | | 10H-7-oxa-1,3,6,10- | | 1.81 (m, 3H), 1.57-1.49 (m, 3H). | |
| | | tetraazacyclohepta[de]naphthalen- | | | |
| | | 10-yl)ethyl)-1-methyl-1H- | | | |
| | | pyrazol-3-amine (diastereomer 2) | | | |
| 264 | 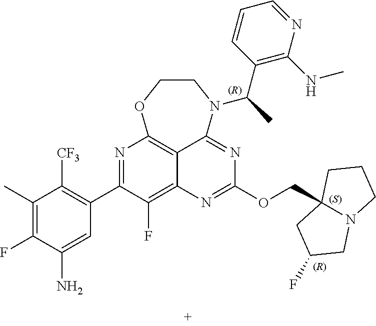 | 3-((R or S)-1-(5-(5-amino-4- fluoro-3-methyl-2- (trifluoromethyl)phenyl)-4- fluoro-2-(((2R,7aS)-2- fluorotetrahydro-1H-pyrrolizin- 7a(5H)-yl)methoxy)-8,9-dihydro- 10H-7-oxa-1,3,6,10- tetraazacyclohepta[de]naphthalen- 10-yl)ethyl)-N-methylpyridin-2- amine (diastereomer 1) | 689.2 | (400 MHz, CD₃OD) δ 8.09-7.95 (m, 1H), 7.71-7.63 (m, 1H), 6.72- 6.64 (m, 1H), 6.64-6.39 (m, 2H), 5.41-5.12 (m, 1H), 4.47-4.38 (m, 1H), 4.34-4.17 (m, 3H), 3.83-3.65 (m, 1H), 4.34-4.17 (m, 3H), 3.27- 3.09 (m, 3H), 3.04-2.98 (m, 1H), 2.88 (s, 3H), 2.39-2.34 (m, 3H), 2.33-2.08 (m, 3H), 2.05-1.79 (m, 3H), 1.67-1.56 (m, 3H). | It was obtained by resolving the compound prepared from I-A18, I-B1 and I-C27 by following the preparation steps of the compound 2; chiral HPLC resolution conditions: column: ODH (2 × 25 cm); mobile phase: n-heptane/ethanol (0.1% ammonia water) = 70:30. |
| | 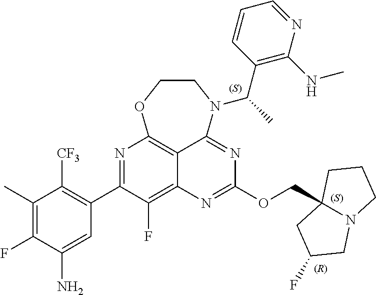 264 & 265 | | | | |
| 265 | | 3-((R or S)-1-(5-(5-amino-4- | 689.2 | (400 MHz, CD₃OD) δ 8.07-7.95 | |
| | | fluoro-3-methyl-2- | | (m, 1H), 7.73-7.60 (m, 1H), 6.76- | |
| | | (trifluoromethyl)phenyl)-4- | | 6.63 (m, 1H), 6.62-6.43 (m, 2H), | |
| | | fluoro-2-(((2R,7aS)-2- | | 5.42-5.17 (m, 1H), 4.53-4.32 (m, | |
| | | fluorotetrahydro-1H-pyrrolizin- | | 2H), 4.31-4.16 (m, 2H), 3.82-3.66 | |
| | | 7a(5H)-yl)methoxy)-8,9-dihydro- | | (m, 1H), 3.58-3.43 (m, 1H), 3.26- | |
| | | 10H-7-oxa-1,3,6,10- | | 3.11 (m, 3H), 3.07-2.94 (m, 1H), | |
| | | tetraazacyclohepta[de]naphthalen- | | 2.91-2.81 (m, 3H), 2.39-2.32 (m, | |
| | | 10-yl)ethyl)-N-methylpyridin-2- | | 3H), 2.31-2.04 (m, 3H), 2.04-1.78 | |
| | | amine (diastereomer 2) | | (m, 3H), 1.68-1.55 (m, 3H). | |
| 269 | 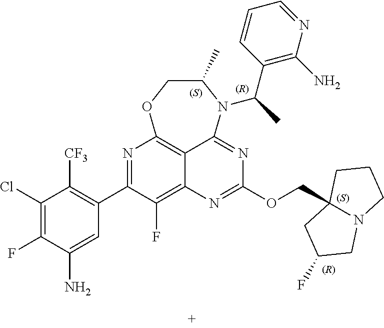 | 3-((R or S)-1-((S)-5-(5-amino-3- chloro-4-fluoro-2- (trifluoromethyl)phenyl)-4- fluoro-2-(((2R,7aS)-2- fluorotetrahydro-1H-pyrrolizin- 7a(5H)-yl)methoxy)-9-methyl- 8,9-dihydro-10H-7-oxa-1,3,6,10- tetraazacyclohepta[de]naphthalen- 10-yl)ethyl)pyridin-2-amine (diastereomer 1) | 709.2 | (400 MHz, CD₃OD) δ 8.11-7.91 (m, 1H), 7.84-7.71 (m, 1H), 6.86- 6.58 (m, 3H), 5.48-5.09 (m, 1H), 4.64-4.58 (m, 1H), 4.35-4.22 (m, 3H), 4.10-3.98 (m, 1H), 3.26-3.13 (m, 3H), 3.04-2.93 (m, 1H), 2.43- 2.09 (m, 3H), 2.01-1.84 (m, 3H), 1.65 (d, J = 6.8 Hz, 3H), 0.66 (d, J = 6.8 Hz, 3H). | It was obtained by resolving the compound prepared from I-A28, I-B1 and I-C21 by following the preparation steps of the compound 2; chiral HPLC resolution conditions: column: ODH (2 × 25 cm); mobile phase: n-heptane/ethanol (0.1% ammonia water) = 50:50. |
| | 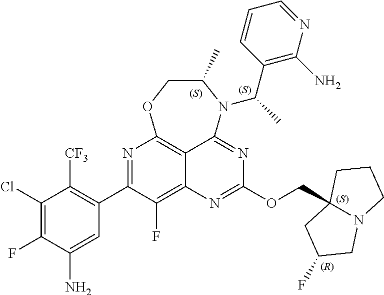 269 & 272 | | | | |
| 272 | | 3-((R or S)-1-((S)-5-(5-amino-3- | 709.2 | (400 MHz, CD₃OD) δ 7.99-7.92 | |
| | | chloro-4-fluoro-2- | | (m, 1H), 7.84-7.76 (m, 1H), 6.82- | |
| | | (trifluoromethyl)phenyl)-4- | | 6.76 (m, 1H), 6.75-6.62 (m, 2H), | |
| | | fluoro-2-(((2R,7aS)-2- | | 5.38-5.21 (m, 1H), 4.37-4.29 (m, | |
| | | fluorotetrahydro-1H-pyrrolizin- | | 2H), 4.27-4.20 (m, 1H), 4.05-3.91 | |
| | | 7a(5H)-yl)methoxy)-9-methyl- | | (m, 2H), 3.26-3.10 (m, 3H), 3.03- | |
| | | 8,9-dihydro-10H-7-oxa-1,3,6,10- | | 2.93 (m, 1H), 2.35-2.07 (m, 3H), | |
| | | tetraazacyclohepta[de]naphthalen- | | 2.05-1.86 (m, 3H), 1.70 (d, J = | |
| | | 10-yl)ethyl)pyridin-2-amine | | 7.0 Hz, 3H), 1.26 (d, J = 6.9 Hz, | |
| | | (diastereomer 2) | | 3H). | |
| 270 | 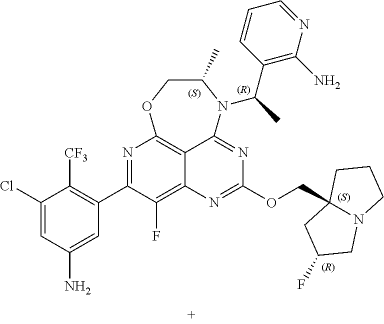 | 3-((R or S)-1-((S)-5-(5-amino-3- chloro-2- (trifluoromethyl)phenyl)-4- fluoro-2-(((2R,7aS)-2- fluorotetrahydro-1H-pyrrolizin- 7a(5H)-yl)methoxy)-9-methyl- 8,9-dihydro-10H-7-oxa-1,3,6,10- tetraazacyclohepta[de]naphthalen- 10-yl)ethyl)pyridin-2-amine (diastereomer 1) | 691.3 | (400 MHz, CD₃OD) δ 7.97-7.93 (m, 1H), 7.80-7.76 (m, 1H), 6.89- 6.68 (m, 3H), 6.59-6.37 (m, 1H), 5.38-5.21 (m, 1H), 4.63-4.56 (m, 1H), 4.36-4.24 (m, 3H), 4.08-4.00 (m, 1H), 3.28-3.17 (m, 3H), 3.05- 2.98 (m, 1H), 2.40-2.13 (m, 3H), 2.03-1.86 (m, 3H), 1.68-1.62 (m, 3H), 0.69-0.64 (m, 3H). | It was obtained by resolving the compound prepared from I-A14, I-B1 and I-C21 by following the preparation steps of the compound 2; chiral HPLC resolution conditions: column: ODH (2 × 25 cm); mobile phase: n-heptane/ethanol (0.1% ammonia water) = 70:30. |
| | 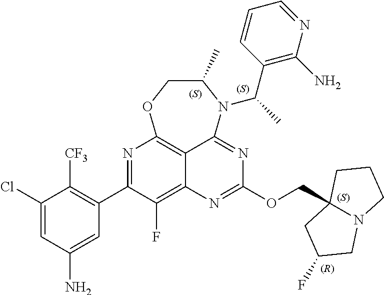 270 & 271 | | | | |
| 271 | | 3-((R or S)-1-((S)-5-(5-amino-3- | 691.2 | (400 MHz, CD₃OD) δ 7.98-7.93 | |
| | | chloro-2- | | (m, 1H), 7.82-7.76 (m, 1H), 6.89- | |
| | | (trifluoromethyl)phenyl)-4- | | 6.62 (m, 3H), 6.57-6.41 (m, 1H), | |
| | | fluoro-2-(((2R,7aS)-2- | | 5.39-5.18 (m, 1H), 4.39-4.20 (m, | |
| | | fluorotetrahydro-1H-pyrrolizin- | | 3H), 4.06-3.91 (m, 2H), 3.28-3.11 | |
| | | 7a(5H)-yl)methoxy)-9-methyl- | | (m, 3H), 3.03-2.94 (m, 1H), 2.40- | |
| | | 8,9-dihydro-10H-7-oxa-1,3,6,10- | | 2.08 (m, 3H), 2.01-1.81 (m, 3H), | |
| | | tetraazacyclohepta[de]naphthalen- | | 1.73-1.66 (m, 3H), 1.27-1.23 (m, | |
| | | 10-yl)ethyl)pyridin-2-amine | | 3H). | |
| | | (diastereomer 2) | | | |
| 273 | 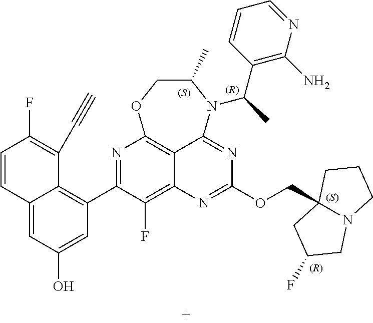 | 4-((S)-10-((R or S)-1-(2- aminopyridin-3-yl)ethyl)-4- fluoro-2-(((2R,7aS)-2- fluorotetrahydro-1H-pyrrolizin- 7a(5H)-yl)methoxy)-9-methyl- 9,10-dihydro-8H-7-oxa-1,3,6,10- tetraazacyclohepta[de]naphthalen- 5-yl)-5-ethynyl-6- fluoronaphthalen-2-ol (diastereomer 1) | 682.3 | (400 MHz, CD₃OD) δ 8.00-7.94 (m, 1H), 7.84-7.78 (m, 2H), 7.32- 7.25 (m, 2H), 7.25-7.10 (m, 1H), 6.81-6.73 (m, 2H), 5.40-5.19 (m, 1H), 4.65-4.57 (m, 1H), 4.36-4.24 (m, 3H), 4.11-4.03 (m, 1H), 3.54- 3.52 (m, 1H), 3.27-3.15 (m, 3H), 3.04-2.97 (m, 1H), 2.39-2.11 (m, 3H), 2.03-1.85 (m, 3H), 1.72-1.64 (m, 3H), 0.75-0.67 (m, 3H). | It was obtained by resolving the compound prepared from I-A30, I-B1 and I-C21 by following the preparation steps of the compound 2; chiral HPLC resolution conditions: column: ODH (2 × 25 cm); mobile phase: n-heptane/ethanol (0.1% ammonia water) = 70:30. |
| | 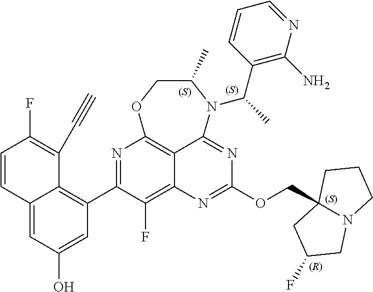 273 & 274 | | | | |
| 274 | | 4-((S)-10-((R or S)-1-(2- | 682.3 | (400 MHz, CD₃OD) δ 8.00-7.93 | |
| | | aminopyridin-3-yl)ethyl)-4- | | (m, 1H), 7.85-7.79 (m, 2H), 7.33- | |
| | | fluoro-2-(((2R,7aS)-2- | | 7.26 (m, 2H), 7.25-7.11 (m, 1H), | |
| | | fluorotetrahydro-1H-pyrrolizin- | | 6.82-6.77 (m, 1H), 6.76-.68 (m, | |
| | | 7a(5H)-yl)methoxy)-9-methyl- | | 1H), 5.38-5.21 (m, 1H), 4.38-4.22 | |
| | | 9,10-dihydro-8H-7-oxa-1,3,6,10- | | (m, 3H), 4.04-3.94 (m, 2H), 3.55- | |
| | | tetraazacyclohepta[de]naphthalen- | | 3.39 (m, 1H), 3.27-3.15 (m, 3H), | |
| | | 5-yl)-5-ethynyl-6- | | 3.04-2.95 (m, 1H), 2.40-2.09 (m, | |
| | | fluoronaphthalen-2-ol | | 3H), 2.02-1.82 (m, 3H), 1.78-1.69 | |
| | | (diastereomer 2) | | (m, 3H), 1.35-1.29 (m, 3H). | |
| 275 | 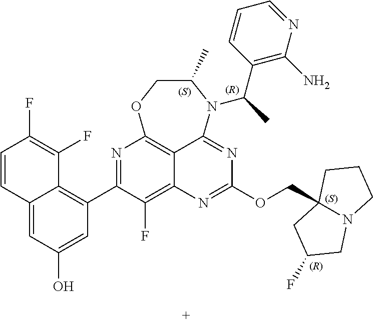 | 4-((S)-10-((R or S)-1-(2- aminopyridin-3-yl)ethyl)-4- fluoro-2-(((2R,7aS)-2- fluorotetrahydro-1H-pyrrolizin- 7a(5H)-yl)methoxy)-9-methyl- 9,10-dihydro-8H-7-oxa-1,3,6,10- tetraazacyclohepta[de]naphthalen- 5-yl)-5,6-difluoronaphthalen-2- ol (diastereomer 1) | 676.3 | (400 MHz, CD₃OD) δ 7.96 (d, J = 5.1 Hz, 1H), 7.85-7.73 (m, 1H), 7.62-7.52 (m, 1H), 7.42-7.30 (m, 1H), 7.30-7.09 (m, 2H), 6.85-6.63 (m, 2H), 5.42-5.18 (m, 1H), 4.63- 4.59 (m, 1H), 4.38-4.20 (m, 3H), 4.13-4.00 (m, 1H), 3.28-3.15 (m, 3H), 3.05-2.95 (m, 1H), 2.42-2.11 (m, 3H), 2.04-1.86 (m, 3H), 1.67 (d, J = 6.8 Hz, 3H), 0.80-0.60 (m, 3H). | It was obtained by resolving the compound prepared from I-A37, I-B1 and I-C21 by following the preparation steps of the compound 2; chiral HPLC resolution conditions: column: IC (2 × 25 cm); mobile phase: n-heptane/ethanol (0.1% ammonia water) = 50:50. |
| | 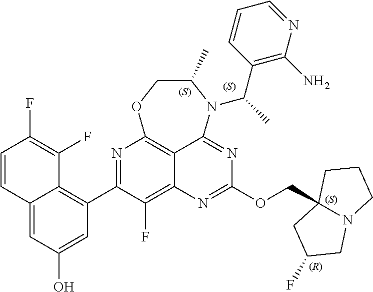 275 & 276 | | | | |
| 276 | | 4-((S)-10-((R or S)-1-(2- | 676.3 | (400 MHz, CD₃OD) δ 7.96 (d, J = | |
| | | aminopyridin-3-yl)ethyl)-4- | | 1.6 Hz, 1H), 7.86-7.78 (m, 1H), | |
| | | fluoro-2-(((2R,7aS)-2- | | 7.60-7.51 (m, 1H), 7.42-7.29 (m, | |
| | | fluorotetrahydro-1H-pyrrolizin- | | 1H), 7.29-7.12 (m, 2H), 6.85-6.66 | |
| | | 7a(5H)-yl)methoxy)-9-methyl- | | (m, 2H), 5.37-5.19 (m, 1H), 4.41- | |
| | | 9,10-dihydro-8H-7-oxa-1,3,6,10- | | 4.21 (m, 3H), 4.09-3.93 (m, 2H), | |
| | | tetraazacyclohepta[de]naphthalen- | | 3.27-3.12 (m, 3H), 3.06-2.90 (m, | |
| | | 5-yl)-5,6-difluoronaphthalen-2- | | 1H), 2.41-2.07 (m, 3H), 2.05-1.78 | |
| | | ol (diastereomer 2) | | (m, 3H), 1.77-1.65 (m, 3H), 1.35- | |
| | | | | 1.27 (m, 3H). | |
| 277 | 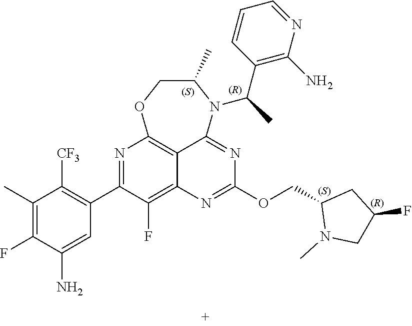 | 3-((R or S)-1-((S)-5-(5-amino-4- fluoro-3-methyl-2- (trifluoromethyl)phenyl)-4- fluoro-2-(((2S,4R)-4-fluoro-1- methylpyrrolidin-2-yl)methoxy)- 9-methyl-8,9-dihydro-10H- 7-oxa-1,3,6,10- tetraazacyclohepta[de]naphthalen- 10-yl)ethyl)pyridin-2-amine (diastereomer 1) | 663.4 | (400 MHz, CD₃OD) δ 8.00-7.90 (m, 1H), 7.86-7.75 (m, 1H), 6.84- 6.75 (m, 1H), 6.75-6.66 (m, 1H), 6.66-6.50 (m, 1H), 5.27-5.09 (m, 1H), 4.59-4.52 (m, 2H), 4.41-4.20 (m, 1H), 4.08-3.86 (m, 2H), 3.59- 3.44 (m, 1H), 3.24-3.13 (m, 1H), 2.77-2.61 (m, 1H), 2.56 (s, 3H), 2.40-2.22 (m, 4H), 2.15-1.92 (m, 1H), 1.77-1.60 (m, 3H), 1.28-1.23 (m, 3H). | It was obtained by resolving the compound prepared from I-A18, I-B10 and I-C21 by following the preparation steps of the compound 2; chiral HPLC resolution conditions: column: ODH (2 × 25 cm); mobile phase: n-heptane/ethanol (0.1% ammonia water) = 70:30. |
| | 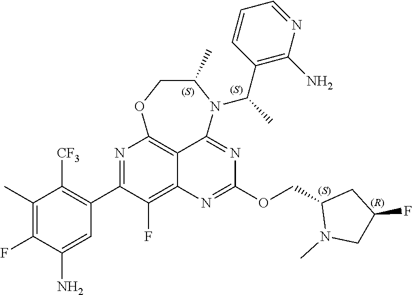 277 & 278 | | | | |
| 278 | | 3-((R or S)-1-((S)-5-(5-amino-4- | 663.3 | (400 MHz, CD₃OD) δ 8.28-8.18 | |
| | | fluoro-3-methyl-2- | | (m, 1H), 8.12-8.02 (m, 1H), 7.13- | |
| | | (trifluoromethyl)phenyl)-4- | | 6.69 (m, 3H), 5.57-5.37 (m, 1H), | |
| | | fluoro-2-(((2S,4R)-4-fluoro-1- | | 4.93-4.79 (m, 3H), 4.61-4.51 (m, | |
| | | methylpyrrolidin-2-yl)methoxy)- | | 1H), 4.38-4.27 (m, 1H), 3.96-3.72 | |
| | | 9-methyl-8,9-dihydro-10H-7-oxa- | | (m, 1H), 3.11-2.95 (m, 1H), 2.89 | |
| | | 1,3,6,10- | | (s, 3H), 2.69-2.50 (m, 4H), 2.39- | |
| | | tetraazacyclohepta[de]naphthalen- | | 2.29 (m, 2H), 2.00-1.86 (m, 3H), | |
| | | 10-yl)ethyl)pyridin-2-amine | | 1.00-0.88 (m, 3H). | |
| | | (diastereomer 2) | | | |
| 279 | 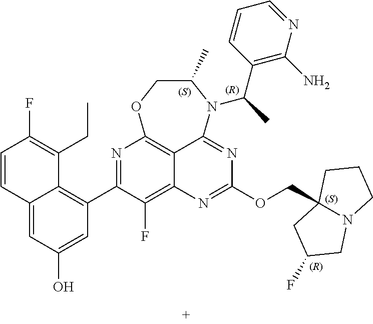 | 4-((S)-10-((R or S)-1-(2- aminopyridin-3-yl)ethyl)-4- fluoro-2-(((2R,7aS)-2- fluorotetrahydro-1H-pyrrolizin- 7a(5H)-yl)methoxy)-9-methyl- 9,10-dihydro-8H-7-oxa-1,3,6,10- tetraazacyclohepta[de]naphthalen- 5-yl)-5-ethyl-6- fluoronaphthalen-2-ol (diastereomer 1) | 686.3 | (400 MHz, CD₃OD) δ 8.01-7.89 (m, 1H), 7.80-7.73 (m, 1H), 7.65- 7.59 (m, 1H), 7.28-7.15 (m, 2H), 7.14-6.96 (m, 1H), 6.79-6.72 (m, 2H), 5.39-5.19 (m, 1H), 4.65-4.55 (m, 1H), 4.37-4.24 (m, 3H), 4.10- 4.00 (m, 1H), 3.27-3.18 (m, 2H), 3.05-2.97 (m, 1H), 2.40-2.14 (m, 4H), 2.03-1.85 (m, 3H), 1.68-1.60 (m, 3H), 0.91-0.78 (m, 3H), 0.73- 0.65 (m, 3H). | It was obtained by resolving the compound prepared from I-A38, I-B1 and I-C21 by following the preparation steps of the compound 2; chiral HPLC resolution conditions: column: ODH (2 × 25 cm); mobile phase: n-heptane/ethanol (0.1% ammonia water) = 70:30. |
| | 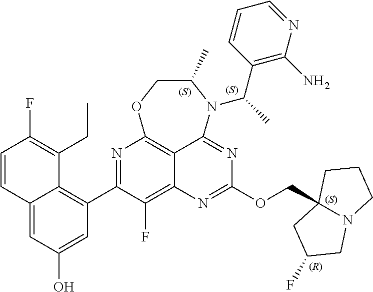 279 & 280 | | | | |
| 280 | | 4-((S)-10-((R or S)-1-(2- | 686.3 | (400 MHz, CD₃OD) δ 7.99-7.93 | |
| | | aminopyridin-3-yl)ethyl)-4- | | (m, 1H), 7.82-7.77 (m, 1H), 7.65- | |
| | | fluoro-2-(((2R,7aS)-2- | | 7.60 (m, 1H), 7.27-7.18 (m, 2H), | |
| | | fluorotetrahydro-1H-pyrrolizin- | | 7.10-6.99 (m, 1H), 6.80-6.75 (m, | |
| | | 7a(5H)-yl)methoxy)-9-methyl- | | 1H), 6.74-6.66 (m, 1H), 5.40-5.21 | |
| | | 9,10-dihydro-8H-7-oxa-1,3,6,10- | | (m, 1H), 4.40-4.25 (m, 3H), 4.06- | |
| | | tetraazacyclohepta[de]naphthalen- | | 3.92 (m, 2H), 3.27-3.20 (m, 2H), | |
| | | 5-yl)-5-ethyl-6- | | 3.07-2.98 (m, 1H), 2.43-2.11 (m, | |
| | | fluoronaphthalen-2-ol | | 4H), 2.04-1.83 (m, 3H), 1.74-1.68 | |
| | | (diastereomer 2) | | (m, 3H), 1.31-1.27 (m, 3H), 0.88- | |
| | | | | 0.83 (m, 3H). | |
| 282 | 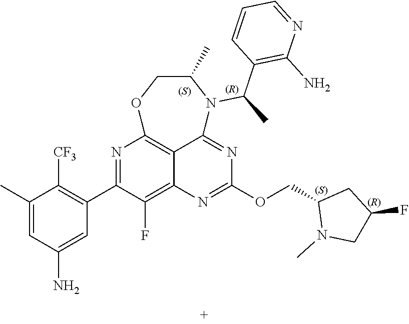 | 3-((R or S)-1-((S)-5-(5-amino-3- methyl-2- (trifluoromethyl)phenyl)-4- fluoro-2-(((2S,4R)-4-fluoro-1- methylpyrrolidin-2-yl)methoxy)- 9-methyl-8,9-dihydro-10H- 7-oxa-1,3,6,10- tetraazacyclohepta[de]naphthalen- 10-yl)ethyl)pyridin-2-amine (diastereomer 1) | 645.3 | (400 MHz, CD₃OD) δ 7.98-7.92 (m, 1H), 7.80-7.75 (m, 1H), 6.78- 6.71 (m, 2H), 6.70-6.63 (m, 1H), 6.52-6.30 (m, 1H), 5.26-5.07 (m, 1H), 4.62-4.55 (m, 2H), 4.55-4.50 (m, 1H), 4.32-4.22 (m, 1H), 4.08- 3.99 (m, 1H), 3.57-3.42 (m, 1H), 3.21-3.13 (m, 1H), 2.72-2.59 (m, 1H), 2.55 (s, 3H), 2.35-2.22 (m, 1H), 2.11-1.94 (m, 1H), 1.69-1.61 (m, 3H), 0.69-0.62 (m, 3H). | It was obtained by resolving the compound prepared from I-A12, I-B10 and I-C21 by following the preparation steps of the compound 2; chiral HPLC resolution conditions: column: ODH (2 × 25 cm); mobile phase: n-heptane/ethanol (0.1% ammonia water) = 70:30. |
| | 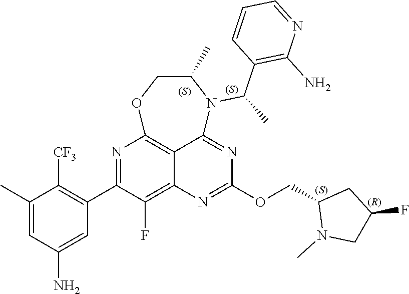 282 & 283 | | | | |
| 283 | | 3-((R or S)-1-((S)-5-(5-amino-3- | 645.3 | (400 MHz, CD₃OD) δ 7.99-7.91 | |
| | | methyl-2- | | (m, 1H), 7.82-7.77 (m, 1H), 6.80- | |
| | | (trifluoromethyl)phenyl)-4- | | 6.76 (m, 1H), 6.74-6.64 (m, 2H), | |
| | | fluoro-2-(((2S,4R)-4-fluoro-1- | | 6.50-6.33 (m, 1H), 5.25-5.07 (m, | |
| | | methylpyrrolidin-2-yl)methoxy)- | | 1H), 4.58-4.48 (m, 2H), 4.34-4.27 | |
| | | 9-methyl-8,9-dihydro-10H-7-oxa- | | (m, 1H), 4.02-3.90 (m, 2H), 3.55- | |
| | | 1,3,6,10- | | 3.43 (m, 1H), 3.19-3.09 (m, 1H), | |
| | | tetraazacyclohepta[de]naphthalen- | | 2.70-2.58 (m, 1H), 2.54 (s, 3H), | |
| | | 10-yl)ethyl)pyridin-2-amine | | 2.34-2.22 (m, 1H), 2.10-1.93 (m, | |
| | | (diastereomer 2) | | 1H), 1.72-1.67 (m, 3H), 1.27-1.24 | |
| | | | | (m, 3H). | |
| 284 | 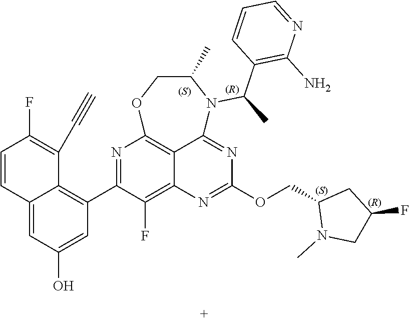 | 4-((S)-10-((R or S)-1-(2- aminopyridin-3-yl)ethyl)-4- fluoro-2-(((2S,4R)-4-fluoro-1- methylpyrrolidin-2-yl)methoxy)- 9-methyl-9,10-dihydro-8H- 7-oxa-1,3,6,10- tetraazacyclohepta[de]naphthalen- 5-yl)-5-ethynyl-6- fluoronaphthalen-2-ol (diastereomer 1) | 656.3 | (400 MHz, CD₃OD) δ 7.99-7.91 (m, 1H), 7.84-7.75 (m, 2H), 7.32- 7.11 (m, 3H), 6.80-6.71 (m, 2H), 5.27-5.05 (m, 1H), 4.64-4.50 (m, 3H), 4.33-4.24 (m, 1H), 4.09-4.00 (m, 1H), 3.56 (s, 1H), 3.55-3.42 (m, 1H), 3.21-3.09 (m, 1H), 2.70- 2.57 (m, 1H), 2.57-2.49 (m, 3H), 2.35-2.21 (m, 1H), 2.10-1.92 (m, 1H), 1.70-1.60 (m, 3H), 0.76-0.65 (m, 3H). | It was obtained by resolving the compound prepared from I-A30, I-B10 and I-C21 by following the preparation steps of the compound 2; chiral HPLC resolution conditions: column: ODH (2 × 25 cm); mobile phase: n-heptane/ethanol (0.1% ammonia water) = 70:30. |
| | 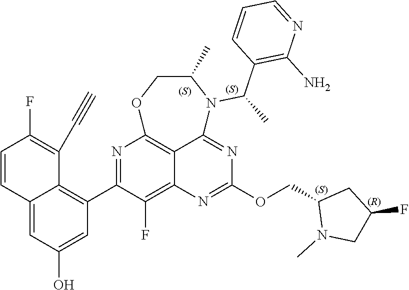 284 & 285 | | | | |
| 285 | | 4-((S)-10-((R or S)-1-(2- | 656.3 | (400 MHz, CD₃OD) δ 7.99-7.91 | |
| | | aminopyridin-3-yl)ethyl)-4- | | (m, 1H), 7.84-7.77 (m, 2H), 7.32- | |
| | | fluoro-2-(((2S,4R)-4-fluoro-1- | | 7.15 (m, 3H), 6.80-6.68 (m, 2H), | |
| | | methylpyrrolidin-2-yl)methoxy)- | | 5.27-5.06 (m, 1H), 4.58-4.47 (m, | |
| | | 9-methyl-9,10-dihydro-8H-7-oxa- | | 2H), 4.37-4.27 (m, 1H), 4.03-3.92 | |
| | | 1,3,6,10- | | (m, 2H), 3.56-3.42 (m, 2H), 3.18- | |
| | | tetraazacyclohepta[de]naphthalen- | | 3.09 (m, 1H), 2.70-2.57 (m, 1H), | |
| | | 5-yl)-5-ethynyl-6- | | 2.53 (s, 3H), 2.35-2.21 (m, 1H), | |
| | | fluoronaphthalen-2-ol | | 2.11-1.93 (m, 1H), 1.75-1.67 (m, | |
| | | (diastereomer 2) | | 3H), 1.34-1.27 (m, 3H). | |
| 286 | 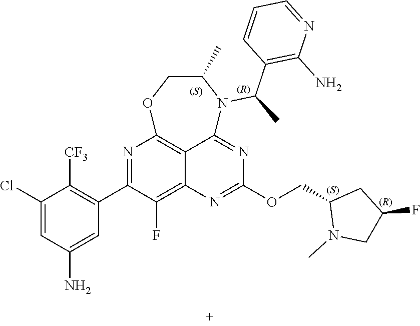 | 3-((R or S)-1-((S)-5-(5-amino-3- chloro-2- (trifluoromethyl)phenyl)-4- fluoro-2-(((2S,4R)-4-fluoro-1- methylpyrrolidin-2-yl)methoxy)- 9-methyl-8,9-dihydro-1H- 7-oxa-1,3,6,10- tetraazacyclohepta[de]naphthalen- 10-yl)ethyl)pyridin-2-amine (diastereomer 1) | 665.2 | (400 MHz, CD₃OD) δ 8.01-7.95 (m, 1H), 7.83 (d, J = 7.3 Hz, 1H), 6.91-6.86 (m, 1H), 6.85-6.77 (m, 1H), 6.77-6.70 (m, 1H), 6.50 (d, J = 33.1 Hz, 1H), 5.20 (d, J = 55.3 Hz, 1H), 4.61-4.54 (m, 2H), 4.38- 4.30 (m, 1H), 4.04-3.95 (m, 2H), 3.60-3.45 (m, 1H), 3.22-3.14 (m, 1H), 2.74-2.62 (m, 1H), 2.57 (s, 3H), 2.39-2.24 (m, 1H), 2.14-1.96 (m, 1H), 1.72 (d, J = 7.0 Hz, 3H), 1.28 (d, J = 6.9 Hz, 3H). | It was obtained by resolving the compound prepared from I-A14, I-B10 and I-C21 by following the preparation steps of the compound 2; chiral HPLC resolution conditions: column: ODH (2 × 25 cm); mobile phase: n-heptane/ethanol (0.1% ammonia water) = 70:30. |
| | 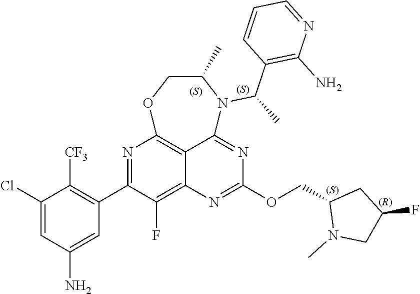 286 & 287 | | | | |
| 287 | | 3-((R or S)-1-((S)-5-(5-amino-3- | 665.2 | (400 MHz, CD₃OD) δ 7.99-7.93 | |
| | | chloro-2- | | (m, 1H), 7.79 (d, J = 7.5 Hz, 1H), | |
| | | (trifluoromethyl)phenyl)-4- | | 6.89-6.83 (m, 1H), 6.81-6.71 (m, | |
| | | fluoro-2-(((2S,4R)-4-fluoro-1- | | 2H), 6.47 (d, J = 50.6 Hz, 1H), | |
| | | methylpyrrolidin-2-yl)methoxy)- | | 5.17 (d, J = 55.5 Hz, 1H), 4.63- | |
| | | 9-methyl-8,9-dihydro-10H-7-oxa- | | 4.56 (m, 2H), 4.55-4.50 (m, 1H), | |
| | | 1,3,6,10- | | 4.35-4.24 (m, 1H), 4.08-4.01 (m, | |
| | | tetraazacyclohepta[de]naphthalen- | | 1H), 3.57-3.41 (m, 1H), 3.20-3.11 | |
| | | 10-yl)ethyl)pyridin-2-amine | | (m, 1H), 2.71-2.58 (m, 1H), 2.55 | |
| | | (diastereomer 2) | | (s, 3H), 2.37-2.21 (m, 1H), 2.12- | |
| | | | | 1.91 (m, 1H), 1.66 (d, J = 6.8 Hz, | |
| | | | | 3H), 0.67 (d, J = 6.8 Hz, 3H). | |

Note
intermediates I-B14, I-B19 and I-B21 were all commercially available.

The diastereomers in the table were subjected to chiral HPLC analysis under the following conditions (flow rate: 1 mL/minute; detector: UV 254 nm)

| Compound | Column | Mobile phase | RT/minute | de % |
|---|---|---|---|---|
| 23 | IC (0.46 × 25 cm) | Ethanol (0.1% diethylamine) | 4.588 | 100% |
| 24 | | | 5.141 | 83.53% |
| 26 | IC (0.46 × 25 cm) | Ethanol (0.1% diethylamine) | 4.707 | 100% |
| 27 | | | 6.297 | 96.50% |
| 31 | IC (0.46 × 25 cm) | Ethanol (0.1% diethylamine) | 5.856 | 100% |
| 32 | | | 7.844 | 91.38% |
| 34 | IC (0.46 × 25 cm) | n-Heptane/ethanol (0.1% | 7.897 | 100% |
| 35 | | diethylamine) = 50:50 | 12.010 | 100% |
| 38 | IC (0.46 × 25 cm) | Ethanol (0.1% diethylamine) | 4.354 | 100% |
| 39 | | | 5.411 | 92.03% |
| 41 | IC (0.46 × 25 cm) | n-Heptane/ethanol (0.1% diethylamine) = 50:50 | 7.831 | 100% |
| 42 | | | 10.930 | 96.20% |
| 43 | IC (0.46 × 25 cm) | Ethanol (0.1% diethylamine) | 5.564 | 100% |
| 44 | | | 7.314 | 100% |
| 48 | IC (0.46 × 25 cm) | n-Heptane/ethanol (0.1% | 14.165 | 100% |
| 49 | | diethylamine) = 70:30 | 21.536 | 100% |
| 50 | IC (0.46 × 25 cm) | n-Heptane/ethanol (0.1% | 13.106 | 100% |
| 51 | | diethylamine) = 70:30 | 19.207 | 95.00% |
| 53 | OJH (0.46 × 15 cm) | Carbon dio×ide/ethanol | 3.393 | 100% |
| | | (0.05% diethylamine) = | | |
| 54 | | 60:40 | 7.447 | 98.68% |
| 55 | IC (0.46 × 25 cm) | Ethanol (0.1% diethylamine) | 5.019 | 100% |
| 56 | | | 6.141 | 100% |
| 57 | IC (0.46 × 25 cm) | n-Heptane/ethanol (0.1% | 9.398 | 74.74% |
| 58 | | diethylamine) = 70:30 | 12.837 | 89.01% |
| 61 | ODH (0.46 × 25 cm) | n-Heptane/ethanol (0.1% | 10.768 | 100% |
| 62 | | diethylamine) = 50:50 | 12.575 | 93.58% |
| 71 | ODH (0.46 × 25 cm) | n-Heptane/ethanol (0.1% | 9.172 | 100% |
| 72 | | diethylamine) = 50:50 | 11.299 | 97.59% |
| 74 | ODH (0.46 × 25 cm) | n-Heptane/ethanol (0.1% | 14.366 | 100% |
| 75 | | diethylamine) = 50:50 | 16.520 | 94.31% |
| 76 | IG (0.46 × 25 cm) | n-Heptane/ethanol (0.1% | 4.028 | 100% |
| 77 | | diethylamine) = 20:80 | 9.252 | 95.96% |
| 78 | IG (0.46 × 25 cm) | n-Heptane/ethanol (0.1% | 4.605 | 100% |
| 79 | | diethylamine) = 20:80 | 11.536 | 96.47% |
| 80 | IG (0.46 × 25 cm) | n-Heptane/ethanol (0.1% | 4.264 | 100% |
| 81 | | diethylamine) = 20:80 | 9.422 | 100% |
| 85 | ODH (0.46 × 25 cm) | n-Heptane/ethanol (0.1% | 8.204 | 100% |
| 86 | | diethylamine) = 50:50 | 9.798 | 97.99% |
| 88 | ODH (0.46 × 25 cm) | n-Heptane/ethanol (0.1% | 17.547 | 100% |
| 89 | | diethylamine) = 70:30 | 21.969 | 97.11% |
| 90 | IG (0.46 × 25 cm) | n-Heptane/ethanol (0.1% | 4.857 | 100% |
| 91 | | diethylamine) = 50:50 | 10.323 | 100% |
| 92 | IG (0.46 × 25 cm) | n-Heptane/ethanol (0.1% | 5.645 | 100% |
| 93 | | diethylamine) = 50:50 | 10.727 | 100% |
| 95 | IG (0.46 × 25 cm) | Acetonitrile/ethanol (0.1% | 2.648 | 100% |
| 96 | | diethylamine) = 10:90 | 4.316 | 100% |
| 98 | IG (0.46 × 25 cm) | Ethanol (0.1% diethylamine) | 3.179 | 100% |
| 99 | | | 5.943 | 100% |
| 100 | ODH (0.46 × 25 cm) | n-Heptane/ethanol (0.1% | 9.575 | 100% |
| 101 | | diethylamine) = 50:50 | 12.650 | 100% |
| 102 | IG (0.46 × 25 cm) | Acetonitrile/ethanol (0.1% | 2.815 | 100% |
| 103 | | diethylamine) = 10:90 | 4.644 | 100% |
| 109 | IG (0.46 × 25 cm) | n-Heptane/ethanol (0.1% | 4.596 | 100% |
| 110 | | diethylamine) = 20:80 | 11.619 | 100% |
| 115 | ODH (0.46 × 25 cm) | n-Heptane/ethanol (0.1% | 11.984 | 100% |
| 116 | | diethylamine) = 50:50 | 14.450 | 93.70 |
| 124 | ODH (0.46 × 25 cm) | n-Heptane/ethanol (0.1% | 6.345 | 100% |
| 125 | | diethylamine) = 20:80 | 8.005 | 98.45% |
| 127 | ODH (0.46 × 25 cm) | n-Heptane/ethanol (0.1% | 7.199 | 100% |
| 128 | | diethylamine) = 20:80 | 10.739 | 99.73% |
| 130 | IC (0.46 × 25 cm) | Ethanol (0.1% diethylamine) | 5.807 | 100% |
| 131 | | | 7.488 | 89.21% |
| 132 | IG (0.46 × 25 cm) | n-Heptane/ethanol (0.1% | 4.733 | 100% |
| 133 | | diethylamine) = 20:80 | 10.779 | 89.79 |
| 137 | ODH (0.46 × 25 cm) | n-Heptane/ethanol (0.1% | 6.388 | 100% |
| 138 | | diethylamine) = 20:80 | 8.186 | 98.22% |
| 142 | IC (0.46 × 25 cm) | Ethanol (0.1% diethylamine) | 5.712 | 100% |
| 143 | | | 7.577 | 100% |
| 144 | ODH (0.46 × 25 cm) | n-Heptane/ethanol (0.1% | 11.025 | 100% |
| 145 | | diethylamine) = 70:30 | 17.501 | 100% |
| 151 | IC (0.46 × 25 cm) | n-Heptane/ethanol (0.1% | 13.221 | 100% |
| 152 | | diethylamine) = 50:50 | 16.306 | 91.45% |
| 153 | ODH (0.46 × 25 cm) | n-Heptane/ethanol (0.1% | 11.983 | 100% |
| 154 | | diethylamine) = 50:50 | 14.917 | 100% |
| 160 | ODH (0.46 × 25 cm) | Ethanol (0.1% diethylamine) | 11.620 | 100% |
| 161 | | | 14.069 | 93.56% |
| 166 | ODH (0.46 × 25 cm) | n-Heptane/ethanol (0.1% | 5.858 | 97.36% |
| 167 | | diethylamine) = 20:80 | 7.140 | 99.03% |
| 183 | ODH (0.46 × 25 cm) | n-Heptane/ethanol (0.1% | 6.213 | 100% |
| 184 | | diethylamine) = 20:80 | 7.975 | 98.00% |
| 199 | ODH (0.46 × 25 cm) | n-Heptane/ethanol (0.1% | 6.394 | 100% |
| 200 | | diethylamine) = 20:80 | 7.775 | 100% |
| 202 | IG (0.46 × 25 cm) | n-Heptane/ethanol (0.1% | 4.529 | 100% |
| 203 | | diethylamine) = 20:80 | 10.636 | 100% |
| 206 | ODH (0.46 × 25 cm) | n-Heptane/ethanol (0.1% | 7.477 | 100% |
| 207 | | diethylamine) = 50:50 | 10.403 | 100% |
| 208 | ODH (0.46 × 25 cm) | n-Heptane/ethanol (0.1% | 9.532 | 100% |
| 209 | | diethylamine) = 50:50 | 11.673 | 100% |
| 210 | ODH (0.46 × 25 cm) | n-Heptane/ethanol (0.1% | 7.301 | 100% |
| 211 | | diethylamine) = 50:50 | 8.659 | 97.72% |
| 212 | ODH (0.46 × 25 cm) | n-Heptane/ethanol (0.1% | 8.483 | 100% |
| 213 | | diethylamine) = 50:50 | 13.086 | 96.74% |
| 214 | ODH (0.46 × 25 cm) | n-Heptane/ethanol (0.1% | 16.389 | 100% |
| 215 | | diethylamine) = 70:30 | 26.529 | 100% |
| 216 | ODH (0.46 × 25 cm) | n-Heptane/ethanol (0.1% | 16.620 | 100% |
| 217 | | diethylamine) = 70:30 | 23.144 | 100% |
| 219 | ODH (0.46 × 25 cm) | n-Heptane/ethanol (0.1% | 13.950 | 100% |
| 220 | | diethylamine) = 70:30 | 19.691 | 95.32% |
| 222 | ODH (0.46 × 25 cm) | n-Heptane/ethanol (0.1% | 7.512 | 100% |
| 223 | | diethylamine) = 50:50 | 10.597 | 87.99% |
| 247 | ODH (0.46 × 25 cm) | n-Heptane/ethanol (0.1% | 5.524 | 100% |
| 248 | | diethylamine) = 70:30 | 9.633 | 100% |
| 250 | ODH (0.46 × 25 cm) | n-Heptane/ethanol (0.1% | 5.078 | 100% |
| 263 | | diethylamine) = 50:50 | 4.376 | 100% |
| 251 | ODH (0.46 × 25 cm) | n-Heptane/ethanol (0.1% | 10.558 | 100% |
| 252 | | diethylamine) = 70:30 | 15.225 | 93.94% |
| 253 | ODH (0.46 × 25 cm) | n-Heptane/ethanol (0.1% | 6.095 | 100% |
| 254 | | diethylamine) = 70:30 | 8.844 | 100% |
| 255 | ODH (0.46 × 25 cm) | n-Heptane/ethanol (0.1% | 4.221 | 100% |
| 256 | | diethylamine) = 20:80 | 3.847 | 100% |
| 257 | IC (0.46 × 25 cm) | n-Heptane/ethanol (0.1% | 8.610 | 100% |
| 258 | | diethylamine) = 50:50 | 12.931 | 98.72% |
| 260 | ODH (0.46 × 25 cm) | n-Heptane/ethanol (0.1% | 9.426 | 100% |
| 261 | | diethylamine) = 70:30 | 16.690 | 100% |
| 264 | ODH (0.46 × 25 cm) | n-Heptane/ethanol (0.1% | 9.851 | 100% |
| 265 | | diethylamine) = 70:30 | 16.897 | 100% |
| 269 | ODH (0.46 × 25 cm) | n-Heptane/ethanol (0.1% | 5.256 | 100% |
| 272 | | diethylamine) = 50:50 | 4.436 | 100% |
| 270 | ODH (0.46 × 25 cm) | n-Heptane/ethanol (0.1% | 10.377 | 100% |
| 271 | | diethylamine) = 70:30 | 7.746 | 100% |
| 273 | ODH (0.46 × 25 cm) | n-Heptane/ethanol (0.1% | 6.365 | 100% |
| 274 | | diethylamine) = 70:30 | 5.604 | 100% |
| 275 | IC (0.46 × 25 cm) | n-Heptane/ethanol (0.1% | 5.297 | 100% |
| 276 | | diethylamine) = 50:50 | 5.611 | 100% |
| 277 | ODH (0.46 × 25 cm) | n-Heptane/ethanol (0.1% | 6.384 | 100% |
| 278 | | diethylamine) = 70:30 | 8.656 | 100% |
| 279 | ODH (0.46 × 25 cm) | n-Heptane/ethanol (0.1% | 7.096 | 97.68% |
| 280 | | diethylamine) = 70:30 | 5.821 | 91.04% |
| 282 | ODH (0.46 × 25 cm) | n-Heptane/ethanol (0.1% | 15.022 | 100% |
| 283 | | diethylamine) = 70:30 | 9.785 | 100% |
| 284 | ODH (0.46 × 25 cm) | n-Heptane/ethanol (0.1% | 7.327 | 100% |
| 285 | | diethylamine) = 70:30 | 6.202 | 100% |
| 286 | ODH (0.46 × 25 cm) | n-Heptane/ethanol (0.1% | 10.101 | 100% |
| 287 | | diethylamine) = 70:30 | 16.915 | 100% |

The enantiomers in the table were subjected to chiral HPLC analysis under the following conditions (flow rate: 1 mL/minute; detector: UV 254 nm):

| Com- | | | RT/ | |
|---|---|---|---|---|
| pound | Column | Mobile phase | minute | ee % |
| 29 | IC (0.46 × | n-Heptane/ethanol (0.1% | 8.728 | 100% |
| 30 | 25 cm) | diethylamine) = 50:50 | 11.370 | 94.31% |
| 117 | IG (0.46 × | n-Heptane/ethanol (0.1% | 6.061 | 100% |
| 118 | 25 cm) | diethylamine) = 20:80 | 20.437 | 100% |

### Compound 28 1-(1O-(1-(2-Aminopyridin-3-yl)ethyl)-4-fluoro-2-(((S)-1-methylpyrrolidin-2-yl)methoxy)-9,10-dihydro-8H-7-oxa-1,3,6,10-tetraazacyclohepta[de]naphthalen-5-yl)isoquinolin-3-amine

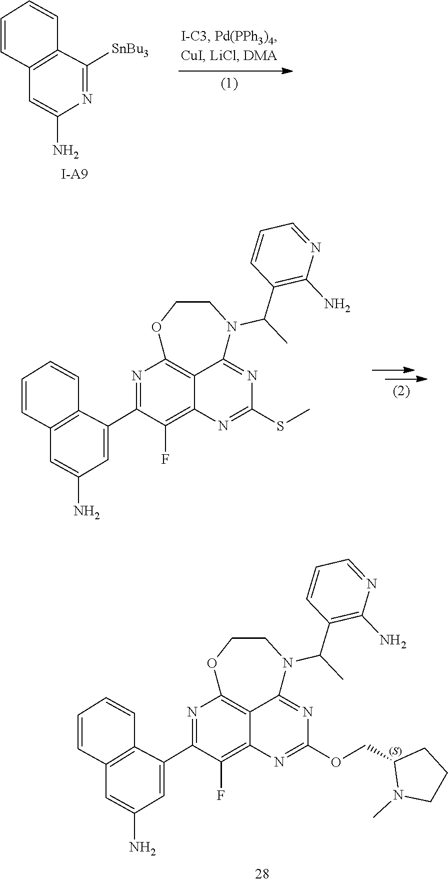

### Step 1: 1-(10-(1-(2-aminopyridin-3-yl)ethyl)-4-fluoro-2-(methylthio)-9,10-dihydro-8H-7-oxa-1,3,6,10-tetraazacyclohepta[de]naphthalen-5-yl)isoquinolin-3-amine

Crude intermediate I-A9 (0.9 mmol), the intermediate I-C3 (200 mg, 0.5 mmol), Pd(PPh₃)₄(58 mg, 0.05 mmol), cuprous iodide (29 mg, 0.15 mmol) and lithium chloride (84 mg, 2 mmol) were added in DMA (8 mL) and stirred at 120° C. for 2 hours under nitrogen protection. The resulting product was purified by column chromatography on silica gel (methanol/water) and preparative thin-layer chromatography (dichloromethane/methanol) to give the target product (80 mg, yield 31%) as a yellow solid. [M+H]⁺ 515.2

### Step 2: 1-(10-(1-(2-aminopyridin-3-yl)ethyl)-4-fluoro-2-(((S)-1-methylpyrrolidin-2-yl)methoxy)-9,10-dihydro-8H-7-oxa-1,3,6,10-tetraazacyclohepta[de]naphthalen-5-yl)isoquinolin-3-amine

The target compound 28 was prepared from corresponding intermediates and reagents by following the preparation steps 2 and 3 of the compound 2. [M+H]⁺ 582.3. ¹H NMR (400 MHz, CD₃OD) δ 7.94 (d, J=5.0 Hz, 1H), 7.74 (d, J=7.5 Hz, 1H), 7.64-7.44 (m, 3H), 7.20-7.10 (m, 1H), 6.92 (s, 1H), 6.82-6.73 (m, 1H), 6.66-6.54 (m, 1H), 4.60-4.41 (m, 3H), 4.40-4.30 (m, 1H), 3.80-3.72 (m, 1H), 3.61-3.46 (m, 1H), 3.15-3.00 (m, 1H), 2.85-2.81 (m, 1H), 2.51 (s, 3H), 2.40-2.32 (m, 1H), 2.15-2.05 (m, 1H), 1.90-1.71 (m, 3H), 1.63 (d, J=10.2 Hz, 3H).

The compounds in the table below were prepared from corresponding intermediates and reagents by following the preparation steps of the compound 28:

| | | LC-MS | |
|---|---|---|---|
| Compound | Structural formula | [M + H]⁺ | ¹HNMR |
| 329 | 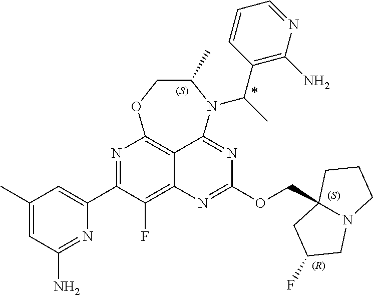 | 604.3 | (400 MHz, CD₃OD) δ 7.97-7.91 (m, 1H), 7.79-7.75 (m, 1H), 7.03-7.00 (m, 1H), 6.78-6.68 (m, 2H), 6.50-6.46 (m, 1H), 5.37-5.20 (m, 1H), 4.63-4.57 (m, 1H), 4.35-4.24 (m, 3H), 4.08-3.99 (m, 1H), 3.28-3.11 (m, 3H), 3.03-2.95 (m, 1H), 2.38-2.12 (m, 6H), 2.02-1.85 (m, 3H), 1.67-1.60 (m, 3H), 0.69-0.64 (m, 3H). |

### Compound 326 (*)6-((9S)-10-(1-(2-aminopyridin-3-yl)ethyl)-4-fluoro-2-(((2R,7aS)-2-fluorotetrahydro-1H-pyrrolizin-7a(5H)-yl)methoxy)-9-methyl-9,10-dihydro-8H-7-oxa-1,3,6,10-tetraazacyclohepta[de]naphthalen-5-yl)-4-methyl-5-(trifluoromethyl)pyridin-2-amine

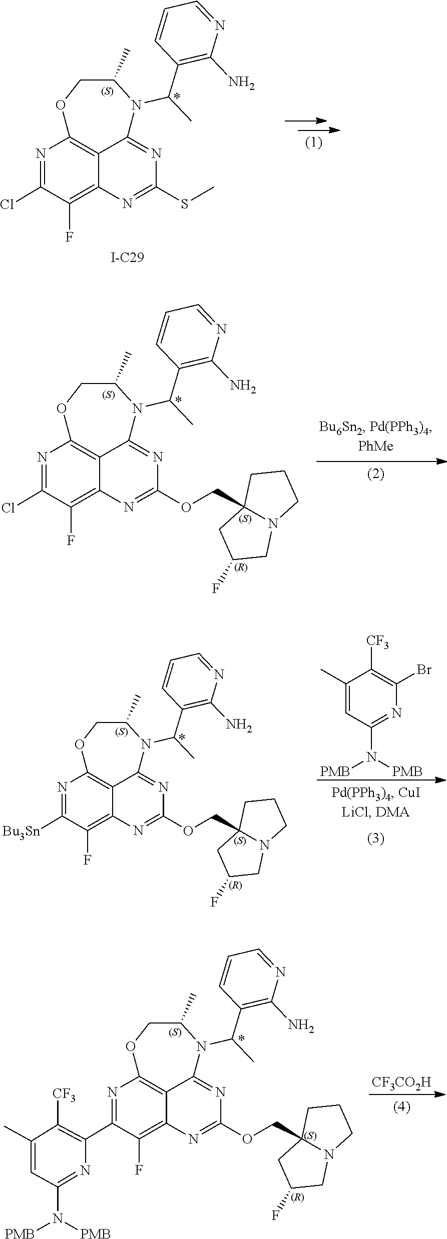

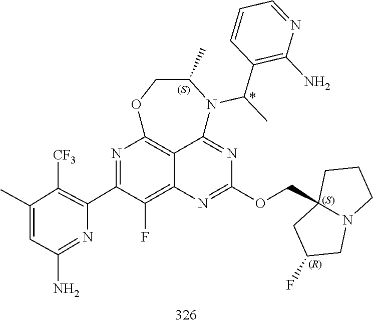

Step 1: (*)3-(1-((S)-5-chloro-4-fluoro-2-(((2R,7aS)-2-fluorotetrahydro-1H-pyrrolizin-7a(5H)-yl)methoxy)-9-methyl-8,9-dihydro-10OH-7-oxa-1,3,6,10-tetraazacyclohepta[de]naphthalen-10-yl)ethyl)pyridin-2-amine

The target intermediate was prepared from corresponding intermediates and reagents by following the preparation steps 1 and 2 of the compound 1. [M+H]⁺ 532.2

### Step 2: (*)3-(1-((S)-4-fluoro-2-(((2R,7aS)-2-fluorotetrahydro-1H-pyrrolizin-7a(5H)-yl)methoxy)-9-methyl-5-(tributylstannyl)-8,9-dihydro-1OH-7-oxa-1,3,6,10-tetraazacyclohepta[de]naphthalen-10-yl)ethyl)pyridin-2-amine

(*)3-(1-((S)-5-chloro-4-fluoro-2-(((2R,7aS)-2-fluorotetrahydro-1H-pyrrolizin-7a(5H)-yl)methoxy)-9-methyl-8,9-dihydro-1OH-7-oxa-1,3,6,10-tetraazacyclohepta[de]naphthalen-10-yl)ethyl)pyridin-2-amine (500 mg, 0.94 mmol), hexa-n-butylditin (1.09 g, 1.88 mmol) and Pd(PPh₃)₄(217 mg, 0.188 mmol) were added in toluene (6 mL) and stirred at 100° C. for 16 hours under nitrogen protection. The reaction solution was concentrated under reduced pressure and purified by column chromatography on silica gel (dichloromethane/methanol) to give the target product (300 mg, yield 41%) as a yellow solid. [M+H]⁺ 788.4

### Step 3: (*)6-((9S)-10-(1-(2-aminopyridin-3-yl)ethyl)-4-fluoro-2-(((2R,7aS)-2-fluorotetrahydro-1H-pyrrolizin-7a(5H)-yl)methoxy)-9-methyl-9,10-dihydro-8H-7-oxa-1,3,6,10-tetraazacyclohepta[de]naphthalen-5-yl)-N,N-bis(4-methoxybenzyl)-4-methyl-5-(trifluoromethyl)pyridin-2-amine

The target intermediate was prepared from corresponding intermediates and reagents by following the preparation step 1 of the compound 28. [M+H]⁺ 912.4

### Step 4: (*)6-((9S)-10-(1-(2-aminopyridin-3-yl)ethyl)-4-fluoro-2-(((2R,7aS)-2-fluorotetrahydro-1H-pyrrolizin-7a(5H)-yl)methoxy)-9-methyl-9,10-dihydro-8H-7-oxa-1,3,6,10-tetraazacyclohepta[de]naphthalen-5-yl)-4-methyl-5-(trifluoromethyl)pyridin-2-amine

(*)6-((9)1O-(1-(2-aminopyridin-3-yl)ethyl)-4-fluoro-2-(((2R, 7aS)-2-fluorotetrahydro-1H-pyrrolizin-7a(5H)-yl)methoxy)-9-methyl-9,10-dihydro-8H-7-oxa-1,3,6,10-tetraazacyclohepta[de]naphthalen-5-yl)-N,N-bis(4-methoxybenzyl)-4-methyl-5-(trifluoromethyl)pyridin-2-amine (20 mg, 0.022 mmol) was added in trifluoroacetic acid (3 mL) and stirred at 55° C. for 2 hours under nitrogen protection. The reaction solution was concentrated under reduced pressure and purified by column chromatography on silica gel (methanol/water) and preparative thin-layer chromatography (dichloromethane/methanol) to give the target product (10 mg, yield 68% o) as a yellow solid. [M+H]⁺ 672.2. ¹H NMR (400 MHz, CD₃OD) δ 7.99-7.94 (m, 1H), 7.83-7.76 (m, 1H), 6.81-6.71 (m, 2H), 6.60-6.54 (m, 1H), 5.39-5.18 (m, 1H), 4.64-4.56 (m, 1H), 4.36-4.25 (m, 3H), 4.13-3.99 (m, 1H), 3.26-3.19 (m, 2H), 3.17-3.12 (m, 1H), 3.03-2.95 (m, 1H), 2.45-2.39 (m, 3H), 2.37-2.13 (m, 3H), 2.04-1.88 (m, 3H), 1.66 (d, J=6.8 Hz, 3H), 0.67 (d, J=6.9 Hz, 3H).

The compounds in the table below were prepared from corresponding intermediates and reagents by following the preparation steps of the compound 326:

| | | LC-MS | |
|---|---|---|---|
| Compound | Structural formula | [M + H]⁺ | ¹HNMR |
| 330 | 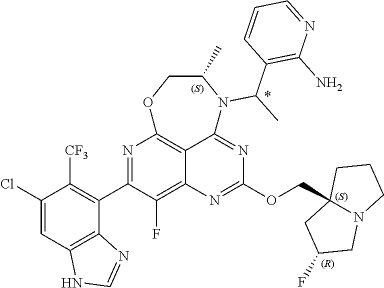 | 716.2 | (400 MHz, CD₃OD) δ 8.01-7.95 (m, 1H), 7.96-7.92 (m, 1H), 7.82 (s, 1H), 7.81-7.67 (m, 1H), 6.84-6.68 (m, 2H), 5.47-5.22 (m, 1H), 4.67-4.58 (m, 1H), 4.42-4.30 (m, 3H), 4.15-4.03 (m, 1H), 3.43-3.31 (m, 3H), 3.12- 3.04 (m, 1H), 2.43-2.13 (m, 3H), 2.10-1.85 (m, 3H), 1.69 (d, J = 6.9 Hz, 3H), 0.78-0.68 (m, 3H). |
| 335 | 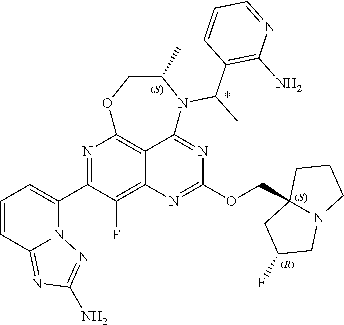 | 630.2 | (400 MHz, CD₃OD) δ 7.99-7.93 (m, 1H), 7.82-7.75 (m, 1H), 7.67-7.59 (m, 1H), 7.52- 7.46 (m, 1H), 7.21-7.13 (m, 1H), 6.81-6.68 (m, 2H), 5.41-5.18 (m, 1H), 4.68-4.56 (m, 1H), 4.41-4.24 (m, 3H), 4.15-4.01 (m, 1H), 3.26-3.13 (m, 3H), 3.05-2.94 (m, 1H), 2.39- 2.12 (m, 3H), 2.04-1.87 (m, 3H), 1.72-1.63 (m, 3H), 0.74-0.65 (m, 3H). |
| 362 | 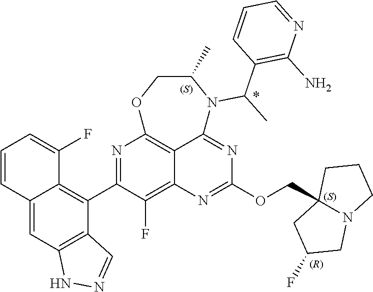 | 682.4 | (400 MHz, CD₃OD) δ 8.24-8.20 (m, 1H), 8.09-7.92 (m, 2H), 7.90-7.85 (m, 1H), 7.84- 7.78 (m, 1H), 7.44-7.35 (m, 1H), 7.09-6.98 (m, 1H), 6.85-6.75 (m, 2H), 5.43-5.18 (m, 1H), 4.69-4.57 (m, 1H), 4.40-4.28 (m, 3H), 4.14-4.06 (m, 1H), 3.28-3.15 (m, 3H), 3.05- 2.96 (m, 1H), 2.41-2.14 (m, 3H), 2.03-1.88 (m, 3H), 1.74-1.66 (m, 3H), 0.83-0.73 (m, 3H). |
| 363 | 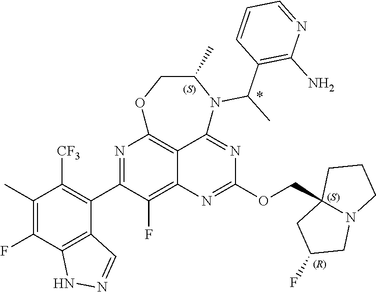 | 714.4 | (400 MHz, CD₃OD) δ 8.03-7.93 (m, 1H), 7.85-7.68 (m, 2H), 6.81- 6.73 (m, 2H), 5.43- 5.20 (m, 1H), 4.70-4.59 (m, 1H), 4.40-4.29 (m, 3H), 4.17-4.04 (m, 1H), 3.29-3.16 (m, 3H), 3.10-2.98 (m, 1H), 2.61-2.52 (m, 3H), 2.43-2.13 (m, 3H), 2.07-1.88 (m, 3H), 1.73- 1.64 (m, 3H), 0.79-0.69 (m, 3H). |
| 348 | 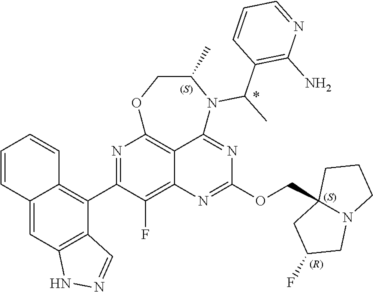 | 664.4 | (400 MHz, CD₃OD) δ 8.22-7.96 (m, 4H), 7.94-7.75 (m, 2H), 7.51-7.41 (m, 1H), 7.41- 7.29 (m, 1H), 6.87-6.74 (m, 2H), 5.41-5.19 (m, 1H), 4.72-4.59 (m, 1H), 4.45-4.27 (m, 3H), 4.17-4.05 (m, 1H), 3.28-3.15 (m, 3H), 3.09-2.95 (m, 1H), 2.41-2.13 (m, 3H), 2.05- 1.89 (m, 3H), 1.77-1.64 (m, 3H), 0.82-0.72 (m, 3H). |
| 343 | 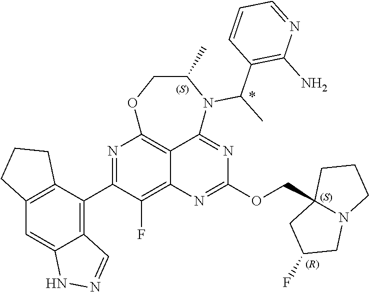 | 654.4 | (400 MHz, CD₃OD) δ 7.99-7.94 (m, 1H), 7.85-7.74 (m, 2H), 7.48-7.43 (m, 1H), 6.83- 6.71 (m, 2H), 5.40-5.19 (m, 1H), 4.67-4.57 (m, 1H), 4.39-4.28 (m, 3H), 4.14-3.99 (m, 1H), 3.27-3.15 (m, 3H), 3.08-2.83 (m, 5H), 2.39-2.08 (m, 5H), 2.03-1.89 (m, 3H), 1.71- 1.63 (m, 3H), 0.78-0.68 (m, 3H). |
| 367 | 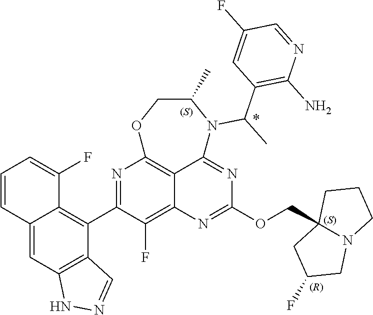 | 699.7 | (400 MHz, CD₃OD) δ 8.25-8.20 (m, 1H), 8.08-7.84 (m, 3H), 7.75-7.67 (m, 1H), 7.46- 7.36 (m, 1H), 7.09-6.97 (m, 1H), 6.85-6.75 (m, 1H), 5.40-5.20 (m, 1H), 4.69-4.60 (m, 1H), 4.43-4.28 (m, 3H), 4.17-4.08 (m, 1H), 3.28-3.14 (m, 3H), 3.06-2.95 (m, 1H), 2.28- 2.13 (m, 3H), 2.03-1.96 (m, 3H), 1.73-1.66 (m, 3H), 0.83-0.76 (m, 3H). |
| 368 | 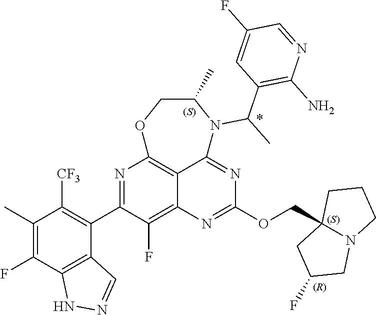 | 731.7 | (400 MHz, CD₃OD) δ 7.94-7.88 (m, 1H), 7.84-7.66 (m, 2H), 6.81-6.73 (m, 1H), 5.40- 5.20 (m, 1H), 4.67-4.59 (m, 1H), 4.41-4.27 (m, 3H), 4.16-4.07 (m, 1H), 3.28-3.14 (m, 3H), 3.06-2.97 (m, 1H), 2.60-2.53 (m, 3H), 2.39-2.12 (m, 3H), 2.03-1.88 (m, 3H), 1.71- 1.65 (m, 3H), 0.80-0.72 (m, 3H). |
| 369 | 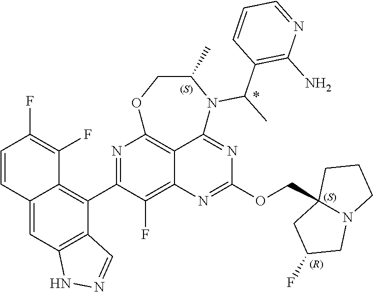 | 700.7 | (400 MHz, CD₃OD) δ 8.24 (d, J = 1.8 Hz, 1H), 8.12-7.95 (m, 2H), 7.93-7.87 (m, 1H), 7.83-7.79 (m, 1H), 7.48-7.36 (m, 1H), 6.83- 6.72 (m, 2H), 5.41-5.19 (m, 1H), 4.70-4.59 (m, 1H), 4.41-4.30 (m, 3H), 4.15-4.07 (m, 1H), 3.27-3.15 (m, 3H), 3.05-2.95 (m, 1H), 2.40-2.14 (m, 3H), 2.03-1.90 (m, 3H), 1.69 (d, J = 6.8 Hz, 3H), 0.83-0.74 (m, 3H). |
| 370 | 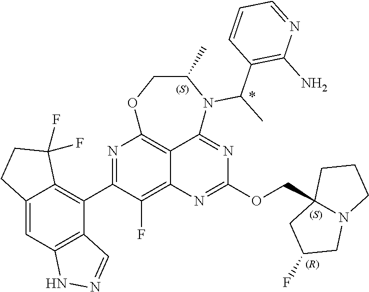 | 690.4 | (400 MHz, CD₃OD) δ 8.00-7.89 (m, 1H), 7.91-7.66 (m, 2H), 7.53 (s, 1H), 6.83-6.67 (m, 2H), 5.39-5.16 (m, 1H), 4.66-4.55 (m, 1H), 4.40-4.22 (m, 3H), 4.10-3.98 (m, 1H), 3.28-3.12 (m, 5H), 3.04-2.92 (m, 1H), 2.71- 2.49 (m, 2H), 2.39-2.11 (m, 3H), 2.05-1.81 (m, 3H), 1.65 (d, J = 6.8 Hz, 3H), 0.71 (d, J = 6.8 Hz, 3H). |
| 371 | 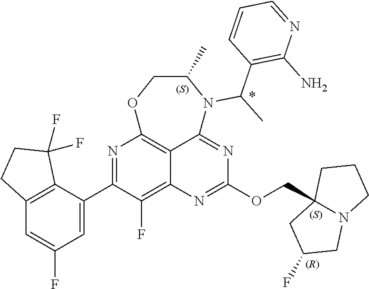 | 668.4 | (400 MHz, CD₃OD) δ 8.03-7.81 (m, 1H), 7.84-7.71 (m, 1H), 7.26-7.17 (m, 1H), 7.18- 7.05 (m, 1H), 6.82-6.68 (m, 2H), 5.41-5.19 (m, 1H), 4.65-4.56 (m, 1H), 4.35-4.24 (m, 3H), 4.11- 4.00 (m, 1H), 3.27-2.96 (m, 6H), 2.64-2.49 (m, 2H), 2.36 -2.10 (m, 3H), 2.03- 1.86 (m, 3H), 1.66 (d, J = 6.8 Hz, 3H), 0.68 (d, J = 6.9 Hz, 3H). |
| 372 | 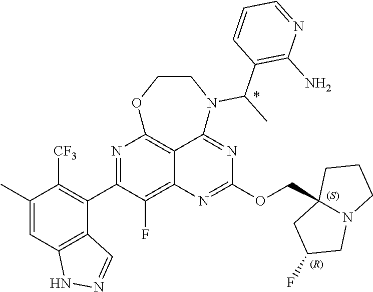 | 682.2 | (400 MHz, CD₃OD) δ 7.97-7.90 (m, 1H), 7.76-7.69 (m, 1H), 7.69-7.60 (m, 2H), 6.78- 6.71 (m, 1H), 6.65-6.55 (m, 1H), 5.38-5.17 (m, 1H), 4.51-4.43 (m, 1H), 4.41-4.27 (m, 3H), 3.84-3.71 (m, 1H), 3.62-3.48 (m, 1H), 3.27-3.11 (m, 3H), 3.05-2.92 (m, 1H), 2.70- 2.61 (m, 3H), 2.41-2.10 (m, 3H), 2.05-1.85 (m, 3H), 1.70-1.57 (m, 3H). |
| 373 (by- product of 370) | 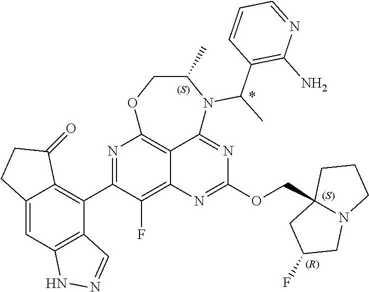 | 667.7 | (400 MHz, CD₃OD) δ 8.10-7.89 (m, 2H), 7.84-7.76 (m, 1H), 7.69 (s, 1H), 6.81-6.73 (m, 2H), 5.41-5.19 (m, 1H), 4.67-4.60 (m, 1H), 4.41-4.25 (m, 3H), 4.14-4.01 (m, 1H), 3.28-3.17 (m, 5H), 3.08-2.96 (m, 1H), 2.83- 2.69 (m, 2H), 2.40-2.11 (m, 3H), 2.06-1.89 (m, 3H), 1.74-1.63 (m, 3H), 0.79-0.68 (m, 3H). |
| 374 (by- product of 370) | 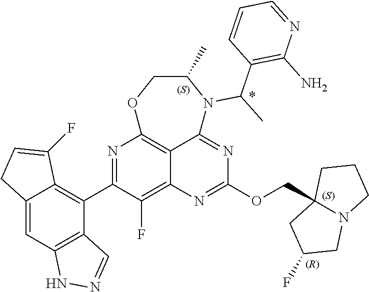 | 669.7 | (400 MHz, CD₃OD) δ 8.03-7.83 (m, 2H), 7.83-7.77 (m, 1H), 7.71 (s, 1H), 6.81-6.73 (m, 2H), 5.93-5.86 (m, 1H), 5.40-5.21 (m, 1H), 4.68-4.60 (m, 1H), 4.39-4.29 (m, 3H), 4.12-4.03 (m, 1H), 3.51-3.43 (m, 2H), 3.27- 3.15 (m, 3H), 3.07-2.98 (m, 1H), 2.37-2.13 (m, 3H), 2.05-1.92 (m, 3H), 1.72-1.64 (m, 3H), 0.78-0.68 (m, 3H). |
| 375 | 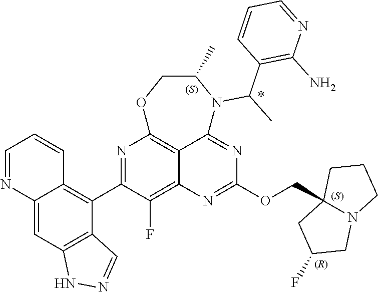 | 664.8 | (400 MHz, CD₃OD) δ 8.94-8.83 (m, 1H), 8.57-8.07 (m, 3H), 8.02-7.94 (m, 1H), 7.83- 7.74 (m, 1H), 7.46-7.36 (m, 1H), 6.83-6.73 (m, 2H), 5.40-5.17 (m, 1H), 4.71-4.60 (m, 1H), 4.46-4.26 (m, 3H), 4.16-4.01 (m, 1H), 3.26-3.12 (m, 3H), 3.06-2.91 (m, 1H), 2.39- 2.12 (m, 3H), 2.02-1.87 (m, 3H), 1.68 (d, J = 6.8 Hz, 3H), 0.75 (d, J = 6.9 Hz, 3H). |
| 376 | 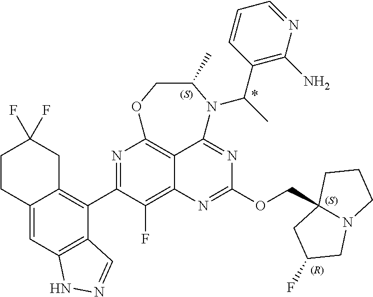 | 704.4 | (400 MHz, CD₃OD) δ 7.99-7.79 (m, 2H), 7.78-7.60 (m, 1H), 7.55-7.47 (m, 1H), 6.84- 6.71 (m, 2H), 5.39-5.21 (m, 1H), 4.66-4.60 (m, 1H), 4.40-4.27 (m, 3H), 4.14-4.03 (m, 1H), 3.27-2.97 (m, 8H), 2.41-2.11 (m, 5H), 2.04-1.87 (m, 3H), 1.75-1.62 (m, 3H), 0.80- 0.67 (m, 3H). |
| 377 | 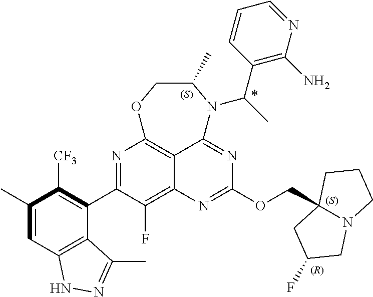 | 710.2 | (400 MHz, CD₃OD) δ 8.00-7.94 (m, 1H), 7.83-7.78 (m, 1H), 7.58-7.54 (m, 1H), 6.80- 6.73 (m, 2H), 5.38-5.20 (m, 1H), 4.67-4.60 (m, 1H), 4.42-4.26 (m, 3H), 4.14-4.04 (m, 1H), 3.27-3.14 (m, 3H), 3.05-2.95 (m, 1H), 2.71-2.61 (m, 3H), 2.39-2.12 (m, 3H), 2.02- 1.88 (m, 3H), 1.76 (s, 3H), 1.70-1.65 (m, 3H), 0.72-0.67 (m, 3H). |
| | + | | |
| 378 | 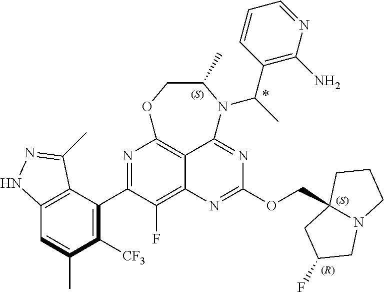 | 710.2 | (400 MHz, CD₃OD) δ 8.00-7.95 (m, 1H), 7.83-7.78 (m, 1H), 7.58-7.52 (m, 1H), 6.80- 6.72 (m, 2H), 5.41-5.22 (m, 1H), 4.66-4.60 (m, 1H), 4.40-4.31 (m, 3H), 4.13-4.05 (m, 1H), 3.33-3.30 (m, 1H), 3.28-3.21 (m, 2H), 3.08-3.00 (m, 1H), 2.67-2.59 (m, 3H), 2.42- 2.15 (m, 3H), 2.07-1.94 (m, 3H), 1.90 (s, 3H), 1.71-1.66 (m, 3H), 0.73-0.68 (m, 3H). |
| | 377 & 378 | | |
| 379 | 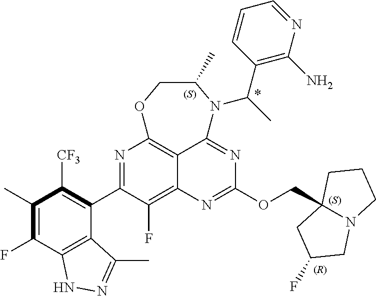 | 727.7 | (400 MHz, CD₃OD) δ 8.00-7.94 (m, 1H), 7.84-7.78 (m, 1H), 6.82-6.71 (m, 2H), 5.41- 5.21 (m, 1H), 4.66-4.60 (m, 1H), 4.43-4.29 (m, 3H), 4.15-4.04 (m, 1H), 3.28-3.17 (m, 3H), 3.07-2.98 (m, 1H), 2.57-2.47 (m, 3H), 2.41-2.13 (m, 3H), 2.05-1.86 (m, 6H), 1.73- 1.64 (m, 3H), 0.74-0.66 (m, 3H). |
| | + | | |
| 380 | 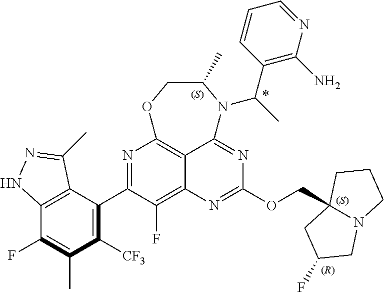 | 727.7 | (400 MHz, CD₃OD) δ 8.00-7.93 (m, 1H), 7.84-7.78 (m, 1H), 6.82-6.71 (m, 2H), 5.40- 5.20 (m, 1H), 4.67-4.61 (m, 1H), 4.44-4.27 (m, 3H), 4.15-4.04 (m, 1H), 3.28-3.17 (m, 3H), 3.07-2.97 (m, 1H), 2.54 (s, 3H), 2.41- 2.13 (m, 3H), 2.05-1.86 (m, 3H), 1.77 (s, 3H), 1.72-1.64 (m, 3H), 0.76-0.66 (m, 3H). |
| | 379 & 380 | | |
| 381 | 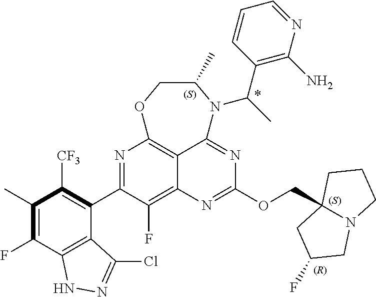 | 749.6 | (400 MHz, CD₃OD) δ 8.07-7.89 (m, 1H), 7.88-7.70 (m, 1H), 6.89-6.62 (m, 2H), 5.56- 5.07 (m, 1H), 4.68-4.59 (m, 1H), 4.45-4.21 (m, 3H), 4.17-3.95 (m, 1H), 3.28-3.10 (m, 3H), 3.07-2.92 (m, 1H), 2.59-2.47 (m, 3H), 2.40-2.08 (m, 3H), 2.02-1.84 (m, 3H), 1.72- 1.62 (m, 3H), 0.80-0.58 (m, 3H). |
| | + | | |
| 382 | 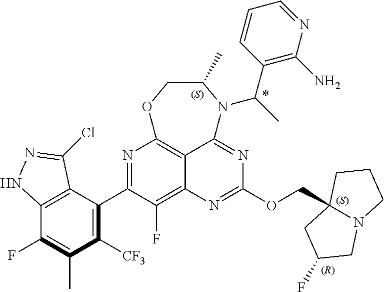 | 749.6 | (400 MHz, CD₃OD) δ 8.01-7.91 (m, 1H), 7.85-7.74 (m, 1H), 6.85-6.66 (m, 2H), 5.44- 5.15 (m, 1H), 4.67-4.58 (m, 1H), 4.45-4.27 (m, 3H), 4.15-4.01 (m, 1H), 3.28-3.11 (m, 3H), 3.11-2.92 (m, 1H), 2.65-2.49 (m, 3H), 2.45-2.10 (m, 3H), 2.05-1.82 (m, 3H), 1.75- 1.60 (m, 3H), 0.79-0.60 (m, 3H). |
| | 381 & 382 | | |
| 385 | 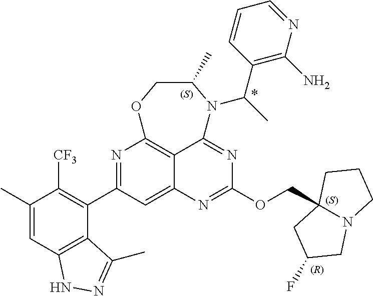 | 691.8 | (400 MHz, CD₃OD) δ 8.01-7.91 (m, 1H), 7.83-7.76 (m, 1H), 7.54-7.49 (m, 1H), 7.07- 7.01(m, 1H), 6.81-6.72 (m, 2H), 5.41-5.18 (m, 1H), 4.72-4.61 (m, 1H), 4.46-4.36 (m, 1H), 4.33-4.20 (m, 2H), 4.14-4.04 (m, 1H), 3.26-3.12 (m, 3H), 3.05-2.96 (m, 1H), 2.71- 2.60 (m, 3H), 2.37-2.12 (m, 3H), 2.04-1.91 (m, 3H), 1.90-1.72 (m, 3H), 1.71-1.64 (m, 3H), 0.76-0.66 (m, 3H). |
| 383 | 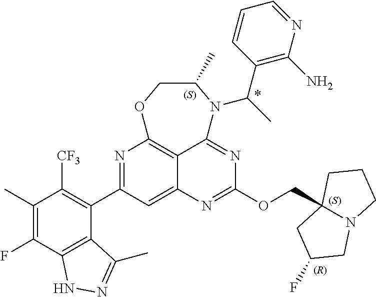 | 709.8 | (400 MHz, CD₃OD) δ 8.00-7.91 (m, 1H), 7.83-7.77 (m, 1H), 7.09-7.03 (m, 1H), 6.82- 6.71 (m, 2H), 5.40-5.18 (m, 1H), 4.72-4.62 (m, 1H), 4.46-4.37 (m, 1H), 4.34-4.20 (m, 2H), 4.15-4.02 (m, 1H), 3.26-3.13 (m, 3H), 3.06-2.94 (m, 1H), 2.55-2.46 (m, 3H), 2.37- 2.13 (m, 3H), 2.04-1.92 (m, 3H), 1.90-1.74 (m, 3H), 1.71-1.64 (m, 3H), 0.76-0.63 (m, 3H). |
| 386 | 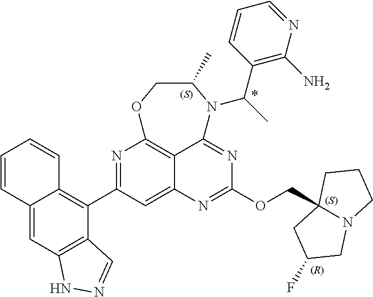 | 645.8 | (400 MHz, CD₃OD) δ 8.22-8.03 (m, 3H), 7.98-7.91 (m, 2H), 7.77-7.71 (m, 1H), 7.45- 7.37 (m, 2H), 7.33-7.26 (m, 1H), 6.83- 6.62 (m, 2H), 5.40-5.16 (m, 1H), 4.72-4.59 (m, 1H), 4.38-4.29 (m, 1H), 4.28-4.17 (m, 2H), 4.10-3.97 (m, 1H), 3.27-3.10 (m, 3H), 3.03- 2.91 (m, 1H), 2.37-2.07 (m, 3H), 1.99-1.83 (m, 3H), 1.68-1.58 (m, 3H), 0.79-0.69 (m, 3H). |
| 401 | 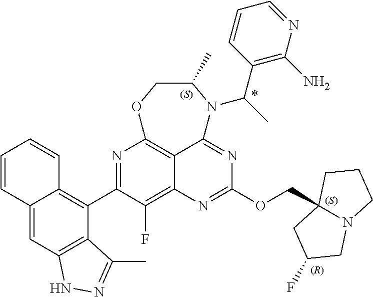 | 677.8 | (400 MHz, CD₃OD) δ 8.06 (s, 1H), 8.04- 7.92 (m, 2H), 7.87-7.77 (m, 1H), 7.74-7.50 (m, 1H), 7.49-7.38 (m, 1H), 7.36-7.21 (m, 1H), 6.84-6.71 (m, 2H), 5.44-5.15 (m, 1H), 4.67-4.61 (m, 1H), 4.48-4.25 (m, 3H), 4.21- 3.99 (m, 1H), 3.27-3.10 (m, 3H), 3.08-2.91 (m, 1H), 2.46-2.05 (m, 6H), 2.04-1.84 (m, 3H), 1.79-1.56 (m, 3H), 0.86-0.64 (m, 3H). |
| 402 | 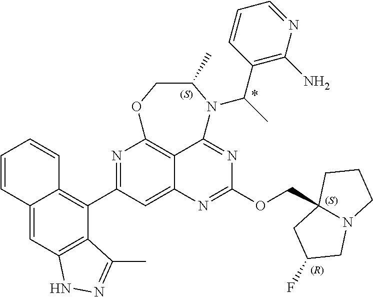 | 659.8 | (400 MHz, CD₃OD) δ 8.00 (s, 1H), 7.99- 7.92 (m, 2H), 7.84-7.57 (m, 2H), 7.47-7.35 (m, 1H), 7.33-7.17 (m, 2H), 6.88-6.71 (m, 2H), 5.41-5.11 (m, 1H), 4.71-4.63 (m, 1H), 4.49-4.38 (m, 1H), 4.36-4.20 (m, 2H), 4.20- 4.04 (m, 1H), 3.26-3.11 (m, 3H), 3.08-2.93 (m, 1H), 2.42-1.83 (m, 9H), 1.78-1.58 (m, 3H), 0.89-0.62 (m, 3H). |
| 403 | 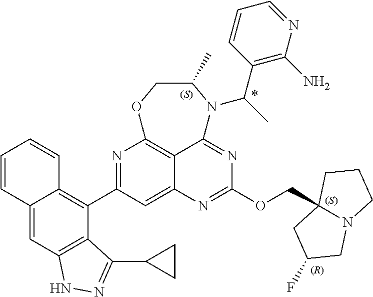 | 685.8 | (400 MHz, CD₃OD) δ 8.04-7.93 (m, 3H), 7.86-7.57 (m, 2H), 7.48-7.38 (m, 1H), 7.35- 7.20 (m, 2H), 6.87-6.76 (m, 2H), 5.41-5.22 (m, 1H), 4.69 (dd, J = 12.7, 5.4 Hz, 1H), 4.47-4.38 (m, 1H), 4.36-4.24 (m, 2H), 4.16- 4.07 (m, 1H), 3.22 (dd, J = 23.0, 9.1 Hz, 3H), 3.02 (d, J = 6.0 Hz, 1H), 2.41-2.14 (m, 3H), 2.05-1.89 (m, 3H), 1.72 (d, J = 6.9 Hz, 3H), 1.62-1.48 (m, 1H), 0.98-0.87 (m, 1H), 0.80-0.69 (m, 4H), 0.63-0.37 (m, 2H). |

The diastereomers in the table were obtained by separation through preparative thin-layer chromatography (dichloromethane/methanol/33% ammonia water=100/8/0.75). The HPLC analysis was performed under the following conditions (flow rate: 1 mL/minute; detector: UV 254 nm):

| Compound | Column | Mobile phase | RT/minute |
|---|---|---|---|
| 377 | YMC AQC18 | Methanol/water (0.1% formic | 7.125 |
| 378 | (0.46 × 15 cm) | acid) = 5%-95% (10 minutes) | 6.785 |
| 379 | YMC AQC18 | Methanol/water (0.1% formic | 7.062 |
| 380 | (0.46 × 15 cm) | acid) = 5%-95% (10 minutes) | 7.494 |
| 381 | YMC AQC18 | Methanol/water (0.1% formic | 7.087 |
| 382 | (0.46 × 15 cm) | acid) = 5%-95% (10 minutes) | 7.320 |

### Compound 336 (*)1-((9S)-10-(1-(2-aminopyridin-3-yl)ethyl)-4-fluoro-2-(((2R,7aS)-2-fluorotetrahydro-1H-pyrrolizin-7a(5H)-yl)methoxy)-9-methyl-9,10-dihydro-8H-7-oxa-1,3,6,10-tetraazacyclohepta[de]naphthalen-5-yl)-1H-indazol-3-amine

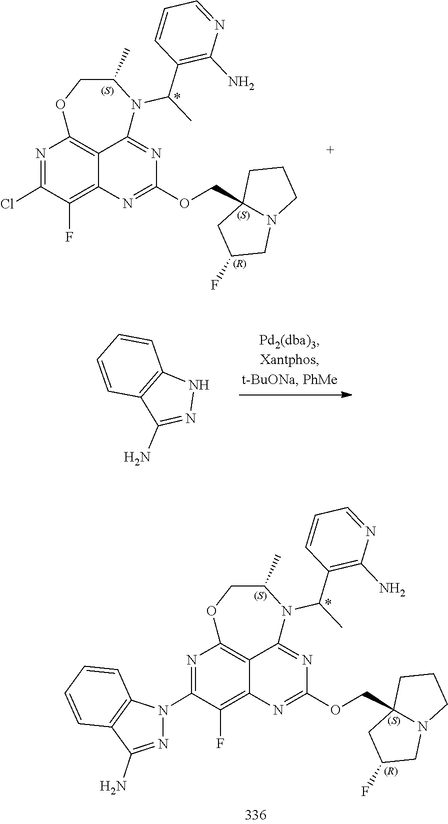

(*)3-(1-((S)-5-chloro-4-fluoro-2-(((2R,7aS)-2-fluorotetrahydro-H-pyrrolizin-7a(5H)-yl)methoxy)-9-methyl-8,9-dihydro-1OH-7-oxa-1,3,6,10-tetraazacyclohepta[de]naphthalen-10-yl)ethyl)pyridin-2-amine (100 mg, 0.19 mmol; the target intermediate prepared in step 1 of the compound 326), 1H-indazol-3-amine (32 mg, 0.24 mmol), Pd₂(dba)₃ (17 mg, 0.002 mmol), Xantphos (21 mg, 0.004 mmol) and sodium tert-butoxide (36 mg, 0.38 mmol) were added in toluene (5 mL) and stirred at 100° C. for 2 hours under nitrogen protection. The reaction solution was concentrated and purified by column chromatography on silica gel (methanol/water) and preparative thin-layer chromatography (dichloromethane/methanol) to give the target product (17 mg, yield 14%) as a yellow solid. [M+H]⁺ 629.2. ¹H NMR (400 MHz, CD₃OD) δ 8.26-8.17 (m, 1H), 7.99-7.90 (m, 1H), 7.82-7.72 (m, 2H), 7.52-7.40 (m, 1H), 7.29-7.17 (m, 1H), 6.82-6.67 (m, 2H), 5.45-5.16 (m, 1H), 4.66-4.56 (m, 1H), 4.36-4.22 (m, 3H), 4.08-3.96 (m, 1H), 3.25-3.15 (m, 2H), 3.04-2.95 (m, 1H), 2.40-2.11 (m, 3H), 2.0-1.88 (m, 3H), 1.66-1.58 (m, 3H), 0.72-0.63 (m, 3H).

The compounds in the table below were prepared from corresponding intermediates and reagents by following the preparation steps of the compound 336:

| | | LC-MS | |
|---|---|---|---|
| Compound | Structural formula | [M + H]⁺ | ¹HNMR |
| 331 | 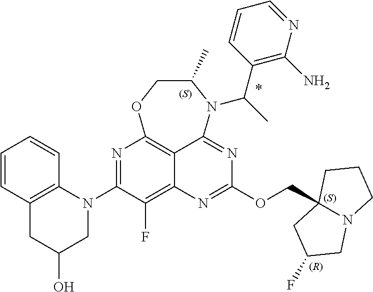 | 645.4 | (400 MHz, CD₃OD) δ 8.02-7.88 (m, 1H), 7.83-7.71 (m, 1H), 7.14-7.06 (m, 1H), 7.05-6.97 (m, 1H), 6.94-6.85 (m, 1H), 6.83-6.66 (m, 3H), 5.39-5.18 (m, 1H), 4.64-4.51 (m, 1H), 4.33-4.18 (m, 4H), 4.09-3.98 (m, 1H), 3.88-3.76 (m, 1H), 3.58-3.48 (m, 1H), 3.26-3.10 (m, 4H), 3.03-2.94 (m, 1H), 2.85-2.72 (m, 1H), 2.37-2.12 (m, 3H), 2.00-1.83 (m, 3H), 1.69-1.54 (m, 3H), 0.71-0.62 (m, 3H). |

### Compound 394 (*)₂-(((1-((((9S)-10-(1-(2-aminopyridin-3-yl)ethyl)-4-fluoro-5-(7-fluoro-6-methyl-5-(trifluoromethyl)-1H-indazol-4-yl)-9-methyl-9,10-dihydro-8H-7-oxa-1,3,6,10-tetraazacyclohepta[de]naphthalen-2-yl)oxy)methyl)cyclopropyl)methyl)(methyl)amino)ethan-1-ol

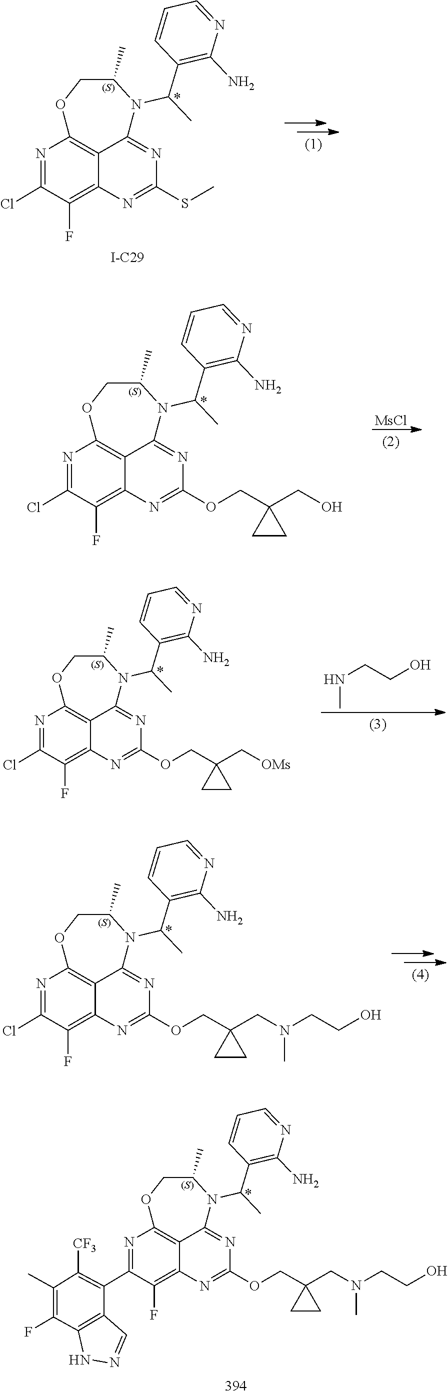

### Step 1: (*)(1-((((9S)-10-(1-(2-aminopyridin-3-yl)ethyl)-5-chloro-4-fluoro-9-methyl-9,10-dihydro-8H-7-oxa-1,3,6,10-tetraazacyclohepta[de]naphthalen-2-yl)oxy)methyl)cyclopropyl)methanol

The target intermediate was prepared from corresponding intermediates and reagents by following the preparation steps 1 and 2 of the compound 1. [M+H]⁺ 475.2

### Step 2: (*)(1-((((9S)-10-(1-(2-aminopyridin-3-yl)ethyl)-5-chloro-4-fluoro-9-methyl-9,10-dihydro-8H-7-oxa-1,3,6,10-tetraazacyclohepta[de]naphthalen-2-yl)oxy)methyl)cyclopropyl)methyl methanesulfonate

At 0° C., DIEA (261 mg, 2.02 mmol) and methanesulfonyl chloride (154 mg, 1.35 mmol) were added to a solution of (*)(1-((((9S)-10-(1-(2-aminopyridin-3-yl)ethyl)-5-chloro-4-fluoro-9-methyl-9,10-dihydro-8H-7-oxa-1,3,6,10-tetraazacyclohepta[de]naphthalen-2-yl)oxy)methyl)cyclopropyl)methanol (320 mg, 0.67 mmol) in dichloromethane (10 mL) and stirred for 1.5 hours. The reaction solution was diluted with dichloromethane and washed with a saturated aqueous solution of sodium chloride. The organic phase was collected and dried with anhydrous sodium sulfate. After filtration, the filtrate was concentrated under reduced pressure to give the target product (440 mg, yield 118%) as a yellow solid, which was used in the reaction of the next step directly. [M+H]⁺ 552.6

### Step 3: (*)₂-(((1-((((9S)-10-(1-(2-aminopyridin-3-yl)ethyl)-5-chloro-4-fluoro-9-methyl-9,10-dihydro-8H-7-oxa-1,3,6,10-tetraazacyclohepta[de]naphthalen-2-yl)oxy)methyl)cyclopropyl)methyl)(methyl)amino)ethan-1-ol

(*)(1-((((9S)-10-(1-(2-aminopyridin-3-yl)ethyl)-5-chloro-4-fluoro-9-methyl-9,10-dihydro-8H-7-oxa-1,3,6,10-tetraazacyclohepta[de]naphthalen-2-yl)oxy)methyl)cyclopropyl)methyl methanesulfonate (220 mg, 0.40 mmol), 2-(methylamino)ethan-1-ol (90 mg, 1.19 mmol) and DIEA (206 mg, 1.59 mmol) were added in DMF (3 mL) and stirred at 90° C. for 4 hours under nitrogen protection. The reaction solution was concentrated under reduced pressure and purified by column chromatography on silica gel (methanol/water) to give the target product (150 mg, yield 71%) as a yellow solid. [M+H]⁺ 531.8

### Step 4: (*)₂-(((1-((((9S)-10-(1-(2-aminopyridin-3-yl)ethyl)-4-fluoro-5-(7-fluoro-6-methyl-5-(trifluoromethyl)-1H-indazol-4-yl)-9-methyl-9,10-dihydro-8H-7-oxa-1,3,6,10-tetraazacyclohepta[de]naphthalen-2-yl)oxy)methyl)cyclopropyl)methyl)(methyl)amino)ethan-1-ol

The target compound was prepared from corresponding intermediates and reagents by following the preparation steps 3 and 4 of the compound 1. [M+H]⁺ 713.7. ¹H NMR (400 MHz, CD₃OD) δ 8.02-7.94 (m, 1H), 7.88-7.69 (m, 2H), 6.83-6.72 (m, 2H), 4.68-4.55 (m, 1H), 4.55-4.29 (m, 3H), 4.14-3.98 (m, 1H), 3.65 (t, J=6.0 Hz, 2H), 2.69-2.46 (m, 7H), 2.35 (s, 3H), 1.68 (d, J=6.8 Hz, 3H), 0.81-0.62 (m, 5H), 0.61-0.43 (m, 2H).

### Compounds 169 and 170 (*)3-(1-(5-(5-amino-4-fluoro-3-methyl-2-(trifluoromethyl)phenyl)-4-fluoro-2-(((2S or 2R,7aS)-2-fluorotetrahydro-1H-pyrrolizin-7a(5H)-yl-2-d)methoxy)-8,9-dihydro-1OH-7-oxa-1,3,6,10-tetraazacyclohepta[de]naphthalen-10-yl)ethyl)pyridin-2-amine (diastereomer 1 and diastereomer 2)

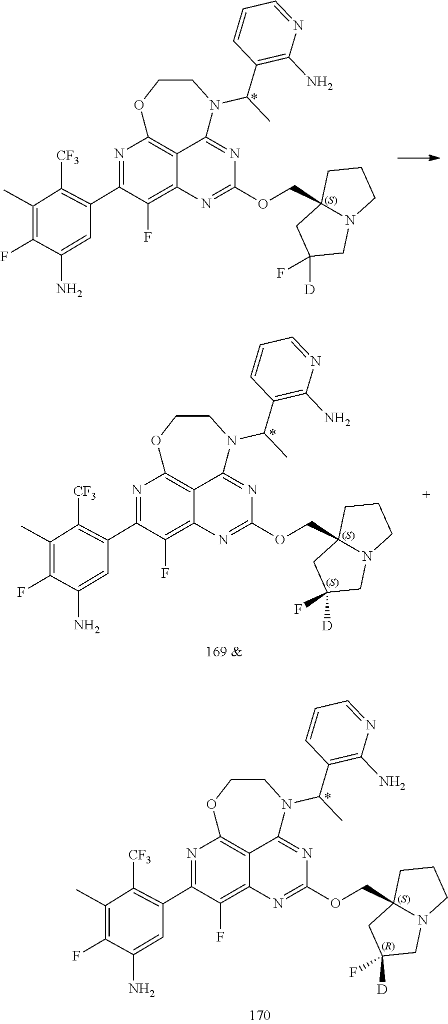

The compound prepared from I-A18, I-B31 and I-C9 by following the preparation steps of the compound 2 was resolved by means of preparative high performance HPLC to obtain one pair of diastereomers. Preparative HPLC conditions: column: AQ-C18 (3×15 cm); mobile phase: methanol/water (0.1% formic acid); flow rate: 30 mL/minute; detector: UV 254 nm.

First eluate (compound 169, diastereomer 1, chiral HPLC analysis conditions: column: ODH (0.46×25 cm); mobile phase: n-heptane/ethanol (0.1% diethylamine)=20/80, RT=5.848 minutes), de %=100%, [M+H]⁺ 676.3. ¹H NMR (400 MHz, CD₃OD) δ 7.96-7.94 (m, 1H), 7.75 (d, J=7.4 Hz, 1H), 6.79-6.75 (m, 1H), 6.69-6.48 (m, 2H), 4.52-4.19 (m, 4H), 3.74-3.68 (m, 1H), 3.52-3.32 (m, 2H), 3.10-3.05 (m, 1H), 2.96-2.81 (m, 1H), 2.78-2.68 (m, 1H), 2.55-2.42 (m, 1H), 2.35 (s, 3H), 2.27-2.09 (m, 1H), 2.05-1.77 (m, 4H), 1.63 (d, J=6.8 Hz, 3H).

Second eluate (compound 170, diastereomer 2, chiral HPLC analysis conditions: column: ODH (0.46×25 cm); mobile phase: n-heptane/ethanol (0.10% diethylamine)=20/80, RT=6.277 minutes), de %=100%, [M+H]⁺ 676.3. ¹H NMR (400 MHz, CD₃OD) δ 7.95-7.93 (m, 1H), 7.74 (d, J=7.5 Hz, 1H), 6.78-6.74 (m, 1H), 6.64-6.48 (m, 2H), 4.50-4.24 (m, 4H), 3.78-3.66 (m, 1H), 3.58-3.48 (m, 1H), 3.33-3.16 (m, 3H), 3.07-2.98 (m, 1H), 2.41-2.12 (m, 6H), 2.05-1.85 (m, 3H), 1.63 (d, J=6.9 Hz, 3H).

The compounds in the table below were prepared by following the preparative HPLC conditions of compounds 169 and 170:

| Compound | Structural formula | Name |
|---|---|---|
| 178 | 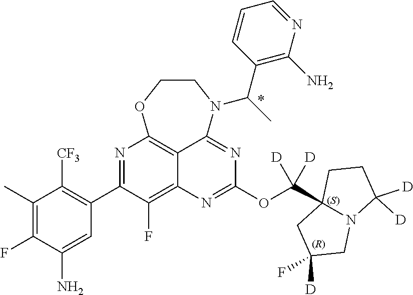 | (*)3-(1-(5-(5-amino-4-fluoro-3- methyl-2-(trifluoromethyl)phenyl)- 4-fluoro-2-(((2R or 2S,7aS)-2- fluorotetrahydro-1H-pyrrolizin- 7a(5H)-yl-2,5,5-d₃)methoxy-d₂)- 8,9-dihydro-10H-7-oxa-1,3,6,10- tetraazacyclohepta[de]naphthalen- 10-yl)ethyl)pyridin-2-amine (diastereomer 1) |
| | + | |
| 179 | 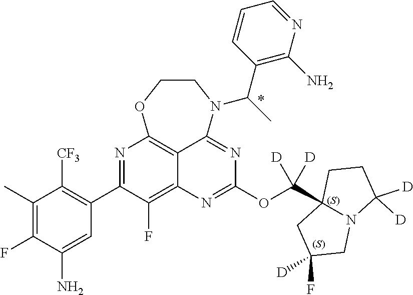 | (*)3-(1-(5-(5-amino-4-fluoro-3- methyl-2-(trifluoromethyl)phenyl)- 4-fluoro-2-(((2R or 2S,7aS)-2- fluorotetrahydro-1H-pyrrolizin- 7a(5H)-yl-2,5,5-d3)methoxy-d2)- 8,9-dihydro-10H-7-oxa-1,3,6,10- tetraazacyclohepta[de]naphthalen- 10-yl)ethyl)pyridin-2-amine (diastereomer 2) |
| | 178 & 179 | |
| 238 | 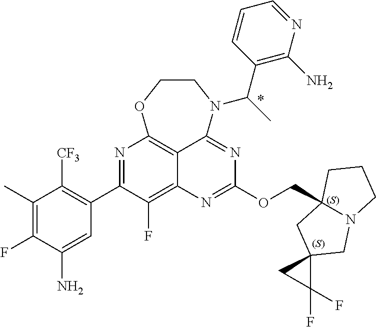 | (*)3-(1-(5-(5-amino-4-fluoro-3- methyl-2-(trifluoromethyl)phenyl)- 2-(((1R or 1S,7a′S)-2,2- difluorodihydro-1′H,3′H- spiro[cyclopropane-1,2′-pyrrolizin]- 7a′(5′H)-yl)methoxy)-4-fluoro-8,9- dihydro-10H-7-oxa-1,3,6,10- tetraaza cyclohepta[de]naphthalen- 10-yl)ethyl)pyridin-2-amine (diastereomer 1) |
| | + | |
| 239 | 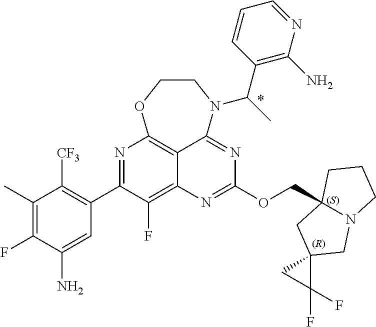 | (*)3-(1-(5-(5-amino-4-fluoro-3- methyl-2-(trifluoromethyl)phenyl)- 2-(((1R or 1S,7a′S)-2,2- difluorodihydro-1′H,3′H- spiro[cyclopropane-1,2′-pyrrolizin]- 7a′(5′H)-yl)methoxy)-4-fluoro-8,9- dihydro-10H-7-oxa-1,3,6,10- tetraaza cyclohepta[de]naphthalen- 10-yl)ethyl)pyridin-2-amine (diastereomer 2) |
| | 238 & 239 | |
| 241 | 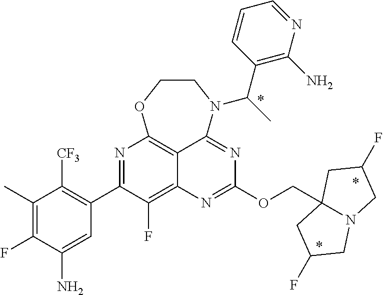 [SMILES: Cc1c(F)c(N)cc(-c2nc3c4c(nc(OCC56CC(F)CN5CC(F)C6)nc4c2F)N(C(C)c2cccnc2N)CCO3)c1C(F)(F)F] | (*)3-(1-(5-(5-amino-4-fluoro-3- methyl-2-(trifluoromethyl)phenyl)- 2-(((2R or 2S,6R or 6S)-2,6- difluorotetrahydro-1H-pyrrolizin- 7a(5H)-yl)methoxy)-4-fluoro-8,9- dihydro-10H-7-oxa-1,3,6,10- tetraazacyclohepta[de]naphthalen- 10-yl)ethyl)pyridin-2-amine (diastereomer 1) |
| | + | |
| 242 | 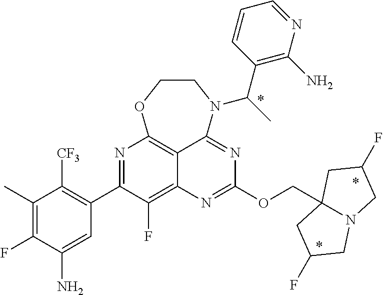 [SMILES: Cc1c(F)c(N)cc(-c2nc3c4c(nc(OCC56CC(F)CN5CC(F)C6)nc4c2F)N(C(C)c2cccnc2N)CCO3)c1C(F)(F)F] | (*)3-(1-(5-(5-amino-4-fluoro-3- methyl-2-(trifluoromethyl)phenyl)- 2-(((2R or 2S,6R or 6S)-2,6- difluorotetrahydro-1H-pyrrolizin- 7a(5H)-yl)methoxy)-4-fluoro-8,9- dihydro-10H-7-oxa-1,3,6,10- tetraazacyclohepta[de]naphthalen- 10-yl)ethyl)pyridin-2-amine (diastereomer 2) |
| | 241 & 242 | |
| 245 | 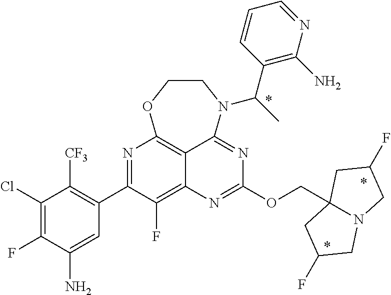 [SMILES: CC(c1cccnc1N)N1CCOc2nc(-c3cc(N)c(F)c(Cl)c3C(F)(F)F)c(F)c3nc(OCC45CC(F)CN4CC(F)C5)nc1c23] | (*)3-(1-(5-(5-amino-3-chloro-4- fluoro-2-(trifluoromethyl)phenyl)- 2-(((2R or 2S,6R or 6S)-2,6- difluorotetrahydro-1H-pyrrolizin- 7a(5H)-yl)methoxy)-4-fluoro-8,9- dihydro-10H-7-oxa-1,3,6,10- tetraazacyclohepta[de]naphthalen- 10-yl)ethyl)pyridin-2-amine (diastereomer 1) |
| | + | |
| 246 | 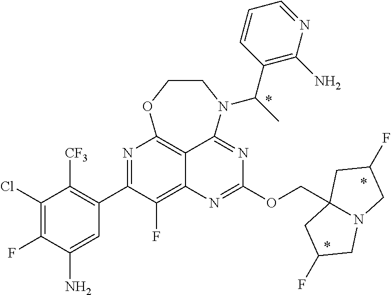 [SMILES: CC(c1cccnc1N)N1CCOc2nc(-c3cc(N)c(F)c(Cl)c3C(F)(F)F)c(F)c3nc(OCC45CC(F)CN4CC(F)C5)nc1c23] | (*)3-(1-(5-(5-amino-3-chloro-4- fluoro-2-(trifluoromethyl)phenyl)- 2-(((2R or 2S,6R or 6S)-2,6- difluorotetrahydro-1H-pyrrolizin- 7a(5H)-yl)methoxy)-4-fluoro-8,9- dihydro-10H-7-oxa-1,3,6,10- tetraazacyclohepta[de]naphthalen- 10-yl)ethyl)pyridin-2-amine (diastereomer 2) |
| | 245 & 246 | |
| 338 | 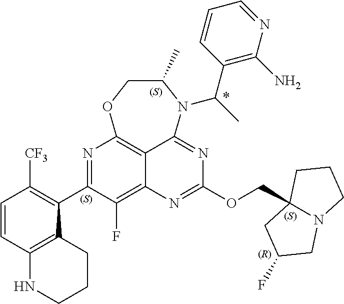 | (*)3-(1-((5R or 5S,9S)-4-fluoro-2- (((2R,7aS)-2-fluorotetrahydro-1H- pyrrolizin-7a(5H)-yl)methoxy)-9- methyl-5-(6-(trifluoromethyl)- 1,2,3,4-tetrahydroquinolin-5-yl)- 8,9-dihydro-10H-7-oxa-1,3,6,10- tetraazacyclohepta[de]naphthalen- 10-yl)ethyl)pyridin-2-amine (diastereomer 1) |
| | + | |
| 339 | 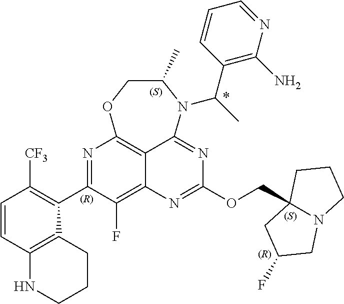 | (*)3-(1-((5R or 5S,9S)-4-fluoro-2- (((2R,7aS)-2-fluorotetrahydro-1H- pyrrolizin-7a(5H)-yl)methoxy)-9- methyl-5-(6-(trifluoromethyl)- 1,2,3,4-tetrahydroquinolin-5-yl)- 8,9-dihydro-10H-7-oxa-1,3,6,10- tetraazacyclohepta[de]naphthalen- 10-yl)ethyl)pyridin-2-amine (diastereomer 2) |
| | 338 δ 339 | |

| | LC-MS | | |
|---|---|---|---|
| Compound | [M + H]⁺ | ¹HNMR | |
| 178 | 680.3 | (400 MHz, CD₃OD) δ 7.97-7.91 | It was obtained by |
| | | (m, 1H), 7.76-7.70 (m, 1H), | resolving the |
| | | 6.79-6.73 (m, 1H), 6.65-6.52 (m, | compound |
| | | 2H), 4.49-4.38 (m, 1H), 4.36- | prepared from I- |
| | | 4.26 (m, 1H), 3.79-3.67 (m, 1H), | A18, I-B33 and I- |
| | | 3.57-3.47 (m, 1H), 3.29-3.16 (m, | C9 by following |
| | | 2H), 2.39-2.10 (m, 6H), 2.05- | the preparation |
| | | 1.84 (m, 3H), 1.67-1.60 (m, 3H). | steps of the |
| 179 | 680.3 | (400 MHz, CD₃OD) δ 7.98-7.93 | compound 2 |
| | | (m, 1H), 7.78-7.72 (m, 1H), | through |
| | | 6.81-6.75 (m, 1H), 6.65-6.54 (m, | preparative HPLC; |
| | | 2H), 4.50-4.25 (m, 2H), 3.82- | preparative HPLC |
| | | 3.64 (m, 1H), 3.64-3.41 (m, 2H), | conditions: |
| | | 3.16-2.95 (m, 1H), 2.60-2.14 (m, | column: AQ-C18 |
| | | 5H), 2.09-1.87 (m, 4H), 1.68- | (3 × 15 cm); |
| | | 1.61 (m, 3H). | mobile phase: |
| | | | methanol/water |
| | | | (0.1% formic acid). |
| 238 | 719.3 | (400 MHz, CD₃OD) δ 7.98-7.90 | It was obtained by |
| | | (m, 1H), 7.77-7.72 (m, 1H), | resolving the |
| | | 6.80-6.74 (m, 1H), 6.65-6.55 (m, | compound 228; |
| | | 2H), 4.48-4.26 (m, 4H), 3.78- | chiral HPLC |
| | | 3.69 (m, 1H), 3.56-3.48 (m, 1H), | resolution |
| | | 3.29-3.24 (m, 1H), 3.19-3.12 (m, | conditions: |
| | | 1H), 2.85-2.80 (m, 1H), 2.73- | column: IG (2 × 25 |
| | | 2.65 (m, 1H), 2.39-2.34 (m, 3H), | cm); mobile phase: |
| | | 2.31-2.24 (m, 1H), 2.19-2.12 (m, | n-heptane/ethanol |
| | | 1H), 2.04-1.98 (m, 1H), 1.95- | (0.1% ammonia |
| | | 1.74 (m, 3H), 1.67-1.61 (m, 3H), | water) = 70:30. |
| | | 1.50-1.37 (m, 2H). | |
| 239 | 719.3 | (400 MHz, CD₃OD) δ 7.98-7.90 | |
| | | (m, 1H), 7.78-7.70 (m, 1H), | |
| | | 6.80-6.73 (m, 1H), 6.69-6.55 (m, | |
| | | 2H), 4.47-4.23 (m, 4H), 3.76- | |
| | | 3.65 (m, 1H), 3.54-3.44 (m, 1H), | |
| | | 3.25-3.18 (m, 1H), 3.12-3.04 (m, | |
| | | 1H), 2.91-2.77 (m, 2H), 2.40- | |
| | | 2.34 (m, 3H), 2.33-2.26 (m, 1H), | |
| | | 2.17-2.08 (m, 1H), 2.08-1.88 (m, | |
| | | 4H), 1.68-1.59 (m, 3H), 1.47- | |
| | | 1.40 (m, 2H). | |
| 241 | 693.3 | (400 MHz, CD₃OD) δ 7.97-7.91 | It was obtained by |
| | | (m, 1H), 7.77-7.71 (m, 1H), | resolving the |
| | | 6.80-6.73 (m, 1H), 6.65-6.53 (m, | compound |
| | | 2H), 5.43-5.21 (m, 2H), 4.57- | prepared from I- |
| | | 4.23 (m, 4H), 3.79-3.65 (m, 1H), | |
| 242 | 693.2 | 3.58-3.45 (m, 2H), 3.28-3.09 (m, 3H), 2.53-2.15 (m, 7H), 1.67-1.57 (m, 3H). (400 MHz, CD₃OD) δ 7.98-7.90 (m, 1H), 7.78-7.69 (m, 1H), 6.81-6.72 (m, 1H), 6.67-6.51 (m, 2H), 5.43-5.20 (m, 2H), 4.54- 4.25 (m, 4H), 3.75-3.64 (m, 1H), 3.55-3.43 (m, 2H), 3.29-3.10 (m, 3H), 2.51-2.17 (m, 7H), 1.67-1.58 (m, 3H). | 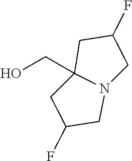 [SMILES: OCC12CC(F)CN1CC(F)C2] |
| | | A18, I-B38 and | |
| | | I-C9 by following | |
| | | the preparation | |
| | | steps of the compound 2; | |
| | | chiral HPLC resolution | |
| | | conditions: | |
| | | column: ODH (2 × | |
| | | 25 cm); mobile phase: n- | |
| | | heptane/ethanol | |
| | | (0.1% ammonia water) = | |
| | | 50:50. | |
| 245 | 713.2 | (400 MHz, CD₃OD) δ 7.96-7.92 | It was obtained by |
| | | (m, 1H), 7.74 (d, J = 7.4 Hz, | separating the compound |
| | | 1H), 6.83-6.73 (m, 1H), 6.67 (d, | prepared from I- |
| | | J = 8.1 Hz, 1H), 6.64-6.52 (m, | |
| 246 | 713.2 | 1H), 5.45-5.22 (m, 2H), 4.53-4.27 (m, 4H), 3.78-3.65 (m, 1H), 3.60-3.43 (m, 2H), 3.29- 3.15 (m, 3H), 2.54-2.15 (m, 4H), 1.63 (d, J = 6.9 Hz, 3H). (400 MHz, CD₃OD) δ 8.00-7.87 (m, 1H), 7.73 (d, J = 7.5 Hz, 1H), 6.83-6.72 (m, 1H), 6.67 (d, J = 8.1 Hz, 1H), 6.64-6.52 (m, 1H), 5.44-5.17 (m, 2H), 4.54-4.25 (m, 4H), 3.78-3.62 (m, 1H), 3.57-3.41 (m, 2H), 3.29- 3.07 (m, 3H), 2.53-2.11 (m, 4H), 1.63 (d, J = 6.9 Hz, 3H). | 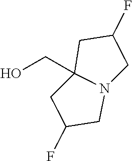 [SMILES: OCC12CC(F)CN1CC(F)C2] A28, I-B38 and I-C9 by |
| | | | following the preparation steps |
| | | | of the compound 2 through |
| | | | preparative thin-layer |
| | | | chromatography (dichloromethane/ |
| | | | methanol = 10/1): compound 245 |
| | | | (R_{f} = 0.6), compound 246 |
| | | | (R_{f} = 0.5). |
| 338 | 697.2 | (400 MHz, CD₃OD) δ 8.02-7.92 | It was obtained by |
| | | (m, 1H), 7.83-7.76 (m, 1H), | resolving the |
| | | 7.29-7.23 (m, 1H), 6.83-6.71 (m, | compound |
| | | 2H), 6.64-6.56 (m, 1H), 5.42- | prepared from I- |
| | | 5.21 (m, 1H), 4.65-4.56 (m, 1H), | A68, I-B1 and I- |
| | | 4.36-4.27 (m, 3H), 4.12-4.01 (m, | C29 by following |
| | | 1H), 3.28-3.16 (m, 5H), 3.07- | the preparation |
| | | 2.96 (m, 1H), 2.5-2.11 (m, 5H), | steps of the |
| | | 2.03-1.87 (m, 3H), 1.85-1.78 (m, | compound 1; |
| | | 2H), 1.72-1.64 (m, 3H), 0.73- | chiral HPLC |
| | | 0.64 (m, 3H). | resolution |
| 339 | 697.2 | (400 MHz, CD₃OD) δ 7.99-7.93 | conditions: |

| | |
|---|---|
| (m, 1H), 7.82-7.76 (m, 1H), | column: ODH (2 × |
| 7.34-7.25 (m, 1H), 6.82-6.71 (m, | 25 cm); mobile |
| 2H), 6.65-6.56 (m, 1H), 5.42- | phase: n- |
| 5.20 (m, 1H), 4.65-4.55 (m, 1H), | heptane/ethanol |
| 4.36-4.26 (m, 3H), 4.11-4.02 (m, | (0.1% ammonia |
| 1H), 3.27-3.16 (m, 5H), 3.06- | water) = 70:30. |
| 2.94 (m, 1H), 2.41-2.13 (m, 5H), | |
| 2.04-1.89 (m, 3H), 1.82-1.71 (m, | |
| 2H), 1.71-1.63 (m, 3H), 0.72- | |
| 0.64 (m, 3H). | |

Note:
the intermediate I-B38 was commercially available.

The diastereomers in the table were subjected to chiral HPLC analysis under the following conditions (flow rate: 15 mL/minute; detector: UV 254 nm):

| Compound | Column | Mobile phase | RT/minute | de % |
|---|---|---|---|---|
| 178 | ODH (0.46 × 25 cm) | n-Heptane/ethanol (0.1% diethylamine) = | 6.179 | 100% |
| 179 | | 20/80 | 5.788 | 100% |
| 238 | IG (0.46 × 25 cm) | n-Heptane/ethanol (0.1% diethylamine) = | 14.796 | 100% |
| 239 | | 70/30 | 22.867 | 95.91% |
| 241 | ODH (0.46 × 25 cm) | n-Heptane/ethanol (0.1% diethylamine) = | 7.964 | 100% |
| 242 | | 50/50 | 8.657 | 100% |
| 338 | ODH (0.46 × 25 cm) | n-Heptane/ethanol (0.1% diethylamine) = | 6.110 | 100% |
| 339 | | 70/30 | 9.358 | 100% |

### Compound 293 (*)3-((9S)-10-(1-(2-aminopyridin-3-yl)ethyl)-4-fluoro-2-(((2R,7aS)-2-fluorotetrahydro-1H-pyrrolizin-7a(5H)-yl)methoxy)-9-methyl-9,10-dihydro-8H-7-oxa-1,3,6,10-tetraazacyclohepta[de]naphthalen-5-yl)-5-chloro-4-cyclopropylphenyl ethylcarbamate acetate

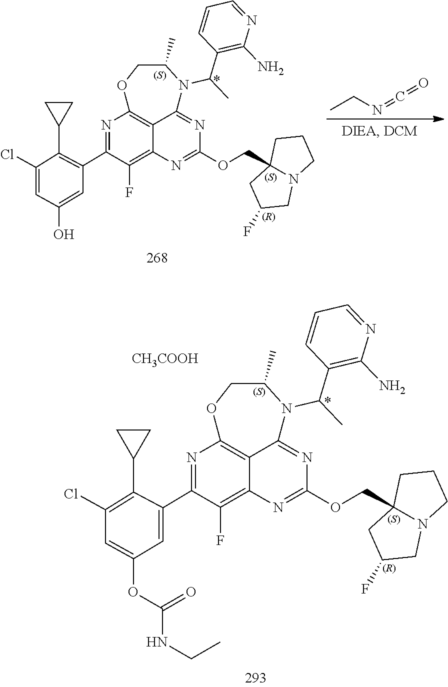

At 0-5° C., isocyanoethane (93 mg, 1.3 mmol) was added to a solution of the compound 268 (312 mg, 0.47 mmol) and DIEA (315 mg, 5.2 mmol) in DCM (20 mL) and stirred at room temperature for 1 hour. A saturated aqueous solution of ammonium 10 chloride (0.2 mL) was added, and the reaction solution was concentrated under reduced pressure and purified by column chromatography on silica gel (water/methanol, 0.2% acetic acid) to give the target product (182 mg, yield 49%) as a white solid. (Free base) [M+H]⁺ 735.3. ¹H NMR (400 MHz, CD₃OD) δ 8.02-7.92 (m, 1H), 7.84-7.76 (m, 1H), 7.34-7.29 (m, 1H), 7.21-7.13 (m, 1H), 6.83-6.66 (m, 2H), 5.57-5.27 (m, 1H), 4.67-4.58 (m, 1H), 4.56-4.45 (m, 2H), 4.34-4.25 (m, 1H), 4.17-4.02 (m, 1H), 3.71-3.42 (m, 3H), 3.25-3.12 (m, 3H), 2.58-2.23 (m, 3H), 2.19-2.01 (m, 3H), 1.97-1.87 (m, 5H), 1.70-1.61 (m, 3H), 1.17-1.11 (m, 3H), 0.79-0.64 (m, 5H), 0.26-0.30 (m, 2H).

The compounds in the table below were prepared from corresponding intermediates and reagents by following the preparation steps of the compound 293:

| | | LC-MS | |
|---|---|---|---|
| Compound | Structural formula | [M + H]⁺ | ¹HNMR |
| 294 | 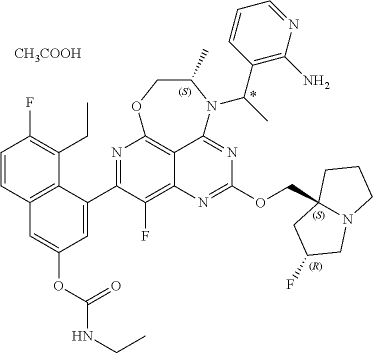 | 757.4 (Free base) | (400 MHz, CD₃OD) δ 7.98 (s, 1H), 7.88-7.72 (m, 3H), 7.49-7.21 (m, 2H), 6.82-6.65 (m, 2H), 5.54-5.27 (m, 1H), 4.68-4.58 (m, 1H), 4.56- 4.46 (m, 2H), 4.38-4.23 (m, 1H), 4.15-4.00 (m, 1H), 3.69-3.37 (m, 3H), 3.24-3.11 (m, 3H), 2.64-2.23 (m, 5H), 2.15-1.99 (m, 3H), 1.91 (s, 4H), 1.72-1.59(m, 3H), 1.26-1.12 (m, 3H), 0.95-0.87 (m, 1H), 0.86-0.78 (m, 2H), 0.74-0.65 (m, 3H). |
| 295 | 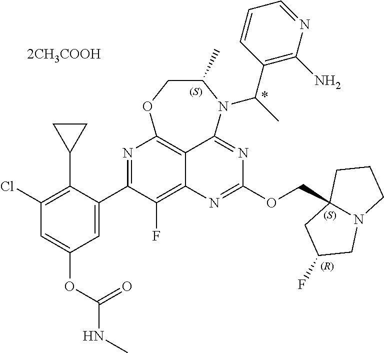 | 721.3 (Free base) | (400 MHz, CD₃OD) δ 8.09-7.69 (m, 2H), 7.45-7.08 (m, 2H), 6.89-6.60 (m, 2H), 5.55-5.36 (m, 1H), 4.70- 4.48 (m, 3H), 4.38-4.23 (m, 1H), 4.16-3.99 (m, 1H), 3.79-3.51 (m, 3H), 3.39-3.16 (m, 2H), 2.87-2.36 (m, 5H), 2.35-2.04 (m, 4H), 1.91 (s, 6H), 1.76-1.55 (m, 3H), 0.89-0.55 (m, 5H), 0.28-0.00 (m, 2H). |
| 296 | 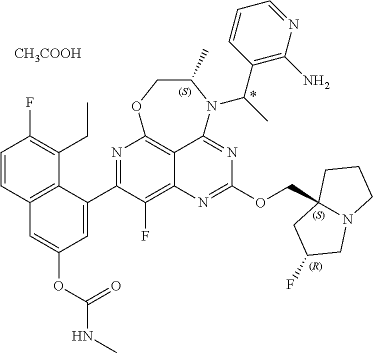 | 743.4 (Free base) | (400 MHz, CD₃OD) δ 8.02-7.92 (m, 1H), 7.88-7.72 (m, 3H), 7.40-7.18 (m, 2H), 6.82-6.71 (m, 2H), 5.43- 5.24 (m, 1H), 4.66-4.54 (m, 1H), 4.47-4.25 (m, 3H), 4.14-4.02 (m, 1H), 3.47-3.30 (m, 3H), 3.14-3.03 (m, 1H), 2.82-2.74 (m, 3H), 2.69- 2.14 (m, 5H), 2.09-1.94 (m, 3H), 1.93-1.88 (m, 2H), 1.72-1.65 (m, 3H), 0.96-0.62 (m, 6H). |
| 301 | 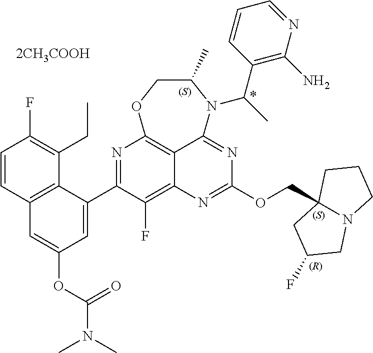 | 757.4 (Free base) | (400 MHz, CD₃OD) δ 8.02-7.95 (m, 1H), 7.88-7.75 (m, 3H), 7.43-7.20 (m, 2H), 6.82-6.72 (m, 2H), 5.48- 5.30 (m, 1H), 4.66-4.60 (m, 1H), 4.52-4.44 (m, 2H), 4.38-4.29 (m, 1H), 4.15-4.05 (m, 1H), 3.60-3.43 (m, 3H), 3.22-3.11 (m, 4H), 3.04- 2.94 (m, 3H), 2.68-2.23 (m, 5H), 2.17-2.00 (m, 3H), 1.99-1.91 (m, 6H), 1.75-1.64 (m, 3H), 0.93-0.82 (m, 3H), 0.76-0.67 (m, 3H). |
| 304 | 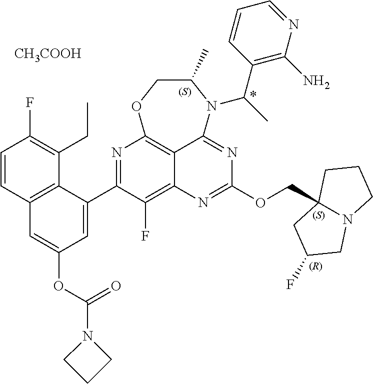 | 769.2 (Free base) | (400 MHz, CD₃OD) δ 8.01-7.94 (m, 1H), 7.89-7.75 (m, 3H), 7.43-7.22 (m, 2H), 6.84-6.70 (m, 2H), 5.48- 5.25 (m, 1H), 4.64-4.59 (m, 1H), 4.44-4.32 (m, 3H), 4.31-4.23 (m, 2H), 4.12-4.03 (m, 3H), 3.45-3.34 (m, 3H), 3.14-3.08 (m, 1H), 2.67- 2.27 (m, 7H), 2.11-1.97 (m, 3H), 1.95-1.88 (m, 3H), 1.71-1.65 (m, 3H), 0.91-0.80 (m, 3H), 0.73-0.67 (m, 3H). |
| 307 | 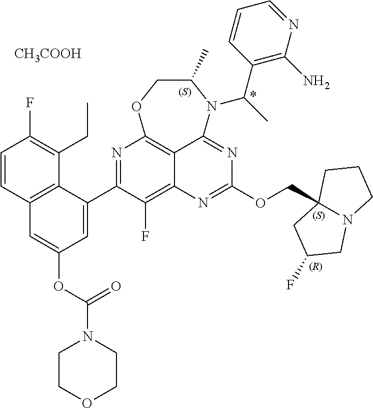 | 799.4 (Free base) | (400 MHz, CD₃OD) δ 8.01-7.93 (m, 1H), 7.88-7.84 (m, 1H), 7.81-7.74 (m, 2H), 7.47-7.26 (m, 2H), 6.82- 6.69 (m, 2H), 5.48-5.22 (m, 1H), 4.67-4.58 (m, 1H), 4.47-4.26 (m, 3H), 4.14-4.02 (m, 1H), 3.80-3.65 (m, 6H), 3.57-3.45 (m, 2H), 3.42- 3.31 (m, 2H), 3.28-3.22 (m, 1H), 3.14-3.01 (m, 1H), 2.65-2.15 (m, 5H), 2.10-1.81 (m, 5H), 1.74-1.59 (m, 3H), 0.93-0.881 (m, 1H), 0.85- 0.81 (m, 2H), 0.73-0.66 (m, 3H). |

### Compound 310 (*)5,6-Difluoro-4-((9S)-4-fluoro-2-(((2R,7aS)-2-fluorotetrahydro-1H-pyrrolizin-7a(5H)-yl)methoxy)-10-(1-(imidazo[1,2-a]pyridin-8-yl)ethyl)-9-methyl-9,10-dihydro-8H-7-oxa-1,3,6,10-tetraazacyclohepta[de]naphthalen-5-yl)naphthalen-2-ol

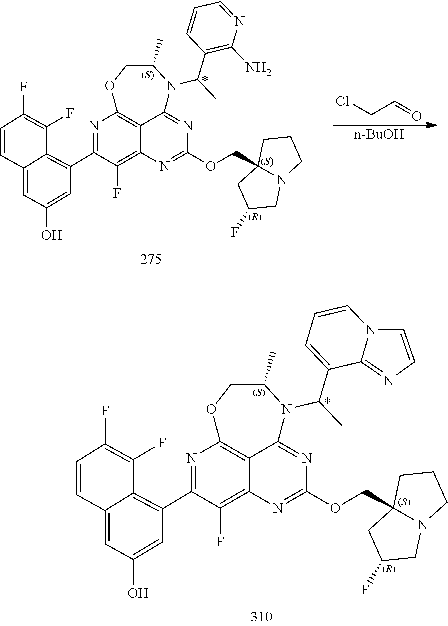

The compound 275 (100 mg, 0.15 mmol) and a 40% aqueous chloroacetaldehyde solution (0.3 mL) were dissolved in n-butyl alcohol (2 mL) and stirred at 100° C. for 2 hours. The reaction solution was concentrated under reduced pressure and purified by column chromatography on silica gel (water/methanol) and preparative thin-layer 10 chromatography (dichloromethane/methanol) to give the target product (45 mg, yield 43%) as a white solid. [M+H]⁺ 700.3. ¹H NMR (400 MHz, CD₃OD) δ 8.49 (d, J=6.8 Hz, 1H), 7.88 (s, 1H), 7.60-7.47 (m, 3H), 7.41-7.30 (m, 1H), 7.29-7.08 (m, 2H), 7.05-6.89 (m, 2H), 5.40-5.21 (m, 1H), 4.63-4.56 (m, 1H), 4.47-4.33 (m, 2H), 4.33-4.15 (m, 2H), 3.38-3.30 (m, 1H), 3.25-3.14 (m, 2H), 3.05-2.95 (m, 1H), 2.46-2.06 (m, 3H), 2.03-1.89 (m, 2H), 1.89-1.74 (m, 4H), 0.62-0.45 (m, 3H).

### Compound 334 (*)5-Chloro-3-(1-((9S)-4-fluoro-2-(((2R,7aS)-2-fluorotetrahydro-1H-pyrrolizin-7a(5H)-yl)methoxy)-9-methyl-5-(6-methyl-5-(trifluoromethyl)-1H-indazol-4-yl)-8,9-dihydro-1OH-7-oxa-1,3,6,10-tetraazacyclohepta[de]naphthalen-10-yl)ethyl)pyridin-2-amine

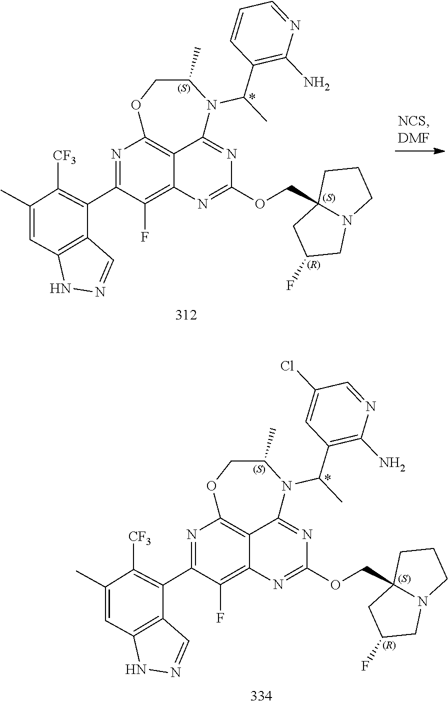

The compound 312 (32 mg, 0.046 mmol) and NCS (9.2 mg, 0.069 mmol) were dissolved in DMF (1 mL) and stirred at room temperature for 4 hours. The reaction solution was purified by column chromatography on silica gel (water/methanol) and 5 preparative thin-layer chromatography (dichloromethane/methanol) to give the target product (8 mg, yield 24%) as a yellow solid. [M+H]⁺ 730.1. ¹H NMR (400 MHz, CD₃OD) δ 7.99-7.95 (m, 1H), 7.82-7.78 (m, 1H), 7.74-7.59 (m, 2H), 6.79-6.73 (m, 1H), 5.38-5.21 (m, 1H), 4.66-4.58 (m, 1H), 4.38-4.30 (m, 3H), 4.15-4.08 (m, 1H), 3.25-3.15 (m, 3H), 3.05-2.97 (m, 1H), 2.69-2.66 (m, 3H), 2.37-2.15 (m, 3H), 2.03-1.91 (m, 3H), 1.69-1.66 (m, 3H), 0.80-0.73 (m, 3H).

The compounds in the table below were prepared from corresponding intermediates and reagents by following the preparation steps of the above compounds under conditions considered appropriate by those skilled in the art:

| | | Molecular |
|---|---|---|
| Compound | Structural formula | weight |
| 234 | 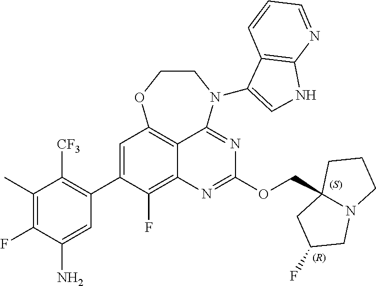 | 669.63 |
| 235 | 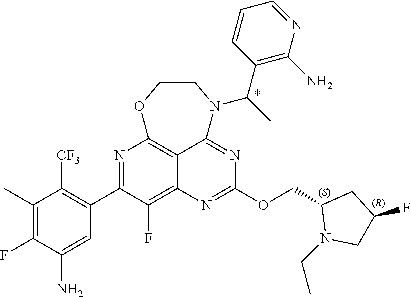 | 662.64 |
| 236 | 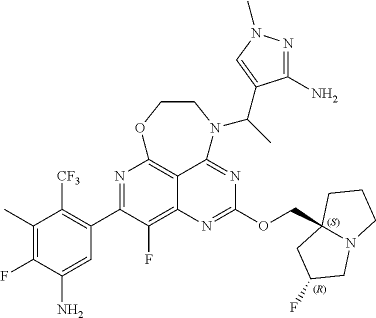 | 677.66 |
| 404 | 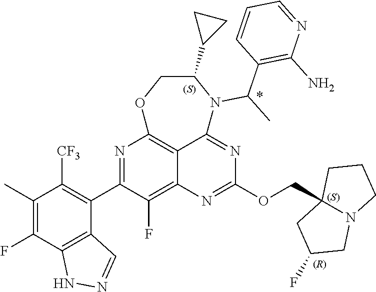 | 739.73 |
| 405 | 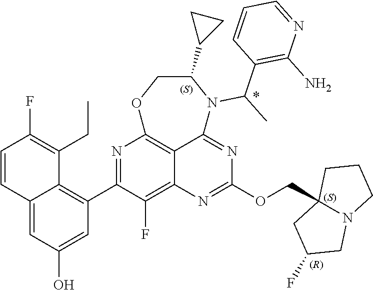 | 711.79 |
| 406 | 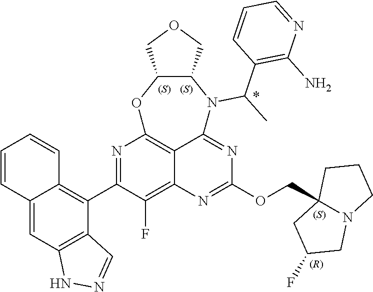 | 691.74 |
| 344 | 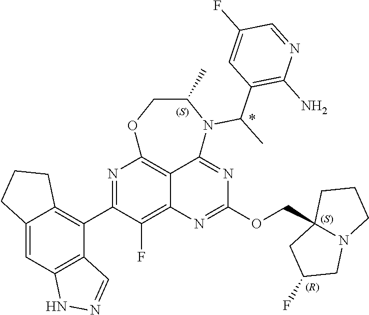 | 671.73 |
| 346 | 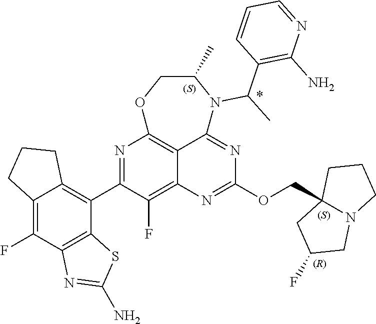 | 703.79 |
| 347 | 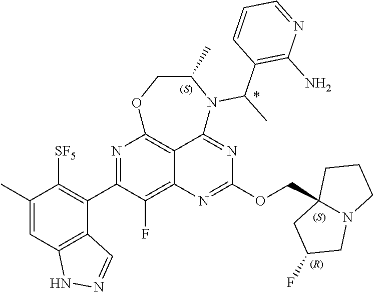 | 753.74 |
| 349 | 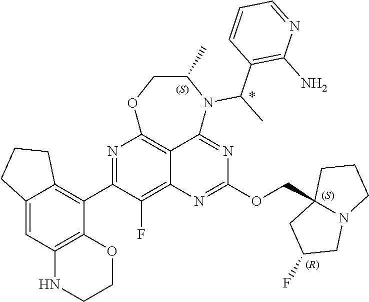 | 670.77 |
| 350 | 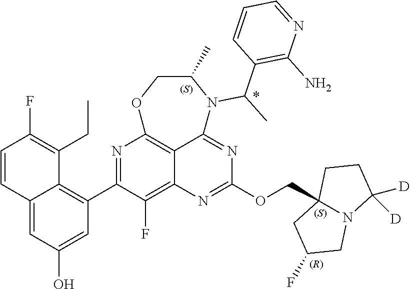 | 687.76 |
| 351 | 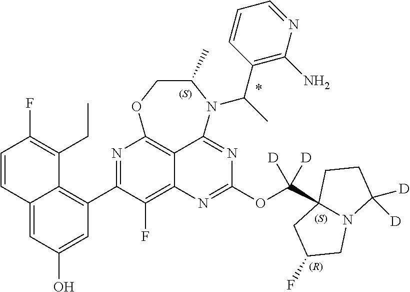 | 689.78 |
| 352 | 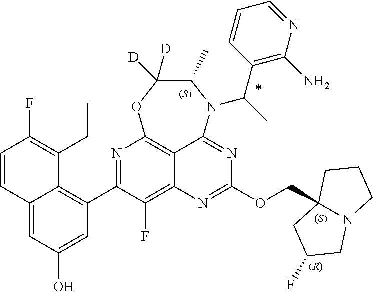 | 687.76 |
| 407 | 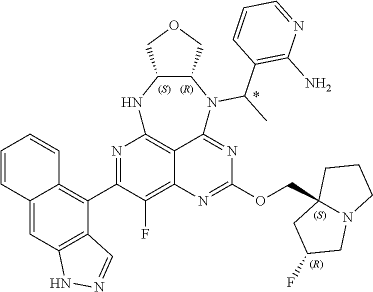 | 690.76 |
| 408 | 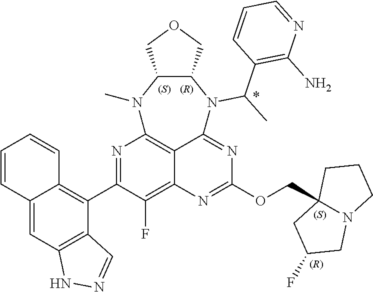 | 704.79 |
| 409 | 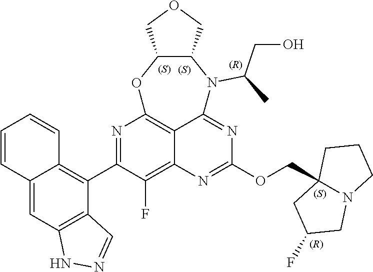 | 629.67 |
| 410 | 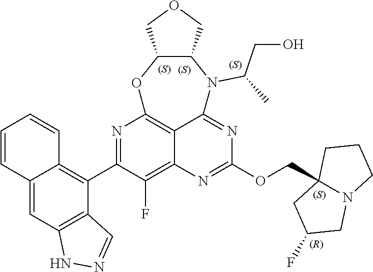 | 629.67 |
| 411 | 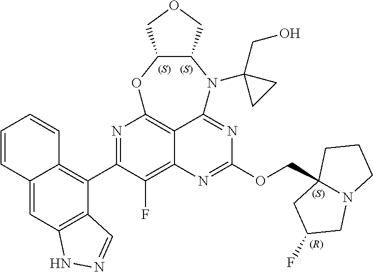 | 641.68 |
| 412 | 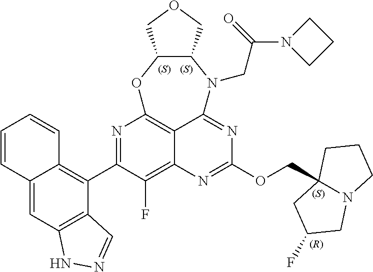 | 668.71 |
| 413 | 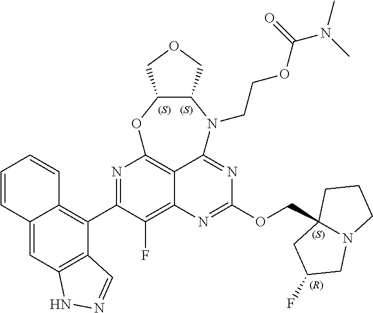 | 686.72 |
| 353 | 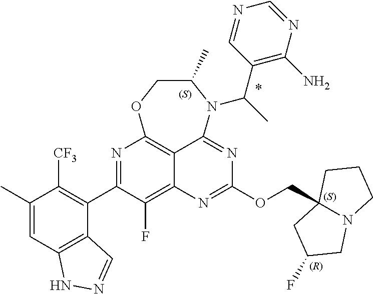 | 696.69 |
| 355 | 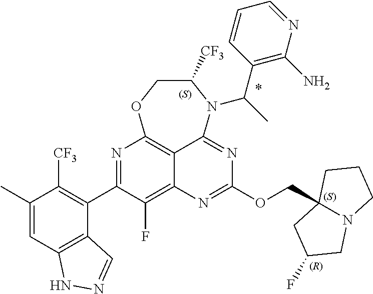 | 749.67 |
| 356 | 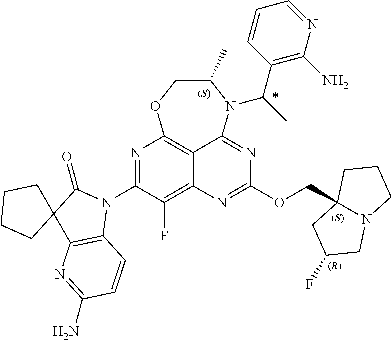 | 694.71 |
| 357 | 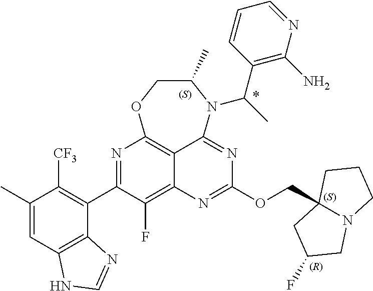 | 695.70 |
| 358 | 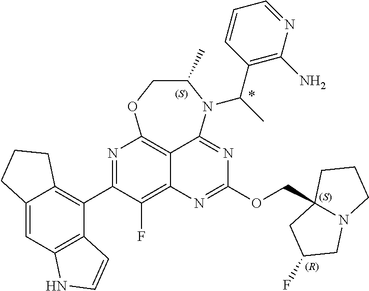 | 652.75 |
| 359 | 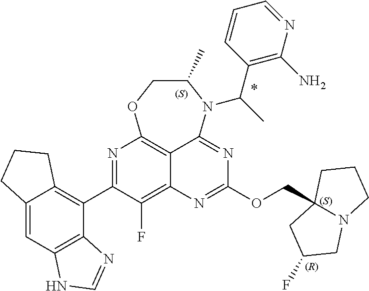 | 653.74 |
| 384 | 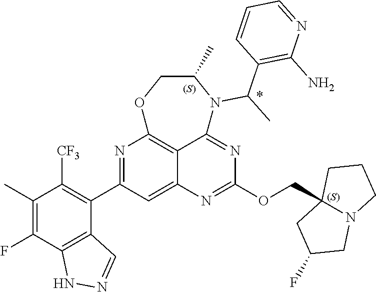 | 695.70 |
| 414 | 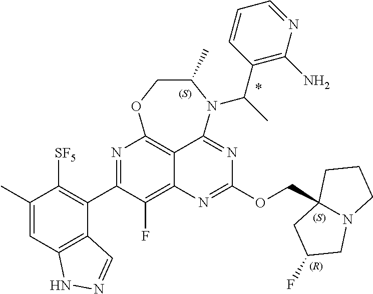 | 753.74 |
| 415 | 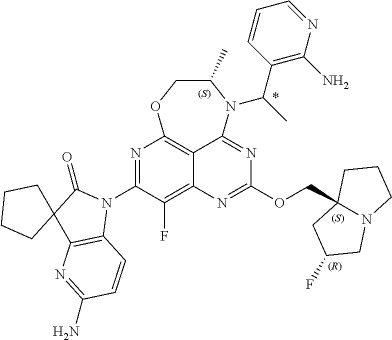 | 698.78 |
| 416 | 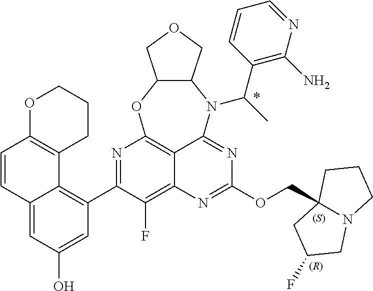 | 723.78 |
| 417 | 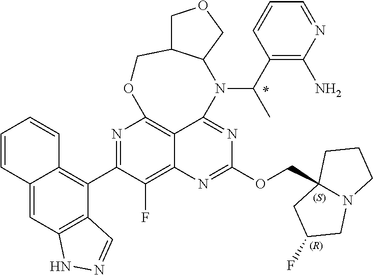 | 705.77 |
| 418 | 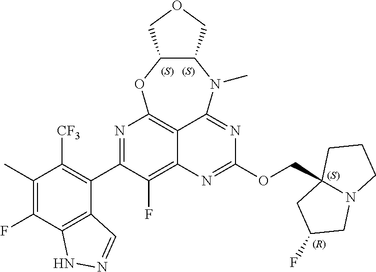 | 635.57 |
| 419 | 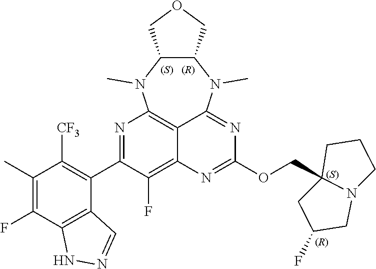 | 648.61 |
| 420 | 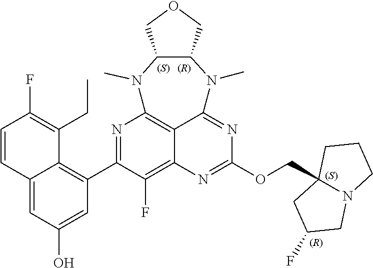 | 620.68 |
| 421 | 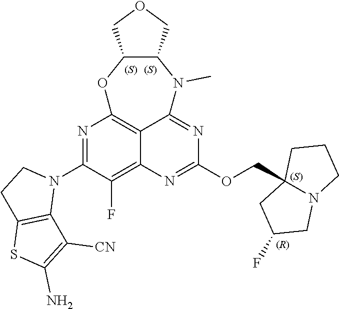 | 582.63 |
| 423 | 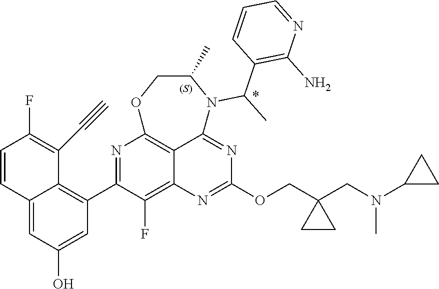 | 677.76 |
| 425 | 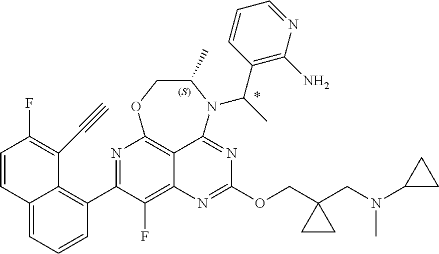 | 661.76 |
| 427 | 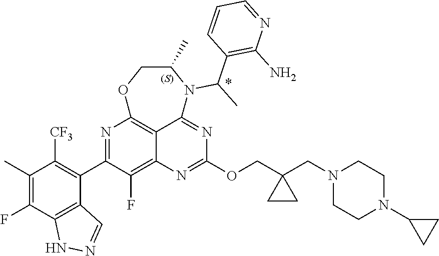 | 764.81 |
| 429 | 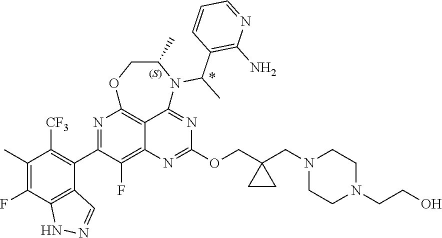 | 768.79 |
| 422 | 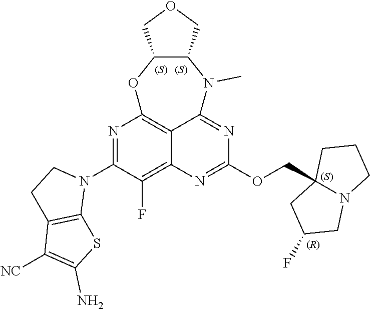 | 582.63 |
| 424 | 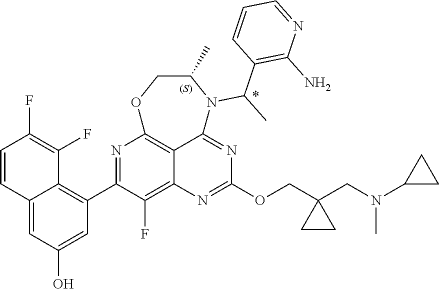 | 671.73 |
| 426 | 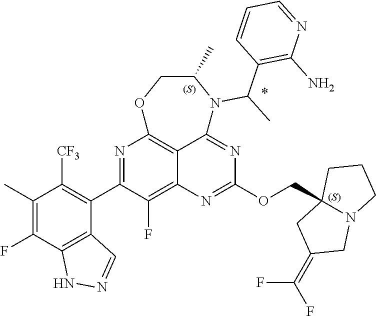 | 743.69 |
| 428 | 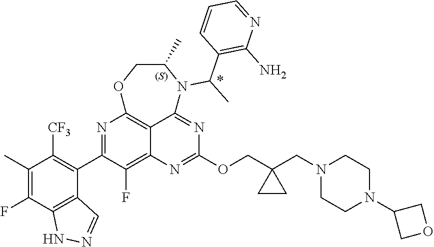 | 780.81 |
| 430 | 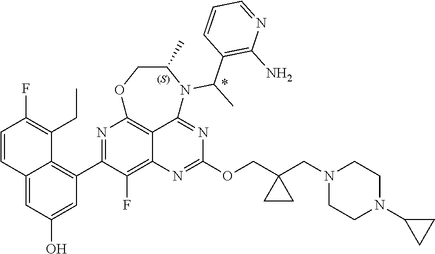 | 736.87 |

### Example 2: Assay of intracellular pERK1/2 (Thr202/Tyr204)phosphorylation

1. Reagents and materials:
pERK1/2 (Thr202/Tyr2O4) HIRE kit, Cisbio, Cat #64ERKPEH;
OptiPlate™-384-well plate, PerkinElmer, Cat #6007299;
96-well plate, Coming, Cat #353072;
Instrument: EnVision2104, PerkinElmer;
Cell lines: NCI-1H358 (KRAS G12C), ATCC, CRL-5807; PANC-1 (KRAS G121D), ATCC, CRL-1469; NCI-1H441 (KRAS G12V), ATCC, HTB-174.

### 2. Preparation of Reaction Solutions

A test compound was dissolved in DMSO, diluted to 200.0 μM and further diluted 3-fold to prepare a series of test compounds at concentrations of 66.7, 22.2, 7.4, 2.5, 0.82, 0.27, 0.09 μM etc. Then 10 μL of the compound at different dilution concentrations was added to 190 μL of a DMEM medium to prepare a 10× test compound;
1× cell lysis buffer: 4× cell lysis stock solution (provided by the kit) was diluted 4-fold with deionized water, and then 10% 100× blocking stock solution (provided by the kit) was added thereto.
pERK1/2 detection solution (prepared just before use): the pERK1/2 d₂ antibody (provided by the kit) and pERK1/2 Cryptate antibody (provided by the kit) were diluted with the detection solution (provided by the kit) at a ratio of 1:1:38.

### 3. Experimental Steps

Cells were inoculated into a 96-well plate (100 μL/well) at a density of 10000-12000/well. No cell was inoculated into a negative control well, and only the cell medium was added at 100 μL/well. The plate was placed in a cell incubator at 5% CO₂ and 37° C. overnight.
10 μL of the 10× test compound was added to the 100 μL cell culture 96-well plate; both the positive control well (that is, the control hole without chemical compound treatment) and the negative control well were charged with 10 μL of a 5% DMSO culture solution; and the plate was cultured in a cell incubator at 5% CO₂ and 37° C. for 2 days.
The medium in the 96-well plate was removed; 50 μL of the 1× cell lysis buffer was added to each of the wells; the plate was placed in a microplate shaker; and lysis under shaking was carried out at 900 rpm at room temperature for 1 hour.
16 μL of the lysis buffer in the 96-well plate was taken and transferred to a 384-well plate, followed by centrifugation at 1000 rpm for 30 s; then 4 μL of pERK1/2 detection solution was added to each well, followed by centrifugation at 1000 rpm for 30 s; and the 384-well plate was sealed with a sealing membrane and incubated at 25° C. on a low-speed shaker at 100 rpm in the dark for 2 hours.
The fluorescence value (RFU) of each well was detected on EnVision2104, with an emission wavelength 1 being 665 nm and an emission wavelength 2 being 615 nm.

### 4. Data Analysis

Fluorescence ratio=fluorescence value_{665 nm}/fluorescence value₆₁₅ nm

Inhibition rate (%)=100−((fluorescence ratio of compound well−fluorescence ratio of negative control well)/(fluorescence ratio of positive control well−fluorescence ratio of negative control well))×100

wherein:
the fluorescence ratio of compound well refers to the fluorescence ratio of the well containing the test compounds;
the fluorescence ratio of negative control well refers to the fluorescence ratio of the cell-free control well;
the fluorescence ratio of positive control well refers to the fluorescence ratio of the cell well treated with 0.5% DMSO.

IC₅₀ value: obtained by calculation using XL-Fit 5.0 software.

### 5. Test Results

| Compound | NCI-H358 (KRAS | PANC-1 (KRAS | NCI-H441 (KRAS |
|---|---|---|---|
| no. | G12C) IC₅₀ (μM) | G12D) IC₅₀ (μM) | G12V) IC₅₀ (μM) |
| 1 | 0.0007 | 0.006 | 0.003 |
| 2 | 0.009 | 0.321 | 0.042 |
| 3 | >1 | >1 | >1 |
| 4 | 0.001 | 0.010 | 0.002 |
| 5 | 0.710 | >1 | >1 |
| 6 | 0.004 | 0.136 | 0.017 |
| 7 | 0.008 | 0.238 | 0.033 |
| 8 | 0.001 | 0.113 | 0.002 |
| 9 | >1 | >1 | >1 |
| 10 | 0.152 | >1 | 0.797 |
| 11 | 0.001 | 0.004 | 0.001 |
| 12 | 0.054 | >1 | 0.192 |
| 13 | 0.137 | >1 | 0.247 |
| 14 | 0.019 | >1 | 0.036 |
| 15 | 0.002 | 0.175 | 0.017 |
| 16 | 0.002 | 0.151 | 0.005 |
| 17 | 0.029 | >1 | 0.108 |
| 18 | 0.001 | 0.054 | 0.003 |
| 19 | 0.015 | >1 | 0.066 |
| 20 | 0.783 | >1 | >1 |
| 21 | 0.0005 | 0.082 | 0.003 |
| 22 | 0.038 | 0.393 | 0.105 |
| 23 | >1 | >1 | >1 |
| 24 | 0.003 | 0.290 | 0.011 |
| 25 | 0.007 | 0.593 | 0.014 |
| 26 | 0.886 | >1 | >1 |
| 27 | 0.002 | 0.106 | 0.011 |
| 28 | 0.448 | >1 | >1 |
| 29 | >1 | >1 | >1 |
| 30 | 0.031 | >1 | 0.064 |
| 31 | 0.405 | >1 | >1 |
| 32 | 0.003 | 0.050 | 0.006 |
| 33 | 0.193 | >1 | 0.121 |
| 34 | >1 | >1 | >1 |
| 35 | 0.018 | 0.399 | 0.037 |
| 36 | 0.006 | 0.286 | 0.025 |
| 37 | 0.011 | 0.851 | 0.026 |
| 38 | 0.595 | >1 | 0.643 |
| 39 | 0.002 | 0.237 | 0.007 |
| 40 | 0.018 | 0.934 | 0.031 |
| 41 | 0.223 | >1 | 0.820 |
| 42 | 0.0009 | 0.069 | 0.005 |
| 43 | >1 | >1 | >1 |
| 44 | 0.0001 | 0.003 | 0.0006 |
| 45 | 0.038 | >1 | 0.053 |
| 46 | 0.004 | 0.287 | 0.005 |
| 47 | >1 | 0.347 | >1 |
| 48 | >1 | >1 | >1 |
| 49 | 0.015 | 0.557 | 0.022 |
| 50 | 0.916 | >1 | >1 |
| 51 | 0.002 | 0.100 | 0.005 |
| 53 | 0.088 | >1 | 0.191 |
| 54 | 0.0007 | 0.021 | 0.002 |
| 55 | 0.322 | >1 | 0.511 |
| 56 | 0.003 | 0.134 | 0.005 |
| 57 | 0.015 | 0.746 | 0.032 |
| 58 | 0.003 | 0.147 | 0.005 |
| 59 | 0.0002 | 0.002 | 0.0007 |
| 60 | 0.518 | >1 | 0.806 |
| 61 | 0.0002 | 0.0006 | 0.0002 |
| 62 | 0.008 | 0.055 | 0.009 |
| 63 | 0.0004 | 0.002 | 0.0005 |
| 64 | 0.0006 | 0.023 | 0.002 |
| 65 | 0.001 | 0.026 | 0.002 |
| 66 | 0.0002 | 0.003 | 0.0004 |
| 67 | 0.005 | 0.081 | 0.008 |
| 68 | 0.264 | >1 | 0.506 |
| 69 | 0.003 | 0.095 | 0.010 |
| 70 | 0.002 | >0.1 | 0.002 |
| 71 | 0.0001 | 0.0006 | 0.0004 |
| 72 | 0.011 | 0.038 | 0.013 |
| 73 | 0.0009 | 0.031 | 0.002 |
| 74 | 0.0002 | 0.004 | 0.0004 |
| 75 | 0.005 | 0.040 | 0.011 |
| 76 | 0.0005 | 0.018 | 0.0007 |
| 77 | 0.038 | >0.1 | 0.037 |
| 78 | 0.002 | 0.042 | 0.003 |
| 79 | >0.1 | >0.1 | >0.1 |
| 80 | 0.0005 | 0.019 | 0.0007 |
| 81 | >0.1 | >0.1 | >0.1 |
| 82 | 0.0008 | 0.015 | 0.0008 |
| 83 | 0.0006 | 0.029 | 0.003 |
| 85 | 0.0004 | 0.011 | 0.002 |
| 86 | 0.029 | >0.1 | 0.043 |
| 87 | 0.003 | 0.036 | 0.006 |
| 88 | 0.0004 | 0.003 | 0.001 |
| 89 | 0.026 | >0.1 | 0.043 |
| 90 | 0.002 | 0.091 | 0.006 |
| 91 | >0.1 | >0.1 | >0.1 |
| 92 | 0.001 | 0.058 | 0.002 |
| 93 | >0.1 | >0.1 | >0.1 |
| 94 | 0.046 | >0.1 | 0.100 |
| 95 | 0.0008 | 0.024 | 0.002 |
| 96 | 0.027 | >0.1 | 0.057 |
| 97 | 0.016 | >0.1 | 0.036 |
| 98 | 0.0003 | 0.010 | 0.0005 |
| 99 | 0.027 | >0.1 | 0.033 |
| 100 | 0.093 | >0.1 | >0.1 |
| 101 | >0.1 | >0.1 | >0.1 |
| 102 | 0.0008 | 0.038 | 0.002 |
| 103 | 0.060 | >0.1 | >0.1 |
| 104 | 0.002 | 0.024 | 0.003 |
| 105 | 0.0004 | 0.002 | 0.0006 |
| 106 | >0.1 | >0.1 | >0.1 |
| 107 | 0.089 | >0.1 | >0.1 |
| 108 | 0.012 | >0.1 | 0.026 |
| 109 | 0.0009 | 0.041 | 0.002 |
| 110 | >0.1 | >0.1 | >0.1 |
| 111 | 0.001 | 0.021 | 0.001 |
| 112 | 0.001 | 0.034 | 0.002 |
| 113 | 0.0007 | 0.002 | 0.0008 |
| 114 | 0.0006 | 0.019 | 0.0009 |
| 115 | 0.0002 | 0.0009 | 0.0002 |
| 116 | 0.004 | 0.023 | 0.009 |
| 117 | 0.026 | >0.1 | 0.036 |
| 118 | >0.1 | >0.1 | >0.1 |
| 119 | 0.0003 | 0.002 | 0.0004 |
| 120 | 0.0003 | 0.001 | 0.0004 |
| 121 | >0.1 | >0.1 | 0.098 |
| 122 | 0.0006 | 0.011 | 0.001 |
| 123 | 0.002 | >0.1 | 0.004 |
| 124 | 0.0001 | 0.001 | 0.0003 |
| 125 | 0.012 | 0.057 | 0.040 |
| 126 | 0.007 | >0.1 | 0.031 |
| 127 | >0.1 | >0.1 | >0.1 |
| 128 | >0.1 | >0.1 | >0.1 |
| 129 | 0.0009 | 0.026 | 0.002 |
| 130 | >0.1 | >0.1 | >0.1 |
| 131 | 0.0005 | 0.007 | 0.001 |
| 132 | 0.0004 | 0.014 | 0.001 |
| 133 | 0.003 | >0.1 | 0.010 |
| 134 | 0.031 | >0.1 | 0.034 |
| 135 | 0.002 | 0.030 | 0.003 |
| 136 | 0.017 | >0.1 | 0.061 |
| 137 | 0.0001 | 0.0009 | 0.0002 |
| 138 | 0.013 | 0.030 | 0.017 |
| 139 | 0.091 | >0.1 | >0.1 |
| 140 | 0.0003 | 0.001 | 0.0009 |
| 141 | 0.0003 | >0.1 | 0.0007 |
| 142 | >0.1 | >0.1 | >0.1 |
| 143 | 0.0005 | 0.021 | 0.002 |
| 144 | 0.0004 | 0.017 | 0.002 |
| 145 | 0.014 | >0.1 | 0.051 |
| 146 | 0.0008 | 0.016 | 0.003 |
| 148 | 0.0002 | 0.001 | 0.0003 |
| 149 | 0.0003 | 0.007 | 0.0009 |
| 150 | 0.0002 | 0.001 | 0.0003 |
| 153 | 0.0002 | 0.002 | 0.0005 |
| 154 | 0.021 | >0.1 | 0.031 |
| 157 | 0.0003 | 0.002 | 0.0005 |
| 158 | 0.096 | >0.1 | >0.1 |
| 159 | 0.0006 | 0.015 | 0.001 |
| 160 | 0.0005 | >0.1 | 0.002 |
| 161 | 0.014 | >0.1 | 0.048 |
| 162 | >0.1 | >0.1 | >0.1 |
| 163 | 0.0002 | 0.006 | 0.0004 |
| 164 | 0.0002 | 0.003 | 0.0003 |
| 165 | 0.001 | 0.047 | 0.003 |
| 166 | 0.069 | >0.1 | >0.1 |
| 167 | >0.1 | >0.1 | >0.1 |
| 168 | 0.0002 | 0.002 | 0.0004 |
| 169 | 0.0007 | 0.017 | 0.002 |
| 170 | 0.0002 | 0.002 | 0.0005 |
| 171 | 0.0005 | 0.027 | 0.002 |
| 173 | 0.0005 | 0.007 | 0.0008 |
| 174 | 0.0002 | 0.010 | 0.0006 |
| 177 | 0.0007 | 0.011 | 0.002 |
| 178 | 0.0001 | 0.001 | 0.0004 |
| 179 | 0.001 | 0.020 | 0.003 |
| 180 | 0.001 | 0.035 | 0.006 |
| 181 | >0.1 | >0.1 | >0.1 |
| 183 | 0.0001 | 0.001 | 0.0003 |
| 184 | 0.005 | 0.028 | 0.013 |
| 185 | 0.0003 | 0.013 | 0.001 |
| 187 | 0.063 | >0.1 | 0.044 |
| 195 | 0.033 | >0.1 | 0.099 |
| 196 | >0.1 | >0.1 | >0.1 |
| 197 | >0.1 | >0.1 | >0.1 |
| 198 | >0.1 | >0.1 | >0.1 |
| 199 | 0.0002 | 0.001 | 0.0002 |
| 200 | 0.007 | 0.063 | 0.010 |
| 202 | 0.0004 | 0.013 | 0.001 |
| 203 | 0.088 | >0.1 | >0.1 |
| 204 | 0.001 | 0.049 | 0.004 |
| 205 | 0.0003 | 0.011 | 0.001 |
| 206 | 0.0002 | 0.0008 | 0.0003 |
| 207 | 0.009 | >0.1 | 0.033 |
| 208 | 0.0004 | 0.010 | 0.001 |
| 209 | 0.013 | >0.1 | 0.045 |
| 210 | 0.0007 | 0.006 | 0.002 |
| 211 | 0.048 | >0.1 | >0.1 |
| 212 | 0.0003 | 0.006 | 0.0006 |
| 213 | 0.013 | >0.1 | 0.018 |
| 214 | 0.0002 | 0.004 | 0.0003 |
| 215 | 0.021 | >0.1 | 0.036 |
| 216 | 0.002 | 0.048 | 0.002 |
| 217 | >0.1 | >0.1 | >0.1 |
| 218 | 0.0005 | 0.004 | 0.001 |
| 219 | 0.0004 | 0.007 | 0.001 |
| 220 | 0.006 | >0.1 | 0.021 |
| 221 | 0.0001 | 0.002 | 0.0004 |
| 222 | 0.0001 | 0.002 | 0.0004 |
| 223 | 0.002 | 0.020 | 0.007 |
| 224 | >0.1 | >0.1 | >0.1 |
| 225 | 0.0002 | 0.0005 | 0.0005 |
| 226 | 0.043 | >0.1 | >0.1 |
| 227 | 0.0002 | 0.002 | 0.0006 |
| 228 | 0.0002 | 0.0008 | 0.0005 |
| 229 | 0.0001 | 0.0005 | 0.0005 |
| 230 | 0.0001 | 0.001 | 0.0005 |
| 231 | 0.0002 | 0.0008 | 0.0002 |
| 232 | 0.0002 | 0.005 | 0.0009 |
| 233 | 0.001 | 0.032 | 0.003 |
| 237 | 0.0003 | 0.001 | 0.0004 |
| 238 | 0.0002 | 0.0008 | 0.0006 |
| 239 | 0.0003 | 0.002 | 0.0004 |
| 241 | 0.0002 | 0.013 | 0.0009 |
| 242 | 0.0004 | >0.1 | 0.003 |
| 243 | 0.0002 | 0.001 | 0.0005 |
| 245 | 0.0002 | 0.010 | 0.0005 |
| 246 | 0.0004 | 0.063 | 0.002 |
| 247 | >0.1 | >0.1 | >0.1 |
| 248 | 0.0004 | 0.008 | 0.001 |
| 250 | 0.0001 | 0.0004 | 0.0003 |
| 251 | >0.1 | >0.1 | >0.1 |
| 252 | >0.1 | >0.1 | >0.1 |
| 253 | >0.1 | >0.1 | >0.1 |
| 254 | 0.002 | 0.043 | 0.010 |
| 255 | 0.037 | >0.1 | >0.1 |
| 256 | >0.1 | >0.1 | >0.1 |
| 257 | 0.002 | 0.051 | 0.006 |
| 258 | >0.1 | >0.1 | >0.1 |
| 259 | 0.0009 | 0.001 | 0.001 |
| 260 | 0.030 | >0.1 | 0.065 |
| 261 | >0.1 | >0.1 | >0.1 |
| 262 | 0.028 | 0.040 | 0.047 |
| 263 | 0.014 | 0.035 | 0.040 |
| 264 | 0.093 | >0.1 | >0.1 |
| 265 | >0.1 | >0.1 | >0.1 |
| 266 | 0.095 | >0.1 | >0.1 |
| 267 | 0.0003 | 0.0002 | 0.0006 |
| 268 | 0.0002 | 0.0004 | 0.0005 |
| 269 | 0.0002 | 0.0006 | 0.0003 |
| 270 | 0.0003 | 0.0003 | 0.0007 |
| 271 | 0.066 | >0.1 | >0.1 |
| 272 | 0.057 | >0.1 | >0.1 |
| 273 | 0.0005 | 0.0004 | 0.001 |
| 274 | 0.005 | 0.013 | 0.008 |
| 275 | 0.0004 | 0.0004 | 0.0008 |
| 276 | 0.077 | >0.1 | >0.1 |
| 277 | 0.093 | >0.1 | >0.1 |
| 278 | 0.0002 | 0.002 | 0.0006 |
| 279 | 0.0003 | 0.0002 | 0.0004 |
| 280 | 0.006 | 0.005 | 0.011 |
| 281 | 0.0002 | 0.001 | 0.0005 |
| 282 | 0.0002 | 0.003 | 0.0004 |
| 283 | 0.055 | >0.1 | >0.1 |
| 284 | 0.0002 | 0.0003 | 0.0005 |
| 285 | 0.027 | 0.086 | 0.017 |
| 286 | 0.057 | >0.1 | >0.1 |
| 287 | 0.0002 | 0.002 | 0.0004 |
| 289 | 0.0003 | 0.004 | 0.001 |
| 290 | 0.0002 | 0.0002 | 0.0003 |
| 291 | 0.0001 | 0.001 | 0.0003 |
| 293 | 0.001 | 0.0005 | 0.001 |
| 294 | 0.0008 | 0.0005 | 0.002 |
| 295 | 0.0004 | 0.0005 | 0.001 |
| 296 | 0.0005 | 0.0003 | 0.002 |
| 297 | 0.0005 | 0.003 | 0.001 |
| 298 | >0.1 | >0.1 | >0.1 |
| 300 | 0.0006 | 0.0005 | 0.002 |
| 301 | 0.025 | 0.077 | 0.072 |
| 302 | >0.1 | >0.1 | >0.1 |
| 304 | 0.021 | 0.055 | 0.046 |
| 305 | 0.0003 | 0.0003 | 0.0006 |
| 306 | 0.016 | 0.093 | 0.040 |
| 307 | 0.013 | 0.016 | 0.023 |
| 310 | >0.1 | >0.1 | >0.1 |
| 311 | 0.0005 | 0.006 | 0.0008 |
| 312 | 0.0003 | 0.0003 | 0.0004 |
| 313 | 0.0005 | 0.003 | 0.001 |
| 314 | >0.1 | >0.1 | >0.1 |
| 315 | 0.002 | 0.006 | 0.004 |
| 316 | >0.1 | >0.1 | >0.1 |
| 317 | 0.0004 | 0.0003 | 0.0008 |
| 318 | 0.001 | 0.037 | 0.007 |
| 319 | 0.0009 | 0.010 | 0.001 |
| 320 | 0.0009 | 0.028 | 0.004 |
| 321 | 0.001 | 0.036 | 0.007 |
| 322 | 0.021 | >0.1 | 0.097 |
| 323 | 0.0006 | 0.010 | 0.002 |
| 324 | 0.028 | >0.1 | >0.1 |
| 325 | 0.028 | >0.1 | 0.044 |
| 326 | 0.001 | 0.003 | 0.003 |
| 327 | >0.1 | >0.1 | >0.1 |
| 328 | 0.0005 | 0.0008 | 0.0008 |
| 329 | >0.1 | >0.1 | >0.1 |
| 330 | 0.0004 | 0.0003 | 0.0005 |
| 331 | 0.038 | >0.1 | >0.1 |
| 332 | 0.0004 | 0.0003 | 0.0009 |
| 333 | 0.001 | 0.006 | 0.001 |
| 334 | 0.0003 | 0.0006 | 0.0005 |
| 335 | >0.1 | >0.1 | >0.1 |
| 336 | >0.1 | >0.1 | >0.1 |
| 337 | >0.1 | >0.1 | >0.1 |
| 338 | 0.023 | >0.1 | 0.049 |
| 339 | >0.1 | >0.1 | >0.1 |
| 340 | 0.002 | 0.039 | 0.004 |
| 342 | 0.0002 | 0.0003 | 0.0004 |
| 343 | 0.0002 | 0.002 | 0.0006 |
| 348 | 0.0002 | 0.0005 | 0.0005 |
| 360 | >0.1 | >0.1 | >0.1 |
| 361 | 0.002 | 0.034 | 0.005 |
| 362 | 0.0002 | 0.0004 | 0.0003 |
| 363 | 0.0002 | 0.0003 | 0.0003 |
| 364 | >0.1 | >0.1 | >0.1 |
| 365 | 0.0002 | 0.001 | 0.001 |
| 366 | >0.1 | >0.1 | >0.1 |
| 367 | 0.0002 | 0.001 | 0.0004 |
| 368 | 0.0003 | 0.0006 | 0.0003 |
| 369 | 0.0002 | 0.0004 | 0.0002 |
| 370 | 0.001 | 0.002 | 0.003 |
| 371 | 0.003 | 0.057 | 0.011 |
| 372 | 0.0003 | 0.004 | 0.0008 |
| 373 | 0.014 | 0.041 | 0.025 |
| 374 | 0.0006 | 0.001 | 0.002 |
| 375 | 0.008 | >0.1 | 0.094 |
| 376 | 0.0004 | 0.004 | 0.001 |
| 377 | 0.002 | 0.002 | 0.005 |
| 378 | 0.0002 | 0.0002 | 0.0006 |
| 379 | 0.0002 | 0.0001 | 0.0003 |
| 380 | 0.001 | 0.001 | 0.002 |
| 381 | 0.0002 | 0.0002 | 0.0004 |
| 382 | 0.003 | 0.002 | 0.005 |
| 383 | 0.0005 | 0.0004 | 0.0004 |
| 385 | 0.0007 | 0.0004 | 0.002 |
| 386 | 0.0004 | 0.006 | 0.0007 |
| 387 | 0.002 | 0.017 | 0.005 |
| 388 | >0.1 | >0.1 | >0.1 |
| 389 | 0.060 | >0.1 | >0.1 |
| 390 | 0.011 | >0.1 | 0.031 |
| 391 | >0.1 | >0.1 | >0.1 |
| 392 | >0.1 | >0.1 | >0.1 |
| 393 | >0.1 | >0.1 | >0.1 |
| 394 | 0.001 | 0.036 | 0.002 |
| 395 | 0.001 | 0.021 | 0.004 |
| 396 | <0.000457247 | 0.006 | 0.001 |
| 397 | <0.000457247 | 0.0007 | 0.0008 |
| 398 | 0.212 | 0.264 | 0.472 |
| 399 | 0.065 | 0.403 | 0.057 |
| 400 | 0.676 | 0.831 | >1 |
| 401 | 0.0003 | 0.0003 | 0.0004 |
| 402 | 0.0004 | 0.0004 | 0.0006 |
| 403 | 0.112 | 0.997 | 0.308 |

1. A compound of formula (I): 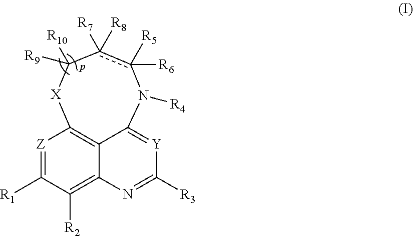
or a pharmaceutically acceptable salt thereof, or a solvate, a racemic mixture, an enantiomer, a diastereomer or a tautomer thereof, wherein
X is O, NR₁₁, CR₁₅R₁₆, C(O), S, S(O) or S(O)₂;
R₁₁ is independently selected from hydrogen, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, —CO(C₁₋₆ alkyl), —CONH₂, —CONH(C₁₋₆ alkyl), —CON(C₁₋₆ alkyl)₂, C₃₋₈ cycloalkyl, and 3-8 membered heterocyclyl;
R₁₅ and R₁₋₆ are each independently selected from hydrogen, deuterium, halogen, —CN, —OH, —NH₂, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O(C₁₋₆ alkyl), —O(C₁₋₆ haloalkyl), —NH(C₁₋₆ alkyl), —N(C₁₋₆ alkyl)₂, —CO(C₁₋₆ alkyl), —CONH₂, —CONH(C₁₋₆ alkyl), —CON(C₁₋₆ alkyl)₂, C₃₋₈ cycloalkyl, and 3-8 15 membered heterocyclyl; or R₁₅ and R₁₋₆ together with the carbon atom to which they are attached form C₃₋₆ cycloalkyl or 3-6 membered heterocyclyl;
Y is N, and Z is N; or Y is C—CN, and Z is N or Z is CR₂; [symbol]
represents a double bond or a single bond, and when [symbol] represents a double bond R₆ and R₈ are absent;
p is 0, 1 or 2;
R₁ is —L₁—R₁₈;
L₁ is absent, or L₁ is NR₁, O, C(O), S, S(O) or S(O)₂;
R₁₈ is selected from 6-14 membered aryl, 5-14 membered heteroaryl, C₃₋₁₀ cycloalkyl, and 4-14 membered heterocyclyl, each of which is optionally substituted with one or more groups independently selected from: halogen, —CN, —OH, —SH, —SF₅, oxo, C₁₋₆ alkyl, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —O(C₃₋₈ cycloalkyl), —O(3-8 membered heterocyclyl), —S(C₁₋₆ alkyl), —S(C₁₋₆ haloalkyl), —S(C₃₋₈ cycloalkyl), —S(3-8 membered heterocyclyl), —OC(O)NRₐR_{b}, —NRₐC(O)OR_{c}, —NRₐC(O)R_{d}, —NRₐR_{b}, —C(O)NRₐR_{b}, —C(O)OR_{c}, —C(O)R_{d}, —S(O)₂R_{f}, and —S(O)₂NRₐR_{b}; wherein the C₁₋₆ alkyl is optionally substituted with one or more deuterium, and the C₂₋₆ alkenyl, C₂₋₆ alkynyl, C₃₋₈ cycloalkyl and 3-8 membered heterocyclyl are each optionally substituted with one or more halogen;
R₂ is selected from hydrogen, halogen, —CN, —OH, —SH, oxo, C₁₋₆ alkyl, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —O(C₃₋₈ cycloalkyl), —O(3-8 membered heterocyclyl), —S(C₁₋₆ alkyl), —S(C₁₋₆ haloalkyl), —S(C₃₋₈ cycloalkyl), —S(3-8 membered heterocyclyl), —OC(O)NRₐR_{b}, —NRₐC(O)ORₑ, —NRₐC(O)R_{d}, —NRₐR_{b}, —C(O)NRₐR_{b}, —C(O)OR_{c}, —C(O)R_{d}, —S(O)₂R_{f}, and —S(O)₂NRₐR_{b}; wherein the C₁₋₆ alkyl is optionally substituted with one or more deuterium, and the C₂₋₆ alkenyl, C₂₋₆ alkynyl, C₃₋₈ cycloalkyl and 3-8 membered heterocyclyl are each optionally substituted with one or more halogen;
R₃ is selected from hydrogen, —OH, —SH, —NH₂, C₁₋₆ alkyl, C₁₋₆ haloalkyl, —O(C₁₋₆ alkyl), —O(C₁₋₆ haloalkyl), —S(C₁₋₆ alkyl), —S(C₁₋₆ haloalkyl), —NH(C₁₋₆ alkyl), —N(C₁₋₆ alkyl)₂, C₃₋₈ cycloalkyl, 4-10 membered heterocyclyl, and —O—L₂—R₁₂; wherein the C₃₋₈ cycloalkyl and 4-10 membered heterocyclyl are each optionally substituted with one or more groups independently selected from: deuterium, halogen, —CN, —OH, —SH, oxo, —NH₂, C₁₋₆ alkyl, C₁₋₆ alkylidene, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), and —S(C₁₋₆ alkyl), wherein the C₁₋₆ alkylidene is optionally substituted with one or more halogen;
L₂ is absent, or L₂ is C₁₋₆ alkylene or C₃₋₈ cycloalkylene, wherein the C₁₋₆ alkylene and C₃₋₈ cycloalkylene are each optionally substituted with one or more deuterium or halogen;
R₁₂ is C₃₋₁₀ cycloalkyl or 4-12 membered heterocyclyl, each of which is optionally substituted with one or more groups independently selected from: deuterium, halogen, —CN, —OH, —SH, oxo, C₁₋₆ alkyl, C₁₋₆ alkylidene, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-O—(C₁₋₆ alkyl), —O(C₁₋₆ alkyl), —O(C₁₋₆haloalkyl), —O(C₃₋₈ cycloalkyl), —O(3-8 membered heterocyclyl), —S(C₁₋₆ alkyl), —S(C₁₋₆ haloalkyl), —S(C₃₋₈ cycloalkyl), —S(3-8 membered heterocyclyl), —OC(O)NRₐR_{b}, —NRₐC(O)ORₑ, —NRₐC(O)R_{d}, —NRₐR_{b}, —C(O)NRₐR_{b}, —C(O)OR_{d}, —C(O)R_{d}, —S(O)₂R_{f}, and —S(O)₂NRₐR_{b}; wherein the C₁₋₆ alkyl, C₁₋₆ alkylidene, C₂₋₆ alkenyl, C₂₋₆ alkynyl, C₃₋₈ cycloalkyl and 3-8 membered heterocyclyl are each optionally substituted with one or more R₁₇;
R₁₇ is selected from deuterium, halogen, —CN, —OH, —SH, oxo, C₁₋₆ alkyl, C₁₋₆ alkylidene, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —S(C₁₋₆ alkyl), —S(C₁₋₆ haloalkyl), —NRₐR_{b}, —NHCO(C₁₋₆ alkyl), —CO(C₁₋₆ alkyl), —CONH₂, —CONH(C₁₋₆ alkyl), —CON(C₁₋₆ alkyl)₂, —S(O)₂(C₁₋₆ alkyl), —S(O)₂NH(C₁₋₆ alkyl), and —S(O)₂N(C₁₋₆ alkyl)₂; wherein the 3-8 membered heterocyclyl is optionally substituted with one or more groups independently selected from: deuterium, halogen, —CN, —OH, oxo, —NH₂, C₁₋₆ alkyl, C₁₋₆ alkylidene, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —S(C₁₋₆ alkyl), —S(C₁₋₆ haloalkyl), —CO(C₁₋₆ alkyl), and —S(O)₂(C₁₋₆ alkyl);
R₄ is selected from C₁₋₆ alkyl, C₂₋₆ alkenyl, C₃₋₁₀ cycloalkyl, 4-12 membered heterocyclyl and 5-12 membered heteroaryl, each of which is optionally substituted with one or more R₁₃;
R₁₃ is selected from deuterium, halogen, —CN, —OH, —SH, oxo, —NH₂, C₁₋₆ alkyl, C₂₋₆ alkenyl, C₂₋₆ alkynyl, C₁₋₆ haloalkyl, C₃₋₈ cycloalkyl, 3-8 membered heterocyclyl, 6-10 membered aryl, 5-10 membered heteroaryl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —S(C₁₋₆ alkyl), —S(C₁₋₆ haloalkyl), —NHCO(C₁₋₆ alkyl), —NH(C₁₋₆ alkyl), —N(C₁₋₆ alkyl)₂, —CO(C₁₋₆ alkyl), —CONH₂, —CONH(C₁₋₆ alkyl), —CON(C₁₋₆ alkyl)₂, —S(O)₂(C₁₋₆ alkyl), —S(O)₂NH(C₁₋₆ alkyl), and —S(O)₂N(C₁₋₆ alkyl)₂; wherein the C₃₋₈ cycloalkyl, 3-8 membered heterocyclyl, 6-10 membered aryl and 5-10 membered heteroaryl are each optionally substituted with one or more R₁₄;
R₁₄ is independently selected from deuterium, halogen, —CN, —OH, —SH, oxo, C₁₋₆ alkyl, C₁₋₆ haloalkyl, C₃₋₈ cycloalkyl, 3-8 membered heterocyclyl, phenyl, 5-6 membered heteroaryl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl)-, —O(C₃₋₈ cycloalkyl), —O(3-8 membered heterocyclyl), —S(C₁₋₆ alkyl), —S(C₁₋₆ haloalkyl), —S(C₃₋₈ cycloalkyl), —S(3-8 membered heterocyclyl), —NRₐC(O)R_{d}, —NRₐR_{b}, —C(O)NRₐR_{b}, —C(O)OR_{c}, —C(O)R_{d}, —S(O)₂R_{f}, and —S(O)₂NRₐR_{b}; wherein the C₁₋₆ alkyl is optionally substituted with one or more deuterium, and the C₃₋₈ cycloalkyl, 3-8 membered heterocyclyl, phenyl and 5-6 membered heteroaryl are each optionally substituted with one or more halogen;
R₅, R₆, R₇, R₈, R₉ and R₁₀ are each independently selected from hydrogen, deuterium, halogen, —CN, —OH, —SH, oxo, —NH₂, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O(C₁₋₆ alkyl), —O(C₁₋₆ haloalkyl), —NH(C₁₋₆ alkyl), —N(C₁₋₆ alkyl)₂, —CO(C₁₋₆ alkyl), —CONH₂, —CONH(C₁₋₆ alkyl), —CON(C₁₋₆ alkyl)₂, C₃₋₈ cycloalkyl, and 3-8 membered heterocyclyl; or R₅ and R₆, R₇ and R₈, R₉ and R₁₀, R₅ and R₇, R₇ and R₉, or R₅ and R₉ together with the carbon atoms to which they are attached form C₃₋₆ cycloalkyl or 3-6 membered heterocyclyl;
or R₄ and R₅ together with the nitrogen atom and carbon atom to which they are attached form 5-12 membered heteroaryl, which is optionally substituted with one or more R₁₄;
or R₄ and R₅ together with the nitrogen atom and carbon atom to which they are attached form 4-10 membered heterocyclyl; wherein the 4-10 membered heterocyclyl is substituted with one or more groups independently selected from: C₃₋₈ cycloalkyl, 3-8 membered heterocyclyl, 6-10 membered aryl, and 5-10 membered heteroaryl; and the 4-10 membered heterocyclyl is further optionally substituted with one or more groups independently selected from: deuterium, halogen, —CN, —OH, —SH, oxo, —NH₂, C₁₋₆ alkyl, C₂₋₆ alkenyl, C₂₋₆ alkynyl, C₁₋₆ haloalkyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —S(C₁₋₆ alkyl), —S(C₁₋₆ haloalkyl), —NHCO(C₁₋₆ alkyl), —NH(C₁₋₆ alkyl), —N(C₁₋₆ alkyl)₂, —CO(C₁₋₆ alkyl), —CONH₂, —CONH(C₁₋₆ alkyl), —CON(C₁₋₆ alkyl)₂, —S(O)₂(C₁₋₆ alkyl), —S(O)₂NH(C₁₋₆ alkyl), and —S(O)₂N(C₁₋₆ alkyl)₂; wherein the C₃₋₈ cycloalkyl, 3-8 membered heterocyclyl, 6-10 membered aryl and 5-10 membered heteroaryl are each optionally substituted with one or more R₁₄; and
Rₐ, R_{b}, R_{c}, R_{d} and R_{f} are each independently selected from hydrogen, C₁₋₆ alkyl, C₃₋₈ cycloalkyl, 3-8 membered heterocyclyl, phenyl, and 5-6 membered heteroaryl; wherein the C₁₋₆ alkyl, C₃₋₈ cycloalkyl, 3-8 membered heterocyclyl, phenyl and 5-6 membered heteroaryl are each optionally substituted with one or more groups independently selected from: halogen, —CN, —OH, —O(C₁₋₆ alkyl), C₃₋₈ cycloalkyl, 3-8 membered heterocyclyl, —NR_{g}Rₕ, —C(O)NR_{g}Rₕ, and —NR_{g}C(O)Rᵢ; wherein R_{g}, Rₕ and Rᵢ are each independently selected from hydrogen, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, —(C₁₋₆ alkyl)-NH₂, —(C₁₋₆ alkyl)-NH(C₁₋₆ alkyl), —(C₁₋₆ alkyl)-N(C₁-(alkyl)₂, C₃₋₈ cycloalkyl, 3-8 membered heterocyclyl, phenyl, and 5-6 membered heteroaryl; or Rₐ and R_{b} together with the nitrogen atom to which they are attached form 3-8 membered heterocyclyl.

2. The compound or the pharmaceutically acceptable salt thereof, or the solvate, the racemic mixture, the enantiomer, the diastereomer or the tautomer thereof according to claim 1, wherein
X is O, NR₁₁, CR₁₅R₁₋₆, C(O), S, S(O) or S(O)₂;
R₁₁ is independently selected from hydrogen, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, —CO(C₁₋₆ alkyl), —CONH₂, —CONH(C₁₋₆ alkyl), —CON(C₁₋₆ alkyl)₂, C₃₋₈ cycloalkyl, and 3-8 membered heterocyclyl;
R₁₅ and R₁₋₆ are each independently selected from hydrogen, deuterium, halogen, —CN, —OH, —NH₂, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O(C₁₋₆ alkyl), —O(C₁₋₆ haloalkyl), —NH(C₁₋₆ alkyl), —N(C₁₋₆ alkyl)₂, —CO(C₁₋₆ alkyl), —CONH₂, —CONH(C₁₋₆ alkyl), —CON(C₁₋₆ alkyl)₂, C₃₋₈ cycloalkyl, and 3-8 membered heterocyclyl; or R₁₅ and R₁₋₆ together with the carbon atom to which they are attached form C₃₋₆ cycloalkyl or 3-6 membered heterocyclyl;
Y is N, and Z is N; or Y is C—CN, and Z is N or Z is CR₂; [symbol]
represents a double bond or a single bond, and when [symbol] represents a double bond, R₆ and R₈ are absent;
p is 0, 1 or 2;
R₁ is —L₁—R₁₈;
L₁ is absent, or L₁ is NR₁₁, O, C(O), S, S(O) or S(O)₂;
R₁₈ is selected from 6-12 membered aryl, 5-12 membered heteroaryl, C₃₋₁₀ cycloalkyl, and 4-12 membered heterocyclyl, each of which is optionally substituted with one or more groups independently selected from: halogen, —CN, —OH, —SH, oxo, C₁₋₆ alkyl, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —O(C₃₋₈ cycloalkyl), —O(3-8 membered heterocyclyl), —S(C₁₋₆ alkyl), —S(C₁₋₆ haloalkyl), —S(C₃₋₈ cycloalkyl), —S(3-8 membered heterocyclyl), —OC(O)NRₐR_{b}, —NRₐC(O)OR_{c}, —NRₐC(O)R_{d}, —NRₐR_{b}, —C(O)NRₐR_{b}, —C(O)OR_{d}, —C(O)R_{d}, —S(O)₂R_{f}, and —S(O)₂NRₐR_{b}; wherein the C₁₋₆ alkyl is optionally substituted with one or more deuterium, and the C₂₋₆ alkenyl, C₂₋₆ alkynyl, C₃₋₈ cycloalkyl and 3-8 membered heterocyclyl are each optionally substituted with one or more halogen;
R₂ is selected from hydrogen, halogen, —CN, —OH, —SH, oxo, C₁₋₆ alkyl, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —O(C₃₋₈ cycloalkyl), —O(3-8 membered heterocyclyl), —S(C₁₋₆ alkyl), —S(C₁₋₆ haloalkyl), —S(C₃₋₈ cycloalkyl), —S(3-8 membered heterocyclyl), —OC(O)NRₐR_{b}, —NRₐC(O)OR_{d}, —NRₐC(O)R_{d}, —NRₐR_{b}, —C(O)NRₐR_{b}, —C(O)OR_{c}, —C(O)R_{d}, —S(O)₂R_{f}, and —S(O)₂NRₐR_{b}; wherein the C₁₋₆ alkyl is optionally substituted with one or more deuterium, and the C₂₋₆ alkenyl, C₂₋₆ alkynyl, C₃₋₈ cycloalkyl and 3-8 membered heterocyclyl are each optionally substituted with one or more halogen;
R₃ is selected from hydrogen, —OH, —SH, —NH₂, C₁₋₆ alkyl, C₁₋₆ haloalkyl, —O(C₁₋₆ alkyl), —O(C₁₋₆ haloalkyl), —S(C₁₋₆ alkyl), —S(C₁₋₆ haloalkyl), —NH(C₁₋₆ alkyl), —N(C₁₋₆ alkyl)₂, C₃₋₈ cycloalkyl, 4-10 membered heterocyclyl, and —O—L₂—R₁₂; wherein the C₃₋₈ cycloalkyl and 4-10 membered heterocyclyl are each optionally substituted with one or more groups independently selected from: deuterium, halogen, —CN, —OH, —SH, oxo, —NH₂, C₁₋₆ alkyl, C₁₋₆ alkylidene, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), and —S(C₁₋₆ alkyl), wherein the C₁₋₆ alkylidene is optionally substituted with one or more halogen;
L₂ is absent, or L₂ is C₁₋₆ alkylene or C₃₋₈ cycloalkylene, wherein the C₁₋₆ alkylene and C₃₋₈ cycloalkylene are each optionally substituted with one or more deuterium or halogen;
R₁₂ is C₃₋₁₀ cycloalkyl or 4-12 membered heterocyclyl, each of which is optionally substituted with one or more groups independently selected from: deuterium, halogen, —CN, —OH, —SH, oxo, C₁₋₆ alkyl, C₁₋₆ alkylidene, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-O—(C₁₋₆ alkyl), —O(C₁₋₆ alkyl), —O(C₁₋₆ haloalkyl), —O(C₃₋₈ cycloalkyl), —O(3-8 membered heterocyclyl), —S(C₁₋₆ alkyl), —S(C₁₋₆ haloalkyl), —S(C₃₋₈ cycloalkyl), —S(3-8 membered heterocyclyl), —OC(O)NRₐR_{b}, —NRₐC(O)OR_{c}, —NRₐC(O)R_{d}, —NRₐR_{b}, —C(O)NRₐR_{b}, —C(O)OR_{d}, —C(O)R_{d}, —S(O)₂R_{f}, and —S(O)₂NRₐRe; wherein the C₁₋₆ alkyl, C₁₋₆ alkylidene, C₂₋₆ alkenyl, C₂₋₆ alkynyl, C₃₋₈ cycloalkyl and 3-8 membered heterocyclyl are each optionally substituted with one or more R₁₇;
R₁₇ is selected from deuterium, halogen, —CN, —OH, —SH, oxo, C₁₋₆ alkyl, C₁₋₆ alkylidene, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —S(C₁₋₆ alkyl), —S(C₁₋₆ haloalkyl), —NRₐR_{b}, —NHCO(C₁₋₆ alkyl), —CO(C₁₋₆ alkyl), —CONH₂, —CONH(C₁₋₆ alkyl), —CON(C₁₋₆ alkyl)₂, —S(O)₂(C₁₋₆ alkyl), —S(O)₂NH(C₁₋₆ alkyl), and —S(O)₂N(C₁₋₆ alkyl)2;
R₄ is selected from C₁₋₆ alkyl, C₂₋₆ alkenyl, C₃₋₁₀ cycloalkyl, 4-12 membered heterocyclyl and 5-12 membered heteroaryl, each of which is optionally substituted with one or more R₁₃;
R₁₃ is selected from deuterium, halogen, —CN, —OH, —SH, oxo, —NH₂, C₁₋₆ alkyl, C₂₋₆ alkenyl, C₂₋₆ alkynyl, C₁₋₆ haloalkyl, C₃₋₈ cycloalkyl, 3-8 membered heterocyclyl, 6-10 membered aryl, 5-10 membered heteroaryl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —S(C₁₋₆ alkyl), —S(C₁₋₆ haloalkyl), —NHCO(C₁₋₆ alkyl), —NH(C₁₋₆ alkyl), —N(C₁₋₆ alkyl)₂, —CO(C₁₋₆ alkyl), —CONH₂, —CONH(C₁₋₆ alkyl), —CON(C₁₋₆ alkyl)₂, —S(O)₂(C₁₋₆ alkyl), —S(O)₂NH(C₁₋₆ alkyl), and —S(O)₂N(C₁₋₆ alkyl)₂; wherein the C₃₋₈ cycloalkyl, 3-8 membered heterocyclyl, 6-10 membered aryl and 5-10 membered heteroaryl are each optionally substituted with one or more R₁₄;
R₁₄ is independently selected from deuterium, halogen, —CN, —OH, —SH, oxo, C₁₋₆ alkyl, C₁₋₆ haloalkyl, C₃₋₈ cycloalkyl, 3-8 membered heterocyclyl, phenyl, 5-6 membered heteroaryl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —O(C₃₋₈ cycloalkyl), —(3-8 membered heterocyclyl), —S(C₁₋₆ alkyl), —S(C₁₋₆ haloalkyl), —S(C₃₋₈ cycloalkyl), —S(3-8 membered heterocyclyl), —NHCO(C₁₋₆ alkyl), —NRₐR_{b}, —C(O)NRₐR_{b}, —C(O)OR_{c}, —C(O)R_{d}, —S(O)₂R_{f}, and —S(O)₂NRₐR_{b}; wherein the C₁₋₆ alkyl is optionally substituted with one or more deuterium, and the C₃₋₈ cycloalkyl, 3-8 membered heterocyclyl, phenyl and 5-6 membered heteroaryl are each optionally substituted with one or more halogen;
R₅, R₆, R₇, R₈, R₉ and R₁₀ are each independently selected from hydrogen, deuterium, halogen, —CN, —OH, —SH, oxo, —NH₂, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O(C₁₋₆ alkyl), —O(C₁₋₆ haloalkyl), —NH(C₁₋₆alkyl), —N(C₁₋₆ alkyl)₂, —CO(C₁₋₆ alkyl), —CONH₂, —CONH(C₁₋₆ alkyl), —CON(C₁₋₆ alkyl)₂, C₃₋₈ cycloalkyl, and 3-8 membered heterocyclyl; or R₅ and R₆, R₇ and R₈, R₉ and R₁₀, R₅ and R₇, R₇ and R₉, or R₈ and R₉ together with the carbon atoms to which they are attached form C₃₋₆ cycloalkyl or 3-6 membered heterocyclyl;
or R₄ and R₅ together with the nitrogen atom and carbon atom to which they are attached form 5-12 membered heteroaryl, which is optionally substituted with one or more R₁₄;
or R₄ and R₅ together with the nitrogen atom and carbon atom to which they are attached form 4-10 membered heterocyclyl; wherein the 4-10 membered heterocyclyl is substituted with one or more groups independently selected from: C₃₋₈ cycloalkyl, 3-8 membered heterocyclyl, 6-10 membered aryl, and 5-10 membered heteroaryl; and the 4-10 membered heterocyclyl is further optionally substituted with one or more groups independently selected from: deuterium, halogen, —CN, —OH, —SH, oxo, —NH₂, C₁₋₆ alkyl, C₂₋₆ alkenyl, C₂₋₆ alkynyl, C₁₋₆ haloalkyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —S(C₁₋₆ alkyl), —S(C₁₋₆ haloalkyl), —NHCO(C₁₋₆ alkyl), —NH(C₁₋₆ alkyl), —N(C₁₋₆ alkyl)₂, —CO(C₁₋₆ alkyl), —CONH₂, —CONH(C₁₋₆ alkyl), —CON(C₁₋₆ alkyl)₂, —S(O)₂(C₁₋₆ alkyl), —S(O)₂NH(C₁₋₆ alkyl), and —S(O)₂N(C₁₋₆ alkyl)₂; wherein the C₃₋₈ cycloalkyl, 3-8 membered heterocyclyl, 6-10 membered aryl and 5-10 membered heteroaryl are each optionally substituted with one or more R₁4; and
Rₐ, R_{b}, R_{c}, R_{d} and R_{f} are each independently selected from hydrogen, C₁₋₆ alkyl, C₃₋₈ cycloalkyl, 3-8 membered heterocyclyl, phenyl, and 5-6 membered heteroaryl; wherein the C₁₋₆ alkyl, C₃₋₈ cycloalkyl, 3-8 membered heterocyclyl, phenyl and 5-6 membered heteroaryl are each optionally substituted with one or more groups independently selected from: halogen, —CN, —OH, —O(C₁₋₆ alkyl), C₃₋₈ cycloalkyl, 3-8 membered heterocyclyl, —NR_{g}Rₕ, and —NR_{g}C(O)Rᵢ; wherein R_{g}, Rₕ and Rᵢ are each independently selected from hydrogen, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, —(C₁₋₆ alkyl)-NH₂, —(C₁₋₆ alkyl)-NH(C₁₋₆ alkyl), —(C₁₋₆ alkyl)-N(C₁₋₆ alkyl)₂, C₃₋₈ cycloalkyl, 3-8 membered heterocyclyl, phenyl, and 5-6 membered heteroaryl.

3. The compound or the pharmaceutically acceptable salt thereof, or the solvate, the racemic mixture, the enantiomer, the diastereomer or the tautomer thereof according to claim 1 or 2, wherein X is O.

4. The compound or the pharmaceutically acceptable salt thereof, or the solvate, the racemic mixture, the enantiomer, the diastereomer or the tautomer thereof according to claim 1 or 2, wherein Y is N, and Z is N.

5. The compound or the pharmaceutically acceptable salt thereof, or the solvate, the racemic mixture, the enantiomer, the diastereomer or the tautomer thereof according to claim 1 or 2, wherein the compound is a compound of formula (I-1):

[symbol]

6. The compound or the pharmaceutically acceptable salt thereof, or the solvate, the racemic mixture, the enantiomer, the diastereomer or the tautomer thereof according to any one of claims 1-5, wherein p is 0 or 1; and preferably, p is 0.

7. The compound or the pharmaceutically acceptable salt thereof, or the solvate, the racemic mixture, the enantiomer, the diastereomer or the tautomer thereof according to any one of claims 1-6, wherein R₁ is —L₁—R₁₈; L₁ is absent, or L₁ is NR₁₁, wherein R₁₁ is independently selected from hydrogen, C₁₋₆ alkyl and C₁₋₆ haloalkyl; preferably, L₁ is absent, or L₁ is NH; and more preferably, L₁ is absent.

8. The compound or the pharmaceutically acceptable salt thereof, or the solvate, the racemic mixture, the enantiomer, the diastereomer or the tautomer thereof according to claim 7, wherein R₁₈ is selected from 6-10 membered aryl, 5-14 membered heteroaryl, C₃₋₈ cycloalkyl and 4-8 membered heterocyclyl, preferably selected from 6-10 membered aryl and 5-13 membered heteroaryl, more preferably 6-10 membered aryl and 8-13 membered heteroaryl, most preferably phenyl, naphthyl, 9-10 membered heteroaryl and 12-13 membered heteroaryl, each of which is optionally substituted with one or more groups independently selected from: halogen, —CN, —OH, —SH, —SF₅, oxo, C₁₋₆ alkyl, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —OC(O)NRₐR_{b}, —NRₐC(O)OR_{c}, —NRₐC(O)R_{d}, —NRₐR_{b}, and —C(O)NRₐR_{b}; wherein the C₁₋₆ alkyl is optionally substituted with one or more deuterium, and the C₂₋₆ alkenyl, C₂₋₆ alkynyl, C₃₋₈ cycloalkyl and 3-8 membered heterocyclyl are each optionally substituted with one or more halogen.

9. The compound or the pharmaceutically acceptable salt thereof, or the solvate, the racemic mixture, the enantiomer, the diastereomer or the tautomer thereof according to claim 7, wherein R₁₈ is selected from phenyl, naphthyl, indanyl, benzocyclobutenyl, pyridyl, pyrimidinyl, pyrazinyl, indazolyl, indolyl, benzothiazolyl, benzothienyl, benzimidazolyl, benzindazolyl, tetrahydrobenzindazolyl, quinolyl, quinolinonyl, tetrahydroquinolyl, isoquinolyl, pyrazoloquinolyl, pyrrolopyridyl, triazolopyridyl, tetrahydrocyclopentaindazolyl, dihydrocyclopentaindazolyl, dihydroindenothiazolyl, hexahydroindenoxazinyl, tetrahydrocyclopentaindolyl, tetrahydroindenoimidazolyl, cyclohexyl, and cyclohexenyl, each of which is optionally substituted with one or more groups independently selected from: halogen, —CN, —OH, —SH, —SF₅, oxo, C₁₋₆ alkyl, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —OC(O)NRₐR_{b}, —NRₐC(O)R_{d}, —NRₐR_{b}, and —C(O)NRₐR_{b}; wherein the C₁₋₆ alkyl is optionally substituted with one or more deuterium, and the C₂₋₆ alkenyl, C₂₋₆ alkynyl, C₃₋₈ cycloalkyl and 3-8 membered heterocyclyl are each optionally substituted with one or more halogen.

10. The compound or the pharmaceutically acceptable salt thereof, or the solvate, the racemic mixture, the enantiomer, the diastereomer or the tautomer thereof according to claim 7, wherein R₁₈ is selected from phenyl, naphthyl, indanyl, benzocyclobutenyl, pyridyl, pyrimidinyl, pyrazinyl, indazolyl, benzothiazolyl, quinolyl, isoquinolyl, cyclohexyl, and cyclohexenyl, each of which is optionally substituted with one or more groups independently selected from: halogen, —CN, —OH, —SH, oxo, C₁₋₆ alkyl, C₂₋₆ alkenyl, C₂₋₆ alkynyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —NRₐC(O)Rₐ, and —NRₐR_{b}; wherein the C₁₋₆ alkyl is optionally substituted with one or more deuterium, and the C₂₋₆ alkenyl, C₂₋₆ alkynyl, C₃₋₈ cycloalkyl and 3-8 membered heterocyclyl are each optionally substituted with one or more halogen.

11. The compound or the pharmaceutically acceptable salt thereof, or the solvate, the racemic mixture, the enantiomer, the diastereomer or the tautomer thereof according to any one of claims 8-10, wherein R₁₈ is selected from 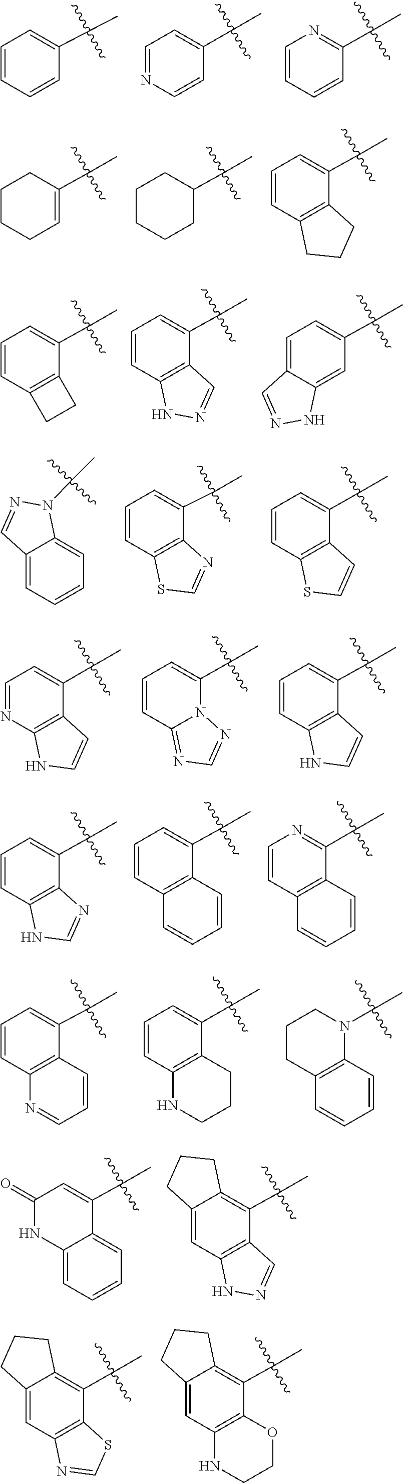 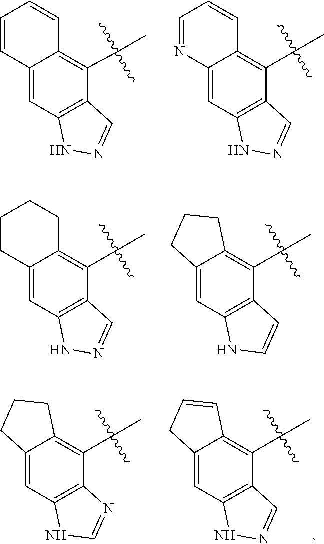 [SMILES: *c1c2ccccc2cc2[nH]ncc12 ; *c1c2cccnc2cc2[nH]ncc12 ; *c1c2c(cc3[nH]ncc13)CCCC2 ; *c1c2c(cc3[nH]ccc13)CCC2 ; *c1c2c(cc3[nH]cnc13)CCC2 ; *c1c2c(cc3[nH]ncc13)CC=C2]
each of which is optionally substituted with one or more groups independently selected from: halogen, —CN, —OH, C₁₋₆ alkyl, C₂₋₆ alkenyl, C₂₋₆ alkynyl, C₁₋₆ haloalkyl, C₃₋₈ cycloalkyl, —O(C₁₋₆ alkyl), —OC(O)NRₐR_{b}, —NRₐC(O)R_{d}, and —NRₐR_{b}; wherein Rₐ, R_{b} and 5 R_{d} are each independently selected from hydrogen and C₁₋₆ alkyl; wherein the C₁₋₆ alkyl in Rₐ, R_{b} and R_{d} is optionally substituted with one or more groups independently selected from: —OH, —NR_{g}Rₕ, —C(O)NR_{g}R_{b}, and —NHC(O)Rᵢ; wherein R_{g}, Rᵢ, and Rᵢ are each independently selected from hydrogen, C₁₋₆ alkyl, and —(C₁₋₆ alkyl)-NH₂; or Rₐ and R_{b} together with the nitrogen atom to which they are attached form 3-6 membered heterocyclyl; preferably, R₁₈ is selected from 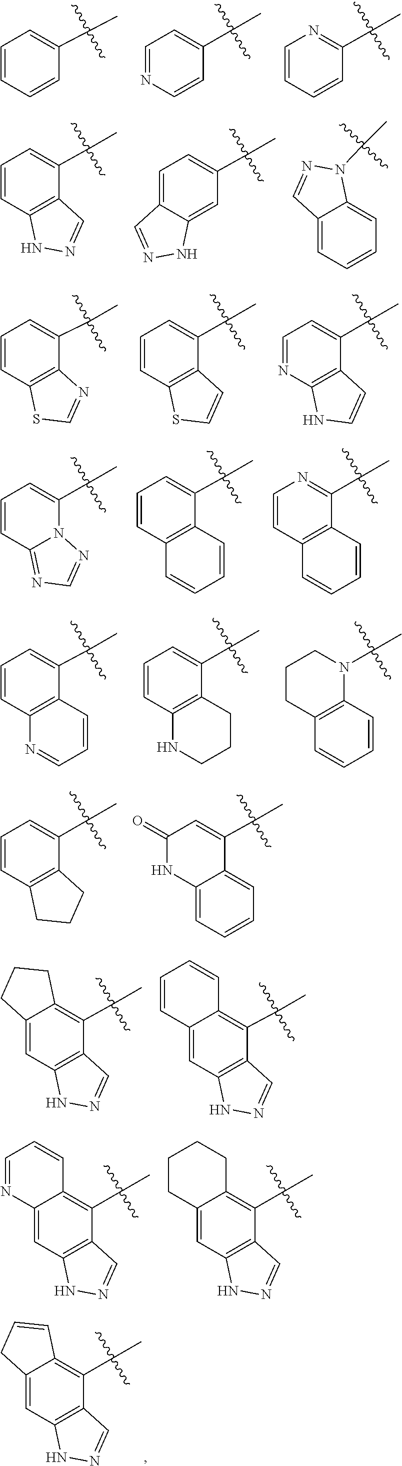
each of which is optionally substituted with one or more groups independently selected from: halogen, —OH, C₁₋₆ alkyl, C₂₋₆ alkynyl, C₁₋₆ haloalkyl, C₃₋₈ cycloalkyl, —O(C₁₋₆ alkyl), —OC(O)NRₐR_{b}, —NRₐC(O)Rₐ, and —NRₐR_{b}; wherein Rₐ, R_{b} and R_{d} are each independently selected from hydrogen and C₁₋₆ alkyl; wherein the C₁₋₆ alkyl in Rₐ, R_{b} and R_{d} is optionally substituted with one or more groups independently selected from: —OH, —NR_{g}Rₕ, —C(O)NR_{g}Rₕ, and —NHC(O)Rᵢ; wherein R_{g}, Rₕ and R₁ are each independently selected from hydrogen, C₁₋₆ alkyl, and —(C₁₋₆ alkyl)-NH₂; or Rₐ and Re together with the nitrogen atom to which they are attached form 3-6 membered heterocyclyl;
more preferably, R₁₈ is selected from 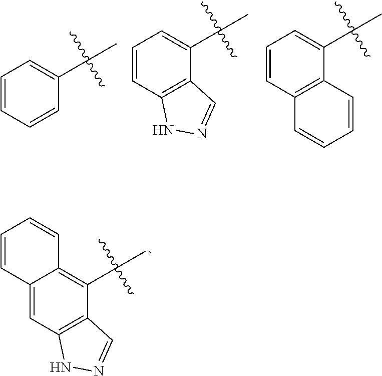 [SMILES: *c1ccccc1 ; *c1cccc2[nH]ncc12 ; *c1cccc2ccccc12 ; *c1c2ccccc2cc2[nH]ncc12]
each of which is optionally substituted with one or more groups independently selected from: halogen, —OH, C₁₋₆ alkyl, C₂₋₆ alkynyl, C₁₋₆ haloalkyl, C₃₋₈ cycloalkyl, —O(C₁₋₆ alkyl), —NRₐC(O)R_{d}, and —NRₐR_{b}; wherein Rₐ, R_{b} and R_{d} are each independently selected from hydrogen and C₁₋₆ alkyl; wherein the C₁₋₆ alkyl in Rₐ, R_{b} and R_{d} is optionally substituted with one or more groups independently selected from: —OH, —NR_{g}Rₕ and —NHC(O)Rᵢ; wherein R_{g}, Rₕ and Rᵢ are each independently selected from hydrogen, C₁₋₆ alkyl, and —(C₁₋₆ alkyl)-NH₂;
further preferably, R₁₈ is 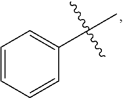 [SMILES: *c1ccccc1]
which is optionally substituted with one or more groups independently selected from: halogen, —OH, C₁₋₆ alkyl, C₂₋₆ alkynyl, C₁₋₆ haloalkyl, C₃₋₈ cycloalkyl, and —NH₂; or R₁₈ is 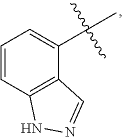 [SMILES: *c1cccc2[nH]ncc12]
which is optionally substituted with one or more groups independently selected from: halogen, C₁₋₆ alkyl, C₂₋₆ alkynyl, C₁₋₆ haloalkyl, C₃₋₈ cycloalkyl, and —NH₂; or R₁₈ is 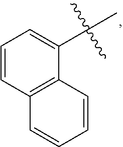 [SMILES: *c1cccc2ccccc12]
which is optionally substituted with one or more groups independently selected from: halogen, —OH, C₁₋₆ alkyl, and C₂₋₆ alkynyl; or R₁₈ is 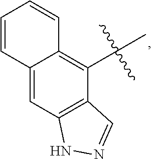 [SMILES: *c1c2ccccc2cc2[nH]ncc12]
which is optionally substituted with one or more groups independently selected from: halogen, C₁₋₆ alkyl, and C₃₋₈ cycloalkyl.

12. The compound or the pharmaceutically acceptable salt thereof, or the solvate, the racemic mixture, the enantiomer, the diastereomer or the tautomer thereof according to any one of claims 1-11, wherein R₂ is selected from hydrogen, halogen, —CN, —OH, —SH, C₁₋₆ alkyl, C₂₋₆ alkenyl, C₂₋₆ alkynyl, C₁₋₆ haloalkyl, C₃₋₈ cycloalkyl, —O(C₁₋₆ alkyl), and —NRₐR_{b}; wherein the C₁₋₆ alkyl is optionally substituted with one or more deuterium, and the C₂₋₆ alkenyl, C₂₋₆ alkynyl and C₃₋₈ cycloalkyl are each optionally substituted with one or more halogen; preferably, R₂ is selected from hydrogen, halogen, —CN, —OH, C₁₋₆ alkyl, C₂₋₆ alkynyl, C₁₋₆, haloalkyl, C₃₋₈ cycloalkyl, —O(C₁₋₆ alkyl), and —NH₂; and more preferably, R₂ is halogen, for example, F.

13. The compound or the pharmaceutically acceptable salt thereof, or the solvate, the racemic mixture, the enantiomer, the diastereomer or the tautomer thereof according to any one of claims 1-12, wherein R₃ is selected from hydrogen, —OH, —O(C₁₋₆ alkyl), —O(C₁₋₆ haloalkyl), —S(C₁₋₆ alkyl), —S(C₁₋₆ haloalkyl), C₃₋₈ cycloalkyl, 4-10 membered heterocyclyl, and —O—L₂—R₁₂; wherein the C₃₋₈ cycloalkyl and 4-10 membered heterocyclyl are each optionally substituted with one or more groups independently selected from: deuterium, halogen, —CN, —OH, oxo, C₁₋₆ alkyl, C₁₋₆ alkylidene, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, and —O(C₁₋₆ alkyl), wherein the C₁₋₆ alkylidene is optionally substituted with one or more halogen; preferably, R₃ is selected from hydrogen, —O(C₁₋₆ alkyl), —S(C₁₋₆ alkyl), 4-10 membered heterocyclyl, and —O—L₂—R₁₂; wherein the 4-10 membered heterocyclyl is optionally substituted with one or more groups independently selected from: deuterium, halogen, and —(C₁₋₆ alkyl)-OH; and more preferably, R₃ is —O-L₂—R₁₂.

14. The compound or the pharmaceutically acceptable salt thereof, or the solvate, the racemic mixture, the enantiomer, the diastereomer or the tautomer thereof according to claim 13, wherein L₂ is C₁₋₆ alkylene or C₃₋₈ cycloalkylene, each of which is optionally substituted with one or more deuterium or halogen; preferably, L₂ is C₁₋₆ alkylene, which is optionally substituted with one or more deuterium; and more preferably, L₂ is CH₂, which is optionally substituted with one or more deuterium.

15. The compound or the pharmaceutically acceptable salt thereof, or the solvate, the racemic mixture, the enantiomer, the diastereomer or the tautomer thereof according to either of claims 13-14, wherein R₁₂ is C₃₋₈ cycloalkyl or 4-10 membered heterocyclyl, preferably 4-10 membered heterocyclyl, more preferably 4-8 membered heterocyclyl, each of which is optionally substituted with one or more groups independently selected from: deuterium, halogen, —CN, —OH, oxo, C₁₋₆ alkyl, C₁₋₆ alkylidene, C₁₋₆ haloalkyl, C₃₋₈ cycloalkyl, 3-8 membered heterocyclyl, —O(C₁₋₆ alkyl), —O(C₁₋₆ haloalkyl), —NRₐC(O)Rₐ, —NRₐR_{b}, —C(O)NRₐR_{b}, —C(O)OR_{c}, and —C(O)R_{d}; wherein the C₁₋₆ alkyl, C₁₋₆ alkylidene, C₃₋₈ cycloalkyl and 3-8 membered heterocyclyl are each optionally substituted with one or more R₁₇.

16. The compound or the pharmaceutically acceptable salt thereof, or the solvate, the racemic mixture, the enantiomer, the diastereomer or the tautomer thereof according to claim 15, wherein R₁₂ is selected from cyclopropyl, cyclobutyl, azetidinyl, pyrrolidinyl, morpholinyl, piperidinyl, piperazinyl, tetrahydropyranyl, hexahydro-1H-pyrrolizinyl, tetrahydro-1′H,3′H-spiro[cyclopropane-1,2′-pyrrolizinyl]and octahydrocyclopropa[a]pyrrolizinyl, each of which is optionally substituted with one or 5 more groups independently selected from: deuterium, halogen, —CN, —OH, oxo, C₁₋₆ alkyl, C₁₋₆ alkylidene, C₁₋₆ haloalkyl, C₃₋₈ cycloalkyl, 3-8 membered heterocyclyl, —O(C₁₋₆ alkyl), —O(C₁₋₆ haloalkyl), —NRₐC(O)R_{d}, —NRₐR_{b}, —C(O)NRₐR_{b}, —C(O)ORₑ, and —C(O)Rₐ; wherein the C₁₋₆ alkyl, C₁₋₆ alkylidene, C₃₋₈ cycloalkyl and 3-8 membered heterocyclyl are each optionally substituted with one or more R₁₇.

17. The compound or the pharmaceutically acceptable salt thereof, or the solvate, the racemic mixture, the enantiomer, the diastereomer or the tautomer thereof according to claim 16, wherein R₁₂ is selected from 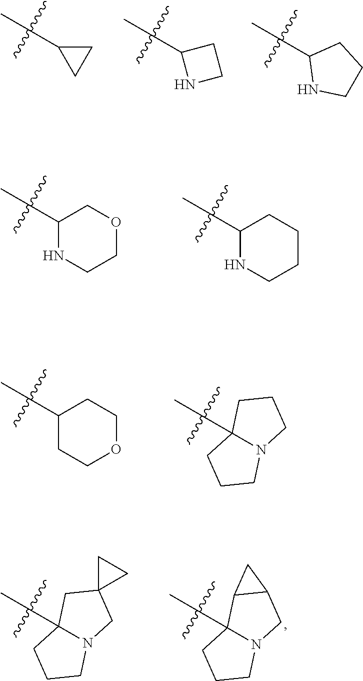
each of which is optionally substituted with one or more groups independently selected from: deuterium, halogen, —CN, —OH, oxo, C₁₋₆ alkyl, C₁₋₆ alkylidene, C₁₋₆ haloalkyl, C₃₋₈ cycloalkyl, 3-8 membered heterocyclyl, and —O(C₁₋₆ alkyl); wherein the C₁₋₆ alkyl, C₁₋₆ alkylidene, C₃₋₈ cycloalkyl and 3-8 membered heterocyclyl are each optionally substituted with one or more R₁₇; preferably, R₁₂ is selected from 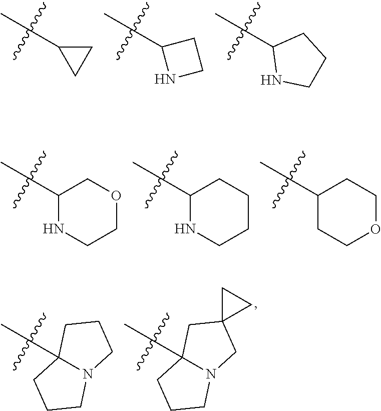
each of which is optionally substituted with one or more groups independently selected from: deuterium, halogen, —CN, oxo, C₁₋₆ alkyl, C₁₋₆ alkylidene, C₁₋₆ haloalkyl, and 3-8 membered heterocyclyl; wherein the C₁₋₆ alkyl, C₁₋₆ alkylidene and 3-8 membered heterocyclyl are each optionally substituted with one or more R₁₇;
more preferably, R₁₂ is selected from 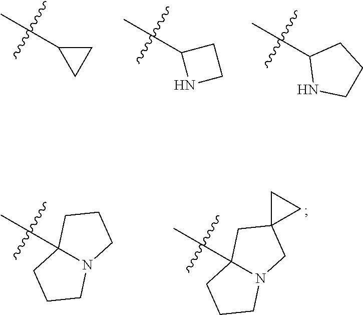
each of which is optionally substituted with one or more groups independently selected from: deuterium, halogen, —CN, oxo, C₁₋₆ alkyl, C₁₋₆ alkylidene, C₁₋₆ haloalkyl, and 3-8 membered heterocyclyl; wherein the C₁₋₆ alkyl, C₁₋₆ alkylidene and 3-8 membered heterocyclyl are each optionally substituted with one or more R₇;
further preferably, R₁₂ is 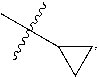 [SMILES: *C1CC1]
which is optionally substituted with one or more groups independently selected from: —CN and C₁₋₆ alkyl; wherein the C₁₋₆ alkyl is optionally substituted with one or more R₁₇; or R₁₂ is 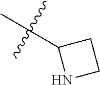 [SMILES: *C1CCN1]
which is optionally substituted with one or more groups independently selected from: C₁₋₆ haloalkyl; or R₁₂ is 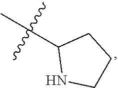 [SMILES: *C1CCCN1 ; ","]
which is optionally substituted with one or more groups independently selected from: deuterium, halogen, C₁₋₆ alkyl, C₁₋₆ haloalkyl, and 3-8 membered heterocyclyl; wherein the C₁₋₆ alkyl and 3-8 membered heterocyclyl are each optionally substituted with one or more R₁₇; or R₁₂ is 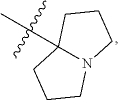 [SMILES: *C12CCCN1CCC2]
which is optionally substituted with one or more groups independently selected from: deuterium, halogen, and C₁₋₆ alkylidene; wherein the C₁₋₆ alkylidene is optionally substituted with one or more R₁₇.

18. The compound or the pharmaceutically acceptable salt thereof, or the solvate, the racemic mixture, the enantiomer, the diastereomer or the tautomer thereof according to any one of claims 15-17, wherein R₁₇ is selected from deuterium, halogen, —CN, —OH, oxo, 3-8 membered heterocyclyl, —O(C₁₋₆ alkyl), —O(C₁₋₆ haloalkyl), —NRₐR_{b}, and —NHCO(C₁₋₆ alkyl), wherein the 3-8 membered heterocyclyl is optionally substituted with one or more groups independently selected from: halogen, —CN, —OH, oxo, C₁₋₆ alkyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —CO(C₁₋₆ alkyl), and —S(O)₂(C₁₋₆ alkyl); preferably, R₁₇ is selected from deuterium, halogen, —OH, 3-8 membered heterocyclyl, and —NRₐR_{b}, wherein the 3-8 membered heterocyclyl is optionally substituted with one or more groups independently selected from: halogen, C₁₋₆ alkyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —CO(C₁₋₆ alkyl), and —S(O)₂(C₁₋₆ alkyl); more preferably, R₁₇ is selected from deuterium, halogen, —OH, 3-8 membered heterocyclyl, —N(C₁₋₆ alkyl)₂, and —N(C₁₋₆ alkyl)(C₃₋₈ cycloalkyl), wherein the C₁₋₆ alkyl is optionally substituted with one or more —OH, and the 3-8 membered heterocyclyl is optionally substituted with one or more groups independently selected from: C₃₋₈ cycloalkyl, 3-8 membered heterocyclyl, and —(C₁₋₆ alkyl)-OH; and further preferably, R₁₇ is selected from deuterium, and halogen, for example, F.

19. The compound or the pharmaceutically acceptable salt thereof, or the solvate, the racemic mixture, the enantiomer, the diastereomer or the tautomer thereof according to any one of claims 1-18, wherein R₄ is selected from C₁₋₆ alkyl, C₃₋₈ cycloalkyl, 4-8 membered heterocyclyl, and 5-10 membered heteroaryl, each of which is optionally substituted with one or more R₁₃; preferably, R₄ is selected from C₁₋₆ alkyl, C₃₋₈ cycloalkyl, and 4-8 membered heterocyclyl, each of which is optionally substituted with one or more R₁₃; more preferably, R₄ is selected from C₁₋₆ alkyl, and C₃₋₈ cycloalkyl, each 5 of which is optionally substituted with one or more R₁₃; further preferably, R₄ is C₁₋₆ alkyl, which is optionally substituted with one or more R₁₃; and most preferably, R₄ is C₁₋₆ alkyl, which is substituted with one or more R₁₃.

20. The compound or the pharmaceutically acceptable salt thereof, or the solvate, the racemic mixture, the enantiomer, the diastereomer or the tautomer thereof according to claim 19, wherein R₁₃ is selected from deuterium, halogen, —OH, oxo, —NH₂, C₃₋₈ cycloalkyl, 3-8 membered heterocyclyl, 6-10 membered aryl, 5-10 membered heteroaryl, —O(C₁₋₆ alkyl), —NHCO(C₁₋₆ alkyl), —NH(C₁₋₆ alkyl), and —N(C₁₋₆ alkyl)₂; wherein the C₃₋₈ cycloalkyl, 3-8 membered heterocyclyl, 6-10 membered aryl and 5-10 membered heteroaryl are each optionally substituted with one or more R₁₄; preferably, R₁₃ is selected from deuterium, oxo, —NH₂, and 5-10 membered heteroaryl; wherein the 5-10 membered heteroaryl is optionally substituted with one or more R₁₄; more preferably, R₁₃ is selected from deuterium, and 5-10 membered heteroaryl; wherein the 5-10 membered heteroaryl is optionally substituted with one or more R₁₄; and further preferably, R₁₃ is 5-6 membered heteroaryl, which is optionally substituted with one or more R₁₄.

21. The compound or the pharmaceutically acceptable salt thereof, or the solvate, the racemic mixture, the enantiomer, the diastereomer or the tautomer thereof according to claim 20, wherein R₁₃ is selected from phenyl, pyrazolyl, pyridyl, pyridazinyl, pyrimidinyl, pyrazinyl, and imidazopyridyl, each of which is optionally substituted with one or more R₁₄.

22. The compound or the pharmaceutically acceptable salt thereof, or the solvate, the racemic mixture, the enantiomer, the diastereomer or the tautomer thereof according to claim 21, wherein R₁₃ is selected from 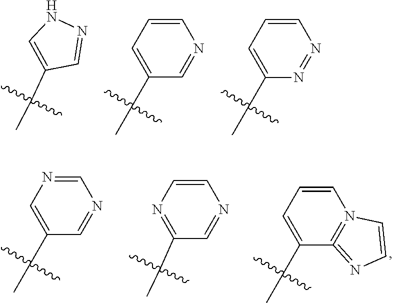
each of which is optionally substituted with one or more R₁₄;
preferably, R₁₃ is selected from 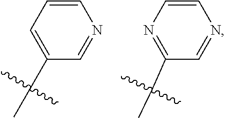 [SMILES: *c1cccnc1 ; *Cc1cnccn1]
each of which is optionally substituted with one or more R₁₄;
more preferably, R₁₃ is 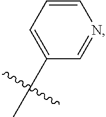 [SMILES: *c1cccnc1]
which is optionally substituted with one or more R₁₄; or R₁₃ is 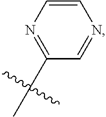 [SMILES: *c1cnccn1]
which is optionally substituted with one or more R₁₄.

23. The compound or the pharmaceutically acceptable salt thereof, or the solvate, the racemic mixture, the enantiomer, the diastereomer or the tautomer thereof according to any one of claims 20-22, wherein R₁₄ is independently selected from deuterium, halogen, —CN, —OH, oxo, C₁₋₆ alkyl, C₁₋₆ haloalkyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —O(C₁₋₆ haloalkyl), —NRₐC(O)R_{d}, —NRₐR_{b}, and —C(O)NRₐR_{b}; wherein the C₁₋₆ alkyl is optionally substituted with one or more deuterium; preferably, R₁₄ is independently selected from halogen and —NRₐR_{b}; and more preferably, R₁₄ is —NH₂.

24. The compound or the pharmaceutically acceptable salt thereof, or the solvate, the racemic mixture, the enantiomer, the diastereomer or the tautomer thereof according to any one of claims 1-23, wherein R₅, R₆, R₇, R₈, R₉ and R₁₀ are each independently selected from hydrogen, deuterium, C₁₋₆ alkyl, C₁₋₆ haloalkyl, and C₃₋₈ cycloalkyl; or R₅ and R₆, R₇ and R₈, R₉ and R₁₀, R₅ and R₇, R₇ and R₉, or R₅ and R₉ together with the carbon atoms to which they are attached form C₃₋₆ cycloalkyl or 3-6 membered heterocyclyl; preferably, R₅, R₆, R₇, R₈, R₉ and R₁₀ are each independently selected from hydrogen, deuterium and C₁₋₃ alkyl; or R₅ and R₆ together with the carbon atom to which they are attached form cyclopropyl; or R₅ and R₇ together with the carbon atoms to which they are attached form cyclopropyl, cyclopentyl, tetrahydrofuranyl, or tetrahydropyranyl; more preferably, R₅, R₆, R₇, R₈, R₉ and R₁₀ are all hydrogen; or R₅ is C₁₋₃ alkyl, and R₆, R₇, R₈, R₉ and R₁₀ are each independently selected from hydrogen and deuterium; or R₅ and R₇ together with the carbon atoms to which they are attached form cyclopentyl, tetrahydrofuranyl, or tetrahydropyranyl.

25. The compound or the pharmaceutically acceptable salt thereof, or the solvate, the racemic mixture, the enantiomer, the diastereomer or the tautomer thereof according to claim 1 or 2, wherein the compound is a compound of formula (I-2): 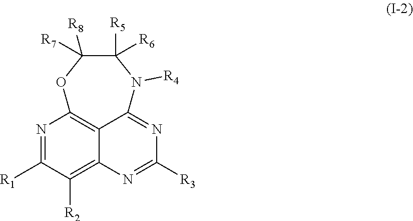
wherein:
R₁ is —L₁—R₁₈;
L₁ is absent;
R₁₈ is selected from 6-10 membered aryl and 5-14 membered heteroaryl, such as 5-10 membered heteroaryl, each of which is optionally substituted with one or more groups independently selected from: halogen, —CN, —OH, C₁₋₆ alkyl, C₂₋₆ alkenyl, C₂₋₆ alkynyl, C₁₋₆ haloalkyl, C₃₋₈ cycloalkyl, —O(C₁₋₆ alkyl), —OC(O)NRₐR_{b}, —NRₐC(O)Rₐ, and —NRₐR_{b}; preferably, R₁₈ is selected from 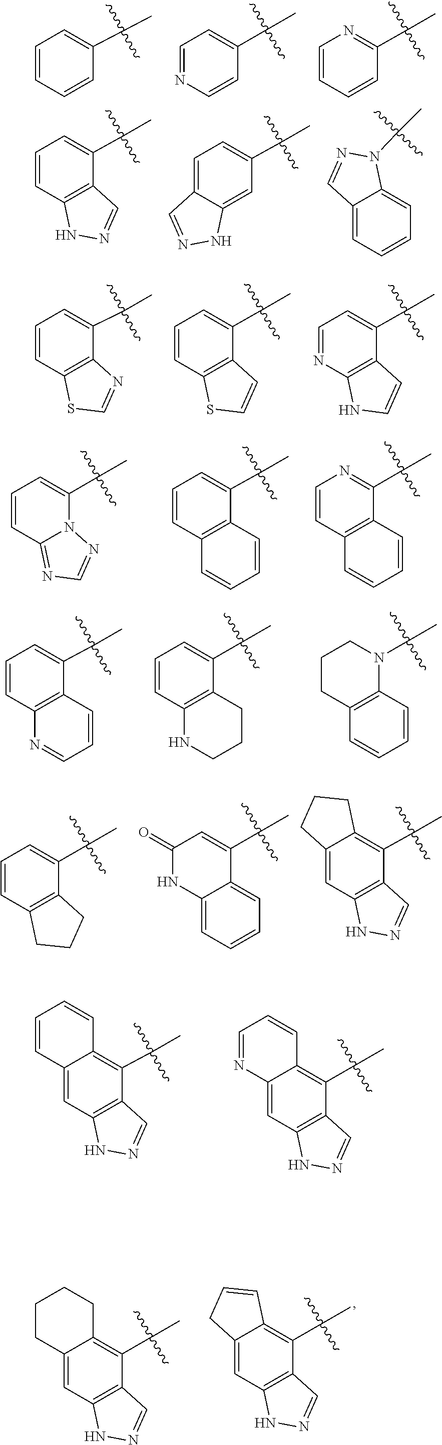
each of which is optionally substituted with one or more groups independently selected from: halogen, —OH, C₁₋₆ alkyl, C₂₋₆ alkynyl, C₁₋₆ haloalkyl, C₃₋₈ cycloalkyl, —O(C₁₋₆ alkyl), —OC(O)NRₐR_{b}NRₐC(O)R₄, and —NRₐR_{b}; more preferably, R₁₈ is selected from 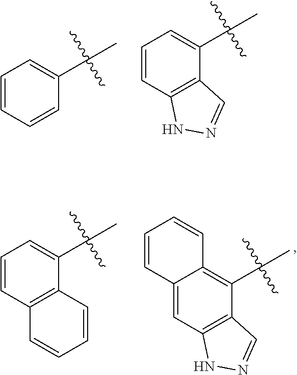 [SMILES: *c1ccccc1 ; *c1cccc2[nH]ncc12 ; *c1cccc2ccccc12 ; *c1c2ccccc2cc2[nH]ncc12]
each of which is optionally substituted with one or more groups independently selected from: halogen, —OH, C₁₋₆ alkyl, C₂₋₆ alkynyl, C₁₋₆ haloalkyl, C₃₋₈ cycloalkyl, —O(C₁₋₆ alkyl), —NRₐC(O)Rₐ, and —NRₐR_{b}; further preferably, R₁₈ is 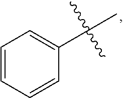 [SMILES: *c1ccccc1]
which is optionally substituted with one or more groups independently selected from: halogen, —OH, C₁₋₆ alkyl, C₂₋₆ alkynyl, C₁₋₆ haloalkyl, Cu cycloalkyl, and —NH₂; or R₁₈ is 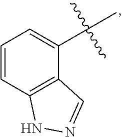 [SMILES: *c1cccc2[nH]ncc12]
which is optionally substituted with one or more groups independently selected from: halogen, C₁₋₆ alkyl, C₂₋₆ alkynyl, C₁₋₆ haloalkyl, C₃₋₈ cycloalkyl, and —NH₂; or R₁₈ is 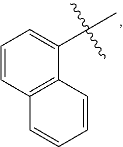 [SMILES: *c1cccc2ccccc12]
which is optionally substituted with one or more groups independently selected from: halogen, —OH, C₁₋₆ alkyl, and C₂₋₆ alkynyl; or R₁₈ is 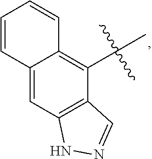 [SMILES: *c1c2ccccc2cc2[nH]ncc12]
which is optionally substituted with one or more groups independently selected from: halogen, C₁₋₆ alkyl, and C₃₋₈ cycloalkyl;
R₂ is selected from hydrogen, halogen, —CN, —OH, C₁₋₆ alkyl, C₂₋₆ alkynyl, C₁₋₆ haloalkyl, C₃₋₈ cycloalkyl, —O(C₁₋₆ alkyl), and —NH₂; and preferably, R₂ is halogen;
R₃ is —O—L₂—R₁₂;
L₂ is C₁₋₆ alkylene or C₃₋₈ cycloalkylene, each of which is optionally substituted with one or more deuterium or halogen; preferably, L₂ is C₁₋₆ alkylene, which is optionally substituted with one or more deuterium; and more preferably, L₂ is CH₂, which is optionally substituted with one or more deuterium;
R₁₂ is C₃₋₈ cycloalkyl or 4-10 membered heterocyclyl, each of which is optionally substituted with one or more groups independently selected from: deuterium, halogen, —CN, oxo, C₁₋₆ alkyl, C₁₋₆ alkylidene, C₁₋₆ haloalkyl, and 3-8 membered heterocyclyl;
wherein the C₁₋₆ alkyl, C₁₋₆ alkylidene and 3-8 membered heterocyclyl are each optionally substituted with one or more R₁₇; preferably, R₁₂ is selected from 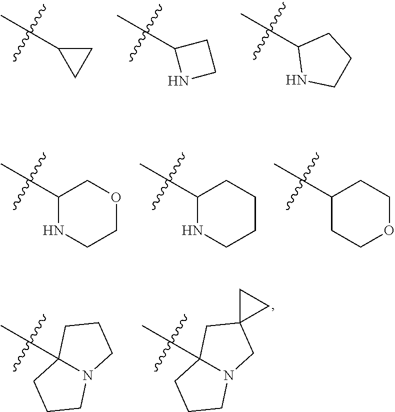
each of which is optionally substituted with one or more groups independently selected from: deuterium, halogen, —CN, oxo, C₁₋₆ alkyl, C₁₋₆ alkylidene, C₁₋₆ haloalkyl, and 3-8 membered heterocyclyl; wherein the C₁₋₆ alkyl, C₁₋₆ alkylidene and 3-8 membered heterocyclyl are each optionally substituted with one or more R₁₇; more preferably, R₁₂ is selected from 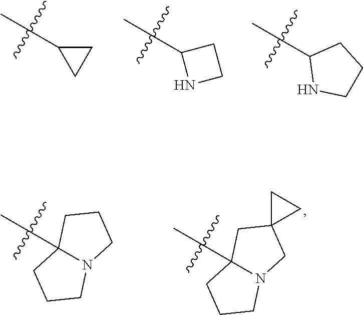
each of which is optionally substituted with one or more groups independently selected from: deuterium, halogen, —CN, oxo, C₁₋₆ alkyl, C₁₋₆ alkylidene, C₁₋₆ haloalkyl, and 3-8 membered heterocyclyl; wherein the C₁₋₆ alkyl, C₁₋₆ alkylidene and 3-8 membered heterocyclyl are each optionally substituted with one or more R₁₇; further preferably, R₁₂ is 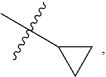 [SMILES: *C1CC1]
which is optionally substituted with one or more groups independently selected from: —CN and C₁₋₆ alkyl; wherein the C₁₋₆ alkyl is optionally substituted with one or more R₁₇; or R₁₂ is 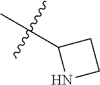 [SMILES: *C1CCN1]
which is optionally substituted with one or more groups independently selected from: C₁₋₆ haloalkyl; or R₁₂ is 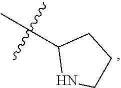 [SMILES: *C1CCCN1]
which is optionally substituted with one or more groups independently selected from: deuterium, halogen, C₁₋₆ alkyl, C₁₋₆ haloalkyl, and 3-8 membered heterocyclyl; wherein the C₁₋₆ alkyl and 3-8 membered heterocyclyl are each optionally substituted with one or more R₁₇; or R₁₂ is 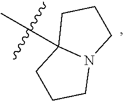 [SMILES: *C12CCCN1CCC2]
which is optionally substituted with one or more groups independently selected from: deuterium, halogen, and C₁₋₆ alkylidene; wherein the C₁₋₆ alkylidene is optionally substituted with one or more R₁₇;
R₁₇ is selected from deuterium, halogen, —OH, 3-8 membered heterocyclyl, and —NRₐR_{b}, wherein the 3-8 membered heterocyclyl is optionally substituted with one or more groups independently selected from: halogen, C₁₋₆ alkyl, C₁₋₆ haloalkyl, C₃₋₈ cycloalkyl, 3-8 membered heterocyclyl, —(C₁₋₆ alkyl)-OH, —(C₁₋₆ alkyl)-O—(C₁₋₆ alkyl), —(C₁₋₆ alkyl)-CN, —O(C₁₋₆ alkyl), —CO(C₁₋₆ alkyl), and —S(O)₂(C₁-(alkyl); preferably, R₁₇ is selected from deuterium, halogen, —OH, 3-8 membered heterocyclyl, —N(C₁₋₆ alkyl)₂, and —N(C₁₋₆ alkyl)(C₃₋₈ cycloalkyl), wherein the C₁₋₆ alkyl is optionally substituted with one or more —OH, and the 3-8 membered heterocyclyl is optionally substituted with one or more groups independently selected from: C₃₋₈ cycloalkyl, 3-8 membered heterocyclyl, and —(C₁₋₆ alkyl)-OH; and more preferably, R₁₇ is selected from deuterium, and halogen, for example, F;
R₄ is selected from C₁₋₆ alkyl, C₃₋₈ cycloalkyl, and 4-8 membered heterocyclyl, each of which is optionally substituted with one or more R₁₃; preferably, R₄ is selected from C₁₋₆ alkyl, and C₃₋₈ cycloalkyl, each of which is optionally substituted with one or more R₁₃; more preferably, R₄ is C₁₋₆ alkyl, which is optionally substituted with one or more R₁₃; and further preferably, R₄ is C₁₋₆ alkyl, which is substituted with one or more R₁₃;
R₁₃ is selected from deuterium and 5-10 membered heteroaryl; wherein the 5-10 membered heteroaryl is optionally substituted with one or more R₁4; preferably, R₁₃ is selected from 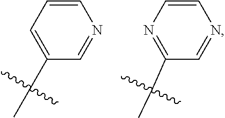 [SMILES: *c1cccnc1 ; *Cc1cnccn1]
each of which is optionally substituted with one or more R₁₄; more preferably, R₁₃ is 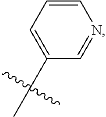 [SMILES: *c1cccnc1]
which is optionally substituted with one or more R₁₄; or R₁₃ is 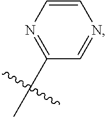 [SMILES: *c1cnccn1]
which is optionally substituted with one or more R₁₄;
R₁₄ is selected from halogen and —NRₐR_{b}; and preferably, R₁₄ is —NH₂;
R₅, R₆, R₇ and R₈ are each independently selected from hydrogen, deuterium, C₁₋₆ alkyl, C₁₋₆ haloalkyl, and C₃₋₈ cycloalkyl; or R₅ and R₆, R₇ and R₈, or R₅ and R₇ together with the carbon atoms to which they are attached form C₃₋₆ cycloalkyl or 3-6 membered heterocyclyl; preferably, R₅, R₆, R₇ and R₈ are each independently selected from hydrogen, deuterium and C₁₋₃ alkyl; or R₅ and R₆ together with the carbon atom to which they are attached form cyclopropyl; or R₅ and R₇ together with the carbon atoms to which they are attached form cyclopropyl, cyclopentyl, tetrahydrofuranyl, or tetrahydropyranyl; more preferably, R₅, R₆, R₇ and R₈ are all hydrogen; or R₅ is C₁₋₃ alkyl, and R₆, R₇ and R₈ are each independently selected from hydrogen and deuterium; or R₅ and R₇ together with the carbon atoms to which they are attached form cyclopentyl, tetrahydrofuranyl, or tetrahydropyranyl; and
Rₐ, R_{b} and R_{d} are each independently selected from hydrogen, C₁₋₆ alkyl, and C₃₋₈ cycloalkyl; wherein the C₁₋₆ alkyl and C₃₋₈ cycloalkyl are each optionally substituted with one or more groups independently selected from: —OH, —NR_{g}Rₕ, —C(O)NR_{g}Rₕ, and —NHC(O)Rᵢ; wherein R_{g}, Rₕ and Rᵢ are each independently selected from hydrogen, C₁₋₆ alkyl, and —(C₁₋₆ alkyl)-NH₂.

26. The compound or the pharmaceutically acceptable salt thereof, or the solvate, the racemic mixture, the enantiomer, the diastereomer or the tautomer thereof according to claim 25, wherein R₄ is C₁₋₆ alkyl substituted with R₁3;
R₁₃ is selected from 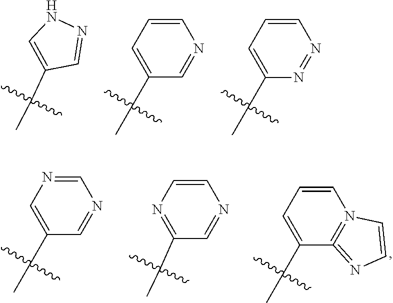
each of which is optionally substituted with one or more R₁₄;
preferably, R₁₃ is selected from 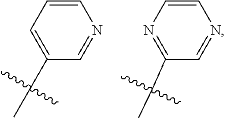 [SMILES: *c1cccnc1 ; *Cc1cnccn1]
each of which is optionally substituted with one or more R₁₄;
more preferably, R₁₃ is 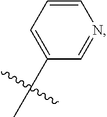 [SMILES: *c1cccnc1]
which is optionally substituted with one or more R₁₄; or R₁₃ is 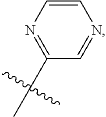 [SMILES: *c1cnccn1]
which is optionally substituted with one or more R₁₄; and
R₁₄ is independently selected from halogen and —NH₂.

27. A compound or the pharmaceutically acceptable salt thereof, or the solvate, the racemic mixture, the enantiomer, the diastereomer or the tautomer thereof, which is selected from:

| No. | Structural formula |
|---|---|
| 1 | 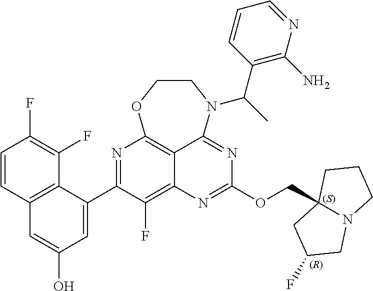 |
| 2 | 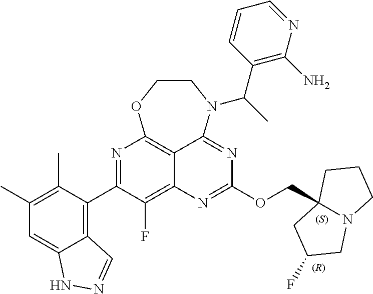 |
| 3 | 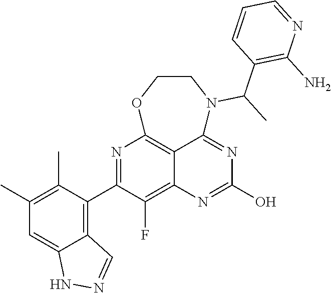 [SMILES: Cc1cc2[nH]ncc2c(-c2nc3c4c(nc(O)nc4c2F)N(C(C)c2cccnc2N)CCO3)c1C] |
| 4 | 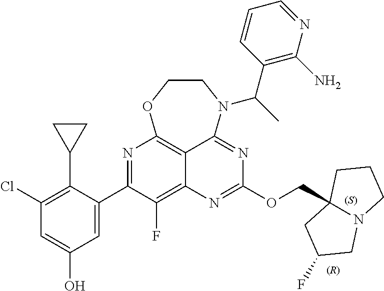 |
| 5 | 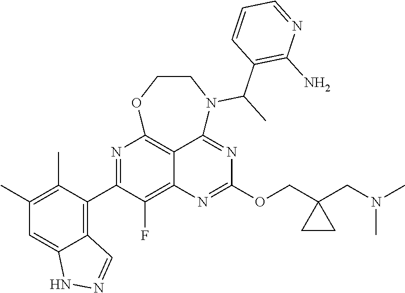 |
| 6 | 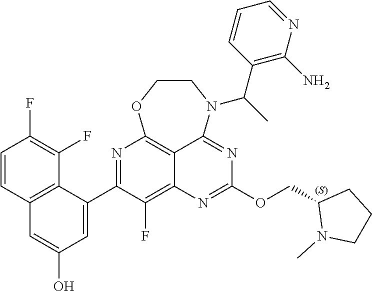 |
| 7 | 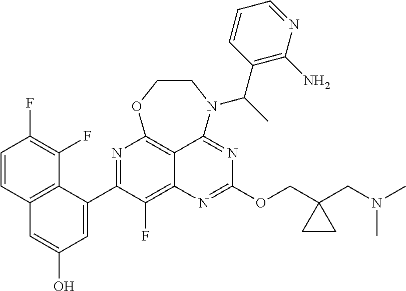 [SMILES: CC(c1cccnc1N)N1CCOc2nc(-c3cc(O)cc4ccc(F)c(F)c34)c(F)c3nc(OCC4(CN(C)C)CC4)nc1c23] |
| 8 | 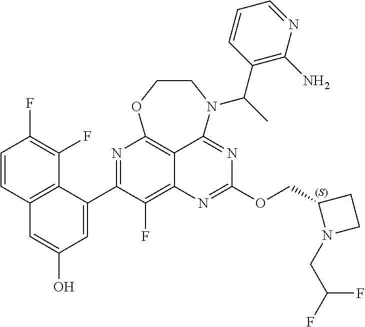 |
| 9 | 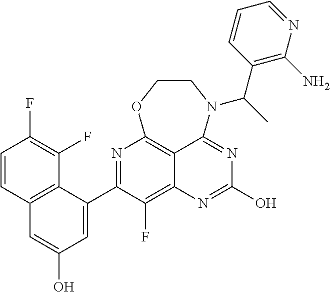 [SMILES: CC(c1cccnc1N)N1CCOc2nc(-c3cc(O)cc4ccc(F)c(F)c34)c(F)c3nc(O)nc1c23] |
| 10 | 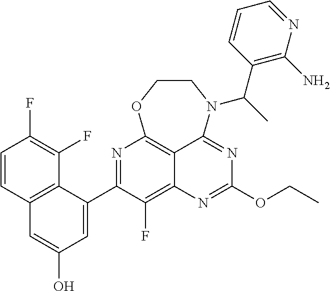 [SMILES: CCOc1nc2c3c(nc(-c4cc(O)cc5ccc(F)c(F)c45)c(F)c3n1)OCCN2C(C)c1cccnc1N] |
| 11 | 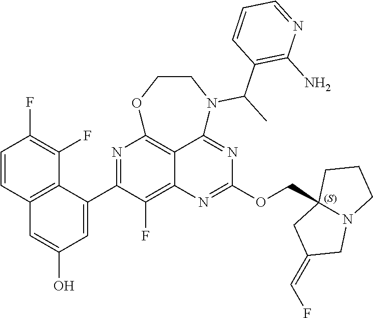 |
| 12 | 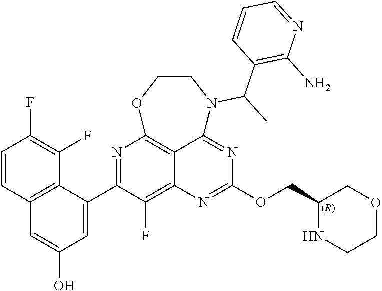 |
| 13 | 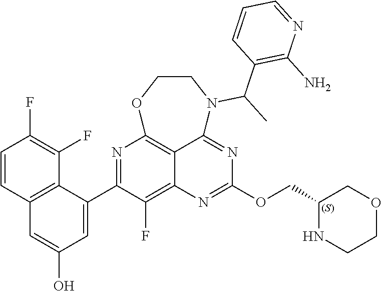 |
| 14 | 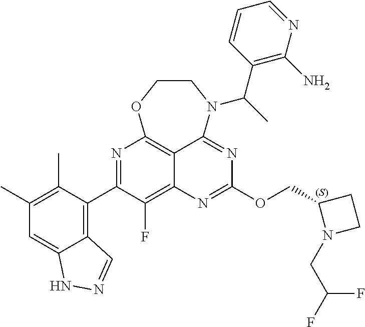 |
| 15 | 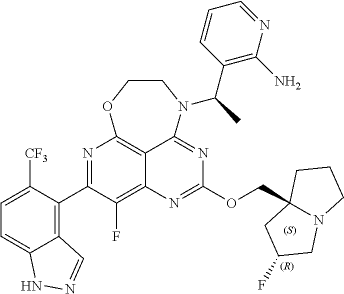 |
| 16 | 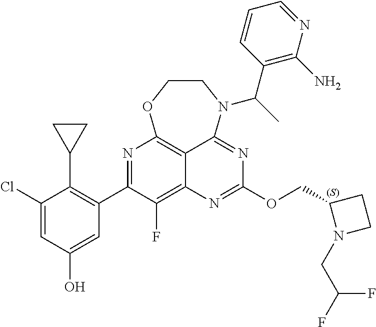 |
| 17 | 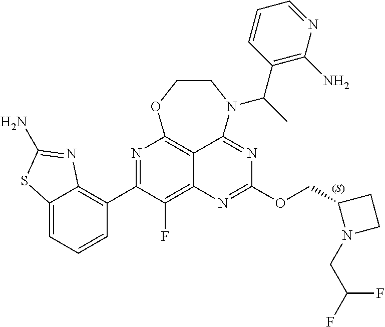 |
| 18 | 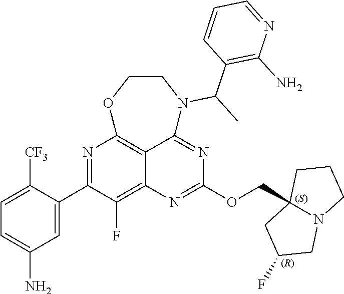 |
| 19 | 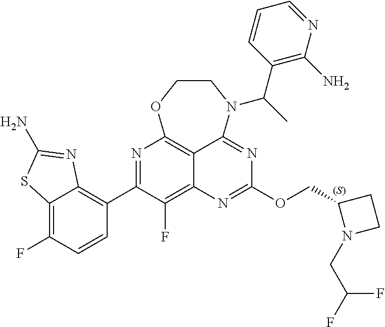 |
| 22 | 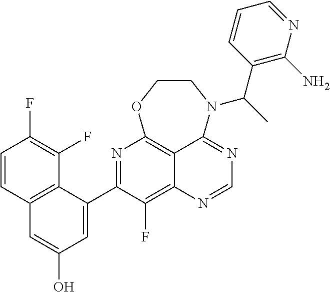 [SMILES: CC(c1cccnc1N)N1CCOc2nc(-c3cc(O)cc4ccc(F)c(F)c34)c(F)c3ncnc1c23] |
| 20 and 21 | 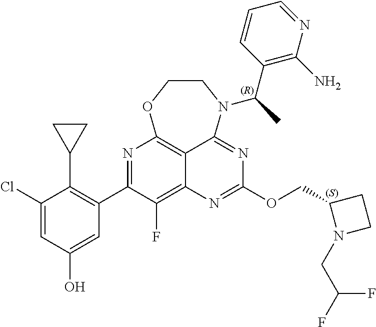 + 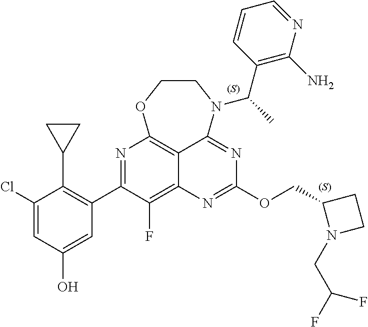 20 & 21 |
| 23 and 24 | 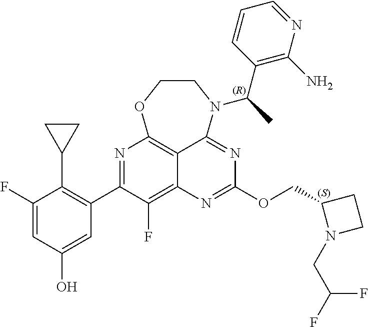 + 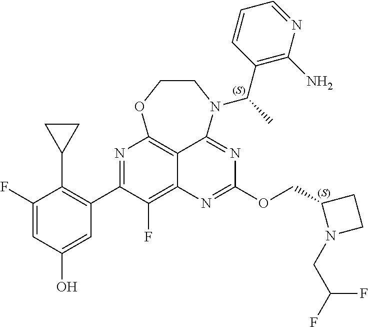 23 & 24 |
| 25 | 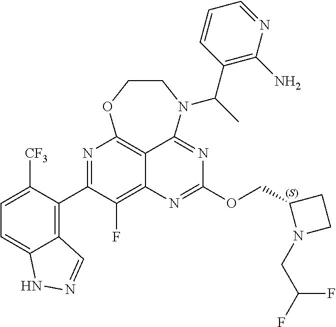 |
| 28 | 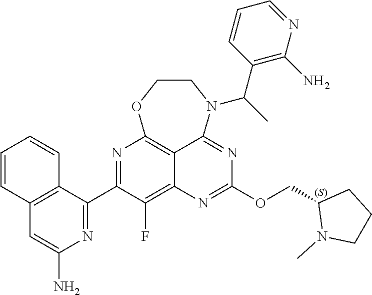 |
| 26 and 27 | 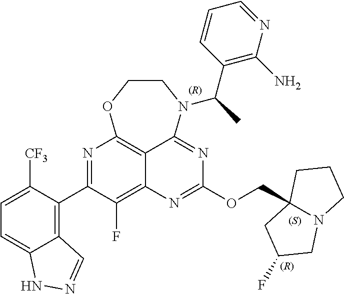 + 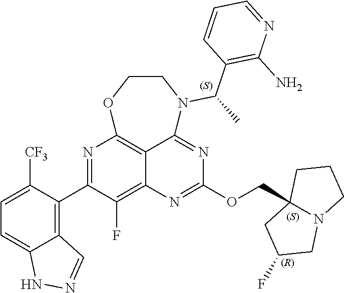 26 & 27 |
| 29 and 30 | 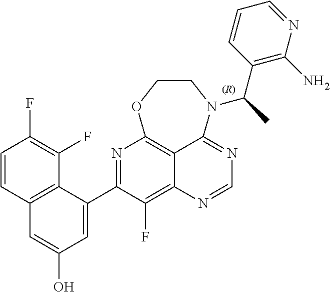 + 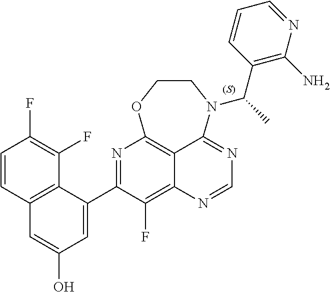 29 & 30 |
| 31 and 32 | 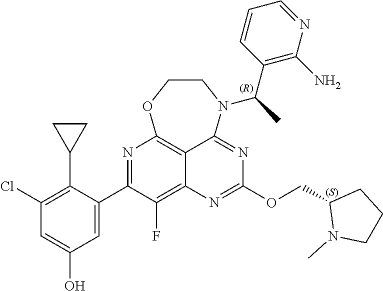 + 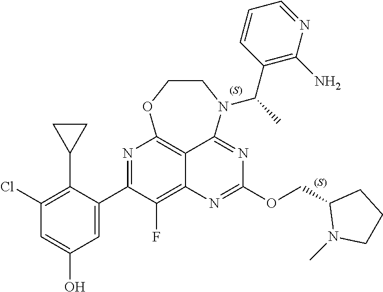 31 & 32 |
| 34 and 35 | 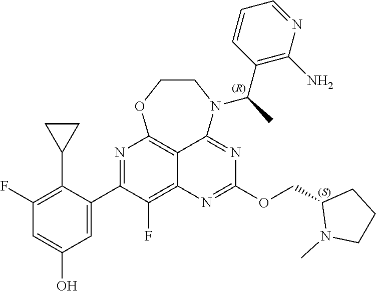 + 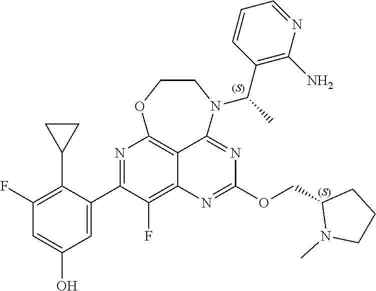 34 & 35 |
| 33 | 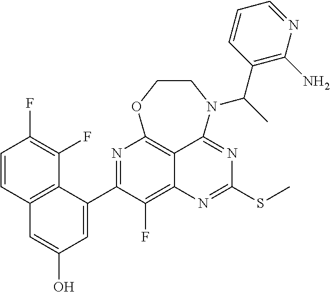 [SMILES: CSc1nc2c3c(nc(-c4cc(O)cc5ccc(F)c(F)c45)c(F)c3n1)OCCN2C(C)c1cccnc1N] |
| 36 | 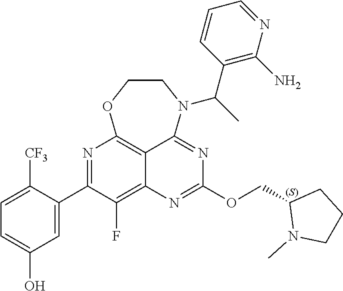 |
| 37 | 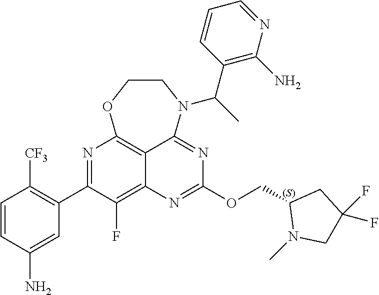 |
| 40 | 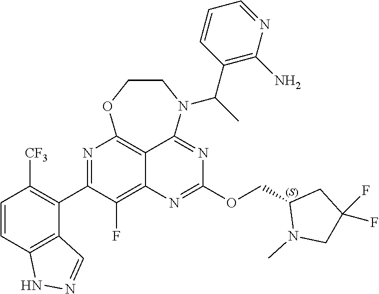 |
| 38 and 39 | 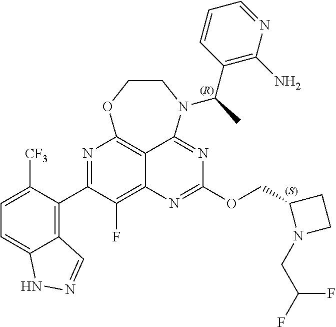 + 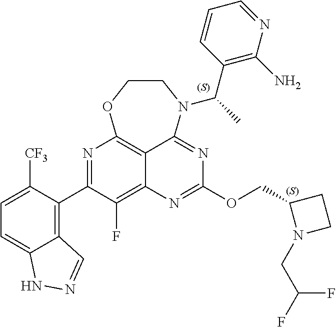 38 & 39 |
| 41 and 42 | 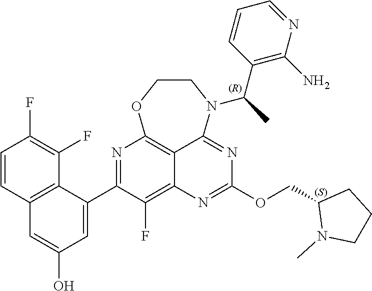 + 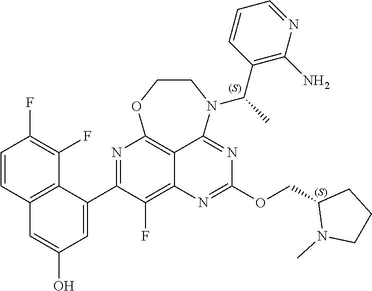 41 & 42 |
| 43 and 44 | 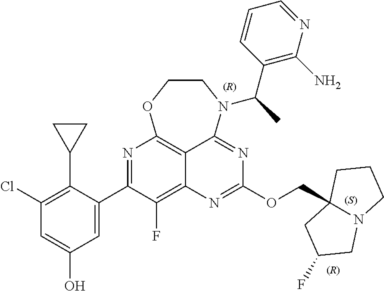 + 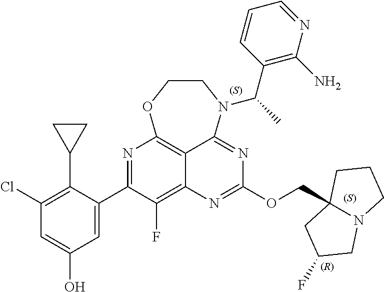 43 & 44 |
| 48 and 49 | 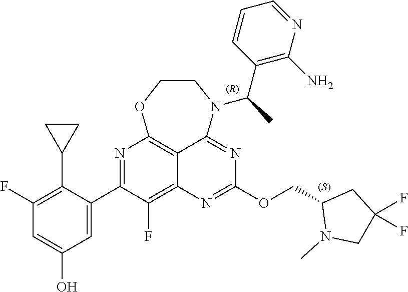 + 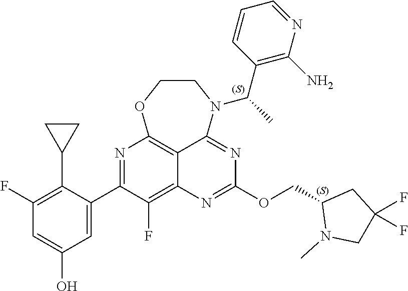 48 & 49 |
| 45 | 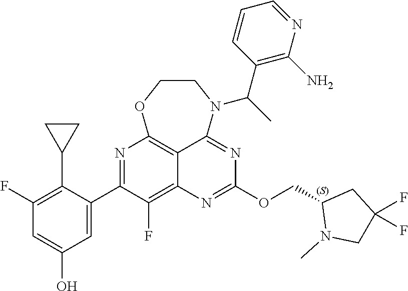 |
| 46 | 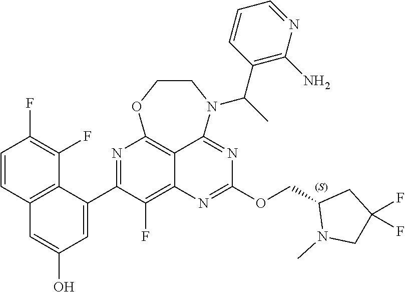 |
| 47 | 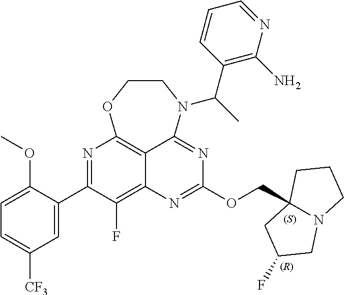 |
| 59 | 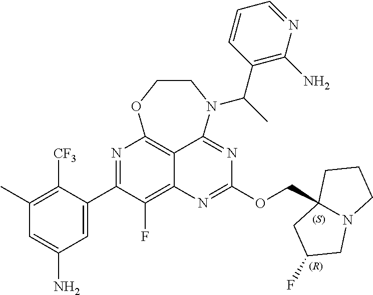 |
| 50 and 51 | 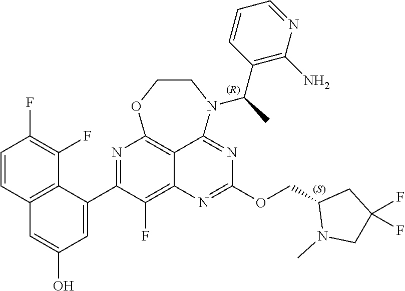 + 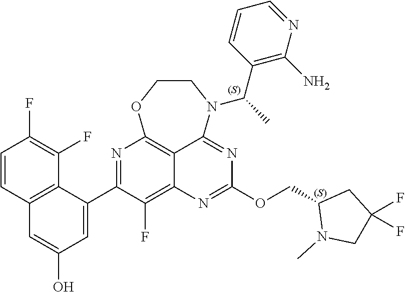 50 & 51 |
| 53 and 54 | 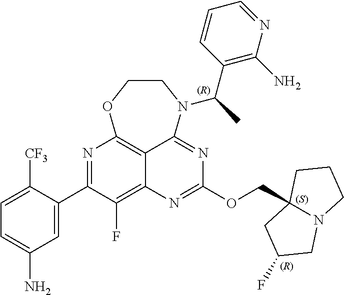 + 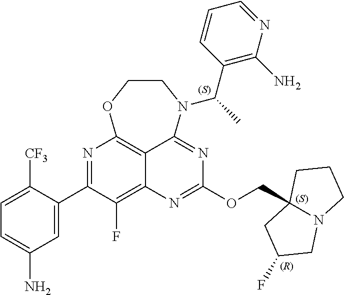 53 & 54 |
| 55 and 56 | 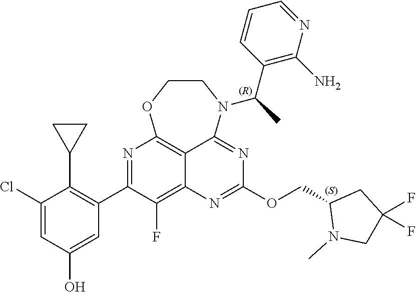 + 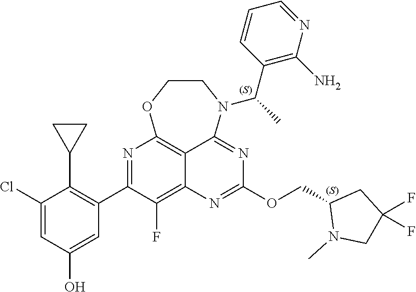 55 & 56 |
| 57 and 58 | 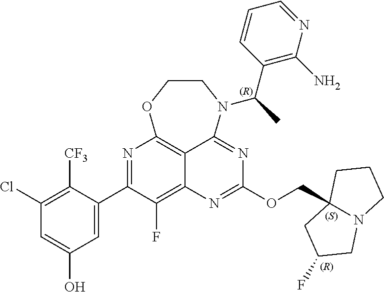 + 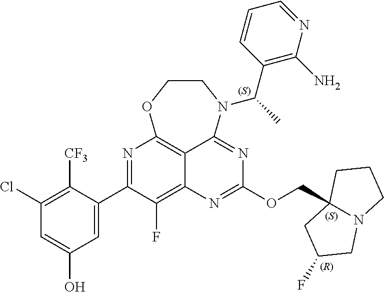 57 & 58 |
| 60 | 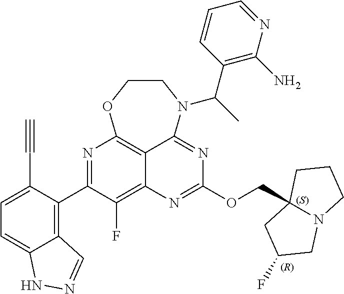 |
| 63 | 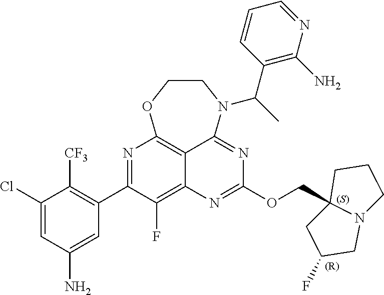 |
| 61 and 62 | 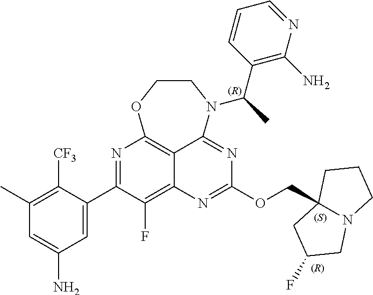 + 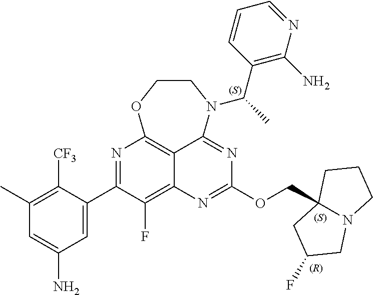 61 & 62 |
| 71 and 72 | 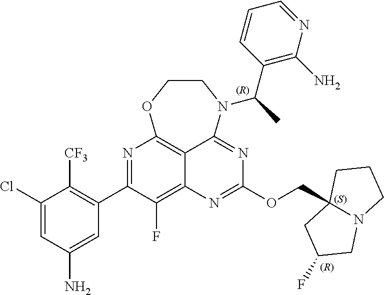 + 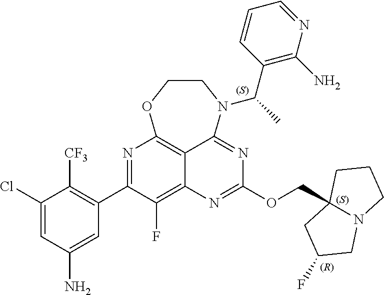 71 & 72 |
| 64 | 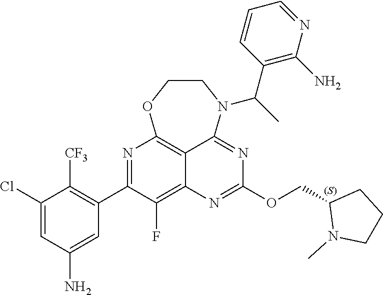 |
| 65 | 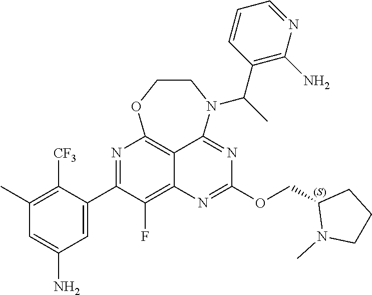 |
| 66 | 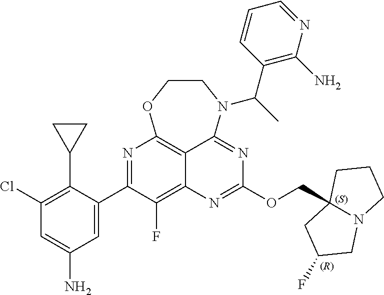 |
| 67 | 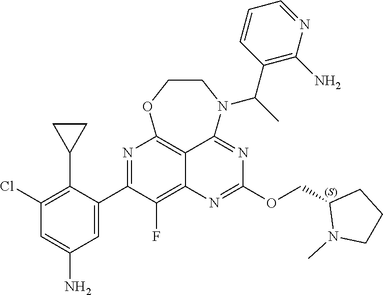 |
| 68 | 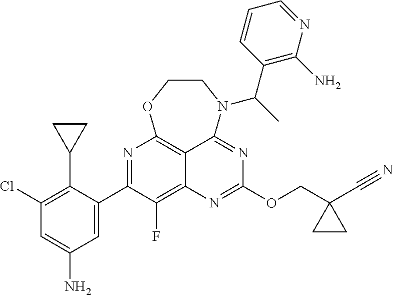 [SMILES: CC(c1cccnc1N)N1CCOc2nc(-c3cc(N)cc(Cl)c3C3CC3)c(F)c3nc(OCC4(C#N)CC4)nc1c23] |
| 69 | 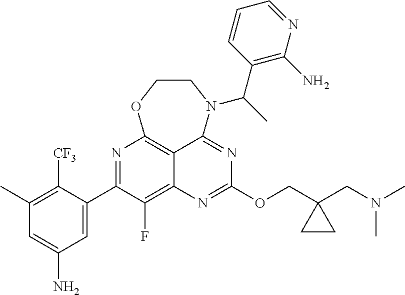 |
| 70 | 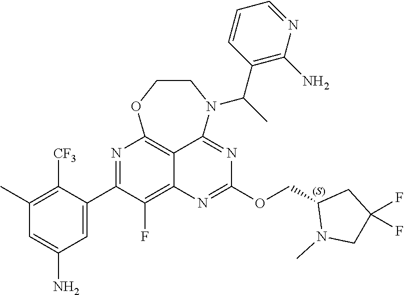 |
| 73 | 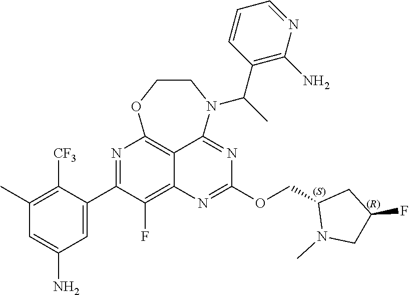 |
| 74 and 75 | 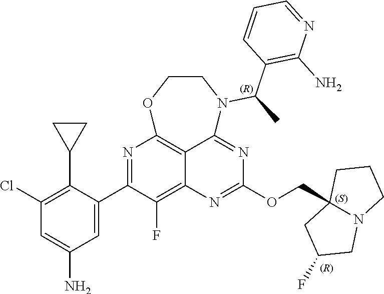 + 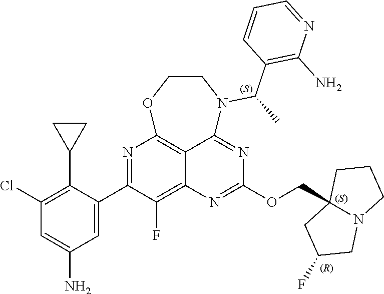 74 & 75 |
| 76 and 77 | 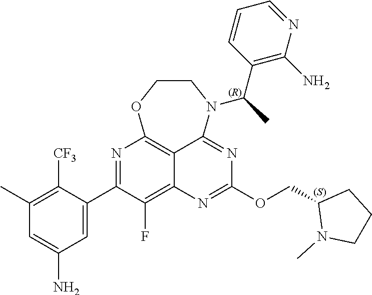 + 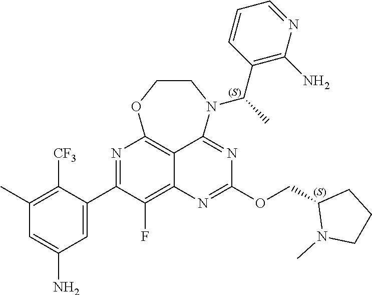 76 & 77 |
| 78 and 79 | 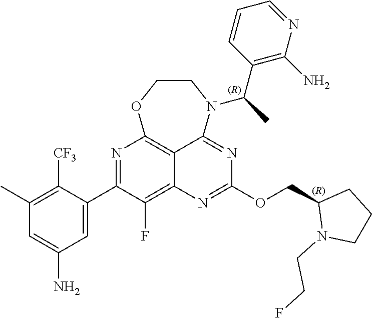 + 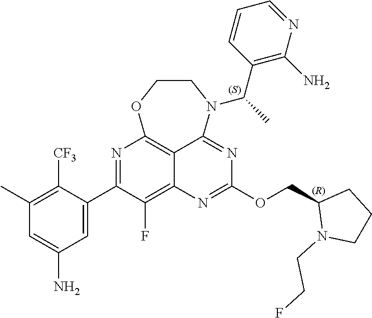 78 & 79 |
| 80 and 81 | 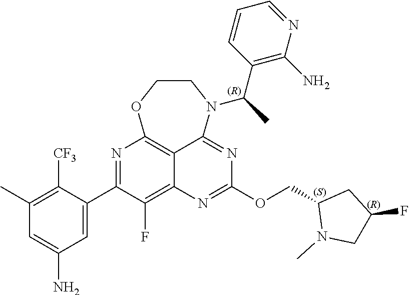 + 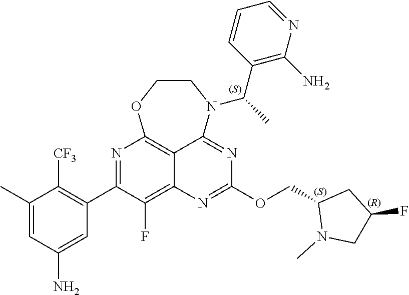 80 & 81 |
| 82 | 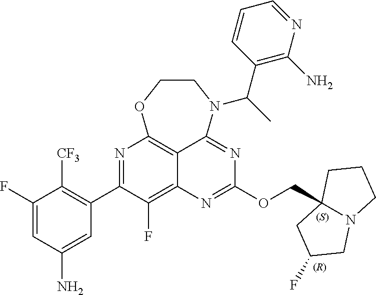 |
| 83 | 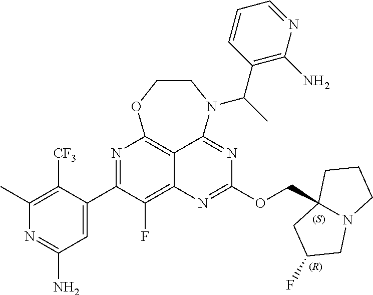 |
| 85 and 86 | 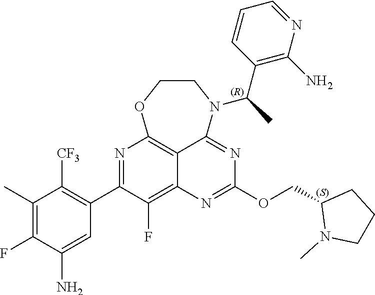 + 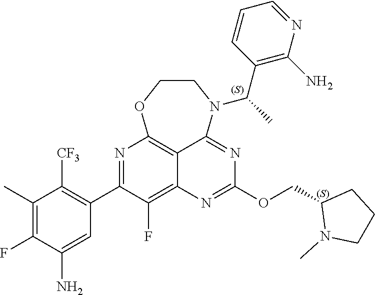 85 & 86 |
| 88 and 89 | 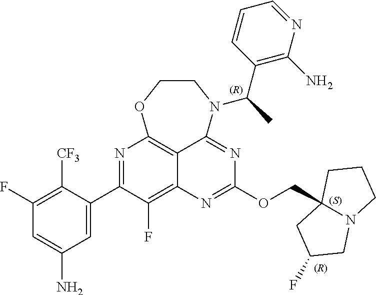 + 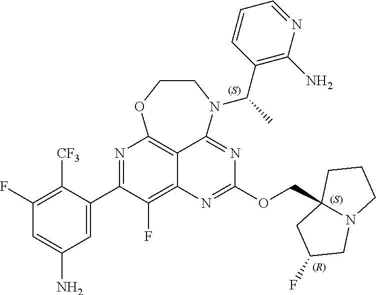 88 & 89 |
| 87 | 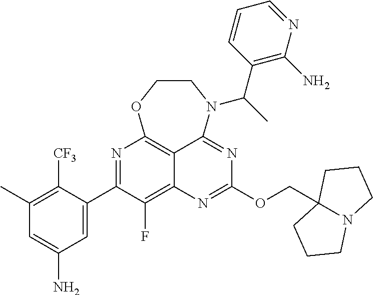 [SMILES: Cc1cc(N)cc(-c2nc3c4c(nc(OCC56CCCN5CCC6)nc4c2F)N(C(C)c2cccnc2N)CCO3)c1C(F)(F)F] |
| 94 | 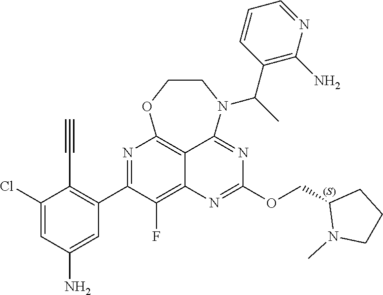 |
| 90 and 91 | 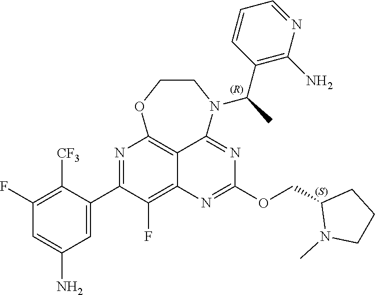 + 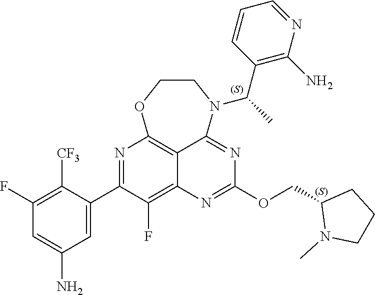 90 & 91 |
| 92 and 93 | 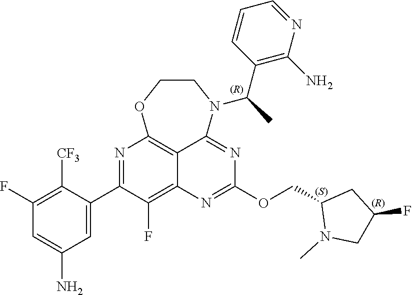 + 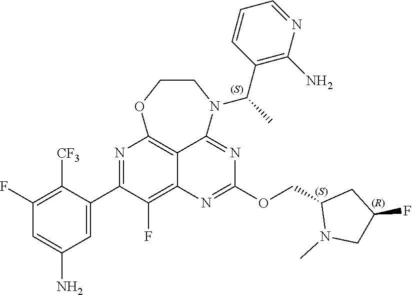 92 & 93 |
| 97 | 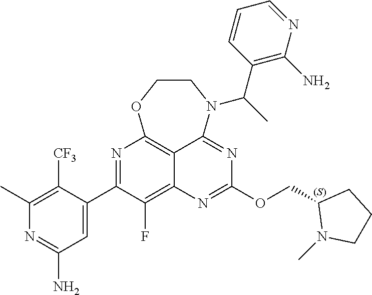 |
| 104 | 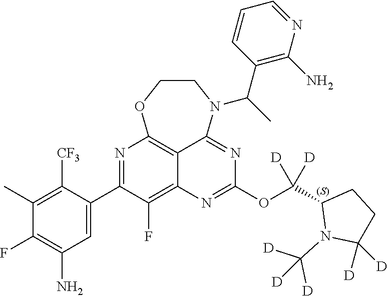 |
| 95 and 96 | 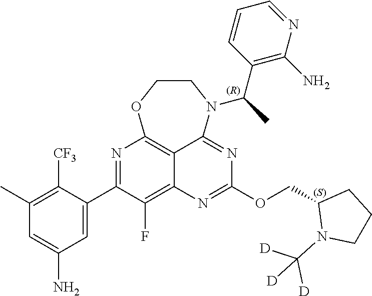 + 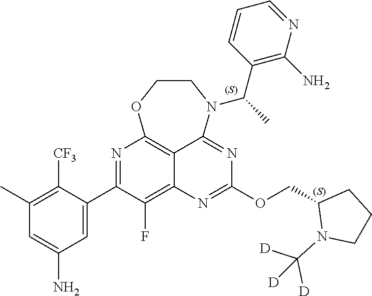 95 & 96 |
| 98 and 99 | 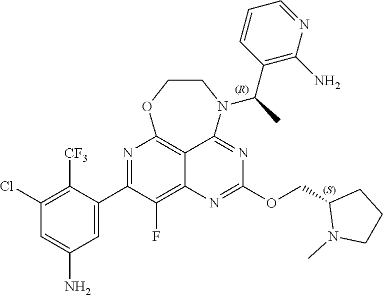 + 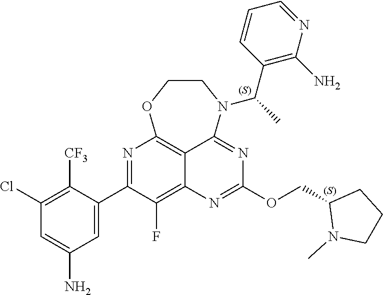 98 & 99 |
| 100 and 101 | 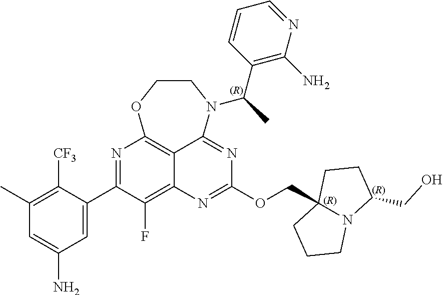 + 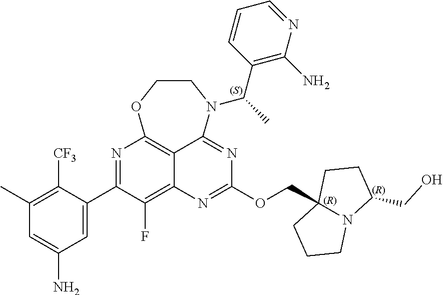 100 & 101 |
| 102 and 103 | 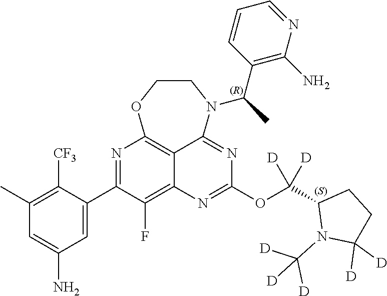 + 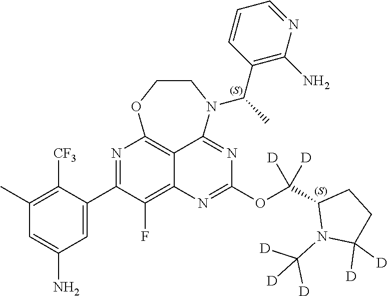 102 & 103 |
| 105 | 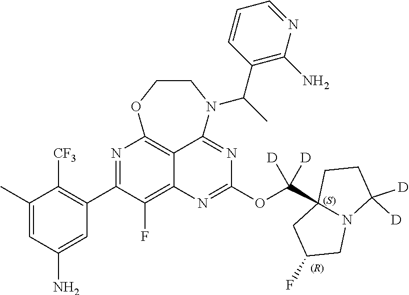 |
| 108 | 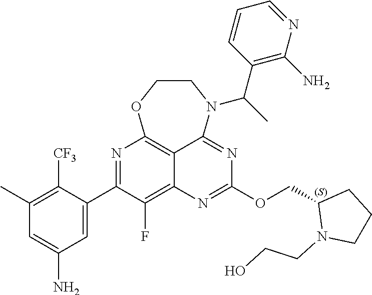 |
| 106 and 107 | 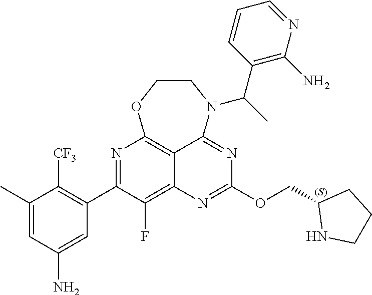 + 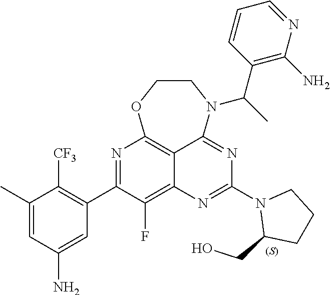 |
| 109 and 110 | 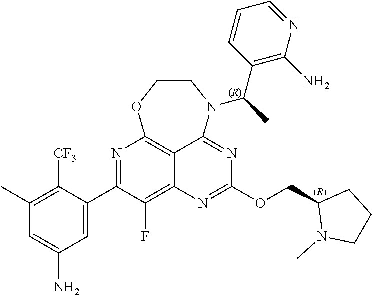 + 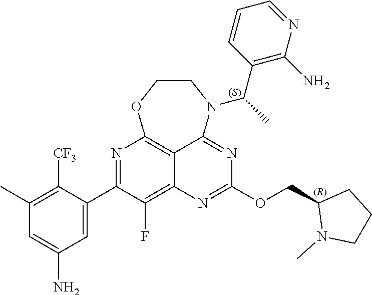 109 & 110 |
| 111 | 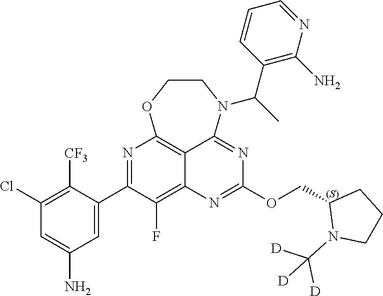 |
| 112 | 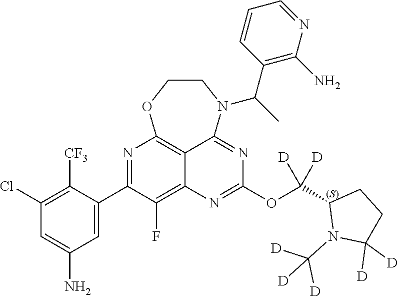 |
| 113 | 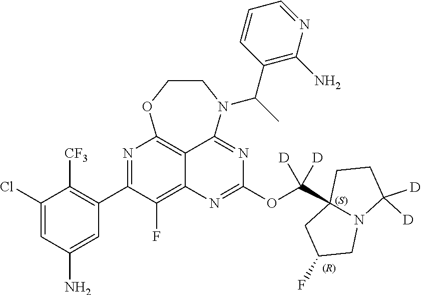 |
| 114 | 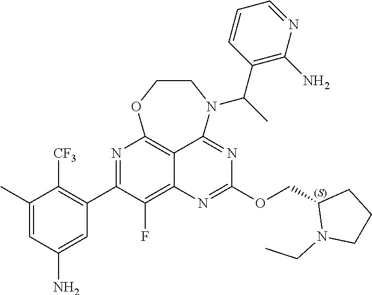 |
| 119 | 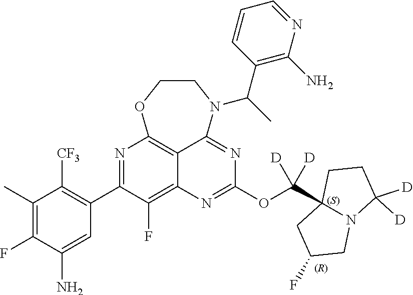 |
| 120 | 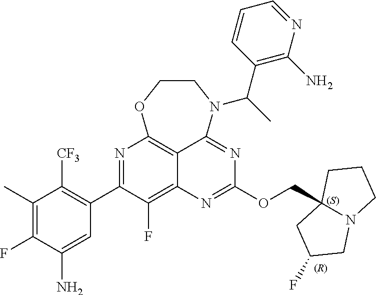 |
| 115 and 116 | 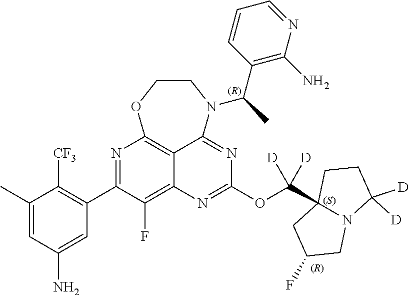 + 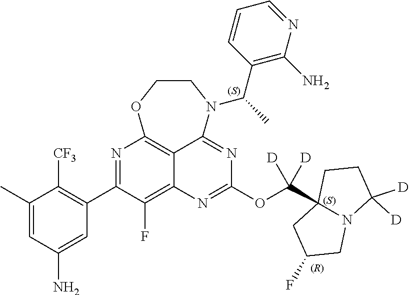 115 & 116 |
| 117 and 118 | 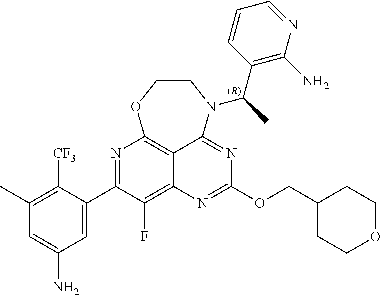 + 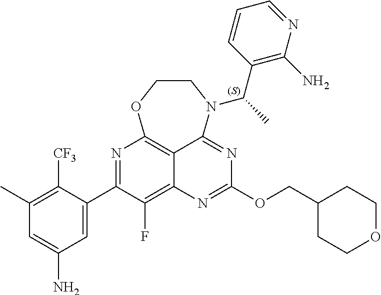 117 & 118 |
| 121 | 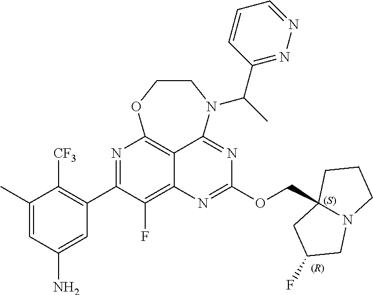 |
| 122 | 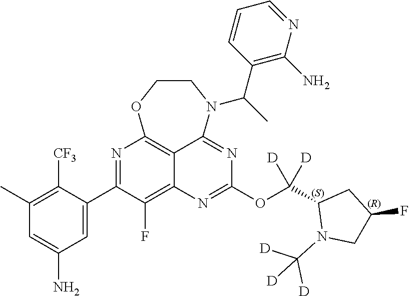 |
| 123 | 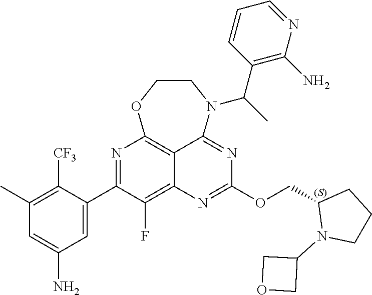 |
| 126 | 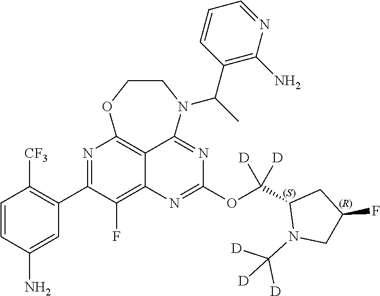 |
| 124 and 125 | 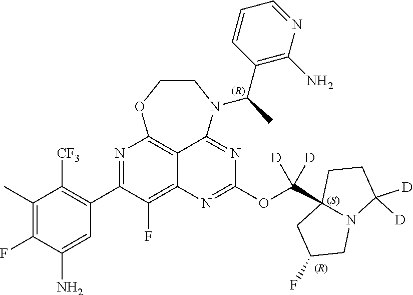 + 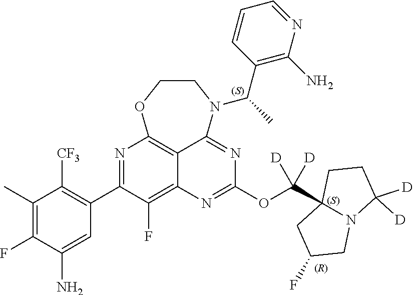 124 & 125 |
| 127 and 128 | 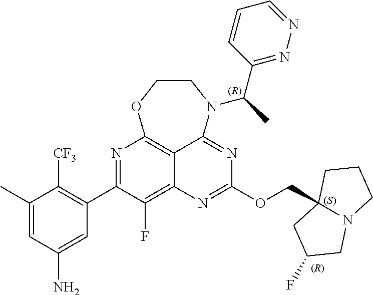 + 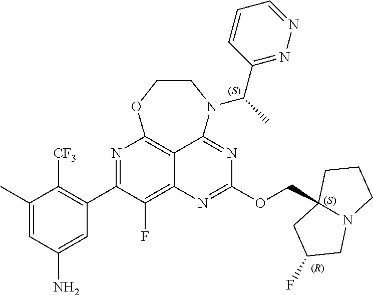 127 & 128 |
| 129 | 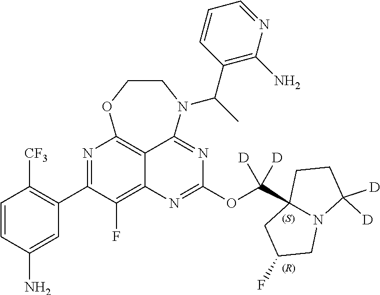 |
| 134 | 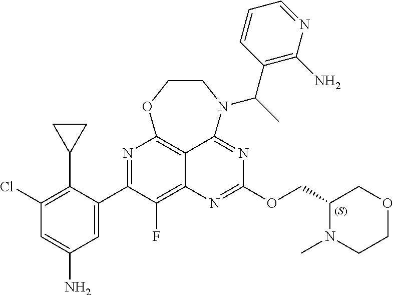 |
| 130 and 131 | 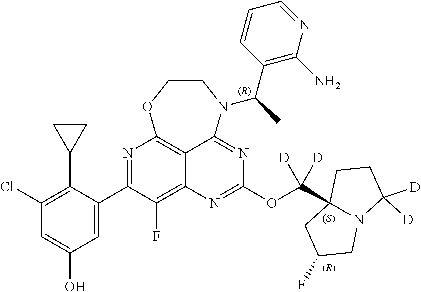 + 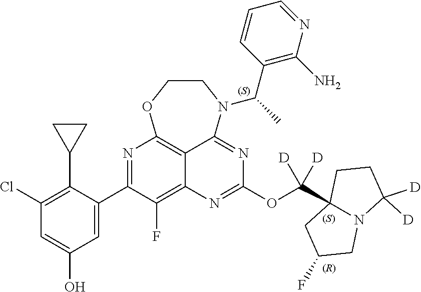 130 & 131 |
| 132 and 133 | 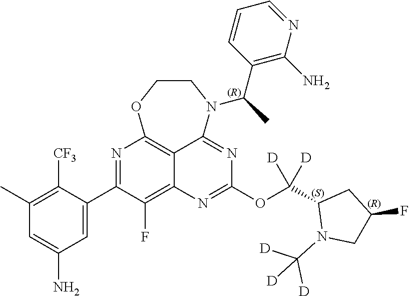 + 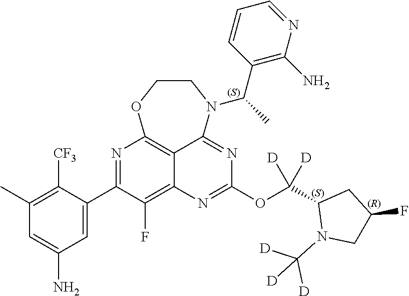 132 & 133 |
| 135 | 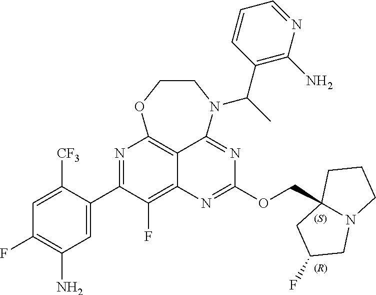 |
| 136 | 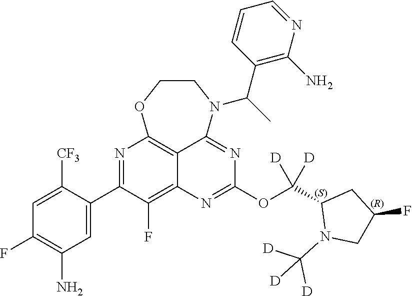 |
| 139 | 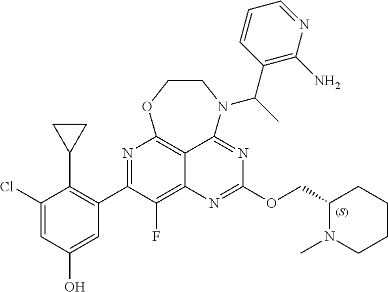 |
| 140 | 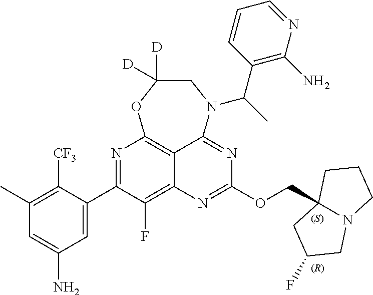 |
| 137 and 138 | 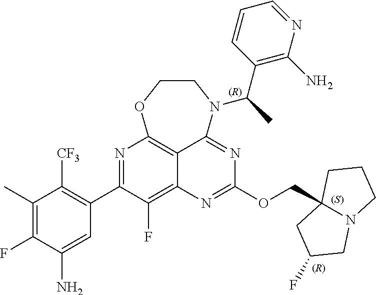 + 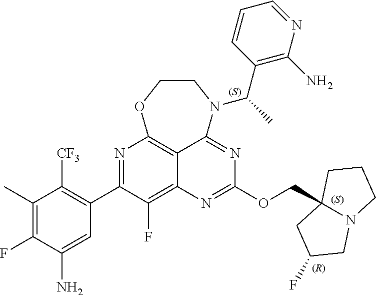 137 & 138 |
| 142 and 143 | 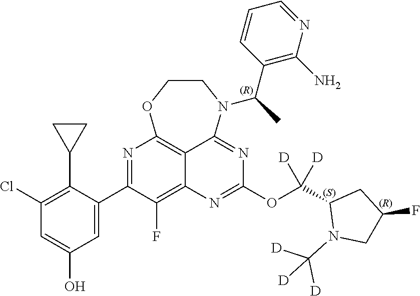 + 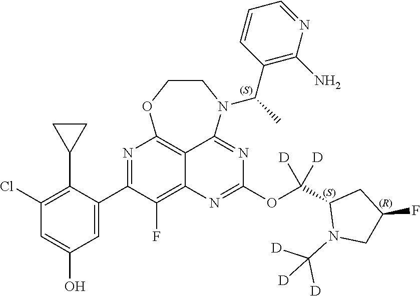 142 & 143 |
| 141 | 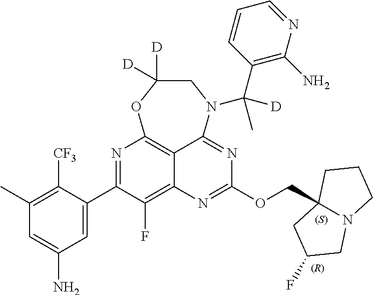 |
| 146 | 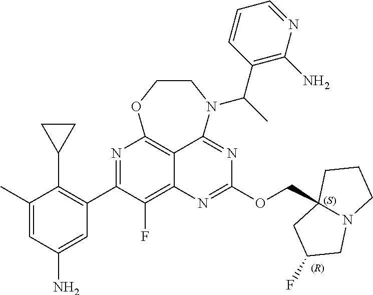 |
| 144 and 145 | 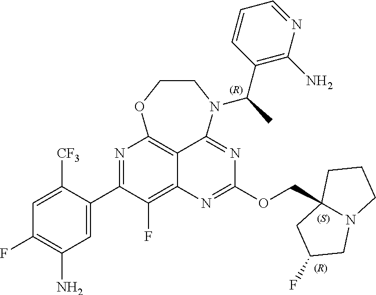 + 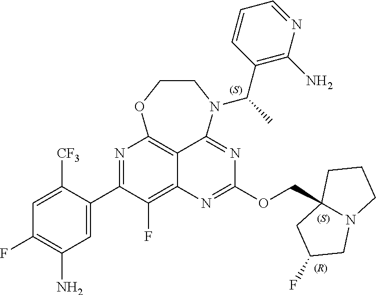 144 & 145 |
| 169 and 170 | 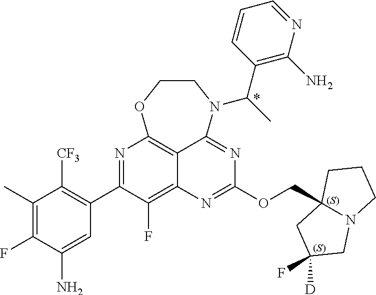 + 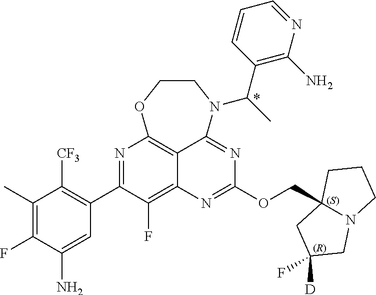 169 & 170 |
| 153 and 154 | 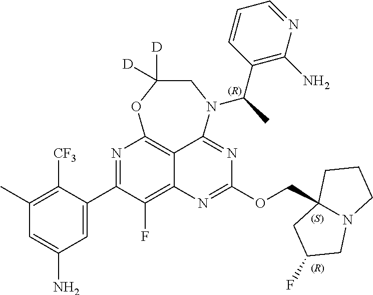 + 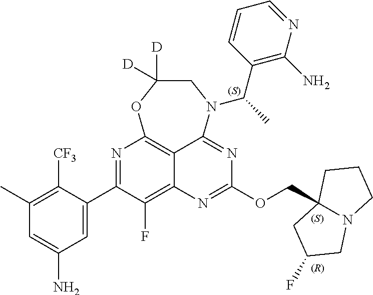 153 & 154 |
| 148 | 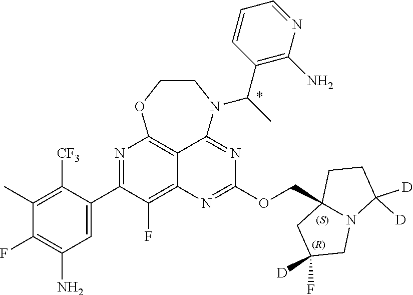 |
| 149 | 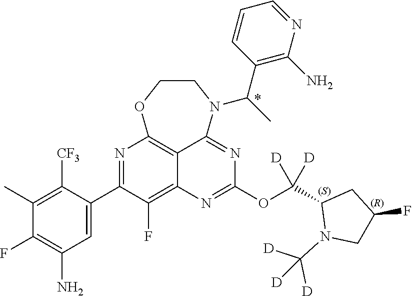 |
| 150 | 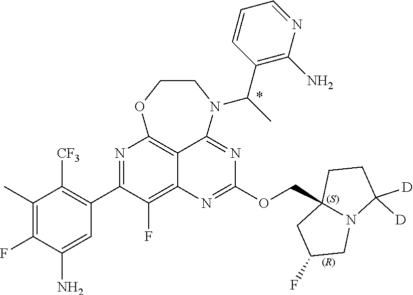 |
| 157 | 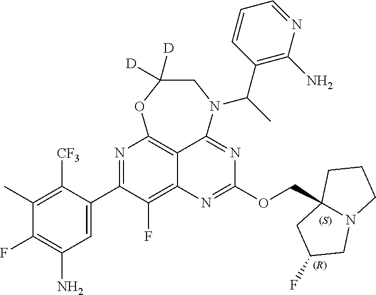 |
| 158 | 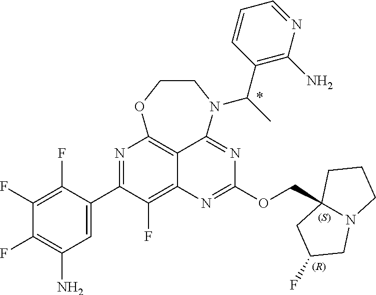 |
| 159 | 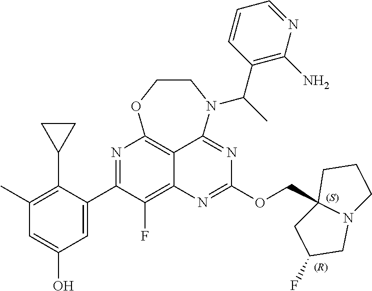 |
| 162 | 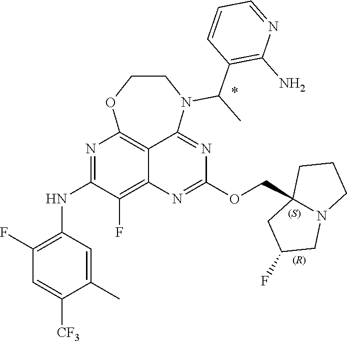 |
| 160 and 161 | 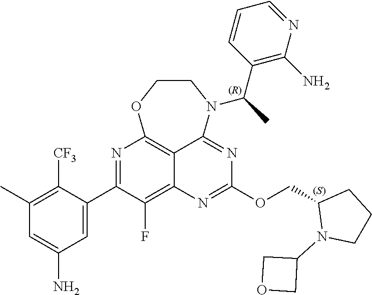 + 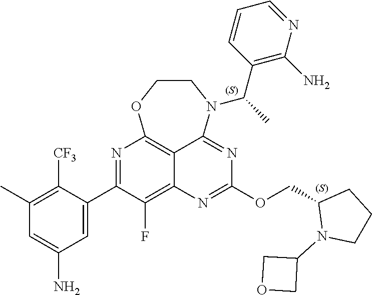 160 & 161 |
| 166 and 167 | 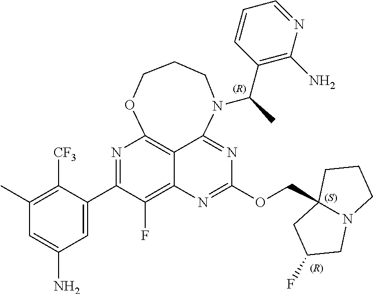 + 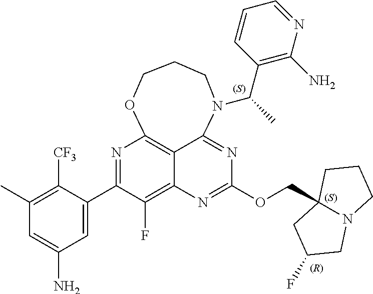 166 & 167 |
| 163 | 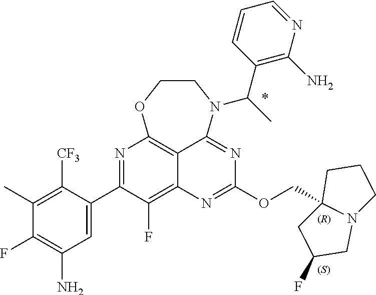 |
| 164 | 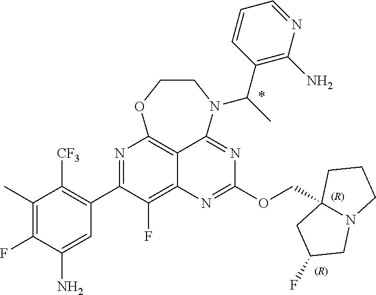 |
| 165 | 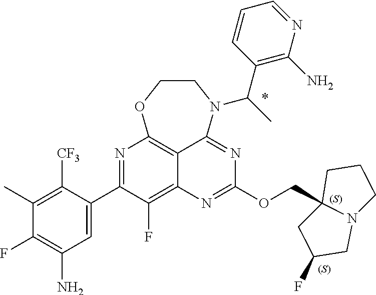 |
| 168 | 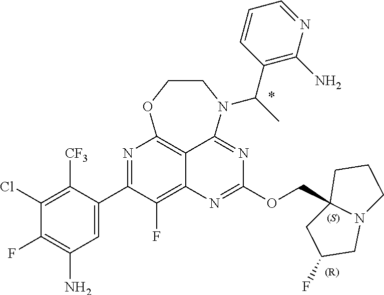 |
| 171 | 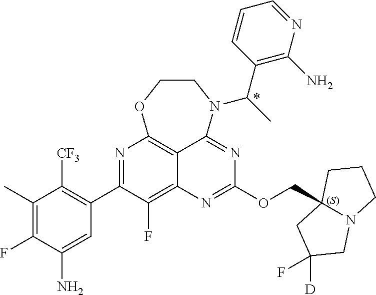 |
| 173 | 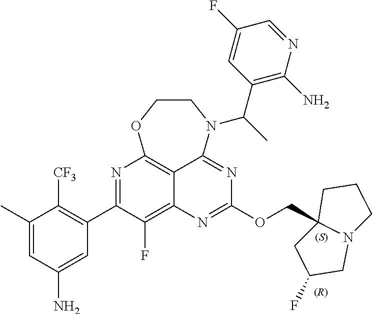 |
| 174 | 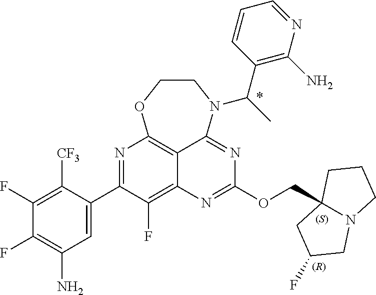 |
| 177 | 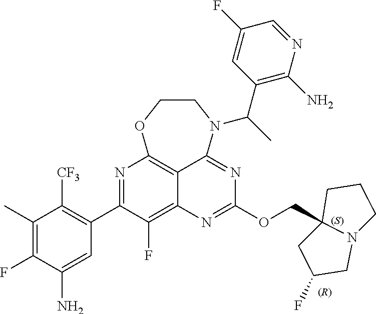 |
| 180 | 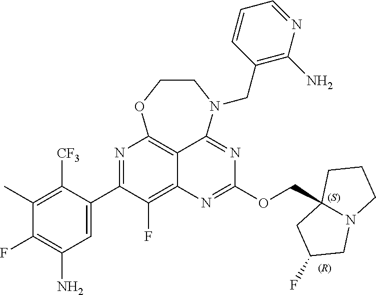 |
| 181 | 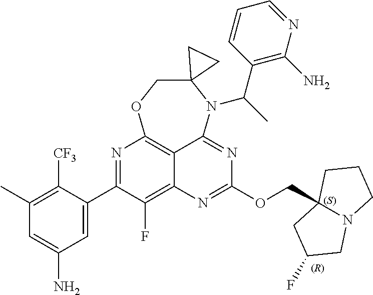 |
| 178 and 179 | 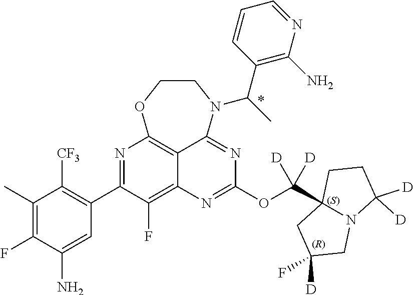 + 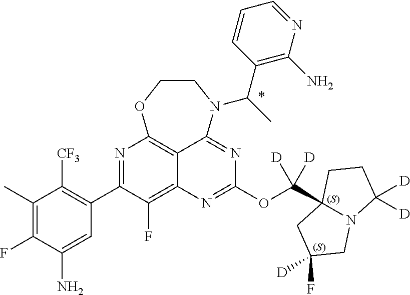 178 & 179 |
| 183 and 184 | 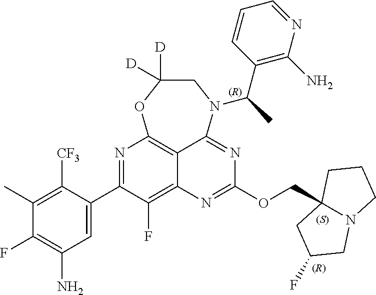 + 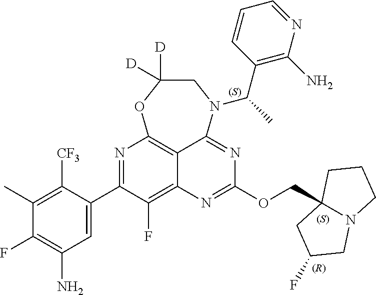 183 & 184 |
| 185 | 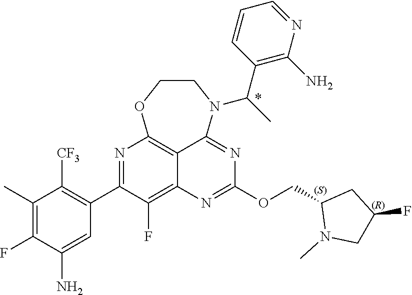 |
| 187 | 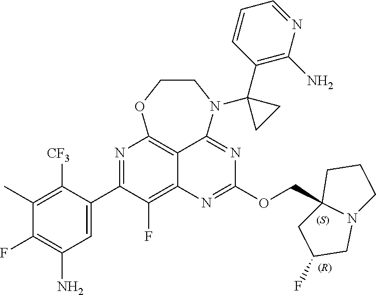 |
| 198 | 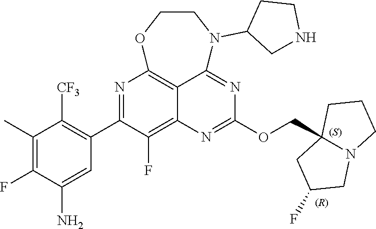 |
| 195 | 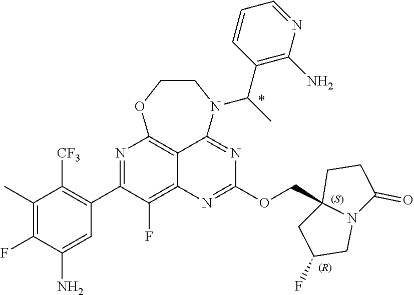 |
| 196 | 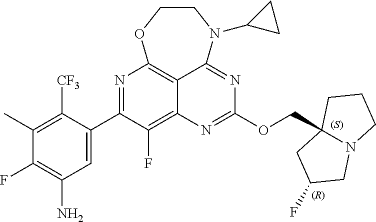 |
| 197 | 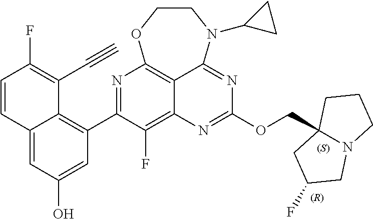 |
| 199 and 200 | 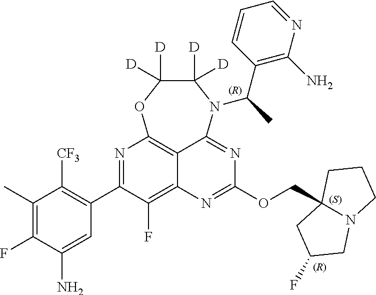 + 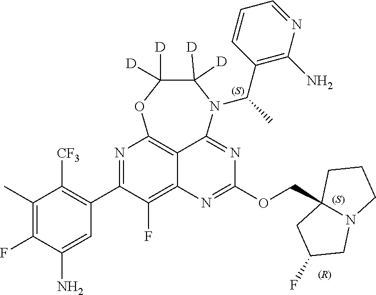 199 & 200 |
| 202 and 203 | 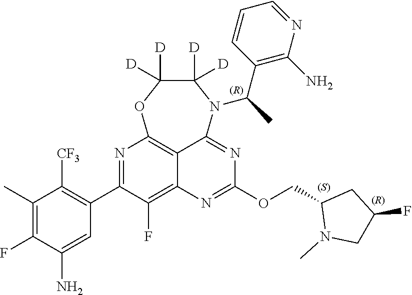 + 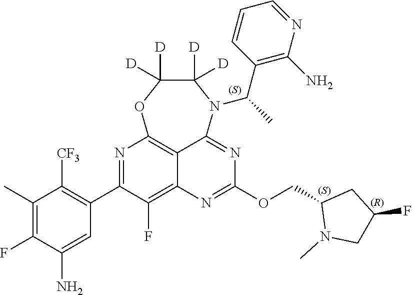 202 & 203 |
| 204 | 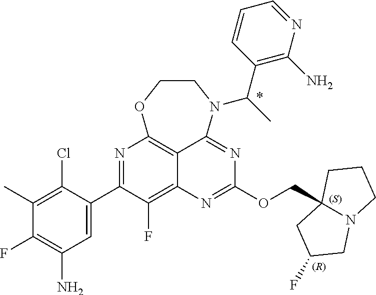 |
| 205 | 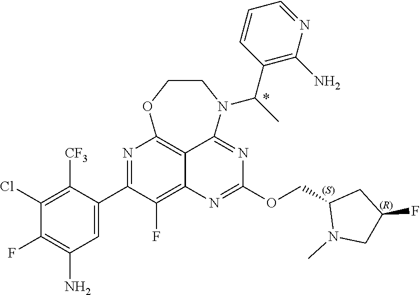 |
| 206 and 207 | 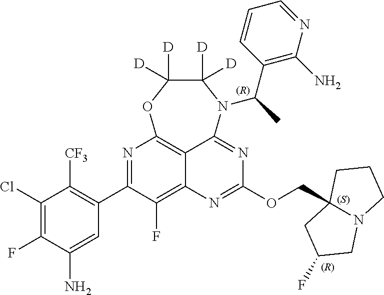 + 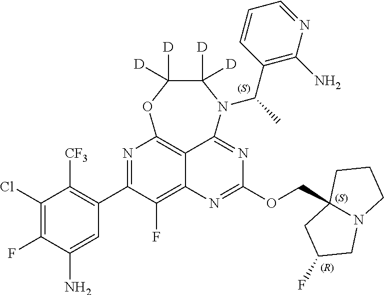 206 & 207 |
| 208 and 209 | 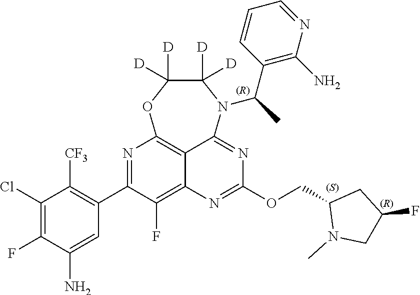 + 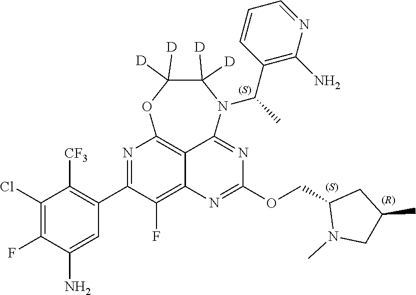 208 & 209 |
| 210 and 211 | 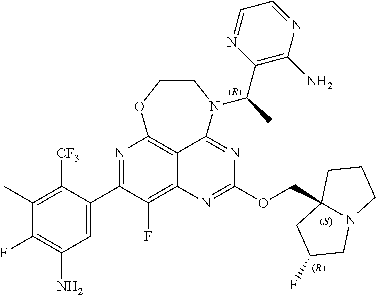 + 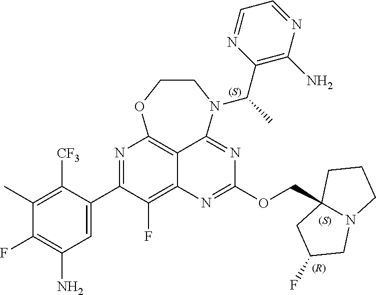 210 & 211 |
| 212 and 213 | 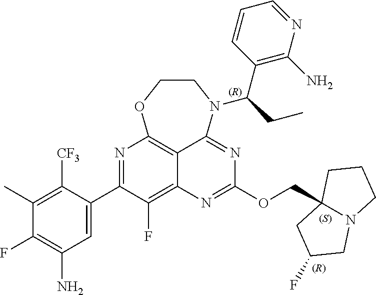 + 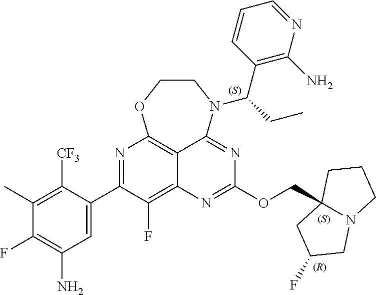 212 & 213 |
| 214 and 215 | 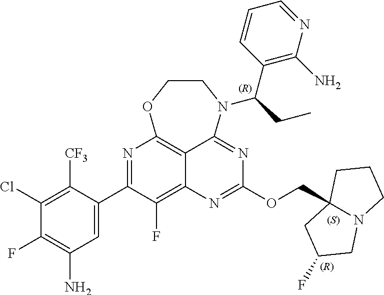 + 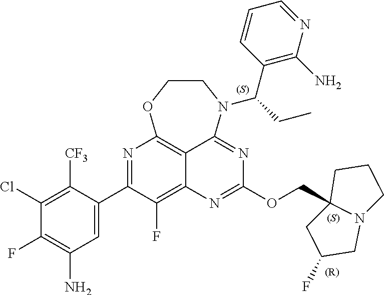 214 & 215 |
| 216 and 217 | 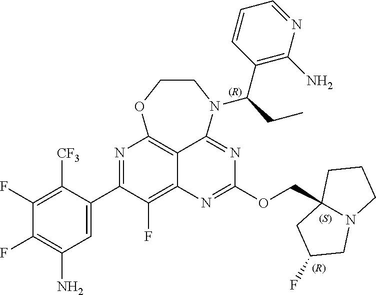 + 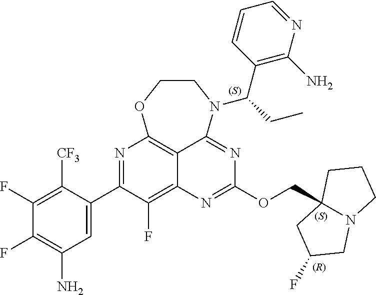 216 & 217 |
| 218 | 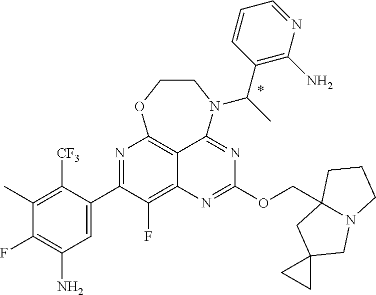 [SMILES: Cc1c(F)c(N)cc(-c2nc3c4c(nc(OCC56CCCN5CC5(CC5)C6)nc4c2F)N(C(C)c2cccnc2N)CCO3)c1C(F)(F)F] |
| 219 and 220 | 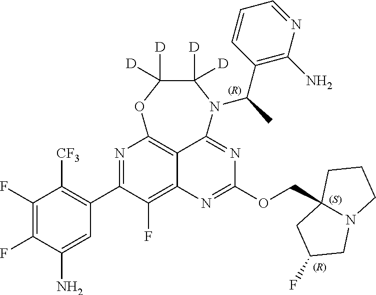 + 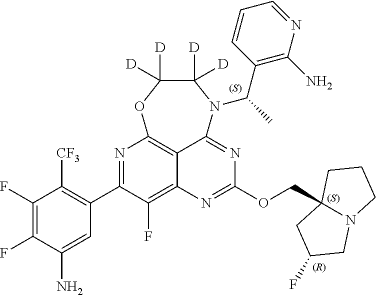 219 & 220 |
| 221 | 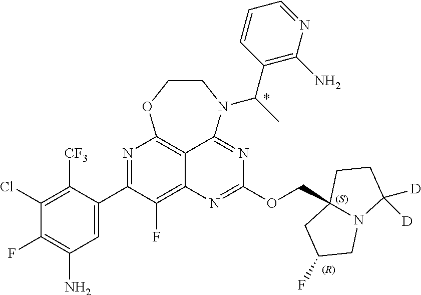 |
| 224 | 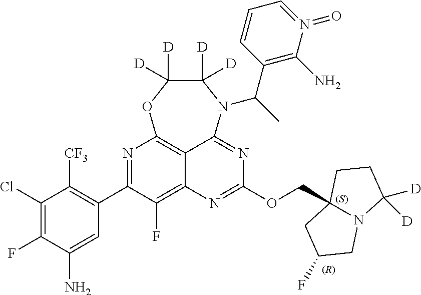 |
| 222 and 223 | 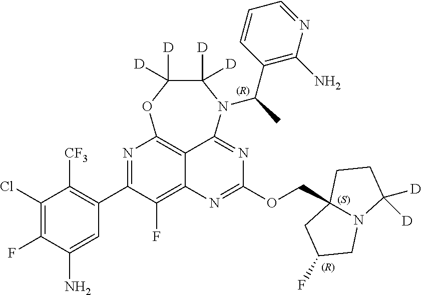 + 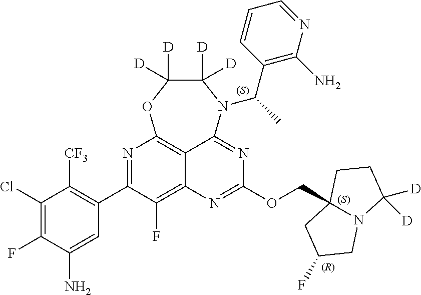 222 & 223 |
| 238 and 239 | 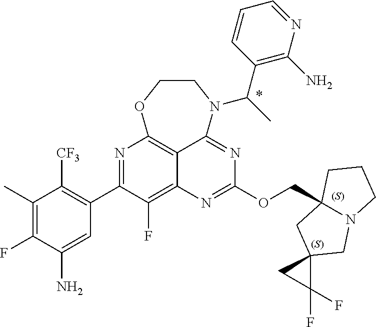 + 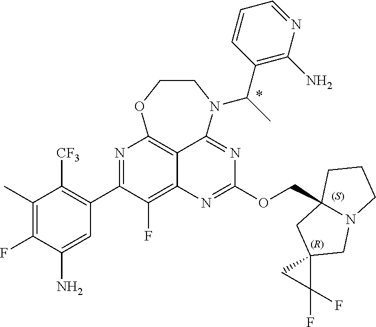 238 & 239 |
| 225 | 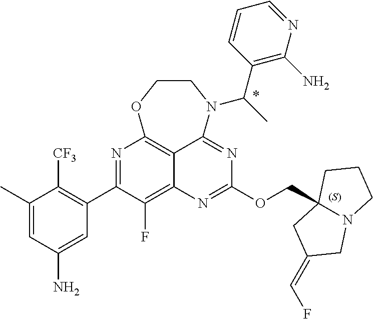 |
| 226 | 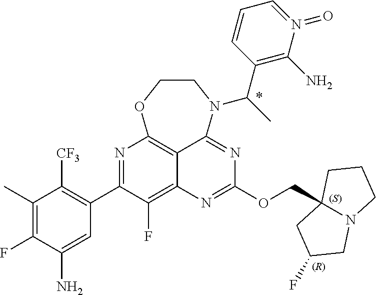 |
| 227 | 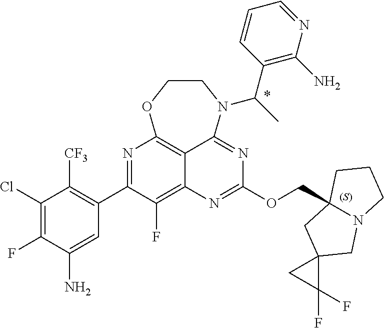 |
| 228 | 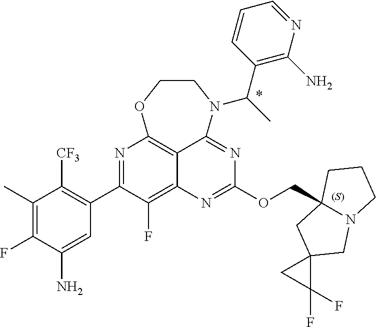 |
| 229 | 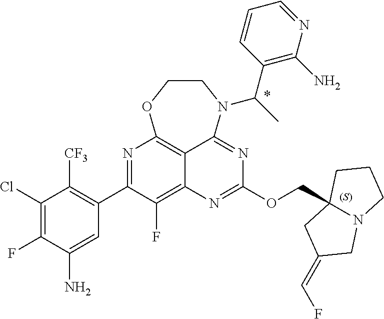 |
| 230 | 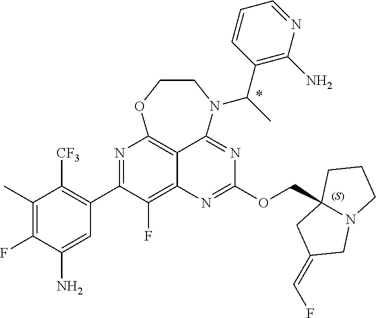 |
| 231 | 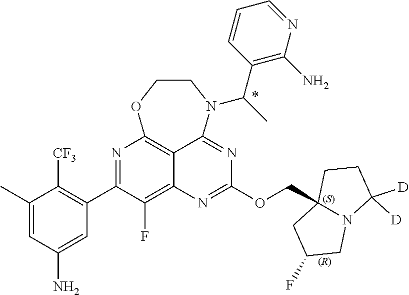 |
| 232 | 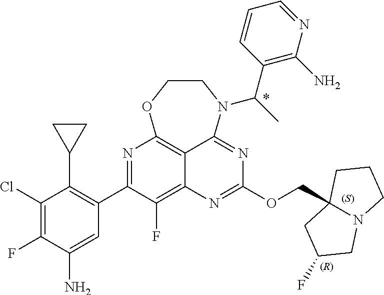 |
| 233 | 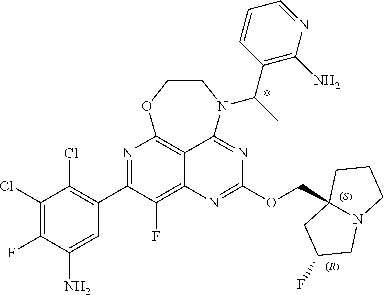 |
| 234 | 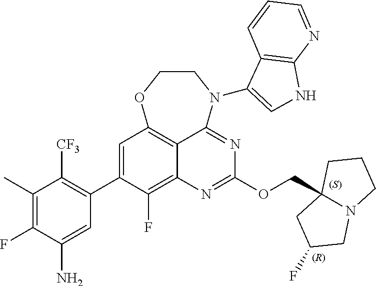 |
| 235 | 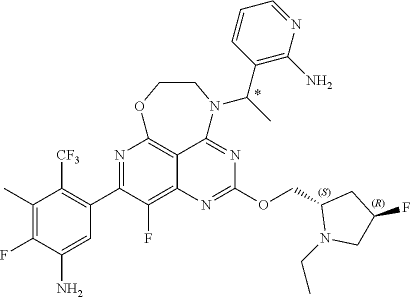 |
| 236 | 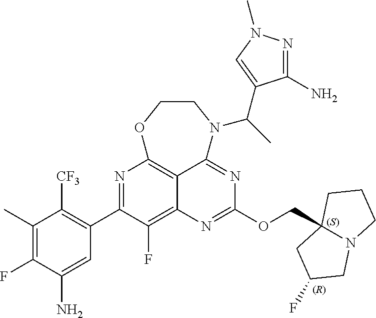 |
| 237 | 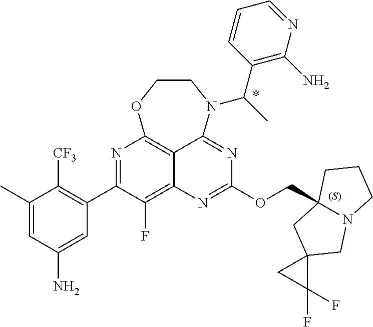 |
| 281 | 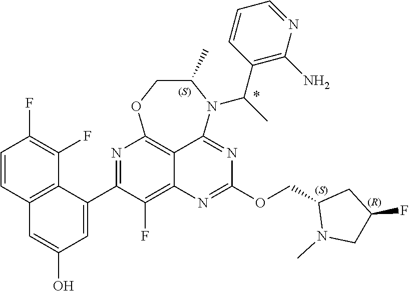 |
| 241 and 242 | 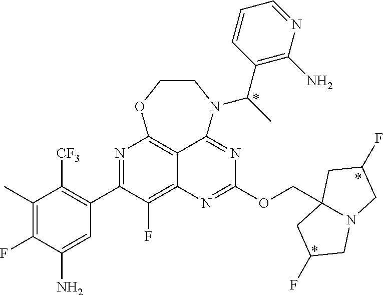 [SMILES: Cc1c(F)c(N)cc(-c2nc3c4c(nc(OCC56CC(F)CN5CC(F)C6)nc4c2F)N(C(C)c2cccnc2N)CCO3)c1C(F)(F)F] + 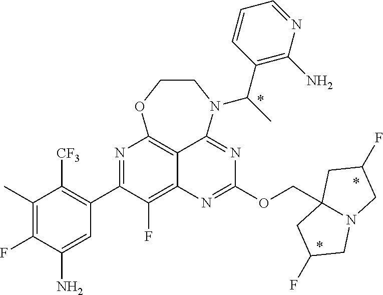 [SMILES: Cc1c(F)c(N)cc(-c2nc3c4c(nc(OCC56CC(F)CN5CC(F)C6)nc4c2F)N(C(C)c2cccnc2N)CCO3)c1C(F)(F)F] 241 & 242 |
| 245 and 246 | 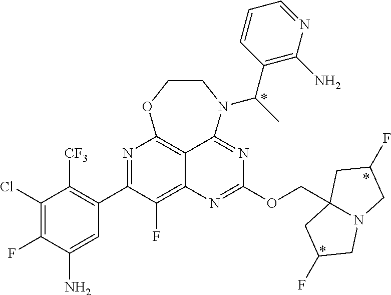 [SMILES: CC(c1cccnc1N)N1CCOc2nc(-c3cc(N)c(F)c(Cl)c3C(F)(F)F)c(F)c3nc(OCC45CC(F)CN4CC(F)C5)nc1c23] + 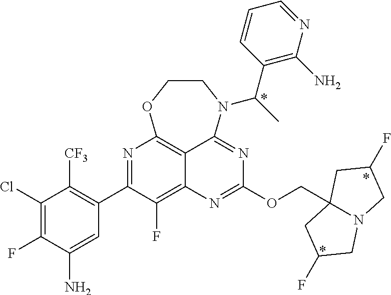 [SMILES: CC(c1cccnc1N)N1CCOc2nc(-c3cc(N)c(F)c(Cl)c3C(F)(F)F)c(F)c3nc(OCC45CC(F)CN4CC(F)C5)nc1c23] 245 & 246 |
| 243 | 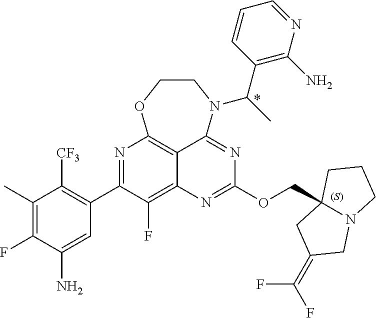 |
| 259 | 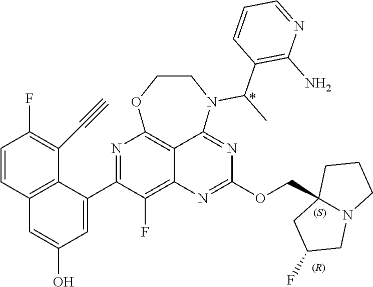 |
| 247 and 248 | 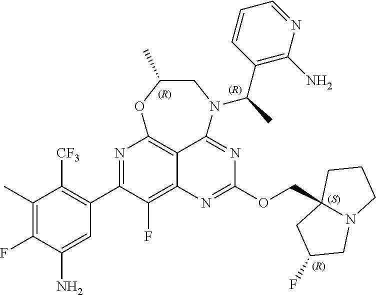 + 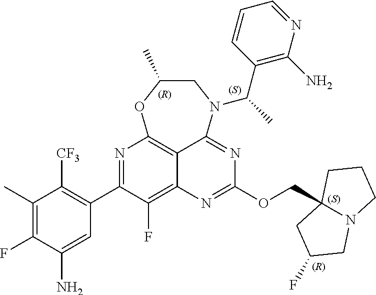 247 & 248 |
| 250 and 263 | 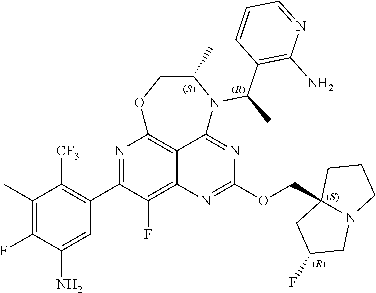 + 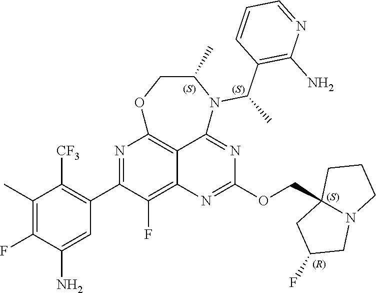 250 & 263 |
| 251 and 252 | 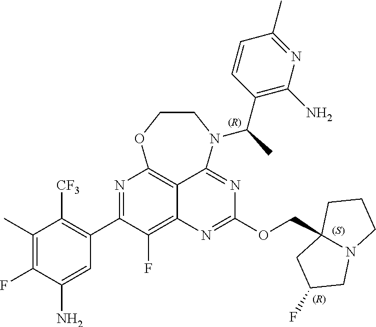 + 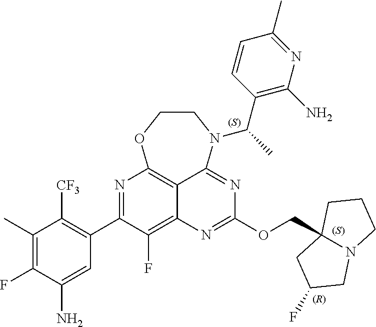 251 & 252 |
| 253 and 254 | 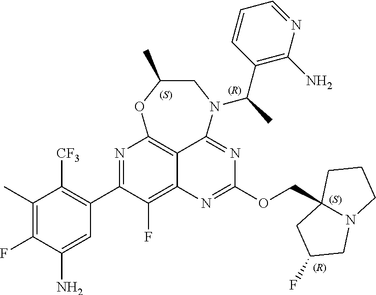 + 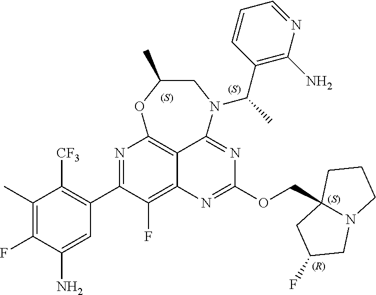 253 & 254 |
| 255 and 256 | 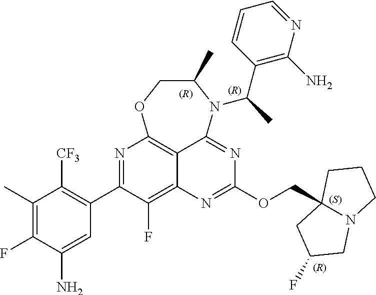 + 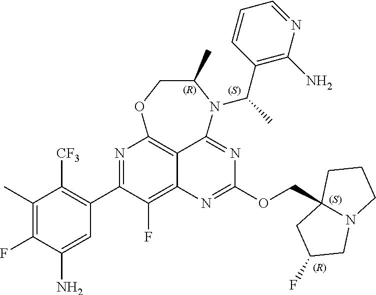 255 & 256 |
| 257 and 258 | 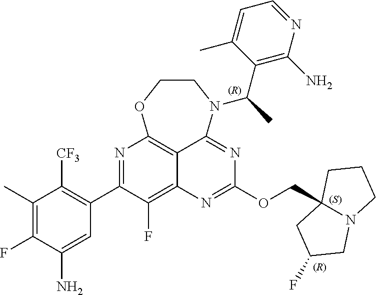 + 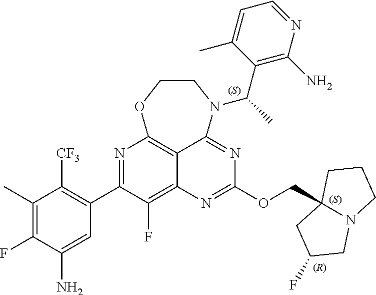 257 & 258 |
| 260 and 261 | 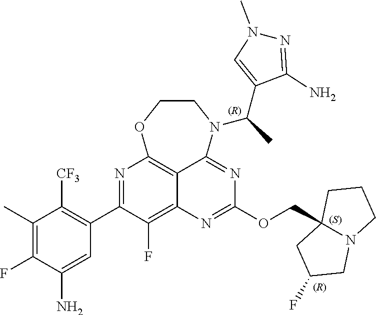 + 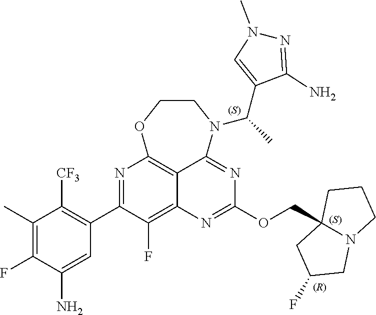 260 & 261 |
| 264 and 265 | 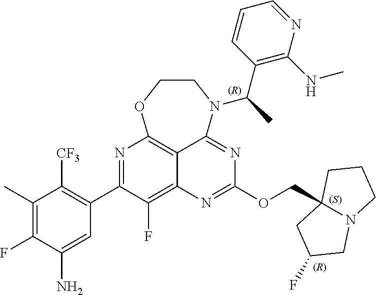 + 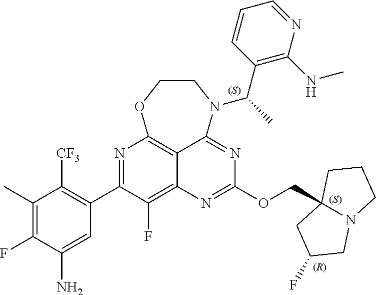 264 & 265 |
| 262 | 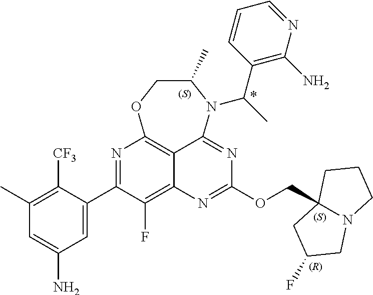 |
| 266 | 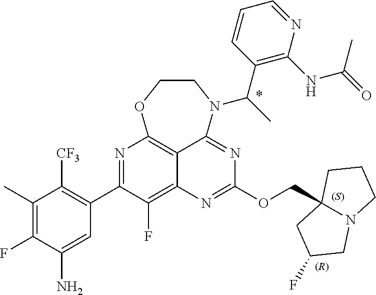 |
| 267 | 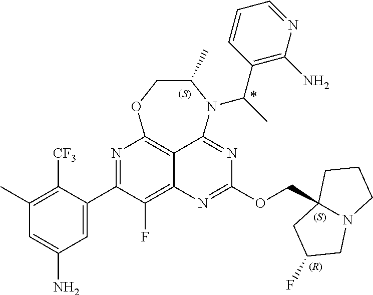 |
| 268 | 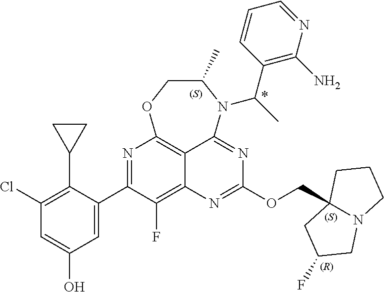 |
| 269 and 272 | 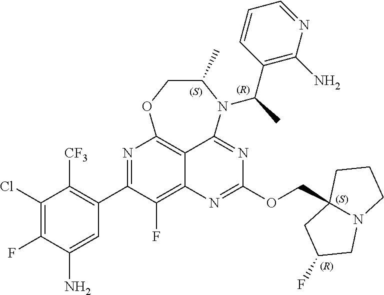 + 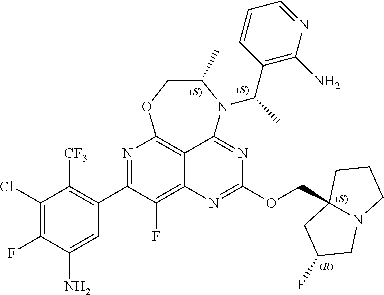 269 & 272 |
| 270 and 271 | 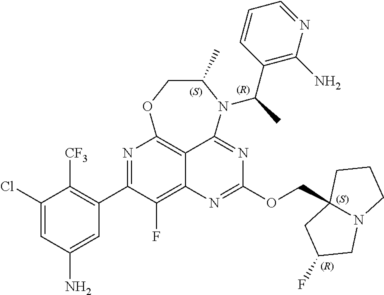 + 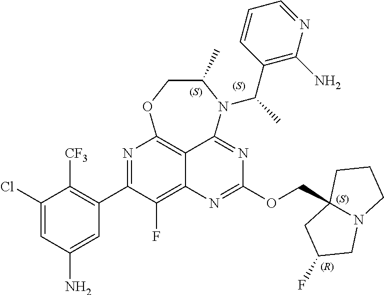 270 & 271 |
| 273 and 274 | 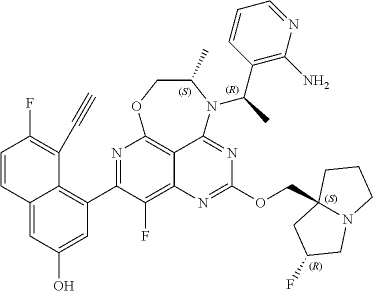 + 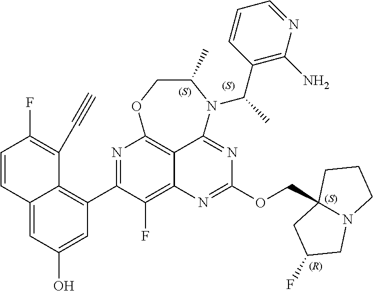 273 & 274 |
| 275 and 276 | 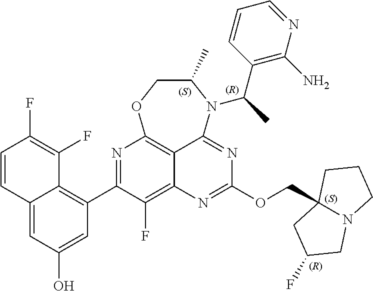 + 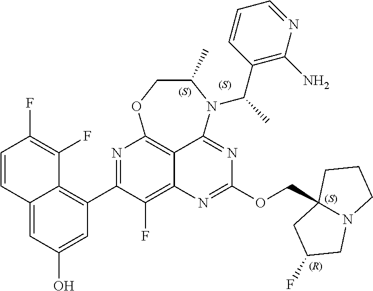 275 & 276 |
| 277 and 278 | 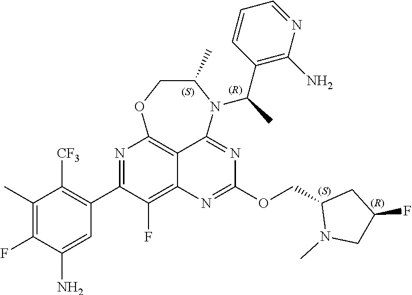 + 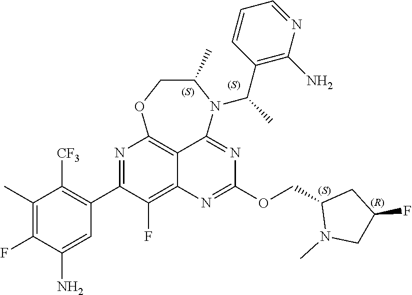 277 & 278 |
| 279 and 280 | 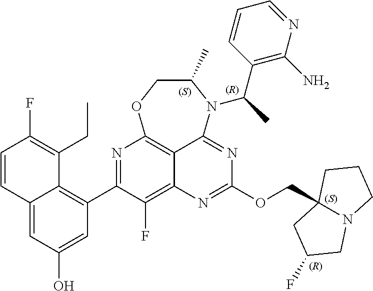 + 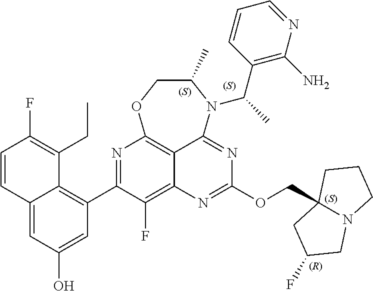 279 & 280 |
| 282 and 283 | 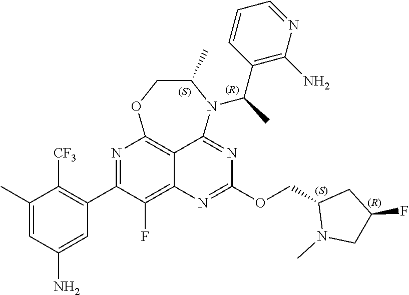 + 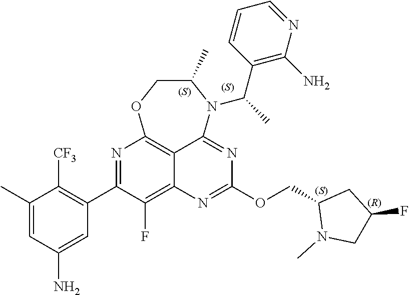 282 & 283 |
| 284 and 285 | 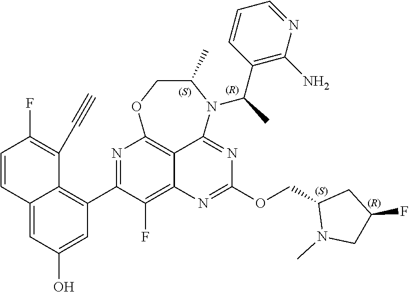 + 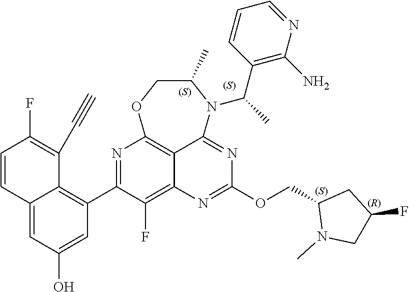 284 & 285 |
| 286 and 287 | 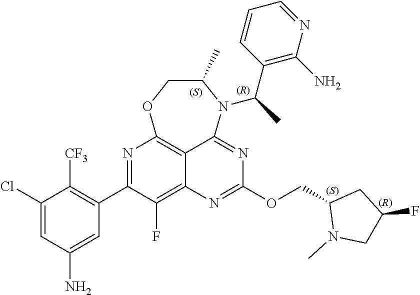 + 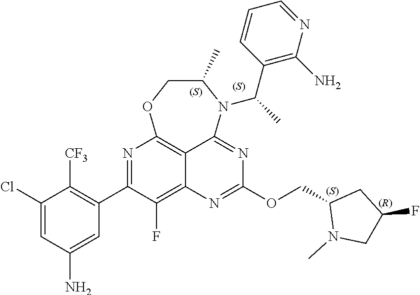 286 & 287 |
| 338 and 339 | 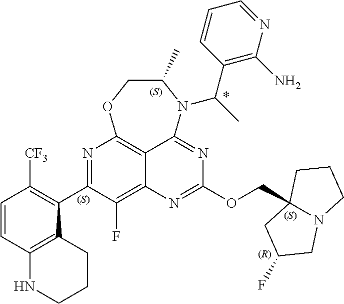 + 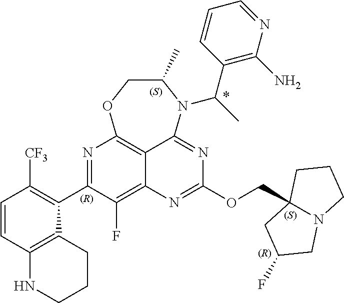 338 & 339 |
| 289 | 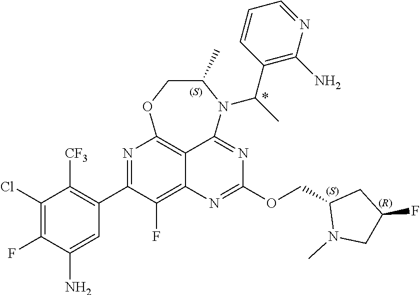 |
| 290 | 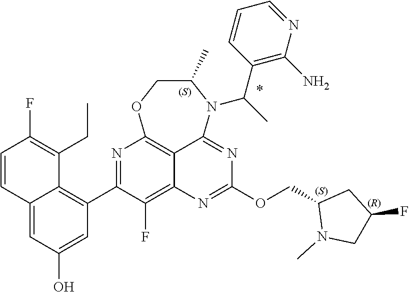 |
| 291 | 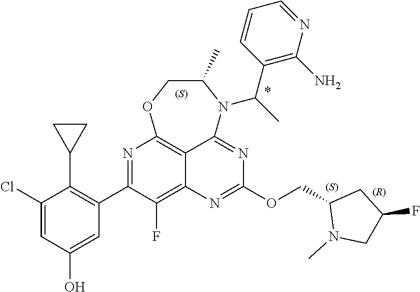 |
| 300 | 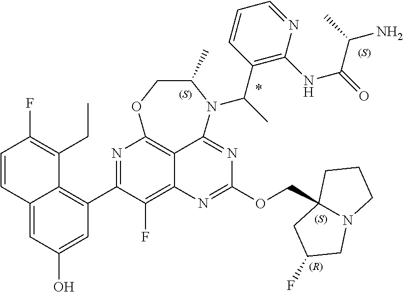 |
| 293 | 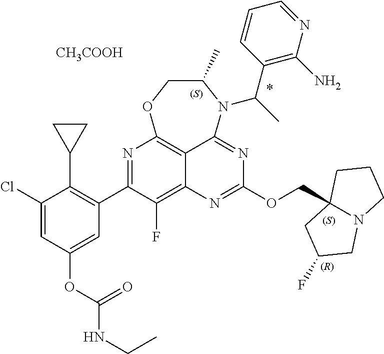 |
| 294 | 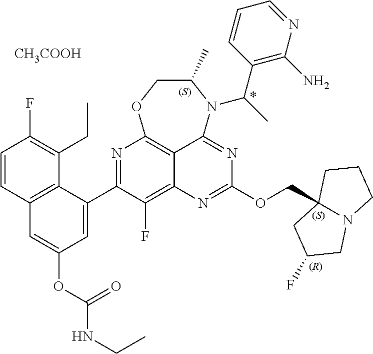 |
| 295 | 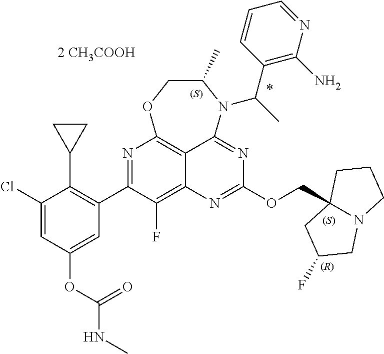 |
| 296 | 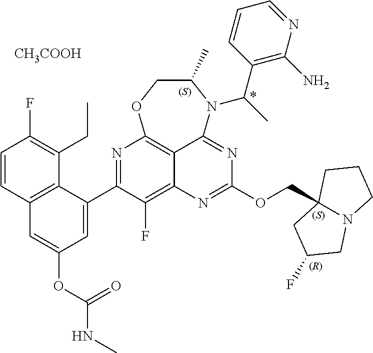 |
| 297 | 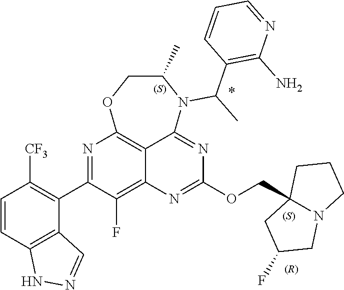 |
| 298 | 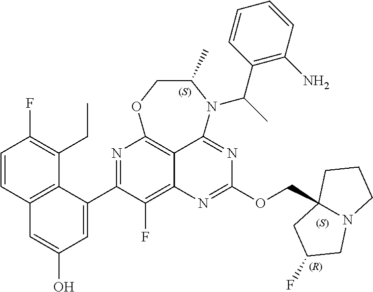 |
| 301 | 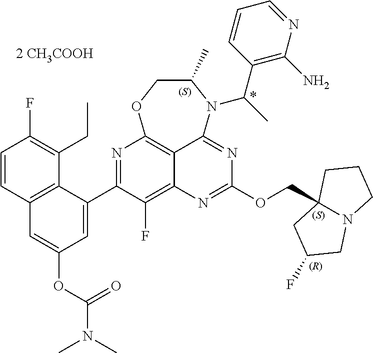 |
| 302 | 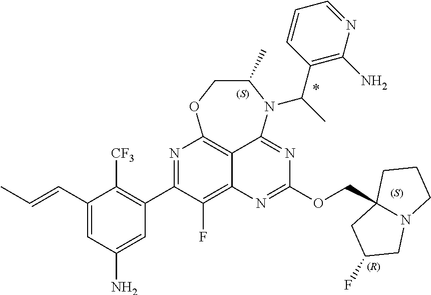 |
| 307 | 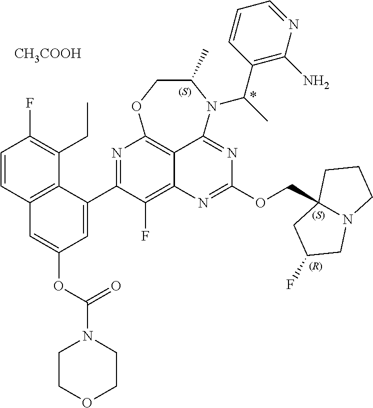 |
| 304 | 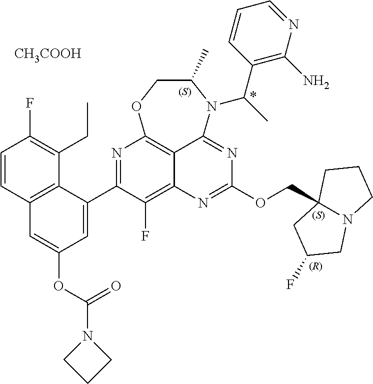 |
| 305 | 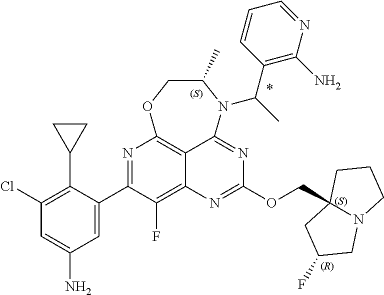 |
| 306 | 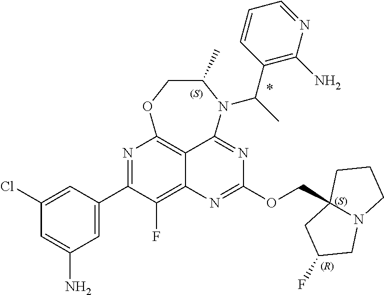 |
| 346 | 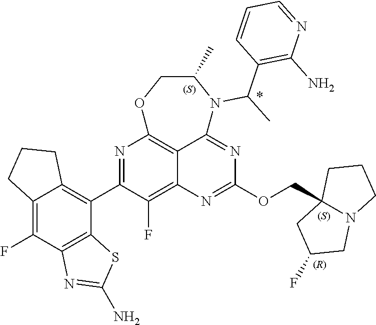 |
| 310 | 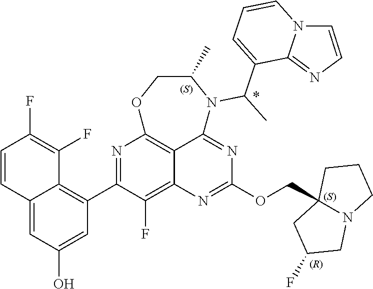 |
| 311 | 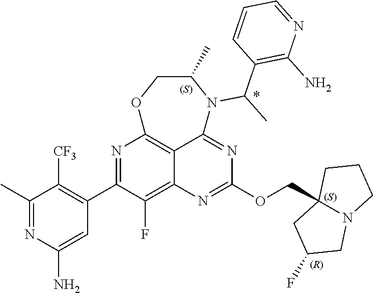 |
| 312 | 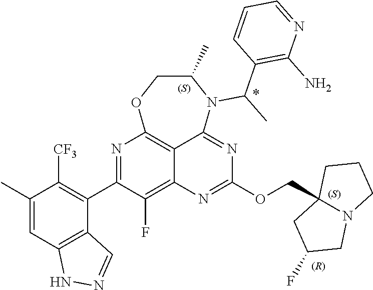 |
| 313 | 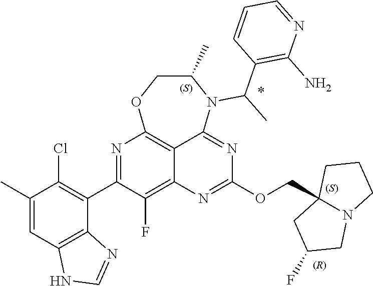 |
| 314 | 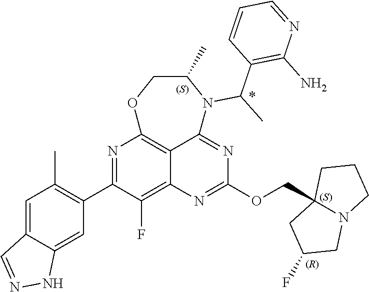 |
| 315 | 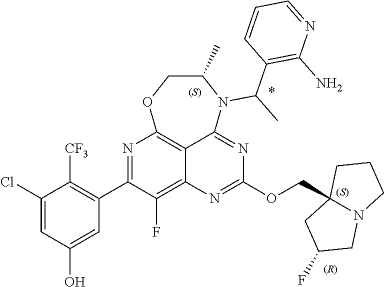 |
| 316 | 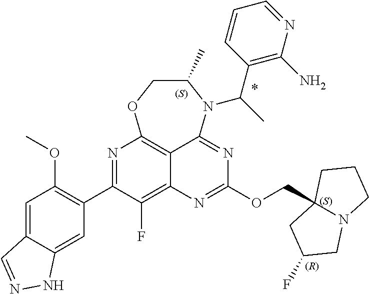 |
| 317 | 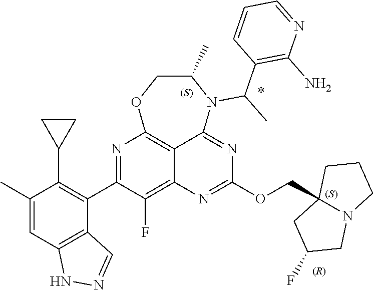 |
| 318 | 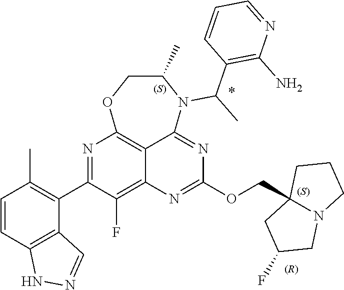 |
| 319 | 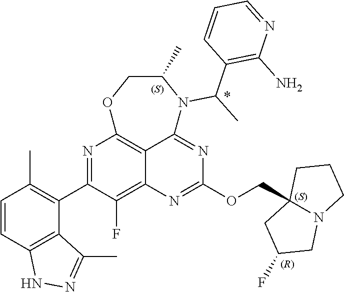 |
| 320 | 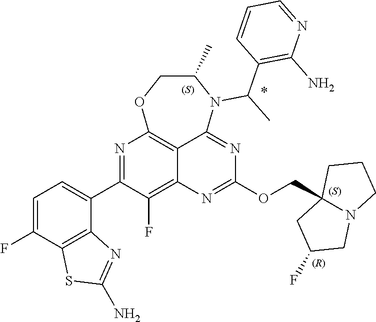 |
| 321 | 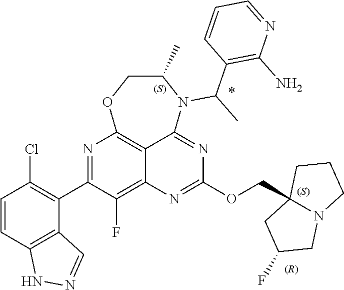 |
| 322 | 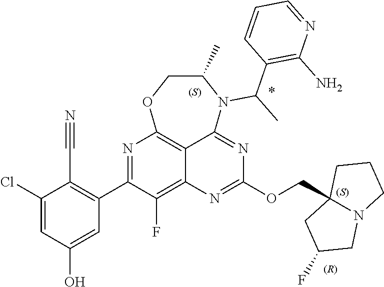 |
| 323 | 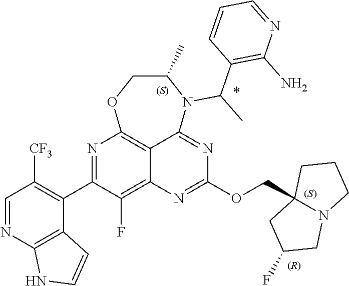 |
| 324 | 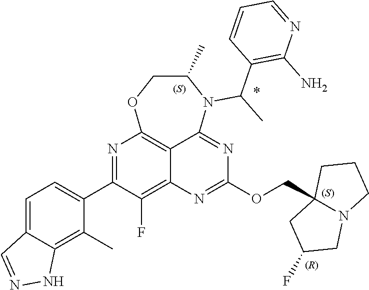 |
| 325 | 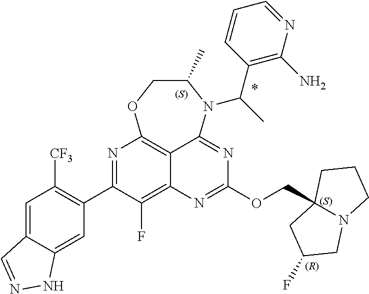 |
| 326 | 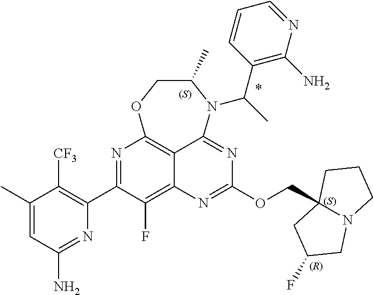 |
| 327 | 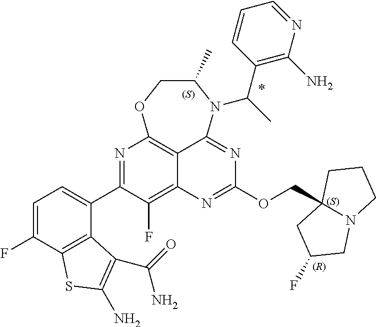 |
| 328 | 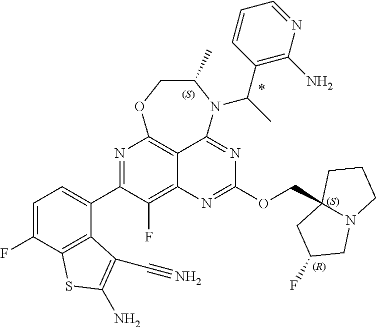 |
| 329 | 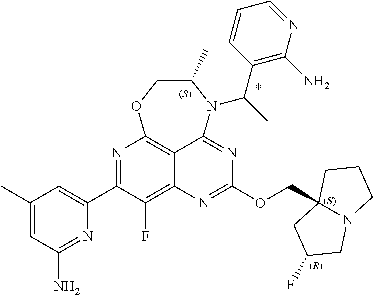 |
| 330 | 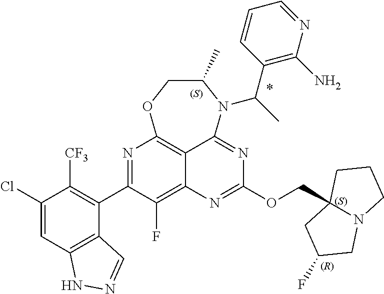 |
| 331 | 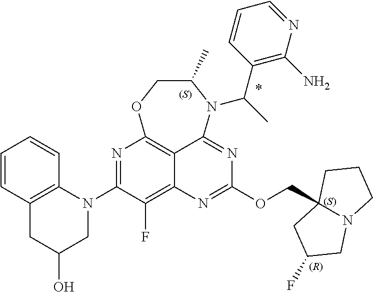 |
| 332 | 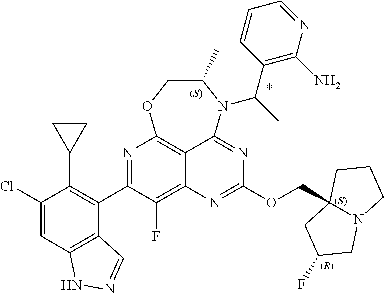 |
| 333 | 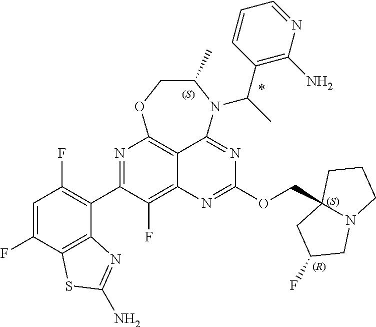 |
| 334 | 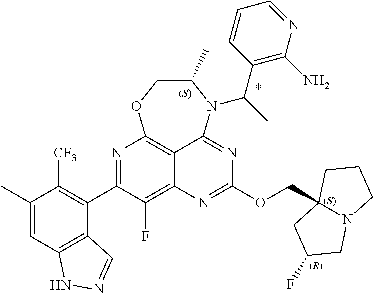 |
| 335 | 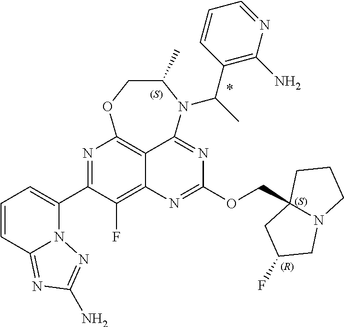 |
| 336 | 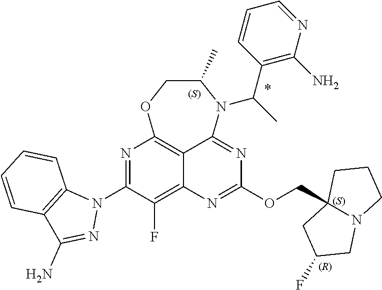 |
| 337 | 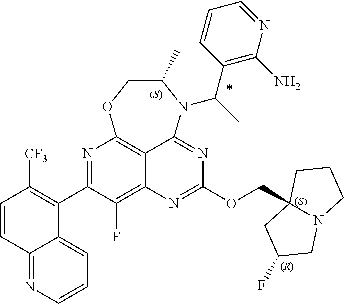 |
| 340 | 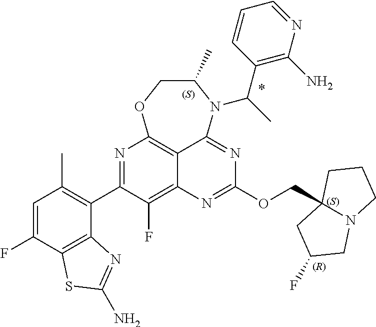 |
| 343 | 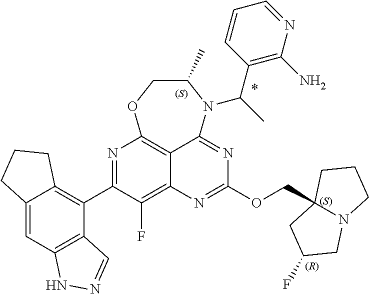 |
| 344 | 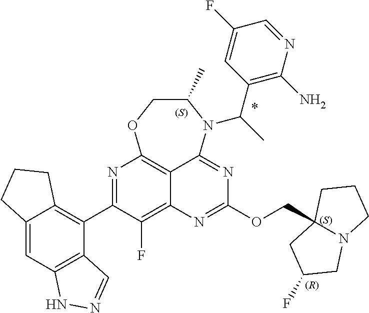 |
| 347 | 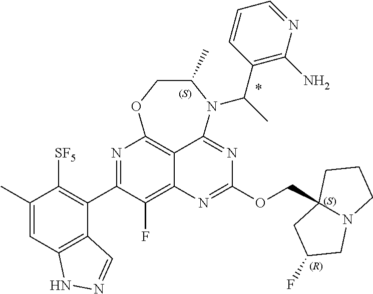 |
| 348 | 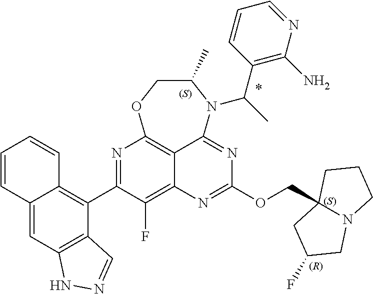 |
| 349 | 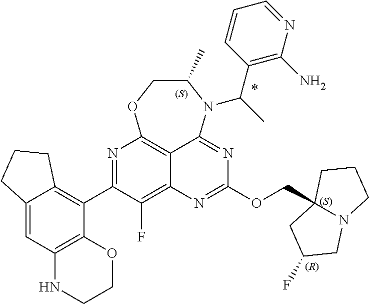 |
| 350 | 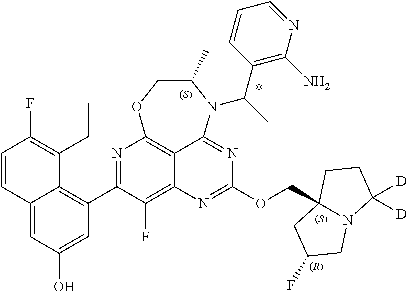 |
| 351 | 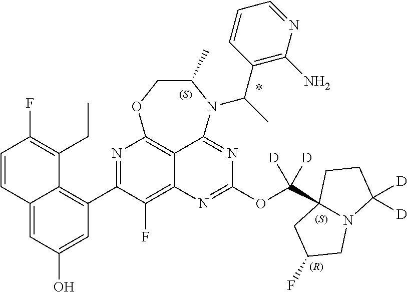 |
| 352 | 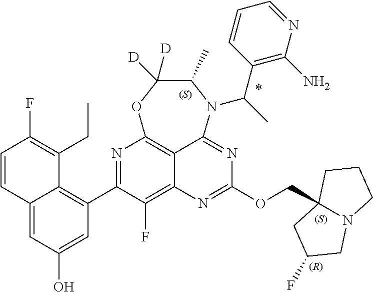 |
| 353 | 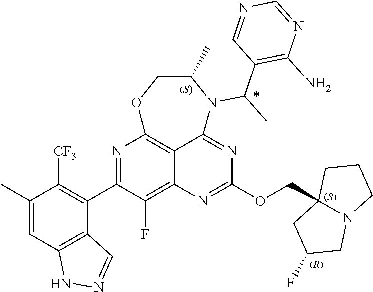 |
| 387 | 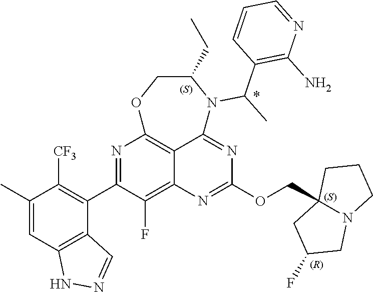 |
| 355 | 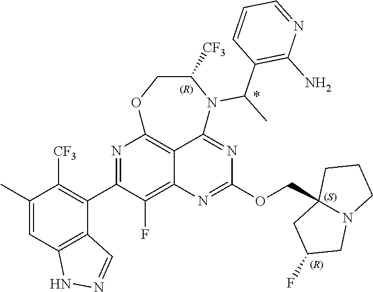 |
| 356 | 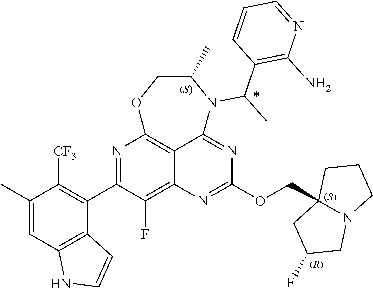 |
| 357 | 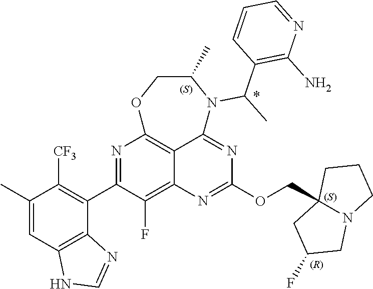 |
| 358 | 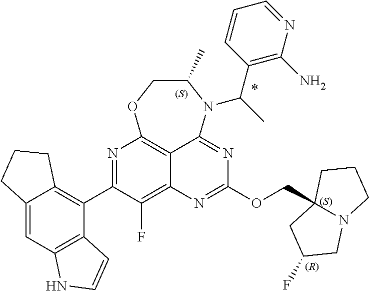 |
| 359 | 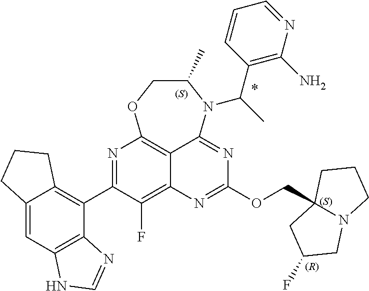 |
| 360 | 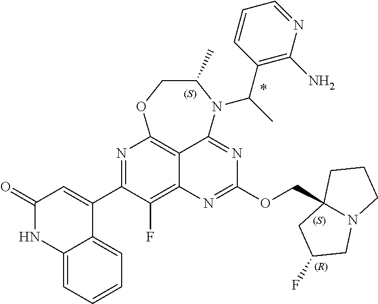 |
| 342 and 364 | 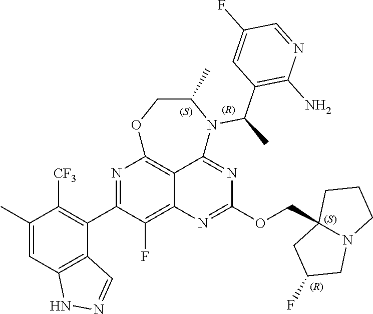 + 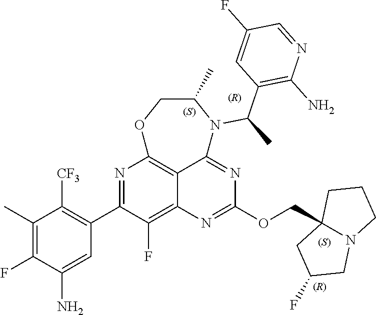 342 & 364 |
| 365 and 366 | 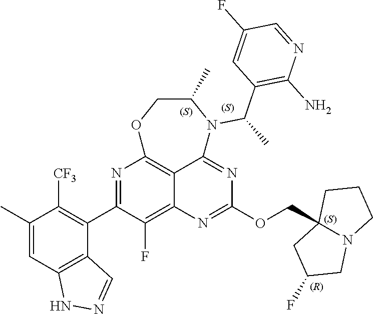 + 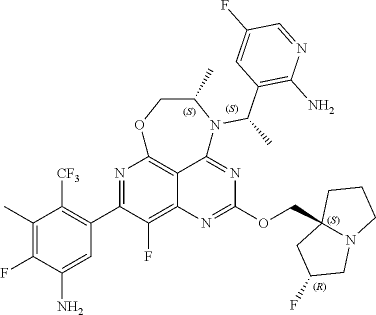 365 & 366 |
| 361 | 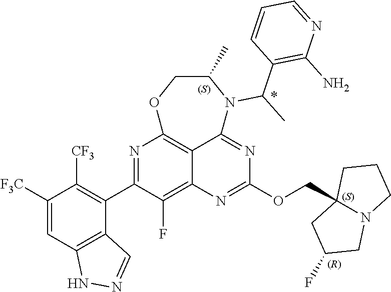 |
| 362 | 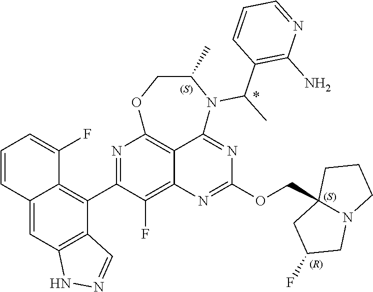 |
| 363 | 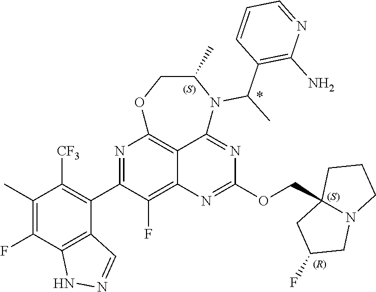 |
| 367 | 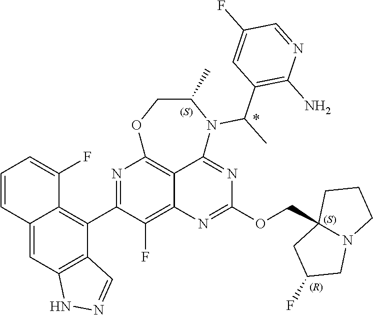 |
| 368 | 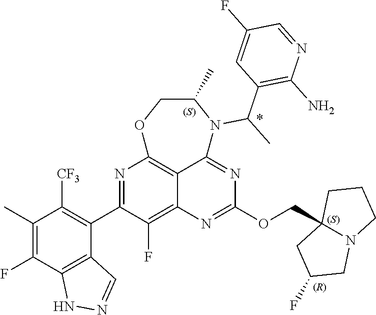 |
| 369 | 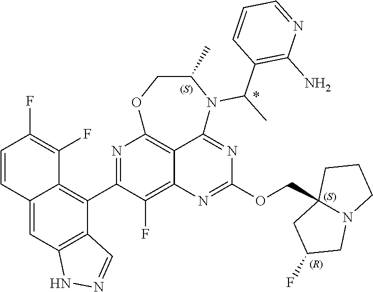 |
| 370 | 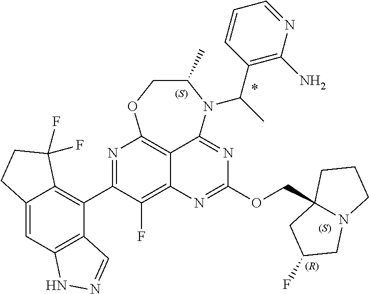 |
| 371 | 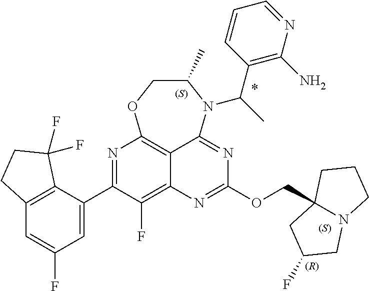 |
| 372 | 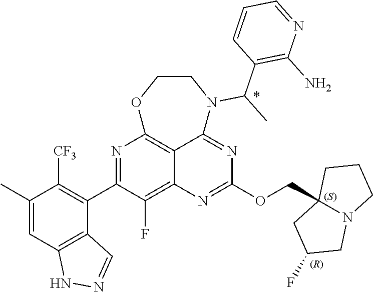 |
| 373 | 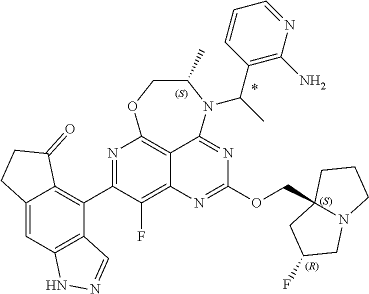 |
| 374 | 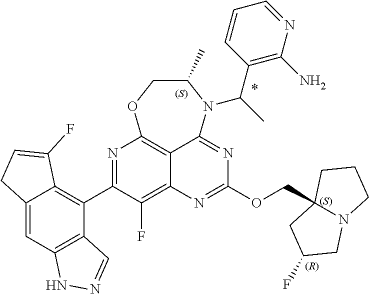 |
| 375 | 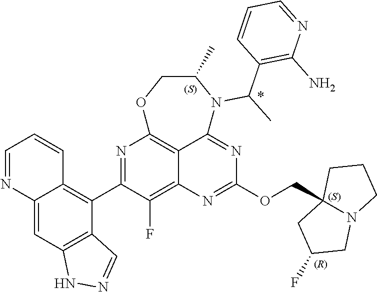 |
| 376 | 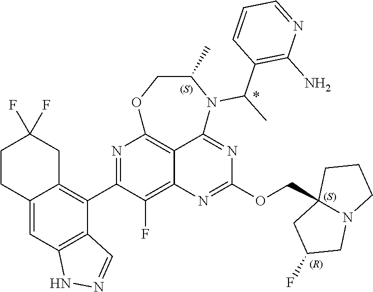 |
| 377 and 378 | 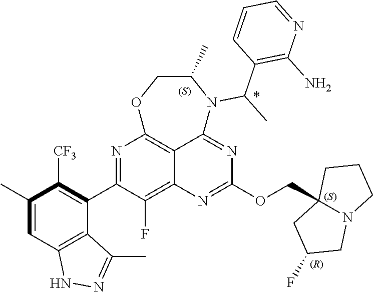 + 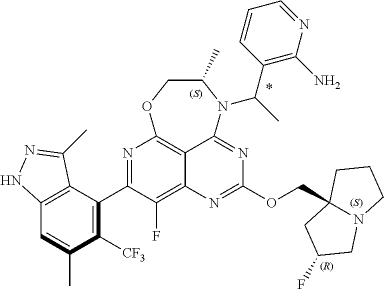 377 & 378 |
| 379 and 380 | 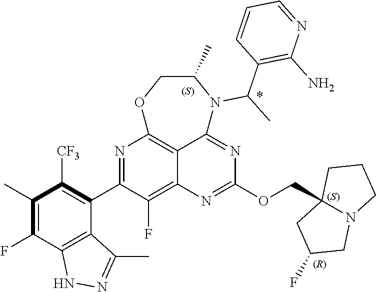 + 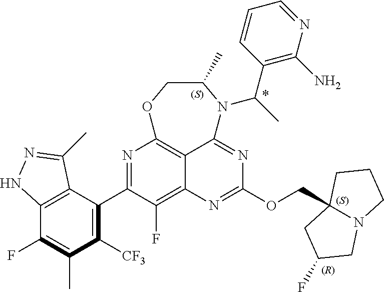 379 & 380 |
| 381 and 382 | 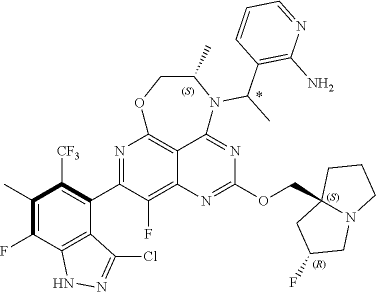 + 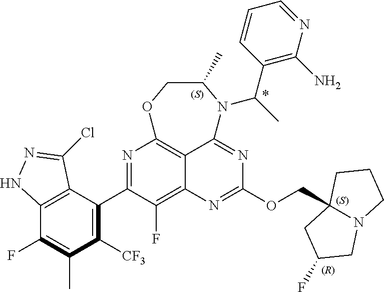 381 & 382 |
| 383 | 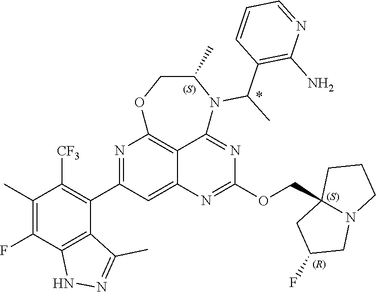 |
| 384 | 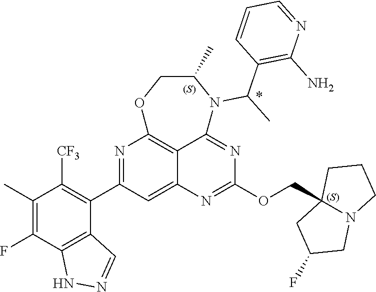 |
| 385 | 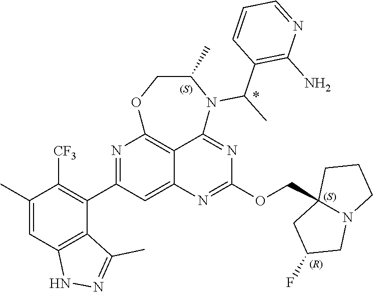 |
| 386 | 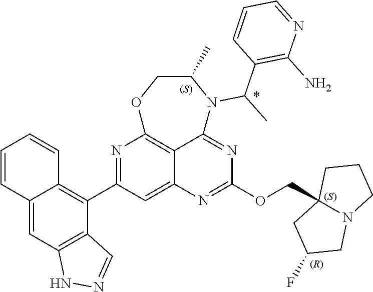 |
| 388 | 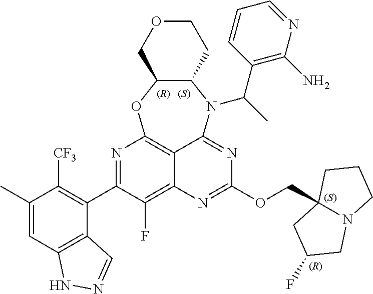 |
| 389 | 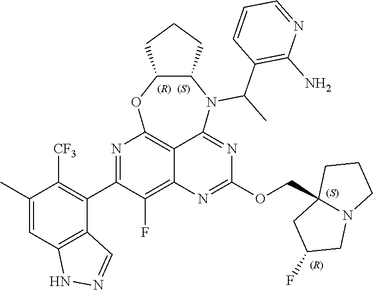 |
| 390 | 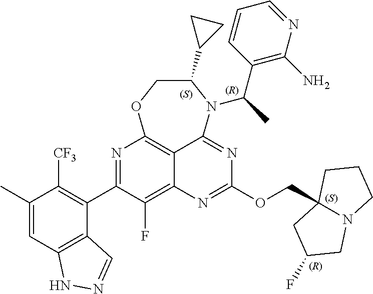 |
| 391 | 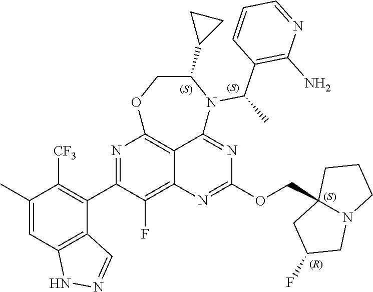 |
| 392 | 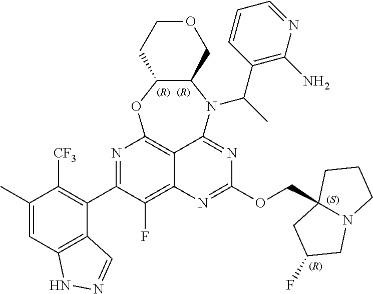 |
| 393 | 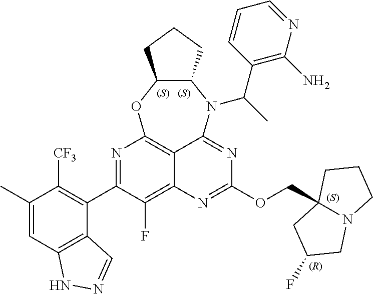 |
| 394 | 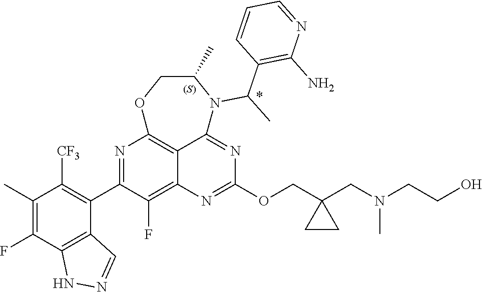 |
| 395 | 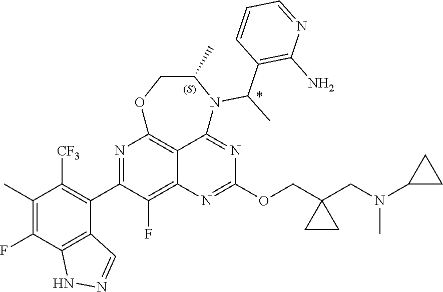 |
| 396 | 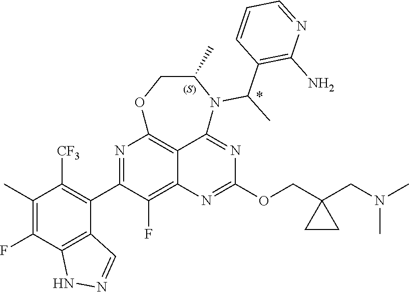 |
| 397 | 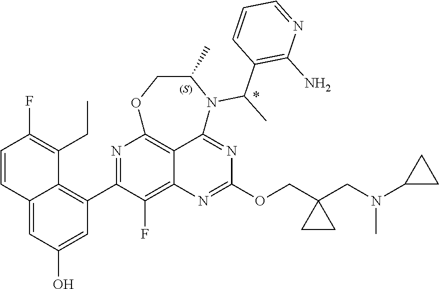 |
| 398 | 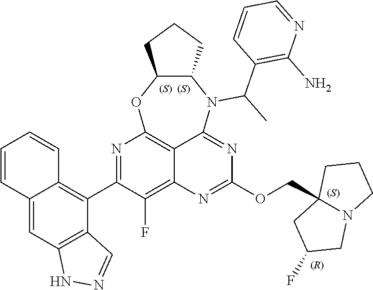 |
| 401 | 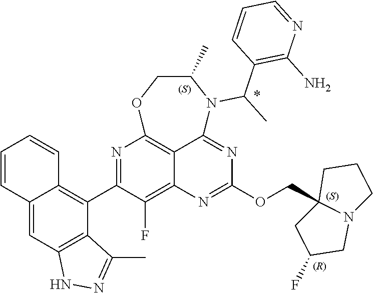 |
| 399 and 400 | 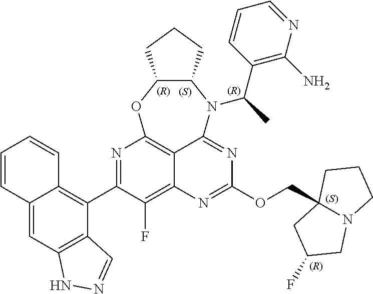 + 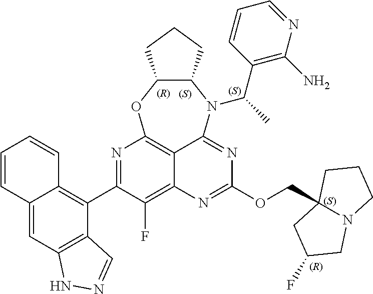 399 & 400 |
| 402 | 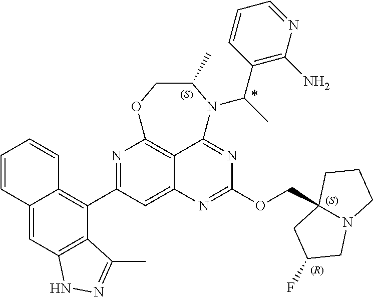 |
| 403 | 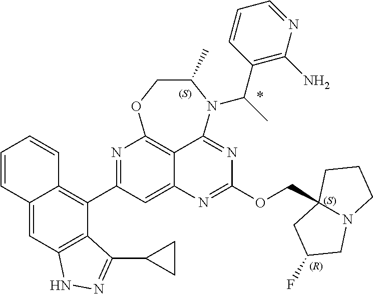 |
| 404 | 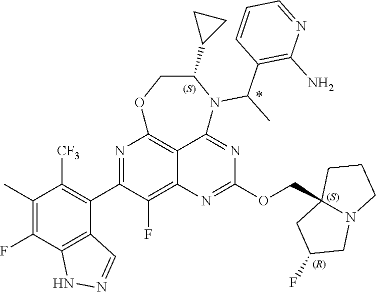 |
| 405 | 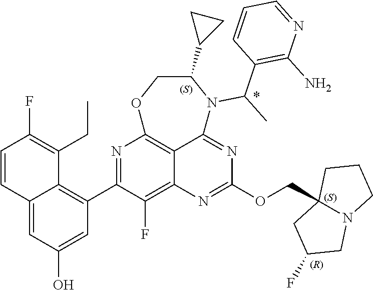 |
| 406 | 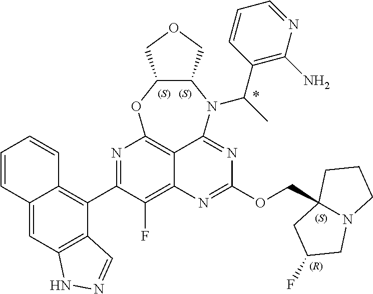 |
| 407 | 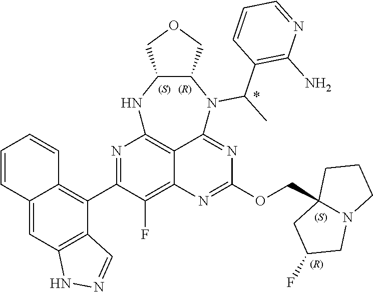 |
| 408 | 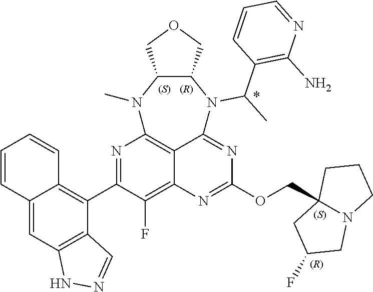 |
| 409 | 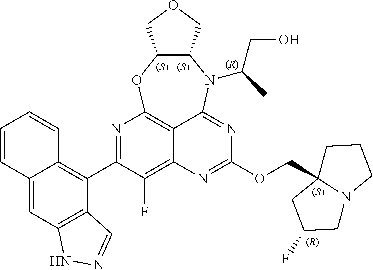 |
| 410 | 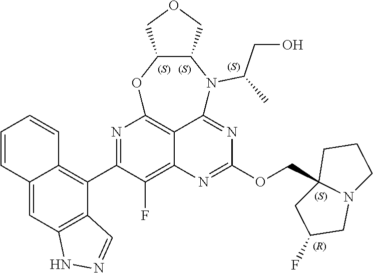 |
| 411 | 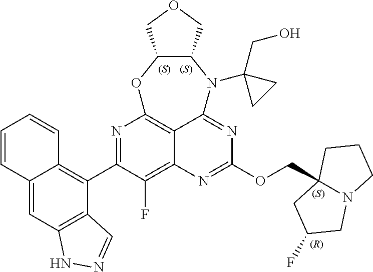 |
| 412 | 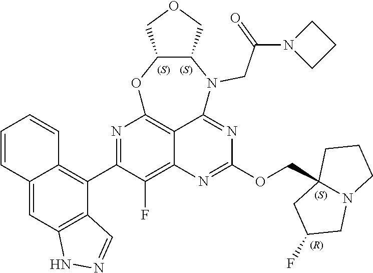 |
| 413 | 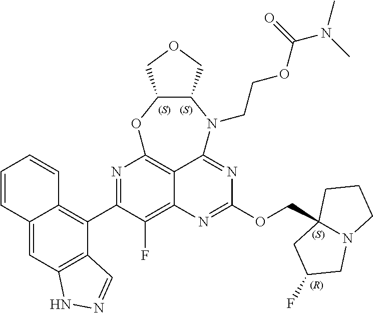 |
| 414 | 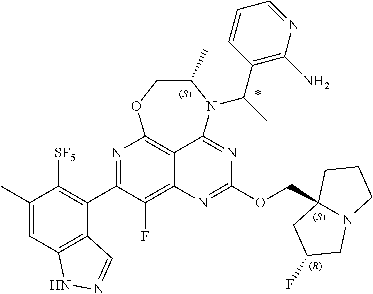 |
| 415 | 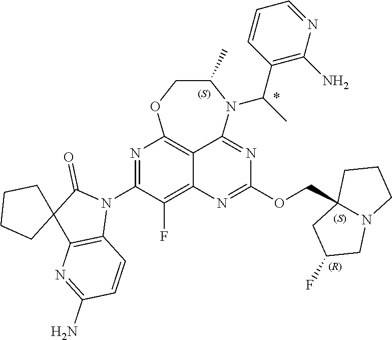 |
| 416 | 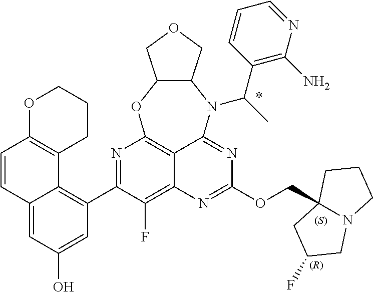 |
| 417 | 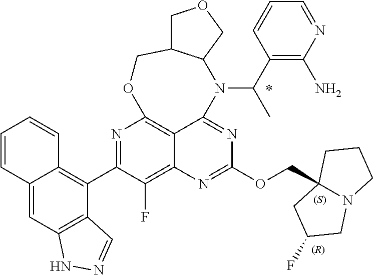 |
| 418 | 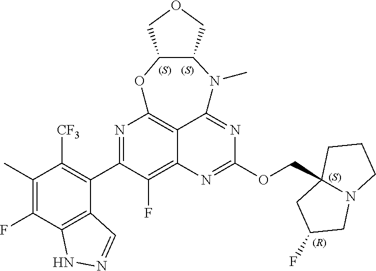 |
| 419 | 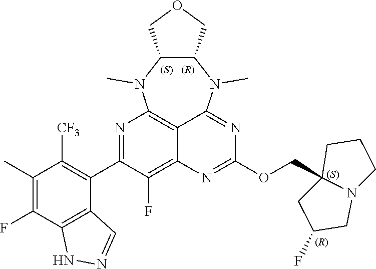 |
| 420 | 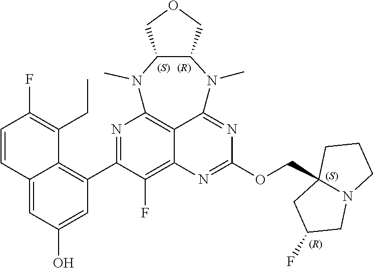 |
| 421 | 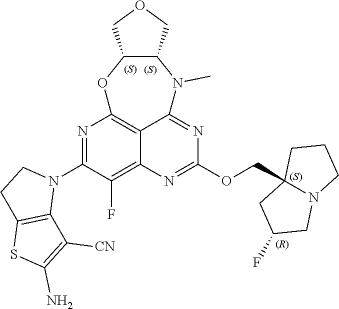 |
| 422 | 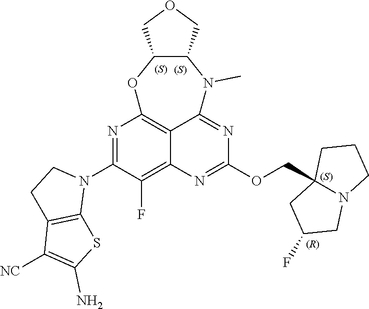 |
| 423 | 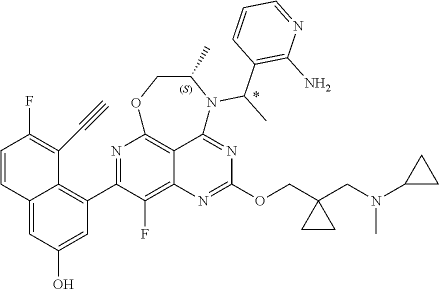 |
| 424 | 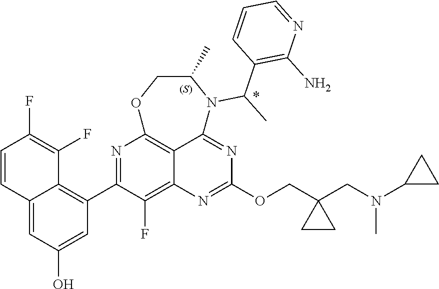 |
| 425 | 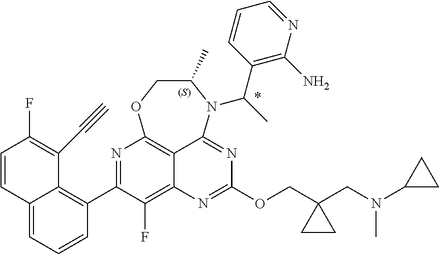 |
| 426 | 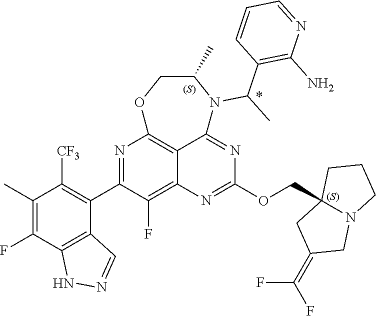 |
| 427 | 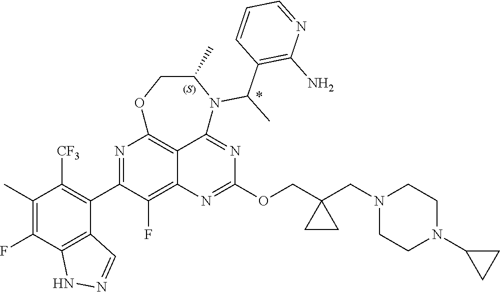 |
| 428 | 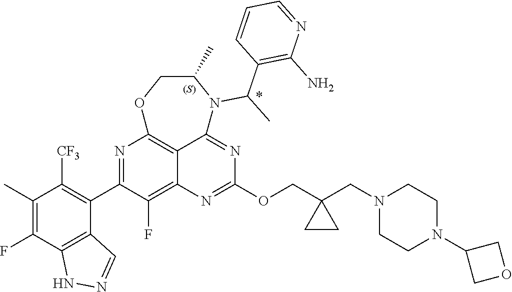 |
| 429 | 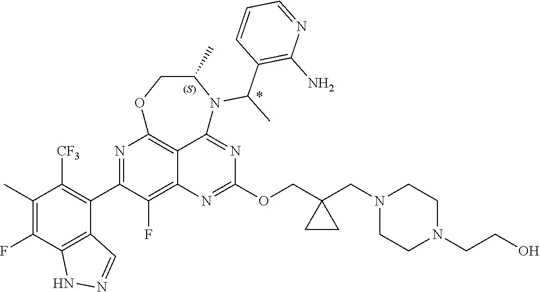 |
| 430 | 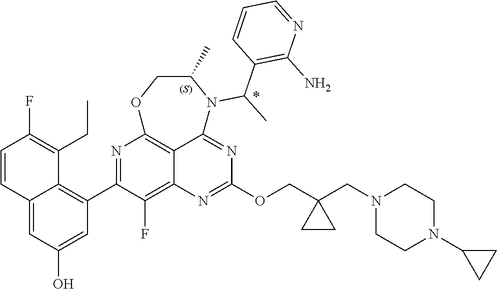 |

28. A pharmaceutical composition, comprising the compound and/or the pharmaceutically acceptable salt thereof according to any one of claims 1-27, and optionally comprising a pharmaceutically acceptable excipient.

29. A method of in vivo or in vitro inhibiting the activity of mutant KRAS proteins, comprising contacting the mutant KRAS proteins with an effective amount of the compound and/or the pharmaceutically acceptable salt thereof according to any one of claims 1-27.

30. A method of treating or preventing a disease in a subject, comprising administering to the subject in need thereof an effective amount of the compound and/or the pharmaceutically acceptable salt thereof according to any one of claims 1-27, wherein the disease is characterized by containing KRAS mutations or KRAS gene amplification; the KRAS mutations are preferably KRAS G12C mutation, KRAS G12D mutation, KRAS G12V mutation, KRAS G13D mutation, and/or KRAS Q61H mutation; the KRAS gene amplification is preferably wild-type KRAS gene amplification; the disease is preferably cancer; and the cancer is preferably a solid tumor or hematologic malignancy; the cancer is more preferably selected from lung cancer (such as lung adenocarcinoma, non-small cell lung cancer, and small cell lung cancer), colorectal cancer, pancreatic cancer, colon cancer, rectal cancer, thyroid carcinoma, esophageal cancer (such as esophageal adenocarcinoma), cholangiocarcinoma, gallbladder carcinoma, head and neck cancer, breast cancer, endometrial cancer, prostate cancer, ovarian cancer, cervical cancer, neuroblastoma, melanoma, brain tumor, gastric cancer (such as chromosomal instability gastric cancer, and gastric adenocarcinoma), bladder cancer, liver cancer, renal cancer, bone cancer, sarcoma, adrenal carcinoma, leukemia, lymphoma and myeloma; and the cancer is further preferably selected from lung cancer (such as lung adenocarcinoma, non-small cell lung cancer, and small cell lung cancer), colorectal cancer, pancreatic cancer, colon cancer, esophageal cancer (such as esophageal adenocarcinoma), and gastric cancer (such as chromosomal instability gastric cancer and gastric adenocarcinoma).

31. Use of the compound and/or the pharmaceutically acceptable salt thereof according to any one of claims 1-27 in the manufacture of a medicament for treating or preventing a disease containing KRAS mutations or KRAS gene amplification, wherein the KRAS mutations are preferably KRAS G12C mutation, KRAS G12D mutation, KRAS G12V mutation, KRAS G13D mutation, and/or KRAS Q61H mutation; the KRAS gene amplification is preferably wild-type KRAS gene amplification; the disease is preferably cancer; and the cancer is preferably a solid tumor or hematologic malignancy; the cancer is more preferably selected from lung cancer (such as lung adenocarcinoma, non-small cell lung cancer, and small cell lung cancer), colorectal cancer, pancreatic cancer, colon cancer, rectal cancer, thyroid carcinoma, esophageal cancer (such as esophageal adenocarcinoma), cholangiocarcinoma, gallbladder carcinoma, head and neck cancer, breast cancer, endometrial cancer, prostate cancer, ovarian cancer, cervical cancer, neuroblastoma, melanoma, brain tumor, gastric cancer (such as chromosomal instability gastric cancer, and gastric adenocarcinoma), bladder cancer, liver cancer, renal cancer, bone cancer, sarcoma, adrenal carcinoma, leukemia, lymphoma and myeloma; and the cancer is further preferably selected from lung cancer (such as lung adenocarcinoma, non-small cell lung cancer, and small cell lung cancer), colorectal cancer, pancreatic cancer, colon cancer, esophageal cancer (such as esophageal adenocarcinoma), and gastric cancer (such as chromosomal instability gastric cancer and gastric adenocarcinoma).

32. The compound and/or the pharmaceutically acceptable salt thereof according to any one of claims 1-27, for use as a medicament.

33. The compound and/or the pharmaceutically acceptable salt thereof according to any one of claims 1-27, for use in treating or preventing a disease containing KRAS mutations or KRAS gene amplification, wherein the disease is preferably cancer; the KRAS mutations are preferably KRAS G12C mutation, KRAS G12D mutation, KRAS G12V mutation, KRAS G13D mutation, and/or KRAS Q61H mutation; the KRAS gene amplification is preferably wild-type KRAS gene amplification; and the cancer is preferably a solid tumor or hematologic malignancy; the cancer is more preferably selected from lung cancer (such as lung adenocarcinoma, non-small cell lung cancer, and small cell lung cancer), colorectal cancer, pancreatic cancer, colon cancer, rectal cancer, thyroid carcinoma, esophageal cancer (such as esophageal adenocarcinoma), cholangiocarcinoma, gallbladder carcinoma, head and neck cancer, breast cancer, endometrial cancer, prostate cancer, ovarian cancer, cervical cancer, neuroblastoma, melanoma, brain tumor, gastric cancer (such as chromosomal instability gastric cancer, and gastric adenocarcinoma), bladder cancer, liver cancer, renal cancer, bone cancer, sarcoma, adrenal carcinoma, leukemia, lymphoma and myeloma; and the cancer is further preferably selected from lung cancer (such as lung adenocarcinoma, non-small cell lung cancer, and small cell lung cancer), colorectal cancer, pancreatic cancer, colon cancer, esophageal cancer (such as esophageal adenocarcinoma), and gastric cancer (such as chromosomal instability gastric cancer and gastric adenocarcinoma).

34. A pharmaceutical combination, comprising the compound and/or the pharmaceutically acceptable salt thereof according to any one of claims 1-27, and at least one additional therapeutic agent, wherein the additional therapeutic agent is preferably selected from an anti-neoplastic active agent, an anti-inflammatory agent or an immunomodulator, wherein the anti-neoplastic active agent includes a chemotherapeutic agent, an immune checkpoint inhibitor or agonist, and a targeted therapeutic agent.

35. A compound of formula (II): 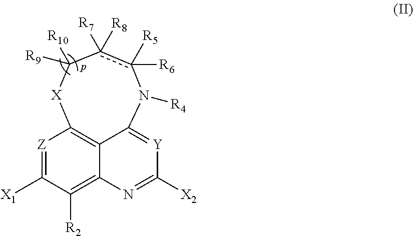
or a pharmaceutically acceptable salt thereof, or a solvate, a racemic mixture, an enantiomer, a diastereomer or a tautomer thereof, wherein R₂, R₄, R₅, R₆, R₇, R₈, R₉, R₁₀, X, Y, Z and p are as defined in any one of claims 1-26;
X₁ is halogen; and
X₂ is hydrogen, halogen, —S(C₁₋₆ alkyl) or —S(O)(C₁₋₆ alkyl).

36. The compound or the pharmaceutically acceptable salt thereof, or the solvate, the racemic mixture, the enantiomer, the diastereomer or the tautomer thereof according to claim 35, wherein the compound is a compound of formula (II-1): 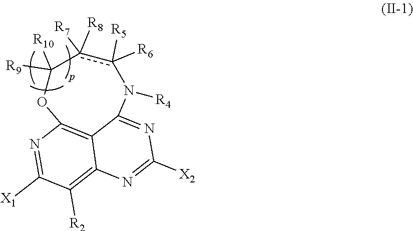
wherein X₁ is halogen; and preferably, X₁ is chlorine; and
X₂ is hydrogen, halogen, —S(C₁₋₆ alkyl) or —S(O)(C₁₋₆ alkyl); preferably, X₂ is hydrogen, chlorine, —S(CH₃) or —S(O)(CH₃); and more preferably, X₂ is hydrogen, —S(CH₃) or —S(O)(CH₃).

37. The compound or the pharmaceutically acceptable salt thereof, or the solvate, the racemic mixture, the enantiomer, the diastereomer or the tautomer thereof according to claim 36, which is selected from: 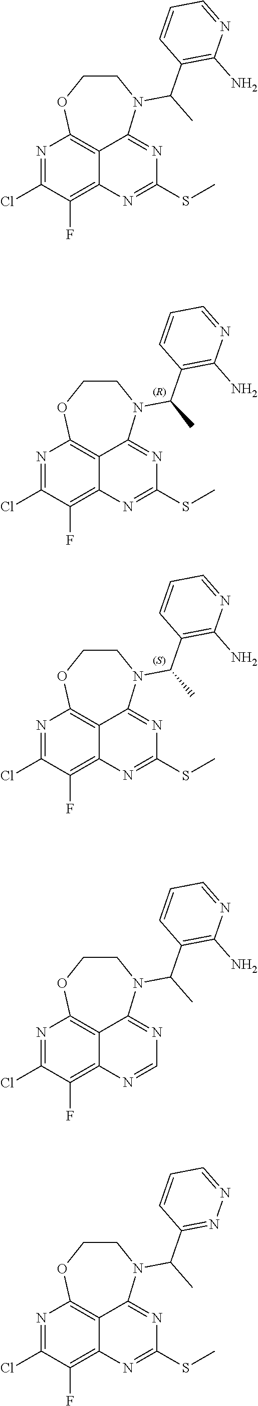 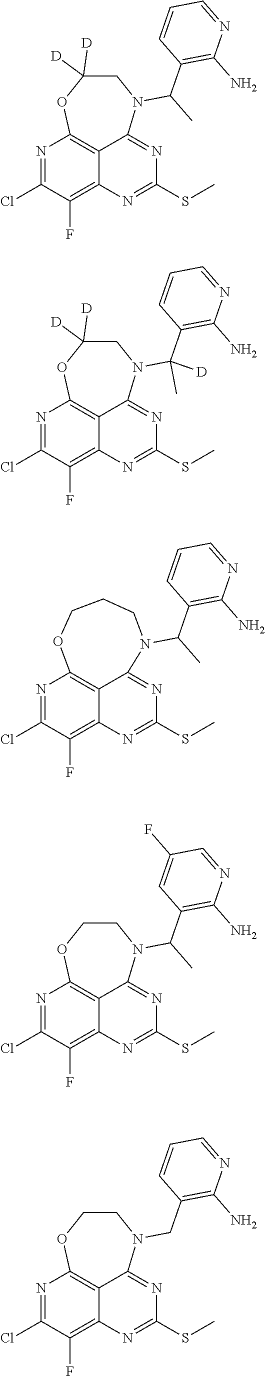 [SMILES: [2H]C1([2H])CN(C(C)c2cccnc2N)c2nc(SC)nc3c(F)c(Cl)nc(c23)O1 ; [2H]C1([2H])CN(C([2H])(C)c2cccnc2N)c2nc(SC)nc3c(F)c(Cl)nc(c23)O1 ; CSc1nc2c3c(nc(Cl)c(F)c3n1)OCCCN2C(C)c1cccnc1N ; CSc1nc2c3c(nc(Cl)c(F)c3n1)OCCN2C(C)c1cc(F)cnc1N ; CSc1nc2c3c(nc(Cl)c(F)c3n1)OCCN2Cc1cccnc1N] 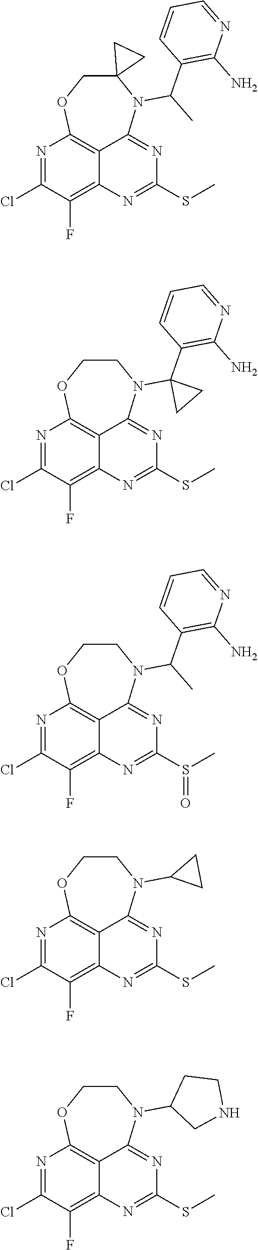 [SMILES: CSc1nc2c3c(nc(Cl)c(F)c3n1)OCC1(CC1)N2C(C)c1cccnc1N ; CSc1nc2c3c(nc(Cl)c(F)c3n1)OCCN2C1(c2cccnc2N)CC1 ; CC(c1cccnc1N)N1CCOc2nc(Cl)c(F)c3nc(S(C)=O)nc1c23 ; CSc1nc2c3c(nc(Cl)c(F)c3n1)OCCN2C1CC1 ; CSc1nc2c3c(nc(Cl)c(F)c3n1)OCCN2C1CCNC1] 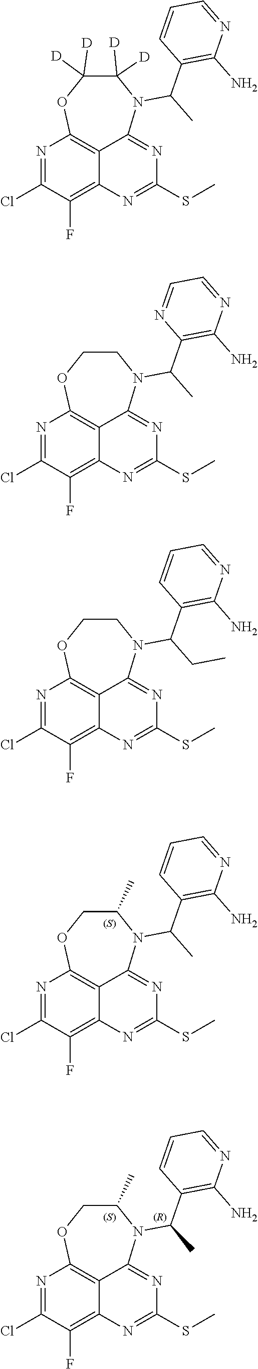 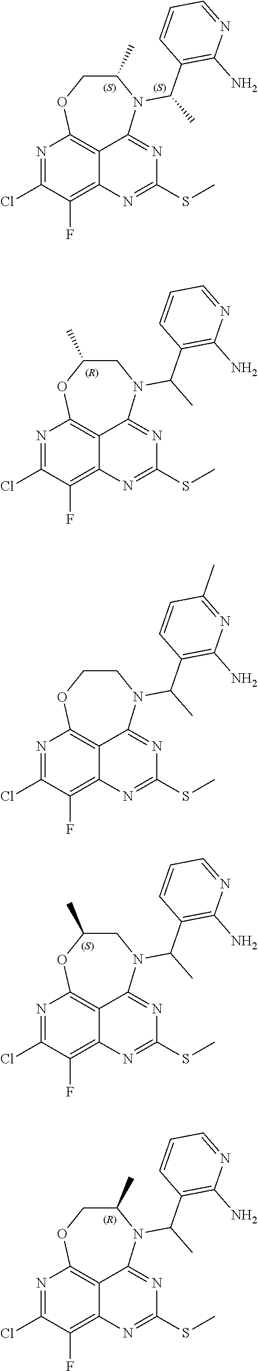 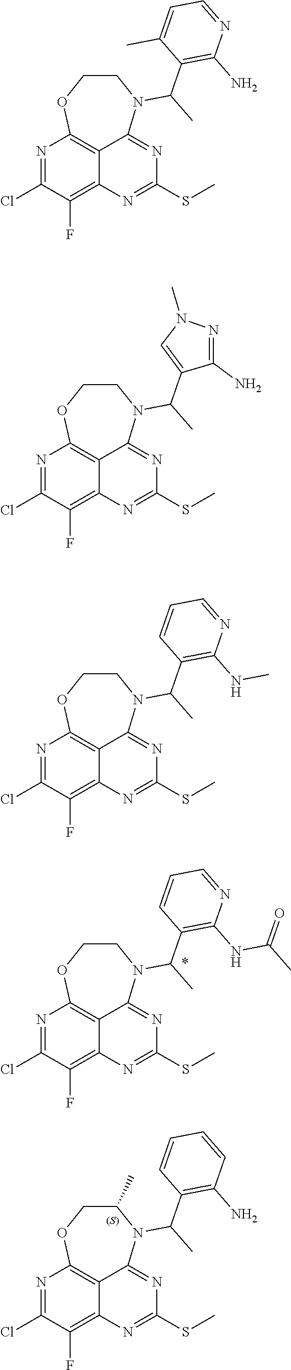 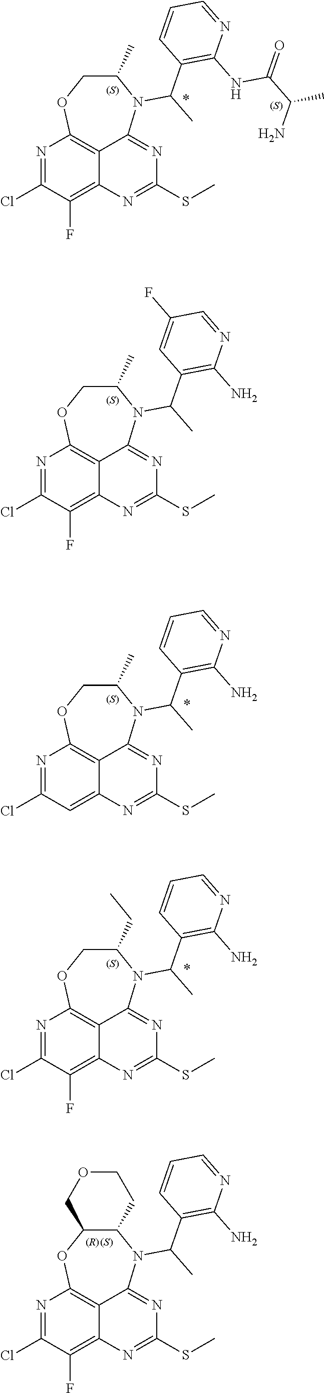 [chemical structure]
